(19) United States
(12) Patent Application Publication
(10) Pub. No.: US 2012/0192315 A1
(43) Pub. Date: Jul. 26, 2012

(54) RESISTANCE GENES AND PROTEINS ACTIVE AGAINST FUSARIUM ROOT ROTS, CYST NEMATODES AND SOYBEAN SUDDEN DEATH SYNDROME AND METHODS EMPLOYING SAME

(75) Inventor: David A. Lightfoot, Carbondale, IL (US)

(73) Assignee: Southern Illinois University Carbondale, Carbondale, IL (US)

(21) Appl. No.: 13/350,672

(22) Filed: Jan. 13, 2012

Related U.S. Application Data
(60) Provisional application No. 61/432,226, filed on Jan. 13, 2011.

(51) Int. Cl. A01H 5/00; G01N 33/566; C07K 16/00; A01H 5/10; C12Q 1/68

(52) U.S. Cl. 800/298; 435/6.11; 435/6.19; 530/389.1; 436/501

(57) ABSTRACT

Fusarium root rot, cyst nematode and soybean sudden death syndrome resistance genes; Fusarium root rot, cyst nematode and soybean sudden death syndrome resistance proteins; Fusarium root rot, cyst nematode and soybean sudden death syndrome resistant plant lines, and methods of breeding and engineering same.

### CROSS REFERENCE TO RELATED APPLICATIONS

This application is based on and claims priority to U.S. Provisional Application Ser. No. 61/432,226 filed Jan. 13, 2011, the entire contents of which is herein incorporated by reference.

### TECHNICAL FIELD

The present invention relates to plant breeding and plant genetics. More particularly, the invention relates to soybean cyst nematode and soybean sudden death syndrome resistance genes, soybean cyst nematode and soybean sudden death syndrome resistant soybean lines, and methods of breeding and engineering the same.

### BACKGROUND OF THE INVENTION

Soybeans are a major cash crop and investment commodity in North America and elsewhere. Soybean oil is one of the most widely used edible oils, and soybeans are used worldwide both in animal feed and in human food production.

Fusarium virguliforme agent of Fusarium root rot (FRR) on many plant species and sudden death syndrome (SDS) only on soybean (Glycine max L. Merr.) has caused increasing seed yield losses in the north central US since 1990 [1]. SDS was found, only in the Americas, and not until 1987 and may be a disease adapting to parasitize soybean from its many other crop hosts. SDS continues its spread toward highly productive soybean-cultivated soils. Known as a fungus of the genus Fusarium that attacks the roots of soybeans, it reproduces very quickly, survives in the soil for many years in the absence of a soybean crop, and can cause substantial soybean crop yield losses.

The soybean cyst nematode (SCN), Heterodera glycines, is a widespread pest of soybeans in the American continent. It is an ancient pest of soybean. It was first named in Japan more than 75 years ago. Since the first US reports in North Carolina in 1954, SCN continues its spread toward almost all soybean-cultivated soils. Known as a small plant-parasitic roundworm that attacks the roots of soybeans, it reproduces very quickly, survives in the soil for many years in the absence of a soybean crop, and can cause substantial soybean crop yield losses.

Resistant soybean varieties are an effective tool available for both SDS and SCN management. Partial resistance to SDS in soybean was found often associated with resistance to SCN across germplasm suggesting the two pathogens may be recognized by a common resistance mechanisms. There are multiple sources for soybean cyst nematode resistance genes in commercial soybean varieties (PI88788, Peking and PI209332), and several have been used to develop cultivars (Myers & Anand (1991), Euphytica 55:197-201; Rao-Arrelli et al. (1988) Crop Sci 28:650-652). All the described loci involved in the resistance to SCN were reported to be quantitative. (Concibido et al. (1997) Crop Sci 37:258-264; Concibido (1996) Theor Appl Genet. 93:234-241; Webb et al. (1995) Theor Appl Genet. 91:574-581; Rao-Arrelli et al. (1992) Crop Sci 32:862-864; Matthews et al. (1991) Soybean Genetics Newsletter, Rao-Arrelli et al., 1988). Major genes for resistance differ by their which chromosome they are on (chromosome no. 8, 18, 11, 17, 20 13, 16 and 15) and their chromosomal positions (LG A2, G, B, I, F, J and E) and race of the pathogen against which they confer the resistance (e.g. Race 1, 3, 5 or 14). SCN resistance is simply inherited, but field resistance is oligogenic due to the existence of variation among SCN populations that are described as “races” (Riggs and Schmidt (1988) J Nematol 20:392-395).

One gene with two activities and two names, Rfs2/rhg1, provides the major portion of resistance to SDS and SCN race 3 across many genotypes derived from Peking (Chang et al. (1997) Crop Sci 372:965-971; Mathews et al. (1998) Theor Appl Genet. 97:1047-1052; Mahalingam et al. (1995) Breed Sci 45:435-445); PI437654 (Prabhu et al. (1999) Crop Sci 39:982-987; Webb et al., 1995), PI88788 (Bell-Johnson et al. (1998) Soybean Genet Newslett 25:115-118; Concibido et al., 1997; Cregan et al. (1999a) Crop Sci 39:1464-1490; Cregan et al. (1999b) Theor Appl Genet. 99:811-818; Cregan et al. (1999c) Theor Appl Genet. 99:918-928), PI209332 (Concibido et al., 1996), or PI90763 (Concibido et al., 1997). Cytological studies suggest PI437654 and Peking derived resistances share mechanisms (pronounced necrosis and cell wall appositions) not seen in PI88788 in response to race 3 (Mahalingham et al. (1996) Genome 39:986-998). These differences in mechanism may derive from distinct alleles at rhg1 and/or other defense associated loci.

DNA molecular markers linked to FRR/CN/SDS resistance loci can be used to develop effective plant breeding strategies. In general, molecular markers are abundant, often co-dominant, and suitable for rapid screening at the seedling stage. Genetic linkage maps of soybean based on RFLP, RAPD, AFLP, SNP, and microsatellite markers have been described. See Brown et al. (1987) Principles and Practice of Nematode Control in Crops, pp 179-232, Academic Press, Orlando Fla.; Concibido et al., 1996; Concibido et al., 1997; Mahalingham et al., 1995; Meksem et al. (1999) Theor Appl Genet. 99:1131-1142; Meksem et al. (2000) Theor Appl Genet. 101: 747-755; Webb et al., 1995; Weiseman et al. (1992) Theor Appl Genet. 85:136-138; Lark et al. (1993) Theor Appl Genet. 86:901-906; Shoemaker and Specht (1995) Crop Sci 35:436-446; Chang et al., 1997; Keim et al. (1997) Crop Sci 37:537-543).

All such markers have a limit of resistance trait predictability based principally on proximity of the marker to the resistance locus. In some cases, the interpretative value of genetic linkage experiments can be augmented through the simultaneous or serial detection of more than one genetic marker, although this also incurs additional time and resources. Thus, there is a need for a reliable cost-effective method for detecting FRR, CN or SDS resistance using genetic markers. Optimally, a genetic marker comprises a resistance gene.

Therefore, it is of particular importance, both to the soybean breeders and to farmers, to identify, genetic loci for resistance to FRR, SCN and SDS. Having knowledge of the loci for resistance to SCN and SDS, those of ordinary skill in the art can breed or engineer SCN and SDS resistant soybeans. Soybean resistance can be further provided to a non-resistant cultivar in combination with other genotypic and phenotypic characteristics required for commercial soybean lines.

### SUMMARY OF THE INVENTION

The present invention discloses an isolated and purified genetic marker associated with FRR/CN/SDS resistance in soybeans, said marker mapping to linkage group G in the soybean genome. Preferably, the marker has a sequence identical to any one of SEQ ID NO: 2. Representative corresponding markers associated with FRR/CN/SDS susceptibility are set forth as SEQ ID NOs: 1, 2 and 4.

The present invention further provides a plant, or parts thereof, which evidences an FRR/CN/SDS resistance response comprising a genome, homozygous with respect to genetic alleles which are native to a first parent and normative to a second parent of the plant, wherein said second parent evidences significantly less resistant response to FRR/CN/SDS than said first parent and said improved plant comprises alleles from said first parent that evidences resistance to FRR/CN/SDS in hybrid combination in at least one locus selected from: a locus mapping to linkage group G and mapped by one or more of the markers set forth as SEQ ID NOs: 1, 2, and 4, said resistance not significantly less than that of the first parent in the same hybrid combination, and yield characteristics which are not significantly different than those of the second parent in the same hybrid combination.

In another embodiment, a plant of the present invention, or parts thereof, comprises the progeny of a cross between first and second inbred lines, alleles conferring FRR/CN/SDS resistance being present in the homozygous state in the genome of one or the other or both of said first and second inbred lines such that the genome of said first and second inbreds together donate to the hybrid a complement of alleles necessary to confer the FRR/CN/SDS resistance. Further disclosed are hybrid plants derived therefrom.

Also disclosed herein are isolated and purified biologically active FRR/CN/SDS resistance polypeptide and an isolated and purified nucleic acid molecule encoding the same are disclosed (SEQ ID NOs: 1, 2, and 3). Preferably, the polypeptide comprises a soybean FRR/CN/SDS resistance polypeptide. Chimeric genes comprising the isolated and purified nucleic acid molecules encoding a FRR/CN/SDS resistance polypeptide are also provided.

In one embodiment, the nucleic acid molecule encoding a FRR/CN/SDS resistance gene comprises an isolated soybean Rfs2/rhg1 gene that confers FRR/CN/SDS resistance to a non-resistant host organism. The gene is capable of conveying Heterodera glycines-infestation resistance, Fusarium spp.-infection resistance, or both Heterodera glycines-infestation resistance or Fusarium spp.-infection resistance to a non-resistant plant germplasm, the gene located within a quantitative trait locus mapping to linkage group G and mapped by genetic markers of SEQ ID NOs: 1, 2, and 4, said gene located along said quantitative trait locus between said markers. Preferably, the polypeptide comprises (a) a polypeptide encoded by a nucleic acid sequence set forth as SEQ ID NO: 3; (b) a polypeptide encoded by a nucleic acid having homology to a DNA sequence set forth as SEQ ID NO: 3; (c) a polypeptide encoded by a nucleic acid capable of hybridizing under stringent conditions to a nucleic acid comprising a sequence or the complement of a sequence set forth as SEQ ID NO: 3; (d) a polypeptide which is a biologically functional equivalent of a peptide set forth as SEQ ID NO: 3; or (e) a polypeptide comprising a fragment of a polypeptide of (a), (b), (c) or (d).

The present invention further provides any small molecule, polypeptide or protein that binds to the protein encoded by the isolated FRR/CN/SDS resistance gene, its promoter region, or functional portion thereof, that provides a mechanism for resistance.

The present invention further provides an isolated FRR/CN/SDS resistance gene promoter region, or functional portion thereof, comprising any part of 82.157 kbp fragment of soybean genomic clones 73P6 (GenBank JN597009.1 GI:357432827; SEQ ID NO:4) between BamHI restriction sites and of 21D9 between HindIII restriction sites. All features recognized therein are claimed as part of the invention. The genomic clones are available from the Forrest BAC library described in Meksem et al (2000) Theor Appl Genet. 101 5/6:747-755, available through Southern Illinois University-Carbondale (Carbondale, Ill.) and Texas A&M University BAC center (College Station, Tex.). Preferably, the isolated promoter region comprises the nucleotide sequence of SEQ ID NO: 1 or a sequence substantially similar to SEQ ID NO: 2. The FRR/CN/SDS resistance gene promoter region can be operably linked to heterologous sequence.

The present invention further provides an isolated FRR/CN/SDS resistance gene promoter region, or functional portion thereof, comprising; pSBHB94 (GenBank gi HQ008939SEQ ID NO 3) a 9.772 kbp fragment from soybean genomic BAC clone 21D9 which overlapped 73p6 for the region encompassing a receptor like kinase (RLK) GmRLK18-1 (gene model Glyma_{—}18_{—}02680 at 1,071 kbp on chromosome 18 of the genome sequence. The genomic clones are available from Southern Illinois University-Carbondale (Carbondale, Ill.) Preferably, the isolated promoter region comprises part the nucleotide sequence of SEQ ID NO: 2 or a sequence substantially similar to SEQ ID NO: 4. The FRR/CN/SDS resistance gene promoter region can be operably linked to heterologous sequence.

Further provided is a method for detecting a nucleic acid molecule that encodes an FRR/CN/SDS resistance polypeptide in a biological sample comprising nucleic acid material is also disclosed. The method comprises: (a) hybridizing an isolated and purified nucleic acid molecule of the present invention under stringent hybridization conditions to the nucleic acid material of the biological sample, thereby forming a hybridization duplex; and (b) detecting the hybridization duplex. Preferably, the isolated and purified nucleic acid molecule comprises any of SEQ ID NOs: 1, 2 and 4.

An assay kit for detecting the presence, in biological samples, of an FRR/CN/SDS resistance polypeptide is also disclosed. In one embodiment, the kit comprises a first container that contains a nucleic acid probe identical or complementary to a segment of at least ten contiguous nucleotide bases of a nucleic acid molecule of the present invention, preferably a nucleotide sequence of any one of SEQ ID NOs: 1, 2 and 4. In another embodiment, the kit comprises an antibody that cross-reacts to any one of the polypeptides encoded by SEQ ID NOs: 1, 2 and 4, or portion thereof.

A method for identifying soybean sudden death syndrome (SDS) resistance or soybean cyst nematode (SCN) resistance in a soybean plant using a SDS resistance gene, a SCN resistance gene, or DNA segments having homology to a SDS resistance gene or to an SCN resistance gene is also disclosed. In one embodiment, the method comprises: (a) probing nucleic acids obtained from the soybean plant with a probe derived from said SDS resistance gene or from said SCN resistance gene or from said DNA segment having homology to said SDS resistance gene or to said SCN resistance gene; and observing hybridization of said probe to said nucleic acids, the presence of said hybridization indicating SDS or SCN resistance in said soybean plant. In another embodiment, the method comprises (a) detecting a molecular marker linked to a quantitative trait locus associated with FRR/CN/SDS resistance, wherein the molecular marker is the sequence set forth as any one of SEQ ID NOs: 1, 2 and 4 and (b) determining the presence of FRR/CN/SDS resistance as detection of the molecular marker and determining the absence of FRR/CN/SDS resistance as failure to detect the molecular marker of (b).

A method of reliably and predictably introgressing FRR/CN/SDS resistance genes into non-resistant soybean germplasm is also disclosed. The method comprises: using one or more nucleic acid markers for marker assisted selection among soybean lines to be used in a soybean breeding program, wherein the nucleic acid markers map to linkage groups G or A2 and wherein the nucleic acid markers are selected from among any of SEQ ID NOs: 1, 2 and 4; and introgressing said resistance gene into said non-resistant soybean germplasm.

A soybean plant, or parts thereof, which evidences a FRR/CN/SDS resistance response is also disclosed. The plant comprises a genome, homozygous with respect to genetic alleles which are native to a first parent and non-native to a second parent of the soybean plant, wherein said second parent evidences significantly less resistant response to FRR/CN/SDS than said first parent, and said improved plant comprises alleles from said first parent that evidences resistance to FRR/CN/SDS in hybrid combination of at least one locus selected from: a locus mapping to linkage group G and mapped by one or more of the markers set forth as SEQ ID NOs: 1, 2, and 4, said resistance not significantly less than that of the first parent in the same hybrid combination, and yield characteristics which are not significantly different than those of the second parent in the same hybrid combination.

The soybean plant, or parts thereof, can further comprise the progeny of a cross between first and second inbred lines, alleles conferring FRR/CN/SDS resistance being present in a homozygous state in the genome of one or the other or both of said first and second inbred lines such that the genome of said first and second inbreds together donate to the hybrid a complement of alleles necessary to confer the FRR/CN/SDS resistance. Thus, an FRR/CN/SDS resistant hybrid, or parts thereof, formed with the soybean plant is also disclosed, as is a soybean plant, or parts thereof, formed by selfing the FRR/CN/SDS resistant hybrid.

A recombinant host cell comprising an isolated and purified nucleic acid molecule of the present invention is also disclosed, as is a transgenic plant having incorporated into its genome an isolated and purified nucleic acid molecule. In one embodiment, the nucleic acid molecule comprises encodes a FRR/CN/SDS resistance polypeptide and is present in said genome in a copy number effective to confer expression in the plant of the FRR/CN/SDS resistance polypeptide. Seeds, parts or progeny of the transgenic plant are also disclosed.

A method for producing an antibody that specifically recognizes a FRR/CN/SDS resistance polypeptide is also disclosed. The method comprises (a) recombinantly or synthetically producing a FRR/CN/SDS resistance polypeptide, or portion thereof; (b) formulating the polypeptide of (a) whereby it is an effective immunogen; (c) administering to an animal the formulation of (b) to generate an immune response in the animal comprising production of antibodies, wherein antibodies are present in the blood serum of the animal; and (d) collecting the blood serum from the animal of (c) comprising antibodies that specifically recognize a FRR/CN/SDS resistance polypeptide. Also provided is an antibody produced by the disclosed method such as SEQ ID NO: 21.

Methods for identifying a candidate compound as a modulator of FRR/CN/SDS resistance activity is also disclosed. Such methods include but are not limited to cell-based assays of FRR/CN/SDS resistance gene expression, assays of specific binding to FRR/CN/SDS regulatory elements, and assays of specific binding to FRR/CN/SDS polypeptides. Optionally, the screening methods are adapted to a high-throughput format.

In one embodiment, the method comprises: (a) exposing a cell sample with a candidate compound to be tested, the cell sample containing at least one cell containing a DNA construct comprising a modulatable transcriptional regulatory sequence of an FRR/CN/SDS resistance-encoding nucleic acid and a reporter gene which is capable of producing a detectable signal; (b) evaluating an amount of signal produced in relation to a control sample; and (c) identifying a candidate compound as a modulator of FRR/CN/SDS resistance activity based on the amount of signal produced in relation to a control sample.

The present invention also provides a method for identifying a substance that regulates FRR/CN/SDS resistance gene expression using a chimeric gene that includes an isolated FRR/CN/SDS resistance gene promoter region operably linked to a reporter gene. According to this method, a gene expression system is established that includes the chimeric gene and components required for gene transcription and translation so that reporter gene expression is assayable. To select a substance that regulates FRR/CN/SDS resistance gene expression, the method further provides the steps of using the gene expression system to determine a baseline level of reporter gene expression in the absence of a candidate regulator; providing a plurality of candidate regulators to the gene expression system; and assaying a level of reporter gene expression in the presence of a candidate regulator. A candidate regulator is selected whose presence results in an altered level of reporter gene expression when compared to the baseline level. Preferably, the isolated FRR/CN/SDS resistance gene promoter region used in this method comprises the sequence of SEQ ID NO: 1, 2 or 4 or functional portion thereof.

In another embodiment, the method comprises using an FRR/CN/SDS regulatory sequence to identify a candidate substance that specifically binds to the regulatory sequence. According to the method, a FRR/CN/SDS regulatory gene sequence is exposed to a candidate substance under conditions suitable for binding to a nucleic acid sequence, and a candidate regulator is selected that specifically binds to the FRR/CN/SDS resistance gene promoter region. Preferably, the isolated FRR/CN/SDS resistance gene promoter region used in this method comprises the sequence of SEQ ID NO: 1, 2 or 4, or functional portion thereof.

In another embodiment, a cell-free assay system is used and comprises: (a) exposing a FRR/CN/SDS polypeptide of the present invention to a candidate compound; (b) assaying binding of the candidate compound to the FRR/CN/SDS polypeptide; and (c) identifying a candidate compound as a putative modulator of FRR/CN/SDS resistance activity based on specific binding of the candidate compound to the FRR/CN/SDS polypeptide. Preferably, the FRR/CN/SDS polypeptide comprises some or all of the amino acids of SEQ ID NO: 3.

A method of modulating FRR/CN/SDS resistance in a plant is also disclosed. The method comprises administering to the plant an effective amount of a substance that modulates expression of an FRR/CN/SDS resistance activity-encoding nucleic acid molecule in the plant to thereby modulate FRR/CN/SDS resistance in the plant. Preferably, the substance that modulates expression of an FRR/CN/SDS resistance activity is discovered by a disclosed method of the present invention.

A method for providing a resistance characteristic to a plant is also disclosed. The method comprises introducing to said plant a construct comprising a nucleic acid sequence encoding an FRR/CN/SDS resistance gene product operatively linked to a promoter, wherein production of the FRR/CN/SDS resistance gene product in the plant provides a resistance characteristic to the plant. The construct can further comprises a vector selected from the group consisting of a plasmid vector or a viral vector. The FRR/CN/SDS resistance gene product comprises a protein having an amino acid sequence of SEQ ID NO: 3. The nucleic acid sequence comprises the nucleotide sequence of SEQ ID NO: 1, 2 or 4 or a nucleic acid that is substantially similar to SEQ ID NO: 1, 2 or 4, and which encodes an FRR/CN/SDS resistance polypeptide.

The resistance characteristic is preferably nematode resistance, fungal resistance or combinations thereof. More preferably, the nematode resistance is H. glycines resistance, even more preferably race 3 H. glycines resistance.

In an alternative embodiment the construct further comprises another nucleic acid molecule encoding a polypeptide that provides an additional desired characteristic to the plant. Optionally, the method further comprises monitoring an insertion point for the construct in the plant genome; and providing for insertion of the construct into the plant genome at a location not associated with the resistance characteristic, the desired characteristic, or both the resistance and the desired characteristic. Preferably, the plant is a soybean plant.

The present invention also provides methods for providing a resistance characteristic to a plant is also disclosed, wherein a combination of genetic and non-genetic techniques is employed. The method comprises introducing to said plant a construct comprising a nucleic acid sequence encoding an FRR/CN/SDS resistance gene product operatively linked to a promoter and provision of a substance that modulates SCS/SDS resistance gene activity, wherein production of the FRR/CN/SDS resistance gene product in the plant, in combination with provision of the FRR/CN/SDS resistance gene modulator, provides a resistance characteristic to the plant.

In present invention, the transgenic plant provided comprises an isolated nucleic acid molecule comprising a nucleotide sequence selected from the group consisting of: a) SEQ ID NO:2 or a nucleotide sequence encoding the amino acid sequence of SEQ ID NO: 2; b) a nucleotide sequence which is the reverse complement of (a). c) a nucleotide sequence encoding a receptor like kinase (RLK) related to GmRLK18-1 (SEQ ID NO: 3). Said nucleic acid molecule of the transgenic plant is operably linked to any promoter. Transgenic progeny or seed from said transgenic plant comprises the nucleic acid molecule as well. Said nucleic acid molecule of the transgenic plant is expressed in epidermis, vascular tissue, meristem, cambium, cortex, pith, leaf, sheath, root, flower, developing ovule or seed. The plant of the transgenic plant is selected from the group consisting of: soybean, bean, pea, canola, cabbage, cauliflower, broccoli, sunflower, potato, tobacco, tomato, carrot, sweet potato, sugarbeet, chicory, lettuce, turnip, radish, spinach, rice, wheat, barley, rye, corn, sorghum and sugarcane, asparagus, onion, garlic, eggplant, pepper, celery, squash, pumpkin, cucumber, apple, pear, quince, melon, plum, cherry, peach, nectarine, apricot, strawberry, grape, raspberry, blackberry, pineapple, avocado, papaya, mango and banana. In one example, the plant is soybean. In one example, the plant is a dicot. In one example the transgenic plant is a monocot selected from the group consisting of soybean, bean, pea, canola, cabbage, cauliflower, broccoli, sunflower, potato, tobacco, tomato. In one example, said nucleotide sequence of the transgenic plant comprises: a) the nucleotide sequence of SEQ ID NO: 4; or b) a nucleotide sequence which is the reverse complement of (a). The transgenic progeny or seed from the exemplary transgenic plant comprises said nucleotide sequence.

Also provided is a method of enhancing resistance against pathogen or disease causing agent in a plant, comprising introducing an expression cassette comprising a nucleic acid molecule encoding the amino acid sequence of SEQ ID NO: 2 or an expression cassette comprising any part of SEQ ID NO: 4 into the plant. In said method, the pathogen or disease causing agent is a nematode or fungus, and the pathogen is selected from the group consisting of: Fusarium spp., Phytophthora spp., Pythium spp., and Rhizoctonia spp. A plant produced by the provided method has enhanced pathogen or disease resistance. In one embodiment, the method of increasing expression of disease resistance genes in a plant comprises introducing an expression cassette comprising a nucleic acid molecule encoding the amino acid sequence of SEQ ID NO: 2 or an expression cassette comprising SEQ ID NO: 1 into the plant.

Further provided is an isolated and purified biologically active Fusarium root rot, cyst nematode or sudden death syndrome (FRR/CN/SDS) resistance polypeptide. The isolated and purified biologically active FRR/CN/SDS resistance polypeptide of claim 16, wherein the encoded polypeptide comprises a soybean FRR/CN/SDS resistance polypeptide. Said isolated and purified biologically active FRR/CN/SDS resistance polypeptide, or functional portion thereof, comprises: (a) a polypeptide encoded by the nucleic acid sequence of SEQ ID NO:2; (b) a polypeptide having the amino acid sequence of SEQ NO:3; (c) a polypeptide encoded by a nucleic acid molecule that is substantially identical to SEQ ID NO:3; (d) a polypeptide having the amino acid sequence of an alloprotein of SEQ ID NO: 3. (e) a polypeptide that is a biological equivalent of a peptide having the amino acid sequence of SEQ ID NO:3; or (f) a polypeptide that is immunologically cross-reactive with an antibody that shows specific binding with a polypeptide of SEQ ID NO:3. In one example said isolated and purified biologically active FRR/CN/SDS resistance polypeptide is modified to be in detectably labeled form.

Provided herein also is an isolated and purified nucleic acid molecule encoding a biologically active FRR/CN/SDS resistance polypeptide. Said nucleic acid molecule encodes a polypeptide comprising a soybean FRR/CN/SDS resistance polypeptide. Said nucleic acid molecule further comprises an isolated soybean Rfs2, rhg1 and SDS resistance gene, said gene capable of conveying Heterodera glycines-infestation resistance, Fusarium virguliforme-infection resistance, or both Heterodera glycines-infestation resistance and Fusarium virguliforme-infection resistance to a non-resistant soybean germplasm, said gene located within a quantitative trait locus mapping to linkage group G on chromosome 18 and mapped to the BAC clone B73P6 (SEQ ID NO: 4) by genetic markers within SEQ ID NO:4, said gene located along said quantitative trait locus with said BAC, between said markers. Said nucleic acid molecule may be further defined as comprising: (a) the nucleotide sequence of any of SEQ ID NO:4 or (b) a nucleotide sequence that is substantially identical to any of SEQ ID NO:4 used alone or with SEQ ID NO:2. In one example, said nucleic acid molecule may be further defined as comprising a 20 base pair nucleotide sequence that is identical to a contiguous 20 base pair nucleotide sequence of SEQ ID NO:4. The nucleic acid sequence of said nucleic acid molecule comprises a DNA sequence that hybridizes to a nucleic acid sequence as set forth as SEQ ID NO:4 under wash stringency conditions represented by a wash solution having about 200 mM salt concentration and a wash temperature of at least about 45°, and that encodes an FRR/CN/SDS resistance polypeptide. In another example, the nucleic acid molecule may be further defined as a DNA segment. In yet another example, the nucleic acid molecule may be positioned under the control of a promoter. In one example, said DNA segment and promoter are operationally inserted into a recombinant vector.

A recombinant host cell comprising the above illustrated nucleic acid molecule is provided. Further provided is a transgenic plant having incorporated into its genome a nucleic acid molecule as described above, and the nucleic acid molecule is present in said genome in a copy number effective to confer expression in the plant of an FRR/CN/SDS resistance polypeptide. Plant seeds, parts, or progeny of a such transgenic plant are also provided.

In one example, a transgenic plant comprises a plant cell that is a recombinant host cell comprising the above illustrated nucleic acid molecule. The plant of such a transgenic plant is selected from the group consisting of: soybean, bean, pea, canola, cabbage, cauliflower, broccoli, sunflower, potato, tobacco, tomato, carrot, sweet potato, sugarbeet, chicory, lettuce, turnip, radish, spinach, rice, wheat, barley, rye, corn, sorghum and sugarcane, asparagus, onion, garlic, eggplant, pepper, celery, squash, pumpkin, cucumber, apple, pear, quince, melon, plum, cherry, peach, nectarine, apricot, strawberry, grape, raspberry, blackberry, pineapple, avocado, papaya, mango and banana. In one example, the plant of the transgenic plant is soybean.

Further provided is an assay kit for detecting the presence, in biological samples, of a nucleic acid molecule encoding an FRR/CN/SDS resistance polypeptide, the kit comprising a first container that contains an antibody to any part of SEQ ID NO:3 including SEQ ID NO:21, and antibody to any proteins encoded by SEQ ID NO: 4, or nucleic acid probe identical or complementary to a segment of at least ten contiguous nucleotide bases of the nucleic acid molecule of the odd-numbered SEQ ID NOs:1-4. The kit may further comprise a detectable moiety. The biological sample of the kit may further comprise chromosomes, and wherein the nucleic acid probe hybridizes to a chromosome. A method for determining the presence or absence of FRR/CN/SDS resistance in a soybean plant, or part thereof is provided, and the method comprises: (a) detecting a molecular marker linked to a quantitative trait locus associated with FRR/CN/SDS resistance, wherein the molecular marker comprises a sequence or antibody to any sequence set forth as any one of SEQ ID NOs:1-4; and (b) determining the presence of FRR/CN/SDS resistance as detection of the molecular marker of step (a) and determining the absence of FRR/CN/SDS resistance as failure to detect the molecular marker of step (a). Said method may further comprise: (a) preparing genomic DNA from the soybean plant, or part thereof; and (b) detecting a molecular marker linked to a quantitative trait locus associated with FRR/CN/SDS resistance, wherein the molecular marker comprises a sequence set forth as any one of SEQ ID NOs:1-4; and (c) determining the presence of FRR/CN/SDS resistance as detection of the molecular marker of step (b) and determining the absence of FRR/CN/SDS resistance as failure to detect the molecular marker of step (b). The step of detecting in the method may comprise a PCR-based assay.

Also provided is a method of reliably and predictably introgres sing FRR/CN/SDS resistance into non-resistant soybean germplasm, the method comprises: (a) identifying one or more nucleic acid markers for marker assisted selection among soybean lines to be used in a soybean breeding program, wherein the nucleic acid markers map to linkage groups G or A2 and wherein the nucleic acid markers are selected from among any of SEQ ID NOs:1, 2 and 4; and (b) introgressing said resistance into said non-resistant soybean germplasm by performing marker-assisted selection. In one example, the soybean germplasm referred in the method is derived from the “Forrest” line, or descendant thereof. Also provided is a plant, seed, or tissue culture produced by the illustrated method, and the plant, seed, or tissue culture is resistant to FRR/CN/SDS infection.

Further provided is a method of positional cloning of a nucleic acid that interacts with SEQ ID NO: 1-4, the method comprising: (a) identifying a first nucleic acid genetically linked to a FRR/CN/SDS resistance locus, wherein the first nucleic acid maps between two markers selected from among any of SEQ ID NOs:1-4; and (b) cloning the first nucleic acid. In one example of said method, the first nucleic acid comprises the Rfs2 gene and the SDS locus. In another example, the first nucleic acid of the method comprises the rhg1 gene. In the general method provided, the method may further comprise hybridizing a second nucleic acid comprising the locus to a genomic library and selecting a clone that hybridizes to the second nucleic acid and comprises a second locus that confers FRR/CN/SDS resistance in a plant. The general method may also comprise hybridizing a second nucleic acid comprising the locus to a genomic library and selecting a clone that hybridizes to the second nucleic acid, wherein the genomic library is selected from the group consisting of a BAC soybean genomic library, a YAC soybean genomic library, and a P1 bacteriophage soybean genomic library. Still, the general method may further comprise identifying overlapping clones. In the general method, the first nucleic acid is amplified by PCR prior to cloning of the first nucleic acid. In one example, the first nucleic acid is proximal to the selected locus, and the general method may further comprise identifying a coding region encoded by the first nucleic acid. In the general method, the FRR/CN/SDS resistance locus corresponds to a nucleic acid selected from among any of SEQ ID NOs: 4, enhanced promoted or terminated by SEQ 5-11 and the encoded proteins SEQ ID NOs: 12-20.

Also provided is a method for producing an antibody that specifically recognizes a FRR/CN/SDS resistance polypeptide, the method comprising: (a) recombinantly or synthetically producing a FRR/CN/SDS resistance polypeptide, or portion thereof; (b) formulating the polypeptide of (a) whereby it is an effective immunogen; (c) administering to an animal the formulation of (b) to generate an immune response in the animal comprising production of antibodies, wherein antibodies are present in the blood serum of the animal; and (d) collecting the blood serum from the animal of (c) comprising antibodies that specifically recognize a FRR/CN/SDS resistance polypeptide. An antibody thereby produced is provided.

Further provided is a method for detecting a level of a FRR/CN/SDS resistance polypeptide, the method comprising (a) obtaining a biological sample having peptidic material; (b) detecting a FRR/CN/SDS resistance polypeptide in the biological sample of (a) by immunochemical reaction with the antibody of claim 55, whereby an amount of a FRR/CN/SDS resistance polypeptide in a sample is determined.

Provided also is a method for identifying a substance that modulates a FRR/CN/SDS resistance polypeptide function, the method comprising: (a) isolating a FRR/CN/SDS resistance polypeptide encoded by the nucleotide sequence of SEQ ID NO:2; a polypeptide encoded by a nucleic acid molecule that is substantially identical to SEQ ID NO:2; a polypeptide having the amino acid sequence of SEQ ID NO:3; a polypeptide that is a biological equivalent of the polypeptide of SEQ ID NO:3; or a polypeptide which is immunologically cross-reactive with an antibody that shows specific binding with a polypeptide of SEQ ID NO:3; (b) exposing the isolated FRR/CN/SDS resistance polypeptide to one or more candidate substances; (c) assaying binding of a candidate substance to the isolated FRR/CN/SDS resistance polypeptide; and (d) selecting a substance that demonstrates selective binding to the isolated FRR/CN/SDS resistance polypeptide.

Still, a method of detecting a nucleic acid molecule that encodes an FRR/CN/SDS resistance polypeptide in a biological sample containing nucleic acid material is provided. The method comprises: (a) hybridizing the nucleic acid molecule of claim 15 under stringent hybridization conditions to the nucleic acid material of the biological sample, thereby forming a hybridization duplex; and (b) detecting the hybridization duplex, whereby a nucleic acid molecule encoding a FRR/CN/SDS resistance polypeptide is detected in the biological sample. In this general method, the nucleic acid molecule that encodes an FRR/CN/SDS resistance polypeptide further comprises a chromosome.

Provided also is a method for identifying soybean sudden death syndrome (SDS) resistance or soybean cyst nematode (SCN) resistance in a plant using a SDS resistance gene, a SCN resistance gene, or DNA segments having homology to a SDS resistance gene or to an SCN resistance gene, the method comprising: (a) probing nucleic acids obtained from the plant with a probe derived from said SDS resistance gene or from said SCN resistance gene or from said DNA segment having homology to said SDS resistance gene or to said SCN resistance gene; and (b) observing hybridization of said probe to said nucleic acids, the presence of said hybridization indicating SDS or SCN resistance in said plant. In this general method, the probe may comprise an isolated and purified nucleic acid molecule encoding a biologically active FRR/CN/SDS resistance polypeptide. In another example, the probe may comprise a nucleotide sequence as set forth in of any of SEQ ID NOs: 1, 2 and 4, or any complementary strand thereof, or any combination thereof.

Further provided is a method for identifying a candidate compound as a modulator of FRR/CN/SDS resistance activity, the method comprising: (a) exposing a cell sample with a candidate compound to be tested, the cell sample containing at least one cell containing a DNA construct comprising a modulatable transcriptional regulatory sequence of an FRR/CN/SDS resistance-encoding nucleic acid and a reporter gene which is capable of producing a detectable signal; (b) evaluating an amount of signal produced in relation to a control sample; and (c) identifying a candidate compound as a modulator of FRR/CN/SDS resistance activity based on the amount of signal produced in relation to a control sample. In this general method, the reporter gene comprises a nucleic acid molecule encoding an FRR/CN/SDS resistance polypeptide. Further, the modulatable transcriptional regulatory sequence in the general method comprises any part of SEQ ID NO:4.

A method of modulating FRR/CN/SDS resistance in a plant is also provided, and the method comprises administering to the plant an effective amount of a substance that modulates expression of an FRR/CN/SDS resistance activity-encoding nucleic acid molecule in the plant to thereby modulate FRR/CN/SDS resistance in the plant. In the general method, the substance that modulates expression of an FRR/CN/SDS resistance activity-encoding nucleic acid molecule comprises a ligand for a regulatory protein that binds a FRR/CN/SDS resistance gene promoter. Further, the FRR/CN/SDS resistance gene promoter in the method comprises the nucleotide sequence of SEQ ID NO: 1, 2 or 4 or functional portion thereof. The general method may further comprises monitoring an insertion point for the construct in the plant genome; and providing for insertion of the construct into the plant genome at a location not associated with the resistance characteristic, the desired characteristic, or both the resistance or the desired characteristic.

Further provided is a method for modulating FRR/CN/SDS resistance in a plant, the method comprising administering to the plant an effective amount of a substance that modulates FRR/CN/SDS resistance polypeptide activity to thereby modulate FRR/CN/SDS resistance in the plant. In one example, the plant is a soybean plant.

A method for providing a resistance trait to a plant is provided, and the method comprises introducing to said plant a construct comprising a nucleic acid sequence encoding an FRR/CN/SDS resistance gene product operatively linked to a promoter, wherein production of the FRR/CN/SDS resistance gene product in the plant provides FRR, FRR, CN or SDS resistance trait to the plant. In this general method, the construct used may further comprise a vector selected from the group consisting of a plasmid vector or a viral vector. In one example, the FRR/CN/SDS resistance gene product in the method comprises a protein having an amino acid sequence of SEQ ID NO:3. In some examples, the nucleic acid sequence of the general method is selected from the group consisting of: (a) a nucleotide sequence set forth as SEQ ID NO:2; (b) a nucleotide sequence substantially similar to SEQ ID NO:2. In one example, the resistance characteristic is nematode resistance, fungal resistance insect resistance or combinations thereof. In one particular example, the nematode resistance is H. glycines resistance. With further specification, the H. glycines resistance may be race 3 H. glycines resistance. In another group of example, the construct of the general method may further comprise another nucleic acid molecule encoding a polypeptide that provides an additional desired characteristic to the plant. The general method may further comprises monitoring an insertion point for the construct in the plant genome; and providing for insertion of the construct into the plant genome at a location not associated with the resistance characteristic, the desired characteristic, or both the resistance or the desired characteristic. In one example, the plant is a soybean plant. In some examples, the FRR resistance in the method is Fusarium spp. resistance. Specifically, the FRR resistance may be Fusarium virguliforme resistance. In one example, the construct of the method further comprises another nucleic acid molecule encoding a polypeptide that provides an additional desired characteristic to the plant. In another example, the method may further comprise monitoring an insertion point for the construct in the plant genome; and providing for insertion of the construct into the plant genome at a location not associated with the resistance characteristic, the desired characteristic, or both the resistance or the desired characteristic. In one specific example, the plant is a soybean plant.

Provided also is a method for producing an antibody or peptide that specifically recognizes a ligand of the FRR/CN/SDS resistance polypeptide, the method comprising: (a) recombinantly or synthetically producing a FRR/CN/SDS resistance polypeptide, or portion thereof; (b) formulating the polypeptide of (a) whereby it is an effective immunogen; (c) administering to an animal the formulation of (b) to generate an immune response in the animal comprising production of antibodies, wherein antibodies are present in the blood serum of the animal; and (d) collecting the blood serum from the animal of (c) comprising antibodies that specifically recognize a FRR/CN/SDS resistance polypeptide. Also provided is an antibody produced thereby.

A method for detecting a level of a FRR/CN/SDS resistance polypeptide ligand is provided. The method comprises (a) obtaining a biological sample having peptidic material; (b) detecting a FRR/CN/SDS resistance polypeptide ligand in the biological sample of (a) by immunochemical reaction with the antibody produced with the method provided herein, whereby an amount of a FRR/CN/SDS resistance polypeptide ligand in a sample is determined.

Further provided is a method for identifying a substance that modulates a FRR/CN/SDS resistance polypeptide ligand function, the method comprising: (a) isolating a FRR/CN/SDS resistance polypeptide encoded by the nucleotide sequence of SEQ ID NO:2; a ligand of the polypeptide encoded by a nucleic acid molecule that is substantially identical to SEQ ID NO:2; a ligand of a polypeptide having the amino acid sequence of SEQ ID NO:3; a ligand of a polypeptide that is a biological equivalent of the polypeptide of SEQ ID NO:3; or a ligand of polypeptide which is immunologically cross-reactive with an antibody that shows specific binding with a ligand of polypeptide of SEQ ID NO:3; (b) exposing the ligand of the isolated FRR/CN/SDS resistance polypeptide to one or more candidate substances; (c) assaying binding of a candidate substance to the isolated ligand of the FRR/CN/SDS resistance polypeptide; and (d) selecting a substance that demonstrates selective binding to the isolated ligand of the FRR/CN/SDS resistance polypeptide.

Provided is also a method of detecting a nucleic acid molecule that encodes a ligand of the FRR/CN/SDS resistance polypeptide in a biological sample containing nucleic acid material, the method comprising: (a) hybridizing the polypeptide molecule of claim 3 under stringent hybridization conditions to material of the biological sample, thereby forming an interaction; and (b) detecting the hybridization duplex, whereby a nucleic acid molecule encoding a FRR/CN/SDS resistance polypeptide is detected in the biological sample.

The nucleic acid molecule in the general methods provided herein may encode a ligand of FRR/CN/SDS resistance polypeptide further comprises a small molecule, peptide or protein.

Also provided is a method of improving the yield of a crop harvested for its biomass which comprises: supplying materials to a field, planting a field with a crop which can be harvested for its biomass, having transformed therein an expressible transgene encoding a Receptor Like Kinase (RLK) embodied in SEQ NO: 1-4, and harvesting the crop having transformed therein the expressible transgene encoding said RLK, for the biomass, wherein the harvested transformed crop has increased biomass yield due to root or leaf size increase relative to a non-transformed crop. In this general method, said gene contains a modified sequence. In one example, said crop in the method is selected from the group consisting of: corn, cotton, brassicas, canola, legumes, soybeans, forage grasses, grasses, wheat or rice.

Provided also are transgenic plant cells, or progeny thereof, formed by transformation of wild type plant cells, said transformed plant cells comprises: 1) an expression cassette having a transcription initiation region functional in the transformed plant cells; 2) a DNA sequence that encodes the RLK in said transformed plant cells; and 3) a transcription termination region functional in said transformed plant cells, wherein said expression cassette imparts increased biomass to transformed plants resulting from the transformed plant cells relative to wildtype plants resulting from the wildtype plant cells. Said at least one of said transcription region and said termination region in the cells provided is not naturally associated with said sequence. Provided herein are cells with said RLK from G. max. In one example, the cells provided have said DNA sequence modified to enhance expression in plant cells. In another example, the cells provided have said DNA sequence encodes the amino acid sequence of SEQ ID NO:3. In one group of cells provided, said transcription initiation region of the cells is constitutive in action. In one group of cells provided, said transcription initiation region is organ specific.

Further provided is a transgenic plant originally formed from nontransgenic plants, or progeny of said transgenic plant, which contains: 1) an expression cassette having a transcription initiation region functional in a plant cell of said transgenic plant; 2) a genetically engineered DNA sequence that encodes a GmRLK18-1 in said plant cells; wherein said transgenic plant evidences detectable increases in said RLK activity when compared to said nontransgenic plants which increases the transgenic plant's biomass relative to that of the nontransgenic plants. In one example, the transgenic plant so provided is a dicot plant. In another example, the transgenic plant provided is a legume. In yet another example, the transgenic plant is Glycine max. In some other example, the transgenic plant is a plant selected from a group consisting of: canola, green vegetables, beans, peas, lettuce, watercress, collard greens, turnip greens, cabbage. In one example, the plant of said transgenic plant is a brassica. In another example, the plant of said transgenic plant is canola.

A method to break the linkage drag between SCN reisitance and seed yield in G. max is also provided herein.

### BRIEF DESCRIPTION OF THE DRAWINGS

FIG. 1 depicts genetic markers for FRR/CN/SDS resistance. Marker map of the genomic region around Rhg1/Rfs2 and the homeolg of Rhg1/Rfs2 with locus ideograms. The gene encoding the RLK was show as a black block arrow. The extent of the p SBHB94 insert of 9.772 kbp in a subclone from BAC 21d09 that was used for soybean transformations was shown as a blue arrow. The marker TMD1 amplified a fragment from both homeologs of 303±15 bp and 362 bp. Sequence coordinates were from [12]. FIG. 1A shows the marker map of the genomic region around Rhg1/Rfs2 (Lg G; chromosome 18) with locus ideograms. Sequence coordinates were from the susceptible cultivar A3244 (Hague et al 2007 U.S. Pat. No. 7,154,021; Ruben et al. 2006; Mol Genet Genom 276:503-516). The gene encoding the RLK was shown as a black block arrow. The genes encoding the laccase and antiporter were shown as opposite white block arrows. All other genes were shown as grey block arrows. Locations of overlapping BAC clones B73P06 (SEQ ID NO; 4) and B21d09 that both encoded the Rhg1/Rfs2 locus (Lg G; chromosome 18) are shown below the ideogram. FIG. 1B shows the BAC clones B73P06 that encoded the Rhg1/Rfs2 locus (Lg G; chromosome 18). The gene encoding the RLK was shown as a black block arrow. The genes encoding the laccase and antiporter were shown as opposite white block arrows. All other genes were shown as grey block arrows. Sequence coordinates were from the complete sequence of the BAC derived from resistant cultivar Forrest (SEQ NO. 4). The extent of the insert of p SBHB94, the 9,772 kbp subclone from BAC B21d09 that was used for soybean transformations was shown as a blue arrow (SEQ No. 2). FIG. 1C showed a syntenic homeolog of Rfs2/rhg1 found in the sequence of BAC H38F23 from Lg B1 (chromosome 11). The homeolog of the gene encoding the RLK was show as a black block arrow. The homeologs of the genes encoding the laccase and antiporter were shown as opposite white block arrows. All other syntenic genes were shown as grey block arrows. The marker TMD 1 amplified a fragment from Rfs2/rhg1 of 303±15 bp (resistant allele was the smaller) and of 362 bp from a syntenic homeolog of Rfs2/rhg1 found in the sequence of BAC H38F23 from Lg B1 (chromosome 11).

FIG. 2 depicts Phenotypes of transgenic plants expressing FRR/CN/SDS resistance from p SBHB94 DNA to Fusarium virguliforme and Heterodera glycines caused by a receptor like kinase found at Rfs2/Rhg1 locus as transgene in primary transgenic lines (cv ‘X5’). F. virguliforme was used at 10⁴ cfu per cm³ of soil. Specifically, resistance to Fusarium virguliforme and Heterodera glycines caused by the Forrest allele of a receptor like kinase (GmRLK18-1-a) found at the Rfs2/Rhg1 locus as transgene in primary transgenic lines (cv ‘X5’). The experiment was carried out on 3 separate occasions. Leaf scorch was scored as DS at 7, 14, 21, 28, 35, 42, 49 and 56 dai. Derived crossed lines (×WENIL35) and cultivars (×EF2) were included in runs 2 and 3. Panel A & B shows stable soybean transgenics with and without the 10 kbp Rhg1/Rfs2 subclone at 21 dai. Panel C shows the RLK transgene reduced root rot at 28 dai. Panel D shows leaf symptoms at 28 dai. Panel E shows plants at 56 dai where X5 is senescent with erect petioles and X5::RLK is still green and filling pods. Panel F shows selected leaflets at 28 dai with a 1-9 range in DS scores. Panels A-H show the SDS assays. F. virguliforme was used at 104 cfu per cm3 of soil. The experiment was carried out on 3 separate occasions. Leaf scorch was scored as DS at 7, 14, 21, 28, 35, 42, 49 and 56 dai (days after infestation). Derived crossed lines (X5::GmRLK18-1-a×WENIL35 and cultivars X5:: GmRLK18-1-a×WENIL35×EF2) were included in runs 2 and 3. Panel A & B shows stable soybean transgenics with and without the 9.772 kbp GmRLK18-1 (Rhg1/Rfs2) subclone pSBHB94 at 21 dai. Panel C shows the GmRLK18-1-a transgene reduced root rot at 28 dai. Panel D shows leaf symptoms at 28 dai. Panel E shows plants at 56 dai where X5 is senescent with abscission of leaflets from erect petioles and X5::GmRLK18-1-a is still green and filling pods. Panel F shows selected leaflets at 28 dai with a 1-9 range in DS scores arranged in order of severity from bottom left to top right. Panels G-J show the SCN assays. Panel G shows SCN arrested in development by the RLK in X5 transgenics. Panel H shows SCN arrested in development by the Rhg1-a allele in resistant NIL 34-23. Panel I shows normal SCN development in the susceptible X5. Panel J shows normal SCN development in susceptible NIL 34-3. Panel K shows a GmRLK18-1-a transgenic plant of cultivar X5 that was defoliated by insect herbivory in the field. Panel L shows an non-transgenic X5 plant with much less leaf area loss in 2010 field trials

FIG. 3 shows soybean transgenic plants expressed the mRNA and protein from the RLK at the Rfs2/Rhg1 locus. Soybean transgenic plants expressed the mRNA and protein from the Forrest allele of GmRLK18-1, the RLK at the Rfs2/Rhg1 locus. Panel A shows PCR from leaf samples of progeny plants derived from a primary transgenic event 6B3-7D2(1) with TMD1 primers. Lanes contain transgenic plants 1 to 13. The arrow shows the double band for Gm18RLK-1-a positive sample at 314 bp for lines 1, 3-5, 7, 8, 10 and 12. M was the marker; H was the no DNA (water) control; P was the Rhg1 plasmid pSBHB94; X5 was the control plant. Panel B shows PCR from cDNA leaf samples of sixteen transgenic lines derived from event 6B3-7D2(1) with HRM primers and Taqman detection of mRNA by RT-PCR. Detection of the SNP polymorphism at position 2070 in the LRR region of rhg1 using an allelic discriminatory assay. A Fam labeled probe was used for the detection of resistant haplotypes 1 and 2 (red) and Hex labeled probe for the detection of susceptible haplotypes 2, 3 and 4 (blue). A total of 16 individuals were selected for the analysis. For HRM green lines are from transgenic plants. Red melt curve was a resistant control blue line was a susceptible control. Panel C shows a Western of a 2D gel from roots of a transgenic plant probed with the anti-RLK peptide antibody. An alloprotein at pI 8.42 and 92.41 kDa was found in the non transgenic cv X5 but the presence of the Forrest alloprotein at pI 8.44 and 92.39 kDa was found in transgenic plants derived from event 6B3-7D2(1) expressing GmRLK18-1-a. GmRLK18-1 was shown to be a very low abundance protein impossible to visualize without immunostaining. FIG. 3A shows soybean transgenic plants expressed the mRNA and protein from the Forrest allele of GmRLK18-1, the RLK at the Rfs2/Rhg1 locus. Panel A shows PCR from leaf samples of progeny plants derived from a primary transgenic event 6B3-7D2(1) with TMD1 primers. Lanes contain transgenic plants 1 to 13. The arrow shows the double band for Gm18RLK-1-a positive sample at 314 bp for lines 1, 3-5, 7, 8, 10 and 12. M was the marker; H was the no DNA (water) control; P was the Rhg1 plasmid pSBHB94; X5 was the control plant. Panel B shows PCR from cDNA leaf samples of sixteen transgenic lines derived from event 6B3-7D2(1) with HRM primers and Taqman detection of mRNA by RT-PCR. Detection of the SNP polymorphism at position 2070 in the LRR region of rhg1 using an allelic discriminatory assay. A Fam labeled probe was used for the detection of resistant haplotypes 1 and 2 (red) and Hex labeled probe for the detection of susceptible haplotypes 2, 3 and 4 (blue). A total of 16 individuals were selected for the analysis. For HRM green lines are from transgenic plants. Red melt curve was a resistant control blue line was a susceptible control. Panel C shows a Western of a 2D gel from roots of a transgenic plant probed with the anti-RLK peptide antibody. An alloprotein at pI 8.42 and 92.41 kDa was found in the non transgenic cv X5 but the presence of the Forrest alloprotein at pI 8.44 and 92.39 kDa was found in transgenic plants derived from event 6B3-7D2(1) expressing GmRLK18-1-a. GmRLK18-1 was shown to be a very low abundance protein impossible to visualize without immunostaining.

FIG. 4: Far-Western analysis of soybean root proteins at 24 dap (10 dai) probed with the LRR domain of GmRLK18-1. Far-Western analysis of soybean root proteins at 24 dap (10 dai) probed with the LRR domain of GmRLK18-1. Panel (A): Shown is a portion of a 2D gel (14.4-21.5 KDa; 7.5-10.0 pI) from 34-23 (resistant) SCN inoculated total root proteins with spots visualized with silver staining. Panel (B): Proteins transferred to a membrane and probed with purified GmRLK18-1 LRR domain and 6× his-RHG1. Anti-His-HRP was used as the secondary probe. The single spot identified (arrowed) was excised from the duplicate gel and analyzed by Q-TOF (MS-MS) to identify a cyclophilin as a GmRLK18-1 LRR domain interacting partner. FIG. 4A: Shown is a portion of a 2D gel (14.4-21.5 KDa; 7.5-10.0 pI) from 34-23 (resistant) SCN inoculated total root proteins with spots visualized with silver staining. Panel B: Proteins transferred to a membrane and probed with purified GmRLK18-1 LRR domain and 6× his-RHG1. Anti-His-HRP was used as the secondary probe. The single spot identified (arrowed) was excised from the duplicate gel and analyzed by Q-TOF (MS-MS) to identify a cyclophilin as a GmRLK18-1 LRR domain interacting partner.

FIG. 5: Far-Western analysis of soybean root proteins at 42 dap (28 dai) probed with the LRR domain of GmRLK18-1. Far-Western analysis of soybean root proteins at 42 dap (28 dai) probed with the LRR domain of GmRLK18-1. Panel (A): Shown is a whole 2D gel (6.5-116.0 KDa; 3.0-10.0 pI) from 34-23 (resistant) SCN inoculated total root proteins with spots visualized with silver staining. Panel (B): Proteins transferred to a membrane and probed with purified GmRLK18-1 LRR domain and 6× his-RHG1. Anti-His-HRP was used as the secondary probe. The single spot identified (arrowed) was excised from the duplicate gel and analyzed by Q-TOF (MS-MS) to identify methionine synthetase (GI:33325957) at 84.2 KDa and pI 5.93 as a GmRLK18-1 LRR domain interacting partner. The other 3 proteins were of higher abundance and so not likely to be specific interactions. Panel A: Shown is a whole 2D gel (6.5-116.0 KDa; 3.0-10.0 pI) from 34-23 (resistant) SCN inoculated total root proteins with spots visualized with silver staining. Panel B: Proteins transferred to a membrane and probed with purified GmRLK18-1 LRR domain and 6× his-RHG1. Anti-His-HRP was used as the secondary probe. The single spot identified (arrowed) was excised from the duplicate gel and analyzed by Q-TOF (MS-MS) to identify methionine synthetase (GI:33325957) at 84.2 KDa and pI 5.93 as a GmRLK18-1 LRR domain interacting partner.

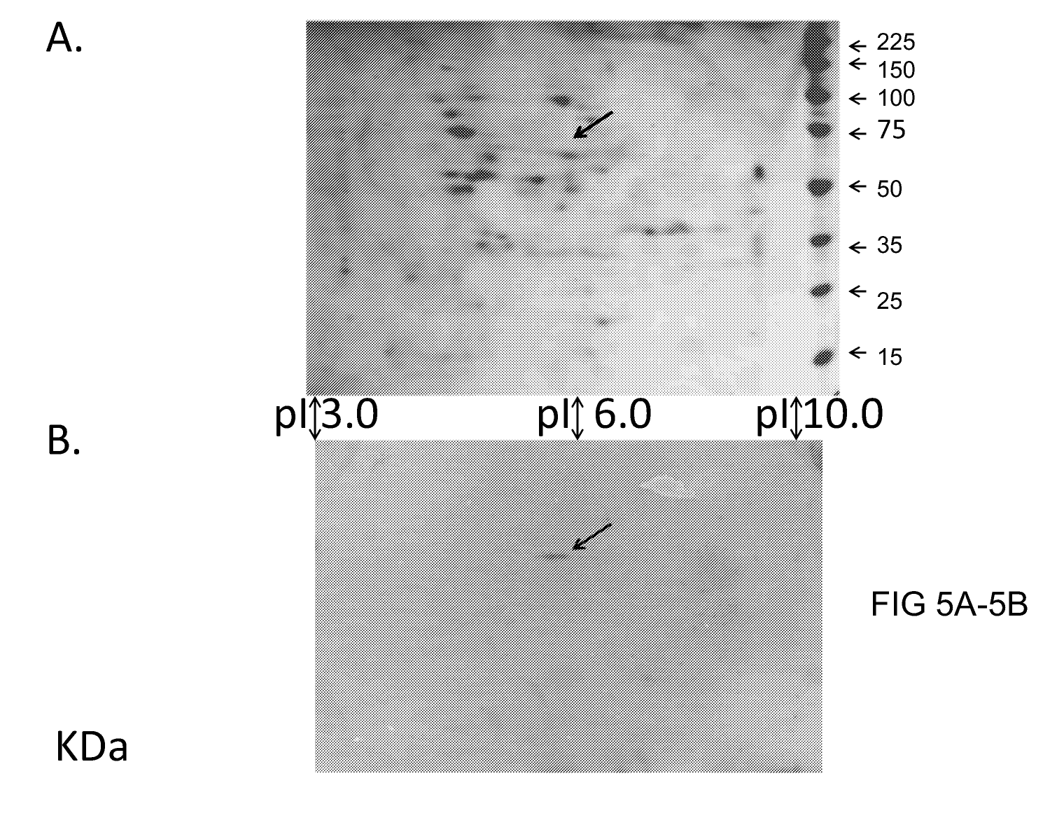

FIG. 6: Negative growth effects of the transforming BACs B73P06 and positive effects of BAC H38F23 on the growth of a brassica, Arabidopsis thaliana.

### DETAILED DESCRIPTION OF THE INVENTION

Disclosed herein is the identification of DNA markers that are genetically linked to the FRR/CN/SDS resistance loci of Forrest. Further disclosed are purified and isolated FRR, CN or SDS resistance genes, proximal sequences to FRR/CN/SDS resistance genes, and FRR/CN/SDS resistance-related genes.

Table of Abbreviations

Ab—antibody
BAC—bacterial artificial chromosome
bp—base pair
CLE—CLAVATA 3/ESR(embryo-surrounding region)-related (‘CLE’)
peptide family
FAM—6-carboxyfluorescein
FI—female index of parasitism
indel—a nucleotide insertion or deletion
QTL—quantitative trait loci
RAPD—random amplified polymorphic DNA
RFLP—restriction fragment length polymorphism
rhg1—genetic locus conferring resistance to Heterodera glycines
RIL—recombinant inbred line
SCN—soybean cyst nematode
SDS—sudden death syndrome
SNP—single nucleotide polymorphism
SSR—microsatellite
TAMRA—6-carboxy-N,N,N′5N′ tetrachlorofluorescein
TET—6-carboxy-4,7,2′,7′, tetrachlorofluorescein

### List Of Sequences And Tables:

SEQ ID NO.: 1. Sequence of the mRNA coding region of the Rfs2/rhg1 RLK gene SEQ ID NO.: 2. Promoter, gene and terminator region of the RLK on p SBHB94 used in transgenic soybean plants: GenBank 9772 bp DNA linear PLN 30-NOV-2011 HQ008939.1 GI:330722945.

SEQ ID NO.: 3. Receptor like kinase protein coding region of the Rfs2/rhg1 gene: GenBank gi|300519109|gb|AF506517.2|.

SEQ ID NO.: 4. BAC B73P06 complete sequence from resistant cultivar Forrest: 82157 bp DNA linear GenBank PLN 22-NOV-2011 JN597009.1 GI:357432827.

SEQ ID NOS.: 5-11. Promoters, genes and terminator regions of the proteins encoded by BAC B73P06 complete sequence most closely linked to the Rfs2/rhg1 gene.

SEQ ID NOS.: 12-20. Protein coding regions of the of the proteins encoded by BAC B73P06 complete sequence linked to the Rfs2/rhg1 gene.

SEQ ID NO.: 21. Peptide sequence used to generate a specific antibody against the leucine rich repeat domain of the receptor like kinase encoded by the Rfs2/rhg1 gene and to test the dissociation constant (Kd) of dimerization with the purified leucine rich repeat domain of the receptor like kinase encoded by the Rfs2/rhg1 gene.

SEQ ID NO.: 22. Protein coding sequence of the cyclophilin that binds with the purified leucine rich repeat domain of the receptor like kinase encoded by the Rfs2/rhg1 gene: GenBank gi 17981611 (gb AAL51087.1).

SEQ ID NO.: 23. Protein coding sequence of the methionine synthase that binds with the purified leucine rich repeat domain of the receptor like kinase encoded by the Rfs2/rhg1 gene. GenBank gi:33325957.

SEQ ID NOS.: 24-32. Peptide sequences used to test the Kd of ligand binding with the purified leucine rich repeat domain of the receptor like kinase encoded by the Rfs2/rhg1 gene.

SEQ ID NO.: 33. Complete sequence of BAC H38F23 from Lg B1 (chromosome 11) that contained a syntenic homeolog of Rfs2/rhg1 and 11 linked genes.

SEQ ID NOS.: 34-39. Protein coding regions of the of the proteins encoded by BAC H38F23 from Lg B1 (chromosome 11; SEQ ID NO; 33) that contained a syntenic homeolog of Rfs2/rhg1.

Table 1. Amino acids and their codons.

Table 2. Nucleotide and protein differences between the resistant genes and proteins in a resistant soybean cultivar compared to two susceptible soybean cultivars.

Table 3. Association of mean root growth in NILs and transgenic lines with resistance to pests. Panels A (NILs) and B (transgenics) shows root growth, SCN and SDS responses in greenhouse grown seedlings at 28 days after germination with SCN infestations or F. virguliforme infestations. Female index (FI) was a percentage of cysts of Hg Type 0 found compared to a susceptible line. Disease severity (DS) was a 1-9 scale for the leaf scorch caused by F. virguliforme characteristic of sudden death syndrome (SDS). Panel C shows the percent insect incidence, defoliation by herbivorous insects and the consequent loss of biomass at harvest as mean dry weight per plant for field grown plants.

Table 4. Ligands that bind in vitro to purified LRR domain of the RLK at rhg1/Rfs2.

Table 5. Proteins altered in abundance by more than 2 fold in soybean roots infested by Fusarium virguliforme containing the resistance allele of the RLK at Rfs2.

The isolated and purified polynucleotide sequences disclosed herein can thus be used in a variety of applications pertaining to breeding and engineering soybeans having SCN and SDS resistance. For example, the isolated polynucleotides disclosed herein can be used in position-based or homology-based cloning of additional FRR/CN/SDS resistance genes, including regulatory elements; in gene structure determination; in studies of genome organization and gene expression; in gene complementation experiments; in the isolation of additional DNA markers for gene manipulation and molecular marker assisted breeding; and in plant transformation and the production of transgenic plants.

The present invention also pertains to a soybean plant and methods of producing the same, which is resistant to soybean cyst nematodes (SCN). In one embodiment, the method comprises stable transformation of a plant with an rhg1 gene, disclosed herein. In another embodiment, the method comprises introgression in soybean of a trait enabling the plant to resist soybean cyst nematode (SCN) infestation. Additionally, the present invention relates to method of precise and accurate introgression of the genetic material conferring SCN resistance from one or more parent plants into the progeny.

The present invention also pertains to a soybean plant and methods of producing the same, which is resistant to soybean sudden death syndrome (SDS). In one embodiment, the method comprises stable transformation of a plant with an rhg1 gene, disclosed herein. In another embodiment, the method comprises introgression of the genetic material conferring SDS resistance from one or more parent plants into the progeny with precision and accuracy.

The invention differs from present technology in several regards. In one aspect, the present invention provides the first disclosure of the rhg1 gene sequence, thereby enabling transgenic approaches for providing FRR/CN/SDS resistance. Further, the present invention provides a non-electrophoretic selection assay using nucleotide sequences of FRR/CN/SDS resistance gene alleles. The disclosed nucleotide sequences of FRR/CN/SDS resistance genes and associated genetic markers provide means for easily selecting resistant cultivars, for assembling many resistance genes in a single cultivar, for combining resistance genes in novel combinations, for identifying genes that confer resistance in new cultivars, and for predicting resistance in cultivars. The invention is used to improve selection for SDS and SCN resistance in soybean in breeding programs.

### I. Traits

The term “phenotype” or “trait” each refer to any observable property of an organism, produced by the interaction of the genotype of the organism and the environment. A phenotype can encompass variable expressivity and penetrance of the phenotype. Exemplary phenotypes include but are not limited to a visible phenotype, a physiological phenotype, a susceptibility phenotype, a cellular phenotype, a molecular phenotype, and combinations thereof. Preferably, the phenotype is related to FRR/CN/SDS resistance. The term “susceptibility phenotype” refers to an increased capacity or risk for displaying a phenotype, i.e. a susceptibility to FRR/CN/SDS infection.

The term “complex trait” as used herein refers to a trait that is not inherited as predicted by classical Mendelian genetics. A complex trait results from the interaction of multiple genes, each gene contributing to the phenotype. Complex traits can be continuous or show threshold penetrance. In the field, FRR/CN/SDS resistance is inherited as a complex trait.

The term “quantitative trait” is a complex trait that can be assessed quantitatively. Quantitation entails measurement of a trait across a continuous distribution of values. FRR/CN/SDS resistance is a quantitative trait.

The term “FRR/CN/SDS resistance” or “FRR/CN/SDS resistance trait” as used herein refers to a cellular or organismal capacity for resistance to nematode or fungal infection, or both. Preferably, the nematode resistance is Heterodera glycines (the organism that causes SCN in soybeans) resistance, even more preferably race 3 Heterodera glycines resistance. The fungal resistance is preferably Fusarium virguliforme (the organism that causes SDS in soybeans)-infection resistance. SCN resistance can be assayed in the field or in the greenhouse by methods known in the art, including but not limited to determination of an SCN index of parasitism as disclosed in Example 2, Meksem et al. (1999), and U.S. Pat. No. 6,096,944. SDS resistance can be scored by determination of disease incidence, disease severity, and disease index values as disclosed in Hnetkovsky et al. (1996) Crop Sci 36(2):393-400, Njiti et al.

(1996) Crop Sci 36:1 165-1170; and Matthews et al. (1991).

The term “FRR/CN/SDS resistance” is used herein for convenience to describe traits, transgenic plants, polynucleotides, and polypeptides of the present invention. Therefore, the resistance characteristic conveyed by the polynucleotides and polypeptides of the present invention refers to any resistance characteristic as set forth herein and as would be apparent to one of ordinary skill in the art after reviewing the disclosure of the present invention.

The term “molecular phenotype” refers to a detectable feature of molecules in a cell or organism. Exemplary molecular phenotypes include but are not limited to a presence of a genetic marker nucleotide sequence, a presence of a FRR/CN/SDS resistance gene sequence, a level of gene expression, a splice selection, a level of protein, a protein type, a protein modification, a level of lipid, a lipid type, a lipid modification, a level of carbohydrate, a carbohydrate type, a carbohydrate modification, and combinations thereof. Methods for observing, detecting, and quantitating molecular phenotypes are well known to one skilled in the art. See Sambrook et al., eds. (1989) Molecular Cloning, Cold Spring Harbor Laboratory Press, Cold Spring Harbor, N.Y., N.Y.; by Silhavy et al. (1984) Experiments with Gene Fusions, Cold Spring Harbor Laboratory Press, Cold Spring Harbor, N.Y., N.Y.; by Ausubel et al. (1992) Current Protocols in Molecular Biology, John Wylie and Sons, Inc. New York, N.Y.; Landgren et. al. (1988) Science 242:229-237; Bodanszky, et al. (1976) Peptide Synthesis, John Wiley and Sons, Second Edition, New York, N.Y.; Harlow and Lane (1988) Antibodies: A Laboratory Manual, Cold Spring Harbor Laboratory Press, Cold Spring Harbor, N.Y.; Ochman et al. (1990) in PCR protocols: a Guide to Methods and Applications, Innis et al. (eds.), pp. 219-227, Academic Press, San Diego, Calif.; Koduri and Poola (2001) Steroids 66(1):17-23; Regan et al. (2000) Anal Biochem 286(2):265-276; U.S. Pat. Nos. 6,096,555; 5,958,624; and 5,629,158.

### II. Genetic Mapping

For genetic mapping, a representative population was generated as in Example 1. To detect genomic regions associated with resistance to SCN and resistance to SDS, the RILs were classified as Essex type or Forrest type for each marker. In some cases, SCN susceptibility and resistance was quantitatively determined according to a SCN female index (F1) of parasitism (Meksem, 1999) as described in Example 2. Markers were compared with FRR, CN or SDS response scores by the F-test in analysis of variance (ANOVA) done with SAS (SAS Institute Inc., Cary, N.C., 1988). The probability of association of each marker with each trait was determined and a significant association was declared if P.ltoreq.0.05 (unless noted otherwise in the text) since the detection of false associations is reduced in isogenic lines (Landers & Botstein (1989) Genetics 121:185-199; Paterson et al. (1990) Genetics 124:735-742).

Selected pairs of markers were analyzed by the two-way ANOVA using the general linear model (PROC GLM) procedure to detect non-additive interactions between the unlinked QTL (Chang et al. (1996) Crop Sci 36:965-971) or Epistat (Chase et al. (1997) Theor Appl Genet. 94:724-730). Non-additive interactions between markers which were significantly associated with FRR/CN/SDS response were excluded when P.gtoreq.0.05. Selected groups of markers were analyzed by multi-way ANOVA to estimate joint heritabilities for traits associated with multiple QTL. Joint heritability was determined from the R² term for the joint model in multi-way ANOVA.

Mapmaker-EXP 3.0 (Lander et al. 1987) was used to calculate map distances (cM, Haldane units) between linked markers and to construct a linkage map including traits as genes. The RIL (recombinant inbred line) and F₃ self genetic models were used. The log₁₀ of the odds ratio (LOD) for grouping markers was set minimally at 2.0, and maximum distance was set at 30 cM. Conflicts were resolved in favor of the highest LOD score after checking the raw data for errors. Marker order within groups was determined by comparing the likelihood of many map orders. A maximum likelihood map was computed with error detection. Trait data were used for QTL analysis (Webb et al. 1995; Chang et al. 1997). The data were subjected to ANOVA (SAS Institute Inc., Cary, N.C.) with mean separation by LSD (Gomez and Gomez (1984). Graphs were constructed by Quattro Pro version 5.0 (Novell Inc., Orem, Utah).

### III. Nucleotide Sequences of FRR/CN/SDS Resistance Genes and Associated Genetic Markers

The nucleic acid molecules provided by the present invention include the isolated nucleic acid molecules of SEQ ID NOs: 1, 2, 4 and 32 sequences substantially similar to sequences of SEQ ID NOs: 1, 2, 4 and 32 conservative variants thereof, plant-expressible variants thereof, subsequences and elongated sequences thereof, complementary DNA molecules, and corresponding RNA molecules. The present invention also encompasses genes, cDNAs, promoters, chimeric genes, and vectors comprising disclosed FRR/CN/SDS resistance gene and FRR/CN/SDS resistance gene marker nucleic acid sequences.

### III.A. General Considerations

The term “nucleic acid molecule” refers to deoxyribonucleotides or ribonucleotides and polymers thereof in either single- or double-stranded form. Unless specifically limited, the term encompasses nucleic acids containing known analogues of natural nucleotides that have similar properties as the reference natural nucleic acid. Unless otherwise indicated, a particular nucleotide sequence also implicitly encompasses conservatively modified variants thereof (e.g. degenerate codon substitutions), complementary sequences, subsequences, elongated sequences, as well as the sequence explicitly indicated. The terms “nucleic acid molecule” or “nucleotide sequence” can also be used in place of “gene”, “cDNA”, or “mRNA”. Nucleic acids can be derived from any source, including any organism.

The term “isolated”, as used in the context of a nucleic acid molecule, indicates that the nucleic acid molecule exists apart from its native environment and is not a product of nature. An isolated DNA molecule can exist in a purified form or can exist in a non-native environment such as a transgenic host cell.

The term “purified”, when applied to a nucleic acid, denotes that the nucleic acid is essentially free of other cellular components with which it is associated in the natural state. Preferably, a purified nucleic acid molecule is a homogeneous dry or aqueous solution. The term “purified” denotes that a nucleic acid or protein gives rise to essentially one band in an electrophoretic gel. Particularly, it means that the nucleic acid is at least about 50% pure, more preferably at least about 85% pure, and most preferably at least about 99% pure.

The term “substantially identical”, in the context of two nucleotide or amino acid sequences, can also be defined as two or more sequences or subsequences that have at least 60%, preferably 80%, more preferably 90-95%, and most preferably at least 99% nucleotide or amino acid sequence identity, when compared and aligned for maximum correspondence, as measured using one of the following sequence comparison algorithms (described herein below under the heading Nucleotide and Amino Acid Sequence Comparisons) or by visual inspection. Preferably, the substantial identity exists in nucleotide sequences of at least 50 residues, more preferably in nucleotide sequence of at least about 100 residues, more preferably in nucleotide sequences of at least about 150 residues, and most preferably in nucleotide sequences comprising complete coding sequences.

In one aspect, polymorphic sequences can be substantially identical sequences. The term “polymorphic” refers to the occurrence of two or more genetically determined alternative sequences or alleles in a population. An allelic difference can be as small as one base pair.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogenous population of nucleic acid molecules. “Target sequence” is synonymous with “test sequence”.

A preferred nucleotide sequence employed for hybridization studies or assays includes probe sequences that are complementary to or mimic at least an about 14 to 40 nucleotide sequence of a nucleic acid molecule of the present invention. Preferably, a probe comprises 14 to 20 nucleotides, or even longer where desired, such as 30, 40, 50, 60, 100, 200, 300, or 500 nucleotides or up to the full length of any of SEQ ID NOs: 1, 2, 4 and 32. Such fragments can be readily prepared by, for example, directly synthesizing the fragment by chemical synthesis, by application of nucleic acid amplification technology, or by introducing selected sequences into recombinant vectors for recombinant production. The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA). The phrase “binds substantially to” refers to complementary hybridization between a probe nucleic acid molecule and a target nucleic acid molecule and embraces minor mismatches that can be accommodated by reducing the stringency of the hybridization media to achieve the desired hybridization. Probe sequences can also hybridize specifically to duplex DNA under certain conditions to form triplex or other higher order DNA complexes. The preparation of such probes and suitable hybridization conditions are well known in the art.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2, Elsevier, N.Y., N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.15 M NaCl at 65° C. An example of stringent wash conditions is 15 minutes in 0.2×SSC buffer at 65° C. (See Sambrook et al., 1989) for a description of SSC buffer). Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the present invention: a probe nucleotide sequence preferably hybridizes to a target nucleotide sequence in 7% sodium dodecyl sulfate (SDS), 0.5 M NaPO₄, 1 mM EDTA at 50° C. followed by washing in 2×SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 1×SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 0.5×SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 0.1×SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, are biologically functional equivalents; or are immunologically cross-reactive. These terms are defined further under the heading FRR/CN/SDS Resistance Polypeptides herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions (Table 1) wherein the third position of one or more selected (or all) codons is substituted with mixed-base and/or deoxyinosine residues (Batzer et al. (1991) Nucleic Acid Res. 19:5081; Ohtsuka et al. (1985) J Biol Chem 260:2605-2608; Rossolini et al. (1994) Mol Cell Probes 8:91-98).

The term “plant-expressible variant” means a substantially similar sequence that has been modified to comprise a coding sequence (nucleotide sequence) can be efficiently expressed by plant cells, tissue and whole plants. The art understands that a plant-expressible coding sequence has a GC composition consistent with good gene expression in plant cells, a sufficiently low CpG content so that expression of that coding sequence is not restricted by plant cells, and codon usage which is consistent with that of plant genes. Where it is desired that the properties of the plant-expressible FRR/CN/SDS resistance gene are identical to those of the naturally occurring FRR/CN/SDS resistance gene, the plant-expressible homolog will have an identical coding sequence or a substantially identical coding sequence.

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the present invention encompass oligonucleotides of sufficient length and appropriate sequence so as to provide initiation of polymerization on a nucleic acid molecule of the present invention.

The term “elongated sequence” refers to an addition of nucleotides (or other analogous molecules) incorporated into the nucleic acid. For example, a polymerase (e.g., a DNA polymerase), e.g., a polymerase that adds sequences at the 3′ terminus of the nucleic acid molecule can be employed to prepare an elongated sequence. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequence”, as used herein, indicates two nucleotide sequences that comprise anti-parallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. A particular example of a complementary nucleic acid segment is an antisense oligonucleotide.

The present invention further includes vectors comprising the disclosed FRR/CN/SDS resistance gene sequences, including plasmids, cosmids, and viral vectors. The term “vector”, as used herein refers to a DNA molecule having sequences that enable its replication in a compatible host cell. A vector also includes nucleotide sequences to permit ligation of nucleotide sequences within the vector, wherein such nucleotide sequences are also replicated in a compatible host cell. A vector can also mediate recombinant production of an FRR/CN/SDS resistance gene polypeptide, as described further herein below.

Nucleic acids of the present invention can be cloned, synthesized, recombinantly altered, mutagenized, or combinations thereof. Standard recombinant DNA and molecular cloning techniques used to isolate nucleic acids are well known in the art. Exemplary, non-limiting methods are described by Sambrook et al., eds., 1989; by Silhavy et al., 1984; by Ausubel et al., 1992; and by Glover, ed. (1985) DNA Cloning: A Practical Approach, MRL Press, Ltd., Oxford, United Kingdom. Site-specific mutagenesis to create base pair changes, deletions, or small insertions are also well known in the art as exemplified by publications, see e.g., Adelman et al., (1983) DNA 2:183; Sambrook et al. (1989).

Nucleotide sequences of the present invention can detected, subcloned, sequenced, and further evaluated by any measure well known in the art using any method usually applied to the detection of a specific DNA sequence including but not limited to dideoxy sequencing, PCR, oligomer restriction (Saiki et al., Bio/Technology 3:1008-1012 (1985), allele-specific oligonucleotide (ASO) probe analysis (Conner et al. (1983) Proc Natl Acad Sci USA 80:278), and oligonucleotide ligation assays (OLAs) (Landgren et. al. (1988) Science 241:1007). Molecular techniques for DNA analysis have been reviewed (Landgren et. al. (1988) Science 242:229-237).

TABLE 1
Table of Functionally Equivalent Codons
for Amino Acids.

Codons Alanine Ala A GCA GCC GCG GCU Cysteine Cys
C UGC UGU Aspartic Acid Asp D GAC GAU Glumatic
acid Glu E GAA GAG Phenylalanine Phe F UUC UUU
Glycine Gly G GGA GGC GGG GGU Histidine His H CAC
CAU Isoleucine Ile I AUA AUC AUU Lysine Lys K AAA
AAG Leucine Leu L UUA UUG CUA CUC CUG CUU
Methionine Met M AUG Asparagine Asn N AAC AAU
Proline Pro P CCA CCC CCG CCU Glutamine Gln Q
CAA CAG Arginine Arg R AGA AGG CGA CGC CGG
CGU Serine Ser S ACG AGU UCA UCC UCG UCU
Threonine Thr T ACA ACO ACG ACU Valine Val V GUA
GUC GUG GUU Tryptophan Trp W UGG Tyrosine Tyr Y
UAC UAU

### III.B. Genetic Markers

The term “genetic marker”, as used herein generally refers to a genetic locus, a phenotype conferred by locus, or a nucleotide sequence residing at a locus, wherein the locus is genetically linked to a trait of interest. The term “genetically linked” as used herein refers to two or more loci that are predictably inherited together during random crossing or intercrossing. Quantitative linkage analysis is further described in the section Genetic Mapping herein above. Preferably, genetically linked loci are less than about 10 cM apart, more preferably less than about 5 cM apart, and even more preferably less than about 1 cM apart. Optimally, the genetic marker and the gene conferring a trait of interest comprise the same or overlapping nucleotide sequence.

An embodiment of the present invention comprises genetic markers associated with SCN resistance and SDS resistance that are isolatable from soybeans, and which are free from total genomic DNA. Disclosed herein are sequences of SNP and indel markers mapped in soybean to the chromosomal segments carrying rhg1, Rfs2 and other SDS loci on molecular linkage group G (Rfs1 and Rfs3). Representative markers for FRR/CN/SDS resistance are set forth as SEQ ID NOs: 1, 3, 4. Representative corresponding markers for FRR/CN/SDS susceptibility are set forth in Table 2.

### III.C. FRR/CN/SDS Resistance Genes

The term “gene” refers broadly to any segment of DNA associated with a biological function. A gene encompasses sequences including but not limited to a coding sequence, a promoter region, a cis-regulatory sequence, a non-expressed DNA segment, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

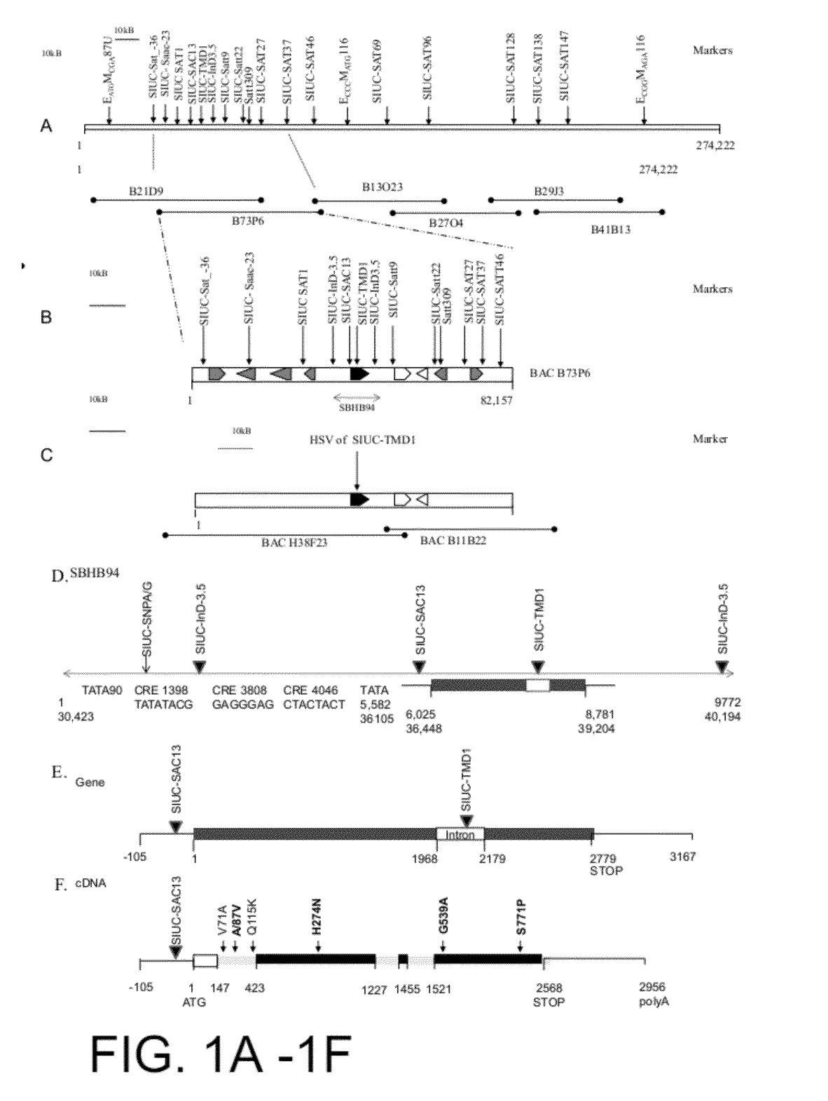

The term “gene” thus includes an isolated soybean rhg1, Rfs2 and SDS resistance gene as disclosed herein (FIG. 1; SEQ 1-4). The gene is capable of conveying Heterodera glycines-infestation resistance or Fusarium virguliforme-infection resistance to a non-resistant soybean germplasm, the gene located within a quantitative trait locus mapping to linkage group G (chromosome 18) and mapped by genetic markers of FIG. 1 and Table 2, said gene located along said quantitative trait locus between said markers. Positional cloning methods were used to isolate genomic sequences in the chromosomal regions of Forrest that confers FRR/CN/SDS resistance, as further described in Example 4. Specifically, rhg1 and Rfs2 sequences were derived from BAC clones 21D9 and 73P6 of the Forrest BamHI or HindIII BAC libraries (Meksem et al., 2000). Preferably, the gene comprises the nucleotide sequence set forth as SEQ ID: 2. BLASTP analysis of the conceptual translation of the rhg1 and Rfs2 gene, set forth as SEQ ID: 3 shows high homology to the T46070 GenBank entry described as hypothetical protein T18N14.120 from Arabidopsis thaliana, high homology to the rice Xa21 disease resistance gene encoding a leucine-rich repeat protein, and high homology to the tomato CF-2 gene for resistance to Cladosporium fulvus.

The rhg1 and Rfs2 sequences disclosed herein were used to isolate rhg1 and Rfs2 cDNAs according to methods well-known in the art. A representative rhg1 and Rfs2 cDNA is set forth as SEQ ID NO:1. This segment of the rhg1 and Rfs2 gene shows homology to the leucine-rich regions of the Arabidopsis hypothetical protein T18N14.120 (Gen Bank T46070) and tomato CF-2 resistance genes.

The rhg1 and Rfs2 sequences disclosed herein were used to isolate the gene encoding an RLK at the rhg1 and Rfs2 locus according to methods well-known in the art. A functional rhg1 and Rfs2 gene is set forth as SEQ ID NO: 3. This segment of the rhg1 and Rfs2 gene shows homology to the leucine-rich regions of the Arabidopsis hypothetical protein T18N14.120 (Gen Bank T46070) and tomato CF-2 resistance genes.

Genes underlying quantitative traits, or genes with related function, such as disease resistance, are often organized in clusters within the genome (e.g., Staskawicz (1995) Science 268:661-667). In the case of FRR/CN/SDS resistance, previous studies by the co-inventors of the present invention have suggested that the resistance trait in Forrest may be caused by four genes in a cluster with two pairs in close linkage or by a two-gene cluster with each gene displaying pleiotropy (Meksem et al., 1999). Thus, genomic DNA isolated and disclosed herein comprise multiple resistance gene sequences. Additional sequences derived from the FRR/CN/SDS resistance locus are set forth as SEQ ID NOs: 4 and 32. BLASTX analysis of these sequences reveals further homology to known proteins in other organisms, supporting that they comprise new gene sequences (Table 1). Of particular interest, BLASTX analysis of the sequences set forth as SEQ ID NOs: 1, 2, 4 and 32 reveals that the disclosed sequences have high homology to the T46070 GenBank entry described as hypothetical protein T18N 14.120 from Arabidopsis thaliana, high homology to the tomato CF-2 disease resistance genes encoding leucine-rich repeat proteins, and to the tomato CF-9 gene for resistance to Cladosporium fulvus (Table 1).

The present invention also pertains to resistance genes related to rhg1 and Rfs2. Sequences of additional putative FRR/CN/SDS resistance genes, set forth as SEQ ID NO: 32 were identified based on hybridization to rhg1 and Rfs2 sequences, as further described in Examples. BLASTX analysis of these sequences reveals further homology to known proteins in other organisms, supporting that they comprise new partial gene sequences. Of particular interest, BLASTX analysis of the sequences set forth as SEQ ID NOs: 32 reveals that several of the disclosed sequences have high homology to the T46070 GenBank entry described as hypothetical protein T18N14.120 from Arabidopsis thaliana, high homology to the tomato CF-2 disease resistance genes encoding leucine-rich repeat proteins, and to the tomato CF-9 gene for resistance to Cladosporium fulvus. Based on their hybridization to rhg1 and Rfs2 sequences, genes comprising any of SEQ ID NOs: 3 and 32 may also confer partial resistance to race 3 Heterodera glycines. It will be apparent to one having ordinary skill in the art that the disclosed sequences, or portion thereof, can be used to identify, confirm and/or screen for SDS, SCN and/or other resistance or for loci that confer SDS, SCN and/or other resistance.

### III.D. FRR/CN/SDS Resistance Gene Promoters

The term “promoter region” defines a nucleotide sequence within a gene that is positioned 5′ to a coding sequence of a same gene and functions to direct transcription of the coding sequence. The promoter region includes a transcriptional start site and at least one cis-regulatory element. The present invention encompasses nucleic acid sequences that comprise a promoter region of an FRR/CN/SDS resistance gene, or functional portion thereof.

The terms “cis-acting regulatory sequence” or “cis-regulatory motif” or “response element”, as used herein, each refer to a nucleotide sequence that enables responsiveness to a regulatory transcription factor. Responsiveness can encompass a decrease or an increase in transcriptional output and is mediated by binding of the transcription factor to the DNA molecule comprising the response element.

The term “transcription factor” generally refers to a protein that modulates gene expression by interaction with the cis-regulatory element and cellular components for transcription, including RNA Polymerase, Transcription Associated Factors (TAFs), chromatin-remodeling proteins, and any other relevant protein that impacts gene transcription.

The term “gene expression” generally refers to the cellular processes by which a biologically active polypeptide is produced from a DNA sequence.

A “functional portion” of a promoter gene fragment is a nucleotide sequence within a promoter region that is required for normal gene transcription. To determine nucleotide sequences that are functional, the expression of a reporter gene is assayed when variably placed under the direction of a promoter region fragment.

Promoter region fragments can be conveniently made by enzymatic digestion of a larger fragment using restriction endonucleases or DNAse I. Preferably, a functional promoter region fragment comprises less than the 6,500 bp upstream of Rhg1 Rfs2 (SEQ NO. 4) more preferably about 5,000 nucleotides, More preferable the 3,500 bp encoded on pSBHB96, more preferably 2,000 nucleotides, more preferably about 1,000 nucleotides, more preferably a functional promoter region fragment comprises about 500 nucleotides, even more preferably a functional promoter region fragment comprises about 100 nucleotides, and even more preferably a functional promoter region fragment comprises about 20 nucleotides.

Within a candidate promoter region or response element, the presence of regulatory proteins bound to a nucleic acid sequence can be detected using a variety of methods well known to those skilled in the art (Ausubel et al., 1992). Briefly, in vivo footprinting assays demonstrate protection of DNA sequences from chemical and enzymatic modification within living or permeabilized cells. Similarly, in vitro footprinting assays show protection of DNA sequences from chemical or enzymatic modification using protein extracts. Nitrocellulose filter-binding assays and gel electrophoresis mobility shift assays (EMSAs) track the presence of radio-labeled regulatory DNA elements based on provision of candidate transcription factors.

The terms “reporter gene” or “marker gene” or “selectable marker” each refer to a heterologous gene encoding a product that is readily observed and/or quantitated. A reporter gene is heterologous in that it originates from a source foreign to an intended host cell or, if from the same source, is modified from its original form. Non-limiting examples of detectable reporter genes that can be operably linked to a transcriptional regulatory region can be found in brown and PCT International Publication No. WO 97/47763. Preferred reporter genes for transcriptional analyses include the lacZ gene (See, e.g., Rose & Botstein (1983) Meth Enzymol 101:167-180), Green Fluorescent Protein (GFP) (Cubitt et al. (1995) Trends Biochem Sci 20:448-455), luciferase, or chloramphenicol acetyl transferase (CAT). Preferred reporter genes for stable transformation include but are not limited to antibiotic resistance genes. Any suitable reporter and detection method can be used, and it will be appreciated by one of skill in the art that no particular choice is essential to or a limitation of the present invention.

An amount of reporter gene can be assayed by any method for qualitatively or preferably, quantitatively determining presence or activity of the reporter gene product. The amount of reporter gene expression directed by each test promoter region fragment is compared to an amount of reporter gene expression to a control construct comprising the reporter gene in the absence of a promoter region fragment. A promoter region fragment is identified as having promoter activity when there is significant increase in an amount of reporter gene expression in a test construct as compared to a control construct. The term “significant increase”, as used herein, refers to an quantified change in a measurable quality that is larger than the margin of error inherent in the measurement technique, preferably an increase by about 2-fold or greater relative to a control measurement, more preferably an increase by about 5-fold or greater, and most preferably an increase by about 10-fold or greater.

A representative FRR/CN/SDS resistance gene promoter, the rhg1 and Rfs2 promoter, is set forth as SEQ ID NO: 2 and 4. The rhg1 and Rfs2 promoter is useful for directing gene expression of heterologous sequences in vivo or in assays to identify modulators of rhg1 and Rfs2 expression, described further herein below.

The present invention further provides an isolated FRR/CN/SDS resistance gene promoter region, or functional portion thereof, comprising an about 82.157 kb fragment of soybean genomic clone 73P6 between BamHI restriction sites and 21D9 between HinDIII restriction site. The genomic clone is available from the Forrest BAC library described in Meksem et al (2000), Theor Appl Genet. 101 5/6: 747-755, available through Southern Illinois University-Carbondale (Carbondale, Ill.), Texas A&M University BAC center (College Station, Tex.), and Research Genetics (Huntsville, Ala.). An isolated FRR/CN/SDS resistance gene promoter region, or functional portion thereof, comprising a 9.772 kb fragment of soybean genomic clone 21d9A2 is also disclosed as SEQ NO 2.

### III.E. Chimeric Genes

The present invention also encompasses chimeric genes comprising the disclosed FRR/CN/SDS resistance gene sequences. The term “chimeric gene”, as used herein, refers to an FRR/CN/SDS resistance gene promoter region operably linked to an open reading frame, wherein the nucleotide sequence created is not naturally occurring. In this regard, the open reading frame is also described as a “heterologous sequence”. The term “chimeric gene” also encompasses a promoter region operably linked to an FRR/CN/SDS resistance gene coding sequence, a nucleotide sequence producing an antisense RNA molecule, a RNA molecule having tertiary structure, such as a hairpin structure, or a double-stranded RNA molecule.

The term “operably linked”, as used herein, refers to a promoter region that is connected to a nucleotide sequence in such a way that the transcription of that nucleotide sequence is controlled and regulated by that promoter region. Techniques for operatively linking a promoter region to a nucleotide sequence are well known in the art.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native cis-regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

### IV. Polypeptide Sequences of FRR/CN/SDS Resistance Proteins

The polypeptides provided by the present invention include the isolated polypeptide of SEQ ID NO: 3, fusion proteins comprising FRR/CN/SDS resistance gene amino acid sequences, biologically functional analogs, and polypeptides that cross-react with an antibody that specifically recognizes an FRR/CN/SDS resistance gene polypeptide like SEQ NO 31.

The term “isolated”, as used in the context of a polypeptide, indicates that the polypeptide exists apart from its native environment and is not a product of nature. An isolated polypeptide can exist in a purified form or can exist in a non-native environment such as, for example, in a transgenic host cell.

The term “purified”, when applied to a polypeptide, denotes that the polypeptide is essentially free of other cellular components with which it is associated in the natural state. Preferably, a polypeptide is a homogeneous solid or aqueous solution. Purity and homogeneity are typically determined using analytical chemistry techniques such as polyacrylamide gel electrophoresis or high performance liquid chromatography. A polypeptide that is the predominant species present in a preparation is substantially purified. The term “purified” denotes that a polypeptide gives rise to essentially one band in an electrophoretic gel. Particularly, it means that the polypeptide is at least about 50% pure, more preferably at least about 85% pure, and most preferably at least about 99% pure.

The term “substantially identical” in the context of two or more polypeptides sequences is measured by (a) polypeptide sequences having about 35%, or 45%, or preferably from 45-55%, or more preferably 55-65%, or most preferably 65% or greater amino acids that are identical or functionally equivalent. Percent “identity” and methods for determining identity are defined herein under the heading Nucleotide and Amino Acid Sequence Comparisons.

Substantially identical polypeptides also encompass two or more polypeptides sharing a conserved three-dimensional structure. Computational methods can be used to compare structural representations, and structural superpositions can be generated and easily tuned to identify similarities around important active sites or ligand binding sites. See Henikoff et al. (2000) Electrophoresis 21(9):1700-1706; Huang et al. (2000) Pac Symp Biocomput 230-241; Saqi et al., 1999; and Barton (1998) Acta Crystallogr D Biol Crystallogr 54:1139-1146.

The term “functionally equivalent” in the context of amino acid sequences is well known in the art and is based on the relative similarity of the amino acid side-chain substituents. See Henikoff and Henikoff (2000) Adv Protein Chem 54:73-97. Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al. (1982) J Mol Biol 157:105.). It is known that certain amino acids can be substituted for other amino acids having a similar hydropathic index or score and still retain a similar biological activity. In making changes based upon the hydropathic index, the substitution of amino acids whose hydropathic indices are within ±0.2 of the original value is preferred, those which are within ±0.1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101 states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±0.1); glutamate (+3.0±0.1); serine (+0.3); asparagine (+0.2); glutamine (+0.2); glycine (0); threonine (−0.4); proline (−0.5±0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±0.2 of the original value is preferred, those which are within ±0.1 of the original value are particularly preferred, and those within ±0.5 of the original value are even more particularly preferred.

The present invention also encompasses FRR/CN/SDS resistance gene polypeptide fragments or functional portions of an FRR/CN/SDS resistance gene polypeptide. Such functional portion need not comprise all or substantially all of the amino acid sequence of a native resistance gene product. The term “functional” includes any biological activity or feature of FRR/CN/SDS resistance gene, including immunogenicity.

The present invention also includes longer sequences comprising an FRR/CN/SDS resistance gene polypeptide, or portion thereof. For example, one or more amino acids can be added to the N-terminal or C-terminal of an FRR/CN/SDS resistance gene polypeptide. Fusion proteins comprising FRR/CN/SDS resistance gene polypeptide sequences are also provided within the scope of the present invention. Methods of preparing such proteins are known in the art.

The present invention also encompasses functional analogs of an FRR/CN/SDS resistance gene polypeptide. Functional analogs share at least one biological function with an FRR/CN/SDS resistance gene polypeptide. An exemplary function is immunogenicity. In the context of amino acid sequence, biologically functional analogs, as used herein, are peptides in which certain, but not most or all, of the amino acids can be substituted. Functional analogs can be created at the level of the corresponding nucleic acid molecule, altering such sequence to encode desired amino acid changes. In one embodiment, changes can be introduced to improve the antigenicity of the protein. In another embodiment, an FRR/CN/SDS resistance gene polypeptide sequence is varied so as to assess the activity of a mutant FRR/CN/SDS resistance gene polypeptide. In still another embodiment, amino acid changes can be made to improve the stability of the polypeptide.

Isolated polypeptides and recombinantly produced polypeptides can be purified and characterized using a variety of standard techniques that are well known to the skilled artisan. See, e.g. Ausubel et al. (1992); Bodanszky et al., 1976; and Zimmer et al. (1993) Peptides, pp. 393B394, ESCOM Science Publishers, B. V., Afzal and Lightfoot (2009) Protein Expr Purif 53: 346-355.

### V. Nucleotide and Amino Acid Sequence Comparisons

The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain at least some of biological activity of the natural gene, gene product, or sequence. Such sequences include “mutant” sequences, or sequences wherein the biological activity is altered to some degree but retains at least some of the original biological activity. The term “naturally occurring”, as used herein, is used to describe a composition that can be found in nature as distinct from being artificially produced by man. For example, a protein or nucleotide sequence present in an organism, which can be isolated from a source in nature and which has not been intentionally modified by man in the laboratory, is naturally occurring.

For sequence comparison, typically one sequence is regarded as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman (1981) Adv Appl Math 2:482, by the homology alignment algorithm of Needleman & Wunsch (1970) J Mol Biol 48:443, by the search for similarity method of Pearson & Lipman (1988) Proc Natl Acad Sci USA 85:2444, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel et al. (1992).

A preferred algorithm for determining percent sequence identity and sequence similarity is the BLAST algorithm, which is described in Altschul et al. (1990) J Mol Biol 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff and Henikoff (1989) Proc Natl Acad Sci USA 89:10915.

In addition to calculating percent sequence identity, the BLAST algorithm also performs a statistical analysis of the similarity between two sequences. See, e.g., Karlin and Altschul (1993) Proc Natl Acad Sci USA 90:5873-5887. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

VI. Method for Detecting a Nucleic Acid Molecule Associated with FRR/CN/SDS Resistance

In another aspect of the invention, a method is provided for detecting a nucleic acid molecule that encodes an FRR/CN/SDS resistance polypeptide. Such methods can be used to detect FRR/CN/SDS resistance gene variants and related resistance gene sequences. The disclosed methods facilitate genotyping, cloning, gene mapping, and gene expression studies.

### VI.A. Genetic Variants

In one embodiment, genetic assays based on nucleic acid molecules of the present invention can be used to screen for genetic variants by a number of PCR-based techniques, including single-strand conformation polymorphism (SSCP) analysis (Orita et al. (1989) Proc Natl Acad Sci USA 86(8):2766-2770), SSCP/heteroduplex analysis, enzyme mismatch cleavage, direct sequence analysis of amplified exons (Kestila et al. (1998) Mol Cell 1 (4):575-582; Yuan et al. (1999) Hum Mutat 14(5):440-446), allele-specific hybridization (Stoneking et al. (1991) Am J Hum Genet 48(2):370-82), and restriction analysis of amplified genomic DNA containing the specific mutation. Automated methods can also be applied to large-scale characterization of single nucleotide polymorphisms (Brookes (1999) Gene 234(2):177-186; Wang et al. (1998) Science 280(5366):1077-82). Preferred detection methods are non-electrophoretic, including, for example, the TaqMan™ allelic discrimination assay, PCR-OLA, molecular beacons, padlock probes, and well fluorescence. See Landegren et al. (1998) Genome Res 8:769-776.

In a preferred embodiment, genetic markers for FRR/CN/SDS resistance disclosed herein are used in a PCR-based genotyping assay, preferably, a TaqMan™ assay as disclosed in Example 6. The TaqMan™ allelic discrimination assay is based on the 5′ nuclease activity of Taq polymerase and detection of a fluorescent reporter during or after PCR reactions (Livak et al. (1995) PCR Meth and Applic 4:357-362; Livak et al. (1995) Nat Genet. 9:341-342). Each TaqMan™ probe consists of a 25-35 base oligonucleotide complementary to one of two alleles with a 3′ quencher dye attached (6-carboxy-N, N,N′5N′ tetrachlorofluorescein; TAMRA). The oligomer complimentary to allele 1 is linked covalently to a 5′ reporter dye (6-carboxy-4,7,2′,7′, tetrachlorofluorescenin; TET) while allele 2 is linked to a dye that fluoresces at a distinct wavelength (6-carboxyfluorescein; FAM). PCR directed by flanking oligomers of 18-20 bases causes degradation during the extension phase of the oligomer that hybridizes most efficiently to the polymorphic site(s) in the sample. Adaptations can make the assay chemistry suitable for multiplexing (Nasarabadi et al. (1999) Bio Techniques 27:1116-1117) and miniaturization (Kalinina et al. (1997) Nucl Acids Res 25:1999-2004) to reduce cost and increase throughput.

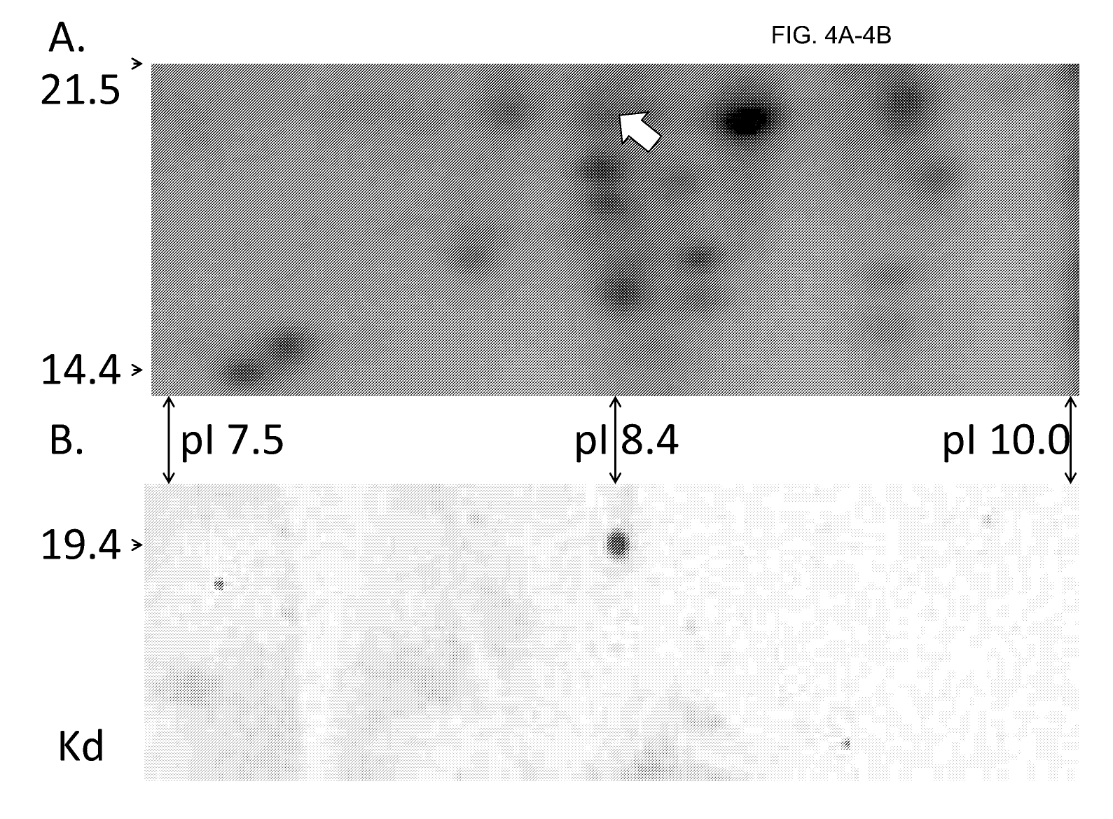

The present invention discloses sequences suitable for use with the TaqMan™ method for genotyping FRR/CN/SDS resistance, further disclosed in Example 6. As one example, the TaqMan™ assay was used to distinguish between three polymorphisms in alleles of the Rhg1 and Rfs2 gene (FIG. 4). Genomic DNA samples were analyzed using the TaqMan™ PCR protocol (Livak et al., 1995a, 1995b). Using the raw fluorescence signals of the reporter dyes FAM and TET from the “dye component” field of the sequence detection software, two grouping methods were performed. Each method detected four distinct populations (FIG. 4). The four populations could be assigned according to the FAM:TET ratio based on where the heterogeneous class cut-off was placed.

For the TaqMan™ selection, two grouping methods were arbitrarily selected to attempt to accurately separate heterogeneous lines from homogeneous lines at each allele. For grouping method 1 (Taqman™ 1) a stringent cut-off was used to reduce the number called as potentially heterogeneous. Fluorophore ratios were as follows; no amplification (FAM and TET both less than 6 units); allele 1 homozygous (FAM less than 7, TET greater than 7); allele 2 homozygous (FAM greater than 10, TET less than 5); and heterogeneous for allele 1 and allele 2 (FAM greater than 7, TET 5-8). For TaqMan™ selection grouping method 2 (TaqMan™ 2), a lower stringency cut-off value was used to increase the number called as potentially heterogeneous. Ratios were: no amplification (FAM and TET both less than 6 units); allele 1 homozygous (FAM less than 5, TET greater than 7); allele 2 homozygous (FAM greater than 10, TET less than 5); and heterogeneous for allele 1 and allele 2 (FAM greater than 5, TET 5-9).

Based on the Fl of the ExF RIL population, the 86 selected individuals were classified into 3 classes: 15 resistant, 60 susceptible and 11 segregating lines. TaqMan™ analysis of 86 individuals from the RILs by method 1 (high stringency) shows a strong agreement between allele 1 and susceptibility to SCN (60 from the 60 susceptible lines were allele 1 type). However, there was lesser agreement between allele 2 and resistance to SCN (only 15 lines from the 23 lines showing the presence of allele 2 were resistant by phenotype) due to the segregation of Rhg4, the second gene necessary for resistance to SCN in Forrest. Of the 11 lines known to be heterogeneous for the resistance to SCN phenotype, five should segregate at Rhg4 and six at rhg1 Rfs2. TaqMan™ method 1 identified one among the five classified as heterogenous (the 5 include 4 miss-classified lines, see below). TaqMan™ method 2 identified all five among the 11 classified as heterogenous, however the 11 include 6 miss-classified lines.

The genotype and phenotype were generally in close agreement among the eighty six genomic DNA samples analyzed using the TaqMan™ PCR protocol. The lesser agreement between Allele 2 and resistance to SCN (15 of 23) was shown to be due to the segregation of Rhg4, by scoring of the BARC-Satt 309 marker (Meksem et al., 1999). The bias toward a higher frequency of allele 1 is caused by sampling error (Chang et al., 1997). The accuracy of genotyping was high by the TaqMan™ assay and was better than one pass gel electrophoresis (Prabhu et al., 1999). Even compared to a highly optimized gel electrophoresis assay reported herein the assays were not significantly different in accuracy for detecting the genotypes within the F₅ derived RILs in a single pass assay. Exactly 78 of the 86 tested with both, TaqMan™ and gel electrophoresis results agreed. There were 5 errors with Taqman™ (94% accurate) and 3 errors with gel electrophoresis (96% accurate) judged by replicated genotyping (not shown) and the phenotype. Low frequencies of error are important to the accurate selection of resistance (Cregan et al., 1999a; Prabhu et al., 1999) and in the generation of accurate genetic maps (Cregan et al., 1999b).

### VI.B. Cloning of FRR/CN/SDS Resistance Genes and Related Genes

The nucleic acids of the present invention can be used to clone genes and genomic DNA comprising the sequences. Alternatively, the nucleic acids of the present invention can be used to clone genes and genomic DNA of related sequences. For this purpose, representative probes, hybridization conditions, and PCR primers are described in the section entitled Nucleotide Sequences of FRR/CN/SDS Resistance Genes and Associated Markers herein above and in Examples 4 and 5. Preferably, the nucleic acids used for this method comprise sequences set forth as any one of SEQ ID NOs: 1, 2 and 4, more preferably SEQ ID NOs: 2.

In another embodiment, the present invention provides a method of positional cloning of genes and other sequences located adjacent or near the disclosed sequences within the soybean genome. The method comprises: (a) identifying a first nucleic acid genetically linked to a FRR/CN/SDS resistance locus; and (b) cloning the first nucleic acid. Optionally, the first nucleic acid can comprise the rhg1 and Rfs2 and SDS locus or the Rhg4 locus. Preferably, the FRR/CN/SDS resistance locus corresponds to a nucleic acid selected from any one of SEQ ID NOs: 1, 2, 4 and 32.

Positional cloning first involves creating a physical map of a contig (contiguous overlapping of cloned DNA inserts), in the genomic region encompassing one or more marker loci and the target gene. The target gene is then identified and isolated within one or more clones residing in the contig. The cloned gene can be used according to any suitable method known in the art, including, for example, genetic tudies, transformation, and the development of novel phenotypes.

Mapped SCN, SDS, or SCN and SDS markers, especially those most closely linked to FRR/CN/SDS resistance can be used to identify homologous clones from soybean genomic libraries, including, for example, soybean genomic libraries made in bacterial artificial chromosomes (BAC), yeast artificial chromosomes (YAC), or P1 bacteriophage. These types of vectors are preferred for positional cloning because they have the capacity to carry larger DNA inserts than possible with other vector technologies. These larger DNA inserts allow the researcher to move physically farther along the chromosome by identifying overlapping clones. Exemplary libraries available for positional cloning efforts in soybean include those described by Meksem et al., 2000; Kanazin et al. (1996) Proc Natl Acad Sci USA 93(21):11746-11750; Zhu et al. (1996) Mol Gen Genet. 252:483-488. Exemplary hybridization methods are disclosed in Examples 4 and 5.

Mapped SCN, SDS, or SCN and SDS markers can be used as DNA probes to hybridize and select homologous genomic clones from such libraries. Alternatively, the DNA of mapped marker clones are sequenced to design PCR primers that amplify and therefore identify homologous genomic clones from such libraries. Either method is used to identify large-insert soybean clones that is then used to start or finish a contig constructed in chromosome walking to clone an SCN, SDS, or SCN and SDS resistance QTL.

As examples, the positional cloning strategy was successfully used to clone the cystic fibrosis gene in humans (Rommens et al. (1989) Science 245:1059-1065), an omega-3 desaturase gene in Arabidopsis Arondel et al. (1992) Science 258:1353-1355), a protein kinase gene (Pto) conferring fungal resistance in tomato (Martin et al. (1993) Science 262:1432-1436), a YAC clone containing the jointless gene that suppresses abscission of flowers and fruit in tomato (Zhang et al. (1994) Mol Gen Genet. 244:613-621), and sequences comprising the rhg1 and Rfs2 genes, disclosed herein.

### VI.C. Mapping Methods

The isolated and purified polynucleotide sequences disclosed herein can also be used in a variety of applications pertaining to mapping SCN and SDS resistance. For example, the isolated polynucleotides disclosed herein are useful in studies of genome organization; in gene structure and organization experiments; in BAC-FISH experiments; in chromosome painting techniques; and in chromosome manipulation.

Thus, in accordance with the present invention, the nucleic acid sequences which encode FRR/CN/SDS resistance polypeptides can also be used to generate hybridization probes which are useful for mapping naturally occurring genomic sequences and/or resistance loci. The sequences can be mapped to a particular chromosome or to a specific region of the chromosome using well-known techniques. Such techniques include FISH, FACS, or artificial chromosome constructions, such as yeast artificial chromosomes, bacterial artificial chromosomes, bacterial P1 constructions or single chromosome cDNA libraries as reviewed in Price (1993) Blood Rev 7:127-134, and Trask (1991) Trends Genet. 7:149-154.

FISH (as described in Verma et al. (1988) Human Chromosomes: A Manual of Basic Techniques, Pergamon Press, New York, N.Y.) can be correlated with other physical chromosome mapping techniques and genetic map data. Examples of genetic map data can be found in the 1994 Genome Issue of Science (265:1981f). Correlation between the location of the gene encoding SCN, SDS, or both SCN and SDS resistance on a physical chromosomal map and another resistance characteristic, or lack thereof, can help delimit the region of DNA associated with that genetic characteristic. The nucleotide sequences of the subject invention can be used to detect differences in gene sequences between normal, carrier, or susceptible individuals.

In situ hybridization of chromosomal preparations and physical mapping techniques such as linkage analysis and chromosomal painting using established chromosomal markers can be used for extending genetic maps. Often the placement of a gene on the chromosome of another plant species, such as tomato species or other soybean species, reveals associated markers also found in other plants such as soybeans even if the number or arm of a particular chromosome is not known. New sequences can be assigned to chromosomal arms, or parts thereof, by physical mapping. This provides valuable information to investigators searching for resistance or other genes using positional cloning or other gene discovery techniques. Once the resistance or other gene has been crudely localized by genetic linkage to a particular genomic region, any sequences mapping to that area can represent associated or regulatory genes for further investigation. The nucleotide sequences of the present invention can thus also be used to detect differences in the chromosomal location due to translocation, inversion, etc. among normal, carrier, or susceptible individuals, and to detect gene regulatory sequences (e.g. promoters).

Hybridization of the subject DNAs to reference chromosomes can also be performed to give information on relative copy numbers of sequences. Normalization is required to obtain absolute copy number information. One convenient method to do this is to hybridize a probe, for example a cosmid specific to some single locus in the normal haploid genome, to the interphase nuclei of the subject cell or cell population(s) (or those of an equivalent cell or representative cells therefrom, respectively). Quantification of the hybridization signals in a representative population of such nuclei gives the absolute sequence copy number at that location. Given that information at one locus, the intensity (ratio) information from the hybridization of the subject DNA(s) to the reference condensed chromosomes gives the absolute copy number over the rest of the genome. In practice, use of more than one reference locus can be desirable. In this case, the best fit of the intensity (ratio) data through the reference loci can give a more accurate determination of absolute sequence copy number over the rest of the genome.

Thus, the methods of the present invention can provide information on the absolute copy numbers of substantially all RNA or DNA sequences in subject cell(s) or cell population(s) as a function of the location of those sequences in a reference genome. Additionally, chromosome painting probes can be prepared using the markers and sequence data herein disclosed. Hybridization with one or more of such probes indicates the absolute copy numbers of the sequences to which the probes bind.

Further, when the subject nucleic acid sequences are DNA, the reference copy numbers can be determined by Southern analysis. When the subject nucleic acid sequences are RNA, the reference copy numbers can be determined by Northern analysis.

### VI.D. Assays Kits

In another aspect, the present invention provides assay kits for detecting the presence, in biological samples, of a polynucleotide that encodes a polypeptide of the present invention or of a chromosome bearing a gene or locus of the present invention, the kits comprising a first container that contains a second polynucleotide identical or complementary to a segment of at least 10 contiguous nucleotide bases of, as a preferred example, any of SEQ ID NOs: 1, 2, 4 and 32.

### VII. Recombinant Expression B Expression Cassettes

The term “expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest can be chimeric. The expression cassette can also be one which is naturally occurring but has been obtained in a recombinant form useful for heterologous expression. The expression cassettes can also comprise any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments.

### VII.A. Promoters

The expression of the nucleotide sequence in the expression cassette can be under the control of a constitutive promoter or an inducible promoter which initiates transcription only when the host cell is exposed to some particular external stimulus. For bacterial production of a FRR/CN/SDS resistance polypeptide, exemplary promoters include Simian virus 40 early promoter, a long terminal repeat promoter from retrovirus, an actin promoter, a heat shock promoter, and a metallothionein protein. For in vivo production of a FRR/CN/SDS resistance polypeptide in plants, exemplary constituitve promoters are derived from the CaMV ³⁵S, rice actin, and maize ubiquitin genes, each described herein below. Exemplary inducible promoters for this purpose include the chemically inducible PR-1a promoter and a wound-inducible promoter, also described herein below.

Selected promoters can direct expression in specific cell types (such as leaf epidermal cells, mesophyll cells, root cortex cells) or in specific tissues or organs (roots, leaves or flowers, for example). Exemplary tissue-specific promoters include well-characterized root-, pith-, and leaf-specific promoters, each described herein below.

Depending upon the host cell system utilized, any one of a number of suitable promoters can be used. Promoter selection can be based on expression profile and expression level. The following are non-limiting examples of promoters that can be used in the expression cassettes.

### VII.A.1. Constitutive Expression

35S Promoter. The CaMV 35S promoter can be used to drive constitutive gene expression. Construction of the plasmid pCGN1761 is described in the published patent application EP 0 392 225, which is hereby incorporated by reference. pCGN1761 contains the “double” CaMV 35S promoter and the tml transcriptional terminator with a unique EcoRI site between the promoter and the terminator and has a pUC-type backbone. A derivative of pCGN1761 is constructed which has a modified polylinker which includes NotI and XhoI sites in addition to the existing EcoRI site. This derivative is designated pCGN1761ENX. pCGN1761ENX is useful for the cloning of cDNA sequences or gene sequences (including microbial ORF sequences) within its polylinker for the purpose of their expression under the control of the 35S promoter in transgenic plants. The entire 35S promoter-gene sequence-tmI terminator cassette of such a construction can be excised by HindIII, SphI, SalI, and XbaI sites 5′ to the promoter and XbaI, BamHI and BgII sites 3′ to the terminator for transfer to transformation vectors such as those described below. Furthermore, the double 35S promoter fragment can be removed by 5′ excision with HindIII, SphI, SalI, XbaI, or PstI, and 3′ excision with any of the polylinker restriction sites (EcoRI, NotI or XhoI) for replacement with another promoter.

Actin Promoter. Several isoforms of actin are known to be expressed in most cell types and consequently the actin promoter is a good choice for a constitutive promoter. In particular, the promoter from the rice ActI gene has been cloned and characterized (McElroy et al. (1990) Plant Cell 2:163-171). A 1.3 kb fragment of the promoter was found to contain all the regulatory elements required for expression in rice protoplasts. Furthermore, numerous expression vectors based on the ActI promoter have been constructed specifically for use in monocotyledons (McElroy et al. (1991) Mol Gen Genet. 231:150-160). These incorporate the ActI-intron 1, AdhI 5′ flanking sequence and AdhI-intron I (from the maize alcohol dehydrogenase gene) and sequence from the CaMV 35S promoter. Vectors showing highest expression were fusions of 35S and ActI intron or the ActI 5′ flanking sequence and the ActI intron. Optimization of sequences around the initiating ATG (of the GUS reporter gene) also enhanced expression. The promoter expression cassettes described by McElroy et al. (1991) can be easily modified for gene expression and are particularly suitable for use in monocotyledonous hosts. For example, promoter-containing fragments is removed from the McElroy constructions and used to replace the double 35S promoter in pCGN1761ENX, which is then available for the insertion of specific gene sequences. The fusion genes thus constructed can then be transferred to appropriate transformation vectors. In a separate report, the rice ActI promoter with its first intron has also been found to direct high expression in cultured barley cells (Chibbar et al. (1993) Plant Cell Rep 12:506-509).

Ubiquitin Promoter. Ubiquitin is another gene product known to accumulate in many cell types and its promoter has been cloned from several species for use in transgenic plants (e.g. sunflower—Binet et al. (1991) Plant Science 79: 87-94 and maize—Christensen et al. (1989) Plant Molec Biol 12:619-632). The maize ubiquitin promoter has been developed in transgenic monocot systems and its sequence and vectors constructed for monocot transformation are disclosed in the patent publication EP 0 342 926 which is herein incorporated by reference. Taylor et al. (1993) Plant Cell Rep 12:491-495 describe a vector (pAHC25) that comprises the maize ubiquitin promoter and first intron and its high activity in cell suspensions of numerous monocotyledons when introduced via microprojectile bombardment. The ubiquitin promoter is suitable for gene expression in transgenic plants, especially monocotyledons. Suitable vectors are derivatives of pAHC25 or any of the transformation vectors described in this application, modified by the introduction of the appropriate ubiquitin promoter and/or intron sequences.

### VII.A.2. Inducible Expression

Chemically Inducible PR-1a Promoter. The double 35S promoter in pCGN1761ENX can be replaced with any other promoter of choice which will result in suitably high expression levels. By way of example, one of the chemically regulatable promoters described in U.S. Pat. No. 5,614,395 can replace the double 35S promoter. The promoter of choice is preferably excised from its source by restriction enzymes, but can alternatively be PCR-amplified using primers that carry appropriate terminal restriction sites. Should PCR-amplification be undertaken, then the promoter should be re-sequenced to check for amplification errors after the cloning of the amplified promoter in the target vector. The chemical/pathogen regulated tobacco PR-1a promoter is cleaved from plasmid pCIB1004 (for construction, see EP 0 332 104, which is hereby incorporated by reference) and transferred to plasmid pCGN1761 ENX (Uknes et al. (1992) The Plant Cell 4:645-656).

pCIB 1004 is cleaved with NcoI and the resultant 3′ overhang of the linearized fragment is rendered blunt by treatment with T4 DNA polymerase. The fragment is then cleaved with HindIII and the resultant PR-1a promoter-containing fragment is gel purified and cloned into pCGN1761ENX from which the double 35S promoter has been removed. This is done by cleavage with XhoI and blunting with T4 polymerase, followed by cleavage with HindIII and isolation of the larger vector-terminator containing fragment into which the pCIB 1004 promoter fragment is cloned. This generates a pCGN1761 ENX derivative with the PR-1a promoter and the tml terminator and an intervening polylinker with unique EcoRI and NotI sites. The selected coding sequence can be inserted into this vector, and the fusion products (i.e. promoter-gene-terminator) can subsequently be transferred to any selected transformation vector, including those described below. Various chemical regulators can be employed to induce expression of the selected coding sequence in the plants transformed according to the present invention, including the benzothiadiazole, isonicotinic acid, and salicylic acid compounds disclosed in U.S. Pat. Nos. 5,523,311 and 5,614,395, herein incorporated by reference.

Wound-Inducible Promoters. Wound-inducible promoters can also be suitable for gene expression. Numerous such promoters have been described (e.g. Xu et al. (1993) Plant Molec Biol 22:573-588; Logemann et al. (1989) Plant Cell 1:151-158; Rohrmeier & Lehle (1993) Plant Molec Biol 22:783-792; Firek et al. (1993) Plant Molec Biol 22:129-142; Warner et al. (1993) Plant J 3:191-201) and all are suitable for use with the instant invention. Logemann et al. (1989) describe the 5′ upstream sequences of the dicotyledonous potato wunI gene. Xu et al. (1993) show that a wound-inducible promoter from the dicotyledon potato (pin2) is active in the monocotyledon rice. Further, Rohrmeier & Lehle (1993) describe the cloning of the maize Wipl cDNA which is wound induced and which can be used to isolate the cognate promoter using standard techniques. Similarly, Firek et al. (1993) and Warner et al. (1993) have described a wound-induced gene from the monocotyledon Asparagus officinalis, which is expressed at local wound and pathogen invasion sites. Using cloning techniques well known in the art, these promoters can be transferred to suitable vectors, fused to the genes pertaining to this invention, and used to express these genes at the sites of plant wounding.

### VII.A.3. Tissue-Specific Expression

Root Promoter. Another pattern of gene expression is root expression. A suitable root promoter is described by de Framond (1991) FEBS 290:103-106 and also in the published patent application EP 0 452 269, which is herein incorporated by reference. This promoter is transferred to a suitable vector such as pCGN1761ENX for the insertion of a selected gene and subsequent transfer of the entire promoter-gene-terminator cassette to a transformation vector of interest.

Pith Promoter. International Publication No. WO 93/07278, which is herein incorporated by reference, describes the isolation of the maize trpA gene, which is preferentially expressed in pith cells. The gene sequence and promoter extending up to −1726 bp from the start of transcription are presented. Using standard molecular biological techniques, this promoter, or parts thereof, can be transferred to a vector such as pCGN1761 where it can replace the 35S promoter and be used to drive the expression of a foreign gene in a pith-preferred manner. In fact, fragments containing the pith-preferred promoter or parts thereof can be transferred to any vector and modified for utility in transgenic plants.

Leaf Promoter. A maize gene encoding phosphoenol carboxylase (PEPC) has been described by Hudspeth & Grula (1989) Plant Molec Biol 12:579-589. Using standard molecular biological techniques the promoter for this gene can be used to drive the expression of any gene in a leaf-specific manner in transgenic plants.

### VII.B. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tml terminator, the nopaline synthase terminator and the pea rbcS E9 terminator. These can be used in both monocotyledons and dicotyledons.

### VII.C. Sequences for the Enhancement or Regulation of Expression

Numerous sequences have been found to enhance gene expression from within the transcriptional unit and these sequences can be used in conjunction with the genes of this invention to increase their expression in transgenic plants.

If desired, modifications around the cloning sites can be made by the introduction of sequences that can enhance translation. This is particularly useful when overexpression is desired. For example, pCGN1761 ENX can be modified by optimization of the translational initiation site as disclosed in U.S. Pat. No. 5,639,949, incorporated herein by reference.

Various intron sequences have been shown to enhance expression, particularly in monocotyledonous cells. For example, the introns of the maize AdhI gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al. (1987) Genes Develop 1:1 183-1200). In the same experimental system, the intron from the maize bronzel gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. (1987) Nucl Acids Res 15:8693-8711; Skuzeski et al. (1990) Plant Molec Biol 15:65-79).

### VII.D. Targeting of the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al. (1988) J Biol Chem 263:15104-15109). These signal sequences can be fused to heterologous gene products to effect the import of heterologous products into the chloroplast (van den Broeck et al. (1985) Nature 313:358-363). DNA encoding for appropriate signal sequences can be isolated from the 5′ end of the cDNAs encoding the RUBISCO protein, the CAB protein, the EPSP synthase enzyme, the GS2 protein and many other proteins which are known to be chloroplast localized. See also, U.S. Pat. No. 5,639,949, herein incorporated by reference.

Other gene products are localized to other organelles such as the mitochondrion and the peroxisome (e.g. Unger et al. (1989) Plant Molec Biol 13:411-418). The cDNAs encoding these products can also be manipulated to effect the targeting of heterologous gene products to these organelles. Examples of such sequences are the nuclear-encoded ATPases and specific aspartate amino transferase isoforms for mitochondria. Targeting cellular protein bodies has been described by Rogers et al. (1989) Proc Natl Acad Sci USA 82:6512-6516).

In addition, sequences have been characterized which cause the targeting of gene products to other cell compartments. Amino terminal sequences are responsible for targeting to the ER, the apoplast, and extracellular secretion from aleurone cells (Koehler & Ho (1990) Plant Cell 2:769-783). Additionally, amino terminal sequences in conjunction with carboxy terminal sequences are responsible for vacuolar targeting of gene products (Shinshi et al. (1990) Plant Molec Biol 14:357-368).

By the fusion of the appropriate targeting sequences described above to transgene sequences of interest, it is possible to direct the transgene product to any organelle or cell compartment. For chloroplast targeting, for example, the chloroplast signal sequence from the RUBISCO gene, the CAB gene, the EPSP synthase gene, or the GS2 gene is fused in frame to the amino terminal ATG of the transgene. The signal sequence selected should include the known cleavage site, and the fusion constructed should take into account any amino acids after the cleavage site which are required for cleavage. In some cases this requirement can be fulfilled by the addition of a small number of amino acids between the cleavage site and the transgene ATG or, alternatively, replacement of some amino acids within the transgene sequence. Fusions constructed for chloroplast import can be tested for efficacy of chloroplast uptake by in vitro translation of in vitro transcribed constructions followed by in vitro chloroplast uptake using techniques described by Bartlett et al. (1982) in Methods in Chloroplast Molecular Biology, Edelmann et al. (Eds.), pp 1081-1091, Elsevier and Wasmann et al. (1986) Mol Gen Genet. 205:446-453.

These construction techniques are well known in the art and are equally applicable to mitochondria and peroxisomes.

The above-described mechanisms for cellular targeting can be utilized not only in conjunction with their cognate promoters, but also in conjunction with heterologous promoters so as to effect a specific cell-targeting goal under the transcriptional regulation of a promoter that has an expression pattern different to that of the promoter from which the targeting signal derives.

### VIII. Recombinant Expression B Vectors

Suitable expression vectors which can be used include, but are not limited to, the following vectors or their derivatives: human or animal viruses such as vaccinia virus or adenovirus, yeast vectors, bacteriophage vectors (e.g., lambda phage), and plasmid and cosmid DNA vectors.

Numerous vectors available for plant transformation are known to those of ordinary skill in the plant transformation arts, and the genes pertinent to this invention can be used with any such vectors. Exemplary vectors include pCIB200, pCIB2001, pCIB10, pCIB3064, pSOG19, and pSOG35, each described herein below. The selection of vector will depend upon the preferred transformation technique and the target species for transformation.

### VIII.A. Agrobacterium Transformation Vectors.

Many vectors are available for transformation using Agrobacterium tumefaciens. These typically carry at least one T-DNA border sequence and include vectors such as pBIN19 (Bevan (1984) Nucl Acids Res 12:8711-8721) and pXYZ. Below, the construction of two typical vectors suitable for Agrobacterium transformation is described.

pCIB200 and pCIB2001. The binary vectors pcIB200 and pCIB2001 are used for the construction of recombinant vectors for use with Agrobacterium and are constructed in the following manner. pTJS75kan is created by Nan digestion of pTJS75 (Schmidhauser & Helinski (1985) J Bacteriol 164:446-455) allowing excision of the tetracycline-resistance gene, followed by insertion of an AccI fragment from pUC4K carrying an NPTII (Messing & Vierra (1982) Gene 19:259-268; Bevan et al. (1983) Nature 304:184-187; McBride et al. (1990) Plant Molecular Biology 14:266-276). XhoI linkers are ligated to the EcoRV fragment of PCIB7 which contains the left and right T-DNA borders, a plant selectable nos/nptll chimeric gene and the pUC polylinker (Rothstein et al. (1987) Gene 53:153-161), and the XhoI-digested fragment are cloned into SalI-digested pTJS75kan to create pCIB200 (see also EP 0 332 104, herein incorporated by reference).

pCIB200 contains the following unique polylinker restriction sites: EcoRI, SstI, KpnI, BglII, XbaI, and SalI. pCIB2001 is a derivative of pCIB200 created by the insertion into the polylinker of additional restriction sites. Unique restriction sites in the polylinker of pCIB2001 are EcoRI, SstI, KpnI, BglII, XbaI, SalI, MluI, BclI, AvrII, ApaI, HpaI, and Stul. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

pCIB10 and Hycromycin Selection Derivatives thereof. The binary vector pCIB10 contains a gene encoding kanamycin resistance for selection in plants and T-DNA right and left border sequences and incorporates sequences from the wide host-range plasmid pRK252 allowing it to replicate in both E. coli and Agrobacterium. Its construction is described by Rothstein et al. (1987). Various derivatives of pCIB 10 are constructed which incorporate the gene for hygromycin B phosphotransferase described by Gritz et al. (1983) Gene 25:179-188. These derivatives enable selection of transgenic plant cells on hygromycin only (pCIB743), or hygromycin and kanamycin (pCIB715, pCIB717).

### VIII.B. Other Plant Transformation Vectors

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Below, the construction of typical vectors suitable for non-Agrobacterium transformation is described.

pCIB3064. pCIB3064 is a pUC-derived vector suitable for direct gene transfer techniques in combination with selection by the herbicide basta (or phosphinothricin). The plasmid pCIB246 comprises the CaMV 35S promoter in operational fusion to the E. coli GUS gene and the CaMV 35S transcriptional terminator and is described in the Internation Publication No. WO 93/07278. The 35S promoter of this vector contains two ATG sequences 5′ of the start site. These sites are mutated using standard PCR techniques in such a way as to remove the ATGs and generate the restriction sites SspI and PvuII. The new restriction sites are 96 and 37 bp away from the unique SalI site and 101 and 42 bp away from the actual start site. The resultant derivative of pCIB246 is designated pCIB3025.

The GUS gene is then excised from pCIB3025 by digestion with SalI and SacI, the termini rendered blunt and religated to generate plasmid pCIB3060. The plasmid pJIT82 is obtained from the John Innes Centre, Norwich and the a 400 bp SmaI fragment containing the bar gene from Streptomyces viridochromogenes is excised and inserted into the HpaI site of pCIB3060 (Thompson et al. (1987) EMBO J. 6:2519-2523). This generated pCIB3064, which comprises the bar gene under the control of the CaMV 35S promoter and terminator for herbicide selection, a gene for ampicillin resistance (for selection in E. coli) and a polylinker with the unique sites SphI, PstI, HindIII, and BamHI. This vector is suitable for the cloning of plant expression cassettes containing their own regulatory signals.

pSOG19 and pSOG35. pSOG35 is a transformation vector that utilizes the E. coli gene dihydrofolate reductase (DFR) as a selectable marker conferring resistance to methotrexate. PCR is used to amplify the 35S promoter (−800 bp), intron 6 from the maize Adhl gene (−550 bp) and 18 bp of the GUS untranslated leader sequence from pS0G10. A 250-bp fragment encoding the E. coli dihydrofolate reductase type II gene is also amplified by PCR and these two PCR fragments are assembled with a SacI-PstI fragment from pB1221 (Clontech, Palo Alto, Calif.) which comprises the pUC19 vector backbone and the nopaline synthase terminator. Assembly of these fragments generates pSOG 19 which contains the 35S promoter in fusion with the intron 6 sequence, the GUS leader, the DHFR gene and the nopaline synthase terminator. Replacement of the GUS leader in pS0G19 with the leader sequence from Maize Chlorotic Mottle Virus (MCMV) generates the vector pSOG35. pS0G19 and pSOG35 carry the pUC gene for ampicillin resistance and have HindIII, SphI, PstI and EcoRI sites available for the cloning of foreign substances.

### VIII.C. Selectable Markers

For certain target species, different antibiotic or herbicide selection markers can be preferred. Selection markers used routinely in transformation include the nptII gene, which confers resistance to kanamycin and related antibiotics (Messing & Vierra (1982) Gene 19:259-268; Bevan et al., 1983), the bar gene, which confers resistance to the herbicide phosphinothricin (White et al. (1990) Nucl Acids Res 18:1062; Spencer et al. (1990) Theor Appl Genet. 79:625-631), the hph gene, which confers resistance to the antibiotic hygromycin (Blochlinger & Diggelmann (1984) Mol Cell Biol 4:2929-2931), the dhfr gene, which confers resistance to methatrexate (Bourouis et al., (1983) EMBO J. 2(7):1099-1104), and the EPSPS gene, which confers resistance to glyphosate (U.S. Pat. Nos. 4,940,935 and 5,188,642).

### IX. Recombinant Expression B Host Cells

The term “host cell”, as used herein, refers to a cell into which a heterologous nucleic acid molecule has been introduced. Transformed cells, tissues, or organisms are understood to encompass not only the end product of a transformation process, but also transgenic progeny thereof. A host cell strain can be chosen which modulates the expression of the inserted sequences, or modifies and processes the gene product in the specific fashion desired. For example, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. Expression in a bacterial system can be used to produce a non-glycosylated core protein product. Expression in yeast will produce a glycosylated product. Expression in plant cells can be used to ensure “native” glycosylation of a heterologous protein.

The present invention provides methods for recombinant expression of FRR/CN/SDS resistance genes in plants by the construction of transgenic plants. The phrase “a plant, or parts thereof” as used herein shall mean an entire plant; and shall mean the individual parts thereof, including but not limited to seeds, leaves, stems, and roots, as well as plant tissue cultures. Transgenic plants of the present invention are understood to encompass not only the end product of a transformation method, but also transgenic progeny thereof. The term “converted plant” as used herein shall mean any plant (1) having resistance to SDS or resistance to SCN and (2) and was derived by genetic selection employing sequence data for at least one of the genes herein defined.

Preferably, the plant is a soybean plant. However, disease resistance can be conferred to a wide variety of plant cells, including those of gymnosperms, monocots, and dicots. Although the gene can be inserted into any plant cell falling within these broad classes, it is particularly useful in crop plant cells, such as rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, tobacco, tomato, sorghum and sugarcane.

### X. Recombinant Expression B Transfection and Transformation Methods

Expression constructs are transfected into a host cell by a standard method suitable for the selected host, including electroporation, calcium phosphate precipitation, DEAE-Dextran transfection, liposome-mediated transfection, infection using a retrovirus, transposon-mediated transfer, and particle bombardment techniques. The FRR/CN/SDS resistance gene-encoding nucleotide sequence carried in the expression construct can be stably integrated into the genome of the host or it can be present as an extrachromosomal molecule. Below are descriptions of representative techniques for transforming both dicotyledonous and monocotyledonous plants.

### X.A. Transformation of Dicotyledons

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al. (1984) EMBO J. 3:2717-2722; Potrykus et al. (1985) Mol Gen Genet. 199:169-177; Reich et al. (1986) Biotechnology 4:1001-1004; and Klein et al. (1987) Nature 327:70-73. In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain, which can depend of the complement of vir genes carried by the host Agrobacterium strain either on a co-resident Ti plasmid or chromosomally (e.g. strain CIB542 for pCIB200 and pCIB2001 (Uknes et al. (1993) Plant Cell 5:159-169). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer (1988) Nucl Acids Res 16:9877).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

### X.B. Transformation of Monocotyledons

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e. co-transformation) and both these techniques are suitable for use with this invention. Co-transformation can have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. (1986) Biotechnology 4:1093-1096).

Patent application Nos. EP 0 292 435, EP 0 392 225, and International Publication No. WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (1990) Plant Cell 2:603-618 and Fromm et al. (1990) Biotechnology 8:833-839 have published techniques for transformation of A188-derived maize line using particle bombardment. Furthermore, International Publication No. WO 93/07278 and Koziel et al. (1993) Biotechnology 11:194-200 describe techniques for the transformation of elite inbred lines of maize by particle bombardment. This technique utilizes immature maize embryos of 1.5-2.5 mm length excised from a maize ear 14-15 days after pollination and a PDS-1000He BIOLISTICS® device for bombardment.

Transformation of rice can also be undertaken by direct gene transfer techniques utilizing protoplasts or particle bombardment. Protoplast-mediated transformation has been described for Japonica-types and Indica-types (Zhang et al. (1988) Plant Cell Rep 7:379-384; Shimamoto et al. (1989) Nature 338:274-277; Datta et al. (1990) Biotechnology 8:736-740). Both types are also routinely transformable using particle bombardment (Christou et al. (1991) Biotechnology 9:957-962). Furthermore, Internation Publication Number WO 93/21335 describes techniques for the transformation of rice via electroporation. Patent application EP 0 332 581 describes techniques for the generation, transformation and regeneration of Pooideae protoplasts. These techniques allow the transformation of Dactylis and wheat. Furthermore, wheat transformation has been described by Vasil et al. (1992) Biotechnology 10:667-674 using particle bombardment into cells of type C long-term regenerable callus, and also by Vasil et al. (1993) Biotechnology 11:1553-1558 and Weeks et al. (1993) Plant Physiol 102:1077-1084 using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog (1962) Physiologia Plantarum 15:473-497) and 3 mg/l 2,4-D for induction of somatic embryos, which is allowed to proceed in the dark. On the chosen day for bombardment, embryos are removed from the induction medium and placed onto the osmoticum (i.e. induction medium with sucrose or maltose added at the desired concentration, typically 15%). The embryos are allowed to plasmolyze for 2-3 h and are then bombarded. Twenty embryos per target plate is typical, although not critical.

An appropriate gene-carrying plasmid (such as pCIB3064 or pSG35) is precipitated onto micrometer size gold particles using standard procedures. Each plate of embryos is shot with the DuPont BIOLISTICS® helium device using a burst pressure of about 1000 psi using a standard 80 mesh screen. After bombardment, the embryos are placed back into the dark to recover for about 24 hours (still on osmoticum). After 24 hours, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

More recently, transformation of monocotyledons using Agrobacterium has been described. See WO 94/00977 and U.S. Pat. No. 5,591,616, both of which are incorporated herein by reference.

### XI. Antibodies

The present invention also provides an antibody immunoreactive with an FRR/CN/SDS resistance polypeptide. The term “antibody” indicates an immunoglobulin protein, or functional portion thereof, including a polyclonal antibody, a monoclonal antibody, a chimeric antibody, a single chain antibody, Fab fragments, and an Fab expression library. “Functional portion” refers to the part of the protein that binds a molecule of interest. In a preferred embodiment, an antibody of the invention is a monoclonal antibody. Techniques for preparing and characterizing antibodies are well known in the art (See, e.g., Harlow and Lane (1988). A monoclonal antibody of the present invention can be readily prepared through use of well-known techniques such as the hybridoma techniques exemplified in U.S. Pat. No. 4,196,265 and the phage-displayed techniques disclosed in U.S. Pat. No. 5,260,203.

The phrase “specifically (or selectively) binds to an antibody”, or “specifically (or selectively) immunoreactive with”, when referring to a protein or peptide, refers to a binding reaction which is determinative of the presence of the protein in a heterogeneous population of proteins and other biological materials. Thus, under designated immunoassay conditions, the specified antibodies bind to a particular protein and do not show significant binding to other proteins present in the sample. Specific binding to an antibody under such conditions can require an antibody that is selected for its specificity for a particular protein. For example, antibodies raised to a protein with an amino acid sequence encoded by the nucleic acid sequence of SEQ ID NO: 1 can be selected to obtain antibodies specifically immunoreactive with that protein and not with unrelated proteins. Such an antibody was raised to SEQ ID NO: 32 Afzal and Lightfoot (2009) Protein Expr Purif 53: 346-355.

The use of a molecular cloning approach to generate antibodies, particularly monoclonal antibodies, and more particularly single chain monoclonal antibodies, are also provided. The production of single chain antibodies has been described in the art. See, e.g., U.S. Pat. No. 5,260,203. For this approach, combinatorial immunoglobulin phagemid libraries are prepared from RNA isolated from the spleen of the immunized animal, and phagemids expressing appropriate antibodies are selected by panning on endothelial tissue. The advantages of this approach over conventional hybridoma techniques are that approximately 10⁴ times as many antibodies can be produced and screened in a single round, and that new specificities are generated by heavy (H) and light (L) chain combinations in a single chain, which further increases the chance of finding appropriate antibodies. Thus, an antibody of the present invention, or a “derivative” of an antibody of the present invention, pertains to a single polypeptide chain binding molecule which has binding specificity and affinity substantially similar to the binding specificity and affinity of the light and heavy chain aggregate variable region of an antibody described herein.

The term “immunochemical reaction”, as used herein, refers to any of a variety of immunoassay formats used to detect antibodies specifically bound to a particular protein, including but not limited to, competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (e.g., using colloidal gold, enzyme or radioisotope labels), western blots, precipitation reactions, agglutination assays (e.g., gel agglutination assays, hemagglutination assays), complement fixation assays, immunofluorescence assays, protein A assays, and immunoelectrophoresis assays, etc. See Harlow and Lane (1988) for a description of immunoassay formats and conditions.

### XII. Method for Detecting a FRR/CN/SDS Resistance Polypeptide

In another aspect of the invention, a method is provided for detecting a level of FRR/CN/SDS resistance polypeptide using an antibody that specifically recognizes a FRR/CN/SDS resistance polypeptide, or portion thereof. In a preferred embodiment, biological samples from an experimental plant and a control plant are obtained, and FRR/CN/SDS resistance polypeptide is detected in each sample by immunochemical reaction with the FRR/CN/SDS resistance polypeptide antibody. More preferably, the antibody recognizes amino acids of SEQ ID NO: 3 and 32 and is prepared according to a method of the present invention for producing such an antibody (Afzal and Lightfoot (2009) Protein Expr Purif 53: 346-355).

In one embodiment, a FRR/CN/SDS resistance polypeptide antibody is used to screen a biological sample for the presence of a FRR/CN/SDS resistance polypeptide. A biological sample to be screened can be a biological fluid such as extracellular or intracellular fluid, or a cell or tissue extract or homogenate. A biological sample can also be an isolated cell (e.g., in culture) or a collection of cells such as in a tissue sample. A tissue sample can be suspended in a liquid medium or fixed onto a solid support such as a microscope slide. In accordance with a screening assay method, a biological sample is exposed to an antibody immunoreactive with an FRR/CN/SDS resistance polypeptide whose presence is being assayed, and the formation of antibody-polypeptide complexes is detected. Techniques for detecting such antibody-antigen conjugates or complexes are well known in the art and include but are not limited to centrifugation, affinity chromatography and the like, and binding of a labeled secondary antibody to the antibody-candidate receptor complex.

### XIII. Identification of Modulators of FRR/CN/SDS Resistance

The present invention further discloses a method for identifying a compound that modulates FRR/CN/SDS resistance. As used herein, the terms “candidate substance” and “candidate compound” are used interchangeably and refer to a substance that is believed to interact with another moiety, wherein a biological activity is modulated. For example, a representative candidate compound is believed to interact with a complete, or a fragment of, a FRR/CN/SDS resistance polypeptide, and which can be subsequently evaluated for such an interaction. Exemplary candidate compounds that can be investigated using the methods of the present invention include, but are not restricted to, compounds that confer FRR/CN/SDS resistance, viral epitopes, peptides, enzymes, enzyme substrates, co-factors, lectins, sugars, oligonucleotides or nucleic acids, oligosaccharides, proteins, chemical compounds small molecules, and monoclonal antibodies. A candidate compound to be tested by these methods can be a purified molecule, a homogenous sample, or a mixture of molecules or compounds.

As used herein, the term “modulate” means an increase, decrease, or other alteration of any or all chemical and biological activities or properties of a wild-type FRR/CN/SDS resistance polypeptide, preferably a FRR/CN/SDS resistance polypeptide of SEQ ID NO: 3. Preferably, a FRR/CN/SDS resistance modulator is an agonist of FRR/CN/SDS resistance protein activity. As used herein, the term “agonist” means a substance that supplements or potentiates the biological activity of a functional FRR/CN/SDS resistance protein.

In accordance with the present invention there is also provided a rapid and high throughput screening method that relies on the methods described above. This screening method comprises separately contacting each compound with a plurality of substantially identical samples. In such a screening method the plurality of samples preferably comprises more than about 10⁴ samples, or more preferably comprises more than about 5×10⁴ samples. In an alternative high-throughput strategy, each sample can be contacted with a plurality of candidate compounds.

### XIII.A. Methods for Identifying Modulators of FRR/CN/SDS Resistance Gene Expression

The nucleic acid sequences of the present invention can be used to identify regulators of FRR/CN/SDS resistance polypeptide gene expression. Several molecular cloning strategies can be used to identify substances that specifically bind FRR/CN/SDS resistance polypeptide cis-regulatory elements. A preferred promoter region to be used in such assays is an FRR/CN/SDS resistance polypeptide promoter region from soybean, more preferably the promoter region includes some or all amino acids of SEQ ID NO: 3.

In one embodiment, a cDNA library in an expression vector, such as the lambda-gt11 vector, can be screened for cDNA clones that encode an FRR/CN/SDS resistance polypeptide regulatory element DNA-binding activity by probing the library with a labeled FRR/CN/SDS resistance polypeptide DNA fragment, or synthetic oligonucleotide (Singh et al. (1989) Biotechniques 7:252-261). Preferably the nucleotide sequence selected as a probe has already been demonstrated as a protein binding site using a protein-DNA binding assay described above.

In another embodiment, transcriptional regulatory proteins are identified using the yeast one-hybrid system (Luo et al. (1996) Biotechniques 20(4):564-568; Vidal et al. (1996) Proc Natl Acad Sci USA 93(19):10315-10320; Li and Herskowitz (1993) Science 262:1870-1874). In this case, a cis-regulatory element of a FRR/CN/SDS resistance gene is operably fused as an upstream activating sequence (UAS) to one, or typically more, yeast reporter genes such as the lacZ gene, the URA3 gene, the LEU2 gene, the HIS3 gene, or the LYS2 gene, and the reporter gene fusion construct(s) is inserted into an appropriate yeast host strain. It is expected that the reporter genes are not transcriptionally active in the engineered yeast host strain, for lack of a transcriptional activator protein to bind the UAS derived from the FRR/CN/SDS resistance gene promoter region. The engineered yeast host strain is transformed with a library of cDNAs inserted in a yeast activation domain fusion protein expression vector, e.g. pGAD, where the coding regions of the cDNA inserts are fused to a functional yeast activation domain coding segment, such as those derived from the GAL4 or VP16 activators. Transformed yeast cells that acquire a cDNA encoding a protein that binds a cis-regulatory element of a FRR/CN/SDS resistance gene can be identified based on the concerted activation the reporter genes, either by genetic selection for prototrophy (e.g. LEU2, HIS3, or LYS2 reporters) or by screening with chromogenic substrates (lacZ reporter) by methods known in the art.

The present invention also provides an in vivo assay for discovery of modulators of FRR/CN/SDS resistance gene expression. In this case, a transgenic plant is made such that a transgene comprising a FRR/CN/SDS resistance gene promoter and a reporter gene is expressed and a level of reporter gene expression is assayable. Such transgenic animals can be used for the identification of compounds that are effective in modulating FRR/CN/SDS resistance gene expression.

In vitro or in vivo screening approaches may survey more than one modulatable transcriptional regulatory sequence simultaneously.

### XIII.B. Methods for Identifying Modulators of FRR/CN/SDS Resistance Polypeptides

According to the method, a FRR/CN/SDS resistance polypeptide is exposed to a plurality of candidate substances, and binding of a candidate substance to the FRR/CN/SDS resistance polypeptide is assayed. A compound is selected that demonstrates specific binding to the FRR/CN/SDS resistance polypeptide. Preferably, the FRR/CN/SDS resistance polypeptide used in the binding assay of the method includes some or all amino acids of SEQ ID NO: 3.

The term “binding” refers to an affinity between two molecules, for example, a ligand and a receptor, means a preferential binding of one molecule for another in a mixture of molecules. The binding of the molecules can be considered specific if the binding affinity is about 1×10⁴ M⁻¹ to about 1×10⁶ M⁻¹ or greater. Binding of two molecules also encompasses a quality or state of mutual action such that an activity of one protein or compound on another protein is inhibitory (in the case of an antagonist) or enhancing (in the case of an agonist).

Several techniques can be used to detect interactions between a protein and a chemical ligand without employing an in vivo ligand. Representative methods include, but are not limited to, fluorescence correlation spectroscopy, surface-enhanced laser desorption/ionization, and biacore technology, each described herein below. These methods are amenable to automated, high-throughput screening.

Fluorescence Correlation Spectroscopy (FCS). FCS measures the average diffusion rate of a fluorescent molecule within a small sample volume (Madge et al. (1972) Phys Re Lett 29:705-708, Maiti et al. (1997) Proc Natl Acad Sci USA, 94:11753-11757). The sample size can be as low as 10³ fluorescent molecules and the sample volume as low as the cytoplasm of a single bacterium. The diffusion rate is a function of the mass of the molecule and decreases as the mass increases. FCS can therefore be applied to protein-ligand interaction analysis by measuring the change in mass and therefore in diffusion rate of a molecule upon binding. In a typical experiment, the target to be analyzed is expressed as a recombinant protein with a sequence tag, such as a poly-histidine sequence, inserted at the N-terminus or C-terminus. The target protein is expressed in E. coli, yeast, or plant cells. The protein is purified by chromatography. For example, the poly-histidine tag can be used to bind the expressed protein to a metal chelate column such as Ni²⁺ chelated on iminodiacetic acid agarose. The protein is then labeled with a fluorescent tag such as carboxytetramethylrhodamine or BODIPY™ (Molecular Probes, Eugene, Oreg.). The protein is then exposed in solution to a candidate compound, and its diffusion rate is determined by FCS, using for example, instrumentation available from Carl Zeiss, Inc. (Thornwood, N.Y.). Ligand binding is determined by changes in the diffusion rate of the protein.

Surface-Enhanced Laser Desorption/lonization (SELDI). SELDI can be used in combination with a time-of-flight mass spectrometer (TOF) to provide a means to rapidly analyze molecules retained on a chip (Hutchens and Yip (1993) Rapid Commun Mass Spectrom 7:576-580). It can be applied to ligand-protein interaction analysis by covalently binding the target protein on the chip and using mass spectroscopy to analyze the small molecules that bind to the target protein (Worrall et al. (1998) Anal Biochem 70:750-756). In a typical experiment, the target to be analyzed is recombinantly expressed, optionally with a tag, such as poly-histidine, to facilitate purification and handling. The purified protein is bound to the SELDI chip either by utilizing the poly-histidine tag or by other interaction such as ion exchange or hydrophobic interaction. The chip thus prepared is then exposed to a candidate compound via, for example, a delivery system able to pipet the ligands in a sequential manner (autosampler). The chip is then washed in buffers of increasing stringency, for example a series of buffer solutions containing incrementally increasing ionic strength. After each wash, the bound material is analyzed by SELDI-TOF. Compounds that specifically bind the target are identified by elution in high stringency washes.

Biacore. Biacore technology utilizes changes in the refractive index at the surface layer upon binding of a ligand to a protein immobilized on the layer. In this system, a collection of small ligands is injected sequentially in a 2-5 microliter cell, wherein the protein is immobilized within the cell. Binding is detected by surface plasmon resonance (SPR) of laser light refracting from the surface. In general, the refractive index change for a given change of mass concentration at the surface layer is practically the same for all proteins and peptides, allowing a single method to be applicable for any protein (Liedberg et al. (1983) Sensors Actuators 4:299-304; Malmquist (1993) Nature 361:186-187). In a typical experiment, the target protein to be analyzed is recombinantly expressed and purified according to standard methods (Afzal and Lightfoot 2009 Protein Expr Purif 53: 346-355. It is bound to the Biacore chip either by utilizing a poly-histidine tag or by other interaction such as ion exchange or hydrophobic interaction. The chip thus prepared is then exposed to a candidate compound via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics on rate and off rate allows the discrimination between non-specific and specific interaction.

Rational Drug Design. Similarly, the knowledge of the structure a native FRR/CN/SDS resistance polypeptide provides an approach for rational drug design. The structure of an FRR/CN/SDS resistance polypeptide can be determined by X-ray crystallography or by computational algorithms that generate three-dimensional representations. See Huang et al. (2000) and Saqi et al. (1999) Computer models can further predict binding of a protein structure to various substrate molecules, that can be synthesized and tested. Additional drug design techniques are described in U.S. Pat. Nos. 5,834,228 and 5,872,011.

### XIV. Modulation of FRR/CN/SDS Resistance in a Plant

In accordance with the present invention a method of modulating FRR/CN/SDS resistance in a plant is also provided. The method comprises the step of administering to the plant an effective amount of a substance that modulates expression of an FRR/CN/SDS resistance activity-encoding nucleic acid molecule in the plant to thereby modulate FRR/CN/SDS resistance in the plant. Preferably, the substance that modulates expression of an FRR/CN/SDS resistance activity-encoding nucleic acid molecule comprises a ligand for a modulatable transcriptional regulatory sequence of an FRR/CN/SDS resistance activity-encoding nucleic acid molecule identified in accordance with the methods described above. More preferably, the plant is a soybean plant.

Particularly, provided chemical entities (e.g. small molecule mimetics) do not naturally occur in any cell of a lower eucaryotic organism such as yeast. More particularly, provided chemical entities do not naturally occur in any cell, whether of a multicellular or a unicellular organism. Even more particularly, the provided chemical entity is not a naturally occurring molecule, e.g. it is a chemically synthesized entity. Provided chemical entities can be hydrophobic, polycyclic, or both, molecules, and are typically about 500-1,000 daltons in molecular weight.

### XV. Method for Providing FRR/CN/SDS Resistance B Transgenic Plants

A “transgenic plant” is a plant that has been genetically modified to contain and express heterologous DNA sequences, either as regulatory RNA molecules or as proteins. As specifically exemplified herein, a transgenic plant is genetically modified to contain and express at least one heterologous DNA sequence operably linked to and under the regulatory control of transcriptional control sequences which function in plant cells or tissue or in whole plants. As used herein, a transgenic plant also refers to progeny of the initial transgenic plant where those progeny contain and are capable of expressing the heterologous coding sequence under the regulatory control of the plant-expressible transcription control sequences described herein. Seeds containing transgenic embryos are encompassed within this definition as are cuttings and other plant materials for vegetative propagation of a transgenic plant.

When plant expression of a heterologous gene or coding sequence of interest is desired, that coding sequence is operably linked in the sense orientation to a suitable promoter and advantageously under the regulatory control of DNA sequences which quantitatively regulate transcription of a downstream sequence in plant cells or tissue or in planta, in the same orientation as the promoter, so that a sense (i.e., functional for translational expression) mRNA is produced. A transcription termination signal, for example, as polyadenylation signal, functional in a plant cell is advantageously placed downstream of the FRR/CN/SDS resistance coding sequence, and a selectable marker which can be expressed in a plant, can be covalently linked to the inducible expression unit so that after this DNA molecule is introduced into a plant cell or tissue, its presence can be selected and plant cells or tissue not so transformed will be killed or prevented from growing.

In the present invention, the FRR/CN/SDS resistance coding sequence can optionally serve as a selectable marker for transformation of plant cells or tissue. Where constitutive gene expression is desired, suitable plant-expressible promoters include a native promoter (e.g. SEQ ID NO:15) of the FRR/CN/SDS coding sequences set forth herein as the native promoter is activated in the presence of SCN; the 35S or 19S promoters of Cauliflower Mosaic Virus; the nos, ocs or mas promoters of Agrobacterium tumefaciens Ti plasmids; and others known to the art.

Indeed, a native promoter (e.g. SEQ ID NO:2 or 4) of the FRR/CN/SDS coding sequences set forth herein is activated in the presence of SCN and thus can be used to produce transgenic plants in accordance with the techniques disclosed herein. Particularly, the native promoter can be linked to a nucleic acid encoding a polypeptide of interest in a construct, and the construct can be used to a prepare a transgenic plant in accordance with techniques described herein. Other techniques are disclosed in U.S. Pat. Nos. 5,994,526 and 5,994,527, herein incorporated by reference in their entirety. The polypeptide of interest is then expressed in the plant when the promoter is activated, such as in the presence of SCN or other environmental stimulus.

Where tissue-specific expression of the FRR/CN/SDS resistance coding sequence is desired, the skilled artisan will choose from a number of well-known sequences to mediate that form of gene expression as disclosed herein. Environmentally regulated promoters are also well known in the art, and the skilled artisan can choose from well known transcription regulatory sequences to achieve the desired result.

A method for providing a resistance characteristic to a plant is therefore disclosed. The method comprises introducing to said plant a construct comprising a nucleic acid sequence encoding an FRR/CN/SDS resistance gene product operatively linked to a promoter, wherein production of the FRR/CN/SDS resistance gene product in the plant provides a resistance characteristic to the plant. The construct can further comprises a vector selected from the group consisting of a plasmid vector or a viral vector. The FRR/CN/SDS resistance gene product comprises a protein having an amino acid sequence as set forth as SEQ ID NO:3. The nucleic acid sequence can be a nucleic acid sequence set forth as SEQ ID NO:1, 2, 4 or 32 or a nucleic acid that is substantially similar to SEQ ID NO: 1, 2, 4 or 32, and which encodes an FRR/CN/SDS resistance polypeptide.

The resistance characteristic is preferably nematode resistance, fungal resistance or combinations thereof. More preferably, the nematode resistance is H. glycines resistance or root knot nematode resistance.

In an alternative embodiment, the construct further comprises another nucleic acid molecule encoding a polypeptide that provides an additional desired characteristic to the plant. Other desired characteristics include yield, drought resistance, chemical resistance (e.g. herbicide or pesticide resistance), spoilage resistance or any or other desired characteristic as would be apparent to one of ordinary skill in the art after review of the disclosure of the present invention. Representative nucleic acids sequences are described in the following U.S. patents (incorporated herein by reference in their entirety): U.S. Pat. No. 5,948,953 to Webb (brown stem rot fungus resistance); U.S. Pat. No. RE36,449 to Lebrun et al. (herbicide resistance); U.S. Pat. No. 5,952,546 to Bedbrook et al. (delayed ripening tomato plants); U.S. Pat. No. 7,154,021 to Hauge et al.; and U.S. Pat. No. 5,986,173 to Smeekens et al. (transgenic plants showing a modified fructan pattern).

Optionally, the method further comprises monitoring an insertion point for the construct in the plant genome; and providing for insertion of the construct into the plant genome at a location not associated with the resistance characteristic, the desired characteristic, or both the resistance or the desired characteristic.

XVI. Method for Providing FRR/CN/SDS Resistance B Marker-Assisted Selection and development of a Breeding Program

The present invention relates to a novel and useful method for introgressing, in a reliable and predictable manner, FRR/CN/SDS resistance into non-resistant soybean germplasm. The method involves the genetic mapping of loci associated with FRR/CN/SDS resistance, definition of genetic markers that are linked with FRR/CN/SDS resistance, and a high-throughput PCR-based assay for detecting such a genetic marker. Markers useful in a preferred embodiment of the invention include the following: a locus mapping to linkage group G and mapped by one or more of the markers set forth SEQ ID NOs: 4, or combinations thereof. Also preferably, a genetic marker used for marker-assisted selection comprises a sequence, or portion thereof, of any one of SEQ ID NOs:1, 2, 4 and 32, or combinations thereof.

From the sequence data found in SEQ ID NOs: 1, 2, 4 and 32, and from the other markers identified herein, primer pairs, as for example, PCR primer pairs, capable of distinguishing differences among these genotypes are developed. Simple assays for the markers and genes use a label, such as, but not limited to, a covalently attached chromophores, that do not need electrophoresis are developed to increase the capacity of marker assisted selection to help plant breeders. A preferred assay is the TaqMan™ assay disclosed in Examples. Non-destructive sampling of dried seed for DNA preparations are developed to allow selection prior to planting, for example, using the methods set forth in Examples. This enables the testing of the effectiveness of marker assisted selection in predicting field resistance to SCN and SDS.

A preferred manner for providing FRR/CN/SDS resistance to a plant involves providing one or more plants from a parental soybean plant line which comprises in its genome one or more molecular markers comprising a sequence, or portion thereof, set forth as any one of SEQ ID NOs: 1, 2, 4 and 32. Preferably, the parental plant is pure-breeding for one or more of the molecular markers, more preferably the parent plant is pure-breeding for molecular markers comprising a sequence, or portion thereof, set forth as any one of SEQ ID NOs: 1, 2, 4 and 32. In one preferred embodiment, the parental line is “Forrest” or a line derived therefrom.

The FRR/CN/SDS resistance trait can be introgressed into a recipient soybean plant line which is non-resistant or less resistant to FRR/CN/SDS by performing marker-assisted selection based on the molecular markers of the present invention as set forth as SEQ ID NOs: 1, 2, 4 and 32.

Introgressing can be accomplished by any method known in the art, including but not limited to single seed descent, pedigree method, or backcrossing, each described herein below. Additional methods for introgressing are disclosed in U.S. Pat. Nos. 5,948,953 and 6,162,967. Any suitable method can be used, the critical feature being marker-assisted selection of a marker of the present invention using a nucleotide sequence assay.

Single Seed Descent. According to this method, “Forrest” can be crossed to “Essex”, and the seed planted in a field. The resulting seed (F2) is planted in the greenhouse and the resulting seeds (F3) are harvested while keeping separate the seeds from each plant. A random F3 seed from each of approximately 200 plants is planted and the resulting F4 seed is harvested. The seeds from each individual plant are again kept separate. A random F4 seed from each of the approximately 200 plants is planted and the resulting F5 seed is harvested. This selection process is repeated until F7 seed is harvested and identified as an inbred line. At each generation beginning with the F3 generation, plants are screened with soybean cyst nematodes, and plants were selected for advancement based upon the presence of SCN resistance and other phenotypic characteristics. Alternatively, plants are screened for the presence of one or more of the molecular markers listed herein using a TaqMan™ genotyping assay and selected for advancement based upon the presence of one or more of the markers.

Pedigree Method. Using a SCN resistant recombinant inbred line, produced for example by single seed descent, as a donor source, the SCN resistant trait can be introgressed into other germ plasm sources. To develop new germplasm, the SCN resistant recombinant inbred line is used as one of the parents. The resulting progenies are evaluated and selected at various locations for a variety of traits, including SCN resistance. SCN resistance is determined by phenotypic screening or by genotyping based upon the presence of the molecular markers listed herein.

Backcrossing. Using a SCN resistant recombinant inbred line, produced for example by single seed descent, as a donor source, the SCN resistant trait is introgressed into other soybean plant lines. The SCN resistant recombinant inbred line is crossed to a line that demonstrates little or non SCN resistance (the recipient). The resulting plants are crossed back to the recipient soybean plant line that is being converted to SCN resistance. This crossing back to the parental line that is being converted may be repeated several times. After each round of backcrossing, plants are selected for SCN resistance, which can be determined by either phenotypic screening or by the selection of molecular markers linked to SCN resistance loci. Besides selecting for SCN resistance, the plants are also selected that most closely resemble the original plant line being converted to SCN resistance. This selection for the original plant line is done phenotypically or with molecular markers.

In one specific preferred method, BC_{NF1} plants are genotypically screened for the presence of one or more markers linked to SCN resistance genomic loci. As used herein, the term “BC_{NF1}plant” is intended to refer to a plant in the first generation after a specific backcross event, the specific backcross event being designated by the term “N”, irrespective of the number of previous backcross events employed to produce the plant. Plants having the one or more markers present may preferably be backcrossed with plants of the parental line or, alternatively, be selfed, the plants resulting from either of these events also being genotypically screened for the presence of one or more markers linked to SCN resistance genomic loci. This procedure can be repeated several times.

In another specific preferred method, BC_{NF1}plants are selfed to produce BC_{NF2} seeds. BC_{NF2} plants are then screened either genotypically using, for example a TaqMan™ assay as disclosed in Example 6, or by phenotypic assessment of SCN resistance. Those plants having present one or more molecular markers linked to SCN resistance, or those plants displaying resistance, depending upon the screening method used, are backcrossed with plants of the parental line to produce BC_{NF3} seeds and plants. This procedure can be repeated several times. In a soybean breeding program, the methods of the present invention can be used for marker-assisted selection of the molecular markers described herein. Genetic markers closely linked to FRR/CN/SDS resistance genes can be used to indirectly select for favorable alleles more efficiently than phenotypic selection. Genetic markers comprising FRR/CN/SDS resistance genes, as disclosed herein, can be used to select for FRR/CN/SDS resistance genes with optimal efficiency and accuracy.

Marker-assisted selection can be employed to select one or more loci at a wide variety of population development stages in a two-parent population, multiple parent population, or a backcross population. Such populations are described in Fehr (1987) Breeding Methods for Cultivar Development J. R. Wilcox (ed.) and Soybeans: Improvement, Production, and Uses, 2nd ed.

Marker-assisted selection according to art-recognized methods can be made, for example, step-wise, whereby the different SCN resistance loci are selected in more than one generation; or, as an alternative example, simultaneously, whereby all loci are selected in the same generation. Marker-assisted selection for SCN resistance can be done before, in conjunction with, or after testing and selection for other traits such as seed yield, plant height, seed type, etc. The DNA from target populations, isolated for use in accordance with genetic marker detection, can be obtained from any plant part, and each DNA sample can represent the genotype of single or multiple plant individuals, including seed.

Marker-assisted selection can also be used to confirm previous selection for SCN resistance or susceptibility made by challenging plants with SCNs in the field or greenhouse and scoring the resulting phenotypes. Alternatively, plants can be analyzed by TaqMan™ genotyping to determine the presence of the above-described molecular markers, thus confirming the presence of a genomic locus associated with SCN resistance.

As such, also provided by the present invention are methods for determining the presence or absence of SCN resistance in a soybean plant, or alternatively in a soybean seed. These methods comprise analyzing genomic DNA from a plant or a seed for the presence of one or more of the molecular markers set forth as SEQ ID NOs:1-13 and 16-19. According to this method, the analyzing comprises performing a TaqMan™ assay as disclosed in Example 6, or any other suitable method known in the art.

The ability to distinguish heterozygotes and their derived heterogeneous lines is important to early generation selection (before the F₅) in soybean breeding programs when within population variability is high (Bernard et al. (1988) USDA Tech Bull 1796; Brown et al., 1987). The lower stringency TaqMan™ 2 assay disclosed herein was most effective for identifying most of the heterogeneous lines in this population. However, the cutoff values of FAM and TET for the efficient identification of heterogeneous lines (or heterozygous F2 lines) is likely to vary across assays and should be set arbitrarily according to expectations of the number of lines that are expected to contain both alleles. The assay was used for analyzing 2,000 lines derived from specific cultivar crosses over 3 days. A single researcher can process 768 sample per day (8×96 samples) since the reading time of the machine is 15 minutes for one 96 well plate and the thermal cycler stage takes about 2 hours.

Summarily, the sequences and methods disclosed herein enable automated, high throughput, rapid genotyping of DNA polymorphisms for selection of FRR/CN/SDS resistance in breeding programs.

### EXAMPLES

The following Examples have been included to illustrate preferred modes of the invention. Certain aspects of the following Examples are described in terms of techniques and procedures found or contemplated by the present inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

### Example 1

### Plant Material

‘Forrest’ (Hartwig & Epps (1973) Crop Sci 13:287; Lightfoot et al., 2005, Crop Sci 45:1678-1681) is resistant to the soybean cyst nematode (SCN) populations classified as race 3. Soybean cultivars ‘X5’ and ‘Westag 97’ were used for transformation because they could be regenerated to plant efficiently from embryo cultures [67]. They were susceptible to both SCN and SDS. They were judged to be rhg1/rfs2rhg1/rfs2, rhg4, rhg4 based on the assays reported here. Crosses were made to RIL EF2 for SCN tests which was rhg1/rfs2, rhg1/rfs2, Rhg4, Rhg4.

### Example 2

### SCN Female Index (FI) Determination

The number of white female cysts was compared on each genotype to the number of white female cysts on a susceptible control, such as Essex, to determine the female index (FI) for each population (Meksem et al., 1999). Seedlings were inoculated with 2000+/−25 eggs from a homogenous isolate of H. glycines. All experiments used five single-plant replications per line. The mean number of white female cysts on each genotype and the susceptible control were determined and Fl was calculated as the ratio of the mean number of cysts on each genotype to the mean number of cysts on the susceptible check. Three SCN populations were used, PA3 JB3 and WL3. The indicator lines FI for nematode population PA3 were Hutcheson 100±0%; Peking 0.5±0.2%; Pickett 0.4±0.1%; PI88788 4±2.0%; PI90763 0.1±0%; Essex 83±5.0%; and Forrest 0.5±0.1% across the experiments. The indicator lines female indices (FI) for nematode population JB3 were ‘PI54840’ (FI 7%), PI 88788 (FI 2%), PI90763 (FI 1%), PI437654 (FI 0%), ‘PI 209332’ (FI 1%), ‘PI89772’ (FI 2%) ‘PI548316’ (FI 8%) and ‘PI548402’ (FI 3%). The soil collected from Yichun in China contained Hg Type 0 (SCN race 3) isolate WL3 was ‘Peking’ (FI 0%), ‘PI 88788’ (FI 0%), ‘PI 90763’ (FI 6%) and ‘Pickett’ (FI 9%). Therefore, the standard differentials showed these HG Types to be all variants on Hg Type 0 (Niblack et al. 2003 J Nematol 35:355-345) corresponding to race 3 (Riggs and Schmitt 1988, J Nematol 20: 392-395).

### Example 3

### SDS Measures Following F. Virguliforme Infestations

Greenhouse assays of SDS followed the methods previously described (Njiti et al. 2001, Crop Sci 41:1726-1733; U.S. Pat. No. 7,288,386) with the following modifications. Isolate Mont 1 of Fusarium virguliforme was grown on potato dextrose agar media for 7 days then tranferred to silica cornmeal media for 14 days. Leaf symptoms were rated every 7 days from infestation to senescence. At 28 days after infection (dai) roots were washed, photographed and a root sample (1 g) taken. Plants were repotted into the media of the reciprocal genotype to test for pot effects. The experiments were repeated on 3 occasions using 3-5 plants of each genotype. Highly resistant plants of line EF23, and highly susceptible plants of line EF85 were assayed in parallel with each test.

### Example 4

### Cloning of FRR/CN/SDS Resistance Genes in Linkage Groups G and B1

The cloned AFLP bands of the allowed US patents (U.S. Pat. No. 6,300,541) and (U.S. Pat. No. 7,902,337) were used to screen the soybean Forrest BamHI or HindIII BAC libraries by PCR as described by Meksem et al. (2000) Theor. Appl. Genet. 101:747-755 to identify clones B73P06 and H21D09. Probes derived from the RLK at Rhg1/Rfs2 were used to identify clone H38F23 as described by Afzal et al., 2008; Nature Preceedings hd1:10101/npre.2008.2726.1.

BACs B73p06 (SEQ ID NO 4) and H38f23 (SEQ ID NO 33) were sequenced at TIGR (nee JCVI). Briefly, the entire BAC was sheared by nebulization to provide fragments in the 3-5 kbp or 9-11 kbp range. The fragments were ligated into pHOS2 and used for Sanger DNA sequencing. BAC DNA was prepared using the appropriate kit (Qiagen, Hilden, Germany). Sequence determinations were performed by the di-deoxy chain-termination method using Advanced Biosystems (ABI, Foster city, Calif.) “big dye” cycle sequencing separated on ABI 3730 automated DNA sequencer. Plasmids containing clones derived from BACs were sequenced using M13 universal forward and reverse primers. The PCR conditions used was 95° C. for 10 min, then 45 cycles of 95° C. for 30 sec, 55° C. for 20 sec and 60° C. for 4 min.

BACs were sequenced to 8-12 fold redundancy and assembled. Assembly quality was judged by BLAST comparisions for sequences from A3244 BACs (Hauge et al. 2006 US patent; Ruben et al. 2006 MGG) and Williams 82 genome sequence (Schmutz et al. 2010). Features and polymorphisms that were unique to the SDS and SCN resistant soybean cultivars are listed in Table 2.

Table 2: Listing of DNA sequence variations (SNPs; indels), cis-regulators elements (enhancers) and protein coding regions in BAC 73P06 from resistant cultivar Forrest compared to susceptible cultivars A3244 and Williams 82. Panel A; complete list of features distinguishing resistant Forrest from the whole BAC. Panel B; features and primers used as markers in the RLK gene at rhg1/Rfs2. Panel C. Primers for Taqman probes to synonymous SNPS in the RLK alleles. Panel D. Primers for Taqman probes to non-synonymous SNPS that change amino acid residues in the RLK alleles. Panel E; Primers for new indels discovered in the region of the RLK by BAC sequencing.

FEATURES Location/Qualifiers misc_feature 1 . . . 82157 /note = “Rhg1 gene locus”

variation 34 /note = “SNP; nongenic” /replace = “t” enhancer 401 . . . 408 variation 791 /note = “SNP;
nongenic” /replace = “a” enhancer 863 . . . 869 enhancer 872 . . . 879 variation 920 /note = “SNP;
nongenic” /replace = “a” variation 978 /note = “SNP; nongenic” /replace = “a” variation 1497
/note = “SNP; nongenic” /replace = “t” enhancer 1524 . . . 1531
enhancer 1579 . . . 1586 enhancer 1605 . . . 1611
variation 1689 /note = “SNP; nongenic” /replace = “t” variation 1745 /note = “SNP; nongenic”
/replace = “t” variation 1757 /note = “SNP; nongenic” /replace = “t” variation 1759 /note = “SNP;
nongenic” /replace = “t” repeat_region 1822 . . . 1829 /rpt_type = tandem
/satellite = “microsatellite:SIUCSat75” variation 1830 /note = “SNP; nongenic” /replace = “t” variation
1841 /note = “SNP; nongenic” /replace = “a” enhancer 1843 . . . 1850 variation 1855 /note = “SNP;
nongenic” /replace = “t” variation 1859 /note = “SNP; nongenic” /replace = “a” variation 1876
/note = “SNP; nongenic” /replace = “t” variation 1883 /note = “SNP; nongenic” /replace = “a” variation
1885 /note = “SNP; nongenic” /replace = “a” variation 1887 /note = “SNP; nongenic” /replace = “a”
variation 1889 /note = “SNP; nongenic” /replace = “a” variation 1898 /note = “SNP; nongenic”
/replace = “g” variation 1902 /note = “SNP; nongenic” /replace = “g” variation 1904 /note = “SNP;
nongenic” /replace = “g” variation 1906 /note = “SNP; nongenic” /replace = “g” variation 1931
/note = “SNP; nongenic” /replace = “c” variation 1963 /note = “SNP; nongenic” /replace = “g” variation
1987 /note = “SNP; nongenic” /replace = “c” variation 2030 /note = “SNP; nongenic” /replace = “a”
variation 2064 /note = “SNP; nongenic” /replace = “t” variation 2093 /note = “SNP; nongenic”
/replace = “t” variation 2279 /note = “SNP; nongenic” /replace = “a” variation 2403 /note = “SNP;
nongenic” /replace = “g” enhancer 2414 . . . 2421 variation 2568 /note = “SNP; nongenic” /replace = “c”
variation 2573 /note = “SNP; nongenic” /replace = “t” variation 2575 /note = “SNP; nongenic”
/replace = “c” enhancer 2655 . . . 2662 enhancer
2665 . . . 2671 enhancer 2697 . . . 2704 enhancer 2700 . . . 2706
enhancer 3003 . . . 3010 variation 3167 /note = “SNP; nongenic” /replace = “g” variation 3188
/note = “SNP; nongenic” /replace = “a” variation 3349 /note = “SNP; nongenic” /replace = “t” variation
3371 /note = “SNP; nongenic” /replace = “t” variation 3471 /note = “SNP; nongenic” /replace = “c”
enhancer 3484 . . . 3490 enhancer 3500 . . . 3507 enhancer
3582 . . . 3589 enhancer 3608 . . . 3617 variation 3983
/note = “SNP; nongenic” /replace = “a” variation 4038 /note = “SNP; nongenic” /replace = “a” variation
4095 /note = “SNP; nongenic” /replace = “c” variation 4126 /note = “SNP; nongenic” /replace = “c”
variation 4188 /note = “SNP; nongenic” /replace = “c” variation 4223 /note = “SNP; nongenic”
/replace = “t” variation 4228 /note = “SNP; nongenic” /replace = “g” enhancer 4264 . . . 4271 variation
4479 /note = “SNP; nongenic” /replace = “c” variation 4560 /note = “SNP; nongenic” /replace = “t”
variation 4594 /note = “SNP; nongenic” /replace = “a” enhancer 4645 . . . 4653 variation 4873
/note = “SNP; nongenic” /replace = “t” variation 4923 /note = “SNP; nongenic” /replace = “a” variation
5062 /note = “SNP; nongenic” /replace = “a” enhancer 5219 . . . 5227 enhancer 5303 . . . 5312 variation
5335 /note = “SNP; nongenic” /replace = “t” enhancer 5418 . . . 5424 variation 5497 /note = “SNP;
nongenic” /replace = “t” variation 5522 /note = “SNP; nongenic” /replace = “g” promoter 5849 . . . 5899
variation 5852 /note = “SNP; nongenic” /replace = “t” variation 5899 /note = “SNP; nongenic”
/replace = “g” mRNA join(<5959 . . . 6125, 6328 . . . 6652, 7346 . . . 7428,
7518 . . . 7587, 7674 . . . 7748, 7852 . . . >7947)
/product = “hypothetical protein” CDS join(5959 . . . 6125,
6328 . . . 6652, 7346 . . . 7428, 7518 . . . 7587,
7674 . . . 7748, 7852 . . . 7947) /note = “similar to
NADP oxidoreductase coenzyme F420-dependent, Pfam number
03807; conserved domain, NADB_Rossmann; Rossmann-fold NAD(P)(+)-binding proteins; cl09931;
similar to INSD accession number ACU23513”
/codon_start = 1 /product = “hypothetical protein” /protein_id = “AET79242.1”
/db_xref = “GI: 357432828”
/translation = “MSTSSSSQSLKIGIVGFGNFGQFLAKTMIKQGHTLTATSRSDYS
ELCLQMGIHFFRDVSAFLTADIDVIVLCTSILSLSEVVGSMPLTSLKRPTLFVDVLSV
KEHPRELLLRELPEDSDILCTHPMFGPQTAKNGWTDHTFMYDKVRIRDQATCSNFIQI
FATEGCKMVQMSCEEHDRAAAKSQFITHTIGRTLGEMDIQSTPIDTKGFETLVKLKET
MMRNSFDLYSGLFVYNRFARQELENLEHALHKVKETLMIQRTNGEQGHKRTES” enhancer 6146 . . . 6153
variation 6202 /note = “SNP; nongenic” /replace = “g” variation 6322 /note = “SNP; nongenic”
/replace = “a” variation 6352 /note = “SNP; genic” /replace = “a” variation 6611 /note = “SNP; genic”
/replace = “g” variation 6615 /note = “SNP; genic” /replace = “t” variation 6618 /note = “SNP; genic”
/replace = “t” variation 6681 /note = “SNP; nongenic” /replace = “t” variation 6730 /note = “SNP;
nongenic” /replace = “g” variation 6791 /note = “SNP; nongenic” /replace = “a” variation 6845
/note = “SNP; nongenic” /replace = “g” variation 6867 /note = “SNP;
nongenic” /replace = “a” variation
6914 /note = “SNP; nongenic” /replace = “a” variation 7066 /note = “SNP; nongenic” /replace = “t”
variation 7086 /note = “SNP; nongenic” /replace = “t” enhancer 7187 . . . 7194 enhancer 7228 . . . 7235
enhancer 7387 . . . 7393 variation 7414 /note = “SNP; genic” /replace = “t” variation 7627
/note = “SNP; nongenic” /replace = “a” variation 7628 /note = “SNP; nongenic” /replace = “a” enhancer
7735 . . . 7742 variation 7792 /note = “SNP; nongenic” /replace = “a” enhancer
7965 . . . 7972 variation
8426 /note = “SNP; nongenic” /replace = “t” variation 8685 /note = “SNP; nongenic” /replace = “g”
variation 8693 /note = “SNP; nongenic” /replace = “a” variation 8790 /note = “SNP; nongenic”
/replace = “a” variation 8850 /note = “SNP; nongenic” /replace = “c” variation 8955 /note = “SNP;
nongenic” /replace = “t” variation 9024 /note = “SNP; nongenic” /replace = “t” variation 9079
/note = “SNP; nongenic” /replace = “a” variation 9167 /note = “SNP; nongenic” /replace = “c” enhancer
9168 . . . 9175 variation 9188 /note = “SNP; nongenic” /replace = “g” variation 9373 /note = “SNP;
nongenic” /replace = “a” variation 9430 /note = “SNP; nongenic” /replace = “g” variation 9453
/note = “SNP; nongenic” /replace = “a” enhancer 9566 . . . 9573 variation 9643 /note = “SNP; nongenic”
/replace = “t” variation 9763 /note = “SNP; nongenic” /replace = “a” variation 9807 /note = “SNP;
nongenic” /replace = “c” variation 9811 /note = “SNP; nongenic” /replace = “t” variation 9851
/note = “SNP; nongenic” /replace = “g” variation 9856 /note = “SNP; nongenic” /replace = “a” enhancer
9868 . . . 9874 variation 10297 /note = “SNP; nongenic” /replace = “a” variation 10404 /note = “SNP;
nongenic” /replace = “t” variation 10446 /note = “SNP; nongenic” /replace = “g” enhancer
10559 . . . 10565 variation 10756 /note = “SNP; nongenic” /replace = “c” variation 10841 /note = “SNP;
nongenic” /replace = “t” mRNA complement(join(<10862 . . . 10888, 11189 . . . 11737, 11826 . . . 11933,
12924 . . . 13451, 14301 . . . 14430, 14800 . . . >15479)) /product = “hypothetical protein” CDS
complement(join(10862 . . . 10888, 11189 . . . 11737, 11826 . . . 11933,
12924 . . . 13451, 14301 . . . 14430, 14800 . . . 15479))
/note = “region carrying Rhg1 gene; DUF399; Pfam 04187, domain of unknown function (DUF3411); Pfam
11891, pBlueScript II SK(+), EcoRI, protein of unknown function; conserved domain; similar to INSD
accession number AF526257” /codon_start = 1 /product = “hypothetical protein”
/protein_id = “AET79247.1” /db_xref = “GI: 357432833”
/translation = “MKPHTPASSFVTRLPHVPYFRGATAARAAPPDPPHDAPGGLEFR
RVSTAKRRRVSLSVCHASRVTAASNPGGSDGDGDTRARSSRRGVLMAPFLVAGASILL
SAATARAEEKAAESPLASAPKPEEPPKKKEEEEVITSRIYDATVIGEPLAIGKEKGKV
WEKLMNARVVYLGEAEQVPVRDDRELELEIVKNLHRRCLEKEKLLSLALEVFPANLQE
PLNQYMDKKIDGDTLKSYTLHWPPQRWQEYEPILSYCRENGIHLVACGTPLKILRTVQ
AEGIRGLTKDERKLYAPPAGSGFISGFTSISRRSSVDSTQNLSIPFGPSSYLSAQARV
VDEYSMSQIILQNVLDGGVTGMLIVVTGASHVTYGSRGTGVPARISGKIQKKNHAVIL
LDPERQFIRREGEVPVADFLWYSAARPCSRNCFDRAEIARVMNAAGRRRDALPQDLQK
GIDLGLVSPEVLQNFFDLEQYPLISELTHRFQGFRERLLADPKFLHRLAIEEAISITT
TLLAQYEKRKENFFQEIDYVITDTVRGSVVDFFTVWLPAPTLSFLSYADEMKAPDNIG
SLMGLLGSIPDNAFQKNPAGINWNLNHRIASVVFGGLKLASVGFISSIGAVASSNSLY
AIRKVLNPAVVTEQRIMRSPILKTAFIYACFLGISANLRYQAVFEVDGG” variation 11432 /note = “SNP;
genic” /replace = “a” variation 11648 /note = “SNP; genic” /replace = “g” variation 11772
/note = “SNP; nongenic” /replace = “g” variation 11868 /note = “SNP; genic” /replace = “c” variation
11948 /note = “SNP; nongenic” /replace = “a” variation 12025 /note = “SNP; nongenic” /replace = “a”
variation 12029 /note = “SNP; nongenic” /replace = “g” variation 12114 /note = “SNP; nongenic”
/replace = “a” variation 12115 /note = “SNP; nongenic” /replace = “a” variation 12153 /note = “SNP;
nongenic” /replace = “g” variation 12625 /note = “SNP; nongenic” /replace = “g” variation 12870
/note = “SNP; nongenic” /replace = “c” enhancer 12937 . . . 12949 enhancer 13095 . . . 13101 enhancer
13924 . . . 13930 variation 14153 /note = “SNP; nongenic” /replace = “a” enhancer 14388 . . . 14395
variation 14628 /note = “SNP; nongenic” /replace = “t” enhancer 15045 . . . 15052 enhancer
15076 . . . 15083 enhancer 15095 . . . 15111 enhancer
15157 . . . 15169 enhancer 15187 . . . 15194 enhancer
15272 . . . 15285 variation 15345 /note = “SNP;
genic” /replace = “g” enhancer 15387 . . . 15394 variation
15389 /note = “SNP; genic” /replace = “a” enhancer 15434 . . . 15446 promoter
complement(15812 . . . 15862) variation 15895 /note = “SNP;
nongenic” /replace = “g” variation 16069
/note = “SNP; nongenic” /replace = “t” variation 16189 /note = “SNP; nongenic” /replace = “a” variation
16823 /note = “SNP; nongenic” /replace = “c” variation 16876 /note = “SNP; nongenic” /replace = “t”
variation 17247 /note = “SNP; nongenic” /replace = “g” enhancer 17346 . . . 17354 variation 17660
/note = “SNP; nongenic” /replace = “g” variation 17743 /note = “SNP; nongenic” /replace = “a” variation
17778 /note = “SNP; nongenic” /replace = “a” variation 18131 /note = “SNP; nongenic” /replace = “c”
mRNA complement(join(<18721 . . . 18782, 20280 . . . 20325, 20407 . . . 20475,
21909 . . . 21971, 22076 . . . 22336, 22869 . . . 22922, 23180 . . . 23256,
23795 . . . >23999)) /product = “hypothetical protein”
CDS complement(join(18721 . . . 18782, 20280 . . . 20325, 20407 . . . 20475,
21909 . . . 21971, 22076 . . . 22336, 22869 . . . 22922,
23180 . . . 23256, 23795 . . . 23999)) /note = “conserved domain,
cd06850; biotinyl-domain or biotin carboxyl carrier protein (BCCP) domain is present in all biotin-
dependent enzymes such as acetyl-CoA carboxylase, pyruvate carboxylase, propionyl-CoA carboxylase,
methylcrotonyl-CoA carboxylase, geranyl-CoA carboxylase; similar to INSD accession number ACU19037”
/codon_start = 1 /product = “hypothetical protein”
/protein_id = “AET79248.1” /db_xref = “GI: 357432834”
/translation = “MGTMSHVRACLEKQAVLPIHNARWNSKRRLFIQHLAYGQKHINS
HMKGKSTLVSSAKTAEAINTSNSDASSDNTPQGSLEKKPLQTATFPNGFEALVLEVCD
ETEIAELKVKVGDFEMHIKRNIGATKVPLSNISPTTPPPIPSKPMDESAPNSLPPSPP
KSSPEKNNPFANVSKEKSPKLAALEASGTNTYVLVTSPTVGLFRRGRTVKGKKQPPIC
KEGDVIKEGQVIGYLDQFGTGLPIRSDVAGEVLKLLVEDGEPVGYGDRLIAVLPSFHD IK” variation
19479 /note = “SNP; nongenic” /replace = “t” variation 19621 /note = “SNP; nongenic” /replace = “g”
enhancer 19672 . . . 19679 enhancer 19777 . . . 19783 variation 20724 /note = “SNP; nongenic”
/replace = “t” enhancer 20978 . . . 20985
enhancer 21015 . . . 21022 variation 21349 /note = “SNP; nongenic”
/replace = “c” variation 22265 /note = “SNP; genic” /replace = “c” variation 23317 /note = “SNP;
nongenic” /replace = “a” variation 23697 /note = “SNP; nongenic” /replace = “t” enhancer
23854 . . . 23861 promoter complement(24024 . . . 24074) variation 24181 /note = “SNP; nongenic”
/replace = “c” variation 24184 /note = “SNP; nongenic” /replace = “g” variation 24338 /note = “SNP;
nongenic” /replace = “a” enhancer 24814 . . . 24821
enhancer 24852 . . . 24858 enhancer 25010 . . . 25017
variation 25204 /note = “SNP;
nongenic” /replace = “a” enhancer 25320 . . . 25327 variation 25478
/note = “SNP; nongenic” /replace = “t”
enhancer 25613 . . . 25619 variation 25677 /note = “SNP; nongenic”
/replace = “c” variation 25730 /note = “SNP; nongenic” /replace = “g” variation 25802 /note = “SNP;
nongenic” /replace = “t” variation 26700 /note = “SNP; nongenic” /replace = “a” variation 26776
/note = “SNP; nongenic” /replace = “a” variation
26967 /note = “SNP; nongenic” /replace = “t” variation
26968 /note = “SNP; nongenic” /replace = “t” variation 27115 /note = “SNP; nongenic” /replace = “a”
variation 27328 /note = “SNP; nongenic” /replace = “c” variation 27409 /note = “SNP; nongenic”
/replace = “c” repeat_region 27441 . . . 27451
/rpt_type = tandem /satellite = “microsatellite:SIUCSat1”
variation 27551 /note = “SNP; nongenic” /replace = “t” mRNA
complement(join(<28033 . . . 28429, 29318 . . . 29496, 29597 . . . 29677,
29785 . . . 29892, 30076 . . . >30207))
/product = “hypothetical protein” CDS
complement(join(28033 . . . 28429, 29318 . . . 29496, 29597 . . . 29677,
29785 . . . 29892, 30076 . . . 30207)) /note = “region carrying Rhg1
gene, pBlueScript II SK(+), EcoRI; similar to
INSD accession number AF526260” /codon_start = 1 /product = “hypothetical protein”
/protein_id = “AET79249.1” /db_xref = “GI: 357432835”
/translation = “MANNFLDVFCWIQNLPPISEWETSSMSLNICSSSSSCQPRLNLT
AILLYGSNKNSTTFIRFPNLDSTASDNLSDVFNLSDSRQASHMIMKLLGSNLEELWMR
SLNLAVQLYCHLMVMDVENSKSSPASERLQFSLRYHHVEGVLQFNHKVLIKDEWAEIM
VDIDNVRCDVIELVNEFLMKQRGAGAAEKHFPSRISLQLTPTIQDQVLSLSVGKSSEN
PRKEIGVDKSVEASFEASNPLALKVSAGESPQPLVYGYSANLNWFLHDCVDGKEVLSS
KPSKFAMLNPKSWFKNRYSSAY” variation 28131 /note = “SNP; genic” /replace = “t” enhancer
29433 . . . 29440 enhancer 29537 . . . 29543 enhancer 29987 . . . 29993 promoter
complement(30433 . . . 30483)
enhancer 30517 . . . 30524 enhancer 31096 . . . 31102 enhancer 31519 . . . 31526 variation 31536
/note = “SNP; nongenic” /replace = “g” variation 31669 /note = “SNP; nongenic” /replace = “c” enhancer
31821 . . . 31828 variation 31822 /note = “SNP; nongenic” /replace = “g” variation 31834 /note = “SNP;
nongenic” /replace = “a” variation 31910 /note = “SNP; nongenic” /replace = “g” variation 31943
/note = “SNP; nongenic” /replace = “a” variation 31982 /note = “SNP; nongenic” /replace = “a” variation
32054 /note = “SNP; nongenic” /replace = “a” variation 32056 /note = “SNP; nongenic” /replace = “c”
variation 32076 /note = “SNP; nongenic” /replace = “c” enhancer 32113 . . . 32119 variation 32191
/note = “SNP; nongenic” /replace = “g” variation 32210 /note = “SNP; nongenic” /replace = “t” variation
32248 /note = “SNP; nongenic” /replace = “c” variation 32501 /note = “SNP; nongenic” /replace = “c”
variation 32547 /note = “SNP; nongenic” /replace = “a” variation 32549 /note = “SNP; nongenic”
/replace = “t” variation 32590 /note = “SNP; nongenic” /replace = “t” variation 32592
/note = “SNP;
nongenic” /replace = “t” variation 32607 /note = “SNP; nongenic” /replace = “t” variation 32623
/note = “SNP; nongenic” /replace = “g” variation 32674 /note = “SNP; nongenic” /replace = “t” variation
32699 /note = “SNP; nongenic” /replace = “g” variation 32730 /note = “SNP; nongenic” /replace = “c”
variation 32748 /note = “SNP; nongenic” /replace = “c” variation 32776 /note = “SNP; nongenic”
/replace = “t” variation 32779 /note = “SNP;
nongenic” /replace = “t” variation 32783 /note = “SNP;
nongenic” /replace = “a” variation 32788 /note = “SNP; nongenic” /replace = “g” variation 32816
/note = “SNP; nongenic” /replace = “c” variation 32819 /note = “SNP; nongenic” /replace = “a” variation
32830 /note = “SNP; nongenic” /replace = “g” variation 32831 /note = “SNP; nongenic” /replace = “g”
variation 32833 /note = “SNP; nongenic” /replace = “g” variation 32842 /note = “SNP; nongenic”
/replace = “c” variation 32871 /note = “SNP; nongenic” /replace = “a” variation 32875 /note = “SNP;
nongenic” /replace = “g” variation 32891 /note = “SNP; nongenic” /replace = “g” variation 32936
/note = “SNP; nongenic” /replace = “c” variation 32955 /note = “SNP; nongenic” /replace = “c” variation
32962 /note = “SNP; nongenic” /replace = “t” variation 32974 /note = “SNP; nongenic” /replace = “g”
variation 32978 /note = “SNP; nongenic” /replace = “c” variation 32997 /note = “SNP; nongenic”
/replace = “a” variation 33021 /note = “SNP; nongenic” /replace = “g” variation 33027 /note = “SNP;
nongenic” /replace = “t” variation 33044 /note = “SNP; nongenic” /replace = “a” variation 33091
/note = “SNP; nongenic” /replace = “t” variation 33098 /note = “SNP; nongenic” /replace = “t” variation
33101 /note = “SNP; nongenic” /replace = “t” variation 33189 /note = “SNP; nongenic” /replace = “a”
variation 33205 /note = “SNP; nongenic” /replace = “c” variation 33363 /note = “SNP; nongenic”
/replace = “g” variation 33548 /note = “SNP; nongenic” /replace = “g” variation 33858 /note = “SNP;
nongenic” /replace = “a” repeat_region 33889 . . . 33894 /rpt_type = tandem
/satellite = “microsatellite:SIUCSac13” variation 33997 /note = “SNP; nongenic” /replace = “t” enhancer
34224 . . . 34231 variation 34240 /note = “SNP; nongenic” /replace = “t” variation 34249 /note = “SNP;
nongenic” /replace = “a” variation 34440 /note = “SNP; nongenic” /replace = “a” enhancer
34469 . . . 34476 variation 34512 /note = “SNP; nongenic” /replace = “g” variation 34563 /note = “SNP;
nongenic” /replace = “a” variation 34654 /note = “SNP; nongenic” /replace = “g” variation 34965
/note = “SNP; nongenic” /replace = “g” variation 35056 /note = “SNP; nongenic” /replace = “a” variation
35072 /note = “SNP; nongenic” /replace = “t” variation 35095 /note = “SNP; nongenic” /replace = “a”
variation 35567 /note = “SNP; nongenic” /replace = “a” variation 35700 /note = “SNP; nongenic”
/replace = “c” variation 35764 /note = “SNP; nongenic” /replace = “a” variation
complement(35785 . . . 37785) /note = “SNP; genic” /replace = “a” enhancer 35967 . . . 35975 gene
36069 . . . >39204 /gene = “rhg1g” promoter 36069 . . . 36119 /gene = “rhg1g” mRNA
join(<36448 . . . 38413, 38606 . . . >39204)
/gene = “rhg1g” /product = “receptor-like protein kinase” CDS
join(36448 . . . 38413, 38606 . . . 39204) /gene = “rhg1g” /inference = “similar to DNA
sequence: INSD: AF506516.1” /note = “conserved domain, protein kinases (PKs), catalytic (c) domain,
cd00180, PKc; similar to INSD accession number AB495276” /codon_start = 1 /product = “receptor-like
protein kinase” /protein_id = “AET79243.1” /db_xref = “GI: 357432829”
/translation = “MVVAVEKTNLTSQSQCFNRVSDKKKERCKTHMNNVNPCCFLFLL
CVWSLVVLPSCVRPVLCEDEGWDGVVVTASNLLALEAFKQELADPEGFLRSWNDSGYG
ACSGGWVGIKCAQGQVIVIQLPWKGLRGRITDKIGQLQGLRKLSLHDNQIGGSIPSTL
GLLPNLRGVQLFNNRLTGSIPLSLGFCPLLQSLDLSNNLLTGAIPYSLANSTKLYWLN
LSFNSFSGPLPASLTHSFSLTFLSLQNNNLSGSLPNSWGGNSKNGFFRLQNLILDHNF
FTGDVPASLGSLRELNEISLSHNKFSGAIPNEIGTLSRLKTLDISNNALNGNLPATLS
NLSSLTLLNAENNLLDNQIPQSLGRLRNLSVLILSRNQFSGHIPSSIANISSLRQLDL
SLNNFSGEIPVSFDSQRSLNLFNVSYNSLSGSVPPLLAKKFNSSSFVGNIQLCGYSPS
TPCLSQAPSQGVIAPPPEVSKHHHHRKLSTKDIILIVAGVLLVVLIILCCVLLFCLIR
KRSTSKAGNGQATEGRAATMRTEKGVPPVAGGDVEAGGEAGGKLVHFDGPMAFTADDL
LCATAEIMGKSTYGTVYKAILEDGSQVAVKRLREKITKGHREFESEVSVLGKIRHPNV
LALRAYYLGPKGEKLLVFDYMSKGSLASFLHGGGTETFIDWPTRMKIAQDLARGLFCL
HSQENIIHGNLTSSNVLLDENTNAKIADFGLSRLMSTAANSNVIATAGALGYRAPELS
KLKKANTKTDIYSLGVILLELLTRKSPGVSMNGLDLPQWVASVVKEEWTNEVFDADLM
RDASTVGDELLNTLKLALHCVDPSPSARPEVHQVLQQLEEIRPERSVTASPGDDIV” variation 36707
/gene = “rhg1g” /note = “SNP; genic” /replace = “t” variation 36790 /gene = “rhg1g” /note = “SNP;
genic” /replace = “a” variation 36858 /gene = “rhg1g” /note = “SNP; genic” /replace = “t” variation
37446 /gene = “rhg1g” /note = “SNP; genic” /replace = “a” variation 37506 /gene = “rhg1g”
/note = “SNP; genic” /replace = “t” variation 37587
/gene = “rhg1g” /note = “SNP; genic” /replace = “t”
enhancer 37917 . . . 37924 /gene = “rhg1g” variation 38331 /gene = “rhg1g” /note = “SNP; genic”
/replace = “a” variation 38643 /gene = “rhg1g” /note = “SNP; genic” /replace = “a” enhancer
39052 . . . 39058 /gene = “rhg1g” enhancer 39480 . . . 39487 enhancer 39703 . . . 39710 variation 39882
/note = “SNP; nongenic” /replace = “t” variation 39992 /note = “SNP; nongenic” /replace = “a” variation
40024 /note = “SNP; nongenic” /replace = “t” variation 40074 /note = “SNP; nongenic” /replace = “c”
variation 40218 /note = “SNP; nongenic” /replace = “t” variation 40260 /note = “SNP; nongenic”
/replace = “g” variation 40827 /note = “SNP; nongenic” /replace = “a” variation 41160 /note = “SNP;
nongenic” /replace = “c” variation 42110 /note = “SNP; nongenic” /replace = “g” enhancer
42621 . . . 42628 enhancer 42935 . . . 42943 variation 45256 /note = “SNP; nongenic” /replace = “c”
variation 45261 /note = “SNP; nongenic” /replace = “g” variation 45268 /note = “SNP; nongenic”
/replace = “c” enhancer 46062 . . . 46069
enhancer 46578 . . . 46585 variation 46617 /note = “SNP; nongenic”
/replace = “a” enhancer 47217 . . . 47224
variation 47400 /note = “SNP; nongenic” /replace = “c” variation
47820 /note = “SNP; nongenic” /replace = “t” gene 47832 . . . >48345 /gene = “LOC547704” promoter
47832 . . . 47882 /gene = “LOC547704” mRNA
join(<47930 . . . 48055, 48169 . . . >48345) /gene = “LOC547704”
/product = “diphenol oxidase laccase” CDS
join(47930 . . . 48055, 48169 . . . 48345) /gene = “LOC547704”
/note = “TIGR03389, similar to INSD accession number AF527604” /codon_start = 1 /product = “diphenol
oxidase laccase” /protein_id = “AET79244.1” /db_xref = “GI: 357432830”
/translation = “MEPAKTIHNNVKYSPIFLAIFVLILASALSSANAKIHEHEFVVE
ATPVKRLCKTHNSITVNGQYPGPTLEINNGDTLVVKVTNKARYNVTIHWYNIKLAS” gene
48767 . . . >52465 /gene = “Glyma18g02690.1” promoter 48767 . . . 48817 /gene = “Glyma18g02690.1”
mRNA join(<48837 . . . 48853, 48919 . . . 49163,
49420 . . . 49548, 50443 . . . 50984, 51537 . . . 51981, 52335 . . . >52465)
/gene = “Glyma18g02690.1” /product = “multicopper oxidase” CDS
join(48837 . . . 48853, 48919 . . . 49163,
49420 . . . 49548, 50443 . . . 50984, 51537 . . . 51981, 52335 . . . 52465)
/gene = “Glyma18g02690.1” /note = “laccase” /codon_start = 1 /product = “multicopper oxidase”
/protein_id = “AET79245.1” /db_xref = “GI: 357432831”
/translation = “MAFFSGHGVRQMRTGWADGPEFVTQCPIRPGGSYTYRFTVQGQE
GTLWWHAHSSWLRATVYGALIIRPREGEPYPFPKPKHETPILLGEWWDANPIDVVRQA
TRTGGAPNVSDAYTINGQPGDLYKCSSKDTTIVPIHAGETNLLRVINAALNQPLFFTV
ANHKLTVVGADASYLKPFTTKVLILGPGQTTDVLITGDQPPSRYYMAARAYQSAQNAA
FDNTTTTAILEYKSPNHHNKHSHHRAKGVKNKTKPIMPPLPAYNDTNAVTSFSKSFRS
PRKVEVPTEIDQSLFFTVGLGIKKCPKNFGPKRCQGPINGTRFTASMNNVSFVLPNNV
SILQAHHLGIPGVFTTDFPGKPPVKFDYTGNVSRSLWQPVPGTKAHKLKFGSRVQIVL
QDTSIVTPENHPIHLHGYDFYIVAEGFGNFDPKKDTAKFNLVDPPLRNTVAVPVNGWA
VIRFVADNPGAWLLHCHLDVHIGWGLATVLLVENGVGKLQSIEPPPVDLPLC” variation 48870
/gene = “Glyma18g02690.1” /note = “SNP; nongenic” /replace = “a” enhancer 48992 . . . 48999
/gene = “Glyma18g02690.1” variation 49749 /gene = “Glyma18g02690.1” /note = “SNP; nongenic”
/replace = “g” variation 49894 /gene = “Glyma18g02690.1” /note = “SNP; nongenic” /replace = “a”
variation 49920 /gene = “Glyma18g02690.1” /note = “SNP; nongenic” /replace = “a” variation 50233
/gene = “Glyma18g02690.1” /note = “SNP; nongenic” /replace = “g” variation 50328
/gene = “Glyma18g02690.1” /note = “SNP; nongenic” /replace = “t” variation 50418
/gene = “Glyma18g02690.1” /note = “SNP; nongenic” /replace = “g” variation 50603
/gene = “Glyma18g02690.1” /note = “SNP; genic” /replace = “g” enhancer 50676 . . . 50682
/gene = “Glyma18g02690.1” variation 50720 /gene = “Glyma18g02690.1” /note = “SNP; genic”
/replace = “c” variation 50779 /gene = “Glyma18g02690.1” /note = “SNP; genic” /replace = “a” variation
50856 /gene = “Glyma18g02690.1” /note = “SNP; genic” /replace = “g” enhancer 50933 . . . 50940
/gene = “Glyma18g02690.1” enhancer 51035 . . . 51042 /gene = “Glyma18g02690.1” variation 51370
/gene = “Glyma18g02690.1” /note = “SNP; nongenic” /replace = “t” variation 52118
/gene = “Glyma18g02690.1” /note = “SNP; nongenic” /replace = “t” variation 52131
/gene = “Glyma18g02690.1” /note = “SNP; nongenic” /replace = “a” enhancer 52336 . . . 52343
/gene = “Glyma18g02690.1” enhancer 52662 . . . 52668 variation 52691 /note = “SNP; nongenic”
/replace = “c” variation 52934 /note = “SNP;
nongenic” /replace = “c” enhancer 52983 . . . 52989 variation
53612 /note = “SNP; nongenic” /replace = “g” variation 54135 /note = “SNP; nongenic” /replace = “t”
variation 54461 /note = “SNP; nongenic” /replace = “a” variation 54558 /note = “SNP; nongenic”
/replace = “a” gene complement(<54579 . . . >57894) /gene = “Glyma18g02700.1” mRNA
complement(join(<54579 . . . 55772, 56501 . . . 57364,
57661 . . . >57894)) /gene = “Glyma18g02700.1”
/product = “putative K+/H+-antiporter” CDS complement(join(54579 . . . 55772,
56501 . . . 57364, 57661 . . . 57894))
/gene = “Glyma18g02700.1” /codon_start = 1 /product = “putative K+/H+-antiporter”
/protein_id = “AET79250.1” /db_xref = “GI: 357432836”
/translation = “MSESSITTVFNDARTGQMIVCLKNDRTVGSLGVWMGDNPFDFVV
PVTLFQIILVSLLSKALHYVLRPINTPKFICCVIAGILLGPTFLGRHEEILGALFPVR
QSLFLNTLSKIGTTYCVFLTCLKMDVVTTLKSAKRCWRFGVFPFLASFLVTVTLFSLY
SPNGNANQNQMSIYHFPNIFTLSSFAVVSETLMELNLVATELGQIALSSAMISEILQW
TTMELLFNSKFSMRFLIVLLIGATGFAVLLLLIIRPLVNIVLERTPPGKPIKEAYVVL
LLLGPLVMAAISDTFGIYFVMGPFLYGLVLPNGPPLATTIIERSELIVYEFFMPFFFL
LIGTRTDLTLIHEHWEVVLVVLAILFVGCLVKVIDTEVFSVAVMSVVVMTSICIPLIK
SLYRHRRVCKTQTIQEGSVKTIQNITENTPFNIVSCVHTDEHVHNMIALIEACNPTTQ
SPLYVYVVHLIELVGKSTPILLPMNKNKRKSLSVNYPNTNHILRAFENYSNNSSGPVT
VLSYVNVAPYRSMHEAVCNLAEDNSVHLLIIPFHQNDQTLGSHLASTIRNLNTNFLAN
AKGTLGILVDRYSVLSGSSSKLSFDVGIFFIGGKDDREALALGIRMLERPNTRVTLFR
FVLPTNEDSRFNGLVENEDENLESTLDESLIDEFIAKNDISSDSVNVVYHEAVVEDCI
QVLKAIRGMEKDYDLVMVGKRHSMGNFVEEEMSNFMDNADQLGILGDMLASNEFCNGK
VPVLVMQCGDEKRVKQLEKVCHI” variation 54654 /gene = “Glyma18g02700.1” /note = “SNP; genic”
/replace = “c” enhancer 55011 . . . 55017
variation 55643 /gene = “Glyma18g02700.1” /note = “SNP; genic”
/replace = “a” variation 55856 /gene = “Glyma18g02700.1” /note = “SNP; nongenic” /replace = “g”
variation 55972 /gene = “Glyma18g02700.1” /note = “SNP; nongenic” /replace = “t” variation 56274
/gene = “Glyma18g02700.1” /note = “SNP; nongenic” /replace = “t” variation 56444
/gene = “Glyma18g02700.1” /note = “SNP; nongenic” /replace = “g” variation 56513
/gene = “Glyma18g02700.1” /note = “SNP; genic” /replace = “t” variation 56726
/gene = “Glyma18g02700.1” /note = “SNP; genic” /replace = “g” variation 57554
/gene = “Glyma18g02700.1” /note = “SNP; nongenic” /replace = “t” variation 57999 /note = “SNP;
nongenic” /replace = “g” promoter complement(58008 . . . 58058) variation 58074 /note = “SNP;
nongenic” /replace = “a” variation 58075 /note = “SNP; nongenic” /replace = “t” variation 58162
/note = “SNP; nongenic” /replace = “t” variation 58279 /note = “SNP; nongenic” /replace = “a” variation
58349 /note = “SNP; nongenic” /replace = “a” repeat_region 58708 . . . 58724 /rpt_type = tandem
/satellite = “microsatellite:TMD1” variation 58718
/note = “SNP; nongenic” /replace = “t” variation 58720
/note = “SNP; nongenic” /replace = “c” variation 58722 /note = “SNP; nongenic” /replace = “t” variation
58728 /note = “SNP; nongenic” /replace = “t” variation 58774 /note = “SNP; nongenic” /replace = “g”
variation 58862 /note = “SNP; nongenic” /replace = “g” variation 59074 /note = “SNP; nongenic”
/replace = “a” variation 59282 /note = “SNP; nongenic” /replace = “t” variation 59369 /note = “SNP;
nongenic” /replace = “t” variation 59494 /note = “SNP; nongenic” /replace = “g” variation 59516
/note = “SNP; nongenic” /replace = “c” gene complement(<59654 . . . >64305) /gene = “CHX23” mRNA
complement(join(<59654 . . . 60904, 61312 . . . 62310,
63227 . . . 63414, 64066 . . . 64102, 64165 . . . >64305))
/gene = “CHX23” /product = “monovalent cation H+ exchanger 23” CDS
complement(join(59654 . . . 60904, 61312 . . . 62310,
63227 . . . 63414, 64066 . . . 64102, 64165 . . . 64305))
/gene = “CHX23” /note = “cation:proton antiporter; sodium:hydrogen antiporter; conserved domain,
PLN03159; similar to INSD accession number AT1G05580; F3F20.2; F3F20_2” /codon_start = 1
/product = “monovalent cation H+ exchanger 23” /protein_id = “AET79251.1” /db_xref =
“GI: 357432837”
/translation = “MATSRGNGIVSSYWDSHGQWQVCVEDDRNVGSLGIFIGDRPFEF
VLPASKNTQIHLQRPVSPLENSRKRHKLDRCTSPTRQCDNVVYSSKLKVVEAVPNYSS
KSPMLDSTSPIVVEECLRYGGGIILGPTFLGRNKTYWQVLFPPRQTEYLVMASLTGAV
YFVFLVALKMDVLMTIRAAKSTWRLGVIPFLASFVVILALLCLYYHPQQISSASLTIA
RVSVSCLMSLSNFPVVSDAMLELNLTATELGQIALSSSMINDIILWLFIVMHSFTSNV
DVKKSIALLGNWCLLVFFNFFVLRPTMKLIAMRTPVGKPVKELYVVLILLGVLVMAGV
GDLMGVTFLMGPLIFGLVVPSGPPLGTTLAEKSEVLTTEFLLPFFFVYIGINTDLSAL
EDWRLFLTLQGVFFAGDLAKLLACVLVSLAYNIRPKHGTLLGLMLNIKGITQLISLAR
FKKQKMLDEDTFSQLVFCVVLITAIVTPLVNILYKHRPRVHAESLFEGELRTIQSTPR
NREFHIVCCVHNEANVRGITALLEECNPVQESPICVYAVHLIELVGKSAPILLPIKHR
HGRRKFLSVNYPNTNHIMQAFENYSNNSSGPVKVLPYINVAPYKSMHDAIFNLAQDNM
VPFIIIPFHENGNIDLVGHVAASIRKMNTRFQAHAPCTLGILVDRHSRLGASNNNNMY
FNVGVFFIGGAHDREALALGIRMSERADTRVSLFRFVIVNKKPCGCKIILTREEREEE
EEDTMLDEGLIDEFKSMKYGIGNVCWYEITVDDGVEVLEAVHSLEGNYDLVMVGRRHN
DGSLNGKEMTTFMENADALGILGDMLSSVEFCMGMVPVLVTQCGGVKISSSSNNKLDR
VGSVNVSQKRLSVHK” enhancer 60284 . . . 60291 variation 60335 /gene = “CHX23” /note = “SNP;
genic” /replace = “t” enhancer 60336 . . . 60344
enhancer 60480 . . . 60487 enhancer 60848 . . . 60854 variation
60956 /gene = “CHX23” /note = “SNP; nongenic” /replace = “t” variation 61126 /gene = “CHX23”
/note = “SNP; nongenic” /replace = “t” variation 61185 /gene = “CHX23” /note = “SNP; nongenic”
/replace = “g” variation 62327 /gene = “CHX23” /note = “SNP; nongenic” /replace = “g” variation
62358 /gene = “CHX23” /note = “SNP; nongenic” /replace = “a” variation 62686 /gene = “CHX23”
/note = “SNP; nongenic” /replace = “g” gene 64551 . . . >73760
/gene = “CHR38” promoter 64551 . . . 64601
/gene = “CHR38” promoter complement(64814 . . . 64864) variation 64926 /gene = “CHR38” /note = “SNP;
nongenic” /replace = “c” mRNA join(<64996 . . . 65077,
69033 . . . 69280, 69912 . . . 71246, 71554 . . . 71700,
71835 . . . >73760) /gene = “CHR38” /product = “chromatin remodeling 38” CDS
join(64996 . . . 65077, 69033 . . . 69280, 69912 . . . 71246,
71554 . . . 71700, 71835 . . . 73760) /gene = “CHR38”
/note = “nucleic acid-binding protein; CLASSY1; CLSY; FUNCTIONS IN: helicase activity, DNA-binding,
ATP-binding, nucleic acid-binding; INVOLVED IN: gene silencing by RNA; LOCATED IN: nucleolus;
EXPRESSED IN: 13 plant structures; EXPRESSED DURING: 6 growth stages; CONTAINS InterPro
DOMAIN: DEAD-like helicase, N-terminal (InterPro: IPR014001), DNA RNA helicase, C-terminal
(InterPro: IPR001650), helicase, superfamily 1 and 2, ATP-binding (InterPro: IPR014021), SNF2-related
(InterPro: IPR000330); BEST Arabidopsis thaliana protein match is: CHR42 (chromatin remodeling 42);
ATP-binding DNA-binding helicase nucleic acid-binding; similar to INSD accession number AT3G42670”
/codon_start = 1 /product = “chromatin remodeling 38” /protein_id = “AET79246.1”
/db_xref = “GI: 357432832”
/translation = “MEASVLLGNSYGVVARVAVAGLRGGVMAFEAYLSRSWRAVELIK
FESGTTTLYFVDNHHMTIKKGSFSDVRVRSRKATLSDCSFLRTGIDICVLSASQGNDN
SDESSANHVWLDAKINSIQRKPHNPECSCQYYVNFYVNQGSLGTELRTLRKEVKVVGI
NEIAILQKLERNTCQHKYYRWESSEDCSKVPHTKLLGKFISDLSWLVVASAIRKVSFC
ARSVENNIVYQILGSDATTSSLYMDSEISVVNFKVNEDGMQMPVIHLVDLFETDTNTS
GDKHDSHYDEVPSSYGFEGLRRSKRRNIQPERYSDCGNVSEIKVGNVRTWPYKLNKRK
DDDGGGEESLPLAQENSDNSQKVNELSSCREIIVYHGRNETLELKSGEANQTQLASVP
LLQEGDSLALEHHHLNDNVTRRSDAYYSTPKLKRKRLVDLEADVDFDPGREGINSNKG
VSEKRHGSSWYSRSRSHAAEHSYKDRSLNATAYKEMIDSYLKDVNRTPTTEEPPVMDQ
RKEIGNFGQKKEAEIPEREDEEQISEIDMLWREMEMALASSYLEETEGSNSANFAKTT
EESNRTCPHDYRLSEEIGIYCYKCGFVKTEIKYITPPFIEMQRSVRHQEEKQCNGKDT
KEKASKDDDFHLLSTHAPTDEHNSMEHDNVWKLIPQFREKLHDHQKKAFEFLWQNIGG
SMEPKLMDAESKRRGGCVISHAPGAGKTFLIIAFLVSYLKLFPGKKPLILAPKGTLYT
WCKEFNKWEISMPVYLIHGRGGTQKDTEQNSIVLPGFPNPNKYVKHVLDCLQKIKLWQ
EKPSVLVMSYTAFLALMREGSEFAHRKYMAKALREGPGILILDEGHNPRSTKSRLRKG
LMKLKTDLRILLSGTLFQNNFCEYFNTLCLARPKFISEVLDTLDPITRRKSKTVEKAG
HLLESRARKLFLDKIAKKIDSGIGNERMQGLNMLRETTNGFVDVYESENFDSAPGLQI
YTLLMNTTDKQREILPKLHTRVDECNGYPLELELLVTLGSIHPWLVKTTSCANKFFTA
DQLKQLDKYKYDMKAGSKVKFVLSLVFRVMQREKVLIFCHNLAPVKLLIELFEMFFKW
KKDREILLLSGELDLFERGKVIDKFEEHGGASKVLLASITACAEGISLTAASRVIFLD
SEWNPSKTKQAIARAFRPGQEKMVYVYQLLVTGTLEEDKYKRTTWKEWVSSMIFSEAF
EENLSHSRAVNIEDDILREMVEEDKSKTIHMILKNEKASTN” variation 66767 /gene = “CHR38”
/note = “SNP; nongenic” /replace = “t” variation 72575 /gene = “CHR38” /note = “SNP; genic”
/replace = “a” enhancer 73371 . . . 73378
/gene = “CHR38” variation 75037 /note = “SNP; nongenic”
/replace = “g” repeat_region 75777 . . . 75821
/rpt_type = tandem /satellite = “microsatellite:Satt 309”
repeat_region 75816 . . . 75860
/rpt_type = tandem /satellite = “microsatellite:SIUCSat27” variation 75926
/note = “SNP; nongenic” /replace = “t” variation 75965 /note = “SNP; nongenic” /replace = “c” variation
77617 /note = “SNP; nongenic” /replace = “a” variation 78547 /note = “SNP; nongenic” /replace = “t”
variation 78595 /note = “SNP; nongenic” /replace = “t” variation 79118 /note = “SNP; nongenic”
/replace = “a” variation 79411 /note = “SNP; nongenic” /replace = “t” variation 79434 /note = “SNP;
nongenic” /replace = “t” variation 79452 /note = “SNP; nongenic” /replace = “c” variation 79474
/note = “SNP; nongenic” /replace = “a” variation 79476 /note = “SNP; nongenic” /replace = “a” variation
79481 /note = “SNP; nongenic” /replace = “a” variation 79509 /note = “SNP; nongenic” /replace = “c”
variation 79655 /note = “SNP; nongenic” /replace = “t” enhancer 80759 . . . 80766

TABLE 2
B Markers and methods for allele discrimination at
Rhg1/Rfs2 by synonymous SNPs
I. Sequence Basis of markers

| | Haplotype | | | | | | |
|---|---|---|---|---|---|---|---|
| SNP | R1 | S1 | S6 | R2 | S3 | S4 | MR |
| Probe 560 | A | G | G | A | A | A | A |
| Probe 2090 | C | C | C | C | A | A | C |
| Probe 2007 | C | T | T | T | T | T | T |
| Probe 3229 | C | T | T | T | C | C | C |

II. Haplotypes distiguished

Probe 560 differentiates between (S1, S6) &
(R1, R2, S3, S4 AND MR)
Probe 2090 differentiates between (S3, S4) &
(R1, S1, S6, R2 AND MR)
Probe 2007 differentiates between (R1) and
(R2 & MR).
Probe 3229 differentiates between R2 and MR

*Using probes (560 and 2090) we can tell the
difference between resistance and susceptibility,
but not the type (ie. We can tell that a variety
is resistant (could be either R1, R2 or MR) or
susceptible (either of the S). Probe 2007
differentiates between (R1) and (MR, R2).
Probe 3229 differentiates between R2 and MR.

II. Haplotypes

| | | |
|---|---|---|
| R1-PEKING/FORREST/PI437.654 | HAP2 | R |
| S1-LEE | HAP7 | S |
| S6-NOIR | HAP6 | S |
| R2-PI88788 | HAP8 | R |
| S3-A3244 or PI567660 | HAP1 | S |
| S4-WILL | HAP4 | S |
| MR-TOYOSUZU | HAP3 | MR |
| S2-A2704 | HAP5 | S |
| S5-PI 490769 (Peking like) | HAP9 | R |

TABLE 2C
Primers for Taqman probes to synonymous
SNPS in the RLK

| Name | Primer Sequence | Tm ° C. |
|---|---|---|
| 560_1 F | CAGTGAAACTTGGAGATGCCAG | 57 |
| 560_1 R | TCAGTCTCAAATTAGTGAGGGAT | 54.7 |
| 560_1 Hex- | AAGTGTGATACACACTATCCCTCCTCCTCGT | 65.1 |
| 560_1 Fam- | AAGTGTGATACATACTATCCCTCCTCCTC | 65.3 |
| | GTGC | |
| 2090/2007_F | GGTTGTGACAGCATCAAACCTC | 57.4 |
| 2090/2007_R | CCT TCC AAG GAA GCT GGA TC | 56.2 |
| 2090_Fam | TAACCTGTCCCTGAGCACACTTGATTCC | 66.8 |
| | AACC | |
| 2090_Hex | CACAATAACCTGTCCCTTAGCACACTTGA | 66.6 |
| | TTCCAACC | |
| 2007_Fam | GCTTTCAAGCAAGAGTTGGCTGATCCAGAA | 66 |
| | GGGTTC | |
| 2007_Hex | AGCTTTCAAGCAAGAGTTGGTTGATCCA | 66 |
| | GAAGGGTTCT | |
| 3229_1F | CCATCATAGGAAGCTAAGCAC | 54 |
| 3229_1R | GCACACAAGAGATCATCAGC | 54.6 |
| 3229_1Hex | GGAGTTCTCCTCGTAGTCCTGATTATAC | 65 |
| | TTTGTTGTGTC C | |
| 3229_1Fam | GGAGTTCTCCTCGTAGTTCTGATTATACTT | 65 |
| | TGTTGTGTCC | |

TABLE 2D
Taqman markers for SNPs that alter amino
acid residues in the RLK

| | |
|---|---|
| Name | Primer Sequence |

Rhg1 RLK residue 87

| | |
|---|---|
| AHMSIJ4_F | CAGCATCAAACCTCTTAGCACTTG |
| AHMSIJ4_R | CATTCCAGCTCCGCAAGAAC |
| AHMSIJ4_V VIC | CTGGATCAGCCAACTC |
| AHMSIJ4_M FAM | TCTGGATCAACCAACTC |

Rhg1 RLK residue 71

| | |
|---|---|
| AHKAL7R_F | CCAGTTTTGTGTGAAGATGAAGGTT |
| AHKAL7R_R | GCTTCAAGTGCTAAGAGGTTTGATG |
| AHKAL7R_V VIC | CTGTCACAACCACTCC |
| AHKAL7R_M FAM | CTGTCACAGCCACTCC |

Rhg1 RLK residue 115

| | |
|---|---|
| AHLJKDZ_F | CGGAGGTTGGGTTGGAATCAA |
| AHLJKDZ_R | CCCTCAAACCCTTCCAAGGAA |
| AHLJKDZ_V VIC | CTGTCCCTGAGCACAC |
| AHLJKDZ_M FAM | CCTGTCCCTTAGCACAC |

Rhg1 RLK residue 274

| | |
|---|---|
| AHQJC2V_F | TGGGAATTCCAAGAATGGCTTCT |
| AHQJC2V_R | GCAGGAACGTCACCAGTGAAA |
| AHQJC2V_V VIC | TTGATCCTAGATCATAACTT |
| AHQJC2V_M FAM | TTTGATCCTAGATAATAACTT |

Rhg1 RLK residue 539

| | |
|---|---|
| AHT97LJ_F | CGGCCACTATGAGGACAGAAAA |
| AHT97LJ_R | CCTCCCCACCTGCTTCAAC |
| AHT97LJ_V VIC | CAGTTGCTGGTGGTGAT |
| AHT97LJ_M FAM | CAGTTGCTGTTGGTGAT |

Rhg1 RLK residue 770

| | |
|---|---|
| AH0IYGN_F | CTACAGTCTTGGTGTTATCTTGTTAGAACT |
| AH0IYGN_R | GCAACCCACTGAGGCAAATCTA |
| AH0IYGN_V VIC | CCATTCATAGACACCCC |
| AH0IYGN_M FAM | CCATTCATAGGCACCCC |

Supplementary Table 2E: The sequence of the markers and
primers that were developed from BAC73p06 sequences and
used for fine map development and marker assisted selection

| | | | | BAC73p06 (and AX19629) |
|---|---|---|---|---|
| Primer | | Sequence (5′-3′; R) | Tm (° C.) | coordinates |
| SIUC_Sat_-36 | F | TGTCCCTAAATAAATTAATAAATCCAA | 56 | 1,770 (11,870) |
| | R | AAATGACCCTCTCTCTCTCT | 48 | |
| Minisatt 1 | F | AGACAAAGCATTCTGATCGC | 59 | 33,900 (44,000) |
| (InD-3.5) | R | AGATTCGCCACTACTGTTGG | 60 | |
| SIUC_Satt3.5 | F | CCCAACATAATTCCAACTTCA | 58 | 40,150 (50,000) |
| | R | GCAACAATGCTAGCCATCAA | 59 | |
| SIUC-Saaag3.5 | F | TTCCAACTTCAAAATTCACTCAA | 57 | 40,200 (50,050) |
| | R | GTGTCATCAACAACGCAACA | 60 | |
| SIUC-Satt9.0 | F | GCACTAATGGTGACACACAC | 59 | 45,250 (54,750) |
| | R | TGAGTTAGACCTCCTCTCTTGT | 60 | |
| SIUC-Satt9.5 | F | CTTTAAAGTCTCAAGTCATTGGAT | 57 | 45,500 (55,000) |
| | R | CGAAAGGGCCTCTTTATGTT | 58 | |
| SIUC-Satt22 | F | CCTTGCAATAGAGGAGTACAA | 57 | 58,500 (58,500) |
| | R | AGTACGTGTCTTGATTTTATTTCTT | 57 | |
| SIUC Satt39 | F | GTTTTGGCAAGCAAGAGTCC | 60 | 75,800 (85,900) |
| | R | AACTAGTATGGTATAAAGTAAGGCT | 57 | |
| SIUC-Sat_40 | F | GCATGTTTGGCTCGCATTAAA | 60 | 79,500 (89,600) |
| | R | TCCCACTTCAATTAACTCATGC | 59 | |

### Example 5

### TaqMan™ Genotyping Assay

PCR primers and TaqMan™ probes were designed with the primer express program (Perkin-Elmer/Applied Biosystems, Foster City, Calif.) and were custom synthesized by Perkin-Elmer. The SNP genotyping assay within the gene encoding the RLK was performed using a custom Taqman Kit. Three probes were designed for the synonymous SNPs at 506 bp 2007 bp and 2090 bp (relative to the translation start site) to distinguish the 8 commonest alleles of the RLK (Table 2). Six probes were designed to distinguish the 8 commonest alleles of the RLK (Table 2). Probes 2090 bp and 115 amino acid were polymorphic in X5, Westag 97 and Essex compared to Forrest and were preferred here though all probes were useful for marker assisted selection during soybean breeding and for advancing transgenic lines in different genetic backgrounds. Such probes were used to detect gene expression from transgenes using reverse transcriptase and mRNA. Primer and probe optimizations used different combinations of each pair and optimizing to optimal signal strength and balanced fluorophore intensity.

TaqMan™ reactions were performed essentially as the Perkin-Elmer TaqMan™ PCR Reagent Kit protocol describes except the PCR reaction was performed in 384 well plates to reduce assay volume and cost. Briefly, each reaction contained 10 ng of the extracted DNA, 0.025 units/ml of AmpliTaq Gold™ (Perkin-Elmer/Applied Biosystems, Foster City, Calif.), 400 nM of the forward and reverse primers (Research Genetics, Huntsville, Ala.), 50 nM of FAM fluorescent probe and 150 nM of TET fluorescent probe (Perkin-Elmer/Applied Biosystems, Foster City, Calif.) in 1× universal master mix (Perkin-Elmer/Applied Biosystems, Foster City, Calif.). The above ratio of primers and probes was optimized using a series of primer/probe combinations to reach a maximal signal and the balance of the two probes by reading in an ABI 7200 sequence detector. The TaqMan™ universal PCR master mix is a premix of all the components, except primer and probes, necessary to perform a 5′ nuclease assay. The final optimized conditions represented a two step PCR protocol, with two holds followed by cycling, on a 384 well thermal cycler (GeneAmp PCR System 9700, Perkin-Elmer/Applied Biosystems, Foster City, Calif.). The two hold cycles were 50° C. for 2 min and 95° C. for 10 min. The 35 cycles were at 95° C. for 15 sec, 60° C. for 1 min. After amplification the plates were cooled to room temperature and samples were transferred from a 384 well plate to a 96 well MicroAmpJ optical tray and fluorescence was detected on an ABI PrismJ 7200 Sequence Detector (Perkin-Elmer/Applied Biosystems, Foster City, Calif.).

The results were analyzed by allelic discrimination of the sequence detection software (Perkin-Elmer/Applied Biosystems, Foster City, Calif.). Two grouping methods were used to attempt to accurately separate heterogeneous lines from homogeneous lines at each allele. In grouping method 1 (TaqMan™ 1) a stringent cut-off for FAM (>7) was used for allele 1 compared to heterogenous scores. This served to reduce the number called as potentially heterogeneous to about the percentage expected from the breeding method used for RIL development (6%). Fluorophore ratios were as follows; no amplification (FAM and TET both less than 6 units); allele 1 homozygous (FAM less than 7, TET greater than 7); allele 2 homozygous (FAM greater than 10, TET less than 5); and heterogeneous for allele 1 and allele 2 (FAM greater than 7, TET 5-8). For TaqMan™ selection grouping method 2 ratios were; no amplification (FAM and TET both less than 6 units); allele 1 homozygous (FAM less than 5, TET greater than 7); allele 2 homozygous (FAM greater than 10, TET less than 5); and heterogeneous for allele 1 and allele 2 (FAM greater than 5, TET 5-9). The FAM and TET signals were stable in the dark for 2 days after PCR.

### Example 6

### Genotyping Assay Using Gel Electrophoresis Markers

PCR reactions were performed with DNA from the recombinant inbred lines, Nils and transgenic plants. The marker TMD1 amplified a fragment from Rfs2/Rhg1 of 303+15 bp (resistant allele was the smaller) and of 362 bp from a syntenic homeolog of Rfs2/rhg1 found in the sequence of BAC H38F23 from Lg B1 (chromosome 11; SEQ ID No: 33). Presence of the Rhg1/Rfs2 resistance alleles was confirmed by PCR analysis using TMD1 an indel marker in the RLK intron. Several designs of TMD1 primers have been reported [2, 12, 47]. Used here were the primers pair: Rhg1/Rfs2-TMD1-F: 5′-CAC CTG CAT CAA GAT GAA CA-3′ and Rhg1/Rfs2-TMD1-R: 5′-GCC TAT TAC TTG GGA CCC AA-3′. PCR conditions were 35 cycles of 95° C. for 15 sec, 60° C. for 1 min.

### Example 7

### Allele Distribution in Soybean Germplasm

Genotypes at TMD 1 were determined from the genomic DNA of 112 Plant introductions that represented the sources of SCN and SDS resistance in World germplasm. Three of these represented the R parents of populations in the SIUC soybean breeding program from 1997-1999 (Peking PI88788 and PI437654). There were 80 cultivars somewhat susceptible to SCN race 3 and 22 PIs resistant to SCN race 3. Allele 2 (R) was found in 22 of 22 resistant PIs tested. There were very few somewhat susceptible genotypes with allele 2 (8 of 80) and the majority of genotypes with allele 2 (22 of 30) were resistant to SCN. In contrast, allele 1 (S) was found in 80 PIs. DNA sequencing from 112 SCN-resistant PIs and 34 derived cultivars inferred nine rhg1 haplotypes, four of which were SCN resistant (Hauge et al. 2001; Afzal et al. 2004). Relatively few nucleotide substitutions were predicted to result in amino acid changes so that only five protein allotypes were predicted, 3 in the LRR domain. However, three potential QTNs in rhg1 were inferred. One alters A87 to V87, the second alters Q115 to K115 and the third alters H to N at position 274. The substitutions may alter pre-protein transport or protein function or both. A87 was only associated with type I (Peking) resistance. The presumed rhg1 gene haplotypes were separated by 17 SNPs (Single Nucleotide Polymorphisms; Hauge et al. 2001) and two insertions/deletions in minisatellite markers SattTMD1 and SIUC-ScaS (Ruben et al. 2006).

### Example 8

### Selection of FRR/CN/SDS Resistant Seeds

G. max L. seeds used to start cultures should be less than six months old and have been stored in darkness at 4° C. Then, the seeds are cultured as folllows:

1. Surface disinfect with 70% (v/v) ethanol for 2 min then 20% (v/v) bleach for 20 min. Rinse three times in sterile MS media.

2. Germinate the seed on MS media containing 10 g/l agar, 30 g/l sucrose but no PGRs for 3 days at 27° C.

3. Axenically remove the testa, remove the cotyledonary notes, cut the cotyledons transversely in half and use the distal cotyledonary halves to establish callus cultures.

To initiate callus growth, cotyledonary halves are placed on MS medium with 30 g/l sucrose, 5 mM kinetin, 100 mg/l myoinositol, 0.5 mg/mL thiamine.HCl pH 5.7 at 27° C. unless noted below. The medium contains 5 mM indolebutyric acid as auxin. Place cotyledonary halves in tubes containing 10 mL solidified media. Incubate for 28 days.

To assay callus growth, pieces of callus each approximately 25 mg should be added to sterile tubes containing 10 mL media with varying concentrations of H. glycines, F. virguliforme or extracts thereof. After 28 days at 28° C. the explants are evaluated for growth and growing sectors subcultured.

Cell suspensions are derived by placing 2 g of a macerated callus in 40 mL of MS medium. The flask, a 125 mL Erlenmeyer flask, should be capped with a foam plug. Subcultures should be made every 14 days into fresh media by allowing the cells to settle, removing the old media by aspiration, adding twice the volume of fresh media and splitting into two flasks.

Soybean tissue capable of regeneration to whole plants are grown in the presence of H. glycines, F. virguliforme or extracts thereof. Cell lines representing mutants capable of continued growth are regenerated and the heritability of FRR, CN or SDS resistance determined in these plants or their seed or tissue derived progeny.

### Example 9

### Soybean Transformation with the RLK Gene in PSBHB94 and Resistance to FRR/CN/SDS

For soybean transformation, the cassette included SBHB94 that was a 9.772 kbp insert sub-cloned from BAC B21d9 by nebulization, size fractionated to 9-11 kbp and ligated into pHOS2. Transformation, selection and plant regeneration were conducted as in (Simmonds, 2003). Briefly, proliferative embryogenic cultures of soybean cv. X5 (AAFC breeding line X2650-7-2-3) or ‘Westag 97’ were co-bombarded with the pHOS_SBHB96 and Hyg^{R} (Gritz and Davies 1983) constructs; transgenic events were selected and maintained on 55 mg L⁻¹ hygromycin; embryos were matured on antibiotic-free medium, air desiccated and converted on B5 medium (Gamborg et al. 1968); tissue cultures and regenerating plantlets were maintained at 20 C and 20 h photoperiod. The plantlets were transferred to soil and plants were regenerated under controlled conditions as in (Simmonds 2003). Primary transgenic (Tₒ) plants were tested for the presence of the pHOSSBHB96 transgene using PCR with the TMD1 primers. T₂ seed from T₁ plants was tested for transgene segregation to identify homozygous T₁ individuals.

The genotype of X5 and Westag 97 were both rhg1/rfs2rhg1/rfs2, rhg4rhg4. Purified stable transgenics were of genotype rhg1/rfs2rhg1/rfs2, rhg4rhg4:: Rhg1/Rfs2Rhg1/Rfs2 as shown by markers TMD1 and A2D8. Expression of the transgene was established by RT-PCR from cDNA with allele specific Taqman probes and HRM of amplicons. Protein allotypes were identified by two dimensional PAGE followed by Western hybridization (Afzal et al. 2007).

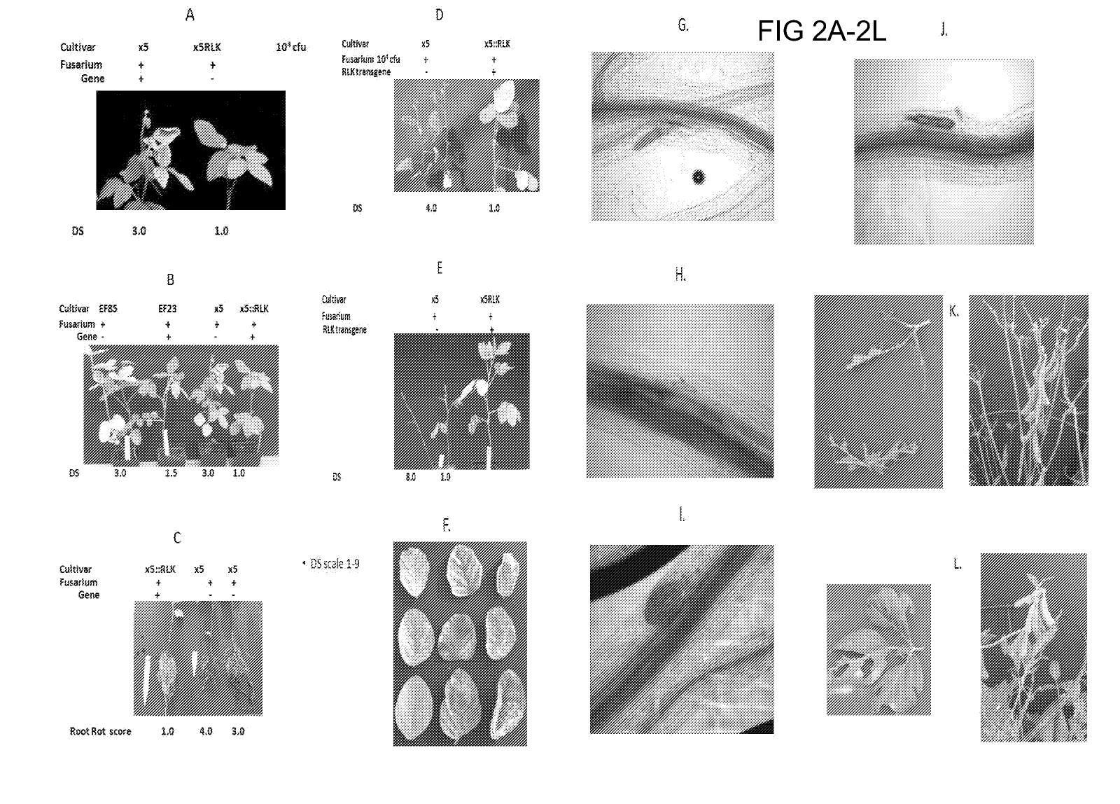

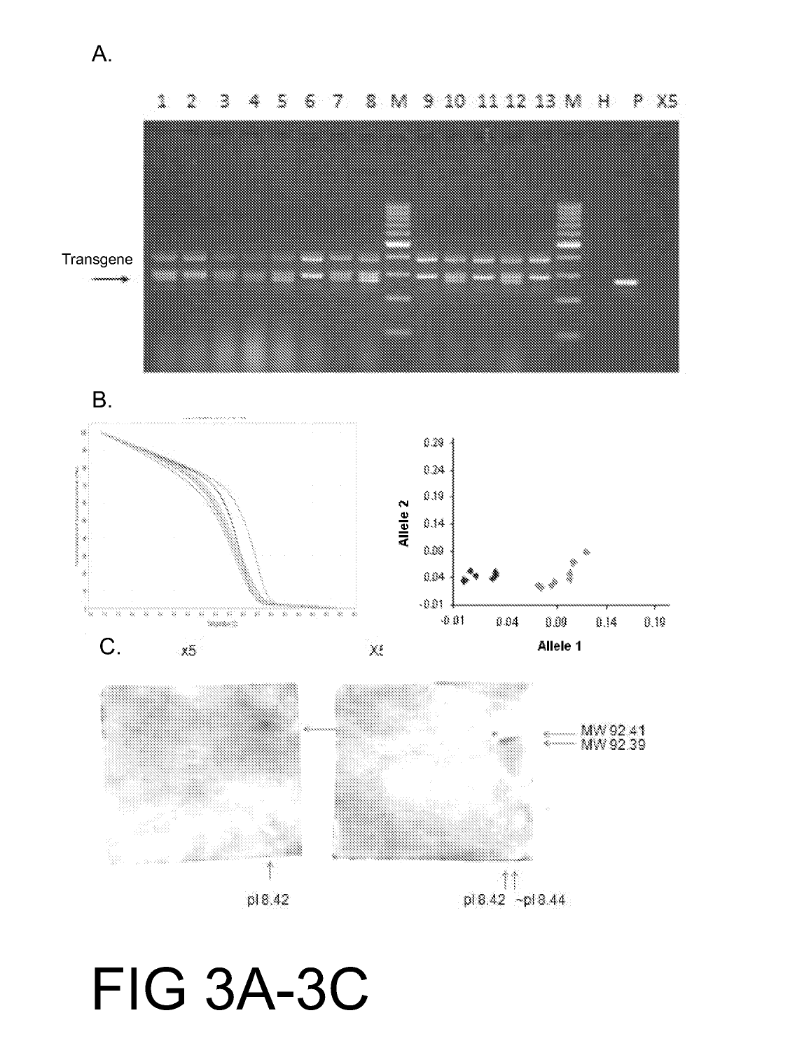

In several lines and backcross/selfed lines the RLK transgene was present in the homozygous state (FIG. 3). The transgene was expressed as both mRNA and protein from the native promoter contained on the 9.772 kbp fragment from BAC B21d09 (FIG. 1; HQ008939; SEQ ID NO; 3). Assays of SDS showed that the RLK provided resistance to root infection and root rot by Fusarium virguliforme. That root resistance underlay a significant reduction in leaf symptoms and delay of the senescence caused by SDS (FIG. 2; Table 3). Resistance to SDS was effective throughout the life of the plant which flowered and set seed. The non-transgenic X5 plants proved to be highly susceptible to SDS and showed all the expected phenotypes of root rot and leaf scorch. The non-transgenic Westag 97 plants were moderately resistant to SDS but were very susceptible to root rot by F. virguliforme. The phenotypes among the susceptible plants included a gradual worsening of leaf scorch symptoms from 3.0 at 21 dai to 8.5 by 56 dai. Near maturity the susceptible plants showed all the worst symptoms characteristic of SDS like leaflet abscission from the top of the petiole instead of the base of the petiole, early senescence and reduced pod set. Root symptoms characteristic of SDS included root rot and browning of the root cortex. The RLK provided a very high degree of resistance to the transgenic plants in both roots and leaves. Senescence was delayed by 14 days compared to controls in each of the three repeats of the experiment. Leaflet abscission was normal and root mass was normal.

The resistance to SCN was partial (Table 3) as judged by female number. SCN FI was reduced by 30-50% across four experiments using three Hg Types (P<0.01). Since the transgenic plants reported here had a susceptible allele at Rhg4 partial resistance was the expected outcome. Nematode development was arrested by about 10 dai (FIG. 3) compared to the controls. The partial resistance was confirmed with a third isolate of SCN at Harbin University (China). Therefore, the RLK was alone sufficient to provide for an Rhg1-like activity. The linked genes appear to reduce SCN numbers but do require the action of a second locus (Rhg4) or linked gene from the region encompassed by BAC73P6 (SEQ ID NO; 4) to provide full resistance to Hg Type 0 (FIG. 2; Table 3).

Table 3: Association of mean root growth in transgenic lines with pleiotropic resistance to two pests in two different greenhouse assays and insect herbivory in field tests. Part A shows SCN female index in greenhouse grown seedlings at 28 days after SCN infestations. Pots were watered daily with 100 ml. Female index (FI) was the mean percentage of cysts of Hg Type 0 found on five plants per repetition compared to a susceptible genotype Essex. Part B shows the effects of the transgene on resistance to F. virguliforme in greenhouse grown seedlings at 28 days after infestations. Pots were saturated with water to the 5 cm level. Leaf scorch was recorded as the mean disease severity (DS) measured on a 1-9 scale found on five plants per experimental repeat. Root rot severity (1S) was measured on a 1-5 scale. The experiments were repeated 4 times over 2 years. Panel C shows the percent insect incidence, defoliation by herbivorous insects and the consequent loss of biomass at harvest as mean dry weight per plant for field grown plants with 4 replications across 2 years.

| | | | Signif- | | | |
|---|---|---|---|---|---|---|
| | SCN | Root | icant | | | |
| A. | in- | mass | differ- | Range | | SCN |
| Line::gene | fested | (g) | ences | (g) | n | FI (%) |
| X5 | No | 1.05 | a | 0.81-1.44 | 15 | 0 ± 0.0 |
| X5 | Yes | 0.98 | a | 0.73-1.31 | 15 | 100 ± 13 |
| X5 plus CLE | Yes | 1.4 | a | 0.9-1.8 | 5 | 10 ± 6 |
| X5::RLK | No | 0.64 | bc | 0.57-0.74 | 15 | 0 ± 0.0 |
| X5::RLK | Yes | 0.38 | c | 0.26-0.49 | 15 | 60 ± 11 |
| X5::RLK::Rhg4 | Yes | nd | | | 2 | 11 ± 3 |
| X5::RLK::rhg4 | Yes | nd | | | 3 | 38 ± 6 |
| Westag97 | Yes | 4.2 | | 3.5-4.8 | 5 | 120 ± 13 |
| Westag97::RLK | Yes | 3.1 | | 2.6-3.5 | 4 | 5 ± 3 |

### Example 10

### Field Trials of Transgenic Plants

Field trials were conducted at the ARC in Carbondale in 2010 and 2011 using conditions described in (Triwitayakorn et al. 2005, Genome/Génome 48: 125-138). Plants were arrayed in a RCB. Insect herbivory was measured as described in (Yesudas et al. 2010, Theor Appl Genet 121:353-362). Briefly, the pest incidence was calculated as the number of individual plants within a given line that were affected by herbivorous insects; the pest severity was the percent defoliation; and both were measured once a week from the R1 to R6 growth stages.

Field grown plants showed that the RLK was associated with increased susceptibility to insect herbivory (Table 3; FIG. 2). Insects were attracted to the RLK transgenic plants judged by pest incidence (PI) measured from R1-R7 growth stages in both 2010 and 2011. Herbivory in 2010 resulted in plants with less leaf area that produced less shoot biomass and less seed at harvest. However, in 2011 herbivore pressure was lesser and the RLK transgenes was associated with more shoot biomass and more seed at harvest. Therefore, the gene can increase the yield of soybean and other crops. The linkage drag of rhg1 on yield reported for the past 40 years (reviewed by Concibido et al Crop Science 2004) has been broken in the transgenic plants.

### Example 11

### Total Root Protein Extraction, SDS-Page and Western Hybridization with a Specific Anti RLK Antibody

Protein from root material was isolated from infested and non-infested roots was extracted after (Afzal et al. 2009, Plant Physiology). Total protein concentration was determined using a non-interfering protein assay. For the Western hybridizations, a custom made antibody generated against a peptide CTL SRL KTL DIS NNA LNG NLP ATL SNL S from the LRR domain of RLK at RHG1/RFS2 was used (Alpha diagnostics, San Antonio, Tex.).

The presence of the rhg1 mRNA and protein in plant tissue was confirmed by RT-PCR and Western hybridization. The rhg1 transcript was detected under both inoculated and uninoculated conditions in the resistant cultivar Forrest and the susceptible cultivar Essex (FIG. 3). qPCR results to determine differences in transcript abundance between infected and uninfected cultivars were inconclusive. However, MS detection of the protein predicted from the gene sequences have been made both in vitro and in vivo.

Western hybridizations that used polyclonal antibodies generated against the RHG1-LRR yielded a single positive protein band (1D) or spot (2D) in Essex and Forrest (FIG. 3). Cloned and expressed RHG1-RLK18-1_LRR domain was used as the positive control whereas cloned and expressed RHG4-RLK8-1_LRR domain was used as a negative control.

### Example 12

### Purification of the RLK LRR Domain for Ligand Binding Assays

The full length rhg1/Rfs2 or Rhg4 cDNA from the SCN resistant cultivar, ‘Forrest’ (Bruker et al. 2005), was sub-cloned in pGEM-T vector. For the rhg1/Rfs2 LRR DNA was PCR amplified using internal primers. Primers were designed to copy and amplify a 1032 bp region encompassing the 10 LRRs of rhg1. The sense primer 141-485F: 5′-CCAATTCCATGGCGATCGAAGGTCGTCTTCAAGGCCTCAGG-3′ contained an eight base linker followed by an NcoI restriction site (in bold) to allow in-frame cloning into the expression vector pET30A (+). A factor Xa site (underlined) was incorporated in the sense primer in order to cleave the fusion protein. The anti-sense primer 141-485R: 5′-CGGTTTCTCGAGCTATTAGAGAATTATGTCTTTGGTGCTTAG-3′ contained a six base linker followed by an XhoI restriction site (in bold). The stop codon (underlined) was added to the primer sequence to terminate the RHG1 translation. The amplicon obtained by PCR was isolated by agarose gel electrophoresis and purified using gel elution. The amplified fragment was subcloned into pGEMT vector by ligation of the 3′T vector overhang with the 3′A added by Taq polymerase during template amplification. The complementarity between the vector 3′T overhangs and the PCR product 3′A overhangs allowed for direct ligation of PCR products into the vector. pGEMT vector was transformed into DH5a cells, positive colonies were selected and grown overnight in 5 ml of 2-fold YT medium. The recombinant plasmid DNA was purified using the Qiagen mini-prep kit.

Cloning of RHG1-LRR-Long-Histidine (RLLH) in the pET30A Expression Vector

About 10 g of the LRR sub-clone plasmid DNA was digested with NcoI and XhoI. Purified insert was ligated overnight at 16° C. using T4 DNA ligase into a XhoI/NcoI predigested pET30a (+) vector. The ratio of insert DNA to plasmid DNA was 3:1. The construct contained the hexa-his inframe with the Factor Xa vector site and 5′ end of the insert. The recombinant plasmid was transformed into DH5a cells. Positive colonies were selected on 2-fold YT plates supplemented with 50 μg/ml kanamycin and screened by digestion of mini-prep plasmid DNA with NcoI and XhoI followed by agarose gel electrophoresis. The nature of the inserts were also verified by DNA sequence analysis using the ABI 377 DNA sequencer (Foster City, Calif.).

### Generation of RHG1-LRR-Short-His Rhg1 (RLSH)

A Quikchange site directed mutagenesis kit (Stratagene) was used for RLSH generation. Both sense and anti-sense primers were designed to loop out the 114 bp nucleotide linker region between the hexa-his tag and the first codon of RLLH. The forward primer DMF1: 5′-CACCATCATCATCATCATCTTCAAGGCCTCAGGAAG-3′ and reverse primer DMR1: 5′-CTTCCTGAGGCCTTGAAGATGATGATGATGATGGTG-3′ were complementary in sequence and annealed to opposite stands of DNA. Amplification of template was performed with Pfu-Turbo DNA polymerase. Following amplification, the parent DNA (methylated and hemi-methylated) was digested with DpnI endonuclease (target sequence: 5′-Gm6ATC-3′). The amplified vector DNA containing the desired deletion was transformed into XL1-Blue competent cells and the cells were grown overnight in 2XYT supplemented with 50 μg/ml tetracycline and 50 μg/ml kanamycin. Colonies were picked, grown overnight, DNA purified and inserts identified by digestion of plasmid DNA with NdeI and XhoI followed by 2% (w/v) agarose gel electrophoresis. DNA was subsequently sequenced to verify looping out of the RLLH linker region. DNA from positive clones was transformed into BL21 (DE3-RIL) competent cells.

RHG1 was expressed in BL21 (DE3-RIL) cells

BL21-CodonPlus™-RIL series of strains contain extra copies of the E. coli argU, ileY, and leuW tRNA genes. The modification allowed for high expression of proteins that were difficult to express in conventional E. coli hosts due to the codon usage bias of the gene of interest. RHG1 protein contained 18 codons rarely used in E. coli in the LRR alone (Table 3.1). The 5 ml of LB overnight culture containing Rhg1/Rfs2 LRR or Rhg4 LRR was transferred to 1 L of M9 minimal media supplemented with ammonium sulfate and kanamycin. The cells were grown at 37° C. to an O.D of 1.0. The protein was induced with 0.4 mM IPTG and incubated at 16° C. in an orbital shaker for 18-20 h.

### Identification of Interacting Proteins and Peptides

Proteins were solubilized from IBs, purified and refolded by the method described by Afzal and Lightfoot 2007, Protein Expression and Purification 53: 346-355. They were produced as in Example 9 and used for Far Western identification of interacting root proteins (FIGS. 4 & 5) and for identification of preferred ligands among small peptides found in root phloem (Table 4). Far-Western analyses followed by MS identification of tryptic peptides suggest both cyclophilin SEQ ID NO; 22 and methionine synthase SEQ ID NO; 23 bound strongly to the LRR domain. A second LRR from GmRLK08-1 did not show these strong interactions as described by Srour et al 2012, BMC Plant Biology.

Assays in vitro showed binding constants of 20-142 nM for peptides found in plant and nematode secretions SEQ ID NO: 24-32. There were 5 separate motifs among the eight GmCLE peptides as 3 of the motifs were present in short (12 residues) or long peptides (28-32 residues). The GmRLK18-1-LRR had highest affinity for short peptides in general (14-45 nM) and longer peptides were 2-3 fold less strongly bound. Peptides GmCLE34 (14 nM) and T (20 nM) were bound most strongly followed by CLV3 and its nematode ortholog N (29-30 nM). These binding constants were within physiological ranges and suggest the LRR domain can bind multiple ligands. Each of the ligands was found in vivo as part of a signal cascade that alters plant development [25-29]. In contrast, to the set of peptides associated with developmental controls the peptides involved in the control of nodule symbiosis GmRIC1 and GmNIC1 were bound weakly by the GmRLK18-1 derived LRR peptide.

The GmRLK8-1 LRR domain (from the RLK protein at Rhg4) showed a lower affinity for most of the CLE peptides tested (50-338 nM). However, the long and short versions of GmCLE34 and short version of GmCLV3 bound with the highest affinity (50-52 nM) suggesting these were the natural ligands. The nematode peptide HgCLV3 was bound weakly (78 nM). This result would agree with the conclusion that GmRLK8-1 protein was not the sole element underlying the resistance reaction encoded at the Rhg4 locus [9]. The GmRLK8-1 LRR domain protein bound very weakly to the symbiosis associated GmRIC and GmNIC, as did GmRLK18-1. Unlike GmRLK18-1 the GmRLK8-1 protein bound weakly to GmTDIF. Therefore, the peptides showed distinct ligand specificities reflecting their different sequence and structures.

Estimates of the Kd for dimerization could be made from the peptide L which contained one diverged LRR motif. The apparent Kd for dimerization of 36 nM for this region would suggest the whole domain homo-dimerization constant be less than that. In vitro both proteins extracted from roots and LRR domain peptides solubilized from E. coli showed evidence that about half the proteins existed as monomers and about half as homo-dimers. This equilibrium is maintained across a wide range of concentrations of protein and salt concentrations. It will be of interest in future to see if ligand binding can alter this equilibrium.

CLE-like protein derived consensus peptides are defined set of peptides found in plant genomes and involved in both short and long distance signaling. The GmRLK18-1 LRR domain had a strong binding constant for GmCLV3 and N that are thought to be involved in meristem differentiation. During SCN pathogenesis a new meristem is initiated to bring a tracheary element close to the feeding site, N might mediate that and be detected by the resistance protein GmRLK18-1. T is the tracheary element differentiation inhibitory factor (GmTDIF) that might provide inhibition of feeding site induced developmental processes during defense. GmCLE34 peptides were produced in pro-vascular tissues [47]. CLE domains thought to be involved in symbiosis were not strongly bound suggesting they were not ligands of physiological relevance although nematode parasitism does decrease nodulation.

Plant treatments with TGIF and N were found to be effective as exogenous treatments. Phenotypes observed following treatments included transient wilting and increased plant vigor associated with increased disease resistance. It was concluded the peptides might be effectively used to control SDS and SCN as foliar sprays of seed and root treatments.

TABLE 4
Sequences of CLE like peptides and control peptides used in
binding assays. Consensus sequences within the peptides were
underlined. Dashed underline was the LRR peptide fragment used to
estimate the Kd of dimerization and to raise the anti-Gm18RLK-1
antibody. Complete annotations can be found in Oelkers et al. (2008).
Binding constants were calculated from titration experiments.
Luminiscencse Spectrometry used parameters:Start (nm): 500;
End (nm): 600; Excitation Energy (nm): 496; Excitation Emmision
Energy: 517.73; Excitation slit: 4 (nm); Emission Slit: 4 (nm);
Scan speed nm/min: 100.

| | Kd (nM) | | | |
|---|---|---|---|---|
| Sequence | Name | Synonyms | RHG1 | RHG4 |
| RLAPGGPDPQHN | 2 | GmNIC1, LjCLE-R2 and LjCLE-R1 | 45 | 96 |
| DLPLAPADRLAPGGPDPQHNVRAPPRKP | 2L | GmNIC1, LjCLE-R2 and LjCLE-R1 | 142 | 338 |
| RLAPEGPDPHHN | 30 | GmCLE30, GmRIC1 | 44 | 84 |
| AHEVPSGPNPISNR | T | GmTDIF, ZeTDIF | 20 | 204 |
| SKRRVPNGPDPIHNR | 36 | GmCLE34, AtCLE36, MtCLE36 | 14 | 52 |
| RAELDFNYMSKRRVPNGPDPIHNRRAGNSGR | 36L | GmCLE34, AtCLE36, MtCLE36 | 49 | 51 |
| RTVPSGPDPLHH | 3 | GmCLE3, AtCLE3, AtClv3 unmodified | 29 | 50 |
| KGLGLHEELRTVPSGPDPLHHHVNPPRQPR | 3L | GmCLE3, AtCLE3, AtClv3 unmodified | 65 | 142 |
| KRLSPSGPDPHHH | N | HgCLV3 | 30 | 78 |
| CTLSRLKTLDISNNALNGNLPATLSNLS | L | GmLRR, GmRLK18-1 | 36 | 135 |

### Example 13

### Proteins Altered in Abundance by the Allele at Rhg1/Rfs2

Proteins altered in abundance by the Rhg1/Rfs2 locus and the Gm18-1 gene were identified by 2d gel electrophoresis after Afzal et al 2009 Plant Physiology in response to SCN and compared to those changed in response to F. virguliforme (unpublished) as follows.

2D gel electrophoresis and image analysis

Total protein extract (275 μg) from inoculated NILs (34-3 or 34-23) and un-inoculated NILs 34-23 and 34-3 was used for the 21) electrophoretic analysis. Sample was initially hydrated overnight on a 17 cm BioRad IPG gel strips with a 3-10 pH gradient. The next day IEF (Iso-electric focusing) was performed with the Protean IEF Cell (BioRad, Hercules, Calif.). Equilibration of the strips was according to the manufacture's instructions. Linear SDS PAGE gels (8% to 16% w/v) were used for resolution of proteins in the second dimension. A BioRad Protean II apparatus was used for gel electrophoresis at 15 mA/gel for 30 min, followed by 25 mA/gel for approximately 5 hrs at ambient temperature (20±2° C.). Gels were washed with distilled water and stained with SYPRO Ruby.

### Image Analysis and Spot Picking

Acquisition of gel images used a high resolution laser scanner (Typhoon 9410 from GE Healthcare, Piscataway, N.J.) and a CCD camera linked to a GelPix protein spot excision system (Genetix, New Milton, UK). Images were analyzed with the Imagemaster 2D software (GE Healthcare). The analysis was fully automated unless there was a need. to edit unresolved spots. Identical spots from the compared gels were chosen by assigning landmarks to each gel. The volume for each protein spot was normalized against the total spot intensity. Students T-test (p, 0.05) was used to determine whether spot intensities between treatments were significantly different, Differentially abundant spots were excised manually or with the GelPix system.

### Protein Digestion and Ziptip Clean-Up

Proteins were digested in-gel as described previously (Gabelica et al. 2002), with the exception that digestion was carried out at 37° C. overnight with 6 mg/ml trypsin in 50 mM NH₄HCO₃. The samples were initially extracted with 30 μl of 1% (v/v) formic acid, 2% (v/v) methyl-cyanide followed by incubation at 30° C. for 30 min on a shaking platform. For the second extraction, 60% (v/v) acetonitrile was used. The pooled extractions were lyophilized and stored. 2% (v/v) acetonitrile, 1% (v/v) formic acid solution was used for sample resupension. Samples were cleaned using a 10 μl C18 ZipTip according to manufacturer.

### Database Searching

Proteins were identified via peptide sequencing using ESI MS/MS as described previously (Chen et al. 2005). Analyst QS software (Applied Biosystems) was used for spectral processing. The peptides were searched against the Soybean and Medicago truncatulata EST databases (downloaded from NCBI, January 2006), the non-redundant NCBI database, and the Swissprot database using MASCOT version 1.9. Input parameters for variable and fixed modifications were specified as “oxidation of methionine and carbamidomethylation of cysteine” respectively. Positive identification was based on; (a) number of peptide sequences identified in a protein; (b) calculated and theoretical PI/molecular weight; and (c) total MASCOT and MOWSE scores (http://www.matrixscience.com/help/scoring_help.html) at a p value of 0.05. Protein sequences derived from EST databases or without significant matches were searched against the NCBI blastx, tblastx database to detect possible orthology.

Protein identifications could be made for 24 out of the 30 spots. Four spots contained two proteins so that 28 distinct proteins were identified (Afzal et al., 2009,). The proteins were grouped into six functional categories. Metabolite analysis by GC-MS identified 131 metabolites among which 58 were altered by one or more treatment, 28 were involved in primary metabolisms. Taken together the data showed seventeen pathways that were altered by rhg1 controlled metabolisms associated with SAR-like responses including xenobiotic, phytoalexin, ascorbate and inositol metabolism as well as primary metabolisms like amino acid metabolism and glycolysis. The pathways impacted by the rhg1 allelic state and SCN infestation agreed with transcript abundance analyses but identified a smaller set of key proteins. Six of the proteins lay within the same region of the interactome identifying a key set of 159 proteins inferred to be involved in nuclear protein transport and degradation. Finally, two proteins (glucose 6 phosphate isomerase, EC 5.3.1.9; and isoflavone reductase, EC 1.3.1.45) and two metabolites (maltose and unidentified) differed in resistant and susceptible NILs without SCN infestation and may form the basis of a new assay for the selection of resistance to SCN in soybean.

The proteins altered in abundance when F virguliforme was infesting were different (Table 5; unpublished). Analysis of the proteome of transgenic plants showed that proteins involved in the salicyclic acid pathway of the systemic acquired resistance pathway were increased in abundance by Rhg1/Rfs2 whereas protein involved in the jasmonic acid pathways were reduced in abundance (Supplemental Table 1). Interpathway interference may have caused the insect susceptibility since the major insect guilds (chewers and suckers) were known to differ in the defense pathways induced. Inter-pathway interference may explain why the Rhg1/Rfs2 locus was complex and highly regulated.

TABLE 5
Proteins altered in abundance by more than 2 fold in soybean roots with the Resistant
allele of Rfs2 when infested by Fusarium virguliforme

| Accession No. | Identified protein annotations | MOWSE | Function/role |
|---|---|---|---|
| gi\|1276977 | Non-symbiotic hemoglobin | 143 | Oxygen carrier or sensor, electron transport |
| gi\|31468505 | EST increased by salicylic acid | 56 | Stress response |
| gi\|6847700 | Dirigent-like protein | 158 | Disease response |
| gi\|18733747 | Zn finger protein | 52 | Transcriptional regulation |
| gi\|1498330 | Actin | 200 | Intracellular trafficking and cold stress |
| gi\|21479786 | Pectin-esterase 2 | 252 | Cell wall metabolism in flooded roots |
| gi\|37996530 | Expansin B1 like protein | 242 | Stress related, P. sojae infestation |
| gi\|18744 | Stress-induced protein SAM22 | 312 | Stress related |
| gi\|1262132 | PR1 | 312 | Stress, SAR and pathogensis related protein |
| gi\|22296818 | Pyruvate kinase | 187 | Glycolysis, fatty acid biosynthesis |
| gi\|4204761 | Peroxidase precursor | 104 | Stress reduction/antioxidant |
| gi\|4292702 | Endopeptidase beta | 148 | Protein degradation and global gene regulation |
| gi\|6847700 | PR protein | 151 | Stress, SAR and pathogensis related protein |
| gi\|4287344 | Calmodulin | 111 | Calcium-binding protein |
| gi\|134194 | SAM22 | 110 | Stress tolerance |
| gi\|82408517 | PR-1 | 83 | Disease and pathogenesis-related protein |
| gi\|14126046 | Pectin-esterase 2 | 224 | Cell wall metabolism |
| gi\|12710364 | Oxysterol-binding protein | 25 | Sterol synthesis and metabolism |
| gi\|120532 | Ferritin-1 | 83 | anion homeostasis |
| gi\|38192463 | Triose phosphate isomerase | 264 | Glycolysis |
| gi\|18728 | chalcone synthase | 196 | Flavonoid biosynthesis |
| gi\|10946357 | Glutamine synthetase beta1 | 390 | Nitrogen metabolism |
| gi\|6846308 | Peroxidase | 190 | Stress reduction |
| gi\|2578440 | Similar to pectin-esterase | 116 | Cell wall metabolism |
| gi\|54035683 | Actin | 398 | Intracellular trafficking and cold stress |

The abundance of any of these proteins can be used as biomarkers of resistance to SDS and allow for marker assisted selection based on protein or DNA probes.

### Example 14

### Negative Growth Effects on Yield of the Transforming BACs B73P06 and H38F230N the Growth of a Brassica, Arabidopsis Thaliana

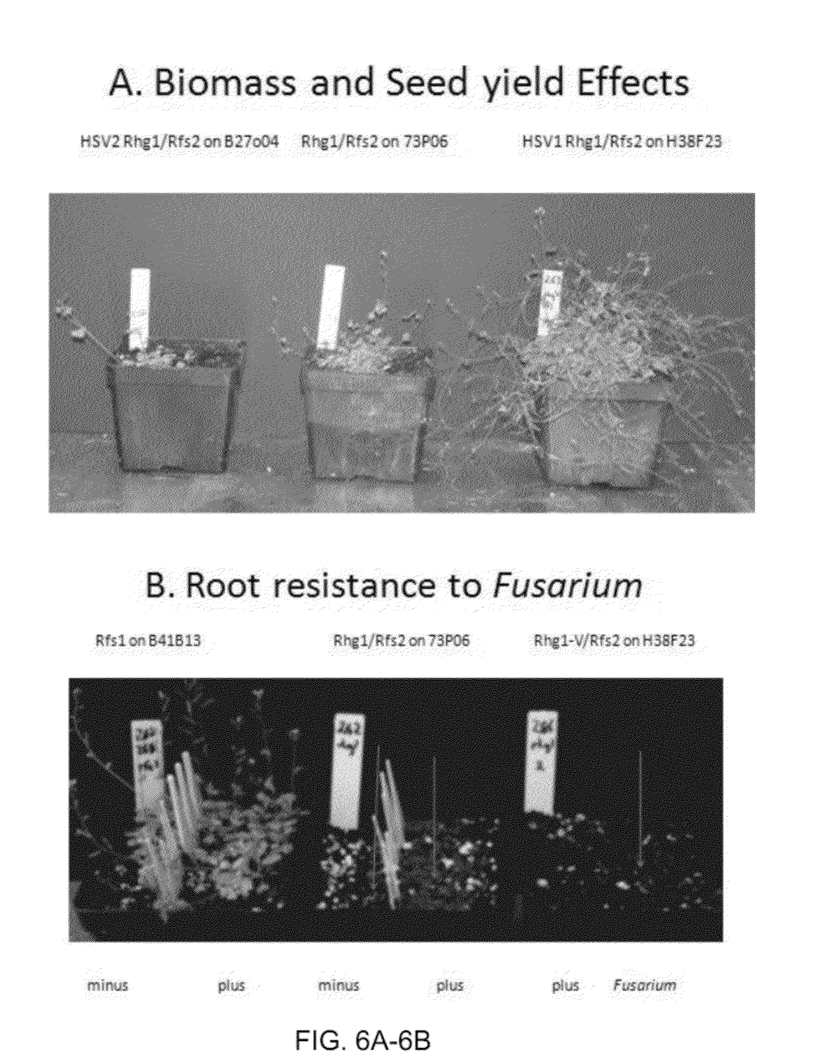

Transformed A. thaliana plants were produced as described by Ullah, Jasim and Lightfoot 2012 ATLAS J Plant Genome. BACs B73P06 (SEQ ID NO; 4) reduced biomas and seed yield significantly (FIG. 6). Therefore, the element(s) causing the linkage drag on yield are present on the BAC but were removed from pSBHB94 (SEQ ID NO; 3). In contrast the syntenic homologous BAC H38F23 (SEQ ID NO; 34) increased biomass and seed yield showing the yield drag elements were distal to the RLK gene.

### REFERENCES

The publications and other materials listed below and/or set forth in the text above to illuminate the background of the invention, and in particular cases, to provide additional details respecting the practice, are incorporated in their entirety herein by reference. Materials used herein include but are not limited to the following listed references.
1. Ablett, G. R., Stirling, B. T. and Fischer, J. D. 1999. Westag 97 Soybean. Can. J. Plant Sci. 79: 371-372.
2. Adelman et al. (1983) DNA 2:183-193.
3. Alam and Cook (1990) Anal Biochem 188:245-254.
4. Altschul et al. (1990) J Mol Biol 215:403-410.
5. Arondel et al. (1992) Science 258:1353-1355.
6. Ausubel et al. (1992) Current Protocols in Molecular Biology, John Wylie and Sons, Inc., New York.
7. Bartlett et al. (1982) in Methods in Chloroplast Molecular Biology, Edelmann et al. (Eds.), pp 1081-1091, Elsevier.
8. Barton (1998) Acta Crystallogr D Biol Crystallogr 54:1139-1146.
9. Batzer et al. (1991) Nucleic Acid Res 19:3619-3623.
10. Bell-Johnson et al. (1998) Soybean Genet Newslett 25:115-118.
11. Bernard et al. (1988) USDA Tech Bull 1796.
12. Bevan (1984) Nucl Acids Res 12:8711-8721.
13. Bevan et al. (1983) Nature 304:184-187.
14. Binet et al. (1991) Plant Science 79: 87-94.
15. Blochlinger & Diggelmann (1984) Mol Cell Biol 4:2929-2931.
16. Bourouis et al., (1983) EMBO J2(7):1099-1104.
17. Bodanszky, et al. (1976) Peptide Synthesis, John Wiley and Sons, Second Edition, New York.
18. Brookes (1999) Gene 234(2):177-186.
19. Brown et al. (1987) Principles and Practice of Nematode Control in Crops, pp 179-232, Academic Press, Orlando Fla.
20. Callis et al. (1987) Genes Develop 1:1183-1200.
21. Chang et al. (1996) Crop Sci 36:965-971.
22. Chang et al. (1997) Crop Science 37(3):965-971.
23. Chase et al. (1997) Theor Appl Genet. 94:724-730.
24. Chibbar et al. (1993) Plant Cell Rep 12:506-509.
25. Christou et al. (1991) Biotechnology 9:957-962.
26. Comai et al. (1988) J Biol Chem 263:15104-15109.
27. Concibido (1996) Theor Appl Genet. 93:234-241.
28. Concibido et al. (1997) Crop Sci 37:258-264.
29. Conner et al. (1983) Proc Natl Acad Sci USA 80:278-282.
30. Cregan et al. (1999a) Crop Sci 39:1464-1490.
31. Cregan et al. (1999b) Theor Appl Genet. 99:811-818.
32. Cregan et al. (1999c) Theor Appl Genet. 99:918-928.
33. Cubitt et al. (1995) Trends Biochem Sci 20:448-455.
34. Datta et al. (1990) Biotechnology 8:736-740.
35. EP 0 292 435
36. EP 0 332 104
37. EP 0 332 581
38. EP 0 342 296
39. EP 0 392 225
40. EP 0 452 269
41. Fehr (1987) in Soybeans: Improvement Production and Uses 2nd Ed., J. R. Wilcox (ed.), American Society of Agronomy, Madison, Wis.
42. Firek et al. (1993) Plant Molec Biol 22:129-142.
43. Fromm et al. (1990) Biotechnology 8:833-839.
44. Gallie et al. (1987) Nucl Acids Res 15:8693-8711.
45. Glover, ed. (1985) DNA Cloning: A Practical Approach, MRL Press, Ltd., Oxford, United Kingdom.
46. Gomez A. K. and A. A. Gomez (1984) Statistical Procedures For Agricultural Research. 2nd ed. John Wiley & Sons New York
47. Gordon-Kamm et al. (1990) Plant Cell 2:603-618.
48. Gritz et al. (1983) Gene 25:179-188.
49. Harlow and Lane (1988) Antibodies: A Laboratory Manual, Cold Spring Harbor Laboratory Press, Cold Spring Harbor, N.Y.
50. Hartwig and Epps (1973) Crop Science 13:287.
51. Henikoff et al. (2000) Electrophoresis 21(9):1700-1706.
52. Henikoff and Henikoff (1989) Proc Natl Acad Sci USA 89:10915.
53. Henikoff and Henikoff (2000) Adv Protein Chem 54:73-97.
54. Hnetkovsky et al. (1996) Crop Science 36(2):393-400.
55. Hofgen & Willmitzer (1988) Nucl Acids Res 16:9877.
56. Huang et al. (2000) Pac Symp Biocomput 230-241.
57. Hudspeth & Grula (1989) Plant Molec Biol 12:579-589.
58. Hutchens and Yip (1993) Rapid Commun Mass Spectrom 7: 576-580.
59. Kalinina et al. (1997) Nucl Acids Res 25:1999-2004.
60. Kanazin et al. (1996) Proc Natl Acad Sci USA 93(21):11746-11750.
61. Karlin and Altschul (1993) Proc Natl Acad Sci USA 90:5873-87.
62. Keim et al. (1997) Crop Science 37:537-543.
63. Kestila et al. (1998) Mol Cell 1(4):575-582.
64. Klein et al. (1987) Nature 327:70-73.
65. Koduri and Poola (2001) Steroids 66(1):17-23.
66. Koziel et al. (1993) Biotechnology 11:194-200.
67. Kyte et al. (1982) J Mol Biol 157:105.
68. Landers & Botstein (1989) Genetics 121:185-199.
69. Landgren et al. (1988) Science 241:1007.
70. Landgren et al. (1988) Science 242:229-237.
71. Landegren et al. (1998) Genome Res 8:769-776.
72. Lark et al. (1993) Theor Appl Genet. 86:901-906.
73. Li and Herskowitz (1993) Science 262:1870-1874.
74. Liedberg et al. (1983) Sensors Actuators 4:299-304.
75. Livak et al. (1995) PCR Meth and Applic 4:357-362.
76. Livak et al. (1995) Nat Genet. 9:341-342.
77. Logemann et al. (1989) Plant Cell 1:151-158.
78. Luo et al. (1999) Plant Disease 83:1155-1159.
79. Madge et al. (1972) Phys Rev Lett 29:705-708.
80. Mahalingam et al. (1995) Breed Sci 45:435-445.
81. Mahalingham et al. (1996) Genome 39:986-998
82. Maiti et al. (1997) Proc Natl Acad Sci USA, 94:11753-11757.
83. Malmquist (1993) Nature 361:186-187.
84. Martin et al. (1993) Science 262:1432-1436.
85. Mathews et al. (1998) Theor Appl Genet. 97:1047-1052.
86. Matthews et al. (1991) Soybean Genetics Newsletter.
87. McBride et al. (1990) Plant Molecular Biology 14:266-276.
88. McElroy et al. (1990) Plant Cell 2:163-171.
89. McElroy et al. (1991) Mol Gen Genet. 231:150-160.
90. Meksem et al. (1995) Mol Gen Genet. 249:74-81.
91. Meksem et al. (1999) Theor Appl Genet. 99:1131-1142.
92. Meksem et al. (2000) Theor Appl Genet. 101:747-755.
93. Messing & Vierra (1982) Gene 19:259-268.
94. Myers & Anand (1991), Euphytica 55:197-201.
95. Nasarabadi et al. (1999) BioTechniques 27:1116-1117.
96. Needleman & Wunsch (1970) J Mol Biol 48:443-453.
97. Njiti et al. (1996) Crop Science 36:1165-1170.
98. Ochman et al. (1990) in PCR protocols: a Guide to Methods and Applications, Innis et al. (eds.), pp. 219-227, Academic Press, San Diego, Calif.
99. Ohtsuka et al. (1985) J Biol Chem 260:2605-2608.
100. Orita et al. (1989) Proc Natl Acad Sci USA 86(8):2766-2770.
101. Paszkowski et al. (1984) EMBO J. 3:2717-2722.
102. Paterson et al. (1990) Genetics 124:735-742.
103. Pearson & Lipman (1988) Proc Natl Acad Sci USA 85:24442448.
104. Potrykus et al. (1985) Mol Gen Genet. 199:169-177.
105. Prabhu et al. (1999) Crop Science 39(4):982-987.
106. Price (1993) Blood Rev 7:127-134.
107. Rao-Arrelli et al. (1988) Crop Science 28:650-652.
108. Rao-Arrelli et al. (1992) Crop Science 32:862-864.
109. Regan et al. (2000) Anal Biochem 286(2):265-276.
110. Reich et al. (1986) Biotechnology 4:1001-1004.
111. Riggs and Schmidt (1988) J Nematol 20:392-395.
112. Rogers et al. (1989) Proc Natl Acad Sci USA 82:6512-6516.
113. Rohrmeier & Lehle (1993) Plant Molec Biol 22:783-792.
114. Rommens et al. (1989) Science 245:1059-1065.
115. Rose & Botstein (1983) Meth Enzymol 101:167-180.
116. Rossolini et al. (1994) Mol Cell Probes 8:91-98.
117. Rothstein et al. (1987) Gene 53:153-161.
118. Saiki et al. (1985) Bio/Technology 3:1008-1012.
119. Sambrook et al. eds. (1989) Molecular Cloning: A Laboratory Manual, Cold Spring Harbor Laboratory Press, Cold Spring Harbor, N.Y., N.Y.
120. Saqi et al. (1999) Bioinformatics 15:521-522.
121. Sauer (1998) Methods 14(4):381-392.
122. Schmidhauser & Helinski (1985) J Bacteriol 164:446-455.
123. Schocher et al. (1986) Biotechnology 4:1093-1096.
124. Shimamoto et al. (1989) Nature 338:274-277.
125. Shinshi et al. (1990) Plant Molec Biol 14:357-368.
126. Shoemaker et al. (1995) Crop Science 35:436-446.
127. Silhavy et al. (1984) Experiments with Gene Fusions, Cold Spring Harbor Laboratory, Cold Spring Harbor, N.Y., N.Y.
128. Singh et al. (1989) Biotechniques 7:252-261.
129. Skuzeski et al. (1990) Plant Molec Biol 15:65-79.
130. Smith & Waterman (1981) Adv Appl Math 2:482.
131. Smith & Camper (1973) Crop Science 13:459.
132. Spencer et al. (1990) Theor Appl Genet. 79:625-631.
133. Staskawicz (1995) Science 268:661-667.
134. Stoneking et al. (1991) Am J Hum Genet. 48(2):370-82.
135. Thompson et al. (1987) EMBO J. 6:2519-2523.
136. Tijssen (1993) in Laboratory Techniques in Biochemistry and Molecular Biology-Hybridization with Nucleic Acid Probes, part 1 chapter 2, Elsevier, New York, N.Y.
137. Trask (1991) Trends Genet. 7:149-154.
138. Uknes et al. (1992) The Plant Cell 4:645-656.
139. Uknes et al. (1993) The Plant Cell 5:159-169.
140. Unger et al. (1989) Plant Molec Biol 13:411-418.
141. U.S. Pat. No. 4,196,265
142. U.S. Pat. No. 4,554,101
143. U.S. Pat. No. 4,940,935
144. U.S. Pat. No. 4,945,050
145. U.S. Pat. No. 5,036,006
146. U.S. Pat. No. 5,100,792
147. U.S. Pat. No. 5,188,642
148. U.S. Pat. No. 5,260,203
149. U.S. Pat. No. 5,523,311
150. U.S. Pat. No. 5,591,616
151. U.S. Pat. No. 5,614,395
152. U.S. Pat. No. 5,629,158
153. U.S. Pat. No. 5,639,949
154. U.S. Pat. No. 5,834,228
155. U.S. Pat. No. 5,872,011
156. U.S. Pat. No. 5,948,953
157. U.S. Pat. No. 5,952,546
158. U.S. Pat. No. 5,958,624
159. U.S. Pat. No. 5,986,173
160. U.S. Pat. No. 5,994,526
161. U.S. Pat. No. 5,994,527
162. U.S. Pat. No. 6,096,555
163. U.S. Pat. No. 6,162,967
164. U.S. Pat. No. RE36,449
165. van den Broeck et al. (1985) Nature 313:358-363.
166. Vasil et al. (1992) Biotechnology 10:667-674.
167. Vasil et al. (1993) Biotechnology 11:1553-1558.
168. Vidal et al. (1996) Proc Natl Acad Sci USA 93(19):10315-10320.
169. Vos et al. (1995) Nucleic Acids Research 23:4407-4414.
170. Wang et al. (1998) Science 280(5366):1077-82.
171. Warner et al. (1993) Plant J 3:191-201.
172. Webb et al. (1995) Theor Appl Genet. 91:574-581.
173. Weeks et al. (1993) Plant Physiol 102:1077-1084.
174. Weiseman et al. (1992) Theor Appl Genet. 85:136-138
175. White et al. (1990) Nucl Acids Res 18:1062.
176. WO 93/07278
177. WO 93/21335
178. WO 94/00977
179. WO 97/47763
180. Worrall et al. (1998) Anal Biochem 70:750-756.
181. Wrather et al. (1995) Plant Disease 79:1076-1079.
182. Xu et al. (1993) Plant Molec Biol 22:573-588.
183. Yuan et al. (1999) Hum Mutat 14(5):440-446.
184. Zhang et al. (1988) Plant Cell Rep 7:379-384.
185. Zhang et al. (1994) Mol Gen Genet. 244:613-621.
186. Zhu et al. (1996) Mol Gen Genet. 252:483-488.
187. Zimmer et al. (1993) Peptides pp. 393B394, ESCOM Science Publishers, B. V.
188. Zobrist et al. (2000) Soybean Genet Newslett 27:10-15.
189. U.S. Pat. No. 7,154,021
190. U.S. Pat. No. 6,300,541
191. U.S. Pat. No. 7,288,386
192. U.S. Pat. No. 7,902,337.
193. Afzal A J (2007). PhD Dissertation. Southern Illinois University, Carbondale
194. Afzal A J, Lightfoot, D A (2007) Protein Expr Purif 53: 346-355
195. Afzal A J, Wood A J, Lightfoot D A (2008) Molec Plant Microb Interact 21:507-517
196. Afzal A J, et al 2008. Nature Preceedings hd1:10101/npre.2008.2726.1
197. Afzal A J, et al., (2009) Plant Physiology 151: 1264-1280
198. Afzal A J, et al 2011. Theor Appl Genet. 2011, doi
199. Srour A, et al. 2012 BMC Genomics (in review)
200. Srour A, et al. 2012 BMC Plant Biology (in review)
201. Concibido et al 2004 Crop Science 44:1121-1131
202. Sequence Listing and Figures Are Shown as Appendices

It will be understood that various details of the invention can be changed without departing from the scope of the invention. Furthermore, the foregoing description is for the purpose of illustration only, and not for the purpose of limitation—the invention being defined by the claims.

Sequence Listing

SEQ NO. 1 > Sequence of the mRNA coding region of the Rfs2?Rhg1 RFK gene encoded by
pSBHB94: gi|300519109|gb|AF506517.2| Glycine max receptor-like kinase RHG1 mRNA,
complete cds
CACACACACTCACACACACTGTTTTTTTGTTCCACTAAATCAAAACCTCTTATCTCTTACTCTCATTACA
TTCATTCTTTTGATTTTCGTTATGGTAGTAGCAGTGGAGAAAACCAACCTCACTTCACAATCACAATGCT
TCAACCGTGTTTCTGACAAGAAGAAAGAAAGATGCAAGACACACATGAACAACGTTAACCCATGTTGTTT
TTTGTTTCTCTTATGTGTGTGGAGCCTTGTTGTGCTCCCCTCATGCGTGAGGCCAGTTTTGTGTGAAGAT
GAAGGTTGGGATGGAGTGGTTGTGACAGCATCAAACCTCTTAGCACTTGAAGCTTTCAAGCAAGAGTTGG
CTGATCCAGAAGGGTTCTTGCGGAGCTGGAATGACAGTGGCTATGGAGCTTGTTCCGGAGGTTGGGTTGG
AATCAAGTGTGCTCAGGGACAGGTTATTGTGATCCAGCTTCCTTGGAAGGGTTTGAGGGGTCGAATCACC
GACAAAATTGGCCAACTTCAAGGCCTCAGGAAGCTTAGTCTTCATGATAACCAAATTGGTGGTTCAATCC
CTTCAACTTTGGGACTTCTTCCCAACCTTAGAGGGGTTCAGTTATTCAACAATAGGCTTACAGGTTCCAT
ACCTCTTTCTTTAGGTTTCTGCCCTTTGCTTCAGTCTCTTGACCTCAGCAACAACTTGCTCACAGGAGCA
ATCCCTTATAGTCTTGCTAATTCCACTAAGCTTTATTGGCTTAACTTGAGTTTCAACTCCTTCTCTGGTC
CTTTACCAGCTAGCCTAACTCACTCATTTTCTCTCACTTTTCTTTCTCTTCAAAATAACAATCTTTCTGG
CTCCCTTCCTAACTCTTGGGGTGGGAATTCCAAGAATGGCTTCTTTAGGCTTCAAAATTTGATCCTAGAT
CATAACTTTTTCACTGGTGACGTTCCTGCTTCTTTGGGTAGCTTAAGAGAGCTCAATGAGATTTCCCTTA
GTCATAATAAGTTTAGTGGAGCTATACCAAATGAAATAGGAACCCTTTCTAGGCTTAAGACACTTGACAT
TTCTAATAATGCCTTGAATGGGAACTTGCCTGCTACCCTCTCTAATTTATCCTCACTTACACTGCTGAAT
GCAGAGAACAACCTCCTTGACAATCAAATCCCTCAAAGTTTAGGTAGATTGCGTAATCTTTCTGTTCTGA
TTTTGAGTAGAAACCAATTTAGTGGACATATTCCTTCAAGCATTGCAAACATTTCCTCGCTTAGGCAGCT
TGATTTGTCACTGAATAATTTCAGTGGAGAAATTCCAGTCTCCTTTGACAGTCAGCGCAGTCTAAATCTC
TTCAATGTTTCCTACAATAGCCTCTCAGGTTCTGTCCCCCCTCTGCTTGCCAAGAAATTTAACTCAAGCT
CATTTGTGGGAAATATTCAACTATGTGGGTACAGCCCTTCAACCCCATGTCTTTCCCAAGCTCCATCACA
AGGAGTCATTGCCCCACCTCCTGAAGTGTCAAAACATCACCATCATAGGAAGCTAAGCACCAAAGACATA
ATTCTCATAGTAGCAGGAGTTCTCCTCGTAGTCCTGATTATACTTTGTTGTGTCCTGCTTTTCTGCCTGA
TCAGAAAGAGATCAACATCTAAGGCCGGGAACGGCCAAGCCACCGAGGGTAGAGCGGCCACTATGAGGAC
AGAAAAAGGAGTCCCTCCAGTTGCTGGTGGTGATGTTGAAGCAGGTGGGGAGGCTGGAGGGAAACTAGTC
CATTTTGATGGACCAATGGCTTTTACAGCTGATGATCTCTTGTGTGCAACAGCTGAGATCATGGGAAAGA
GCACCTATGGAACTGTTTATAAGGCTATTTTGGAGGATGGAAGTCAAGTTGCAGTAAAGAGATTGAGGGA
AAAGATCACTAAAGGTCATAGAGAATTTGAATCAGAAGTCAGTGTTCTAGGAAAAATTAGACACCCCAAT
GTTTTGGCTCTGAGGGCCTATTACTTGGGACCCAAAGGGGAAAAGCTTCTGGTTTTTGATTACATGTCTA
AAGGAAGTCTTGCTTCTTTCCTACATGGTGGTGGTGGAACTGAAACATTCATTGATTGGCCAACAAGGAT
GAAAATAGCACAAGACTTGGCCCGTGGCTTGTTCTGCCTTCATTCCCAGGAGAACATCATACATGGGAAC
CTCACATCCAGCAATGTGTTGCTTGATGAGAATACAAATGCTAAAATTGCAGATTTTGGTCTTTCTCGGT
TGATGTCAACTGCTGCTAATTCCAACGTGATAGCTACAGCTGGAGCATTGGGATACCGGGCACCTGAGCT
CTCAAAGCTCAAGAAAGCAAACACTAAAACTGATATCTACAGTCTTGGTGTTATCTTGTTAGAACTCCTA
ACGAGGAAATCACCTGGGGTGTCTATGAATGGACTAGATTTGCCTCAGTGGGTTGCCTCAGTTGTCAAAG
AGGAGTGGACAAATGAGGTTTTTGATGCAGACTTGATGAGAGATGCATCCACAGTTGGCGACGAGTTGCT
AAACACGTTGAAGCTCGCTTTGCACTGTGTTGATCCTTCTCCATCAGCACGACCAGAAGTTCATCAAGTT
CTCCAGCAGCTGGAAGAGATTAGACCAGAGAGATCAGTCACAGCCAGTCCCGGGGACGATATCGTATAG
SEQ NO. 2 >Glycine max cultivar Peking RFS2/RHG1 receptor-like kinase (Rhg1/Rfs2)
gene, Rhg1/Rfs2-Forrest allele, complete cds 9772 bp DNA linear PLN 30-
NOV-2011 HQ008939.1 GI: 330722945
AAGATAAAATTGCTAATTATTGGTTAAGAAAATAATTGCACCAGATATATTATATAAAAT
GTCAAAAACGCATTCCGTACATTATAAATAATATTATATACGTCATATTTACATCATTTT
TTATCCTTGTTTATCTCAAAAAAGTGTAAATATAGAGAGAGTATATATCATATCATATAA
TATGTAAGTTTTTATTAGTTTaaaaaaaTAGCTTGAGAGTAATGTGATTTGTCATGTGCT
AATAAAATATCATTTTGAATGCTCTTTTATCCACATATATTAATTGTTAATGATTGAAGT
TTATTATTATTATTATAATATCCTTTTAACGATGAAAGTTTGTTTTaaaaaaaTATAGAT
TTAAGATGTGTTTGGAGGAATTTATTTATATCTTATCTGAACTTATTTTATGGCATACGT
GTAAGTATTTAAGAAAACTTATAAAATTATAGTTTATGATTTATTTATAAATTGTTTTCA
ACttattttaataaaattttcaaaataacttataagaacaaattaaattttttatatgaa
aataatttaaccttattttcttttcaattataaaaaacaatttaCAAATAAAAGCTTATA
TATATGATACACACTTTTAAGTGTTTAAGTAAGCTATCTAAAAAAGGCCGTACAGTGTTT
CTTTAATGAACTATCGATCGGGAATGTTATATATGGAAATATATATACTTGAGTGAATAT
AGGCTCGATTACTCCATAGTACAGTCCAATAATTATTAGTAAACGAATTATACGTTTAAT
TTGTATCTATATATCTTTTGTTGATAATTGATGTAATTTCAATTTTAATTTACCAAAGAG
AGTTAGCACCACAGCGAGCATCCGTTGCCTCATTAGTCATTAGTACTTATCACCGACATC
TTTTTGTTTGTAAAAGGACCACTGATTCATTTACCTACATATATAATATACAATATGTAT
GTATACAAAAATCATAGTAAGGTTTAAATGTAATGCTTCATGAATAAGATATTCTGTGTT
ACAGATTAAGATTCGTGTATGATAAAATGTTTGTTATTATTAGAGTTAACCGGCAATTTG
TTCATATTGAGTCTCATTAATTACCTTCTTTTCACATGTTTTGTTGACATCGAGAGTGAC
GATCCTACCGAGATAGATAAGGATATATATGATAACAAATTGAGATAAAAAGCTCTTTGC
ACAGTCAATTATGATTAAGAAAAATATCAAATCAGTTTTACAGACCGTAGCTCATTAGGC
AGAGATAATTACATGCACGTAAAGAAAAAATTATTGAGTCACTAAAATTGGGATAGCGAG
GAATTTGAGTAATTTGAACTAAGTCATAAGTTTAAATCGTATCGTTaaaaaaaaTGTAGT
TTTTGTTACTCTTTTAAATACTAGTAtttttttttttGAAAGGTTTTAAATACCAGTATT
ATTCCACTAATAACCTGCCTTTATTTCTTTATATAAAGCCTTCTCTTAATGAAAATAGAA
TACTAATTAAATAATCGAGAGAAAAAAGATACAAATGGAGAACAAATTATCATGAAAAAG
TTACACATTAGAAAATATACATGTTTTAGCATTGAAAAATACAATGGTCAATTATAAACC
AAAGAGGCCCTTAGTTAGTTAGTCTAATGTTTAAGCCACCAAATTTTTGGTTGATAACGT
TTAAAAGTAATAGCTAGATGGTCTCTTTCAAAGAAATTTCTGTCCATATTATTCAGGTTT
CAAATTTTGTTTGTAAGACGAGGAATTTTGGATCTTGATGATAAGAACAAGACAGGGTGA
ATAAGTTCATTTAATTAAGATGGAAAGTGCGAGTTTAACTTGAGTTACGTGTAAGGTTTC
ATAATCAAGTGTACATATGTATATGTATTAGGGTAGATTAATGATATTAGCTATCAAATT
TAATAAAATGTATATTTAATATTAtttttttATCAACAGTAAATTTTGTTAATTTAACAG
TTGAATTTAAAGTTTTCATAAAATAAATTAAACCCCACATTATTTCAAAAAGTAATTAAT
ACTTTGTTACTACACTCTTAATTATATGCATAATGCATTATATTTTGTAATAAAAACTTT
ATATTTACACACGTATGACCATTGGTGAACCTACACTGTGGCAAGTACACCCTCATTTTC
TAACATTCACAAATAAAAGtttttctaaacagaaaattataataaaatcttataatttta
tattttatttcatttatatttatatatttatgataaattcctatatttatatatttaaac
ccactcattttactttttataatttattCACATTGATTCAAGTTCTAAATCTACACCCAT
CGAGTGCATAAATCAACTGGCATATATTTTAACTTAATCAAAGGTCTTGAGTTTAAGTTT
TGAATATAAAATTACCTTATATATTTAAAGGAAGAGTTTGTTATCCATGATGATTCCATA
AGACTCTCTAACAAAATTACTTCCAATAAAATATACATGTGGTTTATaaaaaaaaaattc
catcaaaattttacaaaaacaatacaaaaagaataaaaatatttttttaaaaaattaatt
catttattttGAATACATTACTTACTTTtatatatatatatCAACAGGGACATAGTAATT
CAAGACTATTAATGTTGTTCACCCGTGACACATGTCAACTCAATATTACACAATcattat
caaatttaattttagaaaatttaatattttttcccattagcatatagtcatttttattGG
AAAATACATTGATGAAACATATTATACTAATTAAAGGATAAACATTATAATTTATAAAAG
CATTCAACTATATCCATTAATTGTAAAGAAAATTTTCAATTGAGAATCGAAGTTAATAAT
TATCAAAATAATTCTTGCTTTTATTTATGAAAATATATTGTGTGATTCTTAATTATTTTC
ATAAATATATAAAAATGAATATCATCATATATTTTGAAGTAACTTAAAATATATTTAATC
CTAAGGTTCTACATGCTTGAACAAACGTCTTCATCACAAATCTTTGTAGAAAAAGTAAAT
AAGACACTACCaaaaaaaaaaaaaaTCACCACCACTACAAATAAAAAAGGTACGCAAAAA
GAGAGCTTACACTATTACCACCCTACACACTGTCTTTTATCCACATATTCCTTCTCAATC
GGTAAAAGAACCAATAGCTATGATAGACATCCCCGGCCGGACTCGATATTTTTTCAAATG
TTCCCTCAAATCACTGTTAGTTTTGATGTTAAAACAATTTGTTTCTTGGTTTTGCTAGTG
AACCGCTTGATTTCATATAGCAAAATAAGTTCCtttttttttttttttGTAGGCTAGAAA
AATAAGTTGCAGTAGATAAAAATAAAGACAAAGCATTCTGATCGCTATAATTGTAACCAA
TGTGCAATATTAAAGGGGTGTCTGAGAGCATACAATATCATTTTGTAGCCTTTTATACCC
ATTTCACTTAATTTGCCCATGTTCTCTGTCCACTCGTTTGATGTCTTCTAAGTAATAACT
ATCAGTTTCATTGACCTTGTGGTCATAACTCATAACTACCATCCTTGAGCTAACACAAAG
AATAAAGAGATATTTAGGAAGATAAAATTGTGCGAAAGTAAGAAACATTCAATTGTAATA
TGCTTCAACAATAGTATGGCCAACAGTAGTGGCGAATCTAAGACTCTGACTAAGCAGCCA
TAAATTAAAGAAGCTTATTTACAACTAGTGTTATCGGAGAATGAAAAATTGAAGAATAAT
AAGTTCAGCTATAATAAACTCGAGGGAGGAAAAACAAAGAAATTCATGATAAATAGATAT
AACTTATTAAATTTAAGGGGTGTATTTGCACACCCTGAATTATAGAGATTCTTATATCTT
TGAGAAAATAATTAAATTGGGAAAAAAGAGATAATGACTGATTGAGATTTGCCTCAGAAT
TGTTCGTTTTAATATTGGTACGAATCTAATGGTTTTATCCTGAAAGATGCTCACAAGTAT
TGAGGGACTAATAAATTGTTTATAAACTACTACTAAATGAGATGAGACTTTAAGGTGTAC
TGAAGCAATATCATTTAAAAAATGACTACTCGTATTTGTGTTGAGAAAATTTATTTTCAA
TGAAAAGAAAATATATACATATAAGATAAAGTAATTAACATAACGAAAGGAAATAAAATG
CAACATTATAAAAACTACAACTATATAAATGATATATACAACTCCTAGCACATGCATTGG
ATTGTGAATTAATTAAAATGTTGTATGGATGGTAAAAATTCAAAACTAAACCCCACACAA
TTTAGTGACACAGAATATAATTAGCGTTGTTCTTTTTACAGAAAACGACGAGAACAAAGG
TGTCAAAGGAAAGGAGATGGATGCATGTGGTATGAGCTCATCCAATTCCAAACATGTTGT
GGACCAAAAGCGAAGTACCATGAACATGATGATCACGACGATTCTTCTCAGATTTTGGGA
CCGCTATGATATGAATTGCGACTACACTACTAACTCTTACGAACCGGGGTCATCATAAAA
CCATTACCATTTACCACTCTTTTGAACGTTAATGTAGCCTAAATCTTATATCCAGAGAAC
CAGACCCTGTTTACATTTCCTTTTTAAAACGTTTCTGATAAATTTCTCTTGCTAGTGTCT
CAGAACCCAGTTAGCTCCTTCCTCACCACGTGACACTTCAGTGAAACTTGGAGATGCCAG
CAGGTTTATTTCAGCCAGGGTCTTTGTCTCTCAGGGCAATTCATTAATTTAAAAAATAAC
AtttttttATACATATTCATCAGTGCACGAGGAGGAGGGATAGTATGTATCACACTTTTT
AATTCACTTTCTATTGTTTTCTGTTAGTTGAAATTCAAATATCCCTCACTAATTTGAGAC
TGAAACATTTCACCaaaaaaaaaaaTTGAGGATGGAACTTTCTTTTTTAGTTGATCATAA
ATTTTTTCTTCTAAAATATATAATGTGGATACATATTTTTTGAGATTGAAACCTAACAAA
TGATAAATAAGACTCACTTATTTAGTGAGACATACATGAATTTCAGAGAATATTTTCCTA
TATAGGTTATTAGCATTTCTTTTAATAtttttttttattgtcttgtttttaaaaagttgg
cattctttttaaaattgacttttttgAGATATTGAACTATTttaataataataataaaat
taagttatatagtgtattaaaaagaataagataaaatgtgttttaaatTTCTCAAGATTT
TAGTCAAAATTAGTTTCAGTCTCCTCTATTAAAAATGTGTTTTAATTCTCATATTTTTAA
AGATATGGTGAATTTCATTTTTAATCTTGAACAGTTCTTTAATTTTGACTTAATTAAATT
CAACATATTTCAGAAACACGGGAACCAAAACCACCATTTTTAGAATCCAAGACTAAAGAT
CTTAATGACGTAAAACACAATTTACCCGTGAGAATATTAAAGCTAGTAGTATTGCTTTTC
AGTGTGTTTCCTACGGCACATTGTTGTGTGTGGAAGTGGAAGCTAGAAAACAAAGGCAGC
AGAAGAAGTATGGTCCTACAAAGTGTGTAGTAGTGAAGAAGAAATAGCCGTTGGTGGTGG
AGAGGCGCGGGTTTGCAATAAAAGAACAGCGCGCCATGATCCTATAATAAACCCTGTCAA
CAAAAACAAGTATGCTTCATGAATAGTTACTATTTACAAGGAAAACTAGCCGTTACTCAC
TTTTTCTTCttttttttttttttGTAACAAATTCTGAACCCTGCATGTTCATtctctctc
tctcACGCTCGCAACCCGCGCGCGCACCTACACTTCTTTTATGTCATCACGTGCTCCTTC
TCACTCTCCCTCTCTCTCACTACAAAAACCATTCTTCAACTTGCAacacacgcacacaca
cactcacacacacTGtttttttGTTCCACTAAATCAAAACCTCTTATCTCTTACTCTCAT
TACATTCATTCTTTTGATTTTCGTTATGGTAGTAGCAGTGGAGAAAACCAACCTCACTTC
ACAATCACAATGCTTCAACCGTGTTTCTGACAAGAAGAAAGAAAGATGCAAGACACACAT
GAACAACGTTAACCCATGTTGTTTTTTGTTTCTCTTATGTGTGTGGAGCCTTGTTGTGCT
CCCCTCATGCGTGAGGCCAGTTTTGTGTGAAGATGAAGGTTGGGATGGAGTGGTTGTGAC
AGCATCAAACCTCTTAGCACTTGAAGCTTTCAAGCAAGAGTTGGCTGATCCAGAAGGGTT
CTTGCGGAGCTGGAATGACAGTGGCTATGGAGCTTGTTCCGGAGGTTGGGTTGGAATCAA
GTGTGCTCAGGGACAGGTTATTGTGATCCAGCTTCCTTGGAAGGGTTTGAGGGGTCGAAT
CACCGACAAAATTGGCCAACTTCAAGGCCTCAGGAAGCTTAGTCTTCATGATAACCAAAT
TGGTGGTTCAATCCCTTCAACTTTGGGACTTCTTCCCAACCTTAGAGGGGTTCAGTTATT
CAACAATAGGCTTACAGGTTCCATACCTCTTTCTTTAGGTTTCTGCCCTTTGCTTCAGTC
TCTTGACCTCAGCAACAACTTGCTCACAGGAGCAATCCCTTATAGTCTTGCTAATTCCAC
TAAGCTTTATTGGCTTAACTTGAGTTTCAACTCCTTCTCTGGTCCTTTACCAGCTAGCCT
AACTCACTCATTTTCTCTCACTTTTCTTTCTCTTCAAAATAACAATCTTTCTGGCTCCCT
TCCTAACTCTTGGGGTGGGAATTCCAAGAATGGCTTCTTTAGGCTTCAAAATTTGATCCT
AGATCATAACTTTTTCACTGGTGACGTTCCTGCTTCTTTGGGTAGCTTAAGAGAGCTCAA
TGAGATTTCCCTTAGTCATAATAAGTTTAGTGGAGCTATACCAAATGAAATAGGAACCCT
TTCTAGGCTTAAGACACTTGACATTTCTAATAATGCCTTGAATGGGAACTTGCCTGCTAC
CCTCTCTAATTTATCCTCACTTACACTGCTGAATGCAGAGAACAACCTCCTTGACAATCA
AATCCCTCAAAGTTTAGGTAGATTGCGTAATCTTTCTGTTCTGATTTTGAGTAGAAACCA
ATTTAGTGGACATATTCCTTCAAGCATTGCAAACATTTCCTCGCTTAGGCAGCTTGATTT
GTCACTGAATAATTTCAGTGGAGAAATTCCAGTCTCCTTTGACAGTCAGCGCAGTCTAAA
TCTCTTCAATGTTTCCTACAATAGCCTCTCAGGTTCTGTCCCCCCTCTGCTTGCCAAGAA
ATTTAACTCAAGCTCATTTGTGGGAAATATTCAACTATGTGGGTACAGCCCTTCAACCCC
ATGTCTTTCCCAAGCTCCATCACAAGGAGTCATTGCCCCACCTCCTGAAGTGTCAAAACA
TCACCATCATAGGAAGCTAAGCACCAAAGACATAATTCTCATAGTAGCAGGAGTTCTCCT
CGTAGTCCTGATTATACTTTGTTGTGTCCTGCTTTTCTGCCTGATCAGAAAGAGATCAAC
ATCTAAGGCCGGGAACGGCCAAGCCACCGAGGGTAGAGCGGCCACTATGAGGACAGAAAA
AGGAGTCCCTCCAGTTGCTGGTGGTGATGTTGAAGCAGGTGGGGAGGCTGGAGGGAAACT
AGTCCATTTTGATGGACCAATGGCTTTTACAGCTGATGATCTCTTGTGTGCAACAGCTGA
GATCATGGGAAAGAGCACCTATGGAACTGTTTATAAGGCTATTTTGGAGGATGGAAGTCA
AGTTGCAGTAAAGAGATTGAGGGAAAAGATCACTAAAGGTCATAGAGAATTTGAATCAGA
AGTCAGTGTTCTAGGAAAAATTAGACACCCCAATGTTTTGGCTCTGAGGGCCTATTACTT
GGGACCCAAAGGGGAAAAGCTTCTGGTTTTTGATTACATGTCTAAAGGAAGTCTTGCTTC
TTTCCTACATGGTAAGTTTCGTGTGCTGTTCTTTCATTAAGTGTTGTGTGTGCTGTTCTT
TAATTATAATTTGGAGTTTTACCTTAGTAATCTGTATAATTCTAATCGGAGAACAGTACA
AACAAAAACACCTAAGGAACAACACCTTAGCTTTAATATACCATATCAATAAGTGAATTA
TTTTCTTGTTCATCTTGATGCAGGTGGTGGAACTGAAACATTCATTGATTGGCCAACAAG
GATGAAAATAGCACAAGACTTGGCCCGTGGCTTGTTCTGCCTTCATTCCCAGGAGAACAT
CATACATGGGAACCTCACATCCAGCAATGTGTTGCTTGATGAGAATACAAATGCTAAAAT
TGCAGATTTTGGTCTTTCTCGGTTGATGTCAACTGCTGCTAATTCCAACGTGATAGCTAC
AGCTGGAGCATTGGGATACCGGGCACCTGAGCTCTCAAAGCTCAAGAAAGCAAACACTAA
AACTGATATCTACAGTCTTGGTGTTATCTTGTTAGAACTCCTAACGAGGAAATCACCTGG
GGTGTCTATGAATGGACTAGATTTGCCTCAGTGGGTTGCCTCAGTTGTCAAAGAGGAGTG
GACAAATGAGGTTTTTGATGCAGACTTGATGAGAGATGCATCCACAGTTGGCGACGAGTT
GCTAAACACGTTGAAGCTCGCTTTGCACTGTGTTGATCCTTCTCCATCAGCACGACCAGA
AGTTCATCAAGTTCTCCAGCAGCTGGAAGAGATTAGACCAGAGAGATCAGTCACAGCCAG
TCCCGGGGACGATATCGTATAGCACAAATTTTGCATTGAtttttttGTGCCAAATGTAGT
AGGCCTACTATATATATGTTCTATGATTCTTTCATTCTTATATTATTTTTGCCTGTTTGA
ATGCTTGAATTTGTACATACTCATACTACAATAAGGTGTAGTTCTGGTTAATTTTACCTC
TACCTCAAAGCTGGGGTGTAATTCTGTTTCCTCCAAGGCACATAATAGTTGAAAATAGTT
CTCAGGAGCATTCATTGTTTATTCTGCAAGATTCTCTTTCACGGCTGCTATCTTCTATGC
ATGCCCTGCCCATAAATGCATTATGAAGAATTGTAACGGCTGTGTTTTTGGACTTCTTCA
AAAAGTTTATGTTATTGCCAGGTGTATATATCAACATGTTTTAAAGATTTTCAAACAATC
AGGTTTTAGATGTGGGTTTGCATGCATGAGATTGGACTAGTGCGCTTGATGTAGTATAAA
ATATAAATTGTCCAATCAGCACCCTCTACATGTCCAAATAATGGGCCTTATGAAACTTAA
TTTTTTAATTACAAACTACAGTAATCTTTTTGAATAAAGATTTACAAATTACAACAGACA
TGTGAAGTCGTCATCTTTCATTGCCAATTCTTTCAAGTTTACTACTATTATTTTCCTGCA
AGCATTCCACATTCACATCTGATAACTATGACAGCATCTCCCAAGATAATGACTTCCAAG
TTCCAACACTGGCTCTGTACATTTGAACTAATTTTATATCATTTATCTATTGTGATTGAA
ATATAAAATTGAAGTGATGTGAACAATACGAATCACATCTTGAATTAAAATATCTAACAA
CGGGAACaaataagaggcccagaaaaaagggataaataacggataacaagaaagaaagaa
aaaaaaaCCCAACATAATTCCAACTTCAAAATTCACTCAATAAAAAGTTTAACATGTAAA
TTTACTTGGAAACAAAACTCATAAGCAAAGAAAGTCAAAGTATACATAACCA
SEQ NO. 3 Sequence of the Receptor Like Kinase encoded by pSBHB94
MVVAVEKTNLTSQSQCFNRVSDKKKERCKTHMNNVNPCCFLFLL
CVWSLVVLPSCVRPVLCEDEGWDGVVVTASNLLALEAFKQELADPEGFLRSWNDSGYG
ACSGGWVGIKCAQGQVIVIQLPWKGLRGRITDKIGQLQGLRKLSLHDNQIGGSIPSTL
GLLPNLRGVQLFNNRLTGSIPLSLGFCPLLQSLDLSNNLLTGAIPYSLANSTKLYWLN
LSFNSFSGPLPASLTHSFSLTFLSLQNNNLSGSLPNSWGGNSKNGFFRLQNLILDHNF
FTGDVPASLGSLRELNEISLSHNKFSGAIPNEIGTLSRLKTLDISNNALNGNLPATLS
NLSSLTLLNAENNLLDNQIPQSLGRLRNLSVLILSRNQFSGHIPSSIANISSLRQLDL
SLNNFSGEIPVSFDSQRSLNLFNVSYNSLSGSVPPLLAKKFNSSSFVGNIQLCGYSPS
TPCLSQAPSQGVIAPPPEVSKHHHHRKLSTKDIILIVAGVLLVVLIILCCVLLFCLIR
KRSTSKAGNGQATEGRAATMRTEKGVPPVAGGDVEAGGEAGGKLVHFDGPMAFTADDL
LCATAEIMGKSTYGTVYKAILEDGSQVAVKRLREKITKGHREFESEVSVLGKIRHPNV
LALRAYYLGPKGEKLLVFDYMSKGSLASFLHGGGTETFIDWPTRMKIAQDLARGLFCL
HSQENIIHGNLTSSNVLLDENTNAKIADFGLSRLMSTAANSNVIATAGALGYRAPELS
KLKKANTKTDIYSLGVILLELLTRKSPGVSMNGLDLPQWVASVVKEEWTNEVFDADLM
RDASTVGDELLNTLKLALHCVDPSPSARPEVHQVLQQLEEIRPERSVTASPGDDIV
SEQ4>BAC B73P6 complete sequence from cultivar Forrest. Glycine max clone pCLD04541
Rhg1 gene locus, complete sequence GenBank: 82157 bp DNA linear PLN 22-
NOV-2011 JN597009.1 GI: 357432827
gi|357432827|gb|JN597009.1| Glycine max clone pCLD04541 Rhg1 gene locus, complete
sequence
TGCACAATAGTTATGAACGTTCAAAGTCTGTCCAACCCAACTTTGCATGATATTCAACCTTACGGGCATG
TAATGAGGAAATCCCCTTAATTTTACTAAAATAAAAATCAAAACAAGGATAAAAATAAAATACAGATAAA
ATCCTTAAGCTACAAGTCTAATCAGAAAGAAAAAAAATATGATCCAGAATCATCAAATGTTTACAATTCC
AATTAATCTCTTTGTAACACGTAATGTTAATTCTTTTCGTAAATTAAAAAAATTCAACTACATGTTGTGT
AAATTTACAAAAATTATATACACATAACAATCTCAAATCAAAAAATAATTTCTGAATGCTCATCCACAGA
AAGGTTTAAACTGCACGTCTTCACAAGGTGGATTCCTTAATACAGTCAGTGTATATATATAAACCTAAAA
GTAATTTATTAGAAGGTGCTAATAAGTTGGTGAAGCAAGCCAATCTTTCCATAAAGCAATGGTCATGGAA
GGTGATACTTGGAAAGAAAGTACCTGAATTTCTTGGTAAAGCAAGAAATTATGTAAACAAAGACATTGGC
GTCAGGAAACAGCATCTCCATTTTAAGTGCCAATTTATGGTGCGTATTCAAACATCATGTCAAATAAAAG
AAAGAAATTCTTACGACTTTGTACCATTTCATGCTGTAATTGAGGGGTACACATTTTTTTTCCAACAACT
TGAGGGGTACACGTTGAACAGAACCTAAACCGTTCTCGTCGAATAATACCGATTCGACAAATAAAAAATG
AATAAATTATATTGGCAAAACAAAAAATAGAATAAATTATACTTTATTTTCCAACTATTTCTTACTTTTT
TAGTTTTCTCTCTCTCTCTCTATAAGTTATATATTTATATACAAAAAGACGAAATTCGTAAGGCAATCTT
ATTGGTATTTTAATTTTCTCTACTGATTATGTCTAACCATTTATACACACACACACATATATATATTGTA
TTACTTGTTAAATAAAATCAGAAAAATGTTGTAATCACTTTCAAAACTGTAGTTAATAAACCTTAACTAA
ATCAAGCAAAAACAATGGATAAGATGGAAGTTTAGTGATACAAAAATATATACAGGTATAGTGAGAATAA
AAAAGTATAAAAAAAAGTTGAGGAAGTGTGAAATCTACGTGAAGATGAAGGATGAAAATTGGTTAAGTTA
GGTTAGGTTAGATTTTATAAAATATGTATGAGTTTGATTTGTTTTTTAAGGTTCGAGCTTAGTTTATATA
TTTAGTTGATATAGACTACATTTAAAAGACTGACTTAAAAGTCTCTTTAAAATACATAATAATAACGAAA
ATGGATTAAGTTAGATTTGGTTTTTTTTTTTCTTTTTACTGGGTAGTTAGATTAGGTTAATCTTTATAAA
ACATGAATTTGATTTGTTTTTTTTTTCAAAAAAAATTAAGGTTCAAGCTTAATTTATTTAGTTGATATAA
CCACTTTCAAAAATCTGACTTACAAGACTCTTTAGAATTCATAATAGTGACACTTGATTAAGTTAGATTA
GACTTTATAAAACACGAGTTTGATTTTTTTTTTAATAATAATTAAGGTTCTAGCTTATATATATTATATA
GTTGATATAGACTACTTTCAAAAGTCTGACTTAAAAGTCTCTTTAGTATACATAATAATATAACCTTTTA
ATTTAGTTAAAAAATTTGTCCCTAAATAAATTAATAAATCCAAACTTATATACAAGTTAATAGGCTTAAG
TCTTAAAAAAATAATATATATATATATATATAAAGCATTAAAACATTTCAATGAAAACAATATAATAATA
ATAATAATAAATATATTATTGTTATTAATTCATAGATTTTATTATTACTATTATAGAATAATTTGTGTGT
ATATATATAAATATATAGAGAGAGAGAGGGTCATTTTATATGAGTGAGAAAATTTAAATATTATTATGAA
TTTTCAAAATTAAAATACACATGCCATATGATTTTCTTAAAAAATTACGTAACTTTTTTTTTTACAAAAG
TAATCATATGGTTTTAAAAACTAATTTAAATAACTTATATATAACTATATCAGTTAAATTTGGTTCATAA
AATAAGTATATCAGTTATTTTACAAAATTATAAGTATTCATAAAATAAATACAAAATGATAAGTACCAAG
TGTATGGATCAGCTTATGCGATGTTGTTCCAATGTAACTAATAATCTTAAATTCGAGTATTGAATCGAAT
ATGCAATTCATTTAAATACTTTAAGAGATAATTTGTTTGCTCGTAATAATTTTATTTGACTTAAGTAAAG
TTTTCTCATATAAAAAATACATATAGTTATAAAAAAAACATTTTCTGACTAAATATATTTTCACAGGCTC
CACACAAAAAGGAAATAACAAAATTTTAAGAGAATGTATATTTACACTCATCAGTTTGTTAAAGTTAAAA
TTAAAATGGAGTAGAAATTGAGAGAAAAGAGAGGAAATATTTAAAATAAAGGTTGATTTGTATAAATAAA
ATGTGAAGGAAAGAAATAAAAAACTGGTGAGTATTACTCAAAATTATGTTTACAATGATGAAAGGAAAAC
TGATAAAGAAAGTAAACACTAAAAAAAGAGACCAACTTACAACTAACATTTACTTCGAGCATAGTTAATT
AAGTGTTTGGGACATAATATTGTTTCTATAACTAAGTCTTACGTAGATTAAGGTTTACGAGACCTATCCT
AAAAACATGAATTGAAACTAGTATCACCTGTCTTCTGGGCTCAATCCTGGGGATTCATAAAGACATTTCT
TGAACAATCAAAGGGGTTATATGAATGGTTTAACGATCATTAATGTGATATTGATTGACAACCGATCAAT
GCTAGATATATAGGCTTAAAATCCTGTATCAGTCTACAGACGACTAATATGATGTAAAATTCCTTAGTTT
TAAGTGCTTTTGAACATCAAGAGACTTAAAGTTCCGTGTTGGTTGACAAACAAATGGTATGATATATAAT
CCTTCAACGTAAACACGAAAAAAAAAACTTATAATCTCGTGCCAATCATCGATACAGTACAAATAATAAA
TTAAAATGCAATTTTTTTTCTTGTTCTTATTTTTTCTTATTTCTCTTAAACTAGATATACTATCGAATCC
ATTCTATTTCTTATCTATTTCCATTATTCTACTTCTCCCTTATTTTCATTACTTTATTCCTTTCTTTTAT
GTTTCTATCCACTTTATTTATCACCTATTTCTTTCTTTCTTACCGAATACTAAACAAGCCTTGTGATCCG
AAAGCCCGAAACAATCATTTTTTATGAAACAGCTTACACTCTGGTGGTGTGTTGTGTAAAGTTAAATAAG
CTTTTAAAAAAATGGTAAATTATAAGGTGAGGGACCAAAATGTGAGATTGAAAATAACCGTTTCATATAT
TATTCAAATAAATAAATGGCTAAAATTGAATCATTCTCCGTATATAATGACCACCCATTTATTTTATTAA
TATATCTAACAATTATTTTTAACTCCATATAGGCATTTTTGACCCTCATCTTAAAACTCACCTCAAGAAA
TATATAGTTATTTTAATTAAATTAGTACTCAACTTCAAATTAATTATTAGACAAGTGTTGTTTTTTAACC
ATTTATCAAATTAGGAACTTTATGTCACGTTATCCTAAAATCGTTACATAAATTTTTAATGTCACGATAC
AATTTTTTAGAAGAAAAATTTGTCTGAAACCCATATGACATGGGATGCATTAGTCAAAGTAACACTTCCT
AAATCATCAACTTAGTTAGTGGCATGCAACATGGCGTTAACCTATTTTTTTTCTAGAAAAAAAAAAACAT
ATAAATATCACCAGCTGATGTCGCGCCACCTTCAACGCCCAGCCCAGTGTAGGCGCACTATGAAATCAAT
GCAGTCAGTTTTGTCATGTCAGACTTGCAAAAAAATCTCCAAAATTATATCACACTTAATAATTTTTATA
AAGTATTATGCATAATATTTTTTTAAAGTTATTATGCATAGTATCTTTAAAGTTTCATTACCACTATTTT
TCATTAGATAAACTTATTTAATACTTTGTGTAGGGTCATATATAACCAAAAAGTAATCTTGAATGTTAGA
TTTAAATATAAATAAATTTATTTTATCTTATTTTATTTTAATAATTTTTTTATTCATGATGAAAGATATT
GTAAAAATAATTTTATTTTCCCATTGAAATTAAAAAAAAATTAAATGATATGAACCAACCTTTTAATTAA
TATGAAATTAGTTATTTTTATTTATATATAACTTTAGATAACATATTTTTACTCAAAAGACTAAATTAGG
TGGGAATGATGGTCAGATTAAGTGTTGTGGAATTATTGAAGTCTTATATTCAAAGTCCTAACTTATAACT
ATATTAAATACTTAAAAGTTTTTTATCATCTATAACCATCATATTCAATTCTAAAAGAATTGTTTCTAGT
AAAAAAAAATCCGTGACTCAGAAAAAAAACATTACTTGGGTTGATTTTTGTAGCATCTAATATAATTAGG
TGTTGACTCCAATTTTTATTTTGACGGAAGAAAGTTCAGAATGGCGAGAAAGTGTTGAAGCAATATTGGT
TGTCGACTTGTCGTGTCTGAGACGTGTACGTGTACCTTCCTTACCTAAAAAAATGACAATTAAAAAGTGT
TTATTCGGGACGCGCTTAACCAACTTCACAATTTTTTAAGTACCACTCCGGGTGCTATGTTACATGGGCG
TCAGTCTATTTTTTTTTTTCTTTTTAACTAAGAAAAATTAGTATTTTAACCAATTATATAATTGTGAGAT
TAAATATTTAAAATATATAATTAAAATTTATAATAAATAAATATTTTTAAATATATTAATTATATTTGTA
TTTATAATGAATAATTTATATAGTAATATAAAATTATTTTTAGTTATTGATATTAAAAATACAGATGAAA
AAAATATATTGAAAGAGATGAAAGGTTGAGAATTTTTTTAAAAATATTGTTATTGAAAAATTATTAGAAA
AATTCATTGAAGAATAGTTTTTAAAAAATTCTCATTCAAGATAATTATTCTTATATATATATATAAATTT
ATAAATGATTTCATGTAATACGAAAAATTGTTAATCCACATGACAATCTTAGCGAAGTGGCAAAACTGCC
TTGGTAAAAAAATTTCATAAGATAATCCCTTTTAAATAATTAATTTGTAATTAATATTGTGCCATCCTCG
TTACCTTCTGTTCATTTAGCTTTAAGAACAAATTTATGTTTCGCATCACGTAACGTGTGTGTTTGGAAGC
GTTGAAACCACATTCAATGAAAGAAAAAAAAAAACATATTCTAAGACACAAGCAACAAAAGGAAGGTTTC
TTTAACGTCAGTTAGGTTGAGATAAATGCGTACGCAAAAACAGTTAAATGATTAGCTTTTAGAAAAAGTT
AGAGGTTATAAGAAAACATAGGGAAATGTCACTAATGTGTTATACTTTCAATAATCAAGATTTTAGTTTT
CTCTTTCAAAAACATATTAACTTGTTCTCTAATTACCATTTTTAATTTTTAACTACATTTATTATAAATA
CTTTAGTGAAAAACTCATAGCAGCTATTCCAGTTCTGTTCATGTAAATATCGTAGAAGATAATTGCATTT
TTTTCCTTTTCTTTTTCTAAAACAAGAAACGTGTGAGGAATCTTAAGAATTAAGATGCTAATTTAAAAAG
TTGCTGAGTTAGAGCATAAAAGTTCAAATAAAAAATAAATGAATAGACAAACTATTAAATTATTAATAGC
CTTAGCCTTGAATCTGATGCAGACGTGTATGGCAATGGACAGAGAAGCATTAAATAGGCCTCGTTACATT
CAAGTTTCAACCAAATTGACAGGGAAAATCCTCTGATACTGTTGTTTTCCTGAAACCATCACAATTTGTT
TCTCAATCATGTCAACCTCATCCTCTTCCCAAAGCCTCAAAATTGGCATAGTTGGATTCGGCAACTTTGG
CCAGTTTCTGGCCAAGACAATGATAAAACAAGGCCACACTCTCACAGCAACTTCTCGATCTGATTACTCT
GAACTTTGTCTCCAAATGGGCATCCATTTTTTCAGGTAAGTCAAACCAAACCAAACCATGCATAAATACA
TACACACTTGCACCATTTTGCTGGAAATCCCACGTGGATCAATGATATAGTCAAAATAGTGTATATAAAT
AGAGAACAATTTTTCACTTACGAGCTGATTTTGTGAAGTTAAAGTCTAAAGGCAAATTCTAAGACATTTC
ATGTTCCGTATGTCAAACATTGCGCAGGGATGTCAGCGCATTCCTTACCGCGGACATAGATGTCATAGTG
TTGTGCACATCGATATTATCGCTATCCGAGGTTGTCGGGTCAATGCCACTCACCTCCCTGAAGCGACCAA
CGCTCTTTGTTGATGTTCTTTCTGTCAAAGAGCACCCAAGAGAGCTTCTACTGCGAGAGTTGCCAGAGGA
TTCGGACATACTCTGCACGCACCCAATGTTTGGTCCTCAGACTGCCAAGAATGGATGGACAGATCACACT
TTCATGTATGACAAAGTTCGGATAAGAGACCAAGCTACCTGCTCTAATTTCATCCAAATTTTTGCTACTG
AGGTAGGTTAATATCCTTTGTCAATACCCACCAATCACGAAAGAAGAAAGAATCATTTTTTTTTTTTTTT
TTGCATTGGACCAGTTTAATTATGTTAATCAAGAAGAAAGAAACAGAGAGGGTGGAAGCTAAGTAACTTC
TGACGTTTGCATTTGATAAATCAAGATACAAGATAAATCTATGTTGTAAAAAATATAAGTCTCAGTCCCA
CATCTAGCAGAGGTAAGGATTACCATCACCTTACCCTTATAAGTTATTTGTTGGTTTGAGTTAGGCCTAA
ATTTAAACTTACAAAGTATCAAAGGTAGCTTATCTTGGATCTATTAATAGGTCACTTATCATATTACCTA
CACACAAAACCCAATAATGTGGACCGTGAGAGGGTGTATTGAGAAATATCAACACGTTGATTTTGAGAGG
TTAAGCTAGGCCAAGCCCAAATTGAAAGAATCTAAGCATACAATCCTACAGCCAAGGTTATAGAACCTCA
ACGTCATTATTGAAAACAAATGCATATTAAACAATTCAATCTTCAATTCTCCCCTGGACTGAGTTTGAAC
TAATTCTTCATAAGTTTGGACCATTGTTGCTTGGTTTACAAAAGAATCACAAAAATCAGTTGTGAATGCC
TTACATTTATGATTCATCCGGGGTACATCTATTTCACTGTGTTTTGTTACTTTTGTATTACGCAGGGTTG
CAAGATGGTACAGATGTCCTGTGAGGAACATGACAGAGCAGCTGCTAAGAGCCAATTTATCACACACACA
ATTGGCAGGTATGCAGCTTCCTACATATCTAATAAACCATTGAGAAGCACTAATATAAATGCTCACTAGA
TGTAACTTTTGGCTGCTTCTTGATCAGGACACTGGGAGAAATGGATATTCAATCCACACCTATTGACACT
AAGGGCTTCGAGACACTTGTTAAATTGGTAAAGAGTTGTTAACATTTCCCCTACTTTCTCTAAAAATTTT
CCTTTATATGTGGTTATGTATCATATGGAAAAGTTACTTGCAGAAGGAGACGATGATGAGAAATAGTTTT
GATTTGTATAGTGGATTATTCGTGTATAACAGATTCGCCAGACAAGAGGTAATGGAACTGCCAACGAAAT
GCTTATTTACTTTTAAATTCCTTTTTAAAGCACTGAACCAAGTACCCCAAGATAGACTCATGAATTGTGA
AAAATATGCAGCTGGAAAACCTTGAACATGCCTTGCACAAAGTCAAAGAAACGCTGATGATACAAAGGAC
GAATGGGGAGCAGGGTCATAAAAGAACTGAAAGTTGATGCATATTTATTTTACAAGATATTTTCTCTAAC
TCTCAAATATCCTCCTGCAGTTCCAATTATAAATTACTCTTATTTCAGTTTCCTTTTACCAAAATTGAAG
TTCAATTAATAAACCAAAGAGACTGGTATATGTTCAATCACATGCGATAAAAATGTTCCACGTTCTTGTT
CCGAGCAGATTCTTTGTAATTTCATAAAGTTAGAGAAAAGAAAAAAAAAACAGACATTTAGTCGCCAATG
CCTAAAACCATATAATAACTCCACAGTTTGGTTCTCTGAATGAATTCCCTTCATTTTAATCCAAATCTCA
ACTACCTCTTCAATTCTAAACAAATAAATTAGAACACTACCAAGTGATCCTCTGGGGTCTTGACATGAGC
CTTCTAGTTCTAGCTTTTCAATCAATGTATCTACAGAGCATGTATTCCATTTTGGATTATAGAGAAATAA
TGTAAAACTTTTAACCAAATGTTACCGCAAATCTAAAGAAAGTTCATTGCTCCATGATAAATTGATAATA
CTACATAAGATGTACAACTGCTGATTTTATATATCATTTTAACAAGACTTGCCAAGAGATATATCCCTTA
AAGCCAAGAGCACTTATGTTTCGATTTGAGACACCTCTATTTATTCCACTTACATTTGAAAAATAAAAAT
ATTACATTCACCAAACTGGGAAATGGGAAATATCAAAACGTATAGAAGTGAGCCGTGGAAGGAATTGTAA
ACAAATTATGACGAAAACCAGAACCTATTCCTTTGTGCCTATTTTACCAGCTTTTCAAGAATAGTACAAA
ATTACCAAGAAAAAAAAAATGATGCAACGTATTTTCACCGCATTTATTTTTCTCTCTCATCAATGCTGGC
TTCTTCTGTTTCGTTGCATAAAATTGCAGAAGGATTCGGAGGATCTGATGCTGATGGCTCTGTCTTTCTA
ACTTGTAGGCCAGTAAAGCGCGCTAGGTCAATCCATTGCTGCAGCAACTCACGAAAACAAAAAAATTTAA
AACTCCTAGTCAAATTTAGAAATGTATGTGCAGATGAATAATTCACATAAAACTAAATTATACCCCATTG
GTTTTAAAACAATTGCTACTCTTGATTCTTGAGGTTTTAGTCTGTTTCGAATTATGTCACTTTAGAACAT
GAAGACGACATTGCTTTTTTCTTTGTCTTTTATTGTTTCTCTAACTAAAAATGAAAGCAATAATGGTGGA
AGAGGATAATATAGTCAAACGAACTTAGGTTTCCTTGAAATTAAGAATATTTAATGACTTTATTAATCAT
AGTAGAAAACATTAAACAATTATTGTGAAATGGAAGGGAAGTATATTGCAATATGGCAAAAGCAAATGCT
AAAACAGTGTCTTTACAATATTGGCTAAAAAGTCAAAAGAAAAAAGTTATTAGAAAATGTCACTAATACA
TTTCCATTGTGATTTTCAATACATCCCTAATGTAATTTCTAATAAAAAAAAAATTCTATTATTAATTCCT
CAGGACCTTATTACAAATGCTAATGAGTTTCCTTAAGACTCTTGTCAAGGGATTAAAAAAATAAAATATA
TTAAATGAAAAGTAATGTATTACATGTTGTACTTTTCATAAAATGCTCTATATTTTTGGCTTAATTATAC
TTTTGATCTTCTTACTTTTTCAATTTTGTAAACTTTATCCCTCTATTTTTTTCCCACAATTTTGATCTCT
AATTATTTTAATTATCCATTAACTTAACATCATCCAATATTCGATAAATGTGCTGACATGGCAGTGTAGA
AGAGTGTCATGTCAACATGAACGTGTTGACATGCTAGCAACACGTTAACAAGACCCTTTTTTTATTCCTT
AACAAGTACCGTAATGGCACTCGTTAGCAACACCTTTGTAATATTAAGATCACACGTTATAAGACAAGCG
GAAGTTATTTTCTTTCCTTAACTTGTGATTTATTGTTCCTAGACAAAAGCACAAGCTGACGCAATTATAA
CTCCTCAAAACACATATTTCCATATTAACACTTGAATGTGAAATTCACCACTTTAAAAAGAAGGAAAAAT
TAAATTAGATTTTTGAGAAGAATTATAGTGTTCAACCATAAATAAATGAAATCCACTTACTGACGAGATT
GTGAACATTGAGCATTGAAAGTGAACAAAAGCAGATAGAAGATAAGGAAAAAAAAACTGACCTGGGTTCC
CCAATATGAATTGACTGTCCGTGCTACAAAAGAAAGCATATTTACAAAGAATGTTTGAGAAGCAAACTGT
TCAGAAATCCGATGCTCCACTATCCCAGCAATTATCTGCTCAACAAAAATATTATAATTTCCGTATTAAT
ACAAAATAACAGTTCCAGGCATTAAATGGATGTATATTTGTTGAATATGATATAACCAAAGATGTAGTCA
CAGAACAATCAAAAATCAATTTTAAGAAAGAAAGAGCCTTAAAAAGTTATTCAGCAAAGTGCAGATGAAG
AAGTAGAAAGGAGGATAAAATTACAAAATCAGATATAATAGATATTATTGAACCGAGTAATTTTTTTTCC
ACGATTACTTTTAATAGCATCCAGGGTTTACTAACATAATATTTGGTTGGAAAATAATGGAGAGGAAAGG
AGGGCAAAGATTTGGAAGGAAAGGGAGAATGGAGGGGAGTAAAACACCTCCTCTATCAATTTTGGCTCCC
TTCCAAAATTGGGAGAATTTGGAGAGGAGAAAGTTTTACATGAATTGGACTAAACTATCCTTAATGGTTT
TATCCTATTATGAGGATATAATAAATAATAAATTATTTTATTTTCTCTCTTATTTCCTTGTGAACCAAAC
CAAGTGTTCCTCCCCTCTACTCCCTTTCTTTGAACAAAATAGATAGTATACGATGTAGTCCAATCTTTCC
TATTATGCTTGCTACCCACCATCTACTTCAAACACTGCCTAAGTGGCCCGAAAAACATAGTGTACAAATT
AGTTATTCAAGACACAGCTGGTCAAGTCCTGCTTTCAAAATGTCTACATGAATTTCCAAATTGTATTTCT
GTCCATCTTCAATACTATATATTGGAAAAAAATCAAGGCATCACATAATCAACTATTGAGTATTGAAACA
TAAACCATCGACGTGACAATATAGAAGCCTTCATTTCCTACAGAAACTAGGTTCAATCTTCCATGAAGTC
TATTGCTACATATTAGTTAAAATGAAAACTTTATGCAACAAACAAGTTCTCTATATACCTGATAACGGAG
ATTTGCAGATATTCCAAGAAAGCATGCATATATAAATGCAGTTTTGAGTATTGGGGACCTCATAATCCGT
TGTTCAGTGACAACTGCTGGATTAAGGACTTTACGAATTGCATATAGAGAATTTGATGAAGCCACAGCTC
CTATGCTTGAAATAAATCCAACACTAGCAAGTTTTAAACCACCAAATACAACTGATGCAATCCTATGATT
GAGATTCCAGTTTATCCCTGCTGGATTCTTTTGAAATGCATTGTCCGGGATGGAGCCAAGAAGTCCCATT
AGAGAACCAATGTTGTCGGGTGCTTTCATCTCATCAGCATATGAAAGGAATGACAAAGTTGGTGCAGGAA
GCCACACTGTAAAGAAATCAACAACTGATCCTCTGACAGTGTCCGTAATAACATAGTCAATCTCTTGGAA
AAAATTTTCTTTCCGCTTTTCATACTGTGCCAATAATGTAGTTGTTATTGATATAGCTTCTTCTATGGCT
AATCTGTGCAAGAATTTGGGATCTGCCAACAATCTTTCCCTGAATCCCTGTATAAAAAGAACCAGATAGT
GAAGGGAACAAAGTGATGAATGATTAAAAGCAGAATGAAGGTTTCATTTTCAAATAAAAAATTACCTGGA
AACGGTGAGTGAGCTCTGAAATTAGAGGATACTGCTCTAGATCAAAGAAGTTCTGCAATACCTCTGGTGA
TACTAAACCAAGATCAATTCCCTTTTGAAGATCCTGAAATTGAAAGGTAAATATTTCCTATGTTCACCAC
CAATATTGGCACACACTATCCTGTCTAAGGTATGCACATAAAAGAGTAAACAGATGCATAGAAAAAAGAA
CTAGAAAGTAGAAACACACTAACTACCTAATCATCAAATGCAAGTATGCAACTAATGTATGCCAAATTAT
AGACCAGAGGTACTTTATTTTAAGAGAAAGAAAACAGAAATAACGACCTCATTATCACAATTCACCCATT
GGAAAAGTTTTTATGTCCTTAAATTATACATTGCTGTGCAATGAATCTTTCCTCAAAAGGAATATGAATT
TAAAGGAAAAGAAAGATAGCACAAAGACAGCACTACAAAGTTGCAAGCATTCAATTAAAATCCCCCACAC
CAGTAGGTTGAGCTGCATGATTTGTGTCAATTAATAAAATGCAAAACAGAGATATCAATTAAAGGGATAA
GGACCCATTTATTTAAGCTTTTAAAAAAATATTTTTTTTACATATTTTATGTAAAGTTATTTTATTTGGT
TACAATAATTAAAAAATGTACTTTATATTATAAAAAGTAGTTATAATTTTGACTTTTTTTCAGCTGCTAC
TCAAAGTAGCTTCTGAAAATAATCATATAGATAGATAGATTCTGATTTTTTTTCTAAAAAAAAACTTAAA
CAAACACACTAAGAAATTTTAGAAATGATTTTTCATGAAAAAAGTTGAAACAAATGGGCTCTAAAATGCT
CCTGAAATGCCAAAGTTAATTGCATACAAAAAAAATAATCAATAGGTACTGGCACAAGACACCTAGTAAT
ATGCGAAATCTCTTATGTTTGTATCACCAAAATGGACAATGAGAGGACATAACAACAACAACACCACCAA
AACCTTATCCCACTAGGAATGAGAGGACATAAAGGGCTAAAAATTGGAAGGAGGGTCTATGGGGCAAGAA
GATTAACAGTCAAACAAATTAGTAACTGTAATTGTTGGTTTACCTGTGGGAGGGCATCTCGCCTCCGCCC
AGCAGCATTCATAACCCGAGCAATCTCAGCACGGTCAAAGCAATTTCTACTACAGGGTCTCGCAGCAGAA
TACCACAAAAAATCAGCAACAGGAACTTCTCCTTCTCTGCGAATGAATTGTCTTTCAGGGTCAAGTAATA
TAACTGCATGGTTTTTCTTTTGTATTTTTCCTGAAATTCTTGCTGGCACTCCAGTTCCTCTAGATCCATA
TGTAACATGGCTTGCACCAGTGACAACTATTAACATACCAGTGACCCCTCCATCAAGCACATTTTGTAAG
ATAATCTGGGACATAGAATACTCATCAACTACTCGAGCCTGTGCAGAAAGGTATGAGCTTGGACCAAAAG
GAATAGACAGATTTTGAGTACTATCAACAGAAGATCTGCGTGAGATAGAAGTAAAGCCAGATATGAAGCC
TGAACCAGCTGGAGGTGCATATAGTTTACGTTCATCCTTTGTAAGCCCACGAATTCCTTCTGCTTGGACA
GTTCTTAAGATCTGTTGATGAGAAAAGTTCAAGTCTTAATTGTCATCTTTATGTGTCTTGACCTAACAAT
ACAAGAACTGAGCCTAATCCTACAAGGTGGAGTTGGCTAGATGGATCAAATGAAACCTTTTAGCTCTATC
AAAAACCAAAATTGCAATAAAGTTTCTCAGGTTGCCCTCTACCCTTTCAACAGATTATATTCCTTTCTCA
TATCTTGACATGATCAAACTACTTGAGGCAACTTTTCATAATCATATCCTCAATTTTTTTATGTTTAACT
TTCAGTTTAATATGGGAAGGAATCTTTGAGGATTATGTGTTTATTTCCAATTCAGGGTCTGTTCTTTGTA
GACTAGATTTTTCTAGTCCTAACGCAACCAAAATCCTTAAGGGCAACCAAAATCCTTAAGGGCAACTTTT
TACAGCACATAGCTTTTGCACTTATTGGAAGTCAATTTGACAAGCACCAACAAGTATATACTATACTACT
CCCTCCATTCCAAAATAATTGTTGTCCTAAATTGTTTTACACAGACCAAGAAAAAACAATAGATAGATGA
AAGAGAGTTGTAGTTTTACAAAGTTAATCTTATATCATCATTGATTCATTTATAGATTTTGTTTCCATCA
TTAATATTATAAGGAATATACGTGAAAAAATGTAATTAAATATTATATTCAAAACTAAAATAACAATTAT
TTTGGAATAATTTTTTTTTTCTTATACGACAATTATAATGGGACAGAGGGAGTAACATTTTTCTGTTGCT
CCTAATTATAGCCACACCACAACCATAATTTTCAGAGACAAAATAAACATTTGAAAGATCAACATGAGTT
TGGATGAAATTTATGCATACCTTCAGTGGTGTACCACAAGCAACAAGATGAATTCCATTTTCGCGACAGT
AGCTCAGAATAGGTTCATACTCCTGCCATCTTTGAGGCGGCCAATGCAACGTGTAAGACTTCAAGGTGTC
TCCATCTATCCTGCCATGAAAGTCAAGTTTCAGGACAAGTAATGCAGAATTATGGAAAAGCAATCTGACT
AAGACAAAAGAGCTTCAGAGATTAACAGAAAATAGTGAGCCAGAAAAAAGATTGCGAGACAGAAATTGGT
CGCCAACAAAAAGTTGTCTCTTTTATAATTTTTAATTGAAATTTTCTTAATTTAGCTAACATGACTTCCT
ACGGCCACAATTGCGTTTGCAGACACTTAAAAAACTTGATGTTGCAGCAAAAATCACGTTTTATTTATTA
TTGATGTCAATTATTTAACAGTTTTATGTTAGGTTTAATAACAGTAGGTTGATGCAAGAGGCTAAACATT
AATCAGAAATTGAAAGGCAGTGTTATTACTTCTTATCCATATACTGATTGAGCGGTTCCTGAAGATTAGC
GGGAAAAACTTCAAGCGCCAGAGACAATAGTTTTTCCTTCTCCAAACAGCGCCTATGCAAATTCTTCACA
ATCTCAAGCTCCAATTCCCTATCGTCTCGAACCGGAACTTGCTCTGCTTCACCTAAATACACCACTCGAG
CATTCATCAACTTCTCCCACACTTTCCCTTTCTCTTTCCCTATCGCCAACGGTTCTCCTATCACCGTCGC
GTCGTAAATCCTCGAAGTTATCACTTCCTCCTCCTCCTTCTTCTTCGGCGGCTCCTCCGGCTTCGGCGCC
GAAGCCAGAGGAGATTCCGCTGCCTTCTCCTCCGCCCTCGCCGTCGCCGCCGAGAGCAGGATTGATGCGC
CGGCGACTAAGAACGGCGCCATCAGCACGCCGCGCCGGCTGCTCCGAGCTCGAGTATCGCCGTCGCCGTC
GGAACCACCGGGATTCGAGGCGGCGGTGACGCGACTCGCGTGGCAGACGGAAAGGCTGACGCGGCGGCGT
TTGGCGGTCGAGACCCGGCGGAATTCGAGGCCTCCGGGGGCGTCATGAGGAGGGTCCGGTGGCGCGGCAC
GTGCGGCAGTGGCGCCGCGGAAGTAAGGCACGTGCGGGAGACGAGTGACGAAAGAAGAAGCCGGAGTGTG
GGGCTTCATTAGTTTCGTTGGCTTCAGTCTGGCCTTATCATCAACCGCAACAAGGTTTAAGTTTTGTCAG
TTCACTTTTTTCAACTGCCACACAAAACACGAACAAGGATTCCTTTATTTTCAGCATTACAAGATTCCAT
AATTATTATTATTATTATTATTTTGGAGAAACTTTTTTTTTTCCTTTCAAATATCCCTTTTCATAGACCA
TTTTAATTGACAAACAATTAAACATTAAATAAAAAAACATTTATTATCAAACAGTAGATCAATTTAGGTG
TGTTAGTCTAAAGAAAAACAAATAAATAACAATGACATGTTTTGAGAGATAGACAAATATTCCCTCTAAC
ATAACATAAAACACACACACACACATATATATATATATATATATATATATATATATATATATATATATAT
ATATATATATATATATATTTGTGGTAGAAAGAAATATTAGTTTTAGTTCGACATATTATAAGATATTAGT
TTCTTTATGTGAAATTCATGTTCTTTTATTATGTGTTTAATTTAAAAGATAAAATTATATAATCTAAAAA
TAAAAAAGAAATTTTTGAATTAAAATAAAATATAAGAGGTGTAGGATTCATATTATTTTTTCCTCAACCA
AAAAGACACCCTAAAAAAGTTACCAATATACTTTTTAACATGTTATTTTAAAAGTATTTTTATTAACTTA
AATTTATTAAAAATTATATTTTTTTTTAATTTCATCTTTTATTCAATGAATTTTCCCTATAATTTGATTG
TTTTCAATAAATTATAATCATTATTAAAAAATATATAATAAAGAAAGTGTATCAAGAAAATATATCAAGA
GGTTATTCTTATTTTTGCCTCTCATAAAATCCTAGACTTAAAATATACACTCTACTTTTTGTGTTCAATA
ATAAATTCGTATATTTCGTGGTCGCAAAAAAATTAAATGATATTGATAATTTACATAGTCACGCAAACTA
TATATAGAAGGCCTCAAATTTAGAGCTCTACGTGAAGCATTGGTGTCCATAGAGTTACATGGTGCCAATT
ACCATATCTTTCATTATTTTGGAATTTTCATGTAAGATAAAATCGATCCGAGTCAGATGTGACTCAGGTC
TGAAACACAGGTGCCGATCCAATATCCATGTATGCCAATAATACGAGGCGTCTACTCTAATTTGATTGAA
AATAAGGGGGCAAAAAGTAAAATATATACTGCCAAATTCCAATTCAATTCAACTATACGACATTGTCTAA
AAGTTAGACCAAATTGACCAACTGAAGTGATACCTCTTTGTTGATATAAAAAACTCGTGTGTGCTATGAA
TATTTTTAAAACAAAATAATATTTATGAGATAAAAATATCATTATTGTCAAATAATGATGAAATACTATC
ATGTAATTTAAGAAGAAAAAATATATAATAGAAGATGACTTTATTGTTAATTATGTGAATTCTATTTTAT
TATATAAAATAAATTAAGGTTATGTTTAAAAAAATTAGTTGAAAGTTAAAAAACAAACTAATTGATAACC
AAAAACTTTTAAGTTAATTTATTAAATTATAAATATTTGATAGAATTGTTGTTGAAGTAGATAAAAAATA
TAATATCACAAAAATAGATATATTTATATGATATTTATATAAACTTTAATGTTTTATGGACAAAAGTATA
TTGAGGTATTATAATTTTATTTTTTAATTAATTTTAAACTCTTGTAAATTATTTTTCATTATATCTTTTG
TTTCAATTATTAGATTTTTTCATGTTACATATTCTATTATTAATCTTGTACAATGTCTTAATATTTTTAA
CCAAGTTTGAAATAAAGTTGAAAAAACATGCAGTAAATATTATACTTTTATTATATTAATTAGTATAATA
GTTAAAATAAATTGTATAATATAAAAATATTCAGAAAATAATAAATTATTTTAACATTTTTTACTGTCAA
TTTCGTGAAGATGTTGAAATAATTACAATGGCTAAGACAAATAGTATAATATAAAAAAATTGTAAGAGGA
ATGAGTGAAAAATAAATAAATAATAAAATTATGTTATATTTAAAAGGAATAATAAGAATATTTAATAAAT
ATTTTAAGAATTAAAAAATAAAATATAAAAGCAAAAAATTAGAGGCTAAAAACTAGAGTTTTAAAAAAGT
TACTTTAAATAATGTTTCACAAAATAATAAAAGTTACAAAAAAATACTTATTTATTAAATAATTAAACAA
GTTTTTCAACTAATAAAAAAATAAAACTAACTTAAATAACGTATCAAACATAGCCTAAAGCGAATTTTAA
AAAAAAAATTAAGGTGGAAAAGCATCAAAATTCAAAGTTGGTATCAAAATTAGGATTAACTAAATTTAAG
CAATAATATATGTATCCTTTTCCTCTCGGCCCCAAAGTTCATGATCAATCTTATCAAACCTTTTTAACTT
ATCAATTTTGCTTTCATGCAAAATCTAGTAAGAGTAACTTCAAATTAAATCCACGTTTGATAGTGAGACT
CAAGTTTAAAATCATATCTATCTTGCTATATGTAATAATCATGTTTGACTGCTGAGTTTGATGGGTCAAG
ACTTTCCTAATAAAATAAAATGTGGGTACGGATGCTTAGTTTTGATGGGTGCAAATACAATTGGAAAAGG
TATGCATCATTAACAATGTTTTACACGTCTAATTTCTCCCCCTCTGATTCTCAAGAGGCAGGGACAAACA
GATTTCACATGCCCTTTTCTGGGATACAAACATGGTTTCTCACTTTCTCATGGTTTAGCCTTAAAACATG
ATTCATCGCAATCTGCCCTTACCATTTGGGAATGTGACTGAATAGTTTGGTAACTCAGAATTTGCTACAA
TCTGGTGATAAGTAATGACTATTAGATCATAATTTTGTTGAGAATACAATATTTCTATATATTCTAATAC
TACAGTGTTTCTATTTCTGGTTTCAAATCTCAAAAACAAGTTATACACAAATTCTTTTGGGAGAAAATAA
ATAATAATAAAAAGGCAAGCTAGCAATCAAACTCCGCAACTAAAGATACATAACGAGGTGGTCACAGAAT
AGCTTATCCAGTACAATTTAAGAAATTGGTATACAAAGTATGATTTTCAACAACGAACCTCCTTTACCCA
ATATTTAGTCACATTTATTTGTAACCTATTAAAAACTTTTGCGAATAGCTCCCCTAATAAAAAATGCCGC
ATGATTAATCATCAACAGGAAAAGGCTAGCTCACTTGATATCATGAAAAGAAGGCAAGACAGCAATAAGA
CGGTCTCCATAACCAACAGGCTCTGTAAATAACAAACAAAAAATAGTAAGGAATTCATCACAACTATGGA
TGACTGATTGAGTGTAGCTGCCATAATTGATGGCCTAAAATATGTTTAAACATTGATAATTTGGTTGAGC
ATTGACGTTGAACTTCAAATATTGCAAAAGGACGGAAATCGCAATGAATAAATCACTGAAAAAGCATAGC
AGAAATTAAGACCTTAAGTAAACAATATTTTTCCATTCAGTCAATAGTCATATACTGACTAGAAAACCCA
TGAAAACCGATATACTGTAAATTACAATGAGCTAAATTAATTTATCATGAATATCCTGTTACTTTCCATC
ATTTAGCAAAAAGGTATACAAGATTCAAGATCCCCAGTGTTTGGTATGATTACAAAAAAGTCACATTATT
TTCCACTTTGTTTTCTGTTTTAAGATATTTTTGTAGTTATAACTTATAAGAGAAAACAAGAAATGTTTTC
TCAAACTCTATTAAGCCTAGATACTTATGCGACCCAAATACGGGGGATACGGGAAATTCTTAAAATTCAA
GATACAACGCAACTCAGATGCATTAACACAAATATACACACACACACAAATAAATAGAGAGAGACATACA
TACATACTTTAAATAAATGCACAGTATTTATTAAGAGACATTGATTATCTTACACTAATACATAACTATA
TCAGTGGACGATGATCATAATTCACAAAAGCAATACCTATGATAATAACAAAATAAAAAAACAAAAAAAC
AGTGACATATGTTTCCATTACTCATACCAGAATATATTATTTTCCAACATGCTACAAAATCTTCCCCCTA
ACTTGTGTCAAAATGTCACAAACATCATCATTTGTCCTACTCAAAAGAAGAATTGACTCTATATGATTGC
CTAGTAGTAATATTTAGTATTTACTATAGCTTTAAAGATAAAGCTGTATTGAATTTTATTCCTATTCAAA
GTACTGGAGCCATTCTCAACCATACCAATCTACAAAGTTTTGGAAAAAAAAATAGAGGATACTCTTTGGA
ATTGGATAAGTACAAGAGTATCATATGCGTATCGGTGTTGCATACAAGTACAGCATAGATACTTTGTCAT
TTTTGGAGTATCAAGGCTTCACAGCTCAAACTAAACAAATCAAACCCAACATCCCCACGTTTTAATATGA
TAACAGCATGCTAGCCATAAAGCAATTAGGCATCCATGATACTAGAGTATCATAAATACAGGTCAGAAAA
TCAGTACTAAGTGTTCCAAGATGGTAAAATTCATTTTTAATGCTTATCACTGTGACTACGAGAATTTAGA
ATTATTAGGATTGCCAAATCATTTTTGCCATATTTTATTGCTAGAGGCACACTATTGCTTTAACTATTTC
AATTTTGGATGAACAGCACGGCTATCATCACTCTTTCTTTCCCCAGAAGCCCTGTATTACTTTAGTACCA
TGTAAATAAATCTATACATTTTGGTAACAGGTCATAGAAATTATTATACCTCCATCCTCAACAAGTAGCT
TCAACACTTCTCCAGCCACATCAGACTGCAAGATTTCAAAAATTAAACTAGTCAGAAGTAGTAAATATTT
AGGAAGGAACCAGAATTACAGAAACAGAGGCATCACCCTGATAGGAAGTCCAGTGCCAAACTGATCCAAA
TACCCTATGACTTGCCCTTCTTTGATTACATCACCCTAGAATTCAATTAGGAAATAAATATATTGAAAAG
AATTTGTAGTCAGTTCAATGAAAGTGAGGTCCTCAAACAACTTGATGCAGCAACTGTATGATACAAAATA
TATTAATAACTACACCAGCAGAAAAATATAGGTCAATCTATATTTGGGAACCAAATAATATTTAATTTGT
ATCTGATAGACTCAAGAAATTATAACTAATTTGGAAGAAATGGATACCTAGTATTATTAAAACACCAAAA
CACAGGGCAGATTATAGTAGCTAAAGAGGAAGAAGCTAACTAGTCAAAGTGTCACACTATTCAACACTAC
AAAGGACCAATCCCCTTTTAGAGAGCCTGACCTTTCTCACCCAAGAGCTACCCAAGAGAATACACACCCT
CTCCTCCATATCCCCTCCCATATAACACAATCCTCACCAACTAAGCACCTACCTGACAATTCCCTCCTAA
CCAACTCTCTGCTCATCAGGGTTGATTCTCTTCTCTTTCCAAGACTTTGGGCTTTTGTTTTGACTAAGCC
AAATTTCTATCTGCTGGCCTGGTCCAACAGTATCTTTTACAGACAAGTTTACAAAATATTCGTATTTGTT
AGAATTTATTGATATTCCTATTATGTCCCCACTGTGTGCAAACATTTAGAAACTAATATTACAATTAACA
GTTTTTGTGAATGCAGCAAAACTAAATATATATGATATAGAAATCAACAAAACTGAAAAATTATATGCAA
AGTTCAATTGAAAAGAAAATTGATTACCCTTTTTGTGGTAATAAATATAATGATAAACTAGGTAGGTTAC
AGTTTGGATTTGTGATCAATTGAAGATCTAGATGCTAATTGGTCATAACTACAATATTTTTTGCAGTGAT
TCTGTGCCTCACATCAGTCACGTGTGTCTATATAACTTGTTCTTAAAGTAAATATTAAAATAATATAAAA
AATATTAGAAATTTAAATTATATTTACATTTTTTAAATGTATTAGTAAGTTTTGTTTATATCAATAATCA
ATTTTTTAAGATAAAAATTTACAAAAAAGTAATACAGTAATAGAAAAATAATTATCAATATTGTATAAGC
TGAGACTATCATTGCTAATTATTATCAGCTTTTCTTTTTTATAGCATCCAGTTTTTTCAGTTAAATACTT
AAATTTATTTCAAAAGCCAACATATGCATGTCAGCAAGGTCACATCAGGAGACTAGGCCGACAAGCAAGC
AGGCTGATACATAGACTGCAACTATTAGTTTCAGCACTGCAAAATGTTAGTGAACAACAAACACATGCAC
CAAATCCAATGGTGGAGTTCATAATGAGAATCCAGATGGAGGGAGTATCAAGTGAATTATGTGATCTAGT
TCATAACAAATTGATGACATAAAAACACCAGAGAAGAGGATGGATCTTGACAAGCTTATCACATGTACCT
CTTTACAGATAGGAGGTTGCTTCTTCCCTTTCACTGTTCTACCTCTTCGGAATAAGCCAACCTAATGAGA
AAGAAAGATCTGTGATAGCTAACTCTACATAGCTCAAGTCAGAGATAATTAGTGAAAGCAGAAGTTAAAA
CATATATTATTGATGGATGCCATACCGTGGGAGATGTTACTAAGACATAGGTATTGGTCCCAGAAGCCTC
CAATGCTGCCAATTTTGGTGATTTCTCTTTGGAAACATTTGCAAATGGGTTGTTCTTTTCAGGAGATGAT
TTTGGTGGTGATGGTGGCAGGCTATTGGGTGCTGATTCATCCATAGGTTTACTTGGGATAGGTGGTGGTG
TAGTTGGTGAAATGTTAGACAAAGGAACCTTTGTTGCTCCAATGTTTCGCTTAATATGCATTTCAAAATC
TCCAACCTATAACAATATAAGCCAAATAAGGTTGTGTACAAGGTCATCCAGCTAAAAGCATGCTTCTTTT
GATGAACCAGACAGAAATTACAGATTTGAAGTTATATTAAAGTTATTAGGCACAATGTTGTATGAAAACA
AAGCTGAGCTGACACAATTTTTTATGATCAAATTTGCATTACAAGAAAAACTATATATTCAGATAGGACA
TTAGGCACAATAGCACCATATTCTCAAGTTTCAATTACAATGTGCTACCATTTTGATAAAAACATAAATA
CCATTACCTCATTTAATAATCTATAAGCCTGTGTTATCTTAAGTGAATTAAAATCATCCTTTGACATAAG
TTCAATACACAGTACATGGCAGGCTATAGACATATCCAGACACTAATTACGAATTGCAAAGAACCCTACC
ATATAACATTTTGCTTTAGAACATACACTTAAAGTTTTTTTCATCCTTGTTATTATTATATTTAAAATAC
TGCACTTCAAATTTTACAGAAGCAGTAAACAAGAGAGAAGAAGATCACCTTTACTTTCAGTTCAGCAATT
TCAGTCTCATCACAGACCTCTAATACCAAAGCCTGATAATAAGCAAACCAGACATAAGTTGTGGTTTATC
AGATTCACATTGAAACATGGCTGTTAACGAATGCAATTATGCAGTCTTGGATAAAAACCAGCTATCATAA
GTAAAAAATACCTTGTATTTATTTCTTCTAATAACTAACATAAGGTATTTGTATTCATGAGCATACAATT
GCCAATTCCCACCAAACTATCATTTTTCACTAATAAGATAAGATTTTCAGGAAAATAAAAGGAAAGAATA
GCTATAGACCTCAAATCCATTAGGAAAAGTAGCAGTTTGCAAAGGCTTTTTCTCCAATGAGCCTTGGGGG
GTGTTATCTGAAGAAGCTGCAACATCATAAATAATTGTAAGGTCATGGTTTACGAATGTTAAAGTAAAAG
TTGTTATAGGGAAGCTATTCTGATAGCCAACTATAACAAAGCTGTATATATCATATAACCTTCAAACAGA
CATTCACAATTACCATAATAATAATTAGCAACAGAGTAGAATGATTTCCTACTATTGTGTATCTATGTAA
ATAACATAATACCTTAAAAATAAACAGATAAACAATAAAAACAACATCTTTTGTTTGTAAGGATTTTCTT
CTATCTTCTTTCTTTCCAACAAGTATATAACGGTTCGGCAACATTATCTTTGATCATGTATATAAAAATA
TGAAATTGCAGCCCAAAAATTTTGCAACTGGTTAATGTATCTCAAAATCTTAAGAATAAAAACAACTTAA
AGTTTATTACTAAGAAGATAATTACACTAAGAAGTAAGAACAAATAAGAGTATCAGACTTTTCTCATTCA
AACAACAGCCAAATAGAACAAAAACATAAGGATATAATTCATTTCACAATCAATATAAACCCACCATCAG
AATTGGATGTGTTGATAGCTTCAGCTGTTTTTGCAGATGAAACTAGTGTGCTTTTCCCCTTCATGTGGGA
ATTAATGTGCTTCTGACCATATGCCAAATGCTGGATGAAAAGCCTACGTTTAGAGTTCCATCTGGCATTA
TGGATGGGAAGTACAGCTTGCTTCTCAAGGCAGGCTCGCACATGGGACATAGTGCCCATGGGATCTGAAA
TCCAAGAGTCATGATTAAGCATCTGGTTGCAATGGTAAAATTTGCCCCTTTAAAAATATAAAAAACATTG
AAGTATTACAAAAATATTGTATTATGATTTATGAAAATTGACCCTTTAAGAAGGCATTAAGAATCTTTCC
AAAAAGAAGTAAGCCTATTTCAAAAAGGCTTGGAAAAAGAACGGAACACATTGTTTTATTTAACAGAAAT
GGATCAAGACAAGAAAAAAAAAAAAAACCAACATAACACAAATATTTCTGACAAGTGTTTCCAATCAAAA
ATAGTATACAGCCTATGGGTACCACCAAAGTTAATAAATAATAATTTTAAAAAAAGAAAAACACAGTCCC
TTGTGTCCTACTATATGACCCAACAGAATGCCAATTGCATGCTCTTAGGTGATTGCAGAGCATCCTTTGG
TTATATATATATACATATATATATATATATATATATATATATATATATATATATATATATATATATATAT
ATATATATTTGTAGAAACATGAAGGATACATTCAACTGCCTTCATGAAAATGTTATGCTCGTGATTCTGA
TAAATACGTGACACTTAGCATTAGCATAAAATAACCTGTCAATGCTACCTCATTTTTCCGTTAATTTCTT
TGGTGTTATTCTTCAACTGTTTTTCAATTTGATTTTTCCTCATATATGTCACATAAATTAAAGCAAATAA
AACCGAAAAGCAAGAGAGCAAGATCAGAAGTCGCAAACACACGAGCAGAGATGGCAGTCGGCAAAGCACG
TTCATAACAAAAAAAAAATGCAGGTAGAGATGAGGAGAGAGAGAGAGTTACAGTGAAAGGAACGAATGGC
AGGCGAGGATTCCATGGGAAGAAATGGAAATGGAAGAATGGGAGGGAAAAACAATGGAGGAGGAGAGGAA
CTTATAGAGAAGAGAGAATAGCCAAGTTGAGTTAAGCGAATGAGGAAAGAGGTCGATAAATTAGTTGCAC
TGTGTCTGCTTTGAGATTTCCGCCCCTCTAATCACCTTCTCCGTTTCAATCTAGGAACATTGCCTCGCTA
ACGTGCGCCGGTGTGTGACTAGTGCTTCCCTCCTCCCTCTTACAGTCTTACGTGGGACCCACCCCTCCAG
GCAGGTAGGTTTCATGGACAGCCATACAATGAATAGTTCAAAAAGTCTAATTTAGTAGTTTCTTGTTACT
ATATTTTTTTTATGCAGTCCACACTAATAAAAAATTAGATGGTTGGAAAACAAATCTTATTACAAGTTTT
ATAGGTAAACTTGAAAAACTCTATGTTATAAGACCTTTTTCTCACTTTGGTAGTAGTCTCTTATTCAAGT
TAGATAATTCTTCTTATCTTAATAATAATATTTTTTTTTATAGTGATAGTCATGGATGTTATTTAGTGGG
ATTTTATTATCCCCTCTCTCCACCTACTCTTTCATTATAGTAATGCATTCTTCAAAGAGTCAAAATACAT
TTCATTACTTCCAAGAATAAACCTTTTAATTTTGGATAGATTTATTTTTTAGTCTTTTAATTTATTTATT
TTTTAGATTTAATTTGGTCCTTCAGTTTTTCAGAATTCAATTTAATTCTCTAATTTTTTAAATCGATCAA
ATTTGGTTTTTCAATCTAAATTATAAGAAACTATATTTTGTGATGGTTTAAAATCGTCATTAAGTGTTCT
TAAGCTACCACAAAAAGCACATTTCCAAAAAAATAAATTAATTTTAAAAATTATAAGATCAAATTGAATC
AATTTTAAAAATTAAAATATTAAATTGAAAAAAAAAATAAAGGATCAAATTGAACATAAATAATAAATTT
GAGGATTAAAAAACTAATTTAACCTTTAATTTTTTCTCACTTATATTAATATTAAAAAATTATATTGATT
TTCCTAATAACTCCTTATCTCAATTAAAATTTCCAAAAATTAATTCTAGCATCTTCAAACACTACTCACC
ATGAAAGTTCATCACAACCATCTTTCTTTCTCTTTTCTCTACATCATGTTTTCGCTTCGCAAACTTTATT
GTGTTCCTAGTCTTAGACGTCTGATAATCTTCCACAAGTATTGAACTATAACACTTATTGTACTTGCACC
GTTAATAGCTAACACCAAATGAGACGTGTCACTTGACTTTTATATCACTAAGAAAATTTCAACACATTGA
TCCAGTATTAGCTCCATCTTGCTTTAACACTTGTTTGACTAGTCACTTAAGTGCAACAACCAACTTTGAT
ATCATTGTTGGAAAAATAAACCTTATTAGAAGTTTCCTAGACAAACACGAGAAACTCTTTCCATTACAAG
ACTTTCTCTATTACTTGGGTATGGTGGTGACTTCCTTTATAATGGTGGTGAATAGCTCCATTTATAAATG
TTATTTAGTGAGTTTTAATTATATCATCTCTCTATCCACATTTTCATTACGCTAGTAGGATTCTCCAAAA
ATCAAGTTACATTCCATTTTACGTCACCTCTTAATTTTTTGCTCAGTTGCTTTAATATTTGGAAATTTGG
ATTGGTTTTCACAACATATACTAGATATAACTTTTAATGTAATTCAAAATAACAATTCTTGATAAATTGA
TTTTCACAACATATTCATATATACTCAAAATTTAAAGATAAGTACTACATTTTATCAGGTGGGTCAACAC
ATTTTACCTCCCCGATCATAGAGTGATCAGGAGGAAAAAAAGAATGAATGAAAGGAGGAGTAAGGTGAAG
GAAAGTAATGAAAAGAATGAGACAACTTTTAAAAACTTAAAATTAAGGATAATAAATTTATTTATTGAAA
TAAGGGTTTATTTTAATCAAATAACCAACTTTTTTGTTTTTTTAGTATGTTTGTCTAAATTATTATTTTT
AAAAAATAACTTTCTGTTTATTTTAAGAAACAAAACAAATCTTATTTGCTTTTTTAAAAAATACTTATAT
AAAAAATATTTATTTTAATTTTTTTTAAGTTTAAACAAACTCATCCTAATATGAACCAGAAAACCTTAGT
TTTTGTTAACAAAAATGAGTTAAAATAATTTTGGATTCATTTTAAAACTATTTCTTTGTCTTTTTAGTCA
TTCAAATGATTTATTGATAAAAAATTATTCAAATATTTTTGTTTTCAAATTTAAGTGAGGAGTGATAAAA
ACACTTTTTTTATTGGATAGAATTTATTAAAATTTACAAAAATCATGAGTGAAGTTAGAATGATACATAC
ATATTTTGTCATTTCCAATAATTTTTAGTGAAAATAAATTGTATTAAAAAGTGTGTTGCTATTTTTAGCA
CTGAGAGTCATGAACATGGATTTGCTCTAGGAGTGATAATTTGTGGAATCAAGTGAGGGAGAAACTCATT
TTTCAATTTAACTTTAAAAACCAAAACTAAAAAACTTAAAACTATACATTGTATTAATTAGCATGTGTTT
TATATATATATTTGAGTATGGAAGGAGTACTCTATTCAATGAGATGAATATGTGTTAACAAAAAGATTGA
TTAGGCGATTAAGAAAGAAGAGAGATTCAATTCCTCTTACCACTAAAATCTAATAAACTGATAATTAACA
TTTGTTAATAATAAAAAAATGAAATGGACATGCAATTAATTAGGCCAATGATAAAGAATATATTTAAAAC
AAATTGTTAAATACAGTGTGTTTGACAATGATATATAATCGTGTCCATGGATCATATCAACCTGAAACTA
ATGAAAGGATCACAGATCACTTCTATCTTCCAATTAAGGAATCACAGGTTTAAACAATAGTAGTAGTTAA
TTTGCACATCACTACTCTGGAGGCAAGGCTAAGCAACGTCGAGATGGACTATTTCTCGAAACTCCAACCT
CCTAGTTTCACTGTAGAATGTCACACATTTTGTTAGACCAATATGTTAGCCATATCCAACCCCTTATCTT
CCATTCCGTTGTTTTCCCTATGGCTCCTTTGTTCACTTTCCACCACACTTTTTTTCCATATTCATCACCT
GCGAATATAACCCATCCTTCCTTGTTGAAAGGTCAGTAAGCACTGGAGTATCGATTTTTGAACCATGACT
TTGGGTTAAGCATTGCAAACTTCGAAGGCTTTGATGATAAAACTTCTTTGCCATCAACGCAGTCATGAAG
AAACCAATTCAAATTTGCACTGTATCCATAAACTAACGGTTGAGGACTCTCTCCAGCTGACACCTTAAGT
GCCAAGGGATTCGATGCCTCAAATGAAGCTTCTACGCTTTTGTCAACACCAATTTCTTTTCGTGGGTTCT
CTGAGGATTTGCCAACTGATAGGCTCAACACTTGGTCTTGGATTGTGGGTGTAAGTTGCAATGATATTCT
GGAAGGAAAATGTTTTTCAGCTGCACCAGCCCCTCGTTGTTTCATAAGGAACTCATTCACCAATTCGATA
ACATCACACCTGTCCACATGGAATATTGTAAATTATATTGGAGATTAAGAATTTACGTTAAAAATTAAGA
GTGAATTTGTTTAAACTTATAAAAATTAATTTAAAAATAGATATTTTTTTATATAAAGGTTTTTTTAAGA
AATAAATAAGATTTTTTTATATATTAAAATAAAAAAAATTATTTGTTTAAGAAAAAATAATTTATAAAAA
TGTTTTAAAAAATAAAAAATTACTTATTTTATTAAAATCAATATTTATTTTAACAAATAACTTTAAATAA
ATTGACCCTAAATTGTACACAATTAATTAAGAAAAACAGTTACTCAACATATATATATATATATATATAT
ATATATATATATATATATATATTACAATCTTATTTCCTTAGGTGAGATGAATTAGATAGATCACATATCA
TCATGACTCGATTAATAATCTCCTTAATACAGTGAAGTAAATATATAAAGATAATGCGATTCATAAAAAG
AGTGATTTTGATCACCTTTGTTATAAAAAGAAAAAGAACGATGCTTTTTTTTTTTTTGTAAATATAAAAT
TTAAGTGACTTTTAGATATTTCCAATCTAAATCCAAAATATTGAGCTATAAGAAATTGAGTTTAAGTTGA
ACAAAAGTATATCATAGAAGGAAGAATTTAGAGAAAAGAAAAGAAAAAAGTACATACCACACCTTGTAAA
CAATTCAAGCTAATGAGCCCAAAATTGTACAAATGATGAATTTTTTTTTCCTAAACTTCTAATAACAACA
TGGGCATAAGCTGAATAATGGATAATTTATTTGTCTTGTTAAGATGCTTATTTATTGGCTCTATATGTAT
TATCTCTTAATGGGATATTAATGGGATAATATTAGTGGTTAATTAATTAGTGGTTACCTTACGTTGTCAA
TGTCCACCATGATTTCAGCCCACTCGTCTTTGATTAAGACTTTGTGATTGAATTGAAGAACGCCTTCCAC
ATGGTGATATCTGAGGGAGAATTGAAGCCTCTCGCTAGCTGGACTGCTTTTTGAATTCTCAACATCCATG
ACCATGAGGTGACAATACAGCTGCACCTTCCACAACCCAATTGTTGAAAATGCATAAGAAAACAAAGGAC
ACGGTGTTCTGAAGGGGTTGTTATGTGCTTCCAAGTTCCCCAACCCAGTTACTGACCGCAAGGTTCAATG
AACGCATCCACAGCTCTTCCAAGTTAGACCCTAAGAGTTTCATAATCATATGTGATGCTTGTCTAGACTG
AAAACCTATCAAGTGATCTTTCAAGTTACTAATGCACCCAGAACGAAAATCTGCAGCAGGAGCTTCATAT
ATGTAAACTAGGAACAAGAGGGTGAAGAAAGAACGTCTGAAAGGTTGAAAACGTCTGAAAGGTTGTCGGA
AGCAGTGGAGTCAAGGTTTGGAAATCTGATGAAGGTAGTGCTGTTTTTGTTGGAGCCATAAAGAAGGATG
GCCTGAATGAAATTAGCAAAGACATTGTAAATGGTTTCTTCATCATCTATTAACTTATTATTGGTGTTTT
TAGTAGTGCTGCTAATTGGCTTGAAAGGTTTTGAGGTCTAAAGATGGATGGGAATGTTGCAGTCTGCAAT
AATGCCAAGATAGAGGTTTGAGGAATGACTATTCTTTTTGTGTACGGTGAGATTAAGACGTGGTTGGCAT
GAGCTTGAAGAAGAACATATGTTTAAGGACATGGAACTTGTTTCCCATTCTGAAATTGGTGGAAGGTTCT
GAATCCAGCAAAACACATCAAGAAAGTTGTTAGCCATGGATCGAATGAAGCAACTTAATTAATATAACTC
TCTCTCTCTATCTCTCTAATTCGGTTGCATTCAGGTGTGGCTTCACATTTATTTGTAGACTCTTACATAA
TGCTATGTTATGTACTGCAATTAGCAAATACTCTTTCTAGTGGAGAAATAATAATTAAAAAAGTGGACTG
ATTGGTACGACCATTAGTTTAATTAGCTCCATGGAGAAAAGCAAGATAAAATTGCTAATTATTGGTTAAG
AAAATAATTGCACCAGATATATTATATAAAATGTCAAAAACGCATTCCGTACATTATAAATAATATTATA
TACGTCATATTTACATCATTTTTTATCCTTGTTTATCTCAAAAAAGTGTAAATATAGAGAGAGTATATAT
CATATCATATAATATGTAAGTTTTTATTAGTTTAAAAAAATAGCTTGAGAGTAATGTGATTTGTCATGTG
CTAATAAAATATCATTTTGAATGCTCTTTTATCCACATATATTAATTGTTAATGATTGAAGTTTATTATT
ATTATTATAATATCCTTTTAACGATGAAAGTTTGTTTTAAAAAAATATAGATTTAAGATGTGTTTGGAGG
AATTTATTTATATCTTATCTGAACTTATTTTATGGCATACGTGTAAGTATTTAAGAAAACTTATAAAATT
ATAGTTTATGATTTATTTATAAATTGTTTTCAACTTATTTTAATAAAATTTTCAAAATAACTTATAAGAA
CAAATTAAATTTTTTATATGAAAATAATTTAACCTTATTTTCTTTTCAATTATAAAAAACAATTTACAAA
TAAAAGCTTATATATATGATACACACTTTTAAGTGTTTAAGTAAGCTATCTAAAAAAGGCCGTACAGTGT
TTCTTTAATGAACTATCGATCGGGAATGTTATATATGGAAATATATATACTTGAGTGAATATAGGCTCGA
TTACTCCATAGTACAGTCCAATAATTATTAGTAAACGAATTATACGTTTAATTTGTATCTATATATCTTT
TGTTGATAATTGATGTAATTTCAATTTTAATTTACCAAAGAGAGTTAGCACCACAGCGAGCATCCGTTGC
CTCATTAGTCATTAGTACTTATCACCGACATCTTTTTGTTTGTAAAAGGACCACTGATTCATTTACCTAC
ATATATAATATACAATATGTATGTATACAAAAATCATAGTAAGGTTTAAATGTAATGCTTCATGAATAAG
ATATTCTGTGTTACAGATTAAGATTCGTGTATGATAAAATGTTTGTTATTATTAGAGTTAACCGGCAATT
TGTTCATATTGAGTCTCATTAATTACCTTCTTTTCACATGTTTTGTTGACATCGAGAGTGACGATCCTAC
CGAGATAGATAAGGATATATATGATAACAAATTGAGATAAAAAGCTCTTTGCACAGTCAATTATGATTAA
GAAAAATATCAAATCAGTTTTACAGACCGTAGCTCATTAGGCAGAGATAATTACATGCACGTAAAGAAAA
AATTATTGAGTCACTAAAATTGGGATAGCGAGGAATTTGAGTAATTTGAACTAAGTCATAAGTTTAAATC
GTATCGTTAAAAAAAATGTAGTTTTTGTTACTCTTTTAAATACTAGTATTTTTTTTTTTGAAAGGTTTTA
AATACCAGTATTATTCCACTAATAACCTGCCTTTATTTCTTTATATAAAGCCTTCTCTTAATGAAAATAG
AATACTAATTAAATAATCGAGAGAAAAAAGATACAAATGGAGAACAAATTATCATGAAAAAGTTACACAT
TAGAAAATATACATGTTTTAGCATTGAAAAATACAATGGTCAATTATAAACCAAAGAGGCCCTTAGTTAG
TTAGTCTAATGTTTAAGCCACCAAATTTTTGGTTGATAACGTTTAAAAGTAATAGCTAGATGGTCTCTTT
CAAAGAAATTTCTGTCCATATTATTCAGGTTTCAAATTTTGTTTGTAAGACGAGGAATTTTGGATCTTGA
TGATAAGAACAAGACAGGGTGAATAAGTTCATTTAATTAAGATGGAAAGTGCGAGTTTAACTTGAGTTAC
GTGTAAGGTTTCATAATCAAGTGTACATATGTATATGTATTAGGGTAGATTAATGATATTAGCTATCAAA
TTTAATAAAATGTATATTTAATATTATTTTTTTATCAACAGTAAATTTTGTTAATTTAACAGTTGAATTT
AAAGTTTTCATAAAATAAATTAAACCCCACATTATTTCAAAAAGTAATTAATACTTTGTTACTACACTCT
TAATTATATGCATAATGCATTATATTTTGTAATAAAAACTTTATATTTACACACGTATGACCATTGGTGA
ACCTACACTGTGGCAAGTACACCCTCATTTTCTAACATTCACAAATAAAAGTTTTTCTAAACAGAAAATT
ATAATAAAATCTTATAATTTTATATTTTATTTCATTTATATTTATATATTTATGATAAATTCCTATATTT
ATATATTTAAACCCACTCATTTTACTTTTTATAATTTATTCACATTGATTCAAGTTCTAAATCTACACCC
ATCGAGTGCATAAATCAACTGGCATATATTTTAACTTAATCAAAGGTCTTGAGTTTAAGTTTTGAATATA
AAATTACCTTATATATTTAAAGGAAGAGTTTGTTATCCATGATGATTCCATAAGACTCTCTAACAAAATT
ACTTCCAATAAAATATACATGTGGTTTATAAAAAAAAAATTCCATCAAAATTTTACAAAAACAATACAAA
AAGAATAAAAATATTTTTTTAAAAAATTAATTCATTTATTTTGAATACATTACTTACTTTTATATATATA
TATCAACAGGGACATAGTAATTCAAGACTATTAATGTTGTTCACCCGTGACACATGTCAACTCAATATTA
CACAATCATTATCAAATTTAATTTTAGAAAATTTAATATTTTTTCCCATTAGCATATAGTCATTTTTATT
GGAAAATACATTGATGAAACATATTATACTAATTAAAGGATAAACATTATAATTTATAAAAGCATTCAAC
TATATCCATTAATTGTAAAGAAAATTTTCAATTGAGAATCGAAGTTAATAATTATCAAAATAATTCTTGC
TTTTATTTATGAAAATATATTGTGTGATTCTTAATTATTTTCATAAATATATAAAAATGAATATCATCAT
ATATTTTGAAGTAACTTAAAATATATTTAATCCTAAGGTTCTACATGCTTGAACAAACGTCTTCATCACA
AATCTTTGTAGAAAAAGTAAATAAGACACTACCAAAAAAAAAAAAAATCACCACCACTACAAATAAAAAA
GGTACGCAAAAAGAGAGCTTACACTATTACCACCCTACACACTGTCTTTTATCCACATATTCCTTCTCAA
TCGGTAAAAGAACCAATAGCTATGATAGACATCCCCGGCCGGACTCGATATTTTTTCAAATGTTCCCTCA
AATCACTGTTAGTTTTGATGTTAAAACAATTTGTTTCTTGGTTTTGCTAGTGAACCGCTTGATTTCATAT
AGCAAAATAAGTTCCTTTTTTTTTTTTTTTGTAGGCTAGAAAAATAAGTTGCAGTAGATAAAAATAAAGA
CAAAGCATTCTGATCGCTATAATTGTAACCAATGTGCAATATTAAAGGGGTGTCTGAGAGCATACAATAT
CATTTTGTAGCCTTTTATACCCATTTCACTTAATTTGCCCATGTTCTCTGTCCACTCGTTTGATGTCTTC
TAAGTAATAACTATCAGTTTCATTGACCTTGTGGTCATAACTCATAACTACCATCCTTGAGCTAACACAA
AGAATAAAGAGATATTTAGGAAGATAAAATTGTGCGAAAGTAAGAAACATTCAATTGTAATATGCTTCAA
CAATAGTATGGCCAACAGTAGTGGCGAATCTAAGACTCTGACTAAGCAGCCATAAATTAAAGAAGCTTAT
TTACAACTAGTGTTATCGGAGAATGAAAAATTGAAGAATAATAAGTTCAGCTATAATAAACTCGAGGGAG
GAAAAACAAAGAAATTCATGATAAATAGATATAACTTATTAAATTTAAGGGGTGTATTTGCACACCCTGA
ATTATAGAGATTCTTATATCTTTGAGAAAATAATTAAATTGGGAAAAAAGAGATAATGACTGATTGAGAT
TTGCCTCAGAATTGTTCGTTTTAATATTGGTACGAATCTAATGGTTTTATCCTGAAAGATGCTCACAAGT
ATTGAGGGACTAATAAATTGTTTATAAACTACTACTAAATGAGATGAGACTTTAAGGTGTACTGAAGCAA
TATCATTTAAAAAATGACTACTCGTATTTGTGTTGAGAAAATTTATTTTCAATGAAAAGAAAATATATAC
ATATAAGATAAAGTAATTAACATAACGAAAGGAAATAAAATGCAACATTATAAAAACTACAACTATATAA
ATGATATATACAACTCCTAGCACATGCATTGGATTGTGAATTAATTAAAATGTTGTATGGATGGTAAAAA
TTCAAAACTAAACCCCACACAATTTAGTGACACAGAATATAATTAGCGTTGTTCTTTTTACAGAAAACGA
CGAGAACAAAGGTGTCAAAGGAAAGGAGATGGATGCATGTGGTATGAGCTCATCCAATTCCAAACATGTT
GTGGACCAAAAGCGAAGTACCATGAACATGATGATCACGACGATTCTTCTCAGATTTTGGGACCGCTATG
ATATGAATTGCGACTACACTACTAACTCTTACGAACCGGGGTCATCATAAAACCATTACCATTTACCACT
CTTTTGAACGTTAATGTAGCCTAAATCTTATATCCAGAGAACCAGACCCTGTTTACATTTCCTTTTTAAA
ACGTTTCTGATAAATTTCTCTTGCTAGTGTCTCAGAACCCAGTTAGCTCCTTCCTCACCACGTGACACTT
CAGTGAAACTTGGAGATGCCAGCAGGTTTATTTCAGCCAGGGTCTTTGTCTCTCAGGGCAATTCATTAAT
TTAAAAAATAACATTTTTTTATACATATTCATCAGTGCACGAGGAGGAGGGATAGTATGTATCACACTTT
TTAATTCACTTTCTATTGTTTTCTGTTAGTTGAAATTCAAATATCCCTCACTAATTTGAGACTGAAACAT
TTCACCAAAAAAAAAAATTGAGGATGGAACTTTCTTTTTTAGTTGATCATAAATTTTTTCTTCTAAAATA
TATAATGTGGATACATATTTTTTGAGATTGAAACCTAACAAATGATAAATAAGACTCACTTATTTAGTGA
GACATACATGAATTTCAGAGAATATTTTCCTATATAGGTTATTAGCATTTCTTTTAATATTTTTTTTTAT
TGTCTTGTTTTTAAAAAGTTGGCATTCTTTTTAAAATTGACTTTTTTGAGATATTGAACTATTTTAATAA
TAATAATAAAATTAAGTTATATAGTGTATTAAAAAGAATAAGATAAAATGTGTTTTAAATTTCTCAAGAT
TTTAGTCAAAATTAGTTTCAGTCTCCTCTATTAAAAATGTGTTTTAATTCTCATATTTTTAAAGATATGG
TGAATTTCATTTTTAATCTTGAACAGTTCTTTAATTTTGACTTAATTAAATTCAACATATTTCAGAAACA
CGGGAACCAAAACCACCATTTTTAGAATCCAAGACTAAAGATCTTAATGACGTAAAACACAATTTACCCG
TGAGAATATTAAAGCTAGTAGTATTGCTTTTCAGTGTGTTTCCTACGGCACATTGTTGTGTGTGGAAGTG
GAAGCTAGAAAACAAAGGCAGCAGAAGAAGTATGGTCCTACAAAGTGTGTAGTAGTGAAGAAGAAATAGC
CGTTGGTGGTGGAGAGGCGCGGGTTTGCAATAAAAGAACAGCGCGCCATGATCCTATAATAAACCCTGTC
AACAAAAACAAGTATGCTTCATGAATAGTTACTATTTACAAGGAAAACTAGCCGTTACTCACTTTTTCTT
CTTTTTTTTTTTTTTGTAACAAATTCTGAACCCTGCATGTTCATTCTCTCTCTCTCACGCTCGCAACCCG
CGCGCGCACCTACACTTCTTTTATGTCATCACGTGCTCCTTCTCACTCTCCCTCTCTCTCACTACAAAAA
CCATTCTTCAACTTGCAACACACGCACACACACACTCACACACACTGTTTTTTTGTTCCACTAAATCAAA
ACCTCTTATCTCTTACTCTCATTACATTCATTCTTTTGATTTTCGTTATGGTAGTAGCAGTGGAGAAAAC
CAACCTCACTTCACAATCACAATGCTTCAACCGTGTTTCTGACAAGAAGAAAGAAAGATGCAAGACACAC
ATGAACAACGTTAACCCATGTTGTTTTTTGTTTCTCTTATGTGTGTGGAGCCTTGTTGTGCTCCCCTCAT
GCGTGAGGCCAGTTTTGTGTGAAGATGAAGGTTGGGATGGAGTGGTTGTGACAGCATCAAACCTCTTAGC
ACTTGAAGCTTTCAAGCAAGAGTTGGCTGATCCAGAAGGGTTCTTGCGGAGCTGGAATGACAGTGGCTAT
GGAGCTTGTTCCGGAGGTTGGGTTGGAATCAAGTGTGCTCAGGGACAGGTTATTGTGATCCAGCTTCCTT
GGAAGGGTTTGAGGGGTCGAATCACCGACAAAATTGGCCAACTTCAAGGCCTCAGGAAGCTTAGTCTTCA
TGATAACCAAATTGGTGGTTCAATCCCTTCAACTTTGGGACTTCTTCCCAACCTTAGAGGGGTTCAGTTA
TTCAACAATAGGCTTACAGGTTCCATACCTCTTTCTTTAGGTTTCTGCCCTTTGCTTCAGTCTCTTGACC
TCAGCAACAACTTGCTCACAGGAGCAATCCCTTATAGTCTTGCTAATTCCACTAAGCTTTATTGGCTTAA
CTTGAGTTTCAACTCCTTCTCTGGTCCTTTACCAGCTAGCCTAACTCACTCATTTTCTCTCACTTTTCTT
TCTCTTCAAAATAACAATCTTTCTGGCTCCCTTCCTAACTCTTGGGGTGGGAATTCCAAGAATGGCTTCT
TTAGGCTTCAAAATTTGATCCTAGATCATAACTTTTTCACTGGTGACGTTCCTGCTTCTTTGGGTAGCTT
AAGAGAGCTCAATGAGATTTCCCTTAGTCATAATAAGTTTAGTGGAGCTATACCAAATGAAATAGGAACC
CTTTCTAGGCTTAAGACACTTGACATTTCTAATAATGCCTTGAATGGGAACTTGCCTGCTACCCTCTCTA
ATTTATCCTCACTTACACTGCTGAATGCAGAGAACAACCTCCTTGACAATCAAATCCCTCAAAGTTTAGG
TAGATTGCGTAATCTTTCTGTTCTGATTTTGAGTAGAAACCAATTTAGTGGACATATTCCTTCAAGCATT
GCAAACATTTCCTCGCTTAGGCAGCTTGATTTGTCACTGAATAATTTCAGTGGAGAAATTCCAGTCTCCT
TTGACAGTCAGCGCAGTCTAAATCTCTTCAATGTTTCCTACAATAGCCTCTCAGGTTCTGTCCCCCCTCT
GCTTGCCAAGAAATTTAACTCAAGCTCATTTGTGGGAAATATTCAACTATGTGGGTACAGCCCTTCAACC
CCATGTCTTTCCCAAGCTCCATCACAAGGAGTCATTGCCCCACCTCCTGAAGTGTCAAAACATCACCATC
ATAGGAAGCTAAGCACCAAAGACATAATTCTCATAGTAGCAGGAGTTCTCCTCGTAGTCCTGATTATACT
TTGTTGTGTCCTGCTTTTCTGCCTGATCAGAAAGAGATCAACATCTAAGGCCGGGAACGGCCAAGCCACC
GAGGGTAGAGCGGCCACTATGAGGACAGAAAAAGGAGTCCCTCCAGTTGCTGGTGGTGATGTTGAAGCAG
GTGGGGAGGCTGGAGGGAAACTAGTCCATTTTGATGGACCAATGGCTTTTACAGCTGATGATCTCTTGTG
TGCAACAGCTGAGATCATGGGAAAGAGCACCTATGGAACTGTTTATAAGGCTATTTTGGAGGATGGAAGT
CAAGTTGCAGTAAAGAGATTGAGGGAAAAGATCACTAAAGGTCATAGAGAATTTGAATCAGAAGTCAGTG
TTCTAGGAAAAATTAGACACCCCAATGTTTTGGCTCTGAGGGCCTATTACTTGGGACCCAAAGGGGAAAA
GCTTCTGGTTTTTGATTACATGTCTAAAGGAAGTCTTGCTTCTTTCCTACATGGTAAGTTTCGTGTGCTG
TTCTTTCATTAAGTGTTGTGTGTGCTGTTCTTTAATTATAATTTGGAGTTTTACCTTAGTAATCTGTATA
ATTCTAATCGGAGAACAGTACAAACAAAAACACCTAAGGAACAACACCTTAGCTTTAATATACCATATCA
ATAAGTGAATTATTTTCTTGTTCATCTTGATGCAGGTGGTGGAACTGAAACATTCATTGATTGGCCAACA
AGGATGAAAATAGCACAAGACTTGGCCCGTGGCTTGTTCTGCCTTCATTCCCAGGAGAACATCATACATG
GGAACCTCACATCCAGCAATGTGTTGCTTGATGAGAATACAAATGCTAAAATTGCAGATTTTGGTCTTTC
TCGGTTGATGTCAACTGCTGCTAATTCCAACGTGATAGCTACAGCTGGAGCATTGGGATACCGGGCACCT
GAGCTCTCAAAGCTCAAGAAAGCAAACACTAAAACTGATATCTACAGTCTTGGTGTTATCTTGTTAGAAC
TCCTAACGAGGAAATCACCTGGGGTGTCTATGAATGGACTAGATTTGCCTCAGTGGGTTGCCTCAGTTGT
CAAAGAGGAGTGGACAAATGAGGTTTTTGATGCAGACTTGATGAGAGATGCATCCACAGTTGGCGACGAG
TTGCTAAACACGTTGAAGCTCGCTTTGCACTGTGTTGATCCTTCTCCATCAGCACGACCAGAAGTTCATC
AAGTTCTCCAGCAGCTGGAAGAGATTAGACCAGAGAGATCAGTCACAGCCAGTCCCGGGGACGATATCGT
ATAGCACAAATTTTGCATTGATTTTTTTGTGCCAAATGTAGTAGGCCTACTATATATATGTTCTATGATT
CTTTCATTCTTATATTATTTTTGCCTGTTTGAATGCTTGAATTTGTACATACTCATACTACAATAAGGTG
TAGTTCTGGTTAATTTTACCTCTACCTCAAAGCTGGGGTGTAATTCTGTTTCCTCCAAGGCACATAATAG
TTGAAAATAGTTCTCAGGAGCATTCATTGTTTATTCTGCAAGATTCTCTTTCACGGCTGCTATCTTCTAT
GCATGCCCTGCCCATAAATGCATTATGAAGAATTGTAACGGCTGTGTTTTTGGACTTCTTCAAAAAGTTT
ATGTTATTGCCAGGTGTATATATCAACATGTTTTAAAGATTTTCAAACAATCAGGTTTTAGATGTGGGTT
TGCATGCATGAGATTGGACTAGTGCGCTTGATGTAGTATAAAATATAAATTGTCCAATCAGCACCCTCTA
CATGTCCAAATAATGGGCCTTATGAAACTTAATTTTTTAATTACAAACTACAGTAATCTTTTTGAATAAA
GATTTACAAATTACAACAGACATGTGAAGTCGTCATCTTTCATTGCCAATTCTTTCAAGTTTACTACTAT
TATTTTCCTGCAAGCATTCCACATTCACATCTGATAACTATGACAGCATCTCCCAAGATAATGACTTCCA
AGTTCCAACACTGGCTCTGTACATTTGAACTAATTTTATATCATTTATCTATTGTGATTGAAATATAAAA
TTGAAGTGATGTGAACAATACGAATCACATCTTGAATTAAAATATCTAACAACGGGAACAAATAAGAGGC
CCAGAAAAAAGGGATAAATAACGGATAACAAGAAAGAAAGAAAAAAAAACCCAACATAATTCCAACTTCA
AAATTCACTCAATAAAAAGTTTAACATGTAAATTTACTTGGAAACAAAACTCATAAGCAAAGAAAGTCAA
AGTATACATAACCAATAATAATAATAAAAGAAATCAGCTTTATAGCATTAATTTGGGATGCTCTGCTTGT
ATGCAAATGACACAACCTTACCCTCAAGATTGCAAAACACAGATGAGTAACAGATGCAATGTGAATCAAT
AAAAAGTATTGTTGCGTTGTTGATGACACAACCTTACTCATAAAAAATGCATTGTTGATGGCTAGCATTG
TTGCAAGGTATTCATACAGTTTATTCTGCAACATAGAGAAAATACAACTCATCAACACCAGGAAATGGTT
CTGTTCAAAATCACGGATTATAAAAAGTTATTATCTAAATGTTACAAGCTTAAGAAGATCTATCATTGTG
AAAGTCTCTTATGCACATTAATATTACAAGCTTAAGAATGCTATACAAATGTTTGAGGTTTTGATATTTA
ACTTTTTATGATATGCTTTGATTTAATAGTTGCAAATTGCCACATTTCTCATGTCAGTTACTCGTATTCT
CCCATAAATAAATAAGGCTTCTTCTGTCTCAATTTATTTTACTTCTAAAGCAACAATTTCTTTCTTTCTC
ATTTTTTTTGTACCCTCTGATCAGATCGTAGTCCGATCCTCAAGCCTTAGCCTCTACACACTTTGTGCTC
GAGGCTCGATGATTGTGTATGCTTCTGACCGGACTGTAGTCTAATCTGGTGGTGGATCCCAATCTGATCT
GATAACCTCCACAGTATTGTGCACATTATAACACTGACGTAGGGTTAACCATGTACCGAGATCCTTGGGC
ACAGCATCTTGATACGCTCAATTGGCCTCGATTCCTGAATATCGGAGATGGGAGAGTAGGCAGGTCGCAA
TAAAAGGTCATACCATTAACGTAGGATGGTAGGTTAAGTAATAATACCCTTGACTTGATGCTCTCAACTA
GGTCACCTTGACATACGACAAAGCCTTTTCTAAAACAATTTTATACATTGAAATTTGGAAATATGTATGT
ATTGAAATTCACAACAATACTTTTCTAACTCATATCCAAATTTAAGATCAGCCTAAAAGCCCAAATTAGG
GGATGCAAAGAATTCTATCGTTAAATCAAACACATCAGTCAAAAAAGAAAACATACATAGGCACAATCAA
CTGATCAAGTATAACAGTATTTCAGATATTGTTAGCGACTAGAAGGTTTGTATTTAATTTGCAAAATTTA
ATATGAATACTACTGGTTCTATTTATTTTATAAACTGGTATTAGTAATGAAACTTTAAAATGGGGAATAT
TTTAGCAAAAAATAAAGGTTAAAATATGTCTATGATTCTTAATGAATATTCCAATTTTATGTTTGCTTTT
TAGTATAAAAAAATTCCGTTTTTGTTCCTTGATAAAAAAGGAAATTTGTTTTTGGTTTTAAACACTTTTT
TTAGTCCCTATTAAATTACGAATTTCCTATTTATTCTTTGATAATTTTTTGTTTATTATTAGTCCCTTCA
AAAATTACTAATAATAAATATTTTTTAACAGGAAATAAATACAAAATTCTCTAATTTATTAGGAATTAAA
AAAATGCCAAGGACAAAAAAATTATTATTTTATTAAGAATTCAATGCAACAAAAAATTTACCTAAAAGCA
AATAAAAAATTTGAGTATTTCTTAGGAATTAAAAATATTTTAATAAAAATAAAATAAAGATCCAAATGAT
AGTGTGATAACCGAAGAGGAATGTCTTTCAACCACTGCCTGACCGCCACCACTGCCAACAGCCTAGTATC
AACCGAATCCACATATACCAACAATCTTCAGACAAACACTTCTAAGTTGGTGCTGAAGAGACAATATCTC
ATGGGTAGATCAAATTAAGAGTGCTACCAATAACAAAATCGGGATCATTTGACTAACAAACAGTTATGTG
CATTGGATGTTCTACCATAGTACATTGCTTTATGTGAAATTCTTTTAATTATTCAATATTGACATGTTCT
TATATATATATATATATATATATATATATATATATATATACGAGGGATTGTATTATCTCTGAAAAAAGAT
TTTATCATAAAATCATAATGATTTCTCATAATGTATCTTTACATTTTAAAGTTAGATAAATAAAATTGAT
TTTAAATTGTTAGATATAATTAAAATACATAATTAATATGACTTTTAACAAATTGATATATAAACACTTA
AAAAAAAGTTTCATGACGTACGGTGTGTATTGTTGGTACAAAAAAAATTTATACTATCAACTAATTAAAA
TTATTATAAATAATAAAATTAATAAAAATTACTATAATAATCTGTAATTAGATTATTGTAAAATTGTTTT
ATAATATAAATATACAGTCTTTTTTCTTTAAGAAAAATTGCTAGACCAAGCAATATGGACCATGTGCTTT
CTGAAAATATATAACACAAAAATTCCATTAAGTTTTTTTGCACCTATAAGCTACATCCGCTACGTACTGC
ATGTGGAGCCTCATGAGTGTGAGGATCTTCCACAGGTCACTAGTTTGACATCTGAAAGCTCCTCGTGTAA
AACGTGAAAACAAATAACAAGCTTGGACTGGTGTACGATTTAGTGTTACTAGCTATCCCATGTAATAAAT
ATATAAATCTTGAATCACAAGGAATGATGCAATATATGGTTCCTCTAATAGTAAGTTATCCCACCAAATC
TGAATATAATTAAGAAGTTGTATTCGTCTGAATGTTGTGTCTAAAAGGGTTGATTGATGAATGATGGCTA
CATGTGAGAGTTTGATAACAACAGCTAGCTAGCCATTAGCCAAGCCACTAACTAGACATTAGTTTTGGTT
GGTTGTCAGACAAACCGTTAGACCTGAGAACGAAAGCGTATTAAACAAAAGATGATATGTAGACTTTTAA
TATAAAAAGAGATGGAGAAACCAAATTGAGATTTGATAGGTGAACTATAAATCATGACAGTGCATTAGAC
AAGTTGGTAGAGTTTGTTACTAACTCATCAGATTCTTAAGAAAGGCAAAAATAGAAACTACACCACATGT
CGCTAGCGATAACGTGCAATTTATAAATAAATAATGGCTTCATTTTCATGGTTAGTTATAAATTAATGGG
TCACAATTCTTAATTTATTAGGAACGTATACTTCATTTTGAGAGTGTATAAAGTTGGAAGAAGAAAAGGG
ATATAGAAAGAATAAAAAAATGGATTTATCTAATTCATCGTAAATGAAAATGAGATTAAATCATTCAATC
TTCATTGAATAATAGAATTTAAAAAATTGTCTTATTCTGAATTGTATCATTAATATTATAACTATCATAT
TTAATGTATTATCTTTCTTATCATTTATGTATAAAATTAAAAATTTATAATTAAAATTATATTAAAATAC
ATAAATATATGTAATAGAATTATAAAAATTAAAATTACAGCTATATATAATTTCCTGTCACTTAGACTTG
CAGTAGACAATTGTTTGTAGTTAAAAAATATGAACTGTAGTCCGGCTACGACTATTAATTTCAAGACTTA
TTTAACAGTTAATAATAATTTTTTGTCATCTCGTCCCCATTTGTCTTGCGTGTTAATATATGTTATAAAT
AGACATTGATATATTTTTTTATGTGTATTTGTTGATACATAACAACAATAGGATTGTAATTCAGATTTTA
ATATTTTATTACATGTTTATGTATTTTACTATAATTTTAATTAACAATTATAAAGTTTTAATTTTATACA
GAGAAATTAAAAATGATACATTAAATGTAATAATCATAATATTAATGGACAGAATTCATAATTAGACAAA
TGTTTAAATTGTATTGGTTATTGAAGATGAGATTTTTTATTTTCATTTTTACTTGGGGTCTTAGACAAAC
CTAAAAAAAAGAGAAAATAACACATGTAATAATTAAGTGAAGTAAATATAATGGAAAAAGATGACGAAAT
TAAGATGAGAAGAAAATGCTATGAGATTGAAAAGATATTAAGTGTCTCTTTATATAAAACTCAATTAATT
ACGAGTTAACTGGTCATGCATGAAAGTGTAAAACATTTTTATGTTATCATTTAATTATAAATTATAGTTA
ATATAATTTTTAAAATAATTATCATAAAAATTAATAAATTTACTGTACAAGATGAATTTTAATTAAATAG
TGATATAATTTTTTTTTACACTAACAAGTGTATAATTCTTTATCTCTTTAATTATTATCTAACTTTTGAT
TCCACGGCATGTCAATATTTTCTCTCTGACCAAAATAACTATCAAGGTTAGTAAACGAATATAAAGACAA
ATCCATCATGTTCTTTTGTGTCAAAATGAGGCCTTCTTAAAGATCACGCTCAATGATATGTAGTTTTCTA
AGTCGCTAAAATGCATGTTACCCTCATGAAGCTATAATAGGGTTCAGAGATAGCTTTAGAAGTTCAATAG
AGCATGTGGACCTGGGAGTGAGGTCGTATGTCGCTATAATGCTGTATAAACTTTTGGTGAGCATGCATGA
CCATTTTACTACTGGGGCTTCCATAGTGGGTTTCAGTGATAGTCTTCATAAGTTCATTAGTCTTTACAAG
TTCAATAGAGAGAATATATGGGGACCTCGGAGTGAGGAGGTTGAAAGTCACTATAATGCTGTATAAACTT
TGGTGTGTATATGCACCATCTGATGGCCATCCAATGTCCCCTAGGGACAACAGGGTACCTAATTAATTGG
TACCACAACGGGGAGAAAATCAACACGTTTGTGGAATATACATACCTAGAATTGAAGGGCTAGCTCAATC
AAGCTAAACTTGAATTCAACTATAGAAATTAAATTAAATTGAAATTTGGTTACACGAGTCAGGACCATTA
GTTATAATTAAAATGCGTTAGCACAATTTCTAACGCTATAGGCATAGAAGCACTAATGGTGACACACACT
AGTATAAAAATACTTTTAATATCAGTTATTTTAGATTTTTTTGTTTGTGTAAGTCAATCAATTTTAAAAG
TTACTTCTAAATCAACTTTAACAAAAACTAATGTAGAAATGATCTAGAAAACTTTTTTTTTAAGTTCTAA
CTCTTTTTCATCAATGTTATACATATATATATATATATATATATATATATATATATATATATATATATAT
ATATCCCAAAATAACCAATCAAATAAACTACTTAGTTTACTTATATGTTAAATCATCGACCTATTACAAG
AGAGGAGGTCTAACTCAGATGATTTATTATAATGTTAAGTTATTTTAAATCTTTTAATATTTTTATTTGA
TTTCTATGAATAAAAAAAAAATCAACTTACCCAACTTTAAAGTCTCAAGTCATTGGATATTATCTTTTTT
AAATATATATATATATATATATATATATATATATATATATATATATATAAATTGAACATTTGTTGTTATA
TTAGCTAATATTGAACATTTATTTATATCTTAAATAACATATTATCTATCAAAATAAAATGTTATAACAA
ATAATAATTCATTTTTATTTGTATGAATTTTCATAAATTTATTTATTTTCAAAAACTTTGAACTCAATGA
TTGTATGAAATAATTTATATTCTTAATTAATGAATTGATCGTATCCTATATATGCATAGTATATGAAAAA
TCAATTCTCTTAAAGTAACATAAAGAGGCCCTTTCGTTTAAGTAATTGAATTACTTAGACTTCAAAAAAA
ACAATTGAGGGGAATCAAACAATTAACAGATAATTAATTCTAGCAAACTATATTCTCCTATATATTGTCA
AATATTTGAAAATTAAAACTGTAAGTTATGATAATGATATTATAGTTGAACACGTGGCAGTATGGTGACC
CAATGTTCGTCAAAAATCAAAATGATGTGTGCCTGGTACAGTCAAGTACGTATCTTTGAGTATGACATTC
TTGTGCATGTAATTCTTTGCTGATCTTATCCATGTGTAAAATAAAATTAGTTATGTTGGATGACAGGCCA
CCTAGTTTAATACTTGAGAAATATTTTTCATAAGTATCTCTAACTAAACTCTTGTTAGATGCAATCAAAT
CACTTTAGTTACTTATAGTACTGTTATTTTATTTGCTCACGCATCATGGAAGTCTACGGTACTTATAGTA
CTGTTATTTTATTAGCCTAATTATCCATGTATAAGAATCATTGAATAAATAGTTAGTTTTACCAGATAGA
AAATAAAAGAGGGTAAGGAACACCCAACCTATCATGAGAGCTAAAGCTTCACAACAAGCAACGAACAGCT
TTTAACCTTAAACTAGGCTAATGCCAATATTAAAGAAGAAATAATTAAAATTGTAAGGCTGGTCGTGTAT
AAATTAAACAAAAGGCCCTCTATTCAAACCTTCATATATCATACCTGTTTTTAATTAACGCGGACTACTT
TTTCATATAAAAAAAAGATCATTAGAGGATTAATTTAAAGCGTTTTAGTTTTTAATTACCAAAGAGTATA
ATTATTATTAGGCGCTTTGTCCCACAATCAATCACCTAAACAAGAAAAAGAAAAAGAAAAAAAAAGTCAA
ATTGGACTAATGCAAAAGTGGCACAATCTTTGTCTTGAACTCTTTAATTAGCAACAAATTATACTCTTCT
GCACAAATCACAAGAATACCTTACATGAAAAGAATGTTAATTTGACGGTTTACATTAAATTATATGCAGT
TTTCTGCAGGTAATTAATTTTCAAGAATTTAAGGTGGTTGGTAATTTTCAATAGCTAGCTTGACTAGCAA
AGGAAAGAATAAAGTTAAAATGCTTCTTGTTTTGGCCTTTTTGGATTGTTATACTTTTTGCTAAACGGAA
ATGGTTATATGAATGGTAAAGGAGATAAATTGTTACATAGTCTAAAATTGTTATAGTCTTAATCAATCTT
CAAACAATATATAATATAATTTTATTAACTATTTTTTATAAATTAAAATTTTCAATTATATGTCACATAA
AAAGGAATTATGACCATAAATATATAATAGTCTTAAATCATGTAATAATTTGTCAGCAAAATAAGAGATT
AAATAAAGTTAGAAGGAACACAATCAGTGGATTAATTAAAACCCGTCATATAAACTAAATTAAATATCAA
ATCTCAAAGTACGCATAAAAAATCTCAATAGATTTTTGTTAATAATTAACTAGTATTCTTAAAATATTGA
TTAAAAAATTAAAAGAAAAAATATATTTATTATATAAATCAGAAAAATATAAAAAAATTATAAATAATAC
AATTTTTTTTCTCCTAATAACAATATTTTTATTTGATGTCCTTAGAATATTGGGCTAACATTTAACATAT
AATTATACTTGGGAAAAATTTACTAGAAATAGTAATAATAGATTCTCTAACACTTTCTCCTAACATAGTC
TATGATTAATTTAAATTTATTGAAAACTATGAAGTTATGAGAGAATTATTATTTTTGTAATTTTTAAGAA
ATTTCAACTAGAGAAAATATGTTTAAAAGAGCCTGCTGTTAAACTTTCTCAAAATTTATTTTCAACCTCT
AACCGCAGACTTCTGAAATAAGCATTCATGCACTTTATTAACTAGCGGGTGCAACAACTCCTATGGGTTT
GGAACCAAGTTAAGTTTCCCTTTGGGGTCTGACCTCAACTAAATTAACCTAATCTGCCTAACCTCAAAGG
ACTTTATCTCTCCCCCACCTCTAATCCACCCTATAAAAGCACCCTCTCCCACTCTTACTTGCATTGCAAC
CTTAACCTTCAGCATTCACACTAAGGTGTTCCTTGCTCGCCAAAAGATCATGGAGCCTGCCAAAACCATT
CACAACAATGTCAAATACTCCCCCATCTTCTTAGCCATCTTTGTTCTGATCTTAGCTTCAGCATTGTCTT
CAGCAAATGCCAAAATTCACGAGCACGAGTTTGTTGTACATTCTCTCACTCTCTTTCTCTTAATTTCTCT
GGACTTATTTTATTCTTGTTTTTTTTAACTCTTTTCCGTTAGATAATAATTACCTAGCTGTTGATTGTAA
TGGAATAGGTTGAAGCAACTCCAGTGAAGAGGCTGTGCAAAACCCACAACAGCATCACCGTGAATGGACA
ATACCCGGGCCCAACGTTGGAAATCAACAATGGAGACACTTTGGTCGTCAAAGTCACTAACAAAGCTCGT
TACAATGTGACCATTCATTGGTATAATATCAAGCTAGCATCTTAACTTCATTTTAGATTATGAAGACCCT
TTGACTTAATTTTACACATCTGCTTACCACGATTTATAGACATATAGATATTAGTCTTACGAAAAAATTT
TTTCTCACTTTATAATTATACTACTCCCTTCACTCCTTTTTATATTAAAATGGAAAGAGTCTCTCTCTAC
CATGTGAAAAATAATATATAAATAAAAACATATATACTCTACCATGTGAAAAATAATATATAAATAAAAA
CATATATACACTTTTTACTTCTTGACATTCTAATTTTTTTTACCTTTCTTTCTTTCCCATGATATTTATC
ATTTATTCTCTCCTCTATTCTCATTTATCTATCTCCCAAGGTGTGTACATTCCATTAGAATGTGAAAATG
AAAAACATTCACAAGCATAATGTAAAAAAAATAATATTATTTCTCATAACCCTATATATATATACACGCC
ACATAATACGTACGAACGTAAGTGATACTATCATGAAAGTTCTTGAATGGCTTTCTTTTCAGGGTGAATA
CATATATTAGTGGATAGTGGTTTTTGTTGGTCATTGTTTCTTATTATTATGTCCTTAGGCACGGTGTTAG
GCAAATGAGAACAGGGTGGGCAGATGGACCAGAATTTGTGACTCAGTGCCCGATTCGTCCAGGAGGAAGT
TACACCTACCGTTTTACCGTTCAAGGACAAGAAGGCACACTTTGGTGGCACGCTCATAGCTCATGGTTAA
GGGCCACCGTTTACGGTGCTTTAATCATTCGTCCTAGGGAAGGAGAACCCTACCCTTTCCCCAAGCCTAA
GCACGAGACACCCATTCTTCTTGGTATTTTAATTTCCTTCTTAATTTACTCATGCATATGCATTATTTGT
AATTATAGCATTCATGGTAACATGGAGGCAACTATCTAAAAAAGAATTGAGATTATTTAAAAACTAATAA
GTGATTGTGATAGTTGTGATTAATTAATTAATACTATTGAAGCAAAGAGACAATATATATAGAAATTGTG
GTTTTCTGTTGTTTAATTTTGCTTTTGGACAAAGATTAAACGGTTAAAGTGATGATGGTGATGATTTAGG
GGAATGGTGGGACGCAAACCCTATTGATGTTGTGAGGCAGGCCACACGAACTGGGGGAGCCCCAAACGTG
TCTGATGCATACACTATCAATGGTCAACCTGGTGATCTTTACAAGTGCTCCAGCAAAGGTTTGATTAATT
GCTTCTTAATTTCGATTGCATTAATTGAACATGTCACATGTCTTGTTTAAATAAATTTACTTTGCAAAAT
ATTTGACATAATTAAAACAGGATATGCAGTCATAAAAAAGAGAAAACGACATATGATATGAAATTATTAA
AGATGTCAATTATTTATGAAACAAGTCAACAATAGGTTGCCTTTTGGTACAGCGTCTATTTCATGGCCTT
TCTACTTTTTGTTCTCTTTTGAAGACAAAAGTGTCTTCCCCACCAAATAAAAAGAAAAAAAAATGCAGAA
GACTTTTAAGTAAATATATAGTTTATAAATTGCAAGTTTTAGCAAGAATTTTTTAAAAATATAATTTGAT
ATTTTTTTCTTAAATTAGAAAGAAAAGGCTAACACTTTTTCTTAAAAATAATTATATTATAAAATTGTCT
ATTTAAAAAAACAAACCATTGAAATGACTTAAAGCGAAGAGATTTTATACGCGAAACCTGCTTTTAATGA
TTTTATGCAACCAACAAGGTTGCCTGCAAGTCAAATGGAAAAAAGGCATTTAAAAACATAAAGTTAATCA
AACTTTTCATTTCTTTAATTTAGATGATGTATCATTTTAATTTCTTACATTTTCTTAAAATATTAATTTT
ATGCATTTTCAACATAACTTTTTTTATATATATTCAACTAATCAGAAAATATGATAAATATAATTTTAAA
ATAATTATTGTAAAAATATTTTTTTACTATAAATATTAATTTGTAAAAAAATCTTTACACTATAAATGAT
TTACTATTCTTTTTTTTACTTCACATGGATTCCTTCTTAAAACTTTCATTTTTTTTACTCAAAATCTGTA
AATAAATCCAATAAATTCTTTTACCCTTTTGGTTTCATGCAGACACCACCATTGTCCCAATACATGCCGG
CGAGACCAACCTTCTTCGTGTCATCAATGCTGCACTCAATCAACCTCTCTTCTTCACCGTCGCAAACCAC
AAACTCACAGTGGTTGGTGCCGACGCCTCCTACCTCAAACCCTTCACCACCAAAGTCCTCATACTGGGCC
CCGGGCAAACCACCGACGTCTTAATCACCGGCGACCAGCCACCTTCCCGCTACTACATGGCGGCGCGTGC
GTACCAATCCGCCCAAAACGCTGCCTTCGACAACACCACTACAACCGCCATACTCGAATACAAATCACCG
AATCACCACAATAAGCATTCTCACCATCGTGCCAAAGGAGTAAAGAACAAAACCAAACCTATAATGCCTC
CACTCCCTGCTTACAACGACACAAACGCAGTCACTTCCTTCAGCAAAAGCTTCAGAAGCCCTAGAAAAGT
TGAAGTACCCACTGAAATTGACCAGAGCCTCTTCTTCACTGTGGGTTTAGGTATCAAGAAGTGCCCCAAA
AACTTCGGACCAAAGAGGTGTCAGGTATTGGACTATTCACCTAATTCTATTATCATGCATCAATTTAATT
TGCATGTACGTATCTTATCTTAAGATTTCAATAAATGTCTCATATAGGAAAAATTACTTATTTATGTTTA
TAATCCCCACAAATTTTACATTTTAATCCATACTCTTAAAAATTAAGTCTAATTTAATTTCTTATTCTTT
AAAAATGACTGATATGTTCTGATACCAAAGAATTCAAATATTAAATATTTTTATTTTTTGTCTTTGTATT
CTATTTTTTCATAAATTCTAATCTTGCTAATAATTTCAATTCATATTAAGATCGGTAAATAGAAAATCTA
GAAAAAAAAACAAAAAAAGTATTTTTTTTTCATTGATTTTATTTTCAATTGATTTGTCACTAACAAACTG
ATTCCTCTTAAATCTCACAAAAGTACATGTCGATATAAATATGAGATTATAAATTCATGATATCTATTTT
CGATTTTTACATATAATGTTTTTTTTATCTTTTTTAGTTCCTAATAAGCATTTTTAAATGTCTTATGTTC
CTACTTTGCATATCAGGGACCCATTAATGGGACGAGGTTCACTGCGAGCATGAACAACGTGTCTTTCGTT
CTCCCGAACAACGTGTCCATCTTGCAGGCTCACCACCTCGGAATCCCTGGAGTGTTCACCACTGATTTTC
CGGGGAAGCCGCCGGTGAAGTTTGATTACACCGGCAATGTGAGCCGTTCGCTGTGGCAACCTGTTCCCGG
GACAAAGGCACACAAGTTGAAGTTTGGGTCGAGGGTGCAGATTGTGTTGCAGGATACTAGCATTGTCACT
CCTGAGAACCACCCTATCCATCTTCATGGGTACGATTTCTACATTGTTGCAGAGGGTTTCGGGAACTTCG
ACCCAAAGAAAGATACGGCGAAATTCAACCTTGTTGATCCACCTTTGAGAAACACAGTGGCTGTGCCTGT
AAATGGATGGGCAGTTATTCGATTTGTGGCTGATAACCCAGGTAAATAAATAGGGTCTTGTTAATGGGCG
TAACATTAGTTAGGAAACTAAATATAAAAAATATTTATTGTATATGATATAAGAGAATGTAAAAAAATTT
ATAAAAAACAACTTTTCATATTTTAATAAAAAAAATCCTTTAATTTCTTACTCATGTTCTAAGAACACAA
ATTAATATTTAACTAGTAAATATTCACTATTGAGTTTTAATTAGATAACACTCTAGAAATATTATTTATT
TATATATGAATATATACTTTCTTTCCAGTTTTCATTTGTATAATGTCCTTTTTATAAAAAGAAAGAAAGA
AGCAAACTCAATCATTTGATGGGTGTGTTAATTGTTAATTGTAGGTGCATGGCTTTTGCATTGTCACTTG
GACGTTCACATTGGATGGGGTTTGGCTACGGTGTTGTTGGTGGAGAATGGAGTTGGGAAGTTGCAATCCA
TAGAGCCTCCTCCTGTGGATCTTCCTCTTTGTTAGGATATCATTTCAAAATATTCGTTGGCCCCCAACAA
TCGGAGTTTTGCAGTTTTTCTTAGTTTGGAAGCTGGTTGATGCTTCCCTGCATTAATTTTGGGAGGGTTT
TTTGTTTTGCTTCATTGCTTTGTTTTAATTGTACGTTCTTTTTCTAGAGAGAGATAAATTGGGTTTGGAA
CCTAGGAGAGGTGGTGATGATGCGGTTTCAACGCAACTATACCATCTGAATAGTCATTGCCCAACTAGTT
AAATTGATGTTATTTTCCCCCCATAGCTCAAGTTACAAACAGATTGCAAGTTTTGAATATCAATATATCA
GCTGGCTTTTTTTTGGGTTGCAACTCTTGGTCTCTTGAATATAAATATGTTGACATGTATTAAGTTTCAA
AGTCCCCTAACCATTTGCCATCATAAACTAATACAAAATTCTCACATTAATTCACAGTATAGTGTATACA
GAGGGATGCTTATAATAGCATCAAGCATTAAAATCCCTCAACGAATGTTGTCAACATATGGATTATGGGA
TAAAAATATAACTACGCTTTTCTGATAGAGGGTGGCTTTTGGTTTTCATCAGGCTGAATGTGCAAGCTGA
TAGAGGAGTATTAAGATAGAGTAGACTATTGTAATTGAGAAGCAGAAAGCTACTGTCGCTGATCATGTGA
GTGGGTGGCAAATGTACTAGTCCAGTAGTCCTCTTGAGTTTTAGCCCAATAATCTCATCAAATGAATCTC
TGGGCCTGCTTCATTAATGCTAAAAGTTGCCTCTTCGGCCTATGCTTTTTTTTTCTTTGTTAATAAAGTG
ATGAAAATAAAGGATATATTCATAAACTTTATTTATGTTTACTTTGTATAAATTTCTTAACAAATACTTA
TTGAGAAAAAAAAATCAAACTTCTCTCATAACATAAAATTAACTTGTGCATTTCTAACTTTTTAGAAGTT
TTCTTATTTAACTTCCCTAAAAAACATTACATTTGCTACAGTAACTTGGTGACAGATTTCATGTTAGCTC
TCGCATGATTTCAATTGTATTACAACATTTGTACTCTCAGTTATCCTTAATAAAATGATATGATTTTCCT
TGCCTAAAAAAAACTTGGTGGCAGATTTTGTTGCATAGATTAATTCATCTATGTGCCTTTAAGCATTTGC
TAGTCATGATGGTCTATAAATGCAGTCTTTACATGTATATTTCTAGATTTTAACATTGTTGGGTTCTGTA
CTATCTTTTGGGGTCTTTATTTGGATTCACCTTGTGCTTAAACCTAAAGGCTATTGATCATTTTATTCTT
TTTCTTTTTGAATTTTTTTCATTAAAGTTTTTTCTTTTTATAAGTTGATTTTAATTTTTAGAAGAAACTT
AATTCATTTTCTCTCTTCTTTTTCCTCCTGATGAAAAATTATTCAAATTGACTTTTAAAAAGATTGTATC
ATATATATGTGACATTTTCTTTTTCATTATTTTTTATGTTCATGTCACATAAAATGCAAGTCTCATAAAA
TGTGGTTGTAGGACAAAAAGTGATACTATACTCCACATGGTATATATACCAAAATAAAAGTAATACACGG
AATCATGTGAAGCCTACTCAAGTAGATGTGCAAAATCTTGTGAATTCAAATTAGTTGTCTTGTTTATTCA
TTACCTTTTCAATTTTTTTAATCACCATAATTAAGGCCTTTCGAATCCCTTTAAGTGATAAAAGAAACGT
GCAATTATGCAACAAATAAATTTTCGTTATGTTACTATTTAGTCAAGGAGGAAAAAAAAGTGATAAGGGA
AGAAACAAGGGATATTTCCTGTTATAACAAACTTAAAATGGCGACTATTTTGACGACATTGCAAATACTC
ATAGTACGATATAAATTTTGAATTTAATATACAATGAATAGGCATATTCATTTTCTACCCCAAAAAAGCA
TACTCATTTATGTACATTTAATTTTCTCTCCATAGAGGAATTAATGTACAACCATGCATAAGGGATGAGC
GAAAGGGACAGATTATTGCAATCCAGAAGCATCCAAGGAAAGTTGGATAAACAAATCAATTAATATATAT
AAAAAAAAAACAAAAATGCTCCTAGTAGAAGATTAAAGGAAGAGTTGGCTATATATGGCAAACCTTTTCT
AACTGTTTTACCCTCTTCTCATCACCGCATTGCATCACCAATACGGGAACTTTTCCATTACAAAACTCAT
TGGAAGCCAACATATCCCCCAAAATTCCCAACTGATCTGCATTGTCCATGAAATTTGACATTTCTTCTTC
TACAAAATTCCCCATGCTATGTCGTTTTCCCACCATCACTAGGTCATAGTCCTTCTCCATTCCCCGAATC
GCTTTCAACACTTGTATGCAATCTTCCACCACAGCCTCATGGTAAACAACGTTAACACTATCACTACTAA
TATCATTCTTGGCAATAAACTCATCTATTAGACTCTCGTCCAACGTGCTCTCTAAATTCTCATCTTCATT
TTCTACAAGTCCATTAAACCTCGAATCTTCATTGGTAGGCAAAACAAACCGGAACAAGGTGACACGTGTG
TTGGGGCGTTCCAACATTCGAATCCCCAACGCCAATGCTTCTCTATCATCTTTTCCACCAATGAAGAAGA
TACCAACATCAAAAGACAACTTGGAGCTACTCCCACTTAGCACCGAGTATCTATCCACTAGTATCCCCAA
AGTACCCTTTGCATTAGCGAGAAAATTTGTGTTGAGGTTGCGAATGGTGCTGGCCAGGTGGCTTCCTAGG
GTTTGATCATTTTGGTGAAAGGGTATAATGAGAAGATGCACTGAGTTATCTTCGGCTAGGTTACAAACTG
CCTCATGCATGCTTCTGTACGGAGCCACGTTAACATACGAAAGAACCGTTACAGGACCACTTGAGTTGTT
GGAGTAGTTCTCAAAGGCACGTAAAATGTGGTTGGTGTTGGGGTAGTTTACGGACAAAGATTTTCGCTTG
TTTTTGTTCATGGGAAGAAGAATGGGTGTGCTTTTTCCAACGAGCTCAATGAGATGGACCACGTAGACGT
ACAAGGGGCTTTGTGTGGTAGGGTTGCATGCTTCTATTAGGGCAATCATGTTGTGCACGTGTTCGTCTGT
GTGCACACATGATACGATGTTGAACGGTGTATTTTCGGTTATGTTTTGGATTGTTTTCACACTCCCTTCT
TGTATGGTTTGCGTCTTACACACCCTACGGTGCCTGTACAAGGATTTGATCAAGGGTATGCAAATTGATG
TCATGACCACCACAGACATCACCGCTACACTGAATACTTCTGTATCAATAACCTGCAAAAAGGAACGTCA
TGACATAAATTAGATTTAATATTTCACTAACAATATGTATAGCACCCACATTAAAAAGCATGAGAATATT
GAAATTCAAATCGCTTCAAAGAAATTATCATATGACCCAGAACTATCATGTATTCTTGATTCCGATTGTC
AATTTGTATTACATGGCATATAATCAATTTTATTATTTTTTAATATATGAAAATTTTGATTATATATATC
TTGACCGAAATCGTAGACATGTAGTGATGTCAAATCATAAAATAATTCCATCAATTAAAAACTTACATGC
ATAATTAATATTTTTTAATTGAGAGACAGAAATTGAGAAATTATTAGACAATATGACATCATCACATGAT
TATGATCTTATTCAGTTTCTACATGATATATAATCATGTTTTTTTTTCTTAATTTACAAAAGAAAGAATA
ATATATATGACTTTATTATGTGTCATATAGATGTTAATAAGAATCGTGAAAATTTTAGACCACCTGATCA
TTTCTCGAGGAAAGAGCATGAAAAGGAGACAAGCGATTTGATTTCTACCTCTCATAAAATAGAAGAAAAT
ATATAAGAGAATATGCATTACCCTGAGTTTATTCATTCTACCGTAAAAGATGAGTTCGACTATACCCTTG
ACATTCAATATGAGCCCAAGCACTACGCCATGTTTGGGCTTAATATTATAGGTGGGAGAAATGAGCGCAC
ATGCAAGTACCTTCACCAAGCACCCCACAAACAAGATAGCCAGAACTACTAGAACCACCTCCCAATGCTC
ATGAATTAAGGTCAAGTCTGTCCTTGTGCCAATCAACAAGAAGAAGAAAGGCATAAAAAACTCATACACG
ATCAATTCACTTCTCTCTATGATTGTTGTGGCGAGGGGGGGTCCATTTGGCAGAACCAAACCATAGAGAA
AAGGTCCCATGACGAAATATATGCCAAATGTGTCACTAATAGCTGCCATAACCAAAGGCCCCAGAAGTAA
CAAGACAACGTATGCTTCTTTAATTGGCTTCCCAGGTGGTGTTCTCTCCAAAACAATGTTCACTAGTGGT
CGTATAATAAGAAGTAATAAAACAGCAAATCCAGTTGCACCTATCAAAAGCACGATTAGAAATCGCATGC
TGAATTTTGAGTTGAACAGGAGTTCCATCGTAGTCCATTGCAATATTTCACTGATCATGGCTGAAGATAG
AGCAATTTGGCCAAGTTCTGTGGCTACAAGGTTGAGTTCCATCAAGGTTTCGGATACAACAGCGAAACTG
CTCAAGGTGAAGATGTTCGGGAAGTGGTAGATTGACATTTGGTTTTGATTAGCATTACCGTTGGGAGAAT
ACAAAGAAAACAAGGTTACAGTGACCAAGAAAGAAGCCAAGAAGGGAAACACACCGAATCGCCAACAACG
TTTCGCTGATTTTAATGTTGTCACCACGTCCATTTTCAAACAAGTGAGGAACACGCAATATGTAGTGCCT
ATTTTGGACAACGTATTTAGAAACAGGGATTGTCTTACCGGAAAGAGGGCTCCCAATATTTCTTCATGGC
GCCCTAAGAATGTTGGCCCCAACAGAATGCCAGCCTGTAAAATTTAAACGAAATTAAGATTATTTATGGG
TATGTTGGATAGAAAAAACAAGACCAATAAATATAAAAATGATTATATATGTGTATGTTTTCTTTTTTTG
TGGGAAAAGGAACGATAAACTTCTAAAAAAACCTTAAGAAAACACGCTTGAAGAAGCGTCTGCCATAATG
CCGTTAAACAAGAAAGAATGATCAAACTTGGTCAGCTCGTATTGAACACTAGTAATTAAAATTACTAATA
AATTAACCTATATATGAGAAATGTAGAATATATTAGGAGTGAATGCTTACGATGACGCAGCAGATGAATT
TGGGTGTATTGATAGGCCTGAGGACATAATGAAGGGCTTTAGAGAGTAAACTGACTAAAATGATTTGGAA
CAATGTGACAGGAACCACAAAATCGAATGGATTATCACCCATCCACACGCCTAAAGAGCCTACAGTTCTA
TCATTCTTGAGACAAACTATCATTTGACCCGTTCTAGCGTCATTAAAGACCGTGGTAATGGAACTTTCAC
TCATTATATATATGTTTGTACCAAGCAATTAGTGTTGACAAGAAAAACAAAAACCTTTCTCTCTTGGGTT
GTTGGTGTTTGCAACAAACGCAAACACGAGCACTACCTAAGGGAGAACAAAAGTAGATAGGAGAAAAGCA
TATATGTGCATTTTTATATGCTCTAATCAAATTGGCAGATAAAGCAATTATATTTAACAAAATATGAGCT
GCGAATGTGGTGTTAGTTATTACAACGGTTCAAAAATCAATTATAGCAATGATAACTTTAGCCAAATGTA
TTCCGAGTTGGAGTTTCGAGAAATTTTGGCTAGGAAAGAAAATTGATATGCTATCACTGAGCTAAGGCCA
GTGTGTAACGAAAAACCAAGAAGAGTGAATTTCAATTTTGTCTCTTTAAGGTATAATTTTAACCAACCGT
GTCACAAATATCGCACAACAAAAGTTTGTGTACAAATGGTTTAATGAAGACAGGTGTAGTCAACGAGAAT
AACTGGTTACAACCAACAATGACATACGGAATAGTGGCAGGGATGATCGGAAAAAAAATACAGCAAATAG
GCAATAGTAAATTTTAATTTGTGCGCCGTTTCACGATAATATTTTCTTGTAGAAAAGGATTCTCCTTGCA
ATAGAGGAGTACAAAAAGATTTGTACAAACTACTACTAGCATTTAATAATAAAAAATAATTTTGATAACA
AAATATAAATTAAAATATGAAAATTCATGCATTTAAAATTTTAAGTGTTGAAATTTCCTTAAAAAAATAG
TTTGATTGATTGATATAATATTTCTTTATTTTGATATATATATATATATATATATATATATATATATATA
TATATATATATATATATATATATATATATAACGATAATTGATAATAAAATTTTACTTTTACTTTTAAATA
CATATATAAAAATTAAGGAAATAGTATGTGATTAATTTTACTACTTTTCTATAATATAGTTTAAAATAAG
AAATAAAATCAAGACACGTACTTGTTTGCTATTATTATTTAAATTAGAAAATGAAATAAATATTTTATTG
AACCTAAAGTGTCTTTAATCATTTAGCATGGAGATCATTTGGAAAAGTTGTTATAATTTAACTAGATGTC
CGTTTGAAAAAGTTTATGTGTATCTTAAAGTTATATTATTGTATAAAGTGTATGTTTCGAAGATCTTATT
AAAATAATTTATAATATATATCTATGATAAAGACTAACAATAACTAAAAAAAACTTTGCATCACATTGTT
AATCTTTTACATTAATTTAAAATATAATTCACATATTTTATTTTTTATTTATTATGAATTTTAATTATAA
TACATATTCAAAATTATTTATTTATTATAAATTTTAGTTAAATAAAATAAACATTTATTTTGCAAATTCT
ACAAGCTAATAAACTAGTATTACTAAATGCATAGAGTATGAGCAAATTCGTGTAGGTGGATTTTGTATAT
TATATATGCTTTTCGTTAAATAATTATTACTCGACTTTAGAAGGTTGAAAAAGGTCATAAAAATTTAGCT
ATTATGAATCTAAACTTTCTTTCTATTTTTCTTCCGATCCTTTTGTTTTTTCATCACATTAGTTATCGTA
TATGTTATTTTTTCTTGATTTTTTTCCTATCTCTTTATTCCTTCTACCTAATACACTAAAAATGATATAT
ATACATTTTTCCACAAAGACTCCTGCCCTTTTCTATCAGCTAAAATTATTTATGTACAAATAAAAAAGGT
ACAAACACAACATTTATTTATGAACAGATAAACGTTTTTGTGAGACATTAACTGAACCTACTCTATCAAG
CTTATTATTACTACTACTACTTATCTTCACTCCACCACACTGTGTCACTAAAACCGGAACCATCCCCATA
CAAAATTCTACTGAAGACAACATATCCCCCAATATTCCCAATGCATCAGCGTTCTCCATGAAAGTTGTCA
TTTCTTTTCCATTCAAAGATCCATCATTGTGGCGCCTTCCCACCATCACAAGATCATAGTTTCCTTCCAA
ACTATGCACTGCTTCCAACACCTCCACCCCATCGTCCACCGTAATCTCGTACCAACAAACGTTACCAATG
CCATATTTCATGCTCTTGAACTCGTCAATTAACCCCTCGTCCAACATGGTATCTTCCTCTTCCTCTTCAC
GCTCTTCTCTTGTCAAAATAATTTTACAACCACACGGTTTCTTGTTCACGATAACAAACCTAAACAAGCT
CACCCTCGTATCTGCACGCTCCGACATTCGAATTCCCAATGCCAGAGCTTCCCTATCGTGGGCCCCACCT
ATGAAGAATACACCCACGTTGAAATACATGTTGTTGTTGTTGGACGCGCCCAGCCGAGAGTGCCGGTCCA
CGAGTATCCCCAACGTGCATGGCGCATGCGCTTGAAACCTAGTATTCATCTTCCTGATGGAGGCAGCCAC
GTGTCCGACAAGGTCAATGTTGCCGTTTTCGTGAAAAGGGATGATAATGAAAGGCACCATATTGTCTTGG
GCGAGGTTGAAAATGGCGTCGTGCATGCTCTTGTAAGGTGCCACGTTGATGTAGGGAAGAACCTTGACTG
GCCCACTTGAGTTGTTGGAGTAGTTTTCGAAGGCTTGCATGATGTGGTTGGTGTTGGGGTAATTCACAGA
CAAGAATTTTCTGCGACCGTGTCTATGTTTTATGGGAAGGAGAATGGGTGCACTTTTCCCCACGAGCTCG
ATAAGGTGGACTGCGTAGACGCATATGGGGCTCTCTTGCACTGGGTTGCACTCTTCTAATAAGGCAGTGA
TGCCACGCACGTTTGCTTCATTATGTACACAACAAACAATGTGAAACTCTCTGTTTCTTGGAGTGCTTTG
GATCGTTCTCAGTTCCCCTTCGAATAAGCTTTCTGCGTGTACTCGAGGGCGATGCTTGTACAATATGTTA
ACCAAGGGTGTTACAATCGCGGTTATAAGTACCACACAGAACACCAATTGACTGAATGTATCCTCATCCA
ACATCTACAAAACGTAATAATAACATAAATTAGATTTGATGTATTGTACATACAACGCATTTTCAATGTT
TTTTTAAATGAATTAGTGCTTGAGTTTATATTTCAGTGTTATTTTAATCCCCAAAGTTATGGAAGAGTCA
AATAAATTTTGATTTTTTTATGATTTAATTCGAGTCCTTCAAATTGGCGTCTTTTTTCTCGTTTTGGTTC
CCAGTACTTAAGTCACAATAATAATAATAATAATAATAATAAAAGTTTAGGAAGAAAAATGAGAAAACTA
ATTAATTTCGGGTTTTATTTAAGGTTTTTTTAGTTTCAGAAACTAAAATGACATATAGATACAAATTTAA
AGATTAAATTCGTCATTTACTCATTTTTTATTAGACAAAATTTAGGTTTAAGCATGCATACCTTTTGTTT
CTTGAATCTAGCCAAACTTATGAGTTGAGTTATACCCTTGATGTTCAACATGAGGCCAAGTAGCGTGCCA
TGTTTAGGTCTAATGTTGTACGCTAGAGAAACCAACACACAGGCCAGCAACTTGGCCAAGTCTCCCGCGA
AGAAAACACCCTGAAGGGTCAAAAATAAGCGCCAATCCTCAAGTGCAGACAAATCCGTGTTGATGCCAAT
GTACACAAAGAAGAAGGGTAGCAAGAACTCAGTGGTAAGAACTTCACTCTTCTCCGCTAGCGTTGTCCCT
AAGGGGGGGCCACTTGGTACGACCAAACCAAAAATCAAGGGTCCCATGAGAAACGTTACGCCCATCAAGT
CTCCCACACCAGCCATCACTAGCACCCCAAGAAGTATCAACACAACATAAAGTTCCTTCACAGGTTTTCC
AACTGGGGTTCTCATAGCAATCAATTTCATGGTTGGTCGAAGAACAAAGAAGTTGAAGAAGACGAGCAAG
CACCAGTTTCCCAACAATGCGATTGATTTTTTCACATCAACATTAGATGTGAAGCTATGCATAACGATGA
AAAGCCATAGTATGATGTCGTTGATCATGGAGGAAGAGAGAGCGATCTGGCCAAGTTCTGTGGCTGTGAG
GTTCAGTTCAAGCATTGCATCAGACACGACGGGGAAGTTGCTCAACGACATTAAGCAGCTTACTGAGACA
CGCGCGATTGTTAAAGATGCAGAGGAAATTTGTTGGGGGTGATAATAGAGACATAAGAGTGCTAAGATAA
CCACAAATGAAGCGAGGAAGGGAATTACTCCAAGTCGCCACGTGCTTTTGGCTGCTCTTATGGTCATTAG
TACGTCCATTTTCAATGCAACTAAGAATACGAAATATACGGCGCCGGTTAAGGATGCCATCACTAAATAC
TCCGTCTGTCTCGGTGGAAATAAAACCTGCCAATACGTCTTGTTCCGCCCCAAAAATGTGGGGCCCAAAA
TAATGCCACCCTGCACATTTTCACTAAACACAACTTTAACATTCAAACTAAACCTATCATTATAATTTAC
AAATCATACAAGAATGAATTTCTTCTGCTAGAATTAACAGTTTCAAACCTTAAATTATACTAATAGATGG
TTAAGATTTCTACTTAAAATCATATATATCATTTGCTTTCGTTTTCAATGCTAAAATGATGTCAGTAATA
AGCAACAGTAATAATCACATTCATGATAATAGTGATAAAATGATCACCACAGTAATATCAATCACAATAG
TCAAAATAATAATAATAATGACCTTGATGTGAAAACTGCTAAAGTGAATTTTATATAAGGAAATCATTCT
CATATAGAAATGATAAAATTACTTATTATGAGAATAAAAACAATAAATTCTTATTTGAATGGTTAGATTT
AAAAAATACATCACTTCTTATTAAGTGGTCATGTGTGAACATTAAATTACCTTTAATCTTTATCATAATT
ACTCTTTTCATTCTTAAATTAAGATTTTTTTTCTAATTTCTAGATATATTAATTATTTTTTTCTTAAATA
TTCTTACTTAATTATTTTCTCATCAAATATTAATGAGATGAATAGAGAAATAAGAAAAGAATAATTTTTG
AATGATAATATAATTAATTAATTAATAAATTTAATGTGATTAATTAAATTAATTATTTTTCTTAAGACAC
ATAAATTAGTTGAAAGGTAATTGTAATAAGGGACAGACGGAGTGAAATAAATTGCTACTAATTTCATAAA
TAAAAAACCTTCAAACTATGAGCAATCGTGGTCATTGAAAACAACAAAGAAATGAAATCCTAACTTTTTA
TCATGAGATAAATTTTCTTGGCAAAATGATAGACAGACCAAAATGGTGTCTTCCATGTCTCTCCTCCTTC
CACAAACAAACGTTACCCCATACCTCAGACACTCCTCGACAACTATTGGACTTGTGGAGTCCAACATTGG
ACTCTTTGAAGAATAATTCGGAACAGCCTCAACTACCTTCAATTTGGAACTATAAACCACGTTATCACAC
TGCCTTGTCGGAGAAGTGCACCTATCCAGCTTATGGCGCTTTCGGCTGTTTTCCAGCGGCGAGACTAACA
CATTAATAGTTATGTATTTCAAGGGCAAAAGGTCCAAACTCTAGCGTGTGGAAGGGCGTGAGGTGGATTC
CATGGGAGAAGAAGTCTCGCATCATGTTGAGGAATAAATGGTGTATAAGGAGAAGGCTAAGCGTCATGTT
TGAGGAGACTAATTTCTTGAAGACAATTTCAAGAAAATCATCTTTGAATATTTGATTTTTAAGACAGTTT
TAAGAAAATCATCTTTAAACACTCAATTTTTAAGATGATTTTTATAAAATTGTCATCATACATCTTCTAT
TATTTACAAAATTATTACTGCCTAACATTTTAAGACAAATTTTTAAAATTATCTTAAAAAATACGTTGTA
AAAATTATTTTTTAGTAGTATAATCATATTTGTCACTGTTTTCATTATCATTATTATCAATATTTTTATT
GTTATCACTACCATCTTCACCTATCATTAACATCATCATTCCATCGTTGTCAACGTGAGAAGGTGGTAGC
TTGATACCGACATGATGACAATGATGACGATTTGTGATAATTGTTGCAAAAAAAGTGAAAAGAAAATAAG
GGGTTAAAGAGAGAGAGAAAATTGTAGCCTTTCAATATTTTTTTAAATTTATTAAAAGGGAAAAAAACTT
TTATAGCTAGCTTACCAGGACGTTGCAGATGAATTTGGGTGTTCTTAGAGGCCTGAGAAGAAAGTAAAGC
GTTCGAGAGACTAAGATGACGGTGAAGAGTTGGCACAACGTTACAGGAAGCACAAATTCAAATGGACGAT
CCCCAATGAAAATTCCTAAAGAGCCCACATTTCTATCGTCTTCGACACAAACTTGCCACTGCCCATGGGA
ATCCCAATAACTTGAGACAATTCCATTGCCTCGTGAAGTCGCCATATTATCTCTATATATATCCAACTAA
TCAAATTAGTATTTCTGTGTTTGTGTTTGCATGCATGATGACGCAACACAAACACGTACCCAGGGACATA
AAATGCTGAGTGGAGCACGCAATGATATGAAAAAACAATGTCTTAATTTTTTGTTGTTGAGTTATGAAGA
ACAATGTCTGAATGAAACAAATGAAAAAAGAGGAGGGGAGGGGTCTTAGAAGCTTTAGATTAGGAATCGG
AATATTCGTCAACGCCAAGATATAATAGCCTTGCATTGCATGTTGATATTTTCGTCAAAGCATGAGAGTT
AATGGTGTTACGGGGGCACTAATCTAATATTATAGCCACTCGTGTAACTTTAAAAAAATTTCTATTTTGG
CAAAATATAATATCTATATATGGATTGAGAAATTCTACTTAAAACTAGCGTACCACTAAAATTAATCTCA
TACGAAGTCAACACTTGAAAGTTTAAAGTAACTAATTAAAGGTTGCCCTAACAATCATAGGCCATAAGCC
CTTAGTGGGCCTTGTGGTGTGGGCCATTTTTTAAAAAGTATTGTAGGGACAAAAATAACCTTAATGAAAG
ATGGGAGTGAATGACTTTTACACCCTTGCTCATCTTCATGTTTTTCCATAGCTTCCAATGGTGTTGGACT
TTTGTGGTAATTCTATTTGGTGTTGGTCAGGGAATATGGAGGCAAGTGTTCTTCTTGGAAACAGTTATGG
GGTGGTAGCTCGCGTGGCTGTGGCGGGTCTTCGTGGTGGAGTGATGGGTTCGGCCATATCTCGTGGGGCT
AAAGAACAAAAACAAAAATAAAGCTCTGATGGGTTGGAAAGGTGTCAAATACACTCAGCATATTTAATAA
ATTATTATTAAAAAAAATACCAACAAAGTTGCAAGTAACATATCCTAACTCTTAATTAATAAAATGCACA
ATCTAACAGTTTATATTTCCTTTATCACTATGAACTATCTAACAAGATTCACCATCTTAAACTTCATCAT
TTGCACGAACAAAAGTATTTAATTTATTTTATCTCATAAATTTCTATGAACCTATTTTTGGAGATGTTAT
ATGGTGAACAAAAAATATTACTAATTCACTAAGTTTATGTGCAATTAATTTTTAGTGGAGTCAATTTATC
TTTAAAATGAGTCAAAGGACCTATTCTATGGCAATCAACCCTTTTCATTAGCTTCTATTGTTCTAATATA
CAAATTTTAAATCCTAGTTTTTGGTCTCAAATGTTTTTAATTAAATTTGTTAAATTATTTTTTATTTAAC
CTTACTAATCTAAACATCATGGTTATATATAAATGCAAGCATATTTATTAAGATAATATTTTAAAAAAAA
TTATATAAATGAAAATGATATAAGATGTATATGAATATATAATTATTTAAATATATATATCAGCATTATT
AATTTTTTATATTTTTAATATTAATATAAATATATTTTTATAAAAATAAAAATAAATTACAAATCGTAAT
AATATTTTTGTAAGAAAAATAATAAAGATAATAAAATTTACAAATATTATATTTTTAAAAAATTGCATAA
ATTATTTAATACATGTTATAATATTTTAAATATTCATCACGTTTACAAATATTTTCATCATGTATACTAA
TATATCTTATTTATATATTTAATCAAAATATTAAAGATGTAAAAAAAGATATAATCTTGTTTTCATGTGA
CCGATATTTTATTTCATGTACCATTTATCTTTTAGTTAATTTTTTATAATTTAAAAAATTAAATAATGTA
TAAAAACTAAAAAAAAAAGTCTCAGTAAATCCGGACTTAGGATAACTAAAACAAGTTTTTTTAGTCTTTT
AAGTAAATTTAAAAAAAATATGGTTAAATAAATTCTATTAAAGGTGACCAAATAGACTTTTTAGCATAGA
TTAATTATTAAAAGTTGTTAAATTCTCACACCAATAATATAATGACAAAAAATGTTAAATTATACTTTTA
GTTCTTTCGTATTTTTAAGATTTATTTTGTTTTTTTATCTGTCAAAAATCATTTTAATTATTTATCTTTT
AAATTTGGGTTAATTTGATCCTTTGAAAACATTGATCATTTTTAAATTTGAGATATAATTTTGAGTATTT
TGTAGTCGATTCAAGATGTAAAATTTGTGTTACTAAGTTTATCTACATAAAAATTATGAAAATCAATTGA
TGTTTGAATAAAAAAAGACTATAAACATGATAAAAACAACATGAAAAGTGAATCTAAATAAATAAAAAAA
TCTAATGTCAAATTGTTTTTTTGTTTGCTTCTAAGACTCACTCTTTTACATATTTTCCATCTTATTTATG
ACTCACTTTTATTCAAACACCGGTCATTCTTTATTTAGTTTTTAAGTAGATAAATTTAATTGAAGAAATC
CCAGCTCTTCAATCAATAAAAAAAAGGATTTAAAAATTGTGTTCCAATTTTTTTTTAAAATTATCACTGT
TTTAGAAGAACCAAATTGACCCATATTTAAAAAGTAAATGACCAAAATAAATATAAAAAATAAGTTAAAG
GACCAAAAGTTTAATTTAGTAAAAAAAATATAATTATTGACGTGCTTGATCATTATTAATATGTTTATGT
GTTATTTAAATTATAGAATTGGAAAAATATATTATATATTAATTGACTTGTTTTTAATTTTAATTGAAAC
AATTATATTGTTGGGTTTTTGTGACTTAGCAATCCTATTATATTATAAAAATAGTTTTATAATTTACTTT
ATTGTCAACTTCAATTAAAACAATATGTTTCTTTGACTTATTGTTTTAATTTCATCAAGTATATAATTGA
CATTAATTTACTTATTATTGATTTGTATTGAATCTGATACGTTTATAAAAAGAAATTTGCAGTACTTATT
TTATTAGTGATTAATATCATTTAATGTATATATATTATTAATTTGATTGATATTAATATTTGTTTAGTTG
ATATTGTTTCTTAAAATGACATTTATTTATAACTTCGCATAAAAAATTTAAATTTCAAAATGATAGTCAA
TATAAACGTGTTTTATGAAAATTGAGATAAGATATCCTTAATTTAGGAAACTTTCTTATTATATGACTTA
ATTGATTTGATTTTAAATGAAAAAATAAAATAAATATTTTTTAAAACATATATTTATACTAAAACAAAAC
TAGAGTTATCTTCTATGTAAATATATTTTTTATTATATGTTGTTAGATGAATTTGAAAAACACATAATTA
TGAATACATAGTATGATTTTAATCCTAGTTAATATATTTATAAGATGAATTTTAATTTGTATATCTGCCC
TCCATCATTAAATCATGGGTCTAACCCTGCTTTTATAAGTACTCTTGGAGCTTGAGCCCTATAACCCTAA
TGAGTATTTGGGACCCAATGACCTAAATATTTTTTTTCTTCTGAATAAACATTTTATTCATTAACCAAAA
AAAATATTGGTTTGAGTATCTGATCTTGCACTTTAATTGCAAATGTCCACTATGGTCAATCTCTCAAATG
AGTACGCACAAAATTTAAGGTTTTTTGAACTTCAACTCTCACTCTACGATTTACTCCACGCGCGGTCTAC
TTCATTTTTATTTTTCCACTCTCTTTTCTTTGGTGCAGCATTGCTTGTTGCGCCTTGAGTTTTAAGAGTA
TATCTCAATAGCTTATTTCAGGTAAAGATTGACTCCAGTTTAGGGCGCCGTGCAACATGTTTATCGGAGT
TTGATAGGAATTTTCTGTTAGGCTACATGCATATGGTAGATCAAGTTTGTTATGCATTTGAAGTTTGAAT
TCATGTTTACGTTTGAAGTTTGAGTACTTAATTTTTGCCACGTATAGAAAGAGACCAATGGATTTTGAAT
TCACCATGAATACTATTTTTTAAAAAGCAATCGGTTTTTCCTTTGTTAATGGCACTTTTTTGAACATATA
TAGGTGTGGTTAGTTATACGAACAAAAAAAAATGATTGAAAAAATCAATGCTGCTTGAGTTGTTACACAA
TATATAAAGTTACAACGTTTCCATAGTTTGCAGAAATATGTTTGGAAGAGTAAATGTTACAATTATTCCA
TTCTATCATCATTCTTAATTTTTCTCTCACTTTATTGTGGTAAAGGGGTATTGTATCCCAACATAAACAT
AAGTTGTGGATATAAAATCAGGTATATTTACATGTCAGGGAGATTGCATTGAAACTTCCAAAAATATGCA
TGATATGATTTATTATATCCATGTATATATGTCAAGAATTAGCCAGCTTATCGTGTAGACTTAATTAAGC
CTTGATCTTGAATTTGTATTTTATAGTAGTTGCTGCCTGATATTACATAGTACTGCAGGTGCACGCAATA
CAATGATGCATAGAAAAAGTCATTTACGTCAACGGAAGCATCCATTCCATGCTTGTCGTAAGTCTTTCCC
CTTCTTTTCGTTAATACATCATCACCATGCATGGCAAAATAGAAAATAAAGATCAATGGATTAGACTTTA
GAGCTTATATTGATGATTTTATGATATTGGAGAGATTGTTAATGCCCTGATCCACGTGAAAGGACATCTG
TAATTCACAGGGCATACTTCAGTGACTAGTTCTTTCATATTATGTGTACCCAATTGGCTACAATTTTTTT
TTCCTATAATCATACGGTGCTCACATTCATTAGTTTTCATTTTCGTACTATATTAACTCCCTCAAAATTT
TGTTTGAAACAGCCTTCGAGGCATATTTGTCTCGGTCATGGCGAGCTGTGGAGCTCATAAAATTTGAGTC
TGGAACTACGACCCTATATTTTGTAGATAATCACCATATGACCATTAAGAAAGGCTCCTTTTCAGACGTT
CGAGTTAGGTCAAGGAAAGCTACTTTATCAGATTGCTCCTTTTTACGAACTGGGATTGACATATGTGTTC
TCTCAGCCTCTCAGGGTAATGACAATTCAGATGAATCTAGTGCTAATCATGTAAGTTAATGCCTTTTAGT
TGTAGTTCTATATAAAATTGGATTTTTTTGTCTATCTCAGTTACTCTTTATCAATTTTTCTAGCGTGTGT
TTGATTAATCTTCATGGATGTGGTAGATTTAGTTTGTATTCTTCCTTGCTGTTGATTGCTTGATACTTAT
TAGATTTTTGGATTATTATGACATTATTGTGAAGTATCCCTAATTTTGTTGATAACTTTCTTAAATTATT
TGGTTGGATATCTTCCCTGAAATCTCTCTCTCAACAACATTTTTTTGTCTGCAAGACTCAAACTCAAAAT
TTTCCTTAAGGATTTGAGTCTAGTGTTACTCGATCTATCGGTAGATACTTATTAATTCTCTTTATATGAG
ATGATAGCCAAACAAAACATTAAGAGAAATTAAGAGCAGACTCTCTAACATACTCTTCTCTAAACAAACT
GAATTATCTTGATTTATTAATCTAAAGGATTAGTTTTTGTATGCATTTGTTAGGTGATTATTCATTTGGC
CTTTCTTGATGTTGTTTTTAGCACCAGTAGACCTGAGTTAATTCTCTCCTTTAATTCAAGAATTGTAGTC
TTTAGGACATTAAAAATCTGATCAAGTGTTTCTTTCTCTTTTTGATTTTAGGTGTGGCTTGATGCTAAAA
TAAATTCCATACAGAGAAAACCACATAATCCAGAGTGCTCATGTCAGTATTATGTAAACTTCTATGTTAA
TCAAGGTTCACTTGGTACAGAGCTGAGAACTCTTAGGAAGGAGGTTAAAGTAGTTGGAATAAATGAAATT
GCCATCCTCCAAAAGCTTGAACGTAATACTTGTCAACACAAATACTATCGATGGGAATCATCTGAAGACT
GCTCCAAAGTGCCACATACTAAATTGTTAGGAAAATTTATATCTGACCTTTCATGGTTGGTTGTTGCATC
TGCTATAAGGAAGGTTTCATTCTGTGCAAGATCTGTGGAAAACAATATTGTGTATCAAATTTTAGGGAGT
GATGCTACAACCTCTTCATTATACATGGATTCTGAAATAAGTGTTGTGAACTTTAAAGTGAACGAAGACG
GCATGCAAATGCCTGTTATTCATCTAGTTGATTTATTTGAGACTGACACCAATACAAGCGGCGATAAACA
TGATTCCCACTATGATGAAGTGCCATCATCTTATGGTTTTGAGGGCTTACGACGATCCAAACGTAGGAAC
ATACAACCTGAACGTTACTCTGATTGTGGTAATGTTTCTGAGATAAAGGTTGGTAATGTTCGAACCTGGC
CATACAAGTTAAACAAAAGGAAAGATGATGATGGTGGTGGTGAAGAGTCATTGCCATTAGCACAAGAGAA
TAGTGACAATAGTCAAAAGGTCAATGAACTGAGTTCTTGCCGGGAGATTATAGTGTACCATGGGAGGAAT
GAAACGCTGGAATTAAAGTCAGGTGAGGCCAATCAAACTCAACTTGCTAGTGTTCCTCTTCTTCAAGAAG
GTGATTCATTAGCCCTTGAGCATCATCATCTCAATGACAATGTTACTAGAAGAAGTGATGCATATTATAG
CACCCCTAAGCTTAAGAGGAAGAGATTAGTTGATCTGGAAGCTGATGTAGATTTTGATCCTGGAAGGGAA
GGCATAAATTCCAATAAAGGAGTTAGCGAGAAAAGACATGGTTCATCATGGTATTCAAGAAGCAGAAGCC
ATGCTGCAGAACACAGTTATAAAGACAGAAGCTTAAATGCAACTGCCTACAAGGAAATGATAGATTCATA
CTTGAAGGATGTCAATAGAACACCAACTACAGAAGAGCCACCTGTAATGGACCAGCGGAAGGAAATAGGC
AACTTTGGGCAAAAGAAGGAAGCAGAAATACCTGAAAGAGAGGACGAGGAACAAATCTCTGAGATCGATA
TGTTGTGGAGAGAAATGGAAATGGCACTGGCATCAAGTTATCTTGAAGAAACAGAGGTGTAACAACTGAT
TCCCTTTTCTATGTTGCATTTCTTTTACGGAGAAAATTTAGATGCAGTTCCTTAAATATTGTTGGTGTTG
TTGTTCAATCAAAATTGGAGTTTTACTTAGTTAATCTGCATAACACAAGTTTGCGTTAAATGTTAACACA
TATTATCAAGATAAAACTTCAATTCTAATTAGAGAACAACACCAATTAATACCGAAGAAATTGCCACCAA
GTTTTGTCCTTTTATTTATATCTGTATATTCTGGCTTTTTTATCTTCTTTTCTGAGGTTATTTCGGTGTA
ACTATCTCATCAGGGTTCAAATAGTGCCAATTTTGCCAAGACTACGGAAGAATCTAATCGCACTTGTCCG
CATGATTACAGATTGTCTGAAGAAATTGGAATTTATTGCTACAAATGTGGCTTTGTGAAAACCGAGATAA
AATATATTACGCCACCCTTCGTAAGTCAAGTTCAAAACCATGTTTGGTTTGATTTCTTTAATTCACTTTT
CAAAAAGCCTATGCAACTATAAACATAGTTCCTCATATTGACTATAACCTCCCAATTTGTTCAAAAACCT
GTTCATATTGGCAGATTGAAATGCAACGCTCAGTGAGGCACCAAGAGGAAAAGCAATGCAATGGAAAAGA
TACAAAGGAAAAGGCTAGTAAAGATGATGATTTCCATCTGCTCTCAACTCATGCTCCTACAGATGAACAT
AACTCTATGGAACATGATAACGTTTGGAAGTTAATTCCCCAATTTAGAGAAAAGTTGCATGACCACCAAA
AGAAGGCTTTTGAATTTCTTTGGCAAAATATTGGAGGGTCTATGGAGCCAAAACTTATGGATGCAGAATC
CAAAAGAAGAGGGGGTTGTGTGATATCTCATGCTCCTGGAGCTGGTAAAACTTTTCTCATCATTGCATTT
CTCGTTAGCTATTTAAAGCTATTCCCAGGGAAGAAGCCTCTTATCCTTGCTCCAAAAGGCACACTTTACA
CTTGGTGCAAAGAATTCAACAAGTGGGAAATTTCTATGCCAGTGTATCTGATTCATGGGCGTGGTGGAAC
TCAGAAAGATACTGAGCAAAATTCAATTGTTCTTCCTGGTTTTCCAAATCCAAATAAATATGTCAAGCAT
GTTTTGGACTGCTTGCAAAAGATAAAACTGTGGCAAGAGAAACCAAGTGTTTTGGTCATGAGCTATACTG
CATTTTTAGCATTAATGAGAGAGGGTTCAGAGTTTGCACACAGAAAATATATGGCTAAAGCATTGAGGGA
AGGTCCTGGGATCTTGATACTTGATGAAGGGCACAATCCAAGAAGCACCAAGTCTAGGTTGAGGAAAGGG
TTGATGAAACTGAAAACAGATCTAAGAATACTACTTTCCGGTACATTATTTCAGAACAATTTTTGTGAAT
ACTTCAACACACTTTGCTTGGCAAGACCAAAGTTTATCTCCGAAGTGCTTGATACATTAGACCCGATTAC
CAGAAGGAAAAGCAAAACAGTAGAAAAGGCAGGTCATTTGCTAGAATCACGAGCTAGAAAATTGTTCTTA
GATAAAATTGCTAAGAAAATTGACTCGGGTATTGGAAATGAGAGGATGCAGGGTCTAAACATGTTGAGAG
AAACCACAAATGGTTTTGTAGATGTTTATGAGAGTGAAAATTTTGATAGTGCTCCTGGTTTACAAATCTA
CACGTTGCTAATGAATACAACTGACAAGCAGCGTGAGATTTTGCCAAAACTACACACGAGAGTGGACGAG
TGCAATGGTTACCCTCTAGAGCTAGAGCTTTTGGTAACTCTTGGATCAATACATCCATGGTTGGTTAAAA
CAACCTCATGCGCAAATAAGTTTTTCACTGCAGACCAATTGAAGCAGCTAGACAAATACAAGTATGATAT
GAAAGCAGGATCAAAAGTTAAATTTGTTCTGAGCCTTGTTTTCCGTGTTATGCAGAGAGAGAAAGTACTT
ATCTTCTGCCACAACCTTGCACCTGTGAAGTTATTGATAGAGTTATTTGAGATGTTCTTCAAATGGAAAA
AAGATAGAGAAATTCTGCTGCTTAGTGGGGAACTAGACCTCTTTGAACGCGGGAAAGTGATAGATAAGTT
TGAGGAGCATGGAGGAGCATCAAAGGTACTCCTTGCTTCAATTACAGCTTGTGCTGAAGGCATTAGTTTA
ACAGCAGCTTCTAGAGTGATTTTTTTGGACTCAGAATGGAATCCATCGAAAACAAAACAGGCTATTGCAC
GGGCTTTTCGTCCTGGTCAAGAAAAAATGGTTTACGTTTATCAGCTCTTGGTAACAGGCACATTGGAGGA
AGATAAGTACAAAAGAACCACTTGGAAAGAGTGGGTTTCTAGCATGATTTTTAGTGAGGCTTTTGAGGAG
AACCTTTCACATTCGCGAGCAGTGAACATTGAAGATGATATACTGAGGGAAATGGTTGAGGAGGACAAGT
CTAAAACAATTCATATGATTCTAAAGAATGAAAAGGCTTCAACAAATTGAAGAGAGGTATGAAAACATGT
GCATAATTTATGTTTATATGTATCCTAATCCTACATTCTCCGTATTAGTGTTGTTAACAGTGTTTGCACT
AGATCACTAGAATTCTTGTCGGCATGTACCTTCAGTGTTTGTTCAAAATTTCCATATATGCATGCCACTT
TAGAGTTTTGATTGGAAAAAAAAATCCAAACACCACATAAAATTAGGCATGGCGTGTCGAAGACAGATTT
GACTCTTCTCTGCTGAAATGCAACGCAAATTCGAGTTTAGTAGAAACTTATCATCCAAAATTAAAATTGA
AAACTTTAATACAAATGCACATTTTGGAGCCATTCATGTCATCTCTTGGTCTGAGTCTTATCATTCTGTG
GATTGAATTCATGGTTTCTCTTATGACATTGTTGCCAAGTAATACTACTATATAAATTCAGATTTGGGTT
TCTGATAACCGTGGTCGTTAATACTATATATATAATACCTTGCAGGAGCTTGCGCGATACTTGAAACAGG
AGCAGGGACAGTGGAAAATAAAGGAGCCATAGCACCATCTGCTTGCTTATGTAATGTAACCCAATCTGTC
TATATTTTAATACACACCCCATTACGATAAAATTATGCTAGGGCCTAATTTGAATTGATTTCTATTTTAT
GGGAAATTTTCAACTGAAAAAAGTATTTGAATTTAATTTACAAGAAAGTCATAAATTATAATAGTTATGT
TGAATGAAAACATTTTTAAGGAGTTATTTTTCAAAGAGAACATTTTAAAATATAATTTGTATGTTAAAAA
ATATATTATAAATTTTAGTTATACGCATTGCATAAACTAAAATAATTATAAGTTTATAAATGTTAATGGA
GAAGTTAAACAAATAAATTTTAAGAAAGATAAATTTATAAATGTGTAGCATTGTCCTACGGATTTTTTCA
ACAAACACACATAGTTCTCCTTTTTTGGTAATTGATAAGTGTTATTGCATATATTATTTATATATTAAAA
TCATATAGTAATTATCTCATTTTTTTATCTTTTATTATTTATTGTGTCTTAAAACCATAAGAATTAACTT
TTGAGTTTTTATCTAAAAGATGTTAAAGTTAATGATTTTAGAATAATTTTGGTTGTATTTTGTGTAGAGT
TGTAGCAGAAGCATGAAAGAGGATTAATGAACTGAAGTGTCACACTCAACACGATCTCGCGAGTCAAAAC
CACTCAATCAAGCAAGTCATTTAGCGCAAGGAGTCACATTGAAAGACAGTTGTCACAAGCAAACGCATTA
AGCGCGCATCCTGCGCTTAGTACGTGGCCACTTGATCTATAAGAGAGTTCTAATTGACCAATTAATTAGT
GAAAACATATAAAAAGGAAAGGAAACATTTGTTTCCTTAAGAATGAAGAAACCAAAAAGAAGTAAAGAAG
AAGAAGCAAGGGAAAGCAAAGAAGCTAATATAAGGAAAATCCGTTTCTAGAGCTCTAGTAGCCAATCTGT
TTCAATCCATTTCTCTTTCATTTTCTTCCCTCTCATCTCACTTTTATATTTATAAGTCTCTCATGATAAT
GAATGACTAAAATTATCTATTGTTGGGAGTTTTTCAAACCAAACTCTCTTTAGTGTAATGATTTTAAACT
ATCTTTTAATATAATGTTGTTATTATTATTCATCCCTATGCTTATTTACATATTTATGGGAAATGTTTGT
ATACTAAAAACTTATGAAGAATATCTAAAATGAGTCATATCTAGGATAGAGTGATTTTTTTTAGCATGTT
CATGCATCTTTGCTCTGAATGCAAATCATCTAGTAATCAATCACCAAGGGATTGAGAGCGATATTAAGTG
ATTTAGATTTTTTTATTTGAGGAATCTTAGTTAGAATAGACTAGTAGATGTAGATAATAATTATGTTAAT
GTTAAATGAGAAAAATCTATTAAGATTAAATCAAGAGAAGTTTTGGCAAGCAAGAGTCCCAACACATTTC
TTAACTCATCACAATATCATCTCACAACTTTGAGCGTTTGTAGTTGCTTTGTAGTTGATTCCTTTTAACT
TATACTTTATAGTTGTTTTGTAGTTGATTCCTTTTAACTTATACTTTATAATTAATCAATGAATTAGATT
GGTGAATATTAGTTATTGATTGTTAATTTTTTGTTAGAAGGAGATCGAACCCATAATTTTTTTCTCTTTC
TATTCTTTCTTAATTACTCAACTCATTTTATATCTTCAATTTCACGATAATTAATTCTTCTACGAAAAAC
GTTTTCGTAAGCCTTACTTTATACCATACTAGTTTAACTCTTAGAATCCTATATTTCTTCTTAAATACCT
GTTTGCATTTAATTGGTTTTTCATCCATTCTTAGATCAAATCTCCATTGGTGAACATTCAAGAATCCAAA
TTCACCTTGCTCTCACACCAAAAAAAAAGAACATAGAGGAGAGAAAACCAAAAAGTGGTGAAAAAAGTGA
AGAAACACACCCATCAATTGTCATGAATTCTAATCAATCCCATAAATAGTCATGCGTTCATAAAAATATT
AATAATGAAAATAGTAATCATATCATCGTGCAATGCATTGAAAAAAATAGGTGAGAAAGCTATATTTAAA
ATTGAAAATGGAGTATACTTTTGATTAAATACTAAAAAACATTTTTAGGAACAATATAATGAATATTGTA
TTTAAAAAAACATTCCATTTGATTAAAAAAATTGATTACCATAATATATAAAATTAAAATTTATATAATA
CTTAATTGATTCATTTACTCAAAGTATATATTCGTTGTAATCATGATAATTAGTATAATTCGGTATTTTT
ATGAGTTAAAAAAAGAAAGCTGTAAAAATGATCAGTTATAAACGATATATAAGACATGAATATTTTGATA
AAAAAAATGATAAATTGTATGGCATAAACTTGATTATTTTGAGTGTTTTAAGATGTGAAATTTTTAGTTA
TACTTTGTCTTGTTTTTTTATATCTTTTAATTGAATTAAAAATTAAAAACTTTTCTCATACCGATAAGTC
ATACCAATTTAGGGCAAAAACTTTTCTCACAATTTTAATTATTATTCTTTTTTATATTTTTTAATTTTAT
TTTAATTAAAAGTGTTGTACGATGTACTTAACTTTTTTTTTATATAACCCCCTCATGTCAAGTTGGAGAA
TTGGATTATCCATCCAACTTGATACAGGCATACGTTCCAGACCTAAAATGAAATAATAATATTAAAAAAA
ACTTGATTCAGAATTATCGATCAATTTTCTTTCTGATATAACTAACTATATCTACAACAATTATGTTTTA
GTGATGTGTCTCAACTTGGCTGTTGCTTAAAATTTTCTGATTAATTATCTGTTTTATATTACTCATATTG
GTATATAAAAAGTGATTATCACCATTAAATTATTTTTTTTTTCTAGTGGATACAGTGCTATACCGTGTAT
CTGGTATTGCTTTAATTTTTATAGTCGTATATCTTGTATCGTTATATCTCATTATATTGCGCCGAGTAAT
TAATTAACTTAGCCAGAGAATATTTATATATTATAAATGAGATTCCTCGAATTTGATCAAAGCTTGATTA
GTCTTGTATGTCGGTATAAATAATTCAAGAAAACAAATATCAAGACAGGACAAAATCATAAAATAACAAT
ATTGTCACTCTTTTCGGATTTTTTTTAGTGATTGAACAAAAAAAATTCAAACAAAAACATTTCGTTCCTT
TTAAATTATGAACACTTTAAATTTGGAGTTTGGATAGTAAAATATTTTAAAACGAATTTTCACTCCGTAT
AATAAAGGACTCATTTTACAACATCAAACAAACAAATATTTAAATTGAATTTTTATTAGAGTTTAATACC
TATATATGTAATATCAAAGATGGTGAATTATAGTTGAATGATCATATAAATTTTTTTACATAATTAGTAT
ATAATTTTTTTTCTTTTTTATAAACTTATATTTTTTAATAAATTTTATATGTAATGAATTTTTATCAATT
TAATTATTAAATTGAAAATTTTCATGAAATTATAAACACACATTATATAGTAATTTGACACAAATGATTA
ATGTATTAAAGTTAATGAAACACATTACATACAGAGATAGGAGATAGGAAGGATTTAATTTGTATTATTT
TAATAATGTAAGTCAAAATTATTTTTACACTTTTAAATAACTTTTTACTAAACAATTTTATTATAAAAAA
TATTAGATTGAAAATTCCTATTATACAGATTATGTTTATAAAAAATTTATCACTTTAAACATGTATATGT
GCATGTTGGATATACATATAGAAGATGACTAAAGATAAGATGAGGTGCTCGTCAAAACTTCTACAAAAGA
ATTGGTCAAAATATTTTGAGTCAGTGAATATGCTAGTCACAACCCTCTTAACTTGATTTTAAAAATAAAA
TAAAATAAAAAACCTCTTAACTTGCTTCAAAATGAAACCTCTTGCATTAATCCAATCGTGCATTGAATGA
GTATAAAATAGTCTACAGTGGTTAGCAACAGTCTCAACTCTCAAAAACTTGAACCAAGTTGTATTAATTA
AAAAATATATACTGTATTCTATAACTGAAAATATCAATTGGCAATAATTTAGGAGCAGCCGCTCCCACAT
TCATTTACTAGACAGCTACTATTTTCCTTCCTCTATATTTGAATTTGAATTCTTTTAAAAAAATTGTTTT
TCTTCTTTATAAGACTCTCTTCAAATATTATTTCTTACGTTAATTTTCTTATCAAAATATTTTTAATTAT
TTTAAAATTTTTTAGTCAATAAATAATAATTATTATAAATTAATAAAAACAAATCTTTTTTCTCTCTTAT
AAGGATTGAGAAAGATGACCAGTATAAACTAATAACAGAAACTAAATAATTATTGTTCTTTCTTCATACA
TTAATTAGTTAAATGAACAATAATTAAATGAAAAAAATTGAGATGTTGAGTCTCAATAATTTTAAAAGTA
ATTTGGAAAAAATAATGTAAATTGTTAATAAACTTAATGTTATTAATTCAATTAATTAATTTTTTTATTC
TTGTTAATTGGTTAAAAGATTTTTGTGTATAAAGATGAAAGAAGTAGGTATTTTATCATCATCCAAGGTT
ATTTGATTATTTTTCACTTGTGTTTTATTTTAATTTAAAGGGTAGACGGACAATACGGGATCGATGAAGG
TTAATTATTGAGTTAAAAGGAAAAAGAAATTCAAGTTGGTGGAAGTTGGTGAGCTTTGGGGGGGAAAGTT
ACGAAAGGGACGAAGAATAAAGTTCATGAGAAGGAACGAATCATTAGAAAAGTTTCAAGAGTAAAATAAA
CGGTAAAACTAAAACCAGTAGCGAAGGAGATAAAAATCCATAAGCTAATAATATATGCCTAGTTGATAGA
TGAAATTAGGGAGAAATTCACAGGTTAGAAATAGGTCAGATGTCTTGTTAGTGGTTTGCATGTTTGGCTC
GCATTAAATTTAATAATATCTAAAAAACATTGATGATAATAATATCTAAATTTACACTAAATAAGCTAAG
TTAAAATTATTTTAAGGCTTATTTAATGATTATACTACAAAGGTTTTAAATCATTTAAGAAATCTTTGAC
TACAAAAAATTAGCTTATTTAAATATATAACATAATAAAATATAATATATATACATATATATATTTATTA
TGTTATATTATATTATATTTTTACTCTAATCTTAAAATTATATATATATGCATGCGCGTGCATGAGTTAA
TTGAAGTGGGATTAATATAACTTAATTAGTGACCTCGATTCTAGATCATAAATATGCAGGTATATTAAAT
ATTAGAATAAAAAAATTGTTGTTTATAATAATTGTATATATGTCGCTAGCAAGATTGCTTTTTTAAAAAA
AATGCATGTAATTTGCTATTTCAAAAATTTAAAAATGACATGTGATCAATATACATTATTTTTTAAAATA
AAAAAACTTCTTTTTATTAATGATTAAATTGTCTAAAATTATGATTATACATTTATTATTTGTATACTTT
TATTGACTATGTTTTATGGTTTTATGTGTTAAGCTTTGGTGTATATAATTAAAATGAGTTTAATATTTAT
GTATTAATAGTATAAAATTTATCATACATGATGAATGGTGAAATTTTGAATTATGATTAAATAATTATAT
AAAAAAATTTACATGATGAATGAATAACTTTTTTTTTCTCAATTAAAATTATGATCCTTTGTCGATATGT
TTTACTGTGTCGACCTTTTTTTTCGGGGGAGAGGGGACCAGTAGGAGAAGTAGTATTTAGTAAAAGAAGG
GAGAGAGAAGTTGACTTATCCTTTAATTAGTTTAGAGAAAATTAGACGAGAAGGAAAAAAAATAGGCGAA
AGTCACTTTTTCTTTCTATCTCTACCAAGAATGTTGATGAAAAAGTGGGGAGCAGAATTTTAAATTTTTA
TTTTCATATTTATCCTTCTCCACATTTTTGTTTTCTTCCATTTTTTTATAAAATGATTTATTTTAGGGCA
TAGTTAACTTTTCAATTTTTTTCATTTCTATTCGATCAAATAAATAGAAAAATAATTTTACTTTTCTTTC
TTTTAACCTTTTTCATATTTCTCTCATAACGAACAACTTATTAATTTACCTCTTTTCCCACCACTTTTTG
TCTATCCAAATTCTATCTTTGAATTTTCTTCCTTTTCATTTTGTTTCTCAAACCAAATAAAGAAGATCGA
GTTTGGATAAATCATAAAGTTATATACCTATAAATAGAAGAACATTAAATGATCAAAGGACATAAAATTA
ATTAATTAAATTTTGACATAATTTAAAATAAATTTATAAATCTCAATTTTTTTCTATAAATCATTTAACT
TTTTTATAAATACTTATAAACTTAATAAAAATTAATATTTTTGTATATATAAAATTCTTAACATTGTAAA
TTTATAATTAAAAAATCTATAAGTGAAAAGCTAAAAAAGAGTTGGGCCTAGCTAGGCATTATAATTAAGA
TAACGATTTAACTAATAATTCATTCGATAAGAGTTGCTTTTGTTATATATAGGTGCTTTTAAATAAGTTT
ACATTGATAGATTAAGGTAACAAAAATGACTTTTGGTATCGACTCATATAATTTATTTACTTTATTTTAA
TATCTTTTATATACAATTTATCAGAATAATTACACGGTTTTTAAAATGAAATAAGCTCAAATAAATTTTC
TAGAAGGCTTTTACAGACATCGATCCCCAAGTATGTGTTTGGCTTTACATTTGAAAAATTCCAAACTATG
ATTATTGGCAAATTTGTTTTTTGTACGAAACGTTTGTTTAAATAATGATCTGGAGATTACAATGAAACAC
TAAACATATTATAATTTGATAAATTATTAGGTGACGTAAGCAGAGTTAGATTTCAGTTCTGTATGCTCCT
CACATGCCTCTAATATCTCAATTGTTTCTTATATATAAATTGTAAGAGGCTGACACAGAAGATTTTCTGA
TCAGTCATCAAATAATTGAACTCTAAATATATTGCTCGTTATCATATATGTAAAATTTTATCTTGCCTAT
GCTTGTTAATTTTGTACTCTCGAACATGAATTTGGAAACTTAATTAGTTCATAAGATAATAATGCATATC
AACCCGAATCATTCACACATCAAAGCAATGTTCACTTCAATGGGAATATAAATTCTTTAAAATCATCCAC
TAGTAATACACCTAAATGCTACTAGTAATATAGTTGTGACACCATGCATGTTTGATTTTTAGCCCAATTT
CAATTTGTTGGCGTAGCTTTGAAAATTCCTAAACAGAACAGTAAGATGATCCATGGTGCATGGTACTGAG
ATAAGTAAAATAAATCTTTTTGAGAATTGATTTATCTTTTCAAAGGTTTAGAATTTTATTATGGGGCGAT
TAATTTCTAATTAGCACCTTTGACTGTCTCTTTTGCGTAGACAAATCTGCTATTACGTAATAGGTATATC
CATTTTATTCAATCGTTATTATATCAATAATATATATTATTATGTAGACATCAATGGATCGGAATATTTT
AAGAGGCATTCAATGGTCAATTTATGTTTTTAATTTGTTTCTTTTTTTTATACTAAATTAGGTTTCCTCC
CTAGCTAAGCATCTCTTTGAAAAATTCAAAAATAGATATATATTGAATTAAATTGATTAAAAGCTGAGTA
TTTCAGTTATTATTATGTATGATTTATCACTTTTCTATCTACCCAAAAGGTTTATTAGTTTATGGTTTCT
GCAATAAAACATATTTTAATTTGTTACCTTTCAGTCTAACATATTCTATAATGGGTTTCGCCATCACACG
TGAACTTGCTTCTTACTTCAGAATTTTGCTATGTCTGTGAAGGATCC

| | | | | |
|---|---|---|---|---|
| SEQ | NO | 5> Intergenic regions Coordinates in SEQ 4 | 7947 bp | 10862 bp |
| SEQ | NO | 6> Intergenic regions Coordinates in SEQ 4 | 15479 bp | 18721 bp |
| SEQ | NO | 7> Intergenic regions Coordinates in SEQ 4 | 30207 bp | 36448 bp |
| SEQ | NO | 8> Intergenic regions Coordinates in SEQ 4 | 39204 bp | 48767 bp |
| SEQ | NO | 9> Intergenic regions Coordinates in SEQ 4 | 52465 bp | 54579 bp |
| SEQ | NO | 10> Intergenic regions Coordinates in SEQ 4 | 57894 bp | 59654 bp |
| SEQ | NO | 11> Intergenic regions Coordinates in SEQ 4 | 64305 bp | 64551 bp |

SEQ NO. 12 >gene_1|GeneMark.hmm|271_aa
MSTSSSSQSLKIGIVGFGNFGQFLAKTMIKQGHTLTATSRSDYSELCLQMGIHFFRDVSA
FLTADIDVIVLCTSILSLSEVVGSMPLTSLKRPTLFVDVLSVKEHPRELLLRELPEDSDI
LCTHPMFGPQTAKNGWTDHTFMYDKVRIRDQATCSNFIQIFATEGCKMVQMSCEEHDRAA
AKSQFITHTIGRTLGEMDIQSTPIDTKGFETLVKLKETMMRNSFDLYSGLFVYNRFARQE
LENLEHALHKVKETLMIQRTNGEQGHKRTES
SEQ NO. 13 >gene_2|GeneMark.hmm|673_aa
MKPHTPASSFVTRLPHVPYFRGATAARAAPPDPPHDAPGGLEFRRVSTAKRRRVSLSVCH
ASRVTAASNPGGSDGDGDTRARSSRRGVLMAPFLVAGASILLSAATARAEEKAAESPLAS
APKPEEPPKKKEEEEVITSRIYDATVIGEPLAIGKEKGKVWEKLMNARVVYLGEAEQVPV
RDDRELELEIVKNLHRRCLEKEKLLSLALEVFPANLQEPLNQYMDKKIDGDTLKSYTLHW
PPQRWQEYEPILSYCRENGIHLVACGTPLKILRTVQAEGIRGLTKDERKLYAPPAGSGFI
SGFTSISRRSSVDSTQNLSIPFGPSSYLSAQARVVDEYSMSQIILQNVLDGGVTGMLIVV
TGASHVTYGSRGTGVPARISGKIQKKNHAVILLDPERQFIRREGEVPVADFLWYSAARPC
SRNCFDRAEIARVMNAAGRRRDALPQDLQKGIDLGLVSPEVLQNFFDLEQYPLISELTHR
FQGFRERLLADPKFLHRLAIEEAISITTTLLAQYEKRKENFFQEIDYVITDTVRGSVVDF
FTVWLPAPTLSFLSYADEMKAPDNIGSLMGLLGSIPDNAFQKNPAGINWNLNHRIASVVF
GGLKLASVGFISSIGAVASSNSLYAIRKVLNPAVVTEQRIMRSPILKTAFIYACFLGISA
NLRYQAVFEVDGG
SEQ NO. 15 >gene_3|GeneMark.hmm|278_aa
MGTMSHVRACLEKQAVLPIHNARWNSKRRLFIQHLAYGQKHINSHMKGKSTLVSSAKTAE
AINTSNSDASSDNTPQGSLEKKPLQTATFPNGFEALVLEVCDETEIAELKVKVGDFEMHI
KRNIGATKVPLSNISPTTPPPIPSKPMDESAPNSLPPSPPKSSPEKNNPFANVSKEKSPK
LAALEASGTNTYVLVTSPTVGLFRRGRTVKGKKQPPICKEGDVIKEGQVIGYLDQFGTGL
PIRSDVAGEVLKLLVEDGEPVGYGDRLIAVLPSFHDIK
SEQ NO. 16 >gene_4|GeneMark.hmm|298_aa
MANNFLDVFCWIQNLPPISEWETSSMSLNICSSSSSCQPRLNLTAILLYGSNKNS
TTFIR
FPNLDSTASDNLSDVFNLSDSRQASHMIMKLLGSNLEELWMRSLNLAVQLYCHLM
VMDVE
NSKSSPASERLQFSLRYHHVEGVLQFNHKVLIKDEWAEIMVDIDNVRCDVIELVN
EFLMK
QRGAGAAEKHFPSRISLQLTPTIQDQVLSLSVGKSSENPRKEIGVDKSVEASFEA
SNPLA
LKVSAGESPQPLVYGYSANLNWFLHDCVDGKEVLSSKPSKFAMLNPKSWFKNRY
SSAY
SEQ NO. 17 >gene_6_7|GeneMark.hmm|602_aa
MEPAKTIHNNVKYSPIFLAIFVLILASALSSANAKIHEHEFVVEATPVKRLCKTHNSITV
NGQYPGPTLEINNGDTLVVKVTNKARYNVTIHWYNIKLASMAFFSGHGVRQMRTGWADGPEFVTQCPIRPGGSYTYRF
TVQGQE GTLWWHAHSSWLRATV
YGALIIRPREGEPYPFPKPKHETPILLGEWWDANPIDVVRQATRTGGAPNVSDAY
TINGQ
PGDLYKCSSKDTTIVPIHAGETNLLRVINAALNQPLFFTVANHKLTVVGADASYL
KPFTT
KVLILGPGQTTDVLITGDQPPSRYYMAARAYQSAQNAAFDNTTTTAILEYKSPNH
HNKHS
HHRAKGVKNKTKPIMPPLPAYNDTNAVTSFSKSFRSPRKVEVPTEIDQSLFFTVG
LGIKK
CPKNFGPKRCQGPINGTRFTASMNNVSFVLPNNVSILQAHHLGIPGVFTTDFPGK
PPVKF
DYTGNVSRSLWQPVPGTKAHKLKFGSRVQIVLQDTSIVTPENHPIHLHGYDFYIV
AEGFG
NFDPKKDTAKFNLVDPPLRNTVAVPVNGWAVIRFVADNPGAWLLHCHLDVHIGWG
LATVL LVENGVGKLQSIEPPPVDLPLC
SEQ NO. 18 >gene_8|GeneMark.hmm|763_aa
MSESSITTVFNDARTGQMIVCLKNDRTVGSLGVWMGDNPFDFVVPVTLFQIILVS
LLSKA
LHYVLRPINTPKFICCVIAGILLGPTFLGRHEEILGALFPVRQSLFLNTLSKIGT
TYCVF
LTCLKMDVVTTLKSAKRCWRFGVFPFLASFLVTVTLFSLYSPNGNANQNQMSIYH
FPNIF
TLSSFAVVSETLMELNLVATELGQIALSSAMISEILQWTTMELLFNSKFSMRFLI
VLLIG
ATGFAVLLLLIIRPLVNIVLERTPPGKPIKEAYVVLLLLGPLVMAAISDTFGIYF
VMGPF
LYGLVLPNGPPLATTIIERSELIVYEFFMPFFFLLIGTRTDLTLIHEHWEVVLVV
LAILF
VGCLVKVIDTEVFSVAVMSVVVMTSICIPLIKSLYRHRRVCKTQTIQEGSVKTIQ
NITEN
TPFNIVSCVHTDEHVHNMIALIEACNPTTQSPLYVYVVHLIELVGKSTPILLPMN
KNKRK
SLSVNYPNTNHILRAFENYSNNSSGPVTVLSYVNVAPYRSMHEAVCNLAEDNSVH
LLIIP
FHQNDQTLGSHLASTIRNLNTNFLANAKGTLGILVDRYSVLSGSSSKLSFDVGIF
FIGGK
DDREALALGIRMLERPNTRVTLFRFVLPTNEDSRFNGLVENEDENLESTLDESLI
DEFIA
KNDISSDSVNVVYHEAVVEDCIQVLKAIRGMEKDYDLVMVGKRHSMGNFVEEEMS
NFMDN ADQLGILGDMLASNEFCNGKVPVLVMQCGDEKRVKQLEKVCHI
SEQ NO. 19 gene_9|GeneMark.hmm|871_aa
MATSRGNGIVSSYWDSHGQWQVCVEDDRNVGSLGIFIGDRPFEFVLPASKNTQIH
LQRPV
SPLENSRKRHKLDRCTSPTRQCDNVVYSSKLKVVEAVPNYSSKSPMLDSTSPIVV
EECLR
YGGGIILGPTFLGRNKTYWQVLFPPRQTEYLVMASLTGAVYFVFLVALKMDVLMT
IRAAK
STWRLGVIPFLASFVVILALLCLYYHPQQISSASLTIARVSVSCLMSLSNFPVVS
DAMLE
LNLTATELGQIALSSSMINDIILWLFIVMHSFTSNVDVKKSIALLGNWCLLVFFN
FFVLR
PTMKLIAMRTPVGKPVKELYVVLILLGVLVMAGVGDLMGVTFLMGPLIFGLVVPS
GPPLG
TTLAEKSEVLTTEFLLPFFFVYIGINTDLSALEDWRLFLTLQGVFFAGDLAKLLA
CVLVS
LAYNIRPKHGTLLGLMLNIKGITQLISLARFKKQKMLDEDTFSQLVFCVVLITAI
VTPLV
NILYKHRPRVHAESLFEGELRTIQSTPRNREFHIVCCVHNEANVRGITALLEECN
PVQES
PICVYAVHLIELVGKSAPILLPIKHRHGRRKFLSVNYPNTNHIMQAFENYSNNSS
GPVKV
LPYINVAPYKSMHDAIFNLAQDNMVPFIIIPFHENGNIDLVGHVAASIRKMNTRF
QAHAP
CTLGILVDRHSRLGASNNNNMYFNVGVFFIGGAHDREALALGIRMSERADTRVSL
FRFVI
VNKKPCGCKIILTREEREEEEEDTMLDEGLIDEFKSMKYGIGNVCWYEITVDDGV
EVLEA
VHSLEGNYDLVMVGRRHNDGSLNGKEMTTFMENADALGILGDMLSSVEFCMGMVP
VLVTQ CGGVKISSSSNNKLDRVGSVNVSQKRLSVHK
SEQ NO. 20 >gene_10|GeneMark.hmm|1245_aa
MEASVLLGNSYGVVARVAVAGLRGGVMAFEAYLSRSWRAVELIKFESGTTTLYFV
DNHHM
TIKKGSFSDVRVRSRKATLSDCSFLRTGIDICVLSASQGNDNSDESSANHVWLDA
KINSI
QRKPHNPECSCQYYVNFYVNQGSLGTELRTLRKEVKVVGINEIAILQKLERNTCQ
HKYYR
WESSEDCSKVPHTKLLGKFISDLSWLVVASAIRKVSFCARSVENNIVYQILGSDA
TTSSL
YMDSEISVVNFKVNEDGMQMPVIHLVDLFETDTNTSGDKHDSHYDEVPSSYGFEG
LRRSK
RRNIQPERYSDCGNVSEIKVGNVRTWPYKLNKRKDDDGGGEESLPLAQENSDNSQ
KVNEL
SSCREIIVYHGRNETLELKSGEANQTQLASVPLLQEGDSLALEHHHLNDNVTRRS
DAYYS
TPKLKRKRLVDLEADVDFDPGREGINSNKGVSEKRHGSSWYSRSRSHAAEHSYKD
RSLNA
TAYKEMIDSYLKDVNRTPTTEEPPVMDQRKEIGNFGQKKEAEIPEREDEEQISE
IDMLWR
EMEMALASSYLEETEGSNSANFAKTTEESNRTCPHDYRLSEEIGIYCYKCGFVKTEIKYI
TPPFIEMQRSVRHQEEKQCNGKDTKEKASKDDDFHLLSTHAPTDEHNSMEHDNVWKLIPQ
FREKLHDHQKKAFEFLWQNIGGSMEPKLMDAESKRRGGCVISHAPGAGKTFLIIAFLVSY
LKLFPGKKPLILAPKGTLYTWCKEFNKWEISMPVYLIHGRGGTQKDTEQNSIVLPGFPNP
NKYVKHVLDCLQKIKLWQEKPSVLVMSYTAFLALMREGSEFAHRKYMAKALREGPGILIL
DEGHNPRSTKSRLRKGLMKLKTDLRILLSGTLFQNNFCEYFNTLCLARPKFISEVLDTLD
PITRRKSKTVEKAGHLLESRARKLFLDKIAKKIDSGIGNERMQGLNMLRETTNGFVDVYE
SENFDSAPGLQIYTLLMNTTDKQREILPKLHTRVDECNGYPLELELLVTLGSIHPWLVKT
TSCANKFFTADQLKQLDKYKYDMKAGSKVKFVLSLVFRVMQREKVLIFCHNLAPVKLLIE
LFEMFFKWKKDREILLLSGELDLFERGKVIDKFEEHGGASKVLLASITACAEGISLTAAS
RVIFLDSEWNPSKTKQAIARAFRPGQEKMVYVYQLLVTGTLEEDKYKRTTWKEWVSSMIF
SEAFEENLSHSRAVNIEDDILREMVEEDKSKTIHMILKNEKASTN
SEQ21 >LRRpeptideforantibody
CTLSRLKTLDISNNALNG NLPATLSNLS
SEQ NO 22 >gi|17981611|gb|AAL51087.1|AF456323_1 cyclophilin [Glycine max]
MPNPKVFFDMTIGGQSAGRIVMELYADVTPRTAENFRALCTGEKGVGRSGKPLHYKGSSFHRVIPSFMCQ
GGDFTAGNGTGGESIYGAKFADENFVKKHTGPGILSMANAGPGTNGSQFFICTEKTEWLDGKHVVFGQVI
EGLNVVKDIEKVGSGSGRTSKPVVIANCGQPS
SEQ NO. 23 >gi|33325957|gb|AAQ08403.1|methionine synthase [Glycine max]
MASHIVGYPRMGPKRELKFALESFWDGKSSAEDLQKVSSDLRASIWKQMADAGIKYIPSNTFSHYDQVLD
ATATLGAVPPRYGWTGGEIGFDTYFSMARGNATVPAMEMTKWFDTNYHFIVPELGPDVNFTYASHKAVDE
YKEAKALGVDTVPVLVGPVTYLLLSKPAKGVEKSFSLLSLLPKVLAVYKEVIADLKAAGASWIQFDEPTL
VLDLESHKLQAFTDAYAELAPALSGLNVLVETYFADIPAEAYKTLTSLNGVTAYGFDLVRGTNTLDLIKG
GFPSGKYLFAGVVDGRNIWANDLAASLTTLQGLEGIVGKDKLVVSTSSSLLHTAVDLVNETKLDDEIKSW
LAFAAQKIVEVNALAKALSGHKDEAFFSGNAAALASRKSSPRVTNEAVQKAAAALKGSDHRRATNVSARL
DSQQKKLNLPILPTTTIGSFPQTVELRRVRREFKANKISEEEYVKSIKEEIRKVVELQEELDIDVLVHGE
PERNDMVEYFGEQLSGFAFTVNGWVQSYGSRCVKPPIIYGDVSRPKPMTVFWSSLAQSFTKRPMKGMLTG
PVTILNWSFVRNDQPRSETTYQIALSIKDEVEDLEKAGITVIQIDEAALREGLPLRKSEQAHYLDWAVHA
FRITNVGVQDTTQIHTHMCYSNFNDIIHSIIDMDADVITIENSRSDEKLLSVFREGVKYGAGIGPGVYDI
HSPRIPPTEEIADRINKMLAVLEKNILWVNPDCGLKTRKYTEVKPALTNMVAAAKLIRNELAK
SEQ NO. 24 CLE2, GmNIC1, LjCLE-R2 and LjCLE-R1
RLAPGGPDPQHN
SEQ NO. 25 CLE2, GmNIC1, LjCLE-R2 and LjCLE-R1
DLPLAPADRLAPGGPDPQHNVRAPPRKP
SEQ NO. 26 CLE 30, GmCLE30, GmRIC1
RLAPEGPDPHHN
SEQ NO. 27 T, GmTDIF, ZeTDIF
AHEVPSGPNPISNR
SEQ NO. 28 CLE 36, GmCLE34, AtCLE36, MtCLE36
SKRRVPNGPDPIHNR
SEQ NO. 29 CLE 36L, GmCLE34, AtCLE36, MtCLE36
RAELDFNYMSKRRVPNGPDPIHNRRAGNSGR
SEQ NO. 30 CLE 3, GmCLE3, AtCLE3, AtClv3 unmodified
RTVPSGPDPLHH
SEQ NO. 31 CLE 3L, GmCLE3, AtCLE3, AtClv3 unmodified
KGLGLHEELRTVPSGPDPLHHHVNPPRQPR
SEQ NO. 32 CLE N, HgCLV3
KRLSPSGPDPHHH
SEQ 33 Complete sequence of BAC H28F23from LgB1 (Chromosome 11)
that conatained a syntenic homeolog of Rfs2/ Rhg1 and linked genes
>soybean_14G5
CGGCTGGTTGCGCCAAGATCCGCTTGCGGAGCGGTCGAACATCCATGCTGGGACTTCAAG
AAGTCGAGCAGAAGAAGAACCAGAAAGGCTGCACCGGAAAATATGCGTCCCTTTGGAGAG
CGCCTCATGGACGTGAACAAATCGCCCGGACCAGGGATGCCACGGATACAAAAGCTCGCG
AAGCTCGGTCCCGTGGGTGTTCTGTCGTCTCGTTGTACAACGAAATCCATTCCCATTCCG
CGCTCAAGATGGCTTCCCCTCGGCAGTTCATCAGGGCTAAATCAATCTAGCCGACTTGTC
CGGTGAAATGGGCTGCACTCCAACAGAAACAATCAAACAAACATACACAGCGACTTATTC
ACACGCAAATTACAACGGTATATATCCTGCCAGTCAGCATCATCACACCAAAAGTTAGGC
CCGAATAGTTTGAAATTAGAAAGCTCGCAATTGAGGTCTACAGGCCAAATTCGCTCTTAG
CCGTACAATATTACTCACCGGTGCGATGCCCCCCATCGTAGGTGAAGGTGGAAATTAATG
ATCCATCTTGAGACCACAGGCCCACAACAGCTACCAGTTTCCTCAAGGGTCCACCAAAAA
CGTAAGCGCTTACGTACATGGTCGATAAGAAAAGGCAATTTGTAGATGTTTGGCAAGTGT
AGCGGTCACGCTGCGCGTAACCACCACACCCGCCGCGCTTAATGCGCCGCTACAGGGCGC
GTCCCATTCGCCATTCAGGCTGCGCAACTGTTGGGAAGGGCGATCGGTGCGGGCCTCTTC
GCTATTACGCCAGCTGGCGAAAGGGGGATGTGCTGCAAGGCGATTAAGTTGGGTAACGCC
AGGGTTTTCCCAGTCACGACGTTGTAAAACGACGGCCAGTGAATTGTAATACGACTCACT
ATAGGGCGAATTGGAGCTCCACCGCGGTGGCGGCCGCTCTAGAACTAGTGGATCCCCCGG
GCTGCAGGAATTCGATATCAAGCTTGTTGGTTGCAGAAAATCATTTCGATGAGTTTTTTT
AACGGACAACTTTATAATATAATTATTTTGAAGAAAAAGTGAGTTTTTCTTTCTAATTTA
TGAAAAAAATATCAAATTATATATTTTTTAAATTTGTTTTGCTAATTTTCGCTAAAACTT
GCAATTTTTAAACTATATTTTTTCTTAAAAGTCTTCTGTCTTTTTTTTTATTTTTATTTG
GTGGGGAAGACACTTTTTGTCTTTAAAAGAGAATAAAAAGTAGAAAGGCCATCAAATAGA
CGCTGTACCAAAAGGCAAACCTATTGTTGACTTGTTTCATAAATAATTGACATCTTTAAT
AATTTCATATCATATGTCTTTTTCCTAAAAACGCTTCTCTTTTTCATGACTACATATCTT
GTTTTAATTATGTCAAATCTTTTGCAAAGTAAATTTATTAAAACAAGACATGTGACATGT
TCATTGTAGTCAAAATTAAGAAGAAATCAAACCTTTGCTAGAGCACTTGTAAAGATCACC
AGGTTGACCATTGATAGTGTATGCATCAGACACGTTTGGAGCTCCCCCAGTTCGCGTGGC
CTGCCTCACAACATCAATAGGGTTTGCGTCCCACCATTCCCCTAAATATTCACCATCATC
ACTTTCACCGTTTAATCTTTGTCCAAAAGCAAATTAAACAACAAAAAACAATAATTTCTC
TCTCTCTCTCTCTCTCTCTCTCTCTCTCTCTCTCTATATATATATATATATATATATATA
TATATATATATATATATATAGTCTCTTTGCTTCAAAAGTAGTATTAATTAATTCCAACTA
TCACAATCACTTTTTAGTTTTTAATTAATCTTAATTCTTTTTTAGATTGTTGCCTCCATG
TTTTTCATCAATGTTATAATTACAAATAGTGCATGCATGGGTAAATTAATTAACAAGGAA
AAATTACCAAGAAGAATGGGTGTTTCGTGCTTAGGCTTGGGGAAAGGGTAGGGTTCTCCT
TCCCTAGGACGAATGATTAAAGCACCGTAAACAGTGGCCCTTAACCATGAGCTATGAGCG
TGCCACCAAAGTGTGCCTTCTTGTCCTTGAACGGTAAAACGGTAGGTGTAACTTCCTCCA
GGACGAATCGGGCACTGAGTCACAAATTCTGGTCCATCTGCCCATCCTGTTCTCATTTGC
CTAACACCGTGCCTAAGAACATATATTATAATTAAGAAAGAATACGAGCAATAAAAATCA
CTTTCAATTATATTAATTCACTCCTAAAATGAAGTCTTAATTTGGACTCTTTCTTGCTTT
TAAAACTTTCTTGATGGTATCACATACGTACGTACGTACGTATCATGTGGTATGTATTAG
GATTATGAGAAATTATTAGTTTTACATATGTGGTTGTCAATGCTTTTCATTTTCACATTC
TAATGGAACGCATTCACCCTTTGGGAGATAGAAAAATGAGAATAGAAGAGAGAATACATG
ATAAATATCATGAGAAGGAAAGTAAGGTAAAAAATTAGAATGCCAAAAAGTAAAAAGTGT
ATATATATTTTTATATTATTCTTCACGTGGCGTAGAGAGAGACTCTTTCCATTTTAATAT
AAAAAGGAGTGTAAGGAGTAGTATATGTATATATAACGTACGTGAGAAAAATGTTTTTCG
TAAGAGTGTAATGTCTATGTCTATAAATCGTGGTAAGGACTAAGGAGACGTGCAAAATTA
AGTAAAAGTGTCTTCATAATGTAAAATGAAATTAAGATGCTAGCTTGATATTATACCAAT
GAATGGTCACATTGTAACGAGCTTTGTTAGTGACTTTGACAACCAAAGTGTCTCCATTGT
TGATTTCCAACGTTGGGCCCGGGTATTGCCCATTCACGGTGATGCTGTTGTGGGTTTTGC
ACAGCCTCTTCACTGGAGTTGCTTCAACCTATTCCATTACAATCAGCTAGGTAATTATTT
TCTAACGAAAAAGAGTAAAAAATTAAACAAGAAAAAAATAAGCCCCGAGAAAATAATTGA
GAAAGAGAAAGAGGGTGAGAGAACGTACAACAAACTCGTGCTCGTGAATTTTGGCATTTG
CTGAAGCTAAGATCAGAACAAAGATCATGGCTAATAAGAAGATGGAGGAGTGTTTGGCAT
TCATGTTAATGAACTTGACAGACTCCATTTTTTTATGAGCAATTAACACCTTAGTGTGAA
TGCTGAAGGTTAAGGTTGCAATGCATGTAAGAGTGGGAGAGGGTGCTTTTATAGGGTGGA
TTAGAGGTGAGGGAGAGAGATAAAGTCCTTTGAGGTTGGCAGATTAGGTTAATTTAAGGT
CAAACTCAAAAGGGAAACTTAACTTGGTTCAAGCATATAAGAGTTGTTGCACCCGCTAGT
TAATTAAGTGCATGAATGCTTACTTCAGAAGTCTTTAGTTAGGGGTTGAAATTGAATTTG
TCCCAAGAATAATTATCTTGAGACTTTGTTTAGTTTATATGACGGGTTTTAATTAATATG
CTGGTTTTACACTTTCTAGCTCTCATTCAAGAGGCACGTAACAAGGTTAATTAATCCCTT
ATTTTACCAAGACCATTATATATATTAATCCCTTATATGTTCCTGATTCATTTTTATTTG
ATATATAACTGATATTTTTAATTTATACTAAAAACTAGTTAATAAAACTAGATTATATAT
ATCATGTGGAGATTGACTGTGAATAATAATTTTAGATTATGTGATAATTTCTCTCCTTTA
CCACTTATATATATCCATTCCTGTTTTAACAAAAGTATAACGTACAATCCAAAAGCAAAA
AGACTAATTAAAACAGAAAGCATTTTAACTTTATTCTTTCCTTAGCAAGTCAAGCTCGCC
ATTGCAAATTACCAACAGCCTTAAATTCTTGAAAATTAATTACGTGCAGAAAACTGCATA
TAATAATTTAGTGTAAACCGTCAACTTAACATTCTTTTCATGTAAGGAATTCTTGTGATT
TGTGGAAAAGGGTATATTTTGATGCTAAAGAGTTCAAGCCAAAGATCGTGCCACTTTTGC
ATTAGTCCAATTTGACTTCTTTTTTTTTCTTTTTCTTATTTAGGTGATTGATTGTGGGAC
AAAGCGCCCAATAATATTTATTCTCTTTGGTCATTAAAAAAAAGCTTTAAATCCTAACCT
CTAATGATAATTTTTTTAATGAAAAAGTAATCCGCGTTATTCAATTATTCTTTATACCCG
GATAATTAATTAGGCTAATAGTTTTGATTGATTATACCTAGATAAGTACCGTAGACTTCC
ATGATGCGTGCAGCAAATAAAATAAAGTACTATAATTAACTAAAGTGATTTGATTGCATC
TAACAAGAATTTAGTTAGAGATACTTATGAAAAATATTTCTCAAGTATTAAATTAGGTGG
TCTGTCATCCAACATAACTTAACTAATTTTATTTTACGCATGGATGAGATCAGCAAAGAA
TTACACGCACACGAATGTCATACTCAAAGATACGTACTTGGATGTACCAGGCACACATCA
TTTTTATTTTTGACTAACATTAGGTCACCATACTGCCACGTGTTCAGCTATAATACCATT
ACCATGGCTTAAAATTTTTCAAATGTTAGACAATATATAGGAGAATATGGTTTTTGCTAT
ATTTTCAATGATCTGTTAATTGTTTGATTCCCCTCAATTGTTTTTTTTAACACAGCTCGA
AAGCTAAGTAATGATCAATTACTTAATTGAAAGGACCTCTTTATGTTACATTAAGAATTA
ATTTTTCATTTGGTAAAAAAATGTTTTTTCATACCATACATATATAGGATACGATCAAAT
CATATTAGATGTGAAACTTTCACACTTTCATTACTTAAGAATATAAATTAATTCATATAA
TAATCATTGAGTTGAAAGTTTTTACAAAATAAAAAATTTATGAAAATTCATACAAATTAA
AAAGTAATAATTATTTATTATATTATTTTTATTTTGATAGATAATCTTATTTAGAATATA
AATAAATGCTTGTTGATGGCTCATAATATAACAAATGTTCAATTTATATAAAAAAAATAA
CATGCATAATTAAAAAAGATAATATCAAATTACTTGAGACTTTCACTGGGGTAGGTTGAT
GAATTGTTTTTATTCGTAGGAATCAGAAAAATGATTTATAATAATTCACTTAATATGTTG
AATCAATTCTACTAGATCCTTCTTTATAAGTTGCGATTTAACATAGGTAAACTAACTGCA
ATTTAATTTTATTTGCTTGGTCTATTTTGGGAGGATGAGTTGTATTTTATTTTGATATAT
GGTGGGTGGAATTTATTAAGAGAGGGACTCTTATAGTTACTCTTGGAATAACTTGATCAA
AATAAATAGTTAACATATATATTTAAATGATTGTATTAACGTGATGATTATTATGATTTG
GTAGAAAAAATATCAGAAAATGATAATGATTTCTCATAATATAATGTATTTATATATTTT
AAAGTTAGGTAGATAAAATTGAATTTAAATTGTTAGATATAATTAAAATACATTAACATG
ACTTTTAACAAATTGATATATAAACACTTAAAAAATTTTCAATTAATGACTATTTACATA
TATTGTTATTATGAAAATAACACCAATTACTAATTAGAAATCATCATTGACATTGCTTTT
AAAATAATTTTATAAAAATCGATAAATTTATTATAATATAAAAATTGTTTAATAACGTAA
GTGTACATCTTATTTTCTAAAATATAAATAAAAATTACTAGACCAAGCAATATGGACCTT
GTGCTTTCTGAACATATATAACACGAAAATTGCATAATTATTTTTCCACCTTCAAGCTAA
ATCCGCAACGTACTGTGGACCCTCATGAGTGTGAGGATCTTCCACAGGTCACTCACTTGC
TTTGACATCTGAAAGATCCTTCTCGAGTAAAGCGTGGAAACAAGAACAAGCTTGGACTGG
TCTACGATTTAGTGTTCTTATTCCAAGTAATATAAAAATCTTAAATCTCAAGGAATGATG
CAATGCTTCCTCTAATTGTCAGATATCCCTCCCACGCACCTAATCTGAATGTTGTGTCTA
AATTGGCATAAAAGGTTTGATTGATGAATGATGTCTACATGTGAGTTTGATAACAACAGC
TACCCCTTAGCCAAGCCACTAACTAGGACATTAGTTTTGGTTGGTTGTCAGACAAACCGT
TAGACCCTGAGAACGAAAGCGTGTCAAACAAAAGATAATAGACTTCAATTAATATAAAAA
GAGATGATCAGAAACCAAATTGAGATTTGATAGGTGAACTATAAATCATGACAGTGCATT
AGACAAGTTGGTAGAGTTTGTTACTGACTCATCTGATTCTTAAGAAAGGCAAAAATAGGA
ACTACACCAGATGTCGCTAGCTAGCGATCGATAACGTGCAAATTATAAATAAATGGCTTT
ATTTGAAAGTTCATCTTCATATGGTTAGTTATAATTGGGTCAAATTCTTAATTTATTAGA
AACGTGCACTTCATTTTGAGTGTATAAAGTTGGAAGAAGAAAAGGAATATAGAAATTAAA
AAAATAGAGAAAAATCAATAGATGTAATAATTAAGTTATGTTTATGTGATGGGAAAAGAA
GACAAAATAAAAATGAGAAGAAATTCTTATGAGATTGAAAAGATATGAAGTATATTTTTT
TAAGATGATATTTGTATACAATAATTTTTGTCGAAGAGAAGATTAATCTTTAGTAACTAA
CTAAAAATACACATAAGATAATTATTTTTCTTGTAACATAATTTATTTTATAAAAAATAT
TAATCAGAATTTAAATATGGATCATTTGATTAAAAAACATAAATTGTTAACTAGTGTATA
TTTATATACAAAAATCCAATTAACTATCTAACTTTTGATTCCACCACACATCAATATATA
TTCTCTCTGACCGAAAATTATGATGGTTAGTAAACGAAATTAAAGACAAATACACCATGT
TCTTTTGTTTCAAAATGGGGCCTTCTTAAGGGTCACGCCCAATGATACGTTGTTGTTTAA
GTTGTTAAAATGCATGTTACCCTCCTCATGAAGCTATAATAGGTTTCAGAGATAGTCTTT
AGAAGTTCGATAGAACATGTGGATCTGGGAGTGAGAGTGAGGTCGTATGTCGCTATAATG
CTGTATAAACTTTTGGTGAGCATGCATTGCCATTTTGTGGGTTTCGGTGATAGTCTTCTC
AAGTTCATTAGTCTTTACAAGTTCAATATAGATAGATAGAATTTGTGGACCTTGGAGTGA
GAGAGGTCGAAAGTCACCATAATGCTGTATAAACTTCTGATGGGCATCCAATGTCCCCTA
GGGGCAACAGGGTACCTAATTAATTGGTACCACAACGGGGAGAAAATCGATCAACACGTT
TGTGGAATATACATATATACCTAGAATTGAAGGGCAAGCTCAATTAATCAAGTTAAACAT
GAATTCAACTAATAGAAATTAAATTGAAATTTTGTTACTCGGGACCATTAGTTATAATAA
AATGCGTTGCACAACTTCAGTTATTTTGGATTTTTTGTTGAGTAACTCAATCAATTTTAA
ATGTTGCTTTCTAAATCAACTATTAAAAAAACTAATGCAGAAATACATCTACATCGTGGC
TAGAATCGATCTAGAAAACTTTTTTTAATGTAATTAATTTCTAACTTTTTGCATCAATAT
TATGAATAGAGTCAATCTAAAAAAAACTATATATATTGGTTCTAGTCTTTTTGGCATAGC
TACAACTGATTTAAAAAGTGACTTTCATTGATTACGATTATACAAATGTGGAAAGACTAT
TTAAAATTTATATATATGGAAGAGAATTCAGATATTTTAAGAGTAAGTATAAAAAATTAC
ATTAAAAAAAACTTCATTGATATATTAAAAAAAATCATAATATTATAAAAACCGAATAAT
TAAAGACGTGCACATTAGTAATGTACTATGGTAGAACATCCAATGCACATAATTGTTTGT
TGGTCAAATGATATTGGCAGTACTCTTTGATCTACCCATGAGATGGTGTCTCTTCAGCAC
CAACTTAGAAGTGTTAGTCTGAAGATTGTTTGTATATGTGGTTTTGGTTGATACTAGGTT
GTTGGCAGTGGTTGAAAGACATTTCCTCTTGGATTATCACACAATCATTTGTGGGATTTT
TTATGTTTTGCTTAAATATTCTTCATTTAAAGTTTCATTACTAATTATCAGTTTAAGAAA
TAAATAGAACAAGTAGTATTGATATTAAATTTTGCAAATTATATCCAAACCTAGTCGCTA
ACAATGTCTGAAATATTGTTACACTTGATTGTGTGATACTGTGATCTATTTATGTTTTCT
TTTTTGACTGATAAGTTTGATTTAACGATAGGATTATTTCCATCCTCTTTTTAGGCTGAC
ATTATATTTGGATAAAAGTTAGAAAAGTATTGTTGTGATTTGTGAATTTCAATATGCATA
TTTCTAAATTTCGATGTACAAAATGAGAAAGAAAGAAATTGTTGTTTTAGAAGTAAAACT
CTCTCAACTGAAGTACAAACATGAACTGAATCTCTTGTGAGGTTTCCACATGACATGGGG
GAAACAATGGGTGAATAAAGATTGGGTTTTGTAAGACTTAATTTGAGAATTGAGACAGAA
GAAGCCTTATTTGTTTGTGGGAGAATACGAGTAACTGACATGAGAAATGTGGCAATCTGC
AACTATTAAACCAAAGAAGATTATAAAAAGATATATATCAAAACCTCAAACATTTGGATA
ACATTCTTAAGCTTGTAATATTAATGTGCATAAGAGACTTTCACAATGACAGATCTGCTT
AAGCTTGTAACATTTGGATAAACATTCTTTTTTGTTTCTTTTTGTCATTGGTTATGAATT
AGTGTATGCTTTCTTTGTTTATGGGTTTTGTTTCCAAGTAAATTTGAATGTTAAACTTTT
TATTGAGTAAATTTTGAAGTTGGAATTATGTCGGGTGGTTTTTTCTTTCTTCTTTTTTTG
CCATAATAGGTTCTAACTTCCTTAACTATCCGTTATTTATCCCTTTTTTCTGGGTCTCTT
ATTTATTCCAGTTAGAGATATTTAATTCAAGATGTGATTCATATTGTTCACATCAGTTCA
ATTTTATATTTCTATCTCCAATAGATAAATTATATAAAATTAGTTCAAACGTACAGAGCC
AGTAGTGTTGGAACTTGGAAGTCATTATCTTTGAAGTTGTTGTCATAGTTATCAGATGTG
AATGTGGAATGCTTGCAGGAAAATAATATTAGTAAACTTGAAAGAATTTACAATGAAAGA
TGATGACTTCACATGTATAGAAGGTAGCAGCTGAGTGAAAGAGAATCTCGAAGAATAAAT
AATGGATGCTCCTGAGTGAGCAACTATTTCAACTATTATGTGCCTTGGAGGAAACAGAAT
TACACACCAGCTTTGGGGGTAGAGGCAAAATTAAACAGAAACTACACTTTGTTGTAGTAT
GTAGTATGTACAATTTCAAGCATTCAAACAGGCAAAATTAATATAAGAATGAAAGAATCA
TAGAACATATATATAGTACTAATAGTAGTAGCCTGTTCGGATACAAAAGGTAGAAGGTGC
TGCTTTTAAGAGCTTTTCAAAACATCTCAACTGGAAAAAAGCTCTATATTTCCAGGAGAT
AAGTTTTCAGAAGCACTTATGACTTGTATCAAAACAACTACGTTTGGCACAAAAAAAAAA
ATCAATGCAAAAATTGTGCTATATGGTATCGTCCCCAGGACTGGCTGTGACTGATCTCTC
TGGTCTAATCTCTTCTAGCTGCTGGAGCACTTGATGAACTTCCGGGCGTACTGATGGAGA
AGGATCAACACAGTGCAAAGCGAGCTTCAACGTGTTCAGCAACTCATCGCCAACTGTCGA
TGCATCTCTCATCATGTCTGCATCAAAAACCTCATTTGTCCACTCCTCTTTGACAATAGA
GGCAACCCACTGAGGCAAATCTAGTCCATTCATAGACACCCCAGGTGACTTCCTCGTTAG
GAGTTCTAACAAGATAACACCAAGACTGTATATATCAGTTTTAGTGTTTGCTTTCTTGAG
CTTTGAGAGCTCTGGTGCCCGGTATCCCAATGCTCCAGCAGTAGCTATCACGTTGGAGTT
AGCAGCAGTTGACATCAACCGAGAAAGACCAAAATCTGCAATTTTAGCATTTGTGTTCTC
ATCAAGCAACACATTGCTGGATGTGAGGTTCCCATGTATAATGTTCTCCAGGGAATGAAG
ACAAAACAAGCCACGAGTCATGTCCTGTGCTATTTTCATCCTTGTTGGCCAATCAATGAA
GGTTTCAGTTCCACCACCTGCATCAAGATGAACAAGAAAATACTTCACTTATTGATAAGC
TATATGAACAAAACAAATATGATTAAAACTAAGGTATTGTTCCTTAGGTGTTTTTGTTTG
TACTGTTCTCCAATTAGAATTATACATATTACTAAGGTAAGTAAAACTCCAATTCTAATT
ATGGAACAGCACACACAACACTTAAAGAACTATGTATGTTTAAGTTTTCTCCTATTTGTA
ATGTAATCAAACTTAAAGAACTATGTATATCTAAGTTTTCTCCTATTTGTAATGTAATCA
AACTTAAAGAACAGCACACCAAACTTACCATGTAGGAAAGAAGCAAGACCTCCTTTAGGC
ATGTAATCAAAAACCAGAAGCTTTTCCCCTTTGGGTCCCAAGTAATAGGCCCTCAGAGCC
AAGACATTGGGGTGTCTAACTTTTCCTAGAACACTGACTTCTGATTCAAATTCTCTGTGA
CCTTTAGTGATCTTTTCCCTCAATCTCTTCACTGCAACTTGGCTTCCATCCTCCAAAATA
GCCTTATACACTGTTCCATAGGTGCTCTTTCCCATGATCTCAGCAGTTGCACACAAGAGA
TCATCGGCTGTAAAAGCCAATGGTCCATCAAAATGGACTAGTTTCCCTCCAGCCTCCCCA
CCTGCTTCAACATCACCAGCAGAAACTGGAGGGACTCCTTTTTCTGTCCTTCCAGTGGCT
GCTCTCCCCGTGGCTTGTCCGTTCTCAGCCTTCGATGTTGATCTCTTTCTGATCAAGCAG
AAAAGCAGGATGCAACAAAGTATAATCAGGACTACTAGGAGAACTCCTGCTACTATGAGA
ATTATGTCTTTGGTACTTAGGTTCCTACGATGGTGCTGTTCTGACAGTACTTCTGGAGTT
GGGGCAATGACTCCTTGTGATGGAGCTTGTGAAAGACATGGGGTTGAAGGGCTATACCCA
CATAGTTGAATATTTCCCACAAATGAGCTTGAGTTAAATTTCTTGGCAAGTAGAGGTGGA
ACAGAACCTGAAAGGCTATTGTAAGAAACATTGAAGAAATCAAGACTACGTTGACTTTCA
AAGGAGACTGGAATTTCTCCACTGAGATTATTCAGTGACAAATCAAGCTGCCTAAGCATG
GAAATGTTTGCAATGCTTGAAGGAATATGTCCACTAAATTGGTTCCTACTCAAAATCAGA
ACAGAAAGATTACGCAATGTACCTAAACTTTCAGGGATTTGATTTTCAAGGAGGTTGTTC
TCTGCATTCAGCAATGTAAGTGAGGATAAATTAGAGAGGGTAACAGGCAAGCTCCCATTG
AAGGCATTATTAGAAATGTCTAGTGTCTTAAGCCTAGAAAGAGTTCCTATTTCATTTGGA
ATAGCTCCACTAAACTTATTATGACTAAGGGAAATCTCACTGAGCTCTCTTAAGCTACCC
AAAGAAGCAGGAACATTACCAGTGAAAAAATTATGATCTAGGATCAAATTTTGGAGCCTA
AAGAAGCCACTCTTGGGACTCCCACCCCAAGAGTTAGGAAGGTTGCCAGAAAGATTATTA
TTTTGAAGAGAAAGGAAAGTGAGAGAAAATGAGTGAGTTAGGCTAGTTGGTAAAGTACCA
GAGAAGGAGTTGAAACTCAAGTTAAGCCAATAAAGCTTGGTGGAATTGGCAAGGCTATAA
GGGATTGCTCCGGTGAGCAAGTTGTTGCTGAGGTCAAGAGACTGAAGCAAAGGACAGAAA
CCTAAAGAAGAAGGGATGGAACCAGTTAACCTATTGTTGAATAACTGAACCCCTCTAAGG
TTGGGAAGAAGTCCCAAAGTTGAAGGGATTGAACCACCAATTTGGTTATCATGAAGACTA
AGCTTCCTAAGGCCTTGAAGTTGGCCAATTTTGTCAGTGATTCGACCCTTCAAACCCTTC
CAAGGAAGCTGGATCACGATAACCTGTCCCTGAGCACACTTGATTCCAACCCAACCTCCT
GAACAAGCACCATAGCCACTGTCGTTCCAGCTCCGCAAGAACCCTTCTGGGTCCACCAAC
TCTTGCTTGAAAGCTTGAAGTGCTAAGAGGTTTGATGCTGTCACAACCACTCCGTCCCAA
CTTTCATCTTCACACAAAGCTGGTCTCACGCATGAAGGGAGCACAACAAGACTCCACATA
CATAAGAGAAACAAAACACGACATGGGTTGTTGTTGTGTGTCTTCCATCTTTCTTTCTTC
TTATCAGAAATACGGTTGAAGCATTGTGAAGTGAGGTTGGTTTTCTCCACTAGTAACATA
ACGAAAAAATCAAAAGAATAAAGGTAATGAGAATGAGAGATTTTGATTTAGTGGAACAAA
AAAACACTGTGTGTTGTAAGTTGAAGAATGGTTTTTGTAGTGAGGAGAGAGAGGAAGAGT
GAGGAGGAGCACGTGATGACATAAAGAAGTGTAGGTGCGCGCGCGGGTTGAGAGCGTGAG
AGAGAGAAAATGAACATGCGGGGTTCAGAAGTGAGGCTCTCTTCGTTGTTAAAGAAACAG
TGAGTAACGGCTAGTTTTCCTTGTAAATTGTGACTGTAGTGTGTGTATAGTTGTTTTTTT
CTTTATTCAAACAAATATAGATCTAGTCAAGAATGGAACTCATATCTCAAGAAATGAGTT
CAATTTTCATTATGAAAGAATATTATTGGTAAATAATCTGATTTTTATGAACGGTATTTA
AGTTTTGAGGTATACTTTAATTTTAGTAAACTCTGATATTTAATTTATCTAATTTTTTCT
TAATACAAAAGAGAAAAACAATTATAAATTGACCAAGATAAAAAGTTTTATAACATTATT
CAATTTGTTAACTTTATAATAATTACTTTAAAATTATATTAATAGTAAATTAAGATTAAT
CCCATACATGACCATTAAATCCATTAAAAAAATTGTTTTTGTTGGCAGGATTTATTATAG
ATTATGGTAAGCTCTTTTTTTTTTTTTCTTCATTGAAACGGGGTTAGATCCAAGAAAAGG
ATTACATAAGAATAGTTCTAGAGGATTCAATCACCAGAGATGCCTAAAAAAATGAGGTTG
TTGCTGGGAAACCATAGTGCAACCCTCCGTATTGGCGCCCTCTTCTTTTATTGCAACACC
CGCGCCTCTCCATCACCAACGGCTATTTTCTTCTTCACTACTTCACACTTAGTAGGACCA
TACTTCTTCTTCCTTGTTTTCTTGCTTCCACTTCCGCACACAACAAACACTGAAAAGCAA
TACTACTAGCTTTAATATTCTCACGGGTAAATTGTGTTTTCCGTCATTAAGATCTTGTCA
AAATTATTCTTAGTCTCTCCATTCTAAAAATGGTGGTTTTGGTTACCATGTGTTCTGAAA
CATCCTGAATTTAATTGAGATAAAATGAAGTATAAGATTTGGAACAAAATTCAATATATT
TTGAAAATACGAATATCAAAACCACTTTTTCAGATAGAGGAGACTAAAACTAATTAATTT
TAACTAATTAAACCTCGAGAAATCTAAAACACATTTTATTTTTATTTTTTATTTTTTTAC
AAATAAAATAACTAATTATCTAAAAAAATTCATTTTTTTAAAAAAGAAATTCACTTTGTA
ACAACAAGACAATAATAAACACATGTTAAAAGAAATTAATCTTAGCAATCCATCCTAACA
CGTTTTTTTTTACACACTGCGAAAATTATGTATGTCTCACACTCTCACTGAGTAAGTAAG
TAGAAAATACATGTAGGCTTAAATAAATAAAATCACTTTCAAACTTTTATTGTAATGTTT
TCATTCTAAAATTAGTGTGGCTTATTTGAATTTCAATTGATGAAAGAAAAATGTGCTAGA
AAGTGAGTTAAAAAGAATATAATACTAACCAGACTATCACTCCTCGAGTCTCCGTGCATT
AATGATTATGTATGAAAAAAATGTTATTTTTTTAATTGATGAATTGCTCTGACTCTGAGG
CCTTGGCTGGCATCTCCAAGTTTGACTGAAGTGTCACGTGGTGGAAGGAAGGAACTAACT
GGGTTCTGTCAAAGACACTTGTTGCAAGAGAAATTTATCAGAAACAGGCCTGGTTCTGGA
TATAAGACACAGGCTACATTAACGTCCAAAAGAGAGGTAAATGTAATTGTTTGATGATGA
TGACCCCGGTTCCTAAGAGTTGGTAGAGTAGTCGCAATTCATATCATAGCGGCTATGAAG
CACGGACACTTCAGCCCCTTATCGTGTTTGGTGTTCGATAACATTCATGTCAGTGTCCGA
CACCAACACGACACTTATACTACGTTCTATATTTTGAACATTACAAATGTACACGTGTGT
CTGTGTCCGTGTCGTGTTCGATGTCCGTGTCGGTGTTGGTGCTTCATAGCATAGGGGTCC
CAAAATCTGAGAAGAATTGTCGTGATCATCATGTTCATGGTACTTCGTTATTGGTCCACA
ACATGTTTGAAATTGGAAGAGCTCATAGCATTAGCTCATCCATCTCCTTTCCTTTGACAC
CTTTGTTCGCGTCGTTTTCTGTAAGAAGAACAACGCTCATTATATTCTGTGTGACACTAA
ATTTTGTGGGGTTTTAGTTTTGAATTTTGACCTCCCATACAAGATTTTAATTCATAATCC
AATGCTTGTGCTAGGAGTTGTATTTATTTATATACTAGTAGTTTTTATAATGTTGTAGTT
TATTTCCCTTCGTTATGTTCATTATTTTATCTTATATGTATATATTTTCTTTTCATTGAA
AATGAAATTTCTCAATGCAAATACTAGTAAAAAGCGTTATTTTCTCAATGATATACACCT
TAAAGTCTCACGTTATTTTCTCAATTCTCATAAGCGCCTTTCAAGAACAAAGCCGGCCAT
TAGACTCATACCAATGTCAAAAGCGAACAATTGTGATGCAAATTCCACACAGCCATTATC
TCTTCTTTCCCAATAGAATTTTTCCCTCAAAGATATAAAAATCTCCATATCTTTAGAGTC
CATTGGCCATACTATTGTTAAAGCATATCACACAATTGAATGTTTCTTACTTTCACACAA
TTTTATCTCCCCATATAACTCTTTTTATTCTTTGTGTCGGGCCAAGGATGGTAGTTATGA
GTTATGACCACAAGCTAGGTTAATGAAACTGATAGTTATCACTTAGAAGACATTAAACGA
GTGGACAAATGACTTGGGCAAATTAAGTGACATGGGTAAAGGCTGTCACCATAGAAGGTT
GAGGTGTGTACACTAGAAATAACCCAAATTTAAAGACTGATAATTAGGGACGAAACAAAA
TGACGAATGCATTAGATAAATGGTTACTATCACGATATTAATCAAGTTTCGTCTGCTGGA
AAATAATATTCTTCACTAGGCCAATTTCAAACTCGTGGGATGATTAGGATCACAATGAAG
GAAAAACCAACTAAAGGAATTGTTTACGCCTAGAGGTACTCCTCACCATAATGTAAGTAA
AAAATTATTAATTCACACTAGTTGATAATATTAGTTAATCTTATTTAAAAAAATAAACTT
TATTTTCTAATGTATCTTTCATGTTAATTAAAAGAAAATTTTAATTTTATAGAATCTTAT
TTTTTAAATGGAAATACTTTTGTTTTGAAAGTCATGTTGATATCAGGATAATATAAAAAA
ACTCTAAATTGAATAAAAAATATTTTAAGAATAATTTCATTAAATAAAGTGAAATCAATT
TGGTAATGTGTGGAGTGTGGAGGGACTCTGCCTTATCTGCATGGTTGGGCTTGGATGATT
GGTTTGATTACTACTTTACATTATTCTGTTGGCTTAAAGGAAAAACACTTTTTTTTATAA
CATTTTTCACTTATTTTTAAACAAACAAGTTACTATAATTGAAAAATACATCCAATATTT
AAGGTATATATAAATAATTTATATTTTTAAAAATTAAAAAAAGATAAAATATACATAATA
AGTAATGTGCTCAAAATAAATGAATAGCTAGCTAATATCACTTATCATCAAACCTTATAC
ATAGTCAGCAAAAAAATCAATCAACCCTTACATATGCATATGTGTGTACGCATGATTATA
AAACCTTACACGTAACTCAAATTAAACTTGCATGTTCCATATATCTTAACAATTAATATG
AACTTATTCACCCTGTCTTATTCTTAACCAGATCTCAAATTCCCTTAGAAACAAAATTTG
AAACCTGAATAATATGGACAGAAATTTCTTTGAAAGAGATCATCTATTACTTTTAAACGT
TATCAACTAAAAATTTGGTGGCGTAAACATTAGACTAACTAACTAATTACGGGCTCTTTG
GTTTATCATTGAGATTTGTATTTTTCAATGCTAAAACATGAATTCTAATGTAATTTTTTT
CAAAACAATTTGTTCTCACTTGGTAGAATTCTGTTTTTAATATTGGACTTAGATTTAATT
TTGCTAGGTCTCTTTCTTTTTTCTCGTTTATTTCATCGGTATTCTATTTTTAGTAAGAGA
TGGCTTTATGTGAAAAAAATAAAGGCAATTAAGTTATAAGTGGAATATTACTGGTATTTA
AAAAAGTAACAAAAACTACATTTTGTTAACGAGAATTAATGAATAGATAAGGTTTAAACT
TATGACTTTGTTCAAATTACTCAAATTCCTCGCTATCCCAATTTTAGTGATTTCATAACT
TTCTCTTTACGTGCATGTAATTATCTCTGCCTAATGAGCTACGGTCTGTCAAACTGATTT
GATATTTTTCTTACTAATAATTGCCTGTGCAAAGATCTTTCTATCTCAATTTGTTATCTA
TCTCGGTAGGGTCGTCACTCTCAATGCCAACAAACATGTGAAAAGAAGTTAATTAATGAG
ATCCAATAAACAACTTACCAGTTAATTAGCTCTAATAATAACAAACATTTATCATACACG
AATCTTCATCTGTAACAGAATATCTTATTCATGAAGCATTACATTTATCTTATTACTATG
ATTTTCATATACATATGGTATATTATGTACGTAAATGAATCAGTGGTCCTTTTACAATCA
AAAAGGTGACAGTGACAAGTACTAATGAGGCAACGGATGCTCACTGTAGTGATAACTCTC
TTTGGTAAATTAAAATTGAAATTACATAAACTACCAACAATAAAAGATATATTTTTTTAT
TTTAACATTATGATCCGTCACGAAAAAGTAATTCTGAGTAATTTAAAATTAGATTGAAAA
AATAAAAAGAAAATTAATTAAGAATAATTTTCATCAATAATCAAACTCAAATAATATTTA
AACAATTTAATTATAATTTTAACACATTAATCACATCACATATTTACTAATAATTAAACG
TATAATTTGTTTACTAATAATGTAATATGGAGAAATCAAGCCTATATTCACTCAAGATTA
TTTCCTTATTATAGAAATTAAATACTGTAAGGCCTTTATAGATCTTAATTAAACACTTAA
ATGTGTATCATATAAGTTTTTATTTGTAAGTTGTTTTTATAATTGAAAAGCAAATAAGGT
TAAATTATTTTCATATAAAATTTTAATTTTTTTTATAAGTTATTTTGAAAATTTTATTAA
AATATATTGAAAAAAATTTATAATAAATCATAAACTATAATTTTTTAAGTTTTCTTAAAT
ACTTAAAGGTCACGACATAGAATAATTTCAAATAAGATATAAATAAATTCCTCCAAGCAC
ATCTTAAATCTACATTTTTTTTAAACAAACTTTCATCGTTAAAAGCAAATAATAATAATA
ATAATAATAATAATAATAATAATAATAATAATAATAATATATTTATATATGCTTTAACAA
TGATAACTAACATTTATCAGAAAAAAAAAATTAATATACTCTCAAGCTATTTTTTAAAAC
AAATAAAAATTACATATTATAATTATATAATTTCTCTATATTTACACTTTTTTTAGATAA
ACAAATTATAAAAATGATGTAAATAAGACGTATAATATTATTATTTTTTACAACAACGTA
TAATATTATTTATAACGTAGATAATGCGTTTTTGATATTTTATATATATGTGGTGTAATT
ATTTTCTTATCCAATAATTAGCAATCTTATCTTGCTTTTATCCATGGGAGCTAATTAACT
AATGGTACCAATCTTTTCATTCTTTTAATAATTATTCTTTCCCCACTAGAAGACTAGAAG
AGTATTTGCTAACTACATATATCATTGTGTAAGAGTCTACAAATATATGTGAAGTGAAGC
TCCACCTGAATGCACCCGAATTAGAGAGAAGAGAGAGAGAGAGAGAGAGTTATAATAATT
AGTTGCTTCAATTCGATATATATAAACATGGCTTACAACTTCCCTGATGTGTTTTGCTGG
ATTCAGAGTCTTCCACCAATCTCAGAATGGGAAACAAGTTCCATGTCCTTAAACATATGT
TCTTCTTCAAGCTCATCATGCCAACCACGTCTTAATCTCACCGTATCCAAAAACAATAGC
AATAATCATTCCTCATCAAACCTCTATTTTGTTATTATTGCAGACTGCAACATTCCCATC
CATCTTTGGACCTCTAAACCTTTCAAGCCAAGCAGCACCACTATTACTAATAAAACCCAC
AATAATAATAAGTTAATAGATGATGAAGAAACCATTTCCAATCTCTTTGTTAATTTCATT
CAAGCCATCCTTCTTTATGGCTCCAACAAAAACAGCACTCCCTTCCTCAGATTTCCAAAC
CTTGACTCCATCACTTCCAACAACTTTTCAGATGTTTTCAATCTTTCTTTCTTCACCCTC
TTGTTCCTAGTTTGCATATATGAAGCTCCTGCTGCAGATTTTCGTTCTGGGTGTATTAGC
AACTTGAAAGACCACTTGACAGGTTTTCAGTCAAGACAAGCATCACATAAGATTATGAAA
CTCCTAGGGTCTAACTTGGAAGAGCATTGGATGCGTTCATTGAACCTCGCAGTTACTAAC
TGGGTTGGGGAACTTGAAGCACATAACAACCCCTTCAGAACACCGTGCCCTTTGTTTTCT
TATGCATTTTCAACAATTGGGTTGTGGAAGGTGCAGCTTTATTGTCCCCTCTTGGTCATG
GATGTTGAGAATTCAAAAAGCAATCCAGCTAGCGAGAGGCTTCAATTCTCCCTCAGATAT
CACCATGTGGAAGGTGTTCTTCAGTTCAATCACAAAGTCTTGATCAAAGAGGAGTGGGCT
GAAATCATGGTGGACATTGACAACATAAGGTAACCACTAATTAACCACCAATATATTATC
CCATTAAGAGATAATACATGTGGAGCCAATAAATAAGCATCTTAACAAGACAAATAAATT
AGTCATTAATCATGCCCATATGTTGTTATTAGAAGTTTAGGAAAAAATTCTCATCATTAG
TACAATCTTTTTTTTTTTAATATTTTATACAATCTTTCTCATTAGCTTGAATTCTTTAAA
AAGAGGTACTTTTTTTTTTTACTTTTCTTTTCTCTAAATTGTTCAACTTAACTAAATTTC
TAATAGTTCAAGATTTTGGTTTAGATTTGAAATAAGTGAAATTCGCTTAAATTTTATATT
AGAAAAAAAAACAACATTGTTCCATTTCTTTTTATAAAAATTGACGATCAAAATTACAAG
GTTCTTTTATGAATCACATTCATCCCTATATATTTATTTCGATATATTGAAGTGATTAAC
CGAATTAAATAAGGTTATTTGATCCATATAGCCAACGTTATATTATAGGTTTTGTTATTG
CTGTATATATATTGAGCATTTTTTTCTTATACATGTTTAAATTAATTTCCTTTATGAATG
GCAATCATCTTTATGTGGTTACATACTTCGCTATATTAATCAAGTCAAAATGATGTTATG
TGATCCATCTAAATTACTGTTATTATTATATAATGAGATAAGATTTTGTTATTATTATAT
ATTAAGTGATTGTTTTTCTTAATCCTTTCTAATTTAATTTCATATATATTTAATTTACAT
TAATATTAATTAACTCCTTGTGGACAGGTGTGATGTTATAAAATTGGTGAATGAGTCCCT
TATGAGCCAACGAGGGGTAGGTGCAGCTGAAAAACATTTTCCTTCGAGAATATCATTGCA
ACTTACACCAACACTCCAAGACCAAGTGTTGAGCCTATCAGTTGGCAAGTCCTCAGAGAA
CCCTCGAAAAGAAATTGGTGTTGACAAAAGCGTGGAAGCTTCATTTGAACCATCCAATCC
CTTGGCACTTAAGGTGTCAGCTGGAGAATCCTCAACCGTTAGTTTGAAGCCTTGGAAATT
CGAGGAATCTGTTTATGGATACAGTGCAAATTTGAATTGGTTTCTTCATGATAGTGTTGA
TGGCAAAGAAGTTTTCTCATCAAAGCCTTCAAAGTTTGCAATGCTTAACCCAAAGTCATG
GTTCAAAAACCGATACTCTAGTGCTTACCGACCTTTCAACAAGGAAGGAGGGGTTATATT
TGCTGGTGATGAATATGGAGAAAAAGTGTGGTGGAAAGTGGACAAAGGAGCCATAGGGAA
AACAATGGAGTGGGAGATAAGGGGTTGGATATGGTTAACATATTGGCCTAACAAACGTGT
AACATTCTACAATGAAACTAGGAGGTTGGAGTTTCGAGAAATAGTCCATCTCGACGTTGC
TTAGCCTTGCCTCAAGATCGTAGTTATGTGCAAACTATTTGTTTAAACGCGTGATTAGTT
CCTTAATTGTAAGATAGAACTTAGAAGTGATCCTTTAATTAGTTTCTGCAGGTTGATATA
TGATCAATGGATACGAGTATATATTATTGTCAAACAGACTACTTTTAACAATTTATTTTA
AATATGTATTCTTTATCATTGGCTTAATTAATTGTATGTCCATCCCATTAAATAGACTAA
TAGATTACTCCTAATTCCAGGCTCAAATAGTATTATGAAAAAAAAAAAATTCATGTGCTA
AAAAAGCTGCTATCTACGACATATGTTCAACAACGGTTACTTAAAAATGTCGTTATTTAA
CATTCAATGGCATTTTGGTAAATATGATATCATTTTAAAGTCGGGTTTCATAAAATCGTT
TTTGAATAATGTAAAATAAAAACGGTTTTTACGAAGTCGTTTTTGTTTGTGTTTAAATTT
GAAATTTTTTAAAAAATTAAAAGCGATGTGTCCTTTCGTTTCGCATCCTCTCCTCTTCTC
GATACGCCTCTCTCTCTCTCTCTCTCCTTATTCATGTGCGTGTTTAGCACATCCGTCGGT
CTCCATTCTTCAATAAACAAAAACCAGAGAGAGGAAGAGAAGAAAGCCTTGATGGGAGAA
ATCAAGGAGGAGTGCACGAGCCTCGACAAGAGCGAGCAGGAGAAACAGTTGCATAGCAAG
GTCTCGAAGGTGGCGCTATACTCGAGCGGGGAGAGCAAGGCAATCGCGAGTGCGTGCGAA
ACGGCGTCATCATTTGGAAGCGTGGGCCCCAAAGTGTCGCATCTCAAGTGGGGGAGGTGG
TACAAGCTCCGAGAACTCGAAGTCGCCACTAATGGGTTGTGCGAAGAGAACGTCATCGAC
GAAGGTGGCTACAGAATTGTGTACCATGGCTTGTTTCCCGATGGCACCAAGATTGCTGGA
GACCTGGATCTTATTTGCCCAAAGGTATTTTTTATCATAGGCGTAGATGTTTTATTTCCC
TATTTTGCTATTTCAAGAAGTGAATATTCTTAAAGATATTTTGTGGTATTGTAATGAGGT
ATTTGGAGCACCTGGAGGCTCACATTATGCTCATTATCATTTGGTTGGAAGAAGGACGGT
ATGAAATTATTCCTTTGGTTGTTGATGTTCATGTTCTTTTAGATTAGGAAATTGTCAACA
AGGAAAGAATAACATGCCGAATTTTATATTGTAATTAATTGGTTATGGCCTTGTTGGTTG
TCCCATACAATGGAAGACTTGAAAAGATATTAATTGTTTTCATGAAAATATATAATATAG
GAAGATGTGTAGCTAATGGTTGAAACAGGGCTTGATGCCTATAGATTTTCGATTTCATGG
TCAAGATTGTTACCAAGTACTATAATATTGAACAAGCTCCCCTGCAAAATAAATTTGAGA
ACTCCTACCTTTTAGTTTTTGTAGCCTCCTATTTTTTCAGAAGCAGCTCTGGACTATTCT
ACAACTGCAGTTAAGAGATTGGAAATTACGTGTTTTTGGACATGGCTACCAAAAATCCAC
CCTTCAAGGTTTGTGGCATTTTCCTCTTTATACATTTCAAATCATGTACCTACCTACCTA
CCCATAAACGGTGCAAAGTTTATTGATAAATAAATAACTGAATAACTGTGTGAAATGAAC
TCTATTTTTCATATCTTGTAACTCCTTAATGGATGCTTTATTACATTCTTATAATAATTT
CATGTTGCAATGTCTTTTCATGTAATTGATAACTACCCTATTTTGTTTTGCCTTTTTTTT
TAAGTAATTGAAAAAGATAAAGCAATTAATGCAATCTGAGTTTGTTATCTTTCAATAGAT
AATTCTGGGCTCTTCTTCAAAGGCCCGTAGAGAAATTCTTGCTGAGATGGGATATGAGTT
CACAGTAATGGTATGTGTTAGTGTTATAATGTAATTGCTTCAGTTTTGTTACCATTATTC
ATTAGTTTGATGGAAAGATTGAAAGAGCTTGGCTTTTAGACTGCAGACATTGATGAGAAA
GGTATTAGGAGGGAAAAGCCGGAAGATTTGGTAATGGCATTAGTTGAGGCAAAGGTATCA
ATGTCTTCATTGGTTTCCTATTATTTATTATTTATTTTTTAATTATGTGGATACAAATCT
TACAATCACTTCCTCTTAGGATGCTGTGGAATAATAACTATTTAAGAAATCTTACAATCA
CTTCCCTCAACCTTATTGGATAATTTCTCCCCATAATACATACATAAATAATATAAATCC
CATTATATCTATTTTAGAGATGTTCACAAGTCAAAGCTCTATGCAGTTACATTACAACAT
TGTTGATAGATTAAGTTGCAAATTTGGTTTATTGTTCTCCTATTTGGTAAATTGTAGTTG
TAGTAATCCAATTTCCTAGGCTCTCCATATGATTTTGGGCATCTTTTTCATTTTTTGGGA
TGTAAAGGCTCCAAATACAAAAATTTTAAAAAAAATTAGCATTTCTACATCGGTTCTGGC
ATGACCGATGTAGAGATGCTCAGCATTATACATCGGTCTTTCAGTTGTGATCGATGTAGA
ATGTTTATCATTTTACATCGGTCTTTCAACGACCGTCGTAGAAAACTTAGCATTCTACAT
CGATCCTGCAGTTGTGACCGTCATAGAATGCTGATTTTTCTACATCGGTCACAACTGAAG
GACCGATGTAGAATACTGAGCATTCTAAGACGATTCCTGGCAACCGTCGTAGAATAAGTG
ATCACTTTTAACGTTGTCTCCTACGATGACGGTCGTAAATCGATGTAGATAGTCTCCTAT
AACCGATGTAGATTGTCTTTTTTCTAGTAGTATAACTTTTACAATATGATTACTTTAAAA
GTCATATCTAGTAATATATGTTGTCAAAATGAATCCAAATTTTTAAATATTAGCGCAAGT
GAGTAAAAAAAATTAAGAAGTGCATTCCAAAAGGTAAAATGAAATGTATCCTTTTTGTTT
TGAAACTAAAAAAAAAAATGAAATGTATATTGGTTCAATGATTCTTGCCTTCACGGAGAA
CTCTAACTCTACTAACATAATGAAAAGGTAGGTAGAGAGATGATATAATTAAAATTCATC
AAATAATATCATAAATAGAGTTATTCACCTTCATTATAAAAGGAAGTTCTTGAGATGAAA
ATAATCATCTAACCTAAACAAGGAGATACCATTACCCAAGTGTGTGTGTGTGAGACAGAG
AGAGATAGAGAATATTATGAAGGAATGAGTTTCTCATATTTTTCTAGAAAACTTCTAATA
GGGTTTGTTTTCCCAATAGTGATATCAAAGTTGGTTGGTGCACTCAAGTGACTAGTCAAA
CAAGTATTAAAGCAAGATGAAGCTAATATTGGATCAATGTATTGAAGTTTTCTTAGCGAC
GTAGAACTCAAGTGGTATGTCTTATTAGTGTAAGAAGAATATTCATGGTGAGTAGGTGTT
TGTGAAGATGCTAGGATTGATGTTTGGAAATTTTAATTGAGAGAATTGAAGGGGTTATTA
GGAAAATCAATATAATTTTCAAATACTAATATAAGCGAGGAGTGAAAAATCAAGAGGTGC
ATTTTTTGAAGTAATGAAATGTATTTTGACTCTTGAAGAATCTATTACTATAATGAAAAA
GTAGGTAGAGAGATAGGATAATAGAAATCCACTAAATAATATTCATGAATGAGTCATTCA
CTATCACTATAAAAAAAATTATTGAGATAAGAATCATCATGCTCAAATAGGAATTCACAA
CCAAAGTGAGGGAAGAGGCTTGTAATAAAGAGTTTTTCAATTTTGTCTCTAAAACTTGTA
ATAAGATTTGTTTCCCAACAATATACTTCCCCTTGTCTCAAATATAAGAAAAATATGGCA
CATGATAAACCATAATTTGAGTGATATTTTATGTGAGTTTTTGTGCTATTTCATGTATTT
TCTTAACAAAACTCATATTTGGACATTAGTTTTTACCTTACATAAGTACAATTGTTCAAT
CAAGTGAAAAAGTTGAAAAATGCATAAATTGATGAATATTTGATACTATTTTCACTTGAC
TACGACTGTGATACATTGACCACAGACGTGATCAATCAAGAAGGTCATGGTCAATCATGA
GGGCTTCTTCATTTCACAACATCTCAAGCTTCAACACCAAGATTCTGATAACGACCGTGG
TGGATATCATCATGACCATAATGAGCTTAATTAAGGAAATTATCTTTAGGAGTCTAATTA
AAGTAATTATCTTTAGGAATCTAATCACAATATTTGTCTATAGTGCATCGAATTAAAGTG
ATTATCTTTTGAGATCATATTAGAATATTTATCTATAGTGCGTCTAATTAAAATAAATAT
GTTTAAGAGTCTATAAATAAGAAGTTGGAGTCTATACTTGGGAGATTTGAAATTCATTAT
ACCTTTTATGAATAACAAGAGCTTTTGAGAGGAAAACTACACCGAGAACAATAGAGGTTC
AAATCTGTAAGGGTTTCAATCTCTTTTATTTCTAATGGAATATTGTATGTTGTTTAGTTA
TTCTATGACCATAATTGGCTAAACCCCTTAAAACTCGGATTGTGATGTAGTTATACCCAT
GTACTCTAGTTCCTCTTTTTAATAAAGTTCTTTGATGTTATTTGGTTAATTGTATCTTTG
TGTGTTTGTGATCATCTTCTTATAATTAACTGACCAATAGAATGATAGGGTTTATAAAGC
TTGCATGACCAAACTAGCGGTATGAATCCCATTTTTACAAACTTTTCTGTTATTCATCCT
TAATCCTAAATTAAATCACGAATGAACAATGCATGGTTGATTTAGGGGGAAGAGTATTCT
TAGATCTTGACTCATAAGATGTTACTAAAATAAGAGGTAGTAGACAATTAATTGGTAACT
GAGAGTTCTTAATTAAAATAAGAATTAGGGGAAAATGATACATATGAAATCATAATCAGA
GTCACTTTTATTCATACATATCTCTTCTTTAAAATTGTTATTTGTCTGTAGTTTTATTTT
CTAAACACAAACAATCTCCAACTCAATATAAAAATACAAAATTATTTAGTTTATGCAAAT
TAGATTATTTGGGAACACAACTAGTCTCTAGAGTATGATATCCAGACTTTCAAGTCCACT
ATTGCTATTGTATAGGGTATACTTGCCTTTAATGAGGACATATTTTATCCATCAACACAC
TAACTAAAAAAGTAATTTTTTTTCCAACTTACCTTTGATTAGAACTTGATATCAAGGATA
AGAAAAATATATGAAAACTAAAACCTCAATTAAATAAAGGATATTTAAGAGATAGTAACA
TTAAATAAGATAAAATTAGTTAAAATTTTCTTATATTAGAGACCAGAGGGAGTAATTTTT
TATTACTTCACGGTATAAAAAAAATTATAAGATAAATAGTCATTTTTAATCCCTAAAAGT
GTAGGGCACAGACAAATTTGTCCTTAAAAGATGAAAAATTAAAATTTAGTCTCTGGAAGT
GTAAAAAGTACAACAAATTTATTCTTTCATTAACTTTCCTTCGTTAATGTTAATGGATCT
GCCTACGTGGCACATTAGGACGAAAATATCAAAAAATGTTTGCTCATGTGGCCTTCATTG
CTTGGCACTGTCTTTGTCGAGATTCTAATATGTGTTGGTGCGGATTGGGGGCCTTACCCT
CCATTTCTCAGATCTGGAAATCACAACCTCCTTTTCTTAAACCAAAAAGAACACATTGGT
GTCATCGACACTCTGCGACAACCACCCTCCGACGACATTGGTGTCATCACCGACCCTAAT
CTATTAAAACAATAAAAAAACCAATTTGAGATATGTACAAACAAAATGAAAAAAAAAATA
CAAATAAATAACAAGATCAAAACCCCAAGTCACCACTGTAGGTGTGACCATGATCATTCT
CAGTTTGCTGCTCTCGCCGTCAACAATCACCGATGTTAAGCGCTTTTTCTCCTTGCACTA
AGCCACCACTTCCTCATTTATCATCTCCAAATTTTGTAACCCTTAGTCGCTTGGTTTGTC
AGTTATTTTTATCAAAACACTATCTACGCCAATAGCCTTAATCTCCTCATTATCCTTATC
ACAGATTTGAAACCTTTCCAACCTCTACCAACACCACCATAGATATGAAACTCCCTACCA
ATTTCTTTAAGAACAAGCCCATAGCAAAGAAAAATCCAAGCTTCAAGCACTAGGTGATAA
AAGGCCCGACAATAATGGTGATTGTGACTTCGTTGTGGTTTGCACAGTCCGGCCACACCC
TCTATGTCGCTTCCATCCTTCGCTTCGACTACTTGGATCTTTGATTTTGGCTTTCAATTT
TTGGTCTCATATTTTTGTTGTTTGGATTTTGGGTCTCGAATTATGTGTTATCCTCTATTT
TGGGGTTTCAATTTTAGGTCTCAAATTTATGTTGTTTGGATTTTGATTGTTTGCAGATTC
AATTATGGTTACAGGAGGAAGGAAGAGACACCGTAGATAACAACGATGCCATTTTGCAAA
ACCTAATTGACGATGAAGTCATATTTGTGTTGTTTATGTGAGGAGGGTGTTGTTGAAGTG
AATGGGGTAATTGACGAGGGTGTAAGAGAAGATAGGTCGAAGCTTCAATTGACGTTTAAT
TAAGTACAAACTTTCTATTGTCAATTTCATCCTAATAGACACGCCGTCAACATAGGTGTC
ACGTAGCCACAATACTTAACATTAATGGGGAAGAATTAACGATGAAATAGTTCTATCACA
CTTTTTACACTTTGCGACTAAATTTTATTTTTCTTTCAAAGAAAAATTTGTTTTTACTTT
ACATTTTCAGGAATAAAAATAACTATTTATCCAAATTTATATTAACTACGTGGAAACTAC
TAAATTAGACTTTTTAAAACTATTTATTATTATTATTATGATGATGATAGTCGAAGAAAC
CTACCCTGCCTCGAGGGGAGGGTCCCACGTAAAAAGAGGAGGCAAGCACTAGTCACACAC
AGGCGCACGTTAGCGAGGAAATGTTCCTAGATTGAAACGGAGAAGGTAATTAGAGGGGCG
GAAATCTCAAAGTGACACACCTCTTTCCTCCTTGGCTTAACTCAATTCAACTCAGCTATT
CTCTCTTCTCTATAAGTTCCTCTCCTCCATTGTTTTTCCCTCCCATTCTTCCATTTCTGT
TTTCTCCCATCCCTCTCGCGCGCTCTCTCTCCCATGGAATCCTCCGCTGCCATTCGATCT
TTTCACTGTATCTCTCTCTCTTAATCTGTATCTGCATTTTTGTTATCAACGTGCTTTGCC
GAGTGTGATCTCTGCTCTTGTGTTTGCGATTTCTGATCTCGCCGTCTCGCTTTTCTATTT
TATTTGCTTTAATTTATGTGACATATATGAGGGGAAATCAAAATGATTGAGTATATACAC
TGTCTTGTTTTCTGTTTTATTTGCTTTAATTTACGTGATTTTTTACTTTCGAGCTAGAGA
GACTGAAGTAATGGCGAAGAAGCTCGCTAGATTTGTGATTTTGTTACAAATATTTAATGT
TTGTTGCGTTTAAGCTCTTTCCGCATTGTGTGATTTCTTGAAATTTCATTAGTTATATGA
AATGTTATAATCATTTTGAATTGAATGCTCAACAACTTAGGATGGAGTTTCTGCTTCTGC
TGATTTATTTGTACTTTTTTACGAGGAAATTGAAACGTGTCTACTGCAAAGGATTGTAGA
ATTTTTTATATATATATAGATTTGCACAGAACAATAACATTGAAACGTTTCATCCCGAAG
CAACTAGTTGTATAAGCTTGCTTGACTTGTTCCTTATTTCTGAAATGTACGAAGCTTAAT
TTAGTCTACTCTGTATGCAAAAAGAGTTGAAGGATAACACCAAAGAAATTAACAGAAAAA
TGAGGTAGCTGTGCCAAGTTATTTTATGCTAATGCTAAGTGTTGCTTATTTATCAGAATC
ACGAGCATAACATTTTCATGAAGGCAATTGAATGTATCCTTCATGTTTCTACAAATATAT
AACCAAAGGATGCTCTGCAATCACCCAAGAGCATGTAATTGGCATTCTGTTGGGTCATAT
AGTAGGACACAAGGGACTGTTTTTTTTTTTTCAAAAAAAATTATTATTTATCAACTTTGG
TGATACCCATAGGCTGTATACTATTTTTTATTGGAAACACTTGTCAGAAATATTTGTCTT
CTGTTGGTTATTGTTTTCTTTTCTTAATCTATTTCTGTTAAATAAAACAATAAGTTCTGT
TCTTTTTCCCTGCCTTTTTTAAATAAGCTTACTTCTTTTTGGAAAGATTCTTAATGCCTT
CTTAAAGGGTCAATTTTCATAAATCATAATACAATATTTTTGTAATACTTCAATGTTTTT
TTATATTTTTAAAGGGGCAATTTTACCATTGCAACCAGATGCTTACTCATGACTCTTGGA
TTTCAGATCCCATAGGCACTATGTCCCATGTGCGAGCCTCCCTTGAGAAGCAGGCTGTAG
TTCCCATTCATAATGCCGGATGGAACTCTAAAAGTAGGCTTTTCATCCAGCATTTGGCAT
ATGGTCAGAAGCACATTAATTCCCACACGAAGGGGAAAAACACACTAATTTCATGTGGAA
AAACAGCTGAAGCTATCAACGCATCCAAATCTGATGGTGGGTTATATTGATTGTGAAATG
AATTATATCCTTATGTTTTTGTTCTATTTGGCTATTGTTTGAATGAGAAATGTCTGATAC
TATTATTTGTTCTTAGTGTAATTATCTGCTTAGTTATAAATTTTAAGTGGTTGTTTTTAT
TCTTAAGATTTTGAGATACATTAACAAGTTGCAAAATTTTTGGGCTGCAATTTCATATTT
TTATATACATGATCAAAGATAATGTTCCTGAACCATTATCTACTTGTTGGAAAGAAAGAA
GATAGAAGAAAATCCATGCAAACAAAAGATGTCATTTTTATTGTTTATCTGTTTATTTTT
AAGGTATTGTGTTATTTACGTAGATACGTAATAGTAGGAAATCATTCTTCTCTGTTGCTA
AAAATTATTATGGTAATTGAGAAAGTCTGTATGAAGGTTATATATATATATATATATATA
TATATATATATATATATATATATATATATATATATATAGCTTTGTTATAGTTGGCTATTA
GAATAGCTTCCCTTTAATAAATTTTACTTTAACATTCGTATTACTTTACCTAACAATTAT
TTATGATGGTGCAGCTTCTTCAGATAACACTCCACAAGGCTCATTGGAGAAAAAGCCTTT
GCAAACTGCTACTTTTCCTAATGGATTTGAGGTCTATAGCTATTCTTTCCTTTTATTTAC
CTGCAAATCATATTTCTTATTAGTGAAAAATGATAGTTTGGTGGGAATTAGCAATAGTAT
ACTCATGAATACAAATACCTTATGTTACCTATTAGAAGAAATAAATACAAGGTATTTGTT
ACTTATGATAGCTGGTTTTTATCCAAGACTGCATAATTGCATTTGTTAACAGCCATGTTT
CAATGTGAATCTGATAAACCATTACTTATGTCTGGTTTGCTTATTATCAGGCTTTGGTAT
TAGAGGTCTGTGATGAGACTGAAATTGCTGAACTGAAAGTAAAGGTGAACTTCTTCTCTC
TTGTTTACTGCTTATGTAAAACTTGAAGTGCAGTATTTTAAATATAATAACAAGGATGAA
AAGAATCTCTGAGTGTATGTTTTAAAGCAAAATGTTATGTGGTAGGGTTCTTTTTGCAAT
TCGTAATTAGTGGCTGGATATGTCTATAGCCTGCCATATACTGTGTATTGAACTTATGTC
AAAGGATGATTTTAATTCACTTAACATATACTGGTATTTATGTTTTTATCAAAATGGTAG
CACATTGTAATTGAAACTTGAAAATGTGGTGCTTTTGTGCCTAATGTCCTATCTGAATAT
ATAATTTTTCTTGGAATTCAAATTTGATCATAAAAAGTTGTGTCATCTCAGCTTTGTTTT
TACACCAAATTGTGCTTAATAACTTTAATATAACTTCAAATCTGTAATTTCTGTCTGGTT
CATCAAAAGAAGCATGCTTTTAGCTGGATGACCTTGTACACAAACTTATTTGGCTTATAT
ATTGTTATAGGTTGGAGATTTTGAAATGCATATTAAGCGAAACATTGGAGCAACAAAGGT
TCCTTTGTCTAACATTTCACCAACTACACCACCACCTATTCCAAGTAAACCTATGGATGA
ATCAGCACCCGGAAGCCTGCCACCTTCACCACCAAAATCATCTCCAGAAAAGAACAACCC
ATTTGCAAATGTTTCCAAAGAGAAATCACCAAGATTGGCAGCATTGGAGGCTTCTGGTAC
CAATACCTATGTCTTAGTATCATCTCCCACGGTATGTCATCCATCAATAATATATGTTTT
AACTTCTGCTTTCACTCATTATCTGACTTGAGCTATGTAGATCACTAATCTTTCTTTCTC
ATTAGGTTGGCTTATTCCGAAGAGGTAGAACAGTGAAAGGGAAGAAGCAACCTCCTATCT
GTAAAGAGGTACATGTGATATGCCTGTCAATATCCATAATTCACTTGATACTCCCTCCAC
TGGATTCTCATTATGAACTCCACCATTGGATTTTGGTGCATGTGTTTGCGGTTCACTAAG
ATTTTGCAGTGCTGAAACTAATTGTTTCTAGTTTAGAGCCCAACTATGCACTGTGAGCTG
CCCCATGTAATACTGCATAACAATAGTTGCAGTCTATGCATCAGCCTGATTGCTTGTCGG
CCTAGTCTCCTGATTTGACCTTGCTGACATGCATAAGTTGGCTTTTGAAATAAATTTAAG
TATTTAACTGAAAAAACTGGATGCTATAAAAAAAGAAAAAGCTGACAATAATTAACAATG
ATATATTTTGGAACAAAAAGGAATATTTTGTAAACTTGTCAGTATAATATACTGTTAGAC
CAGGCCAGCAGGTTGAAATTGGGCTTAGTCAAAACAAATTCCCAAAGCCTTGGAAAGAGA
AGAGAATCAACCCTGATGAGCAGAGAGTTGGTTTGGAGGGAATTGCCAGGTAGGTGCTTA
GTTGGTGAGGATTGTGTTATATGGGAGGGGATATGGAGGAGGGGGTGTGCGCATTCTCTT
GGGTAGCTCTTGGGTGAGAAAGTTCAGTCAATCTATAAGGGGCTTGGTCCTGTGTAGTGT
TGAATGGTATGACACAATGACTAGTTGGCTTCTTCCTCTTTAGCTACTATAGTCTGCCCT
GTGTTTTAGTGTTTTAATCATACTAGGTAGCCATTTCTTCCAAATTAGTGATAATTTATT
GAGTCTATCAGATACAAATTAAATGTTCTTTGGTTCCCAAATATAGATTGACCTATATTT
TTCTGCTGGTGTAGTTATTAATATATTTTGTAACATACGATTGCAGCATTAAGTTGTTTG
AGGACCTCACTTTCATTGAACTGACTACAAATTATATATTCATTTCCTAATTGAATTCTA
GGGTGATGTAATCAAAGAAGGGCAAGTCATAGGGTATTTGGATCAATTTGGCACTGGACT
TCCTATTAAGGTGATCTGTCTCAGTTTCAGTAATTCTGGTTCCTTCCTAAATATTTACTA
CTTCTGACTAGTTTAATTTTTGGAATCTTGCAGTCTGATGTGGCTGGAGAAGTGTTGAAA
CTACTTGTTGAGGATGGAGGTATAATAATGTCTATTACCTGTTACAAAAATGCCTAGATT
TATTGACATGGTACTAAAGTAATACAGGGCTTCTGGGGAAAGAGTGATGATAGCATACTA
GCAGTGCTGTTCATCCAAAACTGAATTAGTTAATGCAATAGTGTGCCTCAGCAATAAAAT
ATGGCAATCCTAATAATTCTAAATTCTAGTAGTTTTTATTTCAAGTATTTTGAAGCTTAG
TTGCAAGGAACAAGCAAATTACTGGCAAGTGATAAGCATTAAAAATGAATTTTACCATCT
TGGAACACTTAGTACTAGAGCATTTTCTGACCTGTACTTATGATACTCTAGTATCATGGA
TGCCTAATTGCTTTATGGCTAGCATGCTGTTATCATATTAAAACATGGGGATGTTGGGTT
TGATTTGTTTAGTTTGAGCTCTGAAGCCTTGATACTCCAAAAATGGCAAAGTTTCGATGT
TGTATTTGTATGCAACACCGATACGCATAAGATACTCGTGTAGTTATCCAATTCCAAAGA
GTATCCTCTATTTGTTTTTCCAAAATTTTATTGACTGGTATGGTTGAGAACGGCTCCAGT
ACTTTGAATAGGAATAAAATTCACTACAGCTTTATCTTTTCCTATGTATCTTTAAAGTTA
TAGTAAATACTAAGCATTACTAGGCAATCATATAGATTCAATACTTCTTTTGAGTTTTGA
GTAGGACAAATGATGCACATTTTGACACAAGTTAGGGGAAGATTTTGTAACATGTTGGAA
AATAATACATTCTGGTATGAGTAATGGAAACATATATCACTGTTTATTTGTTTTTGTTTT
TTGTTATTATCATAGGTATTGTTTTTGTGAATTATGATCATCGTTCACTGAGATAGTTAT
GTATTAGTGAACGATAATCCATGTCTCTTAATAAATATTATGCATTTGTATAAAGTATGT
ATGTCTCTATCTATCTATTTGTCTGTGTGTGTGTGCATTTGTGTTAATGCATCTGAGTTG
CATTGTATCTTGAATTTTAAGAATTTCCTGTATGCCCTGAATTTGGGTTGTACCAAGTAT
CTAGGCTTCATAGAGTTTGAGAAAACATTTTTTGCTTTCATTTCTTGTTTTCTCTTATAA
TTAGAAAAATATCTTAAAACAGAAAACAAAGTGGAAAATAATATGACTTGGTTGTAATTA
TTAGTGTCAAGCTTATAAGAATCTTGGATCCATACTCATTTAATCATCATACCAGACAGT
GCGGATCTTGAATCCTGTATACCTTTTTATCTGATTTGCTAAATGATGGAAGGTAACAGG
ATATTCATGATAGATTAATTTTGCTCATTGTAATTTACAGTATATCGGTTTTCTAGTTAG
TCCTGACTATTGACTGAATGGAAAAATATTGTTTAGCTCTTAAGGTCTTAATTTCTGCTA
TGCCTTTTTATTGATTTATTGATTGCGAATTCCGTCCTTTTGCAATATTTAAACCTATTT
TGGCGCATCAATGATGGCAGTTACACTCATCCATAGCTGTGATGAATTTCTTACTATTTT
TTGTTTGCGAATTACAGAGCCTGTTGGTTATGGAGACCCTCTTATTGCTGTGTTGCCATC
TTTTCATGACATCAAGTGAGATAGCCTTTTCCTGTTAATGATTAATCATGCGGCATTTTT
TTTATTAGGGGAGCTATTCGCGTATTCGCAAAAGTTTTTAATAGGTTACAAATAAATGTG
ACTAAATATTTGGTACGGAGGTTCCTTATTGAAAATCATACTTTGTATACCAATTGCTTA
AATTGTACCGGATAAGCTATTCTGAGACCGCCTCATTATGTATCTTACTTGCTGGAGTTT
GATTACTAGCTTGTCCTTTTATTTTATTTATTTTCTCCCAAAACTATATTGCAGAACTTG
TTTTTGAGATTTGGAACCAGAAATAGAAACATTGCACTATTGCAGTGCTAGAATATATAG
AAATACTGTATTCTCAACAAAATTATGATCTAATAATCATTACTTATCACCAGATTGTAG
CAAATTCTTAGAGTTTCCAATCTATTCAGTCACATTCCCAAATGGTAAGGGCAGATTGTG
ATGAATCATGTTTTAAGGCTAAACCATGAGAAAGTGAGAAACCATTGTATCCCAGAAAAG
GGCATGTGAAATCTGTTTGTCCCTGCCCACATTTTATTTTATAACGCAAATCTTGACCCA
TCAAACTAGGTAGTCAAACATGATTATTACATGTAGCAAGATATTTGATGTGTGATTTAA
AACTTGAGTCTCACTATCAAACGTGGATATAATTTGAAGTTTCTCTTACTAACTTTGCAT
TAAAGCAAAATTCATAAGTTGAAAAGGTTTGATAAGATTGATCATGAACTTGGGGACCGA
AAGGAAAATGATACATATATTATTGCTCAAATTTAGTTAATCCTAATTTTGATACGAACT
TTAAATTTTAAATGTTGATTCAAATAAATTATTTGATGCCTTTTTCACCTTAATTTTTAA
ATTTTTTTATTCCTTTAGGCTATGTTTGACACATCATTTTATCTAATTTTTAATATTTTT
TATCAATTGAAAAAATTGTTTAATTATTCGGTAAACAAGTTTTTTAGTAGTTTTTATCAT
TTTTTGAAATATTATTTGAAGTAGTATTTTTTAAAACGTTAATTTCTAACTTTTATATTG
TATTCTTTTTTATCCATAAAGTATTTATTAAATTTTCTTATTATCCTTTTTAAATATAAC
ATGATTTCATTATTTATTTTTTCACTCATTCCTCTCACAAATTTTTTATATTATACTATT
TGTTTTAATCAATGTACTATTTATAATAATTTCAACATTATACTTCACAAAATTGATAAT
GATAAATATAAAAAAAATATTATTTTTTGAATATTTTTTACATTAAATGATTTATTTAAC
TATTATGCTAATTCATATAATAAAAGTATAATGTCTACTTAATTGTATGTTTTTTTAACT
TTATTTTAAACTTGGTTTAAAATATTTTAAGACATTATATAAGATTAATAGTATAATACA
TAATATGAAAAATGTAATAACTGAAACTAAAAATGTAATGAAAAAAATAATTTACCTGTG
GATAAAAAATTATAAAAGTTTAAAATTATTAAATATAAAATTATAATACATCAAAATATA
TTTTTATCCATAAAATGACATTGAAATTTATATAAATATATTATTCTACTCGGACTCGAT
AACTTTTGTATTTGGTGGTCGATCAAGTCAGCTACTGATATCCTAAATATCTGGGAGATT
TAAATAAGGCAAAATGGAAAATTTATCCCTTAGTCATAAGATAAAAATATAATCCTAATA
CCAAAAAATATAAGATTGAGATTAGATAATTCAATCTCTCCCTATATAAGAGACGATTGA
GACTTAGGTAAAACATTTTGATTTCAACTCATACATTATTACTCATATATCTCTCTACGC
CTCTAACTTGAGCATCAAAGTGTCTTTACAGGTACCTTCCCATCGCAATTCATGGAGAGG
CTTATGAAAGAGCACCATCGTGAGCAGGTCTTGTTGAATATGGTAAGAACATTTGGCGTC
CACCGTGAGACCAAGGAAAAATTTTCCTTCAACCACATCATCATCATTAATGGTGACAAA
CAACGACAACACCTCCACAGGCAACCAAAGACCACCACTGGGGTAGGTGGCCAACTTGTT
TGGCATGCCAGCCATGAGAATCCTTGAAAATGAGAGCCCAACAACAATGGTGAAAATCTT
GTGTCAGGTGCCTGGTGAACATTCCTTGATCGTTGCCTTGCAAAGGGAGATGAATGGCAT
GTAGTAGCAGAATGCCCAAGAGATGCAGGACCTCCGAAGGGAGAACATCGCCCTTAGAGA
ATAGTACCAAAAGATTCAGGAAGACAACAACGTGCACTCGGTCTCTGCAACCAGCACCCA
TCAAACTCATCTGCCAGAGACCGATGAAGTTGACATGACATGGAGGGAACAAACTTCATC
AGTCACTCAATAGGTGCATAGGGTGGAGAATCAACTCCATCCTTTCATTGATGGCATCAT
GGAGACACGGTTACCACCCAAGTGGAAGATGCTAATGGTAAATAGTTACGATAGACTCTC
GAACCTAGATGAACATGTAGATGCATTTGCCACCCAGATGAATCTGTTCACTAACGACGA
TGCCATCATGTGCCGAGTATTCCCGACAATCCTAAAGGGAGCAGCACTTTGTTGGTACAC
TCGTTTATCGAGAAATTCAATTAGACTAAACTTATGTTTGGCTCATTGATAAAAAAATCA
AAACTCGCGGACGTCTAAACTAAAGACCACTATGCGTAGTTAGCTCAAACCACGAATGAC
TTAACTAAAAATTTGGTTGTGAAAAAGACCAAAAGATCCAGTTACTTGGCTAAATCAAAA
GATTGAGTTACTTGGCTAAATTCAAAGACCGACACTAGCAGTCGACTTAGACCACAAACA
ACTTGGCTAAATTTGTGGCTGTGAAAAAGACCAAAAAATCGAGTTACTTGACTAAATCCA
AAGATCGACACTAGCAGGCAACTCAAAACAGAATTTTTTTACTTTTATAAGCTCTTCCAG
CTAACTCTAGAGCTTAGGGGCATATGTTGTACTTGAACTCGATAACCCTTATACTCGGTG
GTGGGCCGAGTTGACTACTCAGATCCTGAATATCTAAGATTTAAATAAGGTAAGAGAGAA
ACATTATCCCTTAATCATAAGATAAAGATATAATCCTAATACCATAAATTATCTTTTATT
AGAAGAAAGATAAAATAATTACCTTATATTTTCCTATTTTCATTATCTGAAAAGAAAATA
TTAAGATTAGATAATTTAATCTCTCCTCATATAAGAAACCATTGAGACTCAGGTTAAACA
TTCTAATTTTAACTCACATACTATTGCTTGCATACCTCTTTATGCCTCTAACCTGAGCGT
GAGGTGTCTTTGCAGGTACTCCCACCACAGTTAATGAAGAGACTCACAAAGGAGTACCAC
CGTAAACATATCTTGTCAAATCTAGTAAAAACATATATCTATTTTTTATATTTTACATTT
TTCAACTATTTCAGTAGACAATTCTACCGAATTCTTATAATTTAATACGTTAACTTAAAA
GCTTTCCGCTATCAACTAATTTTACCAAAAATAGTGTTAATTTATTTTATATAATAAAAT
AAAATTCACATGATTAACAATAAAGACATCTTCTATTATATTTTTTTCTTGAAATAAATG
ATAATATTTCATCATTATTTGACAATAATGATATTTTTATCTCTTATATATTATTTTGTT
TTAAAAATTCTCTAAATTTAAAAATATTCATAACACTTACGATTTCTTTATATCAACAAA
GAGAAATGTATATATAAATTATAGGTAAAGAGGTACCCAAACTCATATACATCCAAAGTA
CATTAAACACCCCCACCAAGCATGTTAGTAGCCATAAGTTATATTGTATTTAGCTGTATA
AATGACAAATAAATGCTGCATAGCCAGTGCTACTAAGAAAACCAGCCAGTGAGAATGTGC
ACCTAAGACCGTAAAAGTGTAAATCCCTACCCGACCATTATTATTTTATGGGAACTACCG
TTACTAACCCACGGAAAAATTGTTGTTTATAATTAAATCATTACTAAGTTACAATAAATC
AAATATTACTTTAAGTTAAAAATATAGTCAATTTATACTCCAGATGGTCAATCACTTCAT
TTGGTTAATCTGGTATAACTTTTTTTTTTTTTTTTTACTGAATTTGGTTTTAGATGGTGT
CGTATACTTGATTTGTATATATTTTTTATTTTTACTTTTGCCCCTTAAATATAATCAAAT
TACAATAGAAGCCTTGTATAGGCAATTAGGCATATACGGATATTGGATTGGCACCTGTGT
CACATCTGACTCAGATGAATTTTATCTTACATGAAAATTCCAAAAGAATGGAAGATATGG
TAAGGGATAATAAATCCACTTGGCGCCGTATCTGTAACTGTATGGACACGAATGCTTCAC
GTAGAGCTCTAAATTTGAGACCTTATATATACTTTCAATATTATTTATTTTTTTTGCGAC
CACGAAAAGTAGGAATTTATTATTGAACGCAGAAAAGGAATGTTGTATATTTTTATATTT
TAAATCTAGGATTTTATGAGAGGCAAAAATACGAAGAATCTCTCCATATGTTTTCTGATA
CACTTTCTCTGTTATATTTTTTTAATAATAATTAAAATTTATTAAAACAATTAATTTATA
TGGAAATATCATTAAATAAAATATGAAAATTCACAAAAAATATAAGTTTAATAAATTTAA
ATTAATAAAAAAATATTTTTAAAATAATGTTAAAGAATTAGAATATATTAGTAGTATCTT
TTCTAAAAGCACATGAATTCCACCTAAAAAATTGATAACTTATAATATCACGAACTAAAA
TTAATACTTTTCTTTTTCGAAAGATATCATTGTCTTTTTTATTTCTTAAATATTTCATCA
TGAGTTGAATCTATAACCTATGTTATTCGTGTTTTCTGAAACAAATTTTAATACCTCAAA
AAGATTATTTATCTTTTGACTTGGGATATCCTTGATTGGGAATAAAAACATGTCTTCTTT
ATTAATTTAGTATTTAATCATGTTCAATTGAAATTGTCTAGGAAGAAAAGAATATTTGAA
AGAAAAAACTTTTCCAAAATACACAATAATAATTTTATATAAAAAAATACATAATGATTA
ATACAATCTTGTAAGGCTGAAATAAATGTTACATGTTTTGTGTGGCAGAAAGAAAGTTGA
AAAAGTGATCTGAGAAAACTTAAACCTTTGCGGTTGATGACAAGCCCTGATTCAAGCGAA
ACTAATGAAGCCCCACACTACGGCTTCTTCTTTCGCCACTAGTCTCCCGCACGTGCCTTG
CTTCCGCGGCACCACTGCCGCACGTGCCACTCCATCGGAGCCTCATCATGACTCCGCCGG
AGGCCTCGAATTCCGACGCGTCTCGACATCCAAACGCCGCCTCATCAACCTCTCCGTCCG
CCACGCTAGTCGCGTCACCGCGGCGTCGAATCCCGGCGGTTCCGACGGCGATGGTGACAC
TCGAGCTCGGAGCTGCCGGCGCGGCGTGCTGATGACGCCGTTCTTAGTCGCCGGCGCGTC
AATCCTGCTCTCGGCGGCGACGGCGACGGCGAGAGCGGATGAGAAGGCGGCGGAATCGGC
TCCGGCTCCGGCGGCGCCGGAGGAGCCGCCGAAGAAGAAAGAAGAGGAGGAAGTGATAAC
GTCGAGGATTTACGACGCGACGGTGATAGGAGAACCGTTGGCGATAGGGAAAGAGAAAGG
AAAAATATGGGAGAAGTTGATGAATGCTCGAGTGGTATATTTAGGTGAAGCAGAGCAAGT
TCCGGTTCGAGACGATAGGGAATTGGAGCTTGAGATTGTGAAGAATTTGCATAGGCGCTG
TTTGGAGAAGGAAAAACGATTGTCTCTGGCTCTTGAAGTTTTCCCCGCTAATCTTCAGGA
ACCGCTCAATCAGTATATGGATAAGAAGTATAACAATGCCTTTCAAATTCTGATTAATGC
TTAGCCTCTTGCATCAACCTACTTGTTAAACCTCTTCAAATTCGATTTTAACATAAAACT
TTTAAATGTTGTTGATATTGCGGCAAATCGCGAACATTGATGATGCCAATCGCGGTCGCG
GCCAATTAAAAAACCTTGATGTTGCCGCCCAAATCACGGTCACATACCGATTTTAAAACC
GTATGACATCAATAAAACGTGATTTTGCCCGCAACATTAAGTTTATTCAAGTGTCTACAA
CCGCAATTGTGGCCGTAGAAAGTCATGTTAGCTAAATTAACAAAATTTCAATCAACAATT
ATAGAAGAGAGAACTTTTTGTTGGCAACCAATTTCTGTCTGGCAATCTTTTTTCTGGCTC
ACTATTTTCTGTTAAACTCTGCAGTTCTATTGTCTTAGTCAGAATTACTTTCCACAATTC
TGCATTACTTGTACTGAAACTTGACTTTCATGGCAGGATAGACGGAGACACCTTGAAGTC
TTACACGCTACATTGGCCGCCTCAAAGATGGCAAGAGTATGAACCTATTCTGAGCTACTG
TCACGAAAATGGAATTCGTCTTGTTGCTTGTGGTACACCACTGAAGGTATGCATAAATTT
CATCCAAACTAATGTTGATCTTTCAAATGTTTATTTTGTCTCTGAAAATTATGGTTGTGG
CTATAATTAGGAGCAAGGGGAAAATGTTAGTGTAGTAGATACTTTTGGTACTTGCCAAAT
TAACTTACAATAGGTGCAAAAGGTAGGTTCTGTAAAAAGTTGCCCTTAAGGATTTTGATT
GTGTTAGGCCTAGAAAAATCTAATCTACAAAGGACAGACCCTGAGTTGGAAATAAACTCA
TAATCCTCAAAGATTCCCTCCCATATTAAACTAAAAGTTAAACAAAACATTTAAAAAAAA
TTGAGGATATGATTATGAAAAGTTGCCTCAAGTGGTTTGATCATGTCAAGATATGAGAAG
GAAATATAATCTGTTAAAAAGGGTAGAGGGCAACCCGAGAAACTTTATTGCAATTTTTGG
TTTTTGATAGAGCTAAAAGTTTTCAATTGATCCATCTAGCAAACTCCACCTTGTAGGATT
AGGCTCAGTTCTTGTATTGTTAGGTCAAGACACATAAAGATGACAATTAATACTTGAACT
TTTCTCATCAACAGATCTTAAGAACTGTCCAAGCAGAAGGAATTCGTGGGCTTACAAAGG
ATGAACGTAAACTATATGCACCTCCAGCTGGTTCAGGCTTCATATCTGGCTTTACGTCTA
TCTCACGCAGATCTTCAGTTGATAGTACTCAAAATTTGTCTATTCCTTTTGGTCCAAGCT
CATACCTTTCTGCACAAGCTAGAGTAGTTGATGAGTATTCTATGTCCCAGATTATCTTGC
AAAATGTGCTTGATGGAGGGGTCACTGGTATGTTAATAGTTGTGACTGGTGCAAGCCATG
TTACATATGGATCTAGAGGAACTGGAGTGCCAGCAAGAATTTCAGGAAAAATACAAAAGA
AAAACCAAGCAGTTATATTACTTGACCCTGAAAGACAATTCATTCGCAGAGAAGGAGAAG
TTCCTGTTGCTGATTTTTTGTGGTATTCTGCTGCCAGACCCTGTAGTAGAAATTGCTTTG
ACCGTGCTGAGATTGCTCGGGTTATGAATGCTGCTGGGCAGAGGAGAGATGCCCTCCCAC
AGGTAAGCCAACAATTACAGTTACTAATTTGCTTGACTGTTACTCTTCTTGCTCCTTAGA
CCCTCCTTCCAATTTTTAGCCCTTTTTGTCCTCTCATTCCTAGTGGGATAAGACTTTGTT
GTTGTTATGCTACTAGGTGTCTTGTGTCAGTACCTATTATTTTTTATTTTTTTTTGTATG
CAATTAACTTTGGCATTTCAGGAGCATTTTAGAGCCCACTTGTTTCAACCTTTTTCATGA
AAAATCACTTCTAAAATTTCTTAGTGTGTTTGTTCATTTTTTTTAATAATAAATCAGAAT
CTATCTATATGATTATTTTCAGAAGCTACTTTGAGTATTAGCTGAAAAAAAGTCAAAATT
ATAAGTACTTTTTTATAATAAAAAGTATATTTTATAATTATTGTAATGAAATAAAATAAC
TACATAAAATATGTAAAGGAAAGTACTTTTTTTAAAAGCTTAAACAAATGGGTCCTTATC
TCTTTAATTGATATCTCTGTTTTGCATTTTATTGATTGACATAAATTATGCAGCTCAACC
TACTGCTGTGGGGGATTTCAATTGAATGTTTACAATTTTGTAGTGCTGTCTTTGTGCTAT
CTTTATTTTCCTTTAAATTCATATTCCTTTTGAGGTCAGATTCATTGCACAGAAATATAT
AATTTAAGGACTTAAAAACTTTCCAATGGGTGAATTGTGATAATGAGGTCTTTATTTCTG
TTTTCTTTCTCCTAAAATAAAGTACCTCTATTCTATAATTTGGCATGCATTAGTTGCATA
CTTGCATTTGATGATTAGGTAGTTAGTGTGTTTCTACTTTCTAGTTCTTTTTCCTAGGCA
TCTGTTTACTCTTTTATGTGCATGCCCTAGACAGGATACTGTGTGCCGATATTGGTTGTA
AACATAGGAAATATTTTCCTTTCTATTTCAGGATCTTCAAAAGGGAATTGATCTTGGTTT
AGTATCACCAGAGGTATTGCAGAACTTCTTTGATCTAGAGCAGTATCCTCTGATTTCAGA
ACTCACTCACCGTTTCCAGGTAATTTTTTATTTGAAAACGAAACCTTTATTCTGCTTTTA
ATCGAGATAATTTCATCACTTTGTTCCCTTCATATTCTTACCTATCCATCTGGTTCTTTT
AATACAGGGATTCAGGGAAAGATTGTTGGCAGATCCCAAATTCTTGCACAGATTAGCCAT
AGAAGAAGCTATATCGATAACAACTACATTATTGGCACAGTATGAAAAGAGGAAAGAAAA
CTTTTTCCAAGAGATTGACTATGTTATTACGGACACCGTCAGAGGATCAGTTGTTGATTT
CTTTACAGTGTGGCTTCCTGCACCAACTTTGTCATTCCTTTCATATGCTGATGAGATGAA
AGCACCCGACAACATTGGTTCTCTAATGGGACTTCTTGGCTCCATCCCGGACAATGCATT
TCAAAAGAATCCGGCAGGGATAAACTGGAATCTCAATCATAGGATTGCATCAGTTGTATT
TGGTGGTTTAAAACTTGCTAGTGTGGGATTTATTTCAAGCATAGGAGCTGTCGCTTCATC
AAATTCTCTATATGCAATTCGTAAAGTCTTTAATCCAGCAGTTGTCACTGAACAACGGAT
TATGAGGTCACCAATACTCAAAACTGCAGTTATATATGCATGCTTTCTTGGAATATCAGC
AAATCTCCGTTATCAGGTATGTAGAGAACTTGTCTGTTGCATAAAGTTTTCTTTGTAACT
AATATGTAACAATAGACATTATGGAAGATTGAACCTAGTTTCTGTAGGAAATGAAGGCTT
CTATATTGTCAGGTTGATGGTTTATGTTTCAATACTCAATAGTTGATTATGTGATGTCTT
GAATTTTTTCCTATATATATTAAAACCGGACAGAGAAATACAATTTGGAAATTCATGTAG
ACATTTTGAAAGCAGGACTTGACCAGCTGTGTCTTGAATAACTAGTTGTATACTATGTTT
TTCTTACCACTTAGGCAGTGCTTGAAGTAGATGGTGGGTAGCAAGCATAATAGAAAAGAT
TGGACAGTAATGTGGTATGCTTTTGGATCCTCTACAATCTATTTTGTTCAAATAAAGGGA
GTAGAGGGGAGGAACACCTGGTGTGGTTCACAGGGAAATAAAAGAGAAAATATATATATA
TATATATATATATATATATATATATATATTATTTTTTTTTATTATATCCTCATAATAGGA
TAAATCATTAAGGATAGTTTAGTCTAATTAGTGTAAAACTTTCTCCTCTCCCAAATTCTC
CTCTCCAAATTGATAGGGGGATTTTACTCCCCTCCTTTCTCCCTTTCCTTCCAAATCTTT
GCCCTCCTTCCCCTCCATTATTTTCCAACCAAATATTATGTTAATAAACCCTGGATGCCA
TTAAAAGTAATAGTGAAAAAAAATACTCAGTTCAATAATATCTATTATATCTGATTTTGT
AATTTTCTTGCCCTTTCTACTTCATCTGCACTTCGCTGAATAATTATTTGAGGCTCTTTC
TTTTTTAAAATTGGATTTTGATTCTTCTGTGACTACGTCTTTGGTTATATCATATTCAAT
AAATATGCATCCATAATGTCTGTTCTTTTGTATTAATACGGAAATTATAATATTTTGTTG
AGCAGATAATTGCTGGGGTAGTGGAGCATCGGCTTTCTGAACAGTTTGCTTCTCAAACAT
TCTTTGTAAATATGCTTTCTTTTGTAGCACGGACAGTAAATTCGTATTGGGGAACCCAGG
TCAGTTTTTTTATTTTTTATTATCTTCTATCTGCTTTTGTTCAGTTTCAATGCTCAATGT
TCACAATCTCTTCAGTAAGCGGATTTCATTTACTTATGATTGAACACTACAATCGGTTAT
CTTAACATCTAATATTTAACATGTGGCTTTTAATGCACGGCCATGTCAGCACATTTATTG
AACATTGGATGTTAAGTTAATTGATAGACCAAAATTGCATGATTAAAATAATTAGAGATC
AAAATTGTGGAAAAAACAATAGAGGGACAAAATTTGTGATACTGAAAAAGTAAGAAGACC
AAAAGTATAATTAAGCCAAAAAATATAGAGCATTTTATGAAAAGTACAACATGTAATGCA
TTACTTTTCATTTAATACATTTTATTTTTTTCGCTTAACAAGAGTCTTAAGGAATCTCAT
TAGCATTTCTAATAAGGTCTTGAGGAATTAATAATAGAGTTTTTTTTATTAGAAATTTGA
CTATATTATCCTCTTCCACCGTTATTGCTCTTTCATTTTAGTTAGTGAAACACCAAAAGA
CAAAAAAGCAATGTCGTCTCTATGTTCTAAAGTGACATAATTTGAAACAGACTAAAACCT
CAAGAATCAAGAGTAGCAATTGTTTTAAAACCAATGGAGTATAATTCAGTTTTATGTGAA
TTATTCATCTGCACATAAATTTCAAAATTTGACTAGTATAATTCTGTTTTAAAATTTGTT
ATTTTCGTGAATTGCTGCAGCAATGGATTGACCTAGCGCGCTTTACTGGCCTACAAGTTA
GAAAGACAGAGTCACCAACATCAGATACTCCGAATCCTGCTGCAATTTTGTGCAATGAAA
CAGAAGAAGCCAGCATTGATGAGATAGAAAAATAAATGCGGTGAAATTAGGTTGCAACAT
TTTTTTTTTCTTGGTAATTTTGTATTATTCTTGAAAAGCTGGTAAAATAGGCACAAAGGA
ATAGGTTCTGGTTTTCATCACAATCTGGTTTTCTTGGTAATTTTATACTATTCTTGTATA
CGTTTTTGATATTTCCCATTTCATAGTTTGGTGAATGTAATATTTTTATTTTTAAATGTA
AGTGGAATAAATAGAGGTGTCTCAAATCGAAACCAACGCAAGTACTCTTGGCTTTAAGGG
ATATATATCTCTTGGTAAGTCTTGTTAAAAATGATATAAGAAATCAGCAGTTGTACATCT
TATATAGTATTAGCAATTTATCATGGAACAATGAACTTTCTATAGATTTGCAGTAACATT
TGGTTAAAAGGTTTACATTATTTCTCTATAATCCAAAAGATGCTCTGTAGATACATTGAT
TGAAAAGAGGTTCATGTCAAGACCCCACAGGATCACTTAGTACTTGGTAGTGTACTGATT
TATTTATTTGGAAATGAAGAGGTAGTTGAGATTTGGCTTGAAATTAAGGGAATCCATTCA
GAGAACCAAACTGGACTTAAATGGTTTTAGGCATTGGCGGCTAAATATATATATATATAT
ATATATATATATATATATATATATATATATTAAGGGAATACGAAAAAAAAAAAAAAAAAT
ATATATATATAACTTTGTGAAATTACAAATAATATGCTCGGGATAAGAACCCGGGAACAT
TTTTATCGCAAGCGATTGAACATAAACCAGTCTCTTTGGTTTGTTGTTTGAACTTCAATT
TGGTACTAGAAAACCGAAATGTGAGTAATTTATGATTGGAACTACAGGAGGAATTTGATA
ATTAAAGAAAATATCTTATAAAATATATATGCATCAACTTTCGGTTCTTTTATGACCCTG
CTCCCCATTCGACCTTTGTATCATCAGCGTTTCTTTGACTTTGTAAAAGGCATGTTCAAG
GTTTTCCAACTGCATATTTTTCACAATTCACAATTCATGAGTCTATCTTGAGGTACTTGG
TTTAATGCTTTAAAAAGGAATTTAAAAGTAAATAAGCATTTCGCTGGTAGTTCCATTACC
TCTTGTCTGGCGAATCTGTTATACACGAATAATCCACTGTACAAATCAAAACTATTTCTC
ATCATAGTCTCCTTCTGCAAGTAACTTTCCATATGTTACATAACCACATATAAAGGAAAA
CTTTTAGAGGAACTGGGGGAAATGTTGACAACTCTTTACCAATTTAACAAGTGTCTCAAA
GCCCTTAGTGTCAATAGGTGTGGATTGAATATCCATTTCTCCCAATGTCCTGATCAAGAA
GCAGCCAAAAGTTGCATCTAGTGAGCATTTACATTAGTGCTTTTCTATTCGTTTATTAGA
TATTAAGGAGGCTGCATACCTGCCAATTGTGTGTGTGATAAATTGGCTCTTAGCAGCCGC
TCTGTCATGTTCCTCACAGGACATCTGTACCATCTTGCAACCCTGCTTAATACAAAAGTA
ACAAAACACAGTGAAATAGATCTACCAGGGATGAGTCATAAATGTAAGGCATTCATAACT
GAATTTTGTGATTCTTTTGTAAACCCAAGCAACAATGGTCTAAACTTATGAAGAATCTAT
TCGAGCTCGCTCCAGGGGAGAATTTAAGATTGAATTGTATACTAAACATTTATTTTCAGT
AATGACCCTGAGGTTCTATAACCTTGGCTGTAGGATCGTGTGGTTAGATTCTTTCAATTT
GGACTTTAACCTAATTTAATCTCACTTGACTTAGGATATGAGATTTGCTCCCCAAGTATA
TACACTACTTTAGTCTTACCTATAGTCGATAATACACCCCTTCACGACTAGTATTATTGG
ATTTGGTAGTAAGATAAGTGATCTGTTGATAAGCCTTGATACAATCTTAGATTTGAATTT
AGGCATAATTTAATCTCATAAACCAACTTTAAGGTAAGGTGATGGTTGTTTCTATTTATA
TACACTATTTTGACCATTTTGTCTCTAATAGATGTGAGACTGATACTTATATTTTTCCCA
ATATAGATATCATATATCTTGATTTATCAAATGCAAACTCAACGTCTAACGTTACTTAGC
TTCCACCCTCTCTGTTTCTTCCGTCTTGATTAACACAATTTACCTGGCCCAATGAAAAAA
ATGTTTCTTTCTTCTTTCGTCATTGACGGGTATTGACAATATTGGACCTGTTTGGATATA
GGCTAAAAGTTCTTTTTGACAACTTCTAAAAATTTCTCTATCCGAAAAAATTCTATATTT
TCAGTAAGAAAATTCTTAAAAACACTTACACCTTATTCAAACAGGCCTAAAGGCATTTAA
CCTACCTCAGTAGCAAAGATTTGGATGAAACTAGAGCAAGTAGCTTCGTCTCTTATCCGA
ACTTTGTCATACATGAAAGTGTGATCTGCCCATCCATTATTGGCAGTCTGAGGACCAAAC
ATTGGGTGCGTGCAGAGTATGTCTGAATCCTCTGGCAACTCTCGTAGTAGAAGCTCTCTT
GGGTGCTCTTTCACAGAAAGAACATCAACAAAGAGCGTTGGTCGCTTCAGGGAAGTGAGT
GGCATTGACCCTACAACCTCGGATAGCGATAATATCGATGTGCACAACACTATAACATCT
ATGTCTGCGGCGAGGAATGCGCTGACATCCCTGTGCAATGTTTGAAATATGGATCATGGA
ATGTCTAAGAATTTGCCTTTAGACATAACTTCACAAGATAGACTTGTTCTTTACTTATAT
ACATTTTTTTTATTATATTACTGGTCGATTGTATGTGATGGATCAAGTATATATGTATTC
ATGCATGGTATGGTTTGGTTTGATTTACCTGAAAAAATGGATCCCCATTTGGAGACAAAG
TTGAGAGTAATCAGATCGAGAAGTTGCTGTGAGAGTGTGGCCTTGTTTAATCATTGTCTT
GGCCAGAAACTGGCCAAAGTTGCCGAATCCAACAATGCCAATTTTGAGGCTTTGGGAAGA
GGATGAGGTTGACATGGTTGTCATAGTTTCAGGAAAACAACAGTATCAGAGGATTTTCGC
TGCTCAATTAGGTTGAAACTTGAAAGTAACGAGGCCTATTTAATGCTGCTCTGTCCATTG
CCATACACGCTTGCGTCAGGTTCAAGGCTTAGGTTACTAGTAATTTAATAGTTTGTCTAT
TCGTTTTTTTTTTTTTATTTGTACTTTAAAGCTCCAACTCAGGAACTTTAAATATAGCAT
CTTAATTCTTATGATTCCTCCTCACACTTTTCTTGTTTTAGAAAAAGAAAAGGGAAAAAA
ATACAATTATCTTCTACGATATTTACATGAACAGATACGGAAGCCTAATAATACTCAGCA
AAATAAATGAACAGATACGGAAGCCTAATAATACTCAGCAAAATAAATGAACAGAAATAA
TTGCCGTAAAATTTTCACTAAAGTATGTATAAAAAATGTACTTTAAAAATAAAAATGGTA
ATTAGAGAAGAAGTAAATACGTTTTGGAAAGAGTAAATTACTACTCCCTCTGTACAAAAT
GATTGAACACAACAGGAATATTTTGATTGTTGAAAGTGTAACGCATTAGTAACTTTTACA
CAATATTTCTTTGGTCTCATTACAATTTGAGAAAAAAAAAATATTATTAAGGCCAGATAA
TAAAATCTATAAATACATTAATAATAATATAAGATTAATTTTATAAAATTGATATTTTTT
TTTCATTTATTTATTATTTTTTTAGTCTCTATAAAATAATTTAAGAAGATAATCATTTTA
GTAGTTCCCAACAATGGTTTCTTAATCTCTAACTTCTTCTAAAAGCTAATCAAAGTGATG
CGTAACTTAAATTTGTTCTTAAAGAGTAATGAACGAAAGGTAACGAGGATAGGACGATAT
TAATTACAAGTTAATTATTAAAACGGGATTACAAGTTTTATATTAATATAAGTTATTTAT
TATGAATATAAATATAATTTATATATTTAAAAATATTTATTTATTATAAATTAATTTTTT
AAATCTATCTATTATATAGTGGGTTAAAACTTAAAATACTATTTTTCTTAAAAAAATAGA
TTGATGGCCACGTAACATTGCGTTAGTGAAGGTGGTACTTTTTAAATTGTGAAGTTGGTT
TAGCGTGTCCCAAAAAAACACTTTTTAATTGTCATTTATTTTAGTTAAGGAAGGTACACG
TACACGTCTCAGACACGACATGTATGGTCGGTCGACAACAAATATTGCTTCAAGACTTTC
TCGGCCTTCTGAACTTTCTTTCGTCAGAATAAAAATTAGAGTCAACACCTAATTTGATTA
GATGCTACAAAAATCACCGGTCTTTGGCTCCTATAATAATTATATTAAATTTTATAATAT
AGCAAAGACATTTAAAAGATTCCTTAAAAAAGATCTGATATTATATTAAATTTTATAGTG
TAATAAAGATATTTAAAAAATTCTCTTAAAAATCCTCTTAAAAATTAATTTATAAGAAGT
GGTCTACCCAACTATATAAGCATTTATCATATATTTATGAATCATCCGATGGGAGACTAT
TCTTAATAAATTATCTTATAACAATAAAAAAAAATCTACAAAAATCACCCCACTTAATTT
ACTCTTTGAACAAAAATATATCAAATAATTTTTTGAATTAAAGTTATAAATGTAAAACAT
TTTGATTCTTTTTTCATTTTAATTTAGATTTTTAAGAATTAATTTGAGTCACTGATTTTT
TTTTAAGAATTAGACTGATGCATATTTTTTTACGACTAATGGTGCACTAAAAAAAAAATA
ATAACAAAGATTAAAAAAATTTAAGATTTTTTTAAACTAAATTAAATACTTACTATAAAA
GAACTAATATAATAAAAAAGTGTTAATTAATGTTCAATAAACATAAGTTAAGAAATTAAA
AGGAAAATCTTTATTAAAGAATATTAGGCGTTGAGGGTGGCGCGACATCAGCTCATGCAT
GATATTTATATGGTTTGTTCTTTCTTTAAGAGAAAAAAAAAATGGTTGTGTACGCCATGC
TGCATGCCACTAACTAAGTTTTGACAGACAGAGCCACTAACTAAGTTGATGATTTAGAAA
GTGCTACTTTGATTAATGCATCCCATGTGACATTGTATGGTTTTCTGCCCAATTTTTTAT
CTAAAAAATTGTGGCATTAAATATTACCGATTTATGTAACAATTTTAGGATAACGAGACA
AAAACTTTCTAATTTGATAAAATATTTTAGAATAACTCCTTTCTAATAATTAATTTGCAG
ATGTGTACTAAACTATTAATTTAATTAAAGTTAAATTATTCATTTGATTCCTTTAGTTTC
ATAATTTTTAGTTTTTTAGTTTCTATAATTTGAAAGTGATTTTTTTAATTCTTATAGTTT
ACATTTTAATTTTCTTTTAGTCTATGTAGGTTTAAAAGCGATTTTTTTAGTTCTTATAGT
TTCATGATTCTTACTTTTTAATCTCTAGAATTTTCAAATTATAGGGACTAAAAAAGAATT
AAAATACAAATTATAAGGACTAAAAAAAGAGAATTAAAATGTAAACTATAGAAACTAAAA
AATCACTAAGAATCATTAAATTATAGGGATGAAATGAGTAATTTAACACTTAATTAATCA
ACTTAATAGAAAATAAGTGTATATAATTTTTAAGGTGAGTTTTAAGATGAGGGGTCCAAA
AAGCTCATGTGAAGTTAAAAATAACTGTTTTTTAAGGAATTTAAAAATAATTGTTTGGTC
ATGATATACAGGAAATGATTCAATTTTAGCCATTTATTTATTTATTTATTTAATAATATA
TGCAATCGTTATTTTTAATCCCACATAGGTGAGGGACATTTTTGGCTCCTCACCTAATAA
TTCACCATTTTTTAAAAAGTTTATTTAACTTAGCACTACACAACACCAAAGTGTAAGCTA
GTTTCCTAAACAACTTGATTGTTTCGGCTCAAAAAGTGTGTTTACGATTCGGTAAGAAAG
AAATAGCTAGATGAAGCAGATAGAAACAAAAAAGAGAGAGAATAAAGTAATTAATAAAAA
TAGGTGAGAAAATAGAGACAGTTTTCATTGATTTTCATTGAAATGTTTTAATACTATATA
TATTACTTATTTTAAGACTTAATAACTTGTATATAAGTTTTGATTTATTAATTTATTTAG
AGACAATTATTTAACTACTAATGTAAAAGGTTATATTATTATGTATTTTAAAGAGACTTT
TAAGTCAGACTTTTGAAAGTAGTCTATATCAACTATATAATTTAAGCAAGAACCTTACTT
CTTAAAAAAAAAATCAAACTATAAAGTCTAACCTAATCTGACCCTCTAATAAATAATAAT
AAAAAAACTGATTAAGTTAGAATGCATAAGAAGAGTGACTTTTAATTAAGTCAGAATTTT
AAAACTAGTCTATATCAACTAAATAAATTAAGCTCGAACATTGAGTTTTTTTAATAAAAA
AAATCAAATTCATCATGTTTTATAAAGTCTAACCTAATCTAACTTAACCAATTTCCATCC
TTCATCTTCACGTAGTTTCGCACTTCCTCAACTTTTTTTTTTGTATACCTTATTATTCTC
ACTAAACCCTATGTGTATATTTTTATATCATTATATAAACTTCCATCTTTTCCATTATTT
AATTAAGGTTTTAGGATTATTAACAACAGTTTTCAAAGTGATTATAATAATTTTCACTTT
AACAATTAGTACAGTAAATATATATATATATATATATATATATATATATATATATATATA
TATATGGAGCGAGTAAATTAAAGTAAGAAATAATTTGGAACTAAAGTGTAATTTTTTTTT
TTTGCCAATATAATTTATTCATTTTTTTTATTTGGCGAATCGGTATTATTCGACGAGAAC
GATTTAGGTTCTCTTCTTGTGTACCCCTCAATTACAGCATGAAATGGTACAAAGTGGAAT
CACATTAAGATTTTTTCTTCTTCTTTTATTTGACATGAATACGCACCATAAATTGACACA
TAAAATAGAGATGCTGCTTCGTGACGCCACTATCTTAGTTTACATAATTTCTTGCTTTAA
TTTACCAAGAAATTCAGGTACTTTCCTTCCAAGTATAGCCTTCCATGATCATTGCTTTAT
CGAAAAATTGGCTTGCTTCAGCAGCTTAGCATCTTCTAAAAGACTTTTTAGTTTACATAT
TAAGGAATCCACCTTGTGAAGACGTGCAGTTTGAATCTTTCTGTGGAAGAGAATTGATAA
ATTATTTTATGATTTGAGATTTGAGATTATTATGTATATATAATTTCTGTAAATTTACAA
AACGTGTAGTTGGATTTTTTAATTTACGAAAAAATTAATATTACGTGTTACAAAGAGATT
AATTGGGATTGTAAACATTTGATGATTCTGGATCATTTTTTTTTTCTGATTAGACTTGTA
GCTTAAGGATTTTATCTATATTTTCTTTTTTCTCCTTGTTTTGAGTTTTATTTTAGTAGA
ATTAAGGGGATTTCCTCGTTACATGCTCGTAAGGTTGAATACCAAGCAAAGTTGGTTACG
CGTGTATTTTCCAAAATGAAATCACATTTTCCCATATCTATGCCTCTTCGAACCTTTAAG
GAAGTTATTAGTACCATTTCCGTCTTTTTTGTAATTCAAAATTATTAAGTGAGGACAACG
TATTAGTATTAATCGTGAAATTTGTGTTTGTTTTCATTAATAAATTATTAAATTGTAAAA
TTGTATATATTTTTTGAAAAAAAAAATATTTTTAAGATAGTAATTTTTTTTAATGAATTG
TTCTATTAAGAAATAAATGTTTAAAAGTGAGACATAAATTTTTTATTATCAGTCTTTTTT
AGTCAAACAAATAAATTTTTAGTTTTTAATTTAATGGAGAATTATTTAATATTTAATGTA
AATTACGATAATAATTTAGATAAATATATTCATTTTAGTTATAATAAGGTTATAATTCAT
AAGATAATAGAATAACTTTTGATAAAAAGTATTTTCAATTTAGTAATTAATTTGTAAGTA
ATTTTGACAATAATAAAAAATAAGGATTAAAAAAGTAAAATTTTGAGAAGAAATAAAAAT
TAAAAAAAGTTAAGGGTGTAGAAATGTACAGTAAAACTGACATGAATTCATAAAATTAGA
AAACTGAATACATACATGATAATATATTGAACATGTGCTAATTTTGATGTAGGCGTGTTG
ATGTTTTGAGAAATTAAATAATTGTGTATCAATTCTCTTTATTACAACATACAAGTTCTA
TATTTATAAAGTATAAATTATAACTACTCATAACCGCTATAAATTTCCATTAAATCTTCA
CTAGCAATGACTTTAATCTTTGATCTCTACCATTGATGACTTTGTCTTCAATTTTTACCG
TTGATGACTTGGGTATTCGATCTTTACCCTTGATAACTTGTATCTTCAATCTCCATCGTT
GATATCTTCCAGTGTCTTGATGCTTTTTAAATCTTCATTTCTTCTATCACCACACAATTA
GCCAAAAGATTATATTTTGACACTTAGTCTAAATAGTCTATTAAAAGACTATTTCTCAAT
CTTTCGACGAAGATAATTTTTCAGTAATAAAAAAAATCTCAATGTTAACACAAGCATAAT
CTCATAGGTATATTCAAGGAAGGAGACAATTCTTCCTCTAAAGGCCTTTTATCCTCCTGG
TAGGCTCTTCGTCATGAGTTAATGTTAAATTTTTATCAATATCAAAGATGGATCGTTGAA
ACAAATTATAAATAAAAGAGCGACGAGGTTCAAAAAGAAATAGATATTGAAAAAAAGTAA
AGTTTCGAGAAATAACAAAGAATGAGAAAAAAGTAAAGGGTTTAGAAAAAAATAAGAATT
GGAATTTAAATATTGGTTGAGAATATTGTTCTAACATGTCAATCCATAAATGTTGCGTAA
ACTTATGAGCGTCGCGTGAACTTAAAAAGATACGAGGATGATACATGGTTCAACATTTAT
TACAATGATACAAATTGGTATTTACAGACAATAATTCATAATTACTCATAACTTCTCAAA
TACCTTATAGTAACATCATCTTCACCATTAATGATTTTTGTTTTTGATCTTCACCCTTGA
TAACTTGTATCTTAAATCTTTATTGTTGATATCTTTTATTGTCTTGTTGTTTTTCAAATC
TTCATTTTTTCTATCACCATATAATTTGTCAAAAAATTATATTTTGACACTTACTCAAAA
TAGTTTATTAAAAAAATAATTTTTAAATATTTTATCGAAGAATATTTTTGGTATGTTAAT
AATTAGTATTAAGTATCATCCATATGTGATCAATTTTCTTTTTTCATTGCTTTGTCGAAA
GTAACATTGTGGATTTAATATATCTACACTCTGTCATACTTGTCCACTGTATCATTTGGA
ATATCATATACGAGCTGATCTCTGTGATTAGGCACTGGATTCGCATTGACATTGTGTTTA
ACAAGCGTCCCTCTTATTCTCTGTTGTTGCACAACCAACAACGATGCCTTGGTCACAAAA
TCTGGTGATGCTAGCATGTCCCCAATTACCCCTAGTTCTTGGCACTCACTCCATTGATCA
TGTCCCTGAAAAATAACACTGTCTGGATGCTCCCTTCCCACCATCACTAGATCAAAATAA
TCTATCAATCTTCTAATACATGTTGACATCTCTATCCCATTTTTCACCACTTCATTCATA
AGCTCAAACCTTTGATTTCCCGCATTGTAGTATCTATACTCATCTATAAGATCACTGTCA
CGTTTTCTATCTTTAGAATTTTCATGTCCAAATAAAAGGAACCTTACCACAGTTACATAC
ACACATTCATGCCTAACCATTCTGGAAGCATAGGCTAATGTCTCGGCGTCATCTTGGCCG
CCGATGAAGAACACAGCGACATAGAATGCTGCTCTAGCCATCAAGAGTGAAGGAGAAGGA
CTCAAGATGCCCCTATCAACTAGAATTCCAACAGAACATGGTGCCCTTTCGAGGACTTCA
ATGTTCATGGTTTGGATGGTCCTATGAGAAATCTCAACGGTGGCATCAATTTCCCACCTC
TTGTGGAATGGCAAGATCAAAATGTTGGATCCAGTGTCTAGTGAGATTTTACAAATATCA
TCATACATGGTTTCAAAGGTGGAGATTGAAGTGAAGGATTGAACAGATACATATCCTTCG
TTCTGTTGTGCGTATTGCCTCAATGCATTGTCAATGTGACTTGCATTGCAAGACATTGAG
CGCATCTCATCATGGGGTTGTTCTTGATTAGCAAAAAGGATAGGCCTGGCTCTTCCTTGA
AGCTCCACTAGGACTAATGCTGTTACCTCAATCCTGCTCTCTCTGCTTGCATAGGATGCT
TCTAAGAGGTTCAGGATCATGGGGAGATTTTCGTTGTTGTGAATGCACACCATCACTCGG
AGCTCCAAATCTCGCCTAGTATGCTGAATTGTGCATCTTCTTCCGGTCTGATATAGTTCT
GAAGGATCATATATGTATCTTATGAGAGGTACTAGGATACAATTTACTACTATAATCGAG
GCCACCATCAAAGCGAACTCTTGTTCCGATATTAACTGCCATAGACAACAAGTTTTAGAC
TACGTTAGTTAGTTATTTAGTTAATCAACCAAGGGTAAAATGTCCCTCAATTAACATTGG
TTTTAATTTATTAAATTTATAGTGCAGCATAGGCATTTGAAAGGTCCCTTTAAAAACCAG
TTTATAAGGGGATGGCCTACCCAGCTATATAAGCACTCATCATGTTTGTTAAACATCCGA
TGTGAGATTATTCTCGACATAATCCATATCCATACACTTTAAAAATGATAATTTTGGTTT
TTCGTATTGAAATTAAGTTCACTATTCTCGATCTCATATGCACATGCATACACTAAAAGG
GTCTAACTCAGTTAATTGATCTGAGTGTATAAGTTATTGTAAAACTCCTAACACTATCTT
TAATTTCCATGGATAAAAGAAAAAGATGCACATACACACCTTGCCTCTTTTCCACATATT
GTAAGTGGCAAGCTCAGCTATACCCCTTCCATTTAGAAAGAGGCCAATGACAAAACATTG
TTTCAAGGGTAGATTGTAATAGTATCCTGGCAACATGACAGCACCAATCTTTACAAAGAA
AGCGACAATCAGAATGACACACACAATCCACAGAGACTGCTTATTAATTTTGAAGAAATC
AGTTTGCAGTCCATTAACAGCGAGAAAGATTGGATAAAGAAATGCCGTACAGATTGTCTC
CAATTTACTCATCAAAGCTGTTCCTATGGGTGGCCCTTCTGGAACAGCCAAGCCTAAAAG
TATAGGTCCCATTATAAAATGCTGCCCAATTAACTCACTGACAAATGCAGATAAGAGAAC
TAAGAGAAAGATGCAAACAAGGCAGATTTCATTAACCGAACCTCGACCTGGGTGCTTTAC
CATCCAAATTATTGTTGGCCTCATTACAAATATAACAAGAAGCCAGACTCCAACTATGGA
CAGAAGGATGCATGCTAGTCTCACAAAGCTACCACTCTGATCCTGCAGTATTGCAAACAG
AATCACTGTCAAGATAAAGCCAGCTACATCAGCAAACATTGCTGCTGACATCGTTAAGCG
TCCTATATCAGTGTTAAGGACTTTGAGATCGGTCAAGAGCACTGCTATGCTAATGAACAC
AGTTAAGGTTTGCGACATAGCTAGAAATGGCAGTGCCTTTGCGAGGCTCCCGTCCATCGC
AATGTATTTCATCATCAGGAAGGCCAGTCCAGTAGGGATTATCAATGTAAATGCGAATAC
AGAAATACCAAGAGTGATGGCCACCTTTTCAGTCTTCATTAATGTGGCAATATCCATTTT
AACACACCATATGAAGAAGAAGAACATAAGACCAAACAATGAAACTGTGTCAAGCACTAC
AGCGCCCTTCATAGGAAATAAGGCTAGCCCCAGAATCTTTTTGTTCCCCAACATTGATGG
CCCAAACAGCACTCCACCCTGCAATGTTACCATGCATCATTTCTTAACACTGGTTCTTGT
AGCAAGCAATGAACTTTATTTTCAATTGATACACGAATAACATTTTTTTCCCATTATTGG
TGGTAGGAGTGGCAGCAACACACACTATCACAATAATTATTACATGGTCCTGTCAATCTT
CATGTGCCTTTATTGTAAATTGGAAATTTCGATTCAGTGTCTTGTGAAGATGGACTGAAC
CATGGTCCCATGGTGACCGAGGACCATGTAATATTTTCTCTTCTAACACACTTTTTATTA
TTGGTTAAATTTATTAGATACTACAAAAGCATGAGTCAAGCTCATTGAATAATATGCATG
CAATTTTTTGTTGCTTTTGTAAGTGTCATTAATCACTATATTTAGAATTAGATGGTTATA
GAGCATTGATTATAGTGACCATCATTGATTAAACACCTTGTAAAATTTATGAGCTTAAGA
GAAAGCTTACCATAATCTGGGATACAATGGAGGATTGACCTAGTGACTTGAGACATGCGT
CCATAAACTGTGTGACCATGGTCATCAAAGAGACCTGCATATAGAGAAGACAACTGGGGG
ATTCTAATGGGTTGCCTCCTTTCCAGATATCAAGGTGACCAAAACTGTGGGGATCTTGGC
ATACCATTGACTTGTTTCCAATTTCAGATGCAGCGGCCATATTTGTGAACCAATATCAAG
GGAGAAAAAACAAACCTTAGTTGTTATTGTAGTTATTGTTAAATAAAAGTACTTTTCCTT
GAGAAAAAAAAAAAAAACAAGTATACACGAGAGAATAAGAGGTTGACCTTGTATTTTGCA
TTTTACATATTTAACGGTTTGCCTTCAAGTAAGGTGGTTATAGCAGCCGTTTACTCTTGC
AAAGTCATGGAATATATATATCTATAATTATACAACTTGATTTTGGAGGATACAAATGAT
ATTAACTAGAGACCTTTTAGCAAATCCACAGAACTATGTAAAATGAAAAAGCTATTGCAC
ATTTGCCATAGAAATGAATCGTGATTATTAGTTAATATTGTCAATTTACATTGCTTAGAA
ATCTAGAGTCCACGCTTACTTGAGCTAGATTATCTGAAAAGGAAATTAAGGGACGTGTAA
GTGTAACCTTGTTTGCAGCATGGTCATAGATGGATCCAACGTCTCAAATCCAACATTTCT
AGATAGCAATCAGAGACTGCGACTTTTACCTTCTTAATATCATCCATTTTCATTTTCGTG
TGGTGCAGATCATGTTACTTTTTGCTTCTTTCATTTTTATCCCCACTTGTATGCATAATA
ATCCGAATATAACATTAAATAAATTTTAATTTTCATGTGCTGTTAATTATTTATATTATC
AATTAATTAAAAATTAGTTGATAAGTAGAACTCTAAATTAAATTTCTCAAATAAAAGACA
AGACTTAATTATATGCAGTTTAGTGTTGTTTTTCATTAAAATTAAAATAGAGATTTCATG
TCAGTAATTTACATAATAAAAGCTCACATTAAAAGTAAGCAGTGAAACTTCAGAACAGCT
AAACAACGGCATAATCAATCATCCAATGGTTCAAATATATATATATATCACAATATATAG
TTCAACTATTCACAATATATAGATTATAGTATACAAGGATAGGTTTCGAGGGGAGAAAAA
GTGTACTATTCTAAACAAGTTCACTTATATAAGGGGGTTAATTCAGGAACTTGAGCAGGG
TAGGTGTGCGAGTGTTGTAAAACGTGTTGTAAAACATCTGATACAGTGTTCAATTCCCCT
ACGGACAAAAAAAAAATTCACTTATTGGTTTGAGCATCTTCCCAGGCAGTTGCATTCTTG
CCAATCTTTTGAAGGACTCTGATCACTTGTTCTGTTGTGACGTTTCCCGTAACCATGACC
TTGTTCAGCTGTGTGTCCACGTTATACGTTTCAATATCTGCAAAGAACACAAGTTTTAAA
CTACTTAACACAGTCAAGGAGTCTAATTCAACTTATTGAACAAGGTGAATTATTGTAAGC
TGTTGACACTATCTTCAATACCCAGTTACCACAATAAAAAGAAAAAACTAATTAACACAA
AAATATGATTGTGTTTTTGGTGATGGGAAAATTAAGAACCTTCAATTTTCTTGATGGCCT
TGAGGATTTTCTTGATACAATCTTCACAGTGCAAACCGACTTTCACTTCCACAACCTGAT
GGTAACATGCAACAAGAAAAACCTTCTCAGTCTCATAGTTGCAAACAGAAAACATGAAGT
TAGTAAAAGCCTTACATTTGCCATGAGGAGAAGCAGAAGAGGTGTATGTAAAACTCCAAG
GCTTGGACAAAGATGAGAAGTGGAAGAGAAAGAGACAAAAGAAAAAAAGAGAAGTGGGGT
TCTAATGATTTATGGAACCGCGTGAAATGGAGCAAATACAAGATGGAATTGGATTCTTAA
CGTGAATTATGGAAGCTTTTACTTTCTGTAAGAAATATTTCGAACAAAAGGTGCAACTTT
ATTCCTATTTTAAGGTTTGGGACTTGGGAGTGTCTGGATTCCCCTAATTAGCAATTGAGG
TCGGCAAGCATCATGCTTCGCCTCAAAACAGGATAGGAATATGTAATGAAACAATAAGGA
AATCTTTATCTTCAATTATCATAAAATCCAGCGTGATTACTATTACATTATTATTATGCG
TGTATGCACCAGCATCTTTAATTCATAATTGATTAAATAAGTTATTTATCTTAATTTTAT
GATATTTTAATTTCAAGAGTAGCTTTAAGTATTTAATATAAAAAATATTTTAATGTTAAA
ATTGTTAAAATAAGTGTTTAATTTAATTTTACGGGTCTTATAATACTTAAAAATATATAT
TAATTTTAAACAACTCATGTAAACATCTTCAATAGAGATGTTTTAAGTGTGTGCATAGCT
AAAACGCACAACTCTATTCAATGATTTTAATATTTTGTAATTGTGAAAATCTTTCATTAA
TTTTTTTTAAGTTTGCTTTTGTACTATTTTATTTAGTTATTAATTGAGTTTCACATAATA
GATTATAAAATTTATCAGAACAGTAAAATGATCTTTTTATTTGTTTTATCTTAAATGAAT
GAATTTTAAGCATAATTTTTTAGTTATTTTCTTTATTGATGGGGTTAGTTTAGTTTAGGA
AAAAATTATGCCCTTTAGTTTATTAATCATTTGAATTAGATAAGTTTCCTTTTTGGGTTT
ATATATTATATATACCGTTACTATAAACATTTGTAATATTGACTATTTTCATGAAGACAA
GTTTTCTTCATTTATCTGATATTTGAGTTAGCGACACCCATTCACAAAAATTACCCAAGT
CAATTATTAATATGAATTCTTCTACAGTGTCATGTCCACATGTGTGATATTTATTACTTA
CAAAGCACCCTTTACCTAATAAGAGATAAAAAAGCAAATTAAGAATTTAGGAAAAAAGGC
AATAGCACAACGTAAAATGCCATCACAGCCTAGACTTCTCTTAATTCCATTAGTTGGTCC
TGATTGTCAACAACCATGATAGCATGCAAGCTTTCTATTTGTAAAACCTAAACCAGAAAG
TGATTATGCATTTGTTATCAAGAGCAATTATATGTCACAGTTTTTATGGTAACTAAATAC
AGTGTGATCCTATTAATTGACTCAAAGACACAAGACACAACACTGAATGCACACTATCAA
TTGTACTTGGCAACTTAATATACTATATACTGATTGCAGCAACACCCAAATACACTAGAT
AATAGTACGGGCTTGTTTGGTTAAAAATCTTAATTAAGCACTTATTAATTAAGTGTTTAT
CATACAAGCACTTTTGTACAAGTTATTTCTAAAATTGAAGAGAAAATAATGCTGAATTGT
TTTAATATAAGTTGTACGGTATTTTCATAAGCTATTCAGGACTTACAGAAATAAGTTAAA
AGCAACTTATGAACTGATCATAAACTAATTTTATAAGCTTTCACAAACATTTACACAAGA
AATTATATCATGAGATAATTCCAAATAAGTTGTAAATAAGTTCATCCAAATGCATCCAAA
GTGTTAATTGATTTTGACACGAGATACACAAAATAGATGTCAGTGCCCAGCTGCCTGACC
TTTTAAGCCTTCAGATTAATGGAATCATATCACACTAAATTTAAGGTTAAATAGTTCAAG
TATTATTCGAGTTTGATTCTTAACGAAAATAATTTTTGAACAGACTTTATTTATCTTTCG
ACTAAACTTTGAATTAATCAGTCTCTTTTTCCTAATGAATCAGAGGATTAAGACAAAAAA
AATATCTAAGATACTGGTAATACTTCTAGACTGCTGCTAGTTAGTGAATGTTTAGCAAAT
GGTTATTTTTGGGAAATGTTCGCTTTTCTTTTATAAAAACTCTCTTAGGAAGCTGATAAA
AAAAAGTGATTATTTTTTCAAAATGAACTTAATCGAACACATTTGGTTTCAGGCAGCTTA
TCATACTAATTGCAGTGATGCCAAAGTAAACTAGATGTTAGTTAGTAACTATTGAACTAC
AAGTGTGAGGTGAAAACTCATATTATGTAGGAATAGGAAGGTTGAACACCATATAAGTGA
AGGAAAGACCCATAAACTTGAGTTTTAAGGTTTTGGGTTAAAGTGTGATATCAAGTTCAC
TTACATGATTACTCATGCCTCATTGGTATAAATCTCCTCGTTGTTTATCCTCCTCGATTG
CACAAAATTGATATCGATTTAGGCAGTGCAAGTAACTAGAGCAAGAGGGGAAGGGTTATC
ATGTGGAAGTAACTCACATCTGAGGGAAAGATGGCTGGAAAAGATTGCCGGAATATAAGT
GTGATGTTAAATCAGACATCAGACTATTAGAAAATAACATCTGTAGCTATGATCTCACAT
AGAATAGAGGAGAATGGATTAAAGCCTCTGTGCAAGAGTTACTCTCGAAGTCCTAGATGC
AGAGTGGTCAATGCTAGGAGCATATCAATTTGAAGTTAAAGGGGGAAAGGAAGTACAGAA
CACAATACTTACCTAGGCATAATAAATAAATCATACAATAAGGCACACAGTTGATGTTTT
TGTTAGAAAATAAAACCAAAATGAAGGAAAATAAATATGAAGAAGATGCATAGTCTTGAA
TTAAATAACAAAATAACAATAACAAATTAAATTTAATTGATAGCACATAAATAATGCATT
ATATCATACAATGAGCACAAGGAAAAATTAAATCAAGGGAAAGAATTAAATAGCCTGGGT
TGAAGCGCGTAAACGCTATCCTTAGAGAGAAAACGCCCCCACTGCACGGGTAAGAAATTC
TGATTGCGCTCCTCTCCCAAGATACGACAACCCGTTGGTTCCGATCGTGTGCAGCGAAAG
GATCCCCGAACCTTATGAACACCAATGCTCTTGCTCTCACAAAAATTAAGTTTTGTATAG
GAAAAGAAAGAGATAAATTTTTGGCAGAAAGAAACCAGAGCTCTCAGTCTGTCATTTCTA
GAAAAGAGGAGATAGATATATATAGGCATTTTGCAACAACAAAATATGACCGTTAGAAAT
ATGACCGTTGGAAAACCAACATACAGTTGTCACAACAAATATAACAAACTAGTTTGACTC
ATTAAAACAAAATCTTAAGAGAATCTAAAATAAATATTATTTTATAAAATAGAATTAATA
TATATTTATAAATTTTAAATTAAAACACAAATATTTATCAACATTCCCCCACATAATTTA
AAATTTTAAAGTATATTTTCTAAAAGATAATTTGTATAAAAACATAAGAGAAAGAGCATG
TGATATTGTATTTCGGTATAAGGAACCTTCCGGGTTTGAGCCTTATACCTAGTGTTTATG
AACTTCCATCCGAGAAAAATGTAGTGACTTGATTGTCTTGAACTACATATTCTTTAACCG
GACTTTAGTACACAACCCCTACAATATTGGTGTTCAATTAGGTTCTAATCAGTGGTAGCG
CGTTACACGGCCTTGCGCTTGTATCTTGTTTCGTGAGTGTCACTTAGAGATTAGCCCATA
TCTCACAGTGGCGGCCCCACCACCACACTCACTAGGTGAATCCTCAAAGAGTGAGTTGTG
ACCCTCACCCCTACAATAATTGTCATCGGATTCATTAAGAGGTATTTTGTTTTTTTTTTA
TACCAAAAATACACATATATAAATACCTCAACCTTAATATTGCCACAATTTATAATACAC
CTCGTCATAGGAATGAGAAACGGAACAACTCCGCCAAATTTCATTTTGGTGTACTTTAGT
CAACAAACAATTTCGTTGTTACCCGTTGAACTCCATTCATGGGATCACCAATCACACGGA
GACGGGTGTCCATTGTTGTAACTAAATAATGGACTTTAATCTCATTCCCCTCGACGACTC
AAATACTTGTTGACATGTCAACCTTTTTGTAAGCGGATCCGCAATATTATTTTTTGACCT
GACAAAGTCAAGAGAAATGACATCATGAGAAATCAAATTTCTTATAGACTTATGTCTCAC
TCTTAAGTGTCTTCTTTTTTCATTAAAATTTTGCTAGTCCCTTTAGATATAGCAATTTGA
TTATCACAATGCATTGGAATTGGAGGTATAGGCTTATTCAACAATGGTAGATCACATGAT
ACTGCACCACTAGTAGCAGTATCTAAATCAATAATTTCTACTTCCATAGTAGAACGTGAA
ATAATAGTTTATTTAGCAGATTTCCATGATACTCACAACCAGCTAAAGCAAAAACATAAC
CACTTGTCAATTTTATTTCATCAAAATTAGAACTTCAAGTTTGTATCACTAAATCTCTCA
ATTATTTTCCAATCTACCAATTGCATGTGCAATATCAGACAGGCCTAGAAAAGTTTATCA
AATGCAACAAAGAACCGATACTTTGAGAATATTTATGTGAAGAAATTCCTTTACTCAAAT
TTTTCTTTAACTTGATGGATGAGTCATAAGGAGTAAAAACATGTTTCGCATCAAAATAAT
TAAACTTCTTCAATAGCTTTTCAACATAATAAGATTGGGTAAAAAATCATACCATCATTT
TTCTCTATAAGCTTAATACCCAAAATCACATCTACACAACCAAGGTCTTTCATATCAAAA
TTTCTAGACAATAAAAACTTCACATCATTTATGAAATTCATTTACTACCAACTATCAATA
TGTCATCTAGATATATAAAATGACGCATCCATTATCATCAAATTGTTTCACATACACACA
TTTATCACTAACATTAATTTGAAAATCATACAAAAGAATAACTTAATCAAACTTTTGTGT
CAATGCTTTGGAGCTTGTTTCAAACCATACAAAGATTTAACAATTTATTTTTCAAGAAAA
AATATTTTCAAAGAAAGTCACATCTCTAGACTCCATAATAGTACCATTAGAAATTTCAGA
TACTTCTGAATTAATAACTAAGAATCTATAAGTAGTATTATGTAAAAAATATCCAACAAA
ATATAATTAATATTTTTTTTAATTTTCCTTTTCTTATTAATAGGGATATTAACCTTTACT
AGACACCCCCACACTTTAAGATATTTCGGATTTGGTTCTCTTTTTCTCCATAGCTCATAA
AGATTTTTCTTTTGTTTATAAGGTACTCTTTTAAGAATACGACATGCAAAATATAAAGTT
TCACCAAAGTGTTTATTTTATTATTTTTGTGTGTTATATTTTCACAGGCTTATCTTTTAT
CAATGAGTTATAAATAAAGAGACAATCAGTCAACATGTAACAACAAAATACTTGCAGTAG
AAATAATAACATTAAACAATAAAAATTAAAAACCAAACAACAAATGTCCTAATTTTAAAG
ACTTGTGTTCACAGGATCATTTGACCAAGTAAAAGATAGTTTTCTAATCATATAGGAATG
AAATTAGAAGTATGCTTTTAGTTTTTCACATAAACTAATTCTAAAAGCATTTTCTCTTCA
AAACCATCATTAATGAAGAAAATATATTTTAAATTTCAAATTATAAGAAAATAATTTTCA
ACAATCTCTCACTTATGTAAAATTTAAGGAAATGAAATAATATAATAAAACATTTTTAAT
AAAACATAATACATTGTGTCTTCATCCATTAATTTTCGAGACTTACTAAAAAGGGAGTCA
ATCATATTCATGACAGATATTTTGGTAAAATAGAATGCTACTGTAAAAAAGAGACTATGC
AAGAAGAAAGTGATAACGATTTTCTCTCTAAGACTGTTGGAAAATAAAACCAAAATGAAG
GAAAATAAATATGAAGAAGATGCATAGTCTTGAATTAAATAACAAAATAACAATAACAAA
TTAAATTTAATTGATAGCACATAAATAATGCATTATATCATACAATGAGCACAAGGAAAA
ATTAAATCAAGGGAAAGAATTAAATAGCCTGGGTTGAAGCGCGTAAACGCTATCCTTAGA
GAGAAAACGCCCCCACTGCACGGGTAAGAAATTCTGATTGCGCTCCTCTCCCAAGATACG
ACAACCCGTTGGTTCCGATCGTGTGCAGCGAAAGGATCCCCGAACCTTATGAACACCAAT
GCTCTTGCTCTCACAAAAATTAAGTTTTGTATAGGAAAAGAAAGAGATAAATTTTTGGCA
GAAAGAAACCAGAGCTCTCAGTCTGTCATTTCTAGAAAAGAGGAGATAGATATATATAGG
CATTTTGCAACAACAAAATATGACCGTTAGAAATATGACCGTTGAAAAACCAACATACAG
TTGTCACAACAAATATAACAAACTAGTTTGACTCATTAAAACAAAATCTTAAGAGAATCT
AAAATAAATATTATTTTATAAAATAGAATTAATATATATTTATAAATTTTAAATTAAAAC
ACAAATATTTATCAACAGTTTTAAGTTATTATATCATAACATCTTCCTGAACAATGAACA
ACACAATAACAACTAATTCAAAAATAGAGACATCATTGTTGAACAATTGTCAGTTAACAA
AATTTGACTTATGCTTTGGATGTTACAATAAGTTCACATATACTATACTCTAGAATTGAA
ATTGAAATTTAATCTCTTTGAAGAAACACGAGTTATACTTGCAAATTAAGGAAAAGATTC
ATCTTAGTTCTCCAATTCATGAATCAGAATCGCTTGCTCTGTATCCTGAATGTCACAAAA
TAGAGATCAATCAGGACACATAAAACCGCAATTCTTTAGTCAAACAAAAGGTAGAGCTTA
CCTGGCCGTTTACCGCACACTTTCCAAACTGAATTTCGATTTCTTGATGCTTTACTAATC
CAAACATGCCCCTATAAACAAAGTTTGTTACAGTATAGACAGTAACCTGAAGTTGAGAAG
CAGCTAATAGCATACCAATTACATAATTCAAATTAACTGCAGTCATGGGTCTTGATGCCC
TAAGAGTCCCATCCACCAATGCAATCTAAGGTTTTGTTTGGATAAACTTCTCCATAAGCA
TTTATAGGAATAAGGAAAAATGAAATAAGTTTCTTCCATAATACAAAAGAACTTCTACAA
GTTAGCAAATTAGCTCATGTATAAAAGAAGCTTATTTCATTTGCCTATGAAGAAATTTAT
CCACACAAGTCCTAAGGAAAAATAGAATCACAATTAAAAATTCAAAACAGGCAATGGAAT
TCGCCCATATATTTTTTTTTAAGTAAATATCAACAATCTGATAAAAGTTATAATATATAT
GAGTCTATTTTTTTTATATCGGCACACGTTCGTTTGTTAGTCTTGTTAGAATTATCATTC
GAACCCACGACCTCTTTTTCTCCCTTCTCCTTTAACCATTCCTCAATCACTAAACCAAAC
TTATATCTTCACATGAGTCTATGACCATACCCTTTAGAAGGGGTGTGCAATCAATTATCA
ATTATAATATTATACATAGATTGAAAAAAAAGAAGAAGAAAGGAAAGACCTTATGAGGAT
CCCTTGGAACACCATATCCAGAGTAATACATTTGGCCAACCAAAACCTGCATGGCAGAGT
CCCCAGCCTTGGCCTCCTTGAGGGTGTCCTGGAACCAGCGCTTGGAGCAATCTGCGACAA
CCTTGGCGAGGGGTGTCCGACATTCCAAAGGTTCCATCTTTTTCGCAGGTTCGACCTTAA
CACCAATTGGGTTCGCAATTCGAGGAGTGGCAACACGGTTGGGCGATCTGGGTAGCGTTC
CCTTTGACCTATTGGGCTTCGCAGCAGATGACACAATCCGCGATAGCTCTTCCAACCTCG
CCGCAGATTCAAGATATTTTCCCATCAAATCAAACCTCACACACAACAAATAAAAGTGTG
GCTTTTAAGTTCAGATATTTCAGGGCTTTTTTATTTTTAAGTGGACGATGTTGTTGGCGA
AGACAAAGATCACTATCCTCAGACCTCAATCTAGCTTGAAAGTCGAAATGGTTAGAGTTA
GTTAGACCTTAGAACAAAGGTGTGTGTAATTGTGCATGGTATGTGTTAAGTGTTACCTGT
GGTAAGAGGAAGATGATCAATTGATACAAACAACACACTTAGGTCAAAAATTGAAAGGCC
TAGGATAATTGTTGGCAATGACAAAGATTGCAAATCTACCAATAAAATGGTGTATTTTTT
TTTTTCAAGAAAAGTAAAAGGTGTATGCAGTCACAGGTAGTTACACTATTATTTAAATAA
GTTATTTTCATAATAATTACTTACGGTTATATCTTATTATACTTTTTATTAATTGTTGAT
GTAACTTTTTTATATATATTAACGACGTATGAAAATTAATTTTATTTTCTATATTTTTTT
ACAGAATTTATTTTTTCTTCCTACTTTATCTAAATATTTGCTTTATAAAATGTTGCGCTA
TTTAAAAGGTAGATAAACATTATTAAAATCTACTGGGTTATAAAAAATAGGAAAAATAAA
TAATATTTTTAATGTAAATATCATAAATATAATACATAAAAATAATTAATGAAAAATTTT
AAACATAAATATATTCATTTATTGAATTATCATTCAAATCATCACTAATCAAAATTATTA
AATTAAGTAATTAATCATATATAAAAATTGATTTGATTCGAACAAAGTCATTAACTTTTA
TAATTTATTTAAAAAGTTAGAAAACAAAAGGTTAAGAAATATGTACATGAAAAATAAAAG
TAATTAAATAAAGTAATAAAACAAAAATAATTCTAAAACTGTTGTGATACTTTTAGGGAA
AATATTTCCATACTACATATTTCACATTTTTCTTCCTCTTAAAAAAATACGTTTGGACCG
CAGCACCCTCTATAGAAAAATCAATGCATATTCATATATTTTTAATCATTATAAATAAGA
TTATAATTCTATAATGAAACTTAAAATATCTTTACAATCTTTGGTCAGTTAGATTGATTA
TGGGACTAATTAATAGTCCAAAAAATCACATTTAAAATCTTTTTTTTTCTTTTTAAACAT
GTATTCTACAAAAATTAAACATAGATTATTCTTCGTAAATTTGGCATATGTTATCAAGTA
AATTATACAATACATATTCGTATCAATAACAATACAAGTCTCCTGTTTCGATCAAGCATC
TTTTCCATGTTCAATTAAAAGTCTTTTTCTAGTTCAGCCATTATCCTTTGATATCCTTTG
GAGTTATGAGTTCCTGAATTACCAGTGCTACCATTCATTCCTTCGTCATGTAGCTCAGAA
TTATTTTTTGACCATCTAACAAACCATTTGGTATAATAATATGCAGATATGCGCTTGGTT
GGCCTATCTATTTTTTTACATAGATTCCTTCTCTATTTTGGTTTATCAGTAGCTGAATAC
AAAGATTCCTTCTCTATTTTAACAATACAAGTCAAGCTTGACTTTTTACAATTTGATTTT
TTTTATAAATTTAATAAGGGTTAAGAATCTTGACTTCTGTACTTTGATTAATGGATTTAT
TTACGGTTCATGTGTCTGCGTTTTGATTCATTTTAAGAATCTTGTCCTTCGTACTTTTGA
TTTTGATAAGTCATTCCAAAAGGAATCCTTTACGTTTACGTTTGTTTTTTCGGGAATGCA
ATTTGAAATGTCAATCATCATGAGATGAGATTTTACGTAGAAATTGCTGTGTATGAAACT
TGGTTTATAAAATGGTGCTTGGGCTCTGATTTTTTTCACATGCTCTCGGCCATTTCACCC
CATCTTCCACTCTCGGTAGAAAGATATTTGACAGCGTGAGAGATATTTTTCCTGCAAAAC
AAACCTGGAAAATGAAAGTGAGCGTTGTTGATAATTGATAATTGTTGCATACGTCCATTT
CAATTATAAATAACACAGTAATTATTTATTTTTTTCTAAAGTTTTATTTTAGTTTAGTCT
CAATTTAATTAGAAAATGTAATTTTATAGAATCTTAGGCTGTAAATAAAATTATAATTCA
TTTCTCCTAATTTTTACTTTTCTTTTCAAAGATGTTAGAACATCAATTGAAGATGTCATA
CACAATGTTTGGCCCAACGAGTTATGTTGGTCCAACCAGATTAATTGTGCAAACAAAATC
ACATAGCAATTCAAGAGTAAAGAAAAATGTCAAGTGATCAGGAGAACTTGTCCAATGAGA
AAAAATTCAACTAGTGAGTAAAGATCACAAAGTGAAGAAAAAATATTCAAAATATCAATT
ACAAAGAATTTGCCTAGCCACTGGAATCACATTTGAAGTGTCTTTTATAATTCTTAAAAA
AACTTGCTCATTGAGTAGATTATACTTTGAAGTGACAAGATAAAGATTTTGAATCCCATA
TAAAAGGACATTATCTTAGGATTCCCAAGGGAGGCATAAACCTAGAATTGTGTGTGGTGA
ATGGAACTCTCATCTACTTTCATCTTCCATTTTCATCCATTCCCCTTCATTTTCTCCTTT
CCATCTTGTAATTTTGAGGCTTCTCATGACAATGAGATACTAGTTCATCAATTATTGGGG
GATTGGTATATTAAGTTACTCCCATGTAATTAATCTTTATATTTATTCAGTGTCATTTCC
TATGTTATTGCTTTTCTACCATTTTGATTGCTTGTTTTATGGTTTGATTATCTTAGCTTC
AATTCTTTATTTTTATTGTTACTGGAAAATGCTTGAAAATTGGGTTTTGAACAAAACATC
TAATGAATGTCTTATCTAGGGATGGAGGGATGCTTGTTAGTATCATTACTAATCAACCAT
ATGAAAATGTTTTATAGTTAAACTCCCTTTGGAAAATGTTTTTTGATTAAGATTTGAAAG
TGGTATCTAATGGGTGTTATGTCTAGGGATGGAATAACATTTGTTGGCCTTGATAAACTT
TTGTTTTTGAATTCAAATGGTTTAATTTGACTTGTAAAGGAATTAGGAGTTGAGTTAAAT
AACTTAAATTATTAACTTAGTGTAGTCAAATTCCCAAAACTTTATTTAATTTGTATTTGG
TTCCTTTTATCCACCAACTGTTTATGTTTTTGCTCACCTTTTATTTGTTGTATTTTTATT
GTTTTTAATCGTCTTATACCAAACCCCAATTATCCTTTGCCTAATTGTGTAATTTACACC
AATAGTCAAATACACACAAAATCATATGAATACGATACTCAAACTTTTTATTTTATACTA
TTTACAAGATACTTTGATAGCTTACCAAAGATTCTAACATTTATCCATTTGTGGAAGATG
ATTGATGTCAATTAGTCAAACAAATCTTTAGCGGCAGAGATGGTTCTCATTGATTCGGCA
GAAAAGTTTCTTTCTTTGCCTTTTCCCAATTTTTGCATATATTTGCGTTTCTTATTGTGT
TGGATATTAATTTGACGTGTAGGCCTCTAAAATTCAAGCCACTATCAGAAAATCGATGCT
CAGAAAACTTTCGAGTTCAATTAAGGAAGGTGAAGTTTGAGGATATTGAGAACAGTAATG
GGTTCTATTTTTTACATGATAATATTATTATTTATTAAAGTTATCATTAATTTGGTTTTA
GTTTTGGTATTTTCCTTTTATTCTGATACCAAAGGAGCTCATCCCAATAATAAATATGCA
TGCATATTCCTATATTAACTTAATAACTTCACCGTCATCTTTGTAAGAAAAAAATAAACT
TTACAATCATAAGCTGATTTCATTCAACCCCTCGTGTACCACAATTTTTCAATATCGAGC
ACTACACCAGTTTGATGATTTCATTTTCAAACCACACTCAGTAAAAGGAAAATCCTTATC
ACGTAAAGGAGAATTTTGTGATATTTATTTGTTTAATATTTTATTTAATTAATAATGATT
ATCATTACTTTTTTAGTTGTCAATGGCTCATCACGCCTTTTATTTTGTTTTTTTTTTCGA
GGGGAAATTACAGTTGAACAAGTCATTAGCAACATCATCAACCTAACTTTTTCCTATTCC
CCCTTAATGGTCATTTGTTGGGCTAATTATAAAAAGAACTATTTACTTCTCATGGTCATG
GATGGGCAATTGCTTCCTGGACCCTAATCCAGAAATGTAATTTACATTCAGGATTAACCA
TTTCATAACGTAAAAAAATTATATTCTGGATTACATTTCGGAAAGGTTAATCCATAATGT
AAAATTACATTCGGATTAGGCTTTCCAAAAGATAAATAAATGTGCAGGAAGTATGTTCGG
AGGTGCAGGAATCAATTGCCTCATAACACCAATTACAAACCCATTTTGGAAACTTCTATT
CTTACCAATAGTTGTTCAAATAAGGCCCAAATGTTGAGAATATTGGGTCTTTCCACTGAA
ACCGCACAAATTTGTGGATCAGAAACGAAACTGCCCAAAATTTAAAATACAAATGGACAT
TTTTAAAATTCCATCTTTCAATGCCCACTCTAGTTTGAGTAATAGATTTCATTTTCGTGA
AAAGTGAAGGATGCTAATGCAGTTCCACGAATGGTTCTCAGAAATTCTTAACGTCAAGTT
TCTTGCATTGTGCGGAGATATTAATGAAAACTCAATGTCTAAAATCTGATGAGATCTTAG
CAAAAATTCTGGAGACGGTCACTTCAAAATTGACAGCAAATAATGTAGTAAGCTACCATA
TTGAAAGTTGCAAGTAATGCACCCAGCATGTGTTTGATGCCGTAGAAAGTGGATAGAATA
GCCTCAAGTTGTTGAGAGATAAGAAAGCCAAGGTCCAAAAATCTGATCGACCAACCATTT
TCATTTAGATGTCATTATACATGCTGACATGGCATATATATATATATATATATATATATA
TATATATATATATATATATATATATATATATATATGCTTTTATTGCTTTATACTATTATA
ACAACTTCAAGTTGGATATACAACAAATAACAACTAACGAATAATTGATTCAAGTTACAT
TAAATTTATTTATTTCACCTAAGATTTTAGGTTAAATCTCGTGAATAGAAAAAATATGGA
TGTAAAAGAAGAATTTCAGTAAAGATAGTCAGATCTCTTAACATAAATTAATCATTGACA
AATATTTCATATTAATAACATGATTATAAAAAAAATATAATGATAATTGAGGTGAAGAGA
ATTTCAAGTCAAGTTGGATGGACAAATTTGACTATTTCAAATTTAATCTAAAACCTAGCT
CAATTTATTAAATGAGACATACAAATTGTTTGAGAAAAAAAACGGACTAACGTACTAATC
TATTTCATTACAAGCCGGCCTACGTGGATCAAGTTAAATCAAAATTAAATTGAGTTCAAA
ATTTTCAAATTCAACCAACACATTTTAGCTTATTAAATTAAACGGATCTACTTATTCCAA
CAACCTTAGTTTAAGTAACCTATAGACTATAGTTAAGAAATTGTCACACTTTTTATAATA
TTTTACATTATTTTTGGTGATATATTTTTCCATTATTATTATTACACAAATATTCAAAAA
AAAATCGCACCCTAAAATTTACATCTATTTTATATCATTTTCTATTTCTCTTTATTTTTT
ATTATATCATCTATCAGATTCACAATCATTCCCTTTCTTTTATTTCTCTCTTAAATTCCA
AAATATATACAACATTTAGAGTGTAAAATAAATGTTTTTCATAGAAATTAGCCCATAATA
GAGTTTGTACATGGCTAGATACTGAACTAATGCATATAATTTCTTACCTTGCCAACATAA
TATGAAAAGCAGAAAGTATTTTTTTAAAAGAAAAAGTAGAAAGTATTTTGTACATTACCA
ATACGTACTTTTAAATATAAGGTATCACAAATATAAATATAAATACTTAGAAAAGATTGA
TTTAACTTTAGAGAAGATTTTTATGATAAAACATAAAATTACATACGTTTAAAATTTAAA
ATTTTAAAATATAAAAAAGCGTTTAACTAATAAAAATAATATTTTAATAGTAATAATTTT
TATTTAATAATATTTTAATAGTAATGCTCAAAATTTTAAAATATCTGCTTATTTTAATAT
TTCTTGAATTAAGAAAGAGAAAATACTAATTTAACATTTATCATCATTCATCAAGTAAGA
AAGATACATGAATACATCAAAAGATAAGTACTTTTACAGATGATATTATTTAAATAGCCT
AATTTTTTTTAAAAAAATGTATCTTATCTAATTTCAATTTAAAAAAAAAATGAGCTATAT
TAATTACTAAAGCATCTTAGGCCCGCCGTAAAATTGGCCAAATTATTCATTTTAGGCCAT
TATTCCAAAAACTTTGAGCAACCACTGACTATTATAAAGAATATATCATATTCCAACTGA
TGTACGAATATTCTTAAAACCTAGCACGTTTGTGAATTTTATTCCACCTCTATCTTTATT
TCTTGTGCTATTTTCTTCTTCTTTTGTTTAACCAAAAAGGGTAGCCAAGGAATAAGATAC
CGATCCGATGCCATTTCAGAATCTTGCTTGTTCTCAACTTTCAACTCTCAGAGTTAAAAA
TAATATTCCACTTTGATATAAGAATATGACTATGACATCTATTGTATAATTTAGATCCGA
GAATATGACTATGACATTACGCCAATTAATGTCTTGAGGAGTAGTATGTTTGCCTTCATC
CCTTAAAATAGTTCCTATATATGCTAATTTACTTCCACAATTCAACATTGAAATTGGAAG
TCTAAGATATAGAAATGGAAATATTTAAGCATGTGAAAACTAAGCCCAATTGGAATTCAT
TTTGAATCAGTCTTAATTAATATTTCTTCCCCACAGTTTTGATTTAGACAACTCATTTTT
TTAAGCTTGAAAGTTGAAATTAAGTAGTACTTATCCTTTGCAGCGATGTAATGTATTATT
ACTCGTTAACACCATATTTCAGTTTCTAATATAGATAACACAAACGTAGGGTTATATGTA
AATTTATGATAAGGACGCAACACAGGCTTAAAAATTCATATATCAGTGGTGGTATATGAT
AATGTGATATGAATAGACTGCCTCTTACTTATCTTATCTCATAAGCTAGAGAAAAAGAAA
AAGAAAAAGAAAAAAGTAAAGGTCATGGAAAAATGTAGACAGTGATGTGGAAACCGGAGA
AAAAACAAAGGAAATGACTTGACACAAGAATGGAACTGACTAAGACGCGAAAATAGTATA
ACTTCCCTCAATTATATATATATATATAAAAATACATATATTTGGCAATTGGGATGAGTT
TGTGTTATGTTATTAAACGTAAATATCAAATAACTTCAATTTTGAGTCGTGTGGTTAACT
AATGGCGTCATTGGTATAGAGCCATCAAATTTAAGTTGCTTACTGTCTTCAAAATTAACT
TGAATGCTTAGATTAGGTTGTATCTAAATGTTCCACACTAAGATAAATCTTAGCTTTTTT
TAGCAGCTAAGGCTAATGGATTTTAACCAAAATGATTCATAAGTGGGGGGTGTGTACTGA
TAATAGAGGGGGAAGTGGCATGTTAGTGGTGGATTCTGTTATTTTCTCCTTATTATCTTA
ATTTGTCGGTGTAGCTACTAAGCTTCTTCCTCTGCTGGTTATCCAATATTTGTATTTCAT
CATATACAAATATATTATATTGTGTAACTTCGTTTATGTATTGCTGATTGGTACTTCTGG
TACCTCTTATATAAATTGCCTTTTTTTCTGATTGAAAAAAACAAACAAACATAAGTGACG
GGAACTGGTGTTTGATAGATGATAACAACACCTAAAATAAATTAAAGTCATGGACAATTT
CGGTGACTATATAGATTCCTCCAACCACTAACTATGTATTTGGAAACCACTTCCATATCA
AGATGTGGAAAACTTCCTTATTAATGCGTGACCAATCACACCCTTAACTAATAACTAATT
CAATTCTAAGATAAAAACTTGCTGTAGCTTGATCTACTTGTATTAATAGTTATAGCAATA
ACGATAATTTTTTATTCTACGAACCAACTGCACAAGTTAAAATAATAAAAATAAAAATAT
TACATTAATACAGAAGACCTTCTTTTTCATGTTTTGACTCTTTGATTTAAGTACTTAATG
GGTGTACAACAATAATAACAGCAATGAACTGAATAAATAAGCAAAGAAAAACTGAGTGGC
TATTGACTTATTAAAGTTTAGGGCACTTCTTTTTTTACAACAGAATATTCGGTGAAGTGG
TCCCCATTCTAAATTATGGTACAAACTTTTGCGCAAATATCTAAAATAATATTTTTTTTA
TAATAGGGTTTAATTTTTGTGTTACACACTTAACCTTTTCCTAATTGAACTTTATTCTAC
TTATGAGATTATGATAATAGTATACGTTAAAAAGCCTACGTTGATAAAGGCCTTTTTCAG
GAGTTTATAAGTTTTTTTTATTAGTTTATAAGTATTTTTAATTAAAATAAGTTTATTTGA
TATATGAAATTACTTTTTTGATAATTTATTTTTTATAAGCTACTTCAAGTATTACTTGAA
GTAATTTATAAAAAATAAGTTAACTTATAAGTTAATGATTTTTTTTTTCAATTTTATTTT
TATTATTTTATTTGAAATTTTATTTTATCATTTTAGTCAATTTAAAATTCTCTTATTTTT
TTATTCATTGTATAAAAAAATCATCTATCTAATTTTATATTCTATATATACACAATAAAC
ACAAAGTCAATTTATCAATTGTCATTATTACATTATTTCTTAAGGTTATAAAATCTTTCA
ATTATATTAACTTTATAATTATTTCTTGAATTATTTTGTTTTTCTTTTCCCTAAAATTAT
GACATAATTTGTTTCTTTATATATATTTTTATTTTATCAATTCAATTCATAAAAACAAAA
ATTCTTCTTTTTATTAACTAAACTTCACATAAAATTAAATGTTCTTTTATATTATTTGAC
ATTTATTAGTTAACTTATACGTTAATCTTATTAAATACTTTTAATTAAATCAGTTAATTT
CTAATCTTTTAGCTCTTAACTTATAAATTATAAATTTCCAATTAACTTATAAGTTATTTA
TAAAAAAATTGTAAAACCACTTATCTTTCAAACGATCGCTTTCCTAGCTTAAAATTTCTC
AGCACTTCACAATTAGCATCAGATCACTAATCAGTGATTTTACATATTATCACATATTGT
GGCATAATAAATAATTCATATTGATAAGACTATTCAGGTTTGTTAGTGGAATCCAATGAT
GAAATATTCCTTAACTGTGCAATTGGGAAGCCTGGGCCAAAACACCACCTAAATGGAAAA
ATGGAGCACATAATAAAAAAGAAATGAAATTTGGATCCTGTGTATTTTATTTTTAATAAA
AGAATTTGGATTCTGTTATCAGTGTTATGTGAATACTTGTAATAATCCAAGTTCAGGGAA
ACTCCTATAAAGAGAAAATCAAAATTGTGTTTATGCGATATTCTCCAAAGATGCCAATGT
TATCATCATCAAACGATCACTATACCCCGACAGTATGTCCTGTTATTGGATTTATTCTCT
TCCTTAGTTGAAAACAATTACAGAAAACAAATCGATCGGGCTTGAAAAAATGGGAGGAAA
AGTAGAATGGCATATTGAAAACGAAGTAGGTGACGTATTTGTGCACTTAATTAACTTATC
GATATCCCCACTCAACGTGATAACTTCTCTCTCCTCTCCACTGAATCAAATTGTCAATAC
ACACGAAATTAGGGTATACAAATAATAATGTGTTTTTGGTACACCACACATAAAAAAAAA
ATTCATTTCAGTTTTGTATATAAGATTATTATTCCTCCTCCAGTATTTAAATTTTAACAT
AATTGTATATTCAATTCTTGAATTCAAAATTTCATTTGTTTTTCAATAAATACTTAAGAT
CACATATTAAATCAAAATAATTCTATATTAGTTAATGCACATGGTCGGTTGTATAGTGTA
TTTGAATTGGTGGTAAAAAAATTTAAATCAATATAAAAGTCATTTCCAAAAAATCACAAT
GAGTTGAACTCACATGTTACCAAACGCACCAAGTAAAACTATTTATATCAAGTTCTTCAT
AATTTTTTATTAAATATTTATTTAAATGATGATATGTGAATATTTATAATAATAAATAAC
TTTTATTAAATATATAATGTGTAAAATAAAAAACGGTCTAACTTCTCATGTGTGTCAAAA
GCAACACCAACGTCGCCCAAACATCCCAAATCTTAATCCATGGAAAATGTTCTTTCATCT
CTTTCAAATCGACCATTAGTTAAAACAACCTAAAATTCGATCAAGGTTAATTGCCCAACC
TCGAACTCATTAGTACTAGTGTAAACTATTTGAGCAATTAGTTTAATGGTTAGAGTCGTA
TTTCATATTGATGTACATAGACAAAACCAAAATCTTAATTAAAACTAAATTAATTATCAA
AGTAAATTTAAGATAAATTTGCAACGGAGAACGATATAAAAAAAGTTATATATAATTTGT
GAAATCAAGGTTAGTCTAACGATAAAGTAGGATCTAAACTGACTTTTGAGAGAATTTTTT
TTTTTTACTTAATTTAAAGAGAGATATCTTCTTTCATTTCTCTGGTGGGAAAGAAAATCT
CCTCAAATGGGAGAAAAGTCCTCCTATGAAGGTAGAAGCTCTCCATTGAGGGAGAAGCCC
CCTTAAAGGAGGCTTTGTTGTGCTCTAATACTAGGGTTGTTAAGGTGTCATTGTTGTTAG
GGTTGTTGAGGAAGTACACTTATAATTTTTTTAATTATGTTATTGGGTCAAAGTATTTAT
CAGTTTTGTCGAGATTAATGTGCATTCAAGAATTCCATTGATC
SEQ NO. 34 >gene_1|GeneMark.hmm|179_aa
MESVKFINMNAKHSSIFLLAMIFVLILASANAKIHEHEFVVEATPVKRLCKTHNSITVNG
QYPGPTLEINNGDTLVVKVTNKARYNVTIHWHGVRQMRTGWADGPEFVTQCPIRPGGSYT
YRFTVQGQEGTLWWHAHSSWLRATVYGALIIRPREGEPYPFPKPKHETPILLGNNLKKN
SEQ NO. 35 >gene_2|GeneMark.hmm|849_aa
MLLVEKTNLTSQCFNRISDKKKERWKTHNNNPCRVLFLLCMWSLVVLPSCVRPALCEDES
WDGVVVTASNLLALQAFKQELVDPEGFLRSWNDSGYGACSGGWVGIKCAQGQVIVIQLPW
KGLKGRITDKIGQLQGLRKLSLHDNQIGGSIPSTLGLLPNLRGVQLFNNRLTGSIPSSLG
FCPLLQSLDLSNNLLTGAIPYSLANSTKLYWLNLSFNSFSGTLPTSLTHSFSLTFLSLQN
NNLSGNLPNSWGGSPKSGFFRLQNLILDHNFFTGNVPASLGSLRELSEISLSHNKFSGAI
PNEIGTLSRLKTLDISNNAFNGSLPVTLSNLSSLTLLNAENNLLENQIPESLGTLRNLSV
LILSRNQFSGHIPSSIANISMLRQLDLSLNNLSGEIPVSFESQRSLDFFNVSYNSLSGSV
PPLLAKKFNSSSFVGNIQLCGYSPSTPCLSQAPSQGVIAPTPEVLSEQHHRRNLSTKDII
LIVAGVLLVVLIILCCILLFCLIRKRSTSKAENGQATGRAATGRTEKGVPPVSAGDVEAG
GEAGGKLVHFDGPLAFTADDLLCATAEIMGKSTYGTVYKAILEDGSQVAVKRLREKITKG
HREFESEVSVLGKVRHPNVLALRAYYLGPKGEKLLVFDYMPKGGLASFLHGGGTETFIDW
PTRMKIAQDMTRGLFCLHSLENIIHGNLTSSNVLLDENTNAKIADFGLSRLMSTAANSNV
IATAGALGYRAPELSKLKKANTKTDIYSLGVILLELLTRKSPGVSMNGLDLPQWVASIVK
EEWTNEVFDADMMRDASTVGDELLNTLKLALHCVDPSPSVRPEVHQVLQQLEEIRPERSV
TASPGDDTI
SEQ NO. 36 >gene_3|GeneMark.hmm|512_aa
MAYNFPDVFCWIQSLPPISEWETSSMSLNICSSSSSSCQPRLNLTVSKNNSNNHSSSNLY
FVIIADCNIPIHLWTSKPFKPSSTTITNKTHNNNKLIDDEETISNLFVNFIQAILLYGSN
KNSTPFLRFPNLDSITSNNFSDVFNLSFFTLLFLVCIYEAPAADFRSGCISNLKDHLTGF
QSRQASHKIMKLLGSNLEEHWMRSLNLAVTNWVGELEAHNNPFRTPCPLFSYAFSTIGLW
KVQLYCPLLVMDVENSKSNPASERLQFSLRYHHVEGVLQFNHKVLIKEEWAEIMVDIDNI
RCDVIKLVNESLMSQRGVGAAEKHFPSRISLQLTPTLQDQVLSLSVGKSSENPRKEIGVD
KSVEASFEPSNPLALKVSAGESSTVSLKPWKFEESVYGYSANLNWFLHDSVDGKEVFSSK
PSKFAMLNPKSWFKNRYSSAYRPFNKEGGVIFAGDEYGEKVWWKVDKGAIGKTMEWEIRG
WIWLTYWPNKRVTFYNETRRLEFREIVHLDVA
SEQ NO. 37 >gene_4|GeneMark.hmm|226_aa
MGEIKEECTSLDKSEQEKQLHSKVSKVALYSSGESKAIASACETASSFGSVGPKVSHLKW
GRWYKLRELEVATNGLCEENVIDEGGYRIVYHGLFPDGTKIAGDLDLICPKVFGAPGGSH
YAHYHLVGRRTIILGSSSKARREILAEMGYEFTVMTADIDEKGIRREKPEDLVMALVEAK
RCSALYIGLSVVIDVECLSFYIGLSTTVVENLAFYIDPAVVTVIEC
SEQ NO. 38 >gene_5|GeneMark.hmm|305_aa
MAKKLARFGQFYHCNQMLTHDSWISDPIGTMSHVRASLEKQAVVPIHNAGWNSKSRLFIQ
HLAYGQKHINSHTKGKNTLISCGKTAEAINASKSDASSDNTPQGSLEKKPLQTATFPNGF
EALVLEVCDETEIAELKVKVGDFEMHIKRNIGATKVPLSNISPTTPPPIPSKPMDESAPG
SLPPSPPKSSPEKNNPFANVSKEKSPRLAALEASGTNTYVLVSSPTVGLFRRGRTVKGKK
QPPICKEGDVIKEGQVIGYLDQFGTGLPIKSDVAGEVLKLLVEDGEPVGYGDPLIAVLPS
FHDIK
SEQ NO. 39 >gene_6|GeneMark.hmm|113_aa
MKIGKYKDLSSQLGPPPSIRVIEFKILMAGMPNKLATYPSGGLWLPVEVLSLFVTINDDD
VVEGKFFLGLTVDAKCSYHIQQDLLTMVLFHKPLHELRWEGTCKDTLMLKLEA
SEQ NO. 40 >gene_7|GeneMark.hmm|674_aa
MKPHTTASSFATSLPHVPCFRGTTAARATPSEPHHDSAGGLEFRRVSTSKRRLINLSVRH
ASRVTAASNPGGSDGDGDTRARSCRRGVLMTPFLVAGASILLSAATATARADEKAAESAP
APAAPEEPPKKKEEEEVITSRIYDATVIGEPLAIGKEKGKIWEKLMNARVVYLGEAEQVP
VRDDRELELEIVKNLHRRCLEKEKRLSLALEVFPANLQEPLNQYMDKKIDGDTLKSYTLH
WPPQRWQEYEPILSYCHENGIRLVACGTPLKILRTVQAEGIRGLTKDERKLYAPPAGSGF
ISGFTSISRRSSVDSTQNLSIPFGPSSYLSAQARVVDEYSMSQIILQNVLDGGVTGMLIV
VTGASHVTYGSRGTGVPARISGKIQKKNQAVILLDPERQFIRREGEVPVADFLWYSAARP
CSRNCFDRAEIARVMNAAGQRRDALPQDLQKGIDLGLVSPEVLQNFFDLEQYPLISELTH
RFQGFRERLLADPKFLHRLAIEEAISITTTLLAQYEKRKENFFQEIDYVITDTVRGSVVD
FFTVWLPAPTLSFLSYADEMKAPDNIGSLMGLLGSIPDNAFQKNPAGINWNLNHRIASVV
FGGLKLASVGFISSIGAVASSNSLYAIRKVFNPAVVTEQRIMRSPILKTAVIYACFLGIS
ANLRYQAVLEVDGG
SEQ NO. 41 >gene_8|GeneMark.hmm|271_aa
MSTSSSSQSLKIGIVGFGNFGQFLAKTMIKQGHTLTATSRSDYSQLCLQMGIHFFRDVSA
FLAADIDVIVLCTSILSLSEVVGSMPLTSLKRPTLFVDVLSVKEHPRELLLRELPEDSDI
LCTHPMFGPQTANNGWADHTFMYDKVRIRDEATCSSFIQIFATEGCKMVQMSCEEHDRAA
AKSQFITHTIGRTLGEMDIQSTPIDTKGFETLVKLKETMMRNSFDLYSGLFVYNRFARQE
LENLEHAFYKVKETLMIQRSNGEQGHKRTES
SEQ NO. 42 >gene_9|GeneMark.hmm|776_aa
MAAASEIGNKSMVSLMTMVTQFMDACLKSLGQSSIVSQIMGGVLFGPSMLGNKKILGLAL
FPMKGAVVLDTVSLFGLMFFFFIWCVKMDIATLMKTEKVAITLGISVFAFTLIIPTGLAF
LMMKYIAMDGSLAKALPFLAMSQTLTVFISIAVLLTDLKVLNTDIGRLTMSAAMFADVAG
FILTVILFAILQDQSGSFVRLACILLSIVGVWLLVIFVMRPTIIWMVKHPGRGSVNEICL
VCIFLLVLLSAFVSELIGQHFIMGPILLGLAVPEGPPIGTALMSKLETICTAFLYPIFLA
VNGLQTDFFKINKQSLWIVCVILIVAFFVKIGAVMLPGYYYNLPLKQCFVIGLFLNGRGI
AELATYNMWKRGKLISEQEFALMVASIIVVNCILVPLIRYIYDPSELYQTGRRCTIQHTR
RDLELRVMVCIHNNENLPMILNLLEASYASRESRIEVTALVLVELQGRARPILFANQEQP
HDEMRSMSCNASHIDNALRQYAQQNEGYVSVQSFTSISTFETMYDDICKISLDTGSNILI
LPFHKRWEIDATVEISHRTIQTMNIEVLERAPCSVGILVDRGILSPSPSLLMARAAFYVA
VFFIGGQDDAETLAYASRMVRHECVYVTVVRFLLFGHENSKDRKRDSDLIDEYRYYNAGN
QRFELMNEVVKNGIEMSTCIRRLIDYFDLVMVGREHPDSVIFQGHDQWSECQELGVIGDM
LASPDFVTKASLLVVQQQRIRGTLVKHNVNANPVPNHRDQLVYDIPNDTVDKYDRV
SEQ NO. 43 >gene_10|GeneMark.hmm|74_aa
MANVVEVKVGLHCEDCIKKILKAIKKIEDIETYNVDTQLNKVMVTGNVTTEQVIRVLQKI
GKNATAWEDAQTNK
SEQ NO. 44 >gene_11|GeneMark.hmm|152_aa
MGKYLESAARLEELSRIVSSAAKPNRSKGTLPRSPNRVATPRIANPIGVKVEPAKKMEPL
ECRTPLAKVVADCSKRWFQDTLKEAKAGDSAMQVLVGQMYYSGYGVPRDPHKVTVYTVTN
FVYRGMFGLVKHQEIEIQFGKCAVNGQVKGAL
SEQ NO. 45 >gene_12|GeneMark.hmm|35_aa
MRVPFTTHNSRKNISHAVKYLSTESGRWGEMAESM

What is claimed is:

1. A transgenic plant comprising an isolated nucleic acid molecule comprising a nucleotide sequence selected from the group consisting of: a) SEQ ID NO:2 or a nucleotide sequence encoding the amino acid sequence of SEQ ID NO: 2; b) a nucleotide sequence which is the reverse complement of (a). c) a nucleotide sequence encoding a receptor like kinase (RLK) related to GmRLK18-1 (SEQ ID NO: 3).

2. The transgenic plant of claim 1, wherein the nucleic acid molecule is expressed in epidermis, vascular tissue, meristem, cambium, cortex, pith, leaf, sheath, root, flower, developing ovule or seed.

3. The transgenic plant according to claim 1, wherein said nucleotide sequence comprises: a) the nucleotide sequence of SEQ ID NO: 4; or b) a nucleotide sequence which is the reverse complement of (a).

4. Transgenic progeny or seed from the transgenic plant of claim 1, wherein the transgenic progeny or seed comprises the nucleotide sequence.

5. An isolated and purified nucleic acid molecule encoding a biologically active FRR/CN/SDS resistance polypeptide.

6. A transgenic plant having incorporated into its genome a nucleic acid molecule of claim 5, the nucleic acid molecule being present in said genome in a copy number effective to confer expression in the plant of an FRR/CN/SDS resistance polypeptide.

7. Plant seeds, parts, or progeny of a plant as claimed in claim 6.

8. An assay kit for detecting the presence, in biological samples, of a nucleic acid molecule encoding an FRR/CN/SDS resistance polypeptide, the kit comprising a first container that contains an antibody to any part of SEQ ID NO:3 including SEQ ID NO:21, and antibody to any proteins encoded by SEQ ID NO: 4, or nucleic acid probe identical or complementary to a segment of at least ten contiguous nucleotide bases of the nucleic acid molecule of the odd-numbered SEQ ID NOs:1-4.

9. The kit of claim 8, further comprising a detectable moiety.

10. A method of positional cloning of a nucleic acid that interacts with SEQ ID NO: 1-4, the method comprising: (a) identifying a first nucleic acid genetically linked to a FRR/CN/SDS resistance locus, wherein the first nucleic acid maps between two markers selected from among any of SEQ ID NOs:1-4; and (b) cloning the first nucleic acid.

11. The method of claim 10, wherein the first nucleic acid comprises the Rfs2 gene and the SDS locus.

12. The method of claim 10, wherein the first nucleic acid comprises the rhg1 gene.

13. A method for producing an antibody that specifically recognizes a FRR/CN/SDS resistance polypeptide, the method comprising: (a) recombinantly or synthetically producing a FRR/CN/SDS resistance polypeptide, or portion thereof; (b) formulating the polypeptide of (a) whereby it is an effective immunogen; (c) administering to an animal the formulation of (b) to generate an immune response in the animal comprising production of antibodies, wherein antibodies are present in the blood serum of the animal; and (d) collecting the blood serum from the animal of (c) comprising antibodies that specifically recognize a FRR/CN/SDS resistance polypeptide.

14. An antibody produced by the method of claim 13.

15. A method for identifying a substance that modulates a FRR/CN/SDS resistance polypeptide function, the method comprising: (a) isolating a FRR/CN/SDS resistance polypeptide encoded by the nucleotide sequence of SEQ ID NO:2; a polypeptide encoded by a nucleic acid molecule that is substantially identical to SEQ ID NO:2; a polypeptide having the amino acid sequence of SEQ ID NO:3; a polypeptide that is a biological equivalent of the polypeptide of SEQ ID NO:3; or a polypeptide which is immunologically cross-reactive with an antibody that shows specific binding with a polypeptide of SEQ ID NO:3; (b) exposing the isolated FRR/CN/SDS resistance polypeptide to one or more candidate substances; (c) assaying binding of a candidate substance to the isolated FRR/CN/SDS resistance polypeptide; and (d) selecting a substance that demonstrates selective binding to the isolated FRR/CN/SDS resistance polypeptide.

16. A method for producing an antibody or peptide that specifically recognizes a ligand of the FRR/CN/SDS resistance polypeptide, the method comprising: (a) recombinantly or synthetically producing a FRR/CN/SDS resistance polypeptide, or portion thereof; (b) formulating the polypeptide of (a) whereby it is an effective immunogen; (c) administering to an animal the formulation of (b) to generate an immune response in the animal comprising production of antibodies, wherein antibodies are present in the blood serum of the animal; and (d) collecting the blood serum from the animal of (c) comprising antibodies that specifically recognize a FRR/CN/SDS resistance polypeptide.

17. An antibody produced by the method of claim 16.

18. A method for identifying a substance that modulates a FRR/CN/SDS resistance polypeptide ligand function, the method comprising: (a) isolating a FRR/CN/SDS resistance polypeptide encoded by the nucleotide sequence of SEQ ID NO:2; a ligand of the polypeptide encoded by a nucleic acid molecule that is substantially identical to SEQ ID NO:2; a ligand of a polypeptide having the amino acid sequence of SEQ ID NO:3; a ligand of a polypeptide that is a biological equivalent of the polypeptide of SEQ ID NO:3; or a ligand of polypeptide which is immunologically cross-reactive with an antibody that shows specific binding with a ligand of polypeptide of SEQ ID NO:3; (b) exposing the ligand of the isolated FRR/CN/SDS resistance polypeptide to one or more candidate substances; (c) assaying binding of a candidate substance to the isolated ligand of the FRR/CN/SDS resistance polypeptide; and (d) selecting a substance that demonstrates selective binding to the isolated ligand of the FRR/CN/SDS resistance polypeptide.

19. A transgenic plant originally formed from nontransgenic plants, or progeny of said transgenic plant, which contains:
1) an expression cassette having a transcription initiation region functional in a plant cell of said transgenic plant;
2) a genetically engineered DNA sequence that encodes a GmRLK18-1 in said plant cells; wherein said transgenic plant evidences detectable increases in said RLK activity when compared to said nontransgenic plants which increases the transgenic plant's biomass relative to that of the nontransgenic plants.

20. A transgenic plant according to claim 18 wherein said plant is a legume.


SEQUENCE LISTING (108 sequences)

SEQ ID NO: 1 - DNA, 2659 bp, Glycine max
cacacacactcacacacactgtttttttgttccactaaatcaaaacctcttatctcttactctcattacattcattcttttgattttcgttatggtagtagcagtggagaaaaccaacctcacttcacaatcacaatgcttcaaccgtgtttctgacaagaagaaagaaagatgcaagacacacatgaacaacgttaacccatgttgttttttgtttctcttatgtgtgtggagccttgttgtgctcccctcatgcgtgaggccagttttgtgtgaagatgaaggttgggatggagtggttgtgacagcatcaaacctcttagcacttgaagctttcaagcaagagttggctgatccagaagggttcttgcggagctggaatgacagtggctatggagcttgttccggaggttgggttggaatcaagtgtgctcagggacaggttattgtgatccagcttccttggaagggtttgaggggtcgaatcaccgacaaaattggccaacttcaaggcctcaggaagcttagtcttcatgataaccaaattggtggttcaatcccttcaactttgggacttcttcccaaccttagaggggttcagttattcaacaataggcttacaggttccatacctctttctttaggtttctgccctttgcttcagtctcttgacctcagcaacaacttgctcacaggagcaatcccttatagtcttgctaattccactaagctttattggcttaacttgagtttcaactccttctctggtcctttaccagctagcctaactcactcattttctctcacttttctttctcttcaaaataacaatctttctggctcccttcctaactcttggggtgggaattccaagaatggcttctttaggcttcaaaatttgatcctagatcataactttttcactggtgacgttcctgcttctttgggtagcttaagagagctcaatgagatttcccttagtcataataagtttagtggagctataccaaatgaaataggaaccctttctaggcttaagacacttgacatttctaataatgccttgaatgggaacttgcctgctaccctctctaatttatcctcacttacactgctgaatgcagagaacaacctccttgacaatcaaatccctcaaagtttaggtagattgcgtaatctttctgttctgattttgagtagaaaccaatttagtggacatattccttcaagcattgcaaacatttcctcgcttaggcagcttgatttgtcactgaataatttcagtggagaaattccagtctcctttgacagtcagcgcagtctaaatctcttcaatgtttcctacaatagcctctcaggttctgtcccccctctgcttgccaagaaatttaactcaagctcatttgtgggaaatattcaactatgtgggtacagcccttcaaccccatgtctttcccaagctccatcacaaggagtcattgccccacctcctgaagtgtcaaaacatcaccatcataggaagctaagcaccaaagacataattctcatagtagcaggagttctcctcgtagtcctgattatactttgttgtgtcctgcttttctgcctgatcagaaagagatcaacatctaaggccgggaacggccaagccaccgagggtagagcggccactatgaggacagaaaaaggagtccctccagttgctggtggtgatgttgaagcaggtggggaggctggagggaaactagtccattttgatggaccaatggcttttacagctgatgatctcttgtgtgcaacagctgagatcatgggaaagagcacctatggaactgtttataaggctattttggaggatggaagtcaagttgcagtaaagagattgagggaaaagatcactaaaggtcatagagaatttgaatcagaagtcagtgttctaggaaaaattagacaccccaatgttttggctctgagggcctattacttgggacccaaaggggaaaagcttctggtttttgattacatgtctaaaggaagtcttgcttctttcctacatggtggtggtggaactgaaacattcattgattggccaacaaggatgaaaatagcacaagacttggcccgtggcttgttctgccttcattcccaggagaacatcatacatgggaacctcacatccagcaatgtgttgcttgatgagaatacaaatgctaaaattgcagattttggtctttctcggttgatgtcaactgctgctaattccaacgtgatagctacagctggagcattgggataccgggcacctgagctctcaaagctcaagaaagcaaacactaaaactgatatctacagtcttggtgttatcttgttagaactcctaacgaggaaatcacctggggtgtctatgaatggactagatttgcctcagtgggttgcctcagttgtcaaagaggagtggacaaatgaggtttttgatgcagacttgatgagagatgcatccacagttggcgacgagttgctaaacacgttgaagctcgctttgcactgtgttgatccttctccatcagcacgaccagaagttcatcaagttctccagcagctggaagagattagaccagagagatcagtcacagccagtcccggggacgatatcgtatag

SEQ ID NO: 2 - DNA, 9772 bp, Glycine max
aagataaaattgctaattattggttaagaaaataattgcaccagatatattatataaaatgtcaaaaacgcattccgtacattataaataatattatatacgtcatatttacatcattttttatccttgtttatctcaaaaaagtgtaaatatagagagagtatatatcatatcatataatatgtaagtttttattagtttaaaaaaatagcttgagagtaatgtgatttgtcatgtgctaataaaatatcattttgaatgctcttttatccacatatattaattgttaatgattgaagtttattattattattataatatccttttaacgatgaaagtttgttttaaaaaaatatagatttaagatgtgtttggaggaatttatttatatcttatctgaacttattttatggcatacgtgtaagtatttaagaaaacttataaaattatagtttatgatttatttataaattgttttcaacttattttaataaaattttcaaaataacttataagaacaaattaaattttttatatgaaaataatttaaccttattttcttttcaattataaaaaacaatttacaaataaaagcttatatatatgatacacacttttaagtgtttaagtaagctatctaaaaaaggccgtacagtgtttctttaatgaactatcgatcgggaatgttatatatggaaatatatatacttgagtgaatataggctcgattactccatagtacagtccaataattattagtaaacgaattatacgtttaatttgtatctatatatcttttgttgataattgatgtaatttcaattttaatttaccaaagagagttagcaccacagcgagcatccgttgcctcattagtcattagtacttatcaccgacatctttttgtttgtaaaaggaccactgattcatttacctacatatataatatacaatatgtatgtatacaaaaatcatagtaaggtttaaatgtaatgcttcatgaataagatattctgtgttacagattaagattcgtgtatgataaaatgtttgttattattagagttaaccggcaatttgttcatattgagtctcattaattaccttcttttcacatgttttgttgacatcgagagtgacgatcctaccgagatagataaggatatatatgataacaaattgagataaaaagctctttgcacagtcaattatgattaagaaaaatatcaaatcagttttacagaccgtagctcattaggcagagataattacatgcacgtaaagaaaaaattattgagtcactaaaattgggatagcgaggaatttgagtaatttgaactaagtcataagtttaaatcgtatcgttaaaaaaaatgtagtttttgttactcttttaaatactagtatttttttttttgaaaggttttaaataccagtattattccactaataacctgcctttatttctttatataaagccttctcttaatgaaaatagaatactaattaaataatcgagagaaaaaagatacaaatggagaacaaattatcatgaaaaagttacacattagaaaatatacatgttttagcattgaaaaatacaatggtcaattataaaccaaagaggcccttagttagttagtctaatgtttaagccaccaaatttttggttgataacgtttaaaagtaatagctagatggtctctttcaaagaaatttctgtccatattattcaggtttcaaattttgtttgtaagacgaggaattttggatcttgatgataagaacaagacagggtgaataagttcatttaattaagatggaaagtgcgagtttaacttgagttacgtgtaaggtttcataatcaagtgtacatatgtatatgtattagggtagattaatgatattagctatcaaatttaataaaatgtatatttaatattatttttttatcaacagtaaattttgttaatttaacagttgaatttaaagttttcataaaataaattaaaccccacattatttcaaaaagtaattaatactttgttactacactcttaattatatgcataatgcattatattttgtaataaaaactttatatttacacacgtatgaccattggtgaacctacactgtggcaagtacaccctcattttctaacattcacaaataaaagtttttctaaacagaaaattataataaaatcttataattttatattttatttcatttatatttatatatttatgataaattcctatatttatatatttaaacccactcattttactttttataatttattcacattgattcaagttctaaatctacacccatcgagtgcataaatcaactggcatatattttaacttaatcaaaggtcttgagtttaagttttgaatataaaattaccttatatatttaaaggaagagtttgttatccatgatgattccataagactctctaacaaaattacttccaataaaatatacatgtggtttataaaaaaaaaattccatcaaaattttacaaaaacaatacaaaaagaataaaaatatttttttaaaaaattaattcatttattttgaatacattacttacttttatatatatatatcaacagggacatagtaattcaagactattaatgttgttcacccgtgacacatgtcaactcaatattacacaatcattatcaaatttaattttagaaaatttaatattttttcccattagcatatagtcatttttattggaaaatacattgatgaaacatattatactaattaaaggataaacattataatttataaaagcattcaactatatccattaattgtaaagaaaattttcaattgagaatcgaagttaataattatcaaaataattcttgcttttatttatgaaaatatattgtgtgattcttaattattttcataaatatataaaaatgaatatcatcatatattttgaagtaacttaaaatatatttaatcctaaggttctacatgcttgaacaaacgtcttcatcacaaatctttgtagaaaaagtaaataagacactaccaaaaaaaaaaaaaatcaccaccactacaaataaaaaaggtacgcaaaaagagagcttacactattaccaccctacacactgtcttttatccacatattccttctcaatcggtaaaagaaccaatagctatgatagacatccccggccggactcgatattttttcaaatgttccctcaaatcactgttagttttgatgttaaaacaatttgtttcttggttttgctagtgaaccgcttgatttcatatagcaaaataagttcctttttttttttttttgtaggctagaaaaataagttgcagtagataaaaataaagacaaagcattctgatcgctataattgtaaccaatgtgcaatattaaaggggtgtctgagagcatacaatatcattttgtagccttttatacccatttcacttaatttgcccatgttctctgtccactcgtttgatgtcttctaagtaataactatcagtttcattgaccttgtggtcataactcataactaccatccttgagctaacacaaagaataaagagatatttaggaagataaaattgtgcgaaagtaagaaacattcaattgtaatatgcttcaacaatagtatggccaacagtagtggcgaatctaagactctgactaagcagccataaattaaagaagcttatttacaactagtgttatcggagaatgaaaaattgaagaataataagttcagctataataaactcgagggaggaaaaacaaagaaattcatgataaatagatataacttattaaatttaaggggtgtatttgcacaccctgaattatagagattcttatatctttgagaaaataattaaattgggaaaaaagagataatgactgattgagatttgcctcagaattgttcgttttaatattggtacgaatctaatggttttatcctgaaagatgctcacaagtattgagggactaataaattgtttataaactactactaaatgagatgagactttaaggtgtactgaagcaatatcatttaaaaaatgactactcgtatttgtgttgagaaaatttattttcaatgaaaagaaaatatatacatataagataaagtaattaacataacgaaaggaaataaaatgcaacattataaaaactacaactatataaatgatatatacaactcctagcacatgcattggattgtgaattaattaaaatgttgtatggatggtaaaaattcaaaactaaaccccacacaatttagtgacacagaatataattagcgttgttctttttacagaaaacgacgagaacaaaggtgtcaaaggaaaggagatggatgcatgtggtatgagctcatccaattccaaacatgttgtggaccaaaagcgaagtaccatgaacatgatgatcacgacgattcttctcagattttgggaccgctatgatatgaattgcgactacactactaactcttacgaaccggggtcatcataaaaccattaccatttaccactcttttgaacgttaatgtagcctaaatcttatatccagagaaccagaccctgtttacatttcctttttaaaacgtttctgataaatttctcttgctagtgtctcagaacccagttagctccttcctcaccacgtgacacttcagtgaaacttggagatgccagcaggtttatttcagccagggtctttgtctctcagggcaattcattaatttaaaaaataacatttttttatacatattcatcagtgcacgaggaggagggatagtatgtatcacactttttaattcactttctattgttttctgttagttgaaattcaaatatccctcactaatttgagactgaaacatttcaccaaaaaaaaaaattgaggatggaactttcttttttagttgatcataaattttttcttctaaaatatataatgtggatacatattttttgagattgaaacctaacaaatgataaataagactcacttatttagtgagacatacatgaatttcagagaatattttcctatataggttattagcatttcttttaatatttttttttattgtcttgtttttaaaaagttggcattctttttaaaattgacttttttgagatattgaactattttaataataataataaaattaagttatatagtgtattaaaaagaataagataaaatgtgttttaaatttctcaagattttagtcaaaattagtttcagtctcctctattaaaaatgtgttttaattctcatatttttaaagatatggtgaatttcatttttaatcttgaacagttctttaattttgacttaattaaattcaacatatttcagaaacacgggaaccaaaaccaccatttttagaatccaagactaaagatcttaatgacgtaaaacacaatttacccgtgagaatattaaagctagtagtattgcttttcagtgtgtttcctacggcacattgttgtgtgtggaagtggaagctagaaaacaaaggcagcagaagaagtatggtcctacaaagtgtgtagtagtgaagaagaaatagccgttggtggtggagaggcgcgggtttgcaataaaagaacagcgcgccatgatcctataataaaccctgtcaacaaaaacaagtatgcttcatgaatagttactatttacaaggaaaactagccgttactcactttttcttcttttttttttttttgtaacaaattctgaaccctgcatgttcattctctctctctcacgctcgcaacccgcgcgcgcacctacacttcttttatgtcatcacgtgctccttctcactctccctctctctcactacaaaaaccattcttcaacttgcaacacacgcacacacacactcacacacactgtttttttgttccactaaatcaaaacctcttatctcttactctcattacattcattcttttgattttcgttatggtagtagcagtggagaaaaccaacctcacttcacaatcacaatgcttcaaccgtgtttctgacaagaagaaagaaagatgcaagacacacatgaacaacgttaacccatgttgttttttgtttctcttatgtgtgtggagccttgttgtgctcccctcatgcgtgaggccagttttgtgtgaagatgaaggttgggatggagtggttgtgacagcatcaaacctcttagcacttgaagctttcaagcaagagttggctgatccagaagggttcttgcggagctggaatgacagtggctatggagcttgttccggaggttgggttggaatcaagtgtgctcagggacaggttattgtgatccagcttccttggaagggtttgaggggtcgaatcaccgacaaaattggccaacttcaaggcctcaggaagcttagtcttcatgataaccaaattggtggttcaatcccttcaactttgggacttcttcccaaccttagaggggttcagttattcaacaataggcttacaggttccatacctctttctttaggtttctgccctttgcttcagtctcttgacctcagcaacaacttgctcacaggagcaatcccttatagtcttgctaattccactaagctttattggcttaacttgagtttcaactccttctctggtcctttaccagctagcctaactcactcattttctctcacttttctttctcttcaaaataacaatctttctggctcccttcctaactcttggggtgggaattccaagaatggcttctttaggcttcaaaatttgatcctagatcataactttttcactggtgacgttcctgcttctttgggtagcttaagagagctcaatgagatttcccttagtcataataagtttagtggagctataccaaatgaaataggaaccctttctaggcttaagacacttgacatttctaataatgccttgaatgggaacttgcctgctaccctctctaatttatcctcacttacactgctgaatgcagagaacaacctccttgacaatcaaatccctcaaagtttaggtagattgcgtaatctttctgttctgattttgagtagaaaccaatttagtggacatattccttcaagcattgcaaacatttcctcgcttaggcagcttgatttgtcactgaataatttcagtggagaaattccagtctcctttgacagtcagcgcagtctaaatctcttcaatgtttcctacaatagcctctcaggttctgtcccccctctgcttgccaagaaatttaactcaagctcatttgtgggaaatattcaactatgtgggtacagcccttcaaccccatgtctttcccaagctccatcacaaggagtcattgccccacctcctgaagtgtcaaaacatcaccatcataggaagctaagcaccaaagacataattctcatagtagcaggagttctcctcgtagtcctgattatactttgttgtgtcctgcttttctgcctgatcagaaagagatcaacatctaaggccgggaacggccaagccaccgagggtagagcggccactatgaggacagaaaaaggagtccctccagttgctggtggtgatgttgaagcaggtggggaggctggagggaaactagtccattttgatggaccaatggcttttacagctgatgatctcttgtgtgcaacagctgagatcatgggaaagagcacctatggaactgtttataaggctattttggaggatggaagtcaagttgcagtaaagagattgagggaaaagatcactaaaggtcatagagaatttgaatcagaagtcagtgttctaggaaaaattagacaccccaatgttttggctctgagggcctattacttgggacccaaaggggaaaagcttctggtttttgattacatgtctaaaggaagtcttgcttctttcctacatggtaagtttcgtgtgctgttctttcattaagtgttgtgtgtgctgttctttaattataatttggagttttaccttagtaatctgtataattctaatcggagaacagtacaaacaaaaacacctaaggaacaacaccttagctttaatataccatatcaataagtgaattattttcttgttcatcttgatgcaggtggtggaactgaaacattcattgattggccaacaaggatgaaaatagcacaagacttggcccgtggcttgttctgccttcattcccaggagaacatcatacatgggaacctcacatccagcaatgtgttgcttgatgagaatacaaatgctaaaattgcagattttggtctttctcggttgatgtcaactgctgctaattccaacgtgatagctacagctggagcattgggataccgggcacctgagctctcaaagctcaagaaagcaaacactaaaactgatatctacagtcttggtgttatcttgttagaactcctaacgaggaaatcacctggggtgtctatgaatggactagatttgcctcagtgggttgcctcagttgtcaaagaggagtggacaaatgaggtttttgatgcagacttgatgagagatgcatccacagttggcgacgagttgctaaacacgttgaagctcgctttgcactgtgttgatccttctccatcagcacgaccagaagttcatcaagttctccagcagctggaagagattagaccagagagatcagtcacagccagtcccggggacgatatcgtatagcacaaattttgcattgatttttttgtgccaaatgtagtaggcctactatatatatgttctatgattctttcattcttatattatttttgcctgtttgaatgcttgaatttgtacatactcatactacaataaggtgtagttctggttaattttacctctacctcaaagctggggtgtaattctgtttcctccaaggcacataatagttgaaaatagttctcaggagcattcattgtttattctgcaagattctctttcacggctgctatcttctatgcatgccctgcccataaatgcattatgaagaattgtaacggctgtgtttttggacttcttcaaaaagtttatgttattgccaggtgtatatatcaacatgttttaaagattttcaaacaatcaggttttagatgtgggtttgcatgcatgagattggactagtgcgcttgatgtagtataaaatataaattgtccaatcagcaccctctacatgtccaaataatgggccttatgaaacttaattttttaattacaaactacagtaatctttttgaataaagatttacaaattacaacagacatgtgaagtcgtcatctttcattgccaattctttcaagtttactactattattttcctgcaagcattccacattcacatctgataactatgacagcatctcccaagataatgacttccaagttccaacactggctctgtacatttgaactaattttatatcatttatctattgtgattgaaatataaaattgaagtgatgtgaacaatacgaatcacatcttgaattaaaatatctaacaacgggaacaaataagaggcccagaaaaaagggataaataacggataacaagaaagaaagaaaaaaaaacccaacataattccaacttcaaaattcactcaataaaaagtttaacatgtaaatttacttggaaacaaaactcataagcaaagaaagtcaaagtatacataacca

SEQ ID NO: 3 - protein, 854 aa, Glycine max
MVVAVEKTNLTSQSQCFNRVSDKKKERCKTHMNNVNPCCFLFLLCVWSLVVLPSCVRPVLCEDEGWDGVVVTASNLLALEAFKQELADPEGFLRSWNDSGYGACSGGWVGIKCAQGQVIVIQLPWKGLRGRITDKIGQLQGLRKLSLHDNQIGGSIPSTLGLLPNLRGVQLFNNRLTGSIPLSLGFCPLLQSLDLSNNLLTGAIPYSLANSTKLYWLNLSFNSFSGPLPASLTHSFSLTFLSLQNNNLSGSLPNSWGGNSKNGFFRLQNLILDHNFFTGDVPASLGSLRELNEISLSHNKFSGAIPNEIGTLSRLKTLDISNNALNGNLPATLSNLSSLTLLNAENNLLDNQIPQSLGRLRNLSVLILSRNQFSGHIPSSIANISSLRQLDLSLNNFSGEIPVSFDSQRSLNLFNVSYNSLSGSVPPLLAKKFNSSSFVGNIQLCGYSPSTPCLSQAPSQGVIAPPPEVSKHHHHRKLSTKDIILIVAGVLLVVLIILCCVLLFCLIRKRSTSKAGNGQATEGRAATMRTEKGVPPVAGGDVEAGGEAGGKLVHFDGPMAFTADDLLCATAEIMGKSTYGTVYKAILEDGSQVAVKRLREKITKGHREFESEVSVLGKIRHPNVLALRAYYLGPKGEKLLVFDYMSKGSLASFLHGGGTETFIDWPTRMKIAQDLARGLFCLHSQENIIHGNLTSSNVLLDENTNAKIADFGLSRLMSTAANSNVIATAGALGYRAPELSKLKKANTKTDIYSLGVILLELLTRKSPGVSMNGLDLPQWVASVVKEEWTNEVFDADLMRDASTVGDELLNTLKLALHCVDPSPSARPEVHQVLQQLEEIRPERSVTASPGDDIV

SEQ ID NO: 4 - DNA, 82157 bp, Glycine max
tgcacaatagttatgaacgttcaaagtctgtccaacccaactttgcatgatattcaaccttacgggcatgtaatgaggaaatccccttaattttactaaaataaaaatcaaaacaaggataaaaataaaatacagataaaatccttaagctacaagtctaatcagaaagaaaaaaaatatgatccagaatcatcaaatgtttacaattccaattaatctctttgtaacacgtaatgttaattcttttcgtaaattaaaaaaattcaactacatgttgtgtaaatttacaaaaattatatacacataacaatctcaaatcaaaaaataatttctgaatgctcatccacagaaaggtttaaactgcacgtcttcacaaggtggattccttaatacagtcagtgtatatatataaacctaaaagtaatttattagaaggtgctaataagttggtgaagcaagccaatctttccataaagcaatggtcatggaaggtgatacttggaaagaaagtacctgaatttcttggtaaagcaagaaattatgtaaacaaagacattggcgtcaggaaacagcatctccattttaagtgccaatttatggtgcgtattcaaacatcatgtcaaataaaagaaagaaattcttacgactttgtaccatttcatgctgtaattgaggggtacacattttttttccaacaacttgaggggtacacgttgaacagaacctaaaccgttctcgtcgaataataccgattcgacaaataaaaaatgaataaattatattggcaaaacaaaaaatagaataaattatactttattttccaactatttcttacttttttagttttctctctctctctctataagttatatatttatatacaaaaagacgaaattcgtaaggcaatcttattggtattttaattttctctactgattatgtctaaccatttatacacacacacacatatatatattgtattacttgttaaataaaatcagaaaaatgttgtaatcactttcaaaactgtagttaataaaccttaactaaatcaagcaaaaacaatggataagatggaagtttagtgatacaaaaatatatacaggtatagtgagaataaaaaagtataaaaaaaagttgaggaagtgtgaaatctacgtgaagatgaaggatgaaaattggttaagttaggttaggttagattttataaaatatgtatgagtttgatttgttttttaaggttcgagcttagtttatatatttagttgatatagactacatttaaaagactgacttaaaagtctctttaaaatacataataataacgaaaatggattaagttagatttggtttttttttttctttttactgggtagttagattaggttaatctttataaaacatgaatttgatttgttttttttttcaaaaaaaattaaggttcaagcttaatttatttagttgatataaccactttcaaaaatctgacttacaagactctttagaattcataatagtgacacttgattaagttagattagactttataaaacacgagtttgattttttttttaataataattaaggttctagcttatatatattatatagttgatatagactactttcaaaagtctgacttaaaagtctctttagtatacataataatataaccttttaatttagttaaaaaatttgtccctaaataaattaataaatccaaacttatatacaagttaataggcttaagtcttaaaaaaataatatatatatatatatataaagcattaaaacatttcaatgaaaacaatataataataataataataaatatattattgttattaattcatagattttattattactattatagaataatttgtgtgtatatatataaatatatagagagagagagggtcattttatatgagtgagaaaatttaaatattattatgaattttcaaaattaaaatacacatgccatatgattttcttaaaaaattacgtaacttttttttttacaaaagtaatcatatggttttaaaaactaatttaaataacttatatataactatatcagttaaatttggttcataaaataagtatatcagttattttacaaaattataagtattcataaaataaatacaaaatgataagtaccaagtgtatggatcagcttatgcgatgttgttccaatgtaactaataatcttaaattcgagtattgaatcgaatatgcaattcatttaaatactttaagagataatttgtttgctcgtaataattttatttgacttaagtaaagttttctcatataaaaaatacatatagttataaaaaaaacattttctgactaaatatattttcacaggctccacacaaaaaggaaataacaaaattttaagagaatgtatatttacactcatcagtttgttaaagttaaaattaaaatggagtagaaattgagagaaaagagaggaaatatttaaaataaaggttgatttgtataaataaaatgtgaaggaaagaaataaaaaactggtgagtattactcaaaattatgtttacaatgatgaaaggaaaactgataaagaaagtaaacactaaaaaaagagaccaacttacaactaacatttacttcgagcatagttaattaagtgtttgggacataatattgtttctataactaagtcttacgtagattaaggtttacgagacctatcctaaaaacatgaattgaaactagtatcacctgtcttctgggctcaatcctggggattcataaagacatttcttgaacaatcaaaggggttatatgaatggtttaacgatcattaatgtgatattgattgacaaccgatcaatgctagatatataggcttaaaatcctgtatcagtctacagacgactaatatgatgtaaaattccttagttttaagtgcttttgaacatcaagagacttaaagttccgtgttggttgacaaacaaatggtatgatatataatccttcaacgtaaacacgaaaaaaaaaacttataatctcgtgccaatcatcgatacagtacaaataataaattaaaatgcaattttttttcttgttcttattttttcttatttctcttaaactagatatactatcgaatccattctatttcttatctatttccattattctacttctcccttattttcattactttattcctttcttttatgtttctatccactttatttatcacctatttctttctttcttaccgaatactaaacaagccttgtgatccgaaagcccgaaacaatcattttttatgaaacagcttacactctggtggtgtgttgtgtaaagttaaataagcttttaaaaaaatggtaaattataaggtgagggaccaaaatgtgagattgaaaataaccgtttcatatattattcaaataaataaatggctaaaattgaatcattctccgtatataatgaccacccatttattttattaatatatctaacaattatttttaactccatataggcatttttgaccctcatcttaaaactcacctcaagaaatatatagttattttaattaaattagtactcaacttcaaattaattattagacaagtgttgttttttaaccatttatcaaattaggaactttatgtcacgttatcctaaaatcgttacataaatttttaatgtcacgatacaattttttagaagaaaaatttgtctgaaacccatatgacatgggatgcattagtcaaagtaacacttcctaaatcatcaacttagttagtggcatgcaacatggcgttaacctattttttttctagaaaaaaaaaaacatataaatatcaccagctgatgtcgcgccaccttcaacgcccagcccagtgtaggcgcactatgaaatcaatgcagtcagttttgtcatgtcagacttgcaaaaaaatctccaaaattatatcacacttaataatttttataaagtattatgcataatatttttttaaagttattatgcatagtatctttaaagtttcattaccactatttttcattagataaacttatttaatactttgtgtagggtcatatataaccaaaaagtaatcttgaatgttagatttaaatataaataaatttattttatcttattttattttaataatttttttattcatgatgaaagatattgtaaaaataattttattttcccattgaaattaaaaaaaaattaaatgatatgaaccaaccttttaattaatatgaaattagttatttttatttatatataactttagataacatatttttactcaaaagactaaattaggtgggaatgatggtcagattaagtgttgtggaattattgaagtcttatattcaaagtcctaacttataactatattaaatacttaaaagttttttatcatctataaccatcatattcaattctaaaagaattgtttctagtaaaaaaaaatccgtgactcagaaaaaaaacattacttgggttgatttttgtagcatctaatataattaggtgttgactccaatttttattttgacggaagaaagttcagaatggcgagaaagtgttgaagcaatattggttgtcgacttgtcgtgtctgagacgtgtacgtgtaccttccttacctaaaaaaatgacaattaaaaagtgtttattcgggacgcgcttaaccaacttcacaattttttaagtaccactccgggtgctatgttacatgggcgtcagtctatttttttttttctttttaactaagaaaaattagtattttaaccaattatataattgtgagattaaatatttaaaatatataattaaaatttataataaataaatatttttaaatatattaattatatttgtatttataatgaataatttatatagtaatataaaattatttttagttattgatattaaaaatacagatgaaaaaaatatattgaaagagatgaaaggttgagaatttttttaaaaatattgttattgaaaaattattagaaaaattcattgaagaatagtttttaaaaaattctcattcaagataattattcttatatatatatataaatttataaatgatttcatgtaatacgaaaaattgttaatccacatgacaatcttagcgaagtggcaaaactgccttggtaaaaaaatttcataagataatcccttttaaataattaatttgtaattaatattgtgccatcctcgttaccttctgttcatttagctttaagaacaaatttatgtttcgcatcacgtaacgtgtgtgtttggaagcgttgaaaccacattcaatgaaagaaaaaaaaaaacatattctaagacacaagcaacaaaaggaaggtttctttaacgtcagttaggttgagataaatgcgtacgcaaaaacagttaaatgattagcttttagaaaaagttagaggttataagaaaacatagggaaatgtcactaatgtgttatactttcaataatcaagattttagttttctctttcaaaaacatattaacttgttctctaattaccatttttaatttttaactacatttattataaatactttagtgaaaaactcatagcagctattccagttctgttcatgtaaatatcgtagaagataattgcatttttttccttttctttttctaaaacaagaaacgtgtgaggaatcttaagaattaagatgctaatttaaaaagttgctgagttagagcataaaagttcaaataaaaaataaatgaatagacaaactattaaattattaatagccttagccttgaatctgatgcagacgtgtatggcaatggacagagaagcattaaataggcctcgttacattcaagtttcaaccaaattgacagggaaaatcctctgatactgttgttttcctgaaaccatcacaatttgtttctcaatcatgtcaacctcatcctcttcccaaagcctcaaaattggcatagttggattcggcaactttggccagtttctggccaagacaatgataaaacaaggccacactctcacagcaacttctcgatctgattactctgaactttgtctccaaatgggcatccattttttcaggtaagtcaaaccaaaccaaaccatgcataaatacatacacacttgcaccattttgctggaaatcccacgtggatcaatgatatagtcaaaatagtgtatataaatagagaacaatttttcacttacgagctgattttgtgaagttaaagtctaaaggcaaattctaagacatttcatgttccgtatgtcaaacattgcgcagggatgtcagcgcattccttaccgcggacatagatgtcatagtgttgtgcacatcgatattatcgctatccgaggttgtcgggtcaatgccactcacctccctgaagcgaccaacgctctttgttgatgttctttctgtcaaagagcacccaagagagcttctactgcgagagttgccagaggattcggacatactctgcacgcacccaatgtttggtcctcagactgccaagaatggatggacagatcacactttcatgtatgacaaagttcggataagagaccaagctacctgctctaatttcatccaaatttttgctactgaggtaggttaatatcctttgtcaatacccaccaatcacgaaagaagaaagaatcatttttttttttttttttgcattggaccagtttaattatgttaatcaagaagaaagaaacagagagggtggaagctaagtaacttctgacgtttgcatttgataaatcaagatacaagataaatctatgttgtaaaaaatataagtctcagtcccacatctagcagaggtaaggattaccatcaccttacccttataagttatttgttggtttgagttaggcctaaatttaaacttacaaagtatcaaaggtagcttatcttggatctattaataggtcacttatcatattacctacacacaaaacccaataatgtggaccgtgagagggtgtattgagaaatatcaacacgttgattttgagaggttaagctaggccaagcccaaattgaaagaatctaagcatacaatcctacagccaaggttatagaacctcaacgtcattattgaaaacaaatgcatattaaacaattcaatcttcaattctcccctggactgagtttgaactaattcttcataagtttggaccattgttgcttggtttacaaaagaatcacaaaaatcagttgtgaatgccttacatttatgattcatccggggtacatctatttcactgtgttttgttacttttgtattacgcagggttgcaagatggtacagatgtcctgtgaggaacatgacagagcagctgctaagagccaatttatcacacacacaattggcaggtatgcagcttcctacatatctaataaaccattgagaagcactaatataaatgctcactagatgtaacttttggctgcttcttgatcaggacactgggagaaatggatattcaatccacacctattgacactaagggcttcgagacacttgttaaattggtaaagagttgttaacatttcccctactttctctaaaaattttcctttatatgtggttatgtatcatatggaaaagttacttgcagaaggagacgatgatgagaaatagttttgatttgtatagtggattattcgtgtataacagattcgccagacaagaggtaatggaactgccaacgaaatgcttatttacttttaaattcctttttaaagcactgaaccaagtaccccaagatagactcatgaattgtgaaaaatatgcagctggaaaaccttgaacatgccttgcacaaagtcaaagaaacgctgatgatacaaaggacgaatggggagcagggtcataaaagaactgaaagttgatgcatatttattttacaagatattttctctaactctcaaatatcctcctgcagttccaattataaattactcttatttcagtttccttttaccaaaattgaagttcaattaataaaccaaagagactggtatatgttcaatcacatgcgataaaaatgttccacgttcttgttccgagcagattctttgtaatttcataaagttagagaaaagaaaaaaaaaacagacatttagtcgccaatgcctaaaaccatataataactccacagtttggttctctgaatgaattcccttcattttaatccaaatctcaactacctcttcaattctaaacaaataaattagaacactaccaagtgatcctctggggtcttgacatgagccttctagttctagcttttcaatcaatgtatctacagagcatgtattccattttggattatagagaaataatgtaaaacttttaaccaaatgttaccgcaaatctaaagaaagttcattgctccatgataaattgataatactacataagatgtacaactgctgattttatatatcattttaacaagacttgccaagagatatatcccttaaagccaagagcacttatgtttcgatttgagacacctctatttattccacttacatttgaaaaataaaaatattacattcaccaaactgggaaatgggaaatatcaaaacgtatagaagtgagccgtggaaggaattgtaaacaaattatgacgaaaaccagaacctattcctttgtgcctattttaccagcttttcaagaatagtacaaaattaccaagaaaaaaaaaatgatgcaacgtattttcaccgcatttatttttctctctcatcaatgctggcttcttctgtttcgttgcataaaattgcagaaggattcggaggatctgatgctgatggctctgtctttctaacttgtaggccagtaaagcgcgctaggtcaatccattgctgcagcaactcacgaaaacaaaaaaatttaaaactcctagtcaaatttagaaatgtatgtgcagatgaataattcacataaaactaaattataccccattggttttaaaacaattgctactcttgattcttgaggttttagtctgtttcgaattatgtcactttagaacatgaagacgacattgcttttttctttgtcttttattgtttctctaactaaaaatgaaagcaataatggtggaagaggataatatagtcaaacgaacttaggtttccttgaaattaagaatatttaatgactttattaatcatagtagaaaacattaaacaattattgtgaaatggaagggaagtatattgcaatatggcaaaagcaaatgctaaaacagtgtctttacaatattggctaaaaagtcaaaagaaaaaagttattagaaaatgtcactaatacatttccattgtgattttcaatacatccctaatgtaatttctaataaaaaaaaaattctattattaattcctcaggaccttattacaaatgctaatgagtttccttaagactcttgtcaagggattaaaaaaataaaatatattaaatgaaaagtaatgtattacatgttgtacttttcataaaatgctctatatttttggcttaattatacttttgatcttcttactttttcaattttgtaaactttatccctctatttttttcccacaattttgatctctaattattttaattatccattaacttaacatcatccaatattcgataaatgtgctgacatggcagtgtagaagagtgtcatgtcaacatgaacgtgttgacatgctagcaacacgttaacaagaccctttttttattccttaacaagtaccgtaatggcactcgttagcaacacctttgtaatattaagatcacacgttataagacaagcggaagttattttctttccttaacttgtgatttattgttcctagacaaaagcacaagctgacgcaattataactcctcaaaacacatatttccatattaacacttgaatgtgaaattcaccactttaaaaagaaggaaaaattaaattagatttttgagaagaattatagtgttcaaccataaataaatgaaatccacttactgacgagattgtgaacattgagcattgaaagtgaacaaaagcagatagaagataaggaaaaaaaaactgacctgggttccccaatatgaattgactgtccgtgctacaaaagaaagcatatttacaaagaatgtttgagaagcaaactgttcagaaatccgatgctccactatcccagcaattatctgctcaacaaaaatattataatttccgtattaatacaaaataacagttccaggcattaaatggatgtatatttgttgaatatgatataaccaaagatgtagtcacagaacaatcaaaaatcaattttaagaaagaaagagccttaaaaagttattcagcaaagtgcagatgaagaagtagaaaggaggataaaattacaaaatcagatataatagatattattgaaccgagtaattttttttccacgattacttttaatagcatccagggtttactaacataatatttggttggaaaataatggagaggaaaggagggcaaagatttggaaggaaagggagaatggaggggagtaaaacacctcctctatcaattttggctcccttccaaaattgggagaatttggagaggagaaagttttacatgaattggactaaactatccttaatggttttatcctattatgaggatataataaataataaattattttattttctctcttatttccttgtgaaccaaaccaagtgttcctcccctctactccctttctttgaacaaaatagatagtatacgatgtagtccaatctttcctattatgcttgctacccaccatctacttcaaacactgcctaagtggcccgaaaaacatagtgtacaaattagttattcaagacacagctggtcaagtcctgctttcaaaatgtctacatgaatttccaaattgtatttctgtccatcttcaatactatatattggaaaaaaatcaaggcatcacataatcaactattgagtattgaaacataaaccatcgacgtgacaatatagaagccttcatttcctacagaaactaggttcaatcttccatgaagtctattgctacatattagttaaaatgaaaactttatgcaacaaacaagttctctatatacctgataacggagatttgcagatattccaagaaagcatgcatatataaatgcagttttgagtattggggacctcataatccgttgttcagtgacaactgctggattaaggactttacgaattgcatatagagaatttgatgaagccacagctcctatgcttgaaataaatccaacactagcaagttttaaaccaccaaatacaactgatgcaatcctatgattgagattccagtttatccctgctggattcttttgaaatgcattgtccgggatggagccaagaagtcccattagagaaccaatgttgtcgggtgctttcatctcatcagcatatgaaaggaatgacaaagttggtgcaggaagccacactgtaaagaaatcaacaactgatcctctgacagtgtccgtaataacatagtcaatctcttggaaaaaattttctttccgcttttcatactgtgccaataatgtagttgttattgatatagcttcttctatggctaatctgtgcaagaatttgggatctgccaacaatctttccctgaatccctgtataaaaagaaccagatagtgaagggaacaaagtgatgaatgattaaaagcagaatgaaggtttcattttcaaataaaaaattacctggaaacggtgagtgagctctgaaattagaggatactgctctagatcaaagaagttctgcaatacctctggtgatactaaaccaagatcaattcccttttgaagatcctgaaattgaaaggtaaatatttcctatgttcaccaccaatattggcacacactatcctgtctaaggtatgcacataaaagagtaaacagatgcatagaaaaaagaactagaaagtagaaacacactaactacctaatcatcaaatgcaagtatgcaactaatgtatgccaaattatagaccagaggtactttattttaagagaaagaaaacagaaataacgacctcattatcacaattcacccattggaaaagtttttatgtccttaaattatacattgctgtgcaatgaatctttcctcaaaaggaatatgaatttaaaggaaaagaaagatagcacaaagacagcactacaaagttgcaagcattcaattaaaatcccccacaccagtaggttgagctgcatgatttgtgtcaattaataaaatgcaaaacagagatatcaattaaagggataaggacccatttatttaagcttttaaaaaaatattttttttacatattttatgtaaagttattttatttggttacaataattaaaaaatgtactttatattataaaaagtagttataattttgactttttttcagctgctactcaaagtagcttctgaaaataatcatatagatagatagattctgattttttttctaaaaaaaaacttaaacaaacacactaagaaattttagaaatgatttttcatgaaaaaagttgaaacaaatgggctctaaaatgctcctgaaatgccaaagttaattgcatacaaaaaaaataatcaataggtactggcacaagacacctagtaatatgcgaaatctcttatgtttgtatcaccaaaatggacaatgagaggacataacaacaacaacaccaccaaaaccttatcccactaggaatgagaggacataaagggctaaaaattggaaggagggtctatggggcaagaagattaacagtcaaacaaattagtaactgtaattgttggtttacctgtgggagggcatctcgcctccgcccagcagcattcataacccgagcaatctcagcacggtcaaagcaatttctactacagggtctcgcagcagaataccacaaaaaatcagcaacaggaacttctccttctctgcgaatgaattgtctttcagggtcaagtaatataactgcatggtttttcttttgtatttttcctgaaattcttgctggcactccagttcctctagatccatatgtaacatggcttgcaccagtgacaactattaacataccagtgacccctccatcaagcacattttgtaagataatctgggacatagaatactcatcaactactcgagcctgtgcagaaaggtatgagcttggaccaaaaggaatagacagattttgagtactatcaacagaagatctgcgtgagatagaagtaaagccagatatgaagcctgaaccagctggaggtgcatatagtttacgttcatcctttgtaagcccacgaattccttctgcttggacagttcttaagatctgttgatgagaaaagttcaagtcttaattgtcatctttatgtgtcttgacctaacaatacaagaactgagcctaatcctacaaggtggagttggctagatggatcaaatgaaaccttttagctctatcaaaaaccaaaattgcaataaagtttctcaggttgccctctaccctttcaacagattatattcctttctcatatcttgacatgatcaaactacttgaggcaacttttcataatcatatcctcaatttttttatgtttaactttcagtttaatatgggaaggaatctttgaggattatgtgtttatttccaattcagggtctgttctttgtagactagatttttctagtcctaacgcaaccaaaatccttaagggcaaccaaaatccttaagggcaactttttacagcacatagcttttgcacttattggaagtcaatttgacaagcaccaacaagtatatactatactactccctccattccaaaataattgttgtcctaaattgttttacacagaccaagaaaaaacaatagatagatgaaagagagttgtagttttacaaagttaatcttatatcatcattgattcatttatagattttgtttccatcattaatattataaggaatatacgtgaaaaaatgtaattaaatattatattcaaaactaaaataacaattattttggaataattttttttttcttatacgacaattataatgggacagagggagtaacatttttctgttgctcctaattatagccacaccacaaccataattttcagagacaaaataaacatttgaaagatcaacatgagtttggatgaaatttatgcataccttcagtggtgtaccacaagcaacaagatgaattccattttcgcgacagtagctcagaataggttcatactcctgccatctttgaggcggccaatgcaacgtgtaagacttcaaggtgtctccatctatcctgccatgaaagtcaagtttcaggacaagtaatgcagaattatggaaaagcaatctgactaagacaaaagagcttcagagattaacagaaaatagtgagccagaaaaaagattgcgagacagaaattggtcgccaacaaaaagttgtctcttttataatttttaattgaaattttcttaatttagctaacatgacttcctacggccacaattgcgtttgcagacacttaaaaaacttgatgttgcagcaaaaatcacgttttatttattattgatgtcaattatttaacagttttatgttaggtttaataacagtaggttgatgcaagaggctaaacattaatcagaaattgaaaggcagtgttattacttcttatccatatactgattgagcggttcctgaagattagcgggaaaaacttcaagcgccagagacaatagtttttccttctccaaacagcgcctatgcaaattcttcacaatctcaagctccaattccctatcgtctcgaaccggaacttgctctgcttcacctaaatacaccactcgagcattcatcaacttctcccacactttccctttctctttccctatcgccaacggttctcctatcaccgtcgcgtcgtaaatcctcgaagttatcacttcctcctcctccttcttcttcggcggctcctccggcttcggcgccgaagccagaggagattccgctgccttctcctccgccctcgccgtcgccgccgagagcaggattgatgcgccggcgactaagaacggcgccatcagcacgccgcgccggctgctccgagctcgagtatcgccgtcgccgtcggaaccaccgggattcgaggcggcggtgacgcgactcgcgtggcagacggaaaggctgacgcggcggcgtttggcggtcgagacccggcggaattcgaggcctccgggggcgtcatgaggagggtccggtggcgcggcacgtgcggcagtggcgccgcggaagtaaggcacgtgcgggagacgagtgacgaaagaagaagccggagtgtggggcttcattagtttcgttggcttcagtctggccttatcatcaaccgcaacaaggtttaagttttgtcagttcacttttttcaactgccacacaaaacacgaacaaggattcctttattttcagcattacaagattccataattattattattattattattttggagaaacttttttttttcctttcaaatatcccttttcatagaccattttaattgacaaacaattaaacattaaataaaaaaacatttattatcaaacagtagatcaatttaggtgtgttagtctaaagaaaaacaaataaataacaatgacatgttttgagagatagacaaatattccctctaacataacataaaacacacacacacacatatatatatatatatatatatatatatatatatatatatatatatatatatatatatatatatttgtggtagaaagaaatattagttttagttcgacatattataagatattagtttctttatgtgaaattcatgttcttttattatgtgtttaatttaaaagataaaattatataatctaaaaataaaaaagaaatttttgaattaaaataaaatataagaggtgtaggattcatattattttttcctcaaccaaaaagacaccctaaaaaagttaccaatatactttttaacatgttattttaaaagtatttttattaacttaaatttattaaaaattatatttttttttaatttcatcttttattcaatgaattttccctataatttgattgttttcaataaattataatcattattaaaaaatatataataaagaaagtgtatcaagaaaatatatcaagaggttattcttatttttgcctctcataaaatcctagacttaaaatatacactctactttttgtgttcaataataaattcgtatatttcgtggtcgcaaaaaaattaaatgatattgataatttacatagtcacgcaaactatatatagaaggcctcaaatttagagctctacgtgaagcattggtgtccatagagttacatggtgccaattaccatatctttcattattttggaattttcatgtaagataaaatcgatccgagtcagatgtgactcaggtctgaaacacaggtgccgatccaatatccatgtatgccaataatacgaggcgtctactctaatttgattgaaaataagggggcaaaaagtaaaatatatactgccaaattccaattcaattcaactatacgacattgtctaaaagttagaccaaattgaccaactgaagtgatacctctttgttgatataaaaaactcgtgtgtgctatgaatatttttaaaacaaaataatatttatgagataaaaatatcattattgtcaaataatgatgaaatactatcatgtaatttaagaagaaaaaatatataatagaagatgactttattgttaattatgtgaattctattttattatataaaataaattaaggttatgtttaaaaaaattagttgaaagttaaaaaacaaactaattgataaccaaaaacttttaagttaatttattaaattataaatatttgatagaattgttgttgaagtagataaaaaatataatatcacaaaaatagatatatttatatgatatttatataaactttaatgttttatggacaaaagtatattgaggtattataattttattttttaattaattttaaactcttgtaaattatttttcattatatcttttgtttcaattattagattttttcatgttacatattctattattaatcttgtacaatgtcttaatatttttaaccaagtttgaaataaagttgaaaaaacatgcagtaaatattatacttttattatattaattagtataatagttaaaataaattgtataatataaaaatattcagaaaataataaattattttaacattttttactgtcaatttcgtgaagatgttgaaataattacaatggctaagacaaatagtataatataaaaaaattgtaagaggaatgagtgaaaaataaataaataataaaattatgttatatttaaaaggaataataagaatatttaataaatattttaagaattaaaaaataaaatataaaagcaaaaaattagaggctaaaaactagagttttaaaaaagttactttaaataatgtttcacaaaataataaaagttacaaaaaaatacttatttattaaataattaaacaagtttttcaactaataaaaaaataaaactaacttaaataacgtatcaaacatagcctaaagcgaattttaaaaaaaaaattaaggtggaaaagcatcaaaattcaaagttggtatcaaaattaggattaactaaatttaagcaataatatatgtatccttttcctctcggccccaaagttcatgatcaatcttatcaaacctttttaacttatcaattttgctttcatgcaaaatctagtaagagtaacttcaaattaaatccacgtttgatagtgagactcaagtttaaaatcatatctatcttgctatatgtaataatcatgtttgactgctgagtttgatgggtcaagactttcctaataaaataaaatgtgggtacggatgcttagttttgatgggtgcaaatacaattggaaaaggtatgcatcattaacaatgttttacacgtctaatttctccccctctgattctcaagaggcagggacaaacagatttcacatgcccttttctgggatacaaacatggtttctcactttctcatggtttagccttaaaacatgattcatcgcaatctgcccttaccatttgggaatgtgactgaatagtttggtaactcagaatttgctacaatctggtgataagtaatgactattagatcataattttgttgagaatacaatatttctatatattctaatactacagtgtttctatttctggtttcaaatctcaaaaacaagttatacacaaattcttttgggagaaaataaataataataaaaaggcaagctagcaatcaaactccgcaactaaagatacataacgaggtggtcacagaatagcttatccagtacaatttaagaaattggtatacaaagtatgattttcaacaacgaacctcctttacccaatatttagtcacatttatttgtaacctattaaaaacttttgcgaatagctcccctaataaaaaatgccgcatgattaatcatcaacaggaaaaggctagctcacttgatatcatgaaaagaaggcaagacagcaataagacggtctccataaccaacaggctctgtaaataacaaacaaaaaatagtaaggaattcatcacaactatggatgactgattgagtgtagctgccataattgatggcctaaaatatgtttaaacattgataatttggttgagcattgacgttgaacttcaaatattgcaaaaggacggaaatcgcaatgaataaatcactgaaaaagcatagcagaaattaagaccttaagtaaacaatatttttccattcagtcaatagtcatatactgactagaaaacccatgaaaaccgatatactgtaaattacaatgagctaaattaatttatcatgaatatcctgttactttccatcatttagcaaaaaggtatacaagattcaagatccccagtgtttggtatgattacaaaaaagtcacattattttccactttgttttctgttttaagatatttttgtagttataacttataagagaaaacaagaaatgttttctcaaactctattaagcctagatacttatgcgacccaaatacgggggatacgggaaattcttaaaattcaagatacaacgcaactcagatgcattaacacaaatatacacacacacacaaataaatagagagagacatacatacatactttaaataaatgcacagtatttattaagagacattgattatcttacactaatacataactatatcagtggacgatgatcataattcacaaaagcaatacctatgataataacaaaataaaaaaacaaaaaaacagtgacatatgtttccattactcataccagaatatattattttccaacatgctacaaaatcttccccctaacttgtgtcaaaatgtcacaaacatcatcatttgtcctactcaaaagaagaattgactctatatgattgcctagtagtaatatttagtatttactatagctttaaagataaagctgtattgaattttattcctattcaaagtactggagccattctcaaccataccaatctacaaagttttggaaaaaaaaatagaggatactctttggaattggataagtacaagagtatcatatgcgtatcggtgttgcatacaagtacagcatagatactttgtcatttttggagtatcaaggcttcacagctcaaactaaacaaatcaaacccaacatccccacgttttaatatgataacagcatgctagccataaagcaattaggcatccatgatactagagtatcataaatacaggtcagaaaatcagtactaagtgttccaagatggtaaaattcatttttaatgcttatcactgtgactacgagaatttagaattattaggattgccaaatcatttttgccatattttattgctagaggcacactattgctttaactatttcaattttggatgaacagcacggctatcatcactctttctttccccagaagccctgtattactttagtaccatgtaaataaatctatacattttggtaacaggtcatagaaattattatacctccatcctcaacaagtagcttcaacacttctccagccacatcagactgcaagatttcaaaaattaaactagtcagaagtagtaaatatttaggaaggaaccagaattacagaaacagaggcatcaccctgataggaagtccagtgccaaactgatccaaataccctatgacttgcccttctttgattacatcaccctagaattcaattaggaaataaatatattgaaaagaatttgtagtcagttcaatgaaagtgaggtcctcaaacaacttgatgcagcaactgtatgatacaaaatatattaataactacaccagcagaaaaatataggtcaatctatatttgggaaccaaataatatttaatttgtatctgatagactcaagaaattataactaatttggaagaaatggatacctagtattattaaaacaccaaaacacagggcagattatagtagctaaagaggaagaagctaactagtcaaagtgtcacactattcaacactacaaaggaccaatccccttttagagagcctgacctttctcacccaagagctacccaagagaatacacaccctctcctccatatcccctcccatataacacaatcctcaccaactaagcacctacctgacaattccctcctaaccaactctctgctcatcagggttgattctcttctctttccaagactttgggcttttgttttgactaagccaaatttctatctgctggcctggtccaacagtatcttttacagacaagtttacaaaatattcgtatttgttagaatttattgatattcctattatgtccccactgtgtgcaaacatttagaaactaatattacaattaacagtttttgtgaatgcagcaaaactaaatatatatgatatagaaatcaacaaaactgaaaaattatatgcaaagttcaattgaaaagaaaattgattaccctttttgtggtaataaatataatgataaactaggtaggttacagtttggatttgtgatcaattgaagatctagatgctaattggtcataactacaatattttttgcagtgattctgtgcctcacatcagtcacgtgtgtctatataacttgttcttaaagtaaatattaaaataatataaaaaatattagaaatttaaattatatttacattttttaaatgtattagtaagttttgtttatatcaataatcaattttttaagataaaaatttacaaaaaagtaatacagtaatagaaaaataattatcaatattgtataagctgagactatcattgctaattattatcagcttttcttttttatagcatccagttttttcagttaaatacttaaatttatttcaaaagccaacatatgcatgtcagcaaggtcacatcaggagactaggccgacaagcaagcaggctgatacatagactgcaactattagtttcagcactgcaaaatgttagtgaacaacaaacacatgcaccaaatccaatggtggagttcataatgagaatccagatggagggagtatcaagtgaattatgtgatctagttcataacaaattgatgacataaaaacaccagagaagaggatggatcttgacaagcttatcacatgtacctctttacagataggaggttgcttcttccctttcactgttctacctcttcggaataagccaacctaatgagaaagaaagatctgtgatagctaactctacatagctcaagtcagagataattagtgaaagcagaagttaaaacatatattattgatggatgccataccgtgggagatgttactaagacataggtattggtcccagaagcctccaatgctgccaattttggtgatttctctttggaaacatttgcaaatgggttgttcttttcaggagatgattttggtggtgatggtggcaggctattgggtgctgattcatccataggtttacttgggataggtggtggtgtagttggtgaaatgttagacaaaggaacctttgttgctccaatgtttcgcttaatatgcatttcaaaatctccaacctataacaatataagccaaataaggttgtgtacaaggtcatccagctaaaagcatgcttcttttgatgaaccagacagaaattacagatttgaagttatattaaagttattaggcacaatgttgtatgaaaacaaagctgagctgacacaattttttatgatcaaatttgcattacaagaaaaactatatattcagataggacattaggcacaatagcaccatattctcaagtttcaattacaatgtgctaccattttgataaaaacataaataccattacctcatttaataatctataagcctgtgttatcttaagtgaattaaaatcatcctttgacataagttcaatacacagtacatggcaggctatagacatatccagacactaattacgaattgcaaagaaccctaccatataacattttgctttagaacatacacttaaagtttttttcatccttgttattattatatttaaaatactgcacttcaaattttacagaagcagtaaacaagagagaagaagatcacctttactttcagttcagcaatttcagtctcatcacagacctctaataccaaagcctgataataagcaaaccagacataagttgtggtttatcagattcacattgaaacatggctgttaacgaatgcaattatgcagtcttggataaaaaccagctatcataagtaaaaaataccttgtatttatttcttctaataactaacataaggtatttgtattcatgagcatacaattgccaattcccaccaaactatcatttttcactaataagataagattttcaggaaaataaaaggaaagaatagctatagacctcaaatccattaggaaaagtagcagtttgcaaaggctttttctccaatgagccttggggggtgttatctgaagaagctgcaacatcataaataattgtaaggtcatggtttacgaatgttaaagtaaaagttgttatagggaagctattctgatagccaactataacaaagctgtatatatcatataaccttcaaacagacattcacaattaccataataataattagcaacagagtagaatgatttcctactattgtgtatctatgtaaataacataataccttaaaaataaacagataaacaataaaaacaacatcttttgtttgtaaggattttcttctatcttctttctttccaacaagtatataacggttcggcaacattatctttgatcatgtatataaaaatatgaaattgcagcccaaaaattttgcaactggttaatgtatctcaaaatcttaagaataaaaacaacttaaagtttattactaagaagataattacactaagaagtaagaacaaataagagtatcagacttttctcattcaaacaacagccaaatagaacaaaaacataaggatataattcatttcacaatcaatataaacccaccatcagaattggatgtgttgatagcttcagctgtttttgcagatgaaactagtgtgcttttccccttcatgtgggaattaatgtgcttctgaccatatgccaaatgctggatgaaaagcctacgtttagagttccatctggcattatggatgggaagtacagcttgcttctcaaggcaggctcgcacatgggacatagtgcccatgggatctgaaatccaagagtcatgattaagcatctggttgcaatggtaaaatttgcccctttaaaaatataaaaaacattgaagtattacaaaaatattgtattatgatttatgaaaattgaccctttaagaaggcattaagaatctttccaaaaagaagtaagcctatttcaaaaaggcttggaaaaagaacggaacacattgttttatttaacagaaatggatcaagacaagaaaaaaaaaaaaaaccaacataacacaaatatttctgacaagtgtttccaatcaaaaatagtatacagcctatgggtaccaccaaagttaataaataataattttaaaaaaagaaaaacacagtcccttgtgtcctactatatgacccaacagaatgccaattgcatgctcttaggtgattgcagagcatcctttggttatatatatatacatatatatatatatatatatatatatatatatatatatatatatatatatatatatatatatatttgtagaaacatgaaggatacattcaactgccttcatgaaaatgttatgctcgtgattctgataaatacgtgacacttagcattagcataaaataacctgtcaatgctacctcatttttccgttaatttctttggtgttattcttcaactgtttttcaatttgatttttcctcatatatgtcacataaattaaagcaaataaaaccgaaaagcaagagagcaagatcagaagtcgcaaacacacgagcagagatggcagtcggcaaagcacgttcataacaaaaaaaaaatgcaggtagagatgaggagagagagagagttacagtgaaaggaacgaatggcaggcgaggattccatgggaagaaatggaaatggaagaatgggagggaaaaacaatggaggaggagaggaacttatagagaagagagaatagccaagttgagttaagcgaatgaggaaagaggtcgataaattagttgcactgtgtctgctttgagatttccgcccctctaatcaccttctccgtttcaatctaggaacattgcctcgctaacgtgcgccggtgtgtgactagtgcttccctcctccctcttacagtcttacgtgggacccacccctccaggcaggtaggtttcatggacagccatacaatgaatagttcaaaaagtctaatttagtagtttcttgttactatattttttttatgcagtccacactaataaaaaattagatggttggaaaacaaatcttattacaagttttataggtaaacttgaaaaactctatgttataagacctttttctcactttggtagtagtctcttattcaagttagataattcttcttatcttaataataatatttttttttatagtgatagtcatggatgttatttagtgggattttattatcccctctctccacctactctttcattatagtaatgcattcttcaaagagtcaaaatacatttcattacttccaagaataaaccttttaattttggatagatttattttttagtcttttaatttatttattttttagatttaatttggtccttcagtttttcagaattcaatttaattctctaattttttaaatcgatcaaatttggtttttcaatctaaattataagaaactatattttgtgatggtttaaaatcgtcattaagtgttcttaagctaccacaaaaagcacatttccaaaaaaataaattaattttaaaaattataagatcaaattgaatcaattttaaaaattaaaatattaaattgaaaaaaaaaataaaggatcaaattgaacataaataataaatttgaggattaaaaaactaatttaacctttaattttttctcacttatattaatattaaaaaattatattgattttcctaataactccttatctcaattaaaatttccaaaaattaattctagcatcttcaaacactactcaccatgaaagttcatcacaaccatctttctttctcttttctctacatcatgttttcgcttcgcaaactttattgtgttcctagtcttagacgtctgataatcttccacaagtattgaactataacacttattgtacttgcaccgttaatagctaacaccaaatgagacgtgtcacttgacttttatatcactaagaaaatttcaacacattgatccagtattagctccatcttgctttaacacttgtttgactagtcacttaagtgcaacaaccaactttgatatcattgttggaaaaataaaccttattagaagtttcctagacaaacacgagaaactctttccattacaagactttctctattacttgggtatggtggtgacttcctttataatggtggtgaatagctccatttataaatgttatttagtgagttttaattatatcatctctctatccacattttcattacgctagtaggattctccaaaaatcaagttacattccattttacgtcacctcttaattttttgctcagttgctttaatatttggaaatttggattggttttcacaacatatactagatataacttttaatgtaattcaaaataacaattcttgataaattgattttcacaacatattcatatatactcaaaatttaaagataagtactacattttatcaggtgggtcaacacattttacctccccgatcatagagtgatcaggaggaaaaaaagaatgaatgaaaggaggagtaaggtgaaggaaagtaatgaaaagaatgagacaacttttaaaaacttaaaattaaggataataaatttatttattgaaataagggtttattttaatcaaataaccaacttttttgtttttttagtatgtttgtctaaattattatttttaaaaaataactttctgtttattttaagaaacaaaacaaatcttatttgcttttttaaaaaatacttatataaaaaatatttattttaattttttttaagtttaaacaaactcatcctaatatgaaccagaaaaccttagtttttgttaacaaaaatgagttaaaataattttggattcattttaaaactatttctttgtctttttagtcattcaaatgatttattgataaaaaattattcaaatatttttgttttcaaatttaagtgaggagtgataaaaacactttttttattggatagaatttattaaaatttacaaaaatcatgagtgaagttagaatgatacatacatattttgtcatttccaataatttttagtgaaaataaattgtattaaaaagtgtgttgctatttttagcactgagagtcatgaacatggatttgctctaggagtgataatttgtggaatcaagtgagggagaaactcatttttcaatttaactttaaaaaccaaaactaaaaaacttaaaactatacattgtattaattagcatgtgttttatatatatatttgagtatggaaggagtactctattcaatgagatgaatatgtgttaacaaaaagattgattaggcgattaagaaagaagagagattcaattcctcttaccactaaaatctaataaactgataattaacatttgttaataataaaaaaatgaaatggacatgcaattaattaggccaatgataaagaatatatttaaaacaaattgttaaatacagtgtgtttgacaatgatatataatcgtgtccatggatcatatcaacctgaaactaatgaaaggatcacagatcacttctatcttccaattaaggaatcacaggtttaaacaatagtagtagttaatttgcacatcactactctggaggcaaggctaagcaacgtcgagatggactatttctcgaaactccaacctcctagtttcactgtagaatgtcacacattttgttagaccaatatgttagccatatccaaccccttatcttccattccgttgttttccctatggctcctttgttcactttccaccacactttttttccatattcatcacctgcgaatataacccatccttccttgttgaaaggtcagtaagcactggagtatcgatttttgaaccatgactttgggttaagcattgcaaacttcgaaggctttgatgataaaacttctttgccatcaacgcagtcatgaagaaaccaattcaaatttgcactgtatccataaactaacggttgaggactctctccagctgacaccttaagtgccaagggattcgatgcctcaaatgaagcttctacgcttttgtcaacaccaatttcttttcgtgggttctctgaggatttgccaactgataggctcaacacttggtcttggattgtgggtgtaagttgcaatgatattctggaaggaaaatgtttttcagctgcaccagcccctcgttgtttcataaggaactcattcaccaattcgataacatcacacctgtccacatggaatattgtaaattatattggagattaagaatttacgttaaaaattaagagtgaatttgtttaaacttataaaaattaatttaaaaatagatatttttttatataaaggtttttttaagaaataaataagatttttttatatattaaaataaaaaaaattatttgtttaagaaaaaataatttataaaaatgttttaaaaaataaaaaattacttattttattaaaatcaatatttattttaacaaataactttaaataaattgaccctaaattgtacacaattaattaagaaaaacagttactcaacatatatatatatatatatatatatatatatatatatatatatattacaatcttatttccttaggtgagatgaattagatagatcacatatcatcatgactcgattaataatctccttaatacagtgaagtaaatatataaagataatgcgattcataaaaagagtgattttgatcacctttgttataaaaagaaaaagaacgatgctttttttttttttgtaaatataaaatttaagtgacttttagatatttccaatctaaatccaaaatattgagctataagaaattgagtttaagttgaacaaaagtatatcatagaaggaagaatttagagaaaagaaaagaaaaaagtacataccacaccttgtaaacaattcaagctaatgagcccaaaattgtacaaatgatgaatttttttttcctaaacttctaataacaacatgggcataagctgaataatggataatttatttgtcttgttaagatgcttatttattggctctatatgtattatctcttaatgggatattaatgggataatattagtggttaattaattagtggttaccttacgttgtcaatgtccaccatgatttcagcccactcgtctttgattaagactttgtgattgaattgaagaacgccttccacatggtgatatctgagggagaattgaagcctctcgctagctggactgctttttgaattctcaacatccatgaccatgaggtgacaatacagctgcaccttccacaacccaattgttgaaaatgcataagaaaacaaaggacacggtgttctgaaggggttgttatgtgcttccaagttccccaacccagttactgaccgcaaggttcaatgaacgcatccacagctcttccaagttagaccctaagagtttcataatcatatgtgatgcttgtctagactgaaaacctatcaagtgatctttcaagttactaatgcacccagaacgaaaatctgcagcaggagcttcatatatgtaaactaggaacaagagggtgaagaaagaacgtctgaaaggttgaaaacgtctgaaaggttgtcggaagcagtggagtcaaggtttggaaatctgatgaaggtagtgctgtttttgttggagccataaagaaggatggcctgaatgaaattagcaaagacattgtaaatggtttcttcatcatctattaacttattattggtgtttttagtagtgctgctaattggcttgaaaggttttgaggtctaaagatggatgggaatgttgcagtctgcaataatgccaagatagaggtttgaggaatgactattctttttgtgtacggtgagattaagacgtggttggcatgagcttgaagaagaacatatgtttaaggacatggaacttgtttcccattctgaaattggtggaaggttctgaatccagcaaaacacatcaagaaagttgttagccatggatcgaatgaagcaacttaattaatataactctctctctctatctctctaattcggttgcattcaggtgtggcttcacatttatttgtagactcttacataatgctatgttatgtactgcaattagcaaatactctttctagtggagaaataataattaaaaaagtggactgattggtacgaccattagtttaattagctccatggagaaaagcaagataaaattgctaattattggttaagaaaataattgcaccagatatattatataaaatgtcaaaaacgcattccgtacattataaataatattatatacgtcatatttacatcattttttatccttgtttatctcaaaaaagtgtaaatatagagagagtatatatcatatcatataatatgtaagtttttattagtttaaaaaaatagcttgagagtaatgtgatttgtcatgtgctaataaaatatcattttgaatgctcttttatccacatatattaattgttaatgattgaagtttattattattattataatatccttttaacgatgaaagtttgttttaaaaaaatatagatttaagatgtgtttggaggaatttatttatatcttatctgaacttattttatggcatacgtgtaagtatttaagaaaacttataaaattatagtttatgatttatttataaattgttttcaacttattttaataaaattttcaaaataacttataagaacaaattaaattttttatatgaaaataatttaaccttattttcttttcaattataaaaaacaatttacaaataaaagcttatatatatgatacacacttttaagtgtttaagtaagctatctaaaaaaggccgtacagtgtttctttaatgaactatcgatcgggaatgttatatatggaaatatatatacttgagtgaatataggctcgattactccatagtacagtccaataattattagtaaacgaattatacgtttaatttgtatctatatatcttttgttgataattgatgtaatttcaattttaatttaccaaagagagttagcaccacagcgagcatccgttgcctcattagtcattagtacttatcaccgacatctttttgtttgtaaaaggaccactgattcatttacctacatatataatatacaatatgtatgtatacaaaaatcatagtaaggtttaaatgtaatgcttcatgaataagatattctgtgttacagattaagattcgtgtatgataaaatgtttgttattattagagttaaccggcaatttgttcatattgagtctcattaattaccttcttttcacatgttttgttgacatcgagagtgacgatcctaccgagatagataaggatatatatgataacaaattgagataaaaagctctttgcacagtcaattatgattaagaaaaatatcaaatcagttttacagaccgtagctcattaggcagagataattacatgcacgtaaagaaaaaattattgagtcactaaaattgggatagcgaggaatttgagtaatttgaactaagtcataagtttaaatcgtatcgttaaaaaaaatgtagtttttgttactcttttaaatactagtatttttttttttgaaaggttttaaataccagtattattccactaataacctgcctttatttctttatataaagccttctcttaatgaaaatagaatactaattaaataatcgagagaaaaaagatacaaatggagaacaaattatcatgaaaaagttacacattagaaaatatacatgttttagcattgaaaaatacaatggtcaattataaaccaaagaggcccttagttagttagtctaatgtttaagccaccaaatttttggttgataacgtttaaaagtaatagctagatggtctctttcaaagaaatttctgtccatattattcaggtttcaaattttgtttgtaagacgaggaattttggatcttgatgataagaacaagacagggtgaataagttcatttaattaagatggaaagtgcgagtttaacttgagttacgtgtaaggtttcataatcaagtgtacatatgtatatgtattagggtagattaatgatattagctatcaaatttaataaaatgtatatttaatattatttttttatcaacagtaaattttgttaatttaacagttgaatttaaagttttcataaaataaattaaaccccacattatttcaaaaagtaattaatactttgttactacactcttaattatatgcataatgcattatattttgtaataaaaactttatatttacacacgtatgaccattggtgaacctacactgtggcaagtacaccctcattttctaacattcacaaataaaagtttttctaaacagaaaattataataaaatcttataattttatattttatttcatttatatttatatatttatgataaattcctatatttatatatttaaacccactcattttactttttataatttattcacattgattcaagttctaaatctacacccatcgagtgcataaatcaactggcatatattttaacttaatcaaaggtcttgagtttaagttttgaatataaaattaccttatatatttaaaggaagagtttgttatccatgatgattccataagactctctaacaaaattacttccaataaaatatacatgtggtttataaaaaaaaaattccatcaaaattttacaaaaacaatacaaaaagaataaaaatatttttttaaaaaattaattcatttattttgaatacattacttacttttatatatatatatcaacagggacatagtaattcaagactattaatgttgttcacccgtgacacatgtcaactcaatattacacaatcattatcaaatttaattttagaaaatttaatattttttcccattagcatatagtcatttttattggaaaatacattgatgaaacatattatactaattaaaggataaacattataatttataaaagcattcaactatatccattaattgtaaagaaaattttcaattgagaatcgaagttaataattatcaaaataattcttgcttttatttatgaaaatatattgtgtgattcttaattattttcataaatatataaaaatgaatatcatcatatattttgaagtaacttaaaatatatttaatcctaaggttctacatgcttgaacaaacgtcttcatcacaaatctttgtagaaaaagtaaataagacactaccaaaaaaaaaaaaaatcaccaccactacaaataaaaaaggtacgcaaaaagagagcttacactattaccaccctacacactgtcttttatccacatattccttctcaatcggtaaaagaaccaatagctatgatagacatccccggccggactcgatattttttcaaatgttccctcaaatcactgttagttttgatgttaaaacaatttgtttcttggttttgctagtgaaccgcttgatttcatatagcaaaataagttcctttttttttttttttgtaggctagaaaaataagttgcagtagataaaaataaagacaaagcattctgatcgctataattgtaaccaatgtgcaatattaaaggggtgtctgagagcatacaatatcattttgtagccttttatacccatttcacttaatttgcccatgttctctgtccactcgtttgatgtcttctaagtaataactatcagtttcattgaccttgtggtcataactcataactaccatccttgagctaacacaaagaataaagagatatttaggaagataaaattgtgcgaaagtaagaaacattcaattgtaatatgcttcaacaatagtatggccaacagtagtggcgaatctaagactctgactaagcagccataaattaaagaagcttatttacaactagtgttatcggagaatgaaaaattgaagaataataagttcagctataataaactcgagggaggaaaaacaaagaaattcatgataaatagatataacttattaaatttaaggggtgtatttgcacaccctgaattatagagattcttatatctttgagaaaataattaaattgggaaaaaagagataatgactgattgagatttgcctcagaattgttcgttttaatattggtacgaatctaatggttttatcctgaaagatgctcacaagtattgagggactaataaattgtttataaactactactaaatgagatgagactttaaggtgtactgaagcaatatcatttaaaaaatgactactcgtatttgtgttgagaaaatttattttcaatgaaaagaaaatatatacatataagataaagtaattaacataacgaaaggaaataaaatgcaacattataaaaactacaactatataaatgatatatacaactcctagcacatgcattggattgtgaattaattaaaatgttgtatggatggtaaaaattcaaaactaaaccccacacaatttagtgacacagaatataattagcgttgttctttttacagaaaacgacgagaacaaaggtgtcaaaggaaaggagatggatgcatgtggtatgagctcatccaattccaaacatgttgtggaccaaaagcgaagtaccatgaacatgatgatcacgacgattcttctcagattttgggaccgctatgatatgaattgcgactacactactaactcttacgaaccggggtcatcataaaaccattaccatttaccactcttttgaacgttaatgtagcctaaatcttatatccagagaaccagaccctgtttacatttcctttttaaaacgtttctgataaatttctcttgctagtgtctcagaacccagttagctccttcctcaccacgtgacacttcagtgaaacttggagatgccagcaggtttatttcagccagggtctttgtctctcagggcaattcattaatttaaaaaataacatttttttatacatattcatcagtgcacgaggaggagggatagtatgtatcacactttttaattcactttctattgttttctgttagttgaaattcaaatatccctcactaatttgagactgaaacatttcaccaaaaaaaaaaattgaggatggaactttcttttttagttgatcataaattttttcttctaaaatatataatgtggatacatattttttgagattgaaacctaacaaatgataaataagactcacttatttagtgagacatacatgaatttcagagaatattttcctatataggttattagcatttcttttaatatttttttttattgtcttgtttttaaaaagttggcattctttttaaaattgacttttttgagatattgaactattttaataataataataaaattaagttatatagtgtattaaaaagaataagataaaatgtgttttaaatttctcaagattttagtcaaaattagtttcagtctcctctattaaaaatgtgttttaattctcatatttttaaagatatggtgaatttcatttttaatcttgaacagttctttaattttgacttaattaaattcaacatatttcagaaacacgggaaccaaaaccaccatttttagaatccaagactaaagatcttaatgacgtaaaacacaatttacccgtgagaatattaaagctagtagtattgcttttcagtgtgtttcctacggcacattgttgtgtgtggaagtggaagctagaaaacaaaggcagcagaagaagtatggtcctacaaagtgtgtagtagtgaagaagaaatagccgttggtggtggagaggcgcgggtttgcaataaaagaacagcgcgccatgatcctataataaaccctgtcaacaaaaacaagtatgcttcatgaatagttactatttacaaggaaaactagccgttactcactttttcttcttttttttttttttgtaacaaattctgaaccctgcatgttcattctctctctctcacgctcgcaacccgcgcgcgcacctacacttcttttatgtcatcacgtgctccttctcactctccctctctctcactacaaaaaccattcttcaacttgcaacacacgcacacacacactcacacacactgtttttttgttccactaaatcaaaacctcttatctcttactctcattacattcattcttttgattttcgttatggtagtagcagtggagaaaaccaacctcacttcacaatcacaatgcttcaaccgtgtttctgacaagaagaaagaaagatgcaagacacacatgaacaacgttaacccatgttgttttttgtttctcttatgtgtgtggagccttgttgtgctcccctcatgcgtgaggccagttttgtgtgaagatgaaggttgggatggagtggttgtgacagcatcaaacctcttagcacttgaagctttcaagcaagagttggctgatccagaagggttcttgcggagctggaatgacagtggctatggagcttgttccggaggttgggttggaatcaagtgtgctcagggacaggttattgtgatccagcttccttggaagggtttgaggggtcgaatcaccgacaaaattggccaacttcaaggcctcaggaagcttagtcttcatgataaccaaattggtggttcaatcccttcaactttgggacttcttcccaaccttagaggggttcagttattcaacaataggcttacaggttccatacctctttctttaggtttctgccctttgcttcagtctcttgacctcagcaacaacttgctcacaggagcaatcccttatagtcttgctaattccactaagctttattggcttaacttgagtttcaactccttctctggtcctttaccagctagcctaactcactcattttctctcacttttctttctcttcaaaataacaatctttctggctcccttcctaactcttggggtgggaattccaagaatggcttctttaggcttcaaaatttgatcctagatcataactttttcactggtgacgttcctgcttctttgggtagcttaagagagctcaatgagatttcccttagtcataataagtttagtggagctataccaaatgaaataggaaccctttctaggcttaagacacttgacatttctaataatgccttgaatgggaacttgcctgctaccctctctaatttatcctcacttacactgctgaatgcagagaacaacctccttgacaatcaaatccctcaaagtttaggtagattgcgtaatctttctgttctgattttgagtagaaaccaatttagtggacatattccttcaagcattgcaaacatttcctcgcttaggcagcttgatttgtcactgaataatttcagtggagaaattccagtctcctttgacagtcagcgcagtctaaatctcttcaatgtttcctacaatagcctctcaggttctgtcccccctctgcttgccaagaaatttaactcaagctcatttgtgggaaatattcaactatgtgggtacagcccttcaaccccatgtctttcccaagctccatcacaaggagtcattgccccacctcctgaagtgtcaaaacatcaccatcataggaagctaagcaccaaagacataattctcatagtagcaggagttctcctcgtagtcctgattatactttgttgtgtcctgcttttctgcctgatcagaaagagatcaacatctaaggccgggaacggccaagccaccgagggtagagcggccactatgaggacagaaaaaggagtccctccagttgctggtggtgatgttgaagcaggtggggaggctggagggaaactagtccattttgatggaccaatggcttttacagctgatgatctcttgtgtgcaacagctgagatcatgggaaagagcacctatggaactgtttataaggctattttggaggatggaagtcaagttgcagtaaagagattgagggaaaagatcactaaaggtcatagagaatttgaatcagaagtcagtgttctaggaaaaattagacaccccaatgttttggctctgagggcctattacttgggacccaaaggggaaaagcttctggtttttgattacatgtctaaaggaagtcttgcttctttcctacatggtaagtttcgtgtgctgttctttcattaagtgttgtgtgtgctgttctttaattataatttggagttttaccttagtaatctgtataattctaatcggagaacagtacaaacaaaaacacctaaggaacaacaccttagctttaatataccatatcaataagtgaattattttcttgttcatcttgatgcaggtggtggaactgaaacattcattgattggccaacaaggatgaaaatagcacaagacttggcccgtggcttgttctgccttcattcccaggagaacatcatacatgggaacctcacatccagcaatgtgttgcttgatgagaatacaaatgctaaaattgcagattttggtctttctcggttgatgtcaactgctgctaattccaacgtgatagctacagctggagcattgggataccgggcacctgagctctcaaagctcaagaaagcaaacactaaaactgatatctacagtcttggtgttatcttgttagaactcctaacgaggaaatcacctggggtgtctatgaatggactagatttgcctcagtgggttgcctcagttgtcaaagaggagtggacaaatgaggtttttgatgcagacttgatgagagatgcatccacagttggcgacgagttgctaaacacgttgaagctcgctttgcactgtgttgatccttctccatcagcacgaccagaagttcatcaagttctccagcagctggaagagattagaccagagagatcagtcacagccagtcccggggacgatatcgtatagcacaaattttgcattgatttttttgtgccaaatgtagtaggcctactatatatatgttctatgattctttcattcttatattatttttgcctgtttgaatgcttgaatttgtacatactcatactacaataaggtgtagttctggttaattttacctctacctcaaagctggggtgtaattctgtttcctccaaggcacataatagttgaaaatagttctcaggagcattcattgtttattctgcaagattctctttcacggctgctatcttctatgcatgccctgcccataaatgcattatgaagaattgtaacggctgtgtttttggacttcttcaaaaagtttatgttattgccaggtgtatatatcaacatgttttaaagattttcaaacaatcaggttttagatgtgggtttgcatgcatgagattggactagtgcgcttgatgtagtataaaatataaattgtccaatcagcaccctctacatgtccaaataatgggccttatgaaacttaattttttaattacaaactacagtaatctttttgaataaagatttacaaattacaacagacatgtgaagtcgtcatctttcattgccaattctttcaagtttactactattattttcctgcaagcattccacattcacatctgataactatgacagcatctcccaagataatgacttccaagttccaacactggctctgtacatttgaactaattttatatcatttatctattgtgattgaaatataaaattgaagtgatgtgaacaatacgaatcacatcttgaattaaaatatctaacaacgggaacaaataagaggcccagaaaaaagggataaataacggataacaagaaagaaagaaaaaaaaacccaacataattccaacttcaaaattcactcaataaaaagtttaacatgtaaatttacttggaaacaaaactcataagcaaagaaagtcaaagtatacataaccaataataataataaaagaaatcagctttatagcattaatttgggatgctctgcttgtatgcaaatgacacaaccttaccctcaagattgcaaaacacagatgagtaacagatgcaatgtgaatcaataaaaagtattgttgcgttgttgatgacacaaccttactcataaaaaatgcattgttgatggctagcattgttgcaaggtattcatacagtttattctgcaacatagagaaaatacaactcatcaacaccaggaaatggttctgttcaaaatcacggattataaaaagttattatctaaatgttacaagcttaagaagatctatcattgtgaaagtctcttatgcacattaatattacaagcttaagaatgctatacaaatgtttgaggttttgatatttaactttttatgatatgctttgatttaatagttgcaaattgccacatttctcatgtcagttactcgtattctcccataaataaataaggcttcttctgtctcaatttattttacttctaaagcaacaatttctttctttctcattttttttgtaccctctgatcagatcgtagtccgatcctcaagccttagcctctacacactttgtgctcgaggctcgatgattgtgtatgcttctgaccggactgtagtctaatctggtggtggatcccaatctgatctgataacctccacagtattgtgcacattataacactgacgtagggttaaccatgtaccgagatccttgggcacagcatcttgatacgctcaattggcctcgattcctgaatatcggagatgggagagtaggcaggtcgcaataaaaggtcataccattaacgtaggatggtaggttaagtaataatacccttgacttgatgctctcaactaggtcaccttgacatacgacaaagccttttctaaaacaattttatacattgaaatttggaaatatgtatgtattgaaattcacaacaatacttttctaactcatatccaaatttaagatcagcctaaaagcccaaattaggggatgcaaagaattctatcgttaaatcaaacacatcagtcaaaaaagaaaacatacataggcacaatcaactgatcaagtataacagtatttcagatattgttagcgactagaaggtttgtatttaatttgcaaaatttaatatgaatactactggttctatttattttataaactggtattagtaatgaaactttaaaatggggaatattttagcaaaaaataaaggttaaaatatgtctatgattcttaatgaatattccaattttatgtttgctttttagtataaaaaaattccgtttttgttccttgataaaaaaggaaatttgtttttggttttaaacactttttttagtccctattaaattacgaatttcctatttattctttgataattttttgtttattattagtcccttcaaaaattactaataataaatattttttaacaggaaataaatacaaaattctctaatttattaggaattaaaaaaatgccaaggacaaaaaaattattattttattaagaattcaatgcaacaaaaaatttacctaaaagcaaataaaaaatttgagtatttcttaggaattaaaaatattttaataaaaataaaataaagatccaaatgatagtgtgataaccgaagaggaatgtctttcaaccactgcctgaccgccaccactgccaacagcctagtatcaaccgaatccacatataccaacaatcttcagacaaacacttctaagttggtgctgaagagacaatatctcatgggtagatcaaattaagagtgctaccaataacaaaatcgggatcatttgactaacaaacagttatgtgcattggatgttctaccatagtacattgctttatgtgaaattcttttaattattcaatattgacatgttcttatatatatatatatatatatatatatatatatatatatacgagggattgtattatctctgaaaaaagattttatcataaaatcataatgatttctcataatgtatctttacattttaaagttagataaataaaattgattttaaattgttagatataattaaaatacataattaatatgacttttaacaaattgatatataaacacttaaaaaaaagtttcatgacgtacggtgtgtattgttggtacaaaaaaaatttatactatcaactaattaaaattattataaataataaaattaataaaaattactataataatctgtaattagattattgtaaaattgttttataatataaatatacagtcttttttctttaagaaaaattgctagaccaagcaatatggaccatgtgctttctgaaaatatataacacaaaaattccattaagtttttttgcacctataagctacatccgctacgtactgcatgtggagcctcatgagtgtgaggatcttccacaggtcactagtttgacatctgaaagctcctcgtgtaaaacgtgaaaacaaataacaagcttggactggtgtacgatttagtgttactagctatcccatgtaataaatatataaatcttgaatcacaaggaatgatgcaatatatggttcctctaatagtaagttatcccaccaaatctgaatataattaagaagttgtattcgtctgaatgttgtgtctaaaagggttgattgatgaatgatggctacatgtgagagtttgataacaacagctagctagccattagccaagccactaactagacattagttttggttggttgtcagacaaaccgttagacctgagaacgaaagcgtattaaacaaaagatgatatgtagacttttaatataaaaagagatggagaaaccaaattgagatttgataggtgaactataaatcatgacagtgcattagacaagttggtagagtttgttactaactcatcagattcttaagaaaggcaaaaatagaaactacaccacatgtcgctagcgataacgtgcaatttataaataaataatggcttcattttcatggttagttataaattaatgggtcacaattcttaatttattaggaacgtatacttcattttgagagtgtataaagttggaagaagaaaagggatatagaaagaataaaaaaatggatttatctaattcatcgtaaatgaaaatgagattaaatcattcaatcttcattgaataatagaatttaaaaaattgtcttattctgaattgtatcattaatattataactatcatatttaatgtattatctttcttatcatttatgtataaaattaaaaatttataattaaaattatattaaaatacataaatatatgtaatagaattataaaaattaaaattacagctatatataatttcctgtcacttagacttgcagtagacaattgtttgtagttaaaaaatatgaactgtagtccggctacgactattaatttcaagacttatttaacagttaataataattttttgtcatctcgtccccatttgtcttgcgtgttaatatatgttataaatagacattgatatatttttttatgtgtatttgttgatacataacaacaataggattgtaattcagattttaatattttattacatgtttatgtattttactataattttaattaacaattataaagttttaattttatacagagaaattaaaaatgatacattaaatgtaataatcataatattaatggacagaattcataattagacaaatgtttaaattgtattggttattgaagatgagattttttattttcatttttacttggggtcttagacaaacctaaaaaaaagagaaaataacacatgtaataattaagtgaagtaaatataatggaaaaagatgacgaaattaagatgagaagaaaatgctatgagattgaaaagatattaagtgtctctttatataaaactcaattaattacgagttaactggtcatgcatgaaagtgtaaaacatttttatgttatcatttaattataaattatagttaatataatttttaaaataattatcataaaaattaataaatttactgtacaagatgaattttaattaaatagtgatataatttttttttacactaacaagtgtataattctttatctctttaattattatctaacttttgattccacggcatgtcaatattttctctctgaccaaaataactatcaaggttagtaaacgaatataaagacaaatccatcatgttcttttgtgtcaaaatgaggccttcttaaagatcacgctcaatgatatgtagttttctaagtcgctaaaatgcatgttaccctcatgaagctataatagggttcagagatagctttagaagttcaatagagcatgtggacctgggagtgaggtcgtatgtcgctataatgctgtataaacttttggtgagcatgcatgaccattttactactggggcttccatagtgggtttcagtgatagtcttcataagttcattagtctttacaagttcaatagagagaatatatggggacctcggagtgaggaggttgaaagtcactataatgctgtataaactttggtgtgtatatgcaccatctgatggccatccaatgtcccctagggacaacagggtacctaattaattggtaccacaacggggagaaaatcaacacgtttgtggaatatacatacctagaattgaagggctagctcaatcaagctaaacttgaattcaactatagaaattaaattaaattgaaatttggttacacgagtcaggaccattagttataattaaaatgcgttagcacaatttctaacgctataggcatagaagcactaatggtgacacacactagtataaaaatacttttaatatcagttattttagatttttttgtttgtgtaagtcaatcaattttaaaagttacttctaaatcaactttaacaaaaactaatgtagaaatgatctagaaaactttttttttaagttctaactctttttcatcaatgttatacatatatatatatatatatatatatatatatatatatatatatatatatatatcccaaaataaccaatcaaataaactacttagtttacttatatgttaaatcatcgacctattacaagagaggaggtctaactcagatgatttattataatgttaagttattttaaatcttttaatatttttatttgatttctatgaataaaaaaaaaatcaacttacccaactttaaagtctcaagtcattggatattatcttttttaaatatatatatatatatatatatatatatatatatatatatatatataaattgaacatttgttgttatattagctaatattgaacatttatttatatcttaaataacatattatctatcaaaataaaatgttataacaaataataattcatttttatttgtatgaattttcataaatttatttattttcaaaaactttgaactcaatgattgtatgaaataatttatattcttaattaatgaattgatcgtatcctatatatgcatagtatatgaaaaatcaattctcttaaagtaacataaagaggccctttcgtttaagtaattgaattacttagacttcaaaaaaaacaattgaggggaatcaaacaattaacagataattaattctagcaaactatattctcctatatattgtcaaatatttgaaaattaaaactgtaagttatgataatgatattatagttgaacacgtggcagtatggtgacccaatgttcgtcaaaaatcaaaatgatgtgtgcctggtacagtcaagtacgtatctttgagtatgacattcttgtgcatgtaattctttgctgatcttatccatgtgtaaaataaaattagttatgttggatgacaggccacctagtttaatacttgagaaatatttttcataagtatctctaactaaactcttgttagatgcaatcaaatcactttagttacttatagtactgttattttatttgctcacgcatcatggaagtctacggtacttatagtactgttattttattagcctaattatccatgtataagaatcattgaataaatagttagttttaccagatagaaaataaaagagggtaaggaacacccaacctatcatgagagctaaagcttcacaacaagcaacgaacagcttttaaccttaaactaggctaatgccaatattaaagaagaaataattaaaattgtaaggctggtcgtgtataaattaaacaaaaggccctctattcaaaccttcatatatcatacctgtttttaattaacgcggactactttttcatataaaaaaaagatcattagaggattaatttaaagcgttttagtttttaattaccaaagagtataattattattaggcgctttgtcccacaatcaatcacctaaacaagaaaaagaaaaagaaaaaaaaagtcaaattggactaatgcaaaagtggcacaatctttgtcttgaactctttaattagcaacaaattatactcttctgcacaaatcacaagaataccttacatgaaaagaatgttaatttgacggtttacattaaattatatgcagttttctgcaggtaattaattttcaagaatttaaggtggttggtaattttcaatagctagcttgactagcaaaggaaagaataaagttaaaatgcttcttgttttggcctttttggattgttatactttttgctaaacggaaatggttatatgaatggtaaaggagataaattgttacatagtctaaaattgttatagtcttaatcaatcttcaaacaatatataatataattttattaactattttttataaattaaaattttcaattatatgtcacataaaaaggaattatgaccataaatatataatagtcttaaatcatgtaataatttgtcagcaaaataagagattaaataaagttagaaggaacacaatcagtggattaattaaaacccgtcatataaactaaattaaatatcaaatctcaaagtacgcataaaaaatctcaatagatttttgttaataattaactagtattcttaaaatattgattaaaaaattaaaagaaaaaatatatttattatataaatcagaaaaatataaaaaaattataaataatacaattttttttctcctaataacaatatttttatttgatgtccttagaatattgggctaacatttaacatataattatacttgggaaaaatttactagaaatagtaataatagattctctaacactttctcctaacatagtctatgattaatttaaatttattgaaaactatgaagttatgagagaattattatttttgtaatttttaagaaatttcaactagagaaaatatgtttaaaagagcctgctgttaaactttctcaaaatttattttcaacctctaaccgcagacttctgaaataagcattcatgcactttattaactagcgggtgcaacaactcctatgggtttggaaccaagttaagtttccctttggggtctgacctcaactaaattaacctaatctgcctaacctcaaaggactttatctctcccccacctctaatccaccctataaaagcaccctctcccactcttacttgcattgcaaccttaaccttcagcattcacactaaggtgttccttgctcgccaaaagatcatggagcctgccaaaaccattcacaacaatgtcaaatactcccccatcttcttagccatctttgttctgatcttagcttcagcattgtcttcagcaaatgccaaaattcacgagcacgagtttgttgtacattctctcactctctttctcttaatttctctggacttattttattcttgttttttttaactcttttccgttagataataattacctagctgttgattgtaatggaataggttgaagcaactccagtgaagaggctgtgcaaaacccacaacagcatcaccgtgaatggacaatacccgggcccaacgttggaaatcaacaatggagacactttggtcgtcaaagtcactaacaaagctcgttacaatgtgaccattcattggtataatatcaagctagcatcttaacttcattttagattatgaagaccctttgacttaattttacacatctgcttaccacgatttatagacatatagatattagtcttacgaaaaaattttttctcactttataattatactactcccttcactcctttttatattaaaatggaaagagtctctctctaccatgtgaaaaataatatataaataaaaacatatatactctaccatgtgaaaaataatatataaataaaaacatatatacactttttacttcttgacattctaattttttttacctttctttctttcccatgatatttatcatttattctctcctctattctcatttatctatctcccaaggtgtgtacattccattagaatgtgaaaatgaaaaacattcacaagcataatgtaaaaaaaataatattatttctcataaccctatatatatatacacgccacataatacgtacgaacgtaagtgatactatcatgaaagttcttgaatggctttcttttcagggtgaatacatatattagtggatagtggtttttgttggtcattgtttcttattattatgtccttaggcacggtgttaggcaaatgagaacagggtgggcagatggaccagaatttgtgactcagtgcccgattcgtccaggaggaagttacacctaccgttttaccgttcaaggacaagaaggcacactttggtggcacgctcatagctcatggttaagggccaccgtttacggtgctttaatcattcgtcctagggaaggagaaccctaccctttccccaagcctaagcacgagacacccattcttcttggtattttaatttccttcttaatttactcatgcatatgcattatttgtaattatagcattcatggtaacatggaggcaactatctaaaaaagaattgagattatttaaaaactaataagtgattgtgatagttgtgattaattaattaatactattgaagcaaagagacaatatatatagaaattgtggttttctgttgtttaattttgcttttggacaaagattaaacggttaaagtgatgatggtgatgatttaggggaatggtgggacgcaaaccctattgatgttgtgaggcaggccacacgaactgggggagccccaaacgtgtctgatgcatacactatcaatggtcaacctggtgatctttacaagtgctccagcaaaggtttgattaattgcttcttaatttcgattgcattaattgaacatgtcacatgtcttgtttaaataaatttactttgcaaaatatttgacataattaaaacaggatatgcagtcataaaaaagagaaaacgacatatgatatgaaattattaaagatgtcaattatttatgaaacaagtcaacaataggttgccttttggtacagcgtctatttcatggcctttctactttttgttctcttttgaagacaaaagtgtcttccccaccaaataaaaagaaaaaaaaatgcagaagacttttaagtaaatatatagtttataaattgcaagttttagcaagaattttttaaaaatataatttgatatttttttcttaaattagaaagaaaaggctaacactttttcttaaaaataattatattataaaattgtctatttaaaaaaacaaaccattgaaatgacttaaagcgaagagattttatacgcgaaacctgcttttaatgattttatgcaaccaacaaggttgcctgcaagtcaaatggaaaaaaggcatttaaaaacataaagttaatcaaacttttcatttctttaatttagatgatgtatcattttaatttcttacattttcttaaaatattaattttatgcattttcaacataactttttttatatatattcaactaatcagaaaatatgataaatataattttaaaataattattgtaaaaatatttttttactataaatattaatttgtaaaaaaatctttacactataaatgatttactattcttttttttacttcacatggattccttcttaaaactttcattttttttactcaaaatctgtaaataaatccaataaattcttttacccttttggtttcatgcagacaccaccattgtcccaatacatgccggcgagaccaaccttcttcgtgtcatcaatgctgcactcaatcaacctctcttcttcaccgtcgcaaaccacaaactcacagtggttggtgccgacgcctcctacctcaaacccttcaccaccaaagtcctcatactgggccccgggcaaaccaccgacgtcttaatcaccggcgaccagccaccttcccgctactacatggcggcgcgtgcgtaccaatccgcccaaaacgctgccttcgacaacaccactacaaccgccatactcgaatacaaatcaccgaatcaccacaataagcattctcaccatcgtgccaaaggagtaaagaacaaaaccaaacctataatgcctccactccctgcttacaacgacacaaacgcagtcacttccttcagcaaaagcttcagaagccctagaaaagttgaagtacccactgaaattgaccagagcctcttcttcactgtgggtttaggtatcaagaagtgccccaaaaacttcggaccaaagaggtgtcaggtattggactattcacctaattctattatcatgcatcaatttaatttgcatgtacgtatcttatcttaagatttcaataaatgtctcatataggaaaaattacttatttatgtttataatccccacaaattttacattttaatccatactcttaaaaattaagtctaatttaatttcttattctttaaaaatgactgatatgttctgataccaaagaattcaaatattaaatatttttattttttgtctttgtattctattttttcataaattctaatcttgctaataatttcaattcatattaagatcggtaaatagaaaatctagaaaaaaaaacaaaaaaagtatttttttttcattgattttattttcaattgatttgtcactaacaaactgattcctcttaaatctcacaaaagtacatgtcgatataaatatgagattataaattcatgatatctattttcgatttttacatataatgttttttttatcttttttagttcctaataagcatttttaaatgtcttatgttcctactttgcatatcagggacccattaatgggacgaggttcactgcgagcatgaacaacgtgtctttcgttctcccgaacaacgtgtccatcttgcaggctcaccacctcggaatccctggagtgttcaccactgattttccggggaagccgccggtgaagtttgattacaccggcaatgtgagccgttcgctgtggcaacctgttcccgggacaaaggcacacaagttgaagtttgggtcgagggtgcagattgtgttgcaggatactagcattgtcactcctgagaaccaccctatccatcttcatgggtacgatttctacattgttgcagagggtttcgggaacttcgacccaaagaaagatacggcgaaattcaaccttgttgatccacctttgagaaacacagtggctgtgcctgtaaatggatgggcagttattcgatttgtggctgataacccaggtaaataaatagggtcttgttaatgggcgtaacattagttaggaaactaaatataaaaaatatttattgtatatgatataagagaatgtaaaaaaatttataaaaaacaacttttcatattttaataaaaaaaatcctttaatttcttactcatgttctaagaacacaaattaatatttaactagtaaatattcactattgagttttaattagataacactctagaaatattatttatttatatatgaatatatactttctttccagttttcatttgtataatgtcctttttataaaaagaaagaaagaagcaaactcaatcatttgatgggtgtgttaattgttaattgtaggtgcatggcttttgcattgtcacttggacgttcacattggatggggtttggctacggtgttgttggtggagaatggagttgggaagttgcaatccatagagcctcctcctgtggatcttcctctttgttaggatatcatttcaaaatattcgttggcccccaacaatcggagttttgcagtttttcttagtttggaagctggttgatgcttccctgcattaattttgggagggttttttgttttgcttcattgctttgttttaattgtacgttctttttctagagagagataaattgggtttggaacctaggagaggtggtgatgatgcggtttcaacgcaactataccatctgaatagtcattgcccaactagttaaattgatgttattttccccccatagctcaagttacaaacagattgcaagttttgaatatcaatatatcagctggcttttttttgggttgcaactcttggtctcttgaatataaatatgttgacatgtattaagtttcaaagtcccctaaccatttgccatcataaactaatacaaaattctcacattaattcacagtatagtgtatacagagggatgcttataatagcatcaagcattaaaatccctcaacgaatgttgtcaacatatggattatgggataaaaatataactacgcttttctgatagagggtggcttttggttttcatcaggctgaatgtgcaagctgatagaggagtattaagatagagtagactattgtaattgagaagcagaaagctactgtcgctgatcatgtgagtgggtggcaaatgtactagtccagtagtcctcttgagttttagcccaataatctcatcaaatgaatctctgggcctgcttcattaatgctaaaagttgcctcttcggcctatgctttttttttctttgttaataaagtgatgaaaataaaggatatattcataaactttatttatgtttactttgtataaatttcttaacaaatacttattgagaaaaaaaaatcaaacttctctcataacataaaattaacttgtgcatttctaactttttagaagttttcttatttaacttccctaaaaaacattacatttgctacagtaacttggtgacagatttcatgttagctctcgcatgatttcaattgtattacaacatttgtactctcagttatccttaataaaatgatatgattttccttgcctaaaaaaaacttggtggcagattttgttgcatagattaattcatctatgtgcctttaagcatttgctagtcatgatggtctataaatgcagtctttacatgtatatttctagattttaacattgttgggttctgtactatcttttggggtctttatttggattcaccttgtgcttaaacctaaaggctattgatcattttattctttttctttttgaatttttttcattaaagttttttctttttataagttgattttaatttttagaagaaacttaattcattttctctcttctttttcctcctgatgaaaaattattcaaattgacttttaaaaagattgtatcatatatatgtgacattttctttttcattattttttatgttcatgtcacataaaatgcaagtctcataaaatgtggttgtaggacaaaaagtgatactatactccacatggtatatataccaaaataaaagtaatacacggaatcatgtgaagcctactcaagtagatgtgcaaaatcttgtgaattcaaattagttgtcttgtttattcattaccttttcaatttttttaatcaccataattaaggcctttcgaatccctttaagtgataaaagaaacgtgcaattatgcaacaaataaattttcgttatgttactatttagtcaaggaggaaaaaaaagtgataagggaagaaacaagggatatttcctgttataacaaacttaaaatggcgactattttgacgacattgcaaatactcatagtacgatataaattttgaatttaatatacaatgaataggcatattcattttctaccccaaaaaagcatactcatttatgtacatttaattttctctccatagaggaattaatgtacaaccatgcataagggatgagcgaaagggacagattattgcaatccagaagcatccaaggaaagttggataaacaaatcaattaatatatataaaaaaaaaacaaaaatgctcctagtagaagattaaaggaagagttggctatatatggcaaaccttttctaactgttttaccctcttctcatcaccgcattgcatcaccaatacgggaacttttccattacaaaactcattggaagccaacatatcccccaaaattcccaactgatctgcattgtccatgaaatttgacatttcttcttctacaaaattccccatgctatgtcgttttcccaccatcactaggtcatagtccttctccattccccgaatcgctttcaacacttgtatgcaatcttccaccacagcctcatggtaaacaacgttaacactatcactactaatatcattcttggcaataaactcatctattagactctcgtccaacgtgctctctaaattctcatcttcattttctacaagtccattaaacctcgaatcttcattggtaggcaaaacaaaccggaacaaggtgacacgtgtgttggggcgttccaacattcgaatccccaacgccaatgcttctctatcatcttttccaccaatgaagaagataccaacatcaaaagacaacttggagctactcccacttagcaccgagtatctatccactagtatccccaaagtaccctttgcattagcgagaaaatttgtgttgaggttgcgaatggtgctggccaggtggcttcctagggtttgatcattttggtgaaagggtataatgagaagatgcactgagttatcttcggctaggttacaaactgcctcatgcatgcttctgtacggagccacgttaacatacgaaagaaccgttacaggaccacttgagttgttggagtagttctcaaaggcacgtaaaatgtggttggtgttggggtagtttacggacaaagattttcgcttgtttttgttcatgggaagaagaatgggtgtgctttttccaacgagctcaatgagatggaccacgtagacgtacaaggggctttgtgtggtagggttgcatgcttctattagggcaatcatgttgtgcacgtgttcgtctgtgtgcacacatgatacgatgttgaacggtgtattttcggttatgttttggattgttttcacactcccttcttgtatggtttgcgtcttacacaccctacggtgcctgtacaaggatttgatcaagggtatgcaaattgatgtcatgaccaccacagacatcaccgctacactgaatacttctgtatcaataacctgcaaaaaggaacgtcatgacataaattagatttaatatttcactaacaatatgtatagcacccacattaaaaagcatgagaatattgaaattcaaatcgcttcaaagaaattatcatatgacccagaactatcatgtattcttgattccgattgtcaatttgtattacatggcatataatcaattttattattttttaatatatgaaaattttgattatatatatcttgaccgaaatcgtagacatgtagtgatgtcaaatcataaaataattccatcaattaaaaacttacatgcataattaatattttttaattgagagacagaaattgagaaattattagacaatatgacatcatcacatgattatgatcttattcagtttctacatgatatataatcatgtttttttttcttaatttacaaaagaaagaataatatatatgactttattatgtgtcatatagatgttaataagaatcgtgaaaattttagaccacctgatcatttctcgaggaaagagcatgaaaaggagacaagcgatttgatttctacctctcataaaatagaagaaaatatataagagaatatgcattaccctgagtttattcattctaccgtaaaagatgagttcgactatacccttgacattcaatatgagcccaagcactacgccatgtttgggcttaatattataggtgggagaaatgagcgcacatgcaagtaccttcaccaagcaccccacaaacaagatagccagaactactagaaccacctcccaatgctcatgaattaaggtcaagtctgtccttgtgccaatcaacaagaagaagaaaggcataaaaaactcatacacgatcaattcacttctctctatgattgttgtggcgagggggggtccatttggcagaaccaaaccatagagaaaaggtcccatgacgaaatatatgccaaatgtgtcactaatagctgccataaccaaaggccccagaagtaacaagacaacgtatgcttctttaattggcttcccaggtggtgttctctccaaaacaatgttcactagtggtcgtataataagaagtaataaaacagcaaatccagttgcacctatcaaaagcacgattagaaatcgcatgctgaattttgagttgaacaggagttccatcgtagtccattgcaatatttcactgatcatggctgaagatagagcaatttggccaagttctgtggctacaaggttgagttccatcaaggtttcggatacaacagcgaaactgctcaaggtgaagatgttcgggaagtggtagattgacatttggttttgattagcattaccgttgggagaatacaaagaaaacaaggttacagtgaccaagaaagaagccaagaagggaaacacaccgaatcgccaacaacgtttcgctgattttaatgttgtcaccacgtccattttcaaacaagtgaggaacacgcaatatgtagtgcctattttggacaacgtatttagaaacagggattgtcttaccggaaagagggctcccaatatttcttcatggcgccctaagaatgttggccccaacagaatgccagcctgtaaaatttaaacgaaattaagattatttatgggtatgttggatagaaaaaacaagaccaataaatataaaaatgattatatatgtgtatgttttctttttttgtgggaaaaggaacgataaacttctaaaaaaaccttaagaaaacacgcttgaagaagcgtctgccataatgccgttaaacaagaaagaatgatcaaacttggtcagctcgtattgaacactagtaattaaaattactaataaattaacctatatatgagaaatgtagaatatattaggagtgaatgcttacgatgacgcagcagatgaatttgggtgtattgataggcctgaggacataatgaagggctttagagagtaaactgactaaaatgatttggaacaatgtgacaggaaccacaaaatcgaatggattatcacccatccacacgcctaaagagcctacagttctatcattcttgagacaaactatcatttgacccgttctagcgtcattaaagaccgtggtaatggaactttcactcattatatatatgtttgtaccaagcaattagtgttgacaagaaaaacaaaaacctttctctcttgggttgttggtgtttgcaacaaacgcaaacacgagcactacctaagggagaacaaaagtagataggagaaaagcatatatgtgcatttttatatgctctaatcaaattggcagataaagcaattatatttaacaaaatatgagctgcgaatgtggtgttagttattacaacggttcaaaaatcaattatagcaatgataactttagccaaatgtattccgagttggagtttcgagaaattttggctaggaaagaaaattgatatgctatcactgagctaaggccagtgtgtaacgaaaaaccaagaagagtgaatttcaattttgtctctttaaggtataattttaaccaaccgtgtcacaaatatcgcacaacaaaagtttgtgtacaaatggtttaatgaagacaggtgtagtcaacgagaataactggttacaaccaacaatgacatacggaatagtggcagggatgatcggaaaaaaaatacagcaaataggcaatagtaaattttaatttgtgcgccgtttcacgataatattttcttgtagaaaaggattctccttgcaatagaggagtacaaaaagatttgtacaaactactactagcatttaataataaaaaataattttgataacaaaatataaattaaaatatgaaaattcatgcatttaaaattttaagtgttgaaatttccttaaaaaaatagtttgattgattgatataatatttctttattttgatatatatatatatatatatatatatatatatatatatatatatatatatatatatatatatatataacgataattgataataaaattttacttttacttttaaatacatatataaaaattaaggaaatagtatgtgattaattttactacttttctataatatagtttaaaataagaaataaaatcaagacacgtacttgtttgctattattatttaaattagaaaatgaaataaatattttattgaacctaaagtgtctttaatcatttagcatggagatcatttggaaaagttgttataatttaactagatgtccgtttgaaaaagtttatgtgtatcttaaagttatattattgtataaagtgtatgtttcgaagatcttattaaaataatttataatatatatctatgataaagactaacaataactaaaaaaaactttgcatcacattgttaatcttttacattaatttaaaatataattcacatattttattttttatttattatgaattttaattataatacatattcaaaattatttatttattataaattttagttaaataaaataaacatttattttgcaaattctacaagctaataaactagtattactaaatgcatagagtatgagcaaattcgtgtaggtggattttgtatattatatatgcttttcgttaaataattattactcgactttagaaggttgaaaaaggtcataaaaatttagctattatgaatctaaactttctttctatttttcttccgatccttttgttttttcatcacattagttatcgtatatgttattttttcttgatttttttcctatctctttattccttctacctaatacactaaaaatgatatatatacatttttccacaaagactcctgcccttttctatcagctaaaattatttatgtacaaataaaaaaggtacaaacacaacatttatttatgaacagataaacgtttttgtgagacattaactgaacctactctatcaagcttattattactactactacttatcttcactccaccacactgtgtcactaaaaccggaaccatccccatacaaaattctactgaagacaacatatcccccaatattcccaatgcatcagcgttctccatgaaagttgtcatttcttttccattcaaagatccatcattgtggcgccttcccaccatcacaagatcatagtttccttccaaactatgcactgcttccaacacctccaccccatcgtccaccgtaatctcgtaccaacaaacgttaccaatgccatatttcatgctcttgaactcgtcaattaacccctcgtccaacatggtatcttcctcttcctcttcacgctcttctcttgtcaaaataattttacaaccacacggtttcttgttcacgataacaaacctaaacaagctcaccctcgtatctgcacgctccgacattcgaattcccaatgccagagcttccctatcgtgggccccacctatgaagaatacacccacgttgaaatacatgttgttgttgttggacgcgcccagccgagagtgccggtccacgagtatccccaacgtgcatggcgcatgcgcttgaaacctagtattcatcttcctgatggaggcagccacgtgtccgacaaggtcaatgttgccgttttcgtgaaaagggatgataatgaaaggcaccatattgtcttgggcgaggttgaaaatggcgtcgtgcatgctcttgtaaggtgccacgttgatgtagggaagaaccttgactggcccacttgagttgttggagtagttttcgaaggcttgcatgatgtggttggtgttggggtaattcacagacaagaattttctgcgaccgtgtctatgttttatgggaaggagaatgggtgcacttttccccacgagctcgataaggtggactgcgtagacgcatatggggctctcttgcactgggttgcactcttctaataaggcagtgatgccacgcacgtttgcttcattatgtacacaacaaacaatgtgaaactctctgtttcttggagtgctttggatcgttctcagttccccttcgaataagctttctgcgtgtactcgagggcgatgcttgtacaatatgttaaccaagggtgttacaatcgcggttataagtaccacacagaacaccaattgactgaatgtatcctcatccaacatctacaaaacgtaataataacataaattagatttgatgtattgtacatacaacgcattttcaatgtttttttaaatgaattagtgcttgagtttatatttcagtgttattttaatccccaaagttatggaagagtcaaataaattttgatttttttatgatttaattcgagtccttcaaattggcgtcttttttctcgttttggttcccagtacttaagtcacaataataataataataataataataaaagtttaggaagaaaaatgagaaaactaattaatttcgggttttatttaaggtttttttagtttcagaaactaaaatgacatatagatacaaatttaaagattaaattcgtcatttactcattttttattagacaaaatttaggtttaagcatgcataccttttgtttcttgaatctagccaaacttatgagttgagttatacccttgatgttcaacatgaggccaagtagcgtgccatgtttaggtctaatgttgtacgctagagaaaccaacacacaggccagcaacttggccaagtctcccgcgaagaaaacaccctgaagggtcaaaaataagcgccaatcctcaagtgcagacaaatccgtgttgatgccaatgtacacaaagaagaagggtagcaagaactcagtggtaagaacttcactcttctccgctagcgttgtccctaagggggggccacttggtacgaccaaaccaaaaatcaagggtcccatgagaaacgttacgcccatcaagtctcccacaccagccatcactagcaccccaagaagtatcaacacaacataaagttccttcacaggttttccaactggggttctcatagcaatcaatttcatggttggtcgaagaacaaagaagttgaagaagacgagcaagcaccagtttcccaacaatgcgattgattttttcacatcaacattagatgtgaagctatgcataacgatgaaaagccatagtatgatgtcgttgatcatggaggaagagagagcgatctggccaagttctgtggctgtgaggttcagttcaagcattgcatcagacacgacggggaagttgctcaacgacattaagcagcttactgagacacgcgcgattgttaaagatgcagaggaaatttgttgggggtgataatagagacataagagtgctaagataaccacaaatgaagcgaggaagggaattactccaagtcgccacgtgcttttggctgctcttatggtcattagtacgtccattttcaatgcaactaagaatacgaaatatacggcgccggttaaggatgccatcactaaatactccgtctgtctcggtggaaataaaacctgccaatacgtcttgttccgccccaaaaatgtggggcccaaaataatgccaccctgcacattttcactaaacacaactttaacattcaaactaaacctatcattataatttacaaatcatacaagaatgaatttcttctgctagaattaacagtttcaaaccttaaattatactaatagatggttaagatttctacttaaaatcatatatatcatttgctttcgttttcaatgctaaaatgatgtcagtaataagcaacagtaataatcacattcatgataatagtgataaaatgatcaccacagtaatatcaatcacaatagtcaaaataataataataatgaccttgatgtgaaaactgctaaagtgaattttatataaggaaatcattctcatatagaaatgataaaattacttattatgagaataaaaacaataaattcttatttgaatggttagatttaaaaaatacatcacttcttattaagtggtcatgtgtgaacattaaattacctttaatctttatcataattactcttttcattcttaaattaagattttttttctaatttctagatatattaattatttttttcttaaatattcttacttaattattttctcatcaaatattaatgagatgaatagagaaataagaaaagaataatttttgaatgataatataattaattaattaataaatttaatgtgattaattaaattaattatttttcttaagacacataaattagttgaaaggtaattgtaataagggacagacggagtgaaataaattgctactaatttcataaataaaaaaccttcaaactatgagcaatcgtggtcattgaaaacaacaaagaaatgaaatcctaactttttatcatgagataaattttcttggcaaaatgatagacagaccaaaatggtgtcttccatgtctctcctccttccacaaacaaacgttaccccatacctcagacactcctcgacaactattggacttgtggagtccaacattggactctttgaagaataattcggaacagcctcaactaccttcaatttggaactataaaccacgttatcacactgccttgtcggagaagtgcacctatccagcttatggcgctttcggctgttttccagcggcgagactaacacattaatagttatgtatttcaagggcaaaaggtccaaactctagcgtgtggaagggcgtgaggtggattccatgggagaagaagtctcgcatcatgttgaggaataaatggtgtataaggagaaggctaagcgtcatgtttgaggagactaatttcttgaagacaatttcaagaaaatcatctttgaatatttgatttttaagacagttttaagaaaatcatctttaaacactcaatttttaagatgatttttataaaattgtcatcatacatcttctattatttacaaaattattactgcctaacattttaagacaaatttttaaaattatcttaaaaaatacgttgtaaaaattattttttagtagtataatcatatttgtcactgttttcattatcattattatcaatatttttattgttatcactaccatcttcacctatcattaacatcatcattccatcgttgtcaacgtgagaaggtggtagcttgataccgacatgatgacaatgatgacgatttgtgataattgttgcaaaaaaagtgaaaagaaaataaggggttaaagagagagagaaaattgtagcctttcaatatttttttaaatttattaaaagggaaaaaaacttttatagctagcttaccaggacgttgcagatgaatttgggtgttcttagaggcctgagaagaaagtaaagcgttcgagagactaagatgacggtgaagagttggcacaacgttacaggaagcacaaattcaaatggacgatccccaatgaaaattcctaaagagcccacatttctatcgtcttcgacacaaacttgccactgcccatgggaatcccaataacttgagacaattccattgcctcgtgaagtcgccatattatctctatatatatccaactaatcaaattagtatttctgtgtttgtgtttgcatgcatgatgacgcaacacaaacacgtacccagggacataaaatgctgagtggagcacgcaatgatatgaaaaaacaatgtcttaattttttgttgttgagttatgaagaacaatgtctgaatgaaacaaatgaaaaaagaggaggggaggggtcttagaagctttagattaggaatcggaatattcgtcaacgccaagatataatagccttgcattgcatgttgatattttcgtcaaagcatgagagttaatggtgttacgggggcactaatctaatattatagccactcgtgtaactttaaaaaaatttctattttggcaaaatataatatctatatatggattgagaaattctacttaaaactagcgtaccactaaaattaatctcatacgaagtcaacacttgaaagtttaaagtaactaattaaaggttgccctaacaatcataggccataagcccttagtgggccttgtggtgtgggccattttttaaaaagtattgtagggacaaaaataaccttaatgaaagatgggagtgaatgacttttacacccttgctcatcttcatgtttttccatagcttccaatggtgttggacttttgtggtaattctatttggtgttggtcagggaatatggaggcaagtgttcttcttggaaacagttatggggtggtagctcgcgtggctgtggcgggtcttcgtggtggagtgatgggttcggccatatctcgtggggctaaagaacaaaaacaaaaataaagctctgatgggttggaaaggtgtcaaatacactcagcatatttaataaattattattaaaaaaaataccaacaaagttgcaagtaacatatcctaactcttaattaataaaatgcacaatctaacagtttatatttcctttatcactatgaactatctaacaagattcaccatcttaaacttcatcatttgcacgaacaaaagtatttaatttattttatctcataaatttctatgaacctatttttggagatgttatatggtgaacaaaaaatattactaattcactaagtttatgtgcaattaatttttagtggagtcaatttatctttaaaatgagtcaaaggacctattctatggcaatcaacccttttcattagcttctattgttctaatatacaaattttaaatcctagtttttggtctcaaatgtttttaattaaatttgttaaattattttttatttaaccttactaatctaaacatcatggttatatataaatgcaagcatatttattaagataatattttaaaaaaaattatataaatgaaaatgatataagatgtatatgaatatataattatttaaatatatatatcagcattattaattttttatatttttaatattaatataaatatatttttataaaaataaaaataaattacaaatcgtaataatatttttgtaagaaaaataataaagataataaaatttacaaatattatatttttaaaaaattgcataaattatttaatacatgttataatattttaaatattcatcacgtttacaaatattttcatcatgtatactaatatatcttatttatatatttaatcaaaatattaaagatgtaaaaaaagatataatcttgttttcatgtgaccgatattttatttcatgtaccatttatcttttagttaattttttataatttaaaaaattaaataatgtataaaaactaaaaaaaaaagtctcagtaaatccggacttaggataactaaaacaagtttttttagtcttttaagtaaatttaaaaaaaatatggttaaataaattctattaaaggtgaccaaatagactttttagcatagattaattattaaaagttgttaaattctcacaccaataatataatgacaaaaaatgttaaattatacttttagttctttcgtatttttaagatttattttgtttttttatctgtcaaaaatcattttaattatttatcttttaaatttgggttaatttgatcctttgaaaacattgatcatttttaaatttgagatataattttgagtattttgtagtcgattcaagatgtaaaatttgtgttactaagtttatctacataaaaattatgaaaatcaattgatgtttgaataaaaaaagactataaacatgataaaaacaacatgaaaagtgaatctaaataaataaaaaaatctaatgtcaaattgtttttttgtttgcttctaagactcactcttttacatattttccatcttatttatgactcacttttattcaaacaccggtcattctttatttagtttttaagtagataaatttaattgaagaaatcccagctcttcaatcaataaaaaaaaggatttaaaaattgtgttccaatttttttttaaaattatcactgttttagaagaaccaaattgacccatatttaaaaagtaaatgaccaaaataaatataaaaaataagttaaaggaccaaaagtttaatttagtaaaaaaaatataattattgacgtgcttgatcattattaatatgtttatgtgttatttaaattatagaattggaaaaatatattatatattaattgacttgtttttaattttaattgaaacaattatattgttgggtttttgtgacttagcaatcctattatattataaaaatagttttataatttactttattgtcaacttcaattaaaacaatatgtttctttgacttattgttttaatttcatcaagtatataattgacattaatttacttattattgatttgtattgaatctgatacgtttataaaaagaaatttgcagtacttattttattagtgattaatatcatttaatgtatatatattattaatttgattgatattaatatttgtttagttgatattgtttcttaaaatgacatttatttataacttcgcataaaaaatttaaatttcaaaatgatagtcaatataaacgtgttttatgaaaattgagataagatatccttaatttaggaaactttcttattatatgacttaattgatttgattttaaatgaaaaaataaaataaatattttttaaaacatatatttatactaaaacaaaactagagttatcttctatgtaaatatattttttattatatgttgttagatgaatttgaaaaacacataattatgaatacatagtatgattttaatcctagttaatatatttataagatgaattttaatttgtatatctgccctccatcattaaatcatgggtctaaccctgcttttataagtactcttggagcttgagccctataaccctaatgagtatttgggacccaatgacctaaatattttttttcttctgaataaacattttattcattaaccaaaaaaaatattggtttgagtatctgatcttgcactttaattgcaaatgtccactatggtcaatctctcaaatgagtacgcacaaaatttaaggttttttgaacttcaactctcactctacgatttactccacgcgcggtctacttcatttttatttttccactctcttttctttggtgcagcattgcttgttgcgccttgagttttaagagtatatctcaatagcttatttcaggtaaagattgactccagtttagggcgccgtgcaacatgtttatcggagtttgataggaattttctgttaggctacatgcatatggtagatcaagtttgttatgcatttgaagtttgaattcatgtttacgtttgaagtttgagtacttaatttttgccacgtatagaaagagaccaatggattttgaattcaccatgaatactattttttaaaaagcaatcggtttttcctttgttaatggcacttttttgaacatatataggtgtggttagttatacgaacaaaaaaaaatgattgaaaaaatcaatgctgcttgagttgttacacaatatataaagttacaacgtttccatagtttgcagaaatatgtttggaagagtaaatgttacaattattccattctatcatcattcttaatttttctctcactttattgtggtaaaggggtattgtatcccaacataaacataagttgtggatataaaatcaggtatatttacatgtcagggagattgcattgaaacttccaaaaatatgcatgatatgatttattatatccatgtatatatgtcaagaattagccagcttatcgtgtagacttaattaagccttgatcttgaatttgtattttatagtagttgctgcctgatattacatagtactgcaggtgcacgcaatacaatgatgcatagaaaaagtcatttacgtcaacggaagcatccattccatgcttgtcgtaagtctttccccttcttttcgttaatacatcatcaccatgcatggcaaaatagaaaataaagatcaatggattagactttagagcttatattgatgattttatgatattggagagattgttaatgccctgatccacgtgaaaggacatctgtaattcacagggcatacttcagtgactagttctttcatattatgtgtacccaattggctacaatttttttttcctataatcatacggtgctcacattcattagttttcattttcgtactatattaactccctcaaaattttgtttgaaacagccttcgaggcatatttgtctcggtcatggcgagctgtggagctcataaaatttgagtctggaactacgaccctatattttgtagataatcaccatatgaccattaagaaaggctccttttcagacgttcgagttaggtcaaggaaagctactttatcagattgctcctttttacgaactgggattgacatatgtgttctctcagcctctcagggtaatgacaattcagatgaatctagtgctaatcatgtaagttaatgccttttagttgtagttctatataaaattggatttttttgtctatctcagttactctttatcaatttttctagcgtgtgtttgattaatcttcatggatgtggtagatttagtttgtattcttccttgctgttgattgcttgatacttattagatttttggattattatgacattattgtgaagtatccctaattttgttgataactttcttaaattatttggttggatatcttccctgaaatctctctctcaacaacatttttttgtctgcaagactcaaactcaaaattttccttaaggatttgagtctagtgttactcgatctatcggtagatacttattaattctctttatatgagatgatagccaaacaaaacattaagagaaattaagagcagactctctaacatactcttctctaaacaaactgaattatcttgatttattaatctaaaggattagtttttgtatgcatttgttaggtgattattcatttggcctttcttgatgttgtttttagcaccagtagacctgagttaattctctcctttaattcaagaattgtagtctttaggacattaaaaatctgatcaagtgtttctttctctttttgattttaggtgtggcttgatgctaaaataaattccatacagagaaaaccacataatccagagtgctcatgtcagtattatgtaaacttctatgttaatcaaggttcacttggtacagagctgagaactcttaggaaggaggttaaagtagttggaataaatgaaattgccatcctccaaaagcttgaacgtaatacttgtcaacacaaatactatcgatgggaatcatctgaagactgctccaaagtgccacatactaaattgttaggaaaatttatatctgacctttcatggttggttgttgcatctgctataaggaaggtttcattctgtgcaagatctgtggaaaacaatattgtgtatcaaattttagggagtgatgctacaacctcttcattatacatggattctgaaataagtgttgtgaactttaaagtgaacgaagacggcatgcaaatgcctgttattcatctagttgatttatttgagactgacaccaatacaagcggcgataaacatgattcccactatgatgaagtgccatcatcttatggttttgagggcttacgacgatccaaacgtaggaacatacaacctgaacgttactctgattgtggtaatgtttctgagataaaggttggtaatgttcgaacctggccatacaagttaaacaaaaggaaagatgatgatggtggtggtgaagagtcattgccattagcacaagagaatagtgacaatagtcaaaaggtcaatgaactgagttcttgccgggagattatagtgtaccatgggaggaatgaaacgctggaattaaagtcaggtgaggccaatcaaactcaacttgctagtgttcctcttcttcaagaaggtgattcattagcccttgagcatcatcatctcaatgacaatgttactagaagaagtgatgcatattatagcacccctaagcttaagaggaagagattagttgatctggaagctgatgtagattttgatcctggaagggaaggcataaattccaataaaggagttagcgagaaaagacatggttcatcatggtattcaagaagcagaagccatgctgcagaacacagttataaagacagaagcttaaatgcaactgcctacaaggaaatgatagattcatacttgaaggatgtcaatagaacaccaactacagaagagccacctgtaatggaccagcggaaggaaataggcaactttgggcaaaagaaggaagcagaaatacctgaaagagaggacgaggaacaaatctctgagatcgatatgttgtggagagaaatggaaatggcactggcatcaagttatcttgaagaaacagaggtgtaacaactgattcccttttctatgttgcatttcttttacggagaaaatttagatgcagttccttaaatattgttggtgttgttgttcaatcaaaattggagttttacttagttaatctgcataacacaagtttgcgttaaatgttaacacatattatcaagataaaacttcaattctaattagagaacaacaccaattaataccgaagaaattgccaccaagttttgtccttttatttatatctgtatattctggcttttttatcttcttttctgaggttatttcggtgtaactatctcatcagggttcaaatagtgccaattttgccaagactacggaagaatctaatcgcacttgtccgcatgattacagattgtctgaagaaattggaatttattgctacaaatgtggctttgtgaaaaccgagataaaatatattacgccacccttcgtaagtcaagttcaaaaccatgtttggtttgatttctttaattcacttttcaaaaagcctatgcaactataaacatagttcctcatattgactataacctcccaatttgttcaaaaacctgttcatattggcagattgaaatgcaacgctcagtgaggcaccaagaggaaaagcaatgcaatggaaaagatacaaaggaaaaggctagtaaagatgatgatttccatctgctctcaactcatgctcctacagatgaacataactctatggaacatgataacgtttggaagttaattccccaatttagagaaaagttgcatgaccaccaaaagaaggcttttgaatttctttggcaaaatattggagggtctatggagccaaaacttatggatgcagaatccaaaagaagagggggttgtgtgatatctcatgctcctggagctggtaaaacttttctcatcattgcatttctcgttagctatttaaagctattcccagggaagaagcctcttatccttgctccaaaaggcacactttacacttggtgcaaagaattcaacaagtgggaaatttctatgccagtgtatctgattcatgggcgtggtggaactcagaaagatactgagcaaaattcaattgttcttcctggttttccaaatccaaataaatatgtcaagcatgttttggactgcttgcaaaagataaaactgtggcaagagaaaccaagtgttttggtcatgagctatactgcatttttagcattaatgagagagggttcagagtttgcacacagaaaatatatggctaaagcattgagggaaggtcctgggatcttgatacttgatgaagggcacaatccaagaagcaccaagtctaggttgaggaaagggttgatgaaactgaaaacagatctaagaatactactttccggtacattatttcagaacaatttttgtgaatacttcaacacactttgcttggcaagaccaaagtttatctccgaagtgcttgatacattagacccgattaccagaaggaaaagcaaaacagtagaaaaggcaggtcatttgctagaatcacgagctagaaaattgttcttagataaaattgctaagaaaattgactcgggtattggaaatgagaggatgcagggtctaaacatgttgagagaaaccacaaatggttttgtagatgtttatgagagtgaaaattttgatagtgctcctggtttacaaatctacacgttgctaatgaatacaactgacaagcagcgtgagattttgccaaaactacacacgagagtggacgagtgcaatggttaccctctagagctagagcttttggtaactcttggatcaatacatccatggttggttaaaacaacctcatgcgcaaataagtttttcactgcagaccaattgaagcagctagacaaatacaagtatgatatgaaagcaggatcaaaagttaaatttgttctgagccttgttttccgtgttatgcagagagagaaagtacttatcttctgccacaaccttgcacctgtgaagttattgatagagttatttgagatgttcttcaaatggaaaaaagatagagaaattctgctgcttagtggggaactagacctctttgaacgcgggaaagtgatagataagtttgaggagcatggaggagcatcaaaggtactccttgcttcaattacagcttgtgctgaaggcattagtttaacagcagcttctagagtgatttttttggactcagaatggaatccatcgaaaacaaaacaggctattgcacgggcttttcgtcctggtcaagaaaaaatggtttacgtttatcagctcttggtaacaggcacattggaggaagataagtacaaaagaaccacttggaaagagtgggtttctagcatgatttttagtgaggcttttgaggagaacctttcacattcgcgagcagtgaacattgaagatgatatactgagggaaatggttgaggaggacaagtctaaaacaattcatatgattctaaagaatgaaaaggcttcaacaaattgaagagaggtatgaaaacatgtgcataatttatgtttatatgtatcctaatcctacattctccgtattagtgttgttaacagtgtttgcactagatcactagaattcttgtcggcatgtaccttcagtgtttgttcaaaatttccatatatgcatgccactttagagttttgattggaaaaaaaaatccaaacaccacataaaattaggcatggcgtgtcgaagacagatttgactcttctctgctgaaatgcaacgcaaattcgagtttagtagaaacttatcatccaaaattaaaattgaaaactttaatacaaatgcacattttggagccattcatgtcatctcttggtctgagtcttatcattctgtggattgaattcatggtttctcttatgacattgttgccaagtaatactactatataaattcagatttgggtttctgataaccgtggtcgttaatactatatatataataccttgcaggagcttgcgcgatacttgaaacaggagcagggacagtggaaaataaaggagccatagcaccatctgcttgcttatgtaatgtaacccaatctgtctatattttaatacacaccccattacgataaaattatgctagggcctaatttgaattgatttctattttatgggaaattttcaactgaaaaaagtatttgaatttaatttacaagaaagtcataaattataatagttatgttgaatgaaaacatttttaaggagttatttttcaaagagaacattttaaaatataatttgtatgttaaaaaatatattataaattttagttatacgcattgcataaactaaaataattataagtttataaatgttaatggagaagttaaacaaataaattttaagaaagataaatttataaatgtgtagcattgtcctacggattttttcaacaaacacacatagttctccttttttggtaattgataagtgttattgcatatattatttatatattaaaatcatatagtaattatctcatttttttatcttttattatttattgtgtcttaaaaccataagaattaacttttgagtttttatctaaaagatgttaaagttaatgattttagaataattttggttgtattttgtgtagagttgtagcagaagcatgaaagaggattaatgaactgaagtgtcacactcaacacgatctcgcgagtcaaaaccactcaatcaagcaagtcatttagcgcaaggagtcacattgaaagacagttgtcacaagcaaacgcattaagcgcgcatcctgcgcttagtacgtggccacttgatctataagagagttctaattgaccaattaattagtgaaaacatataaaaaggaaaggaaacatttgtttccttaagaatgaagaaaccaaaaagaagtaaagaagaagaagcaagggaaagcaaagaagctaatataaggaaaatccgtttctagagctctagtagccaatctgtttcaatccatttctctttcattttcttccctctcatctcacttttatatttataagtctctcatgataatgaatgactaaaattatctattgttgggagtttttcaaaccaaactctctttagtgtaatgattttaaactatcttttaatataatgttgttattattattcatccctatgcttatttacatatttatgggaaatgtttgtatactaaaaacttatgaagaatatctaaaatgagtcatatctaggatagagtgattttttttagcatgttcatgcatctttgctctgaatgcaaatcatctagtaatcaatcaccaagggattgagagcgatattaagtgatttagatttttttatttgaggaatcttagttagaatagactagtagatgtagataataattatgttaatgttaaatgagaaaaatctattaagattaaatcaagagaagttttggcaagcaagagtcccaacacatttcttaactcatcacaatatcatctcacaactttgagcgtttgtagttgctttgtagttgattccttttaacttatactttatagttgttttgtagttgattccttttaacttatactttataattaatcaatgaattagattggtgaatattagttattgattgttaattttttgttagaaggagatcgaacccataatttttttctctttctattctttcttaattactcaactcattttatatcttcaatttcacgataattaattcttctacgaaaaacgttttcgtaagccttactttataccatactagtttaactcttagaatcctatatttcttcttaaatacctgtttgcatttaattggtttttcatccattcttagatcaaatctccattggtgaacattcaagaatccaaattcaccttgctctcacaccaaaaaaaaagaacatagaggagagaaaaccaaaaagtggtgaaaaaagtgaagaaacacacccatcaattgtcatgaattctaatcaatcccataaatagtcatgcgttcataaaaatattaataatgaaaatagtaatcatatcatcgtgcaatgcattgaaaaaaataggtgagaaagctatatttaaaattgaaaatggagtatacttttgattaaatactaaaaaacatttttaggaacaatataatgaatattgtatttaaaaaaacattccatttgattaaaaaaattgattaccataatatataaaattaaaatttatataatacttaattgattcatttactcaaagtatatattcgttgtaatcatgataattagtataattcggtatttttatgagttaaaaaaagaaagctgtaaaaatgatcagttataaacgatatataagacatgaatattttgataaaaaaaatgataaattgtatggcataaacttgattattttgagtgttttaagatgtgaaatttttagttatactttgtcttgtttttttatatcttttaattgaattaaaaattaaaaacttttctcataccgataagtcataccaatttagggcaaaaacttttctcacaattttaattattattcttttttatattttttaattttattttaattaaaagtgttgtacgatgtacttaactttttttttatataaccccctcatgtcaagttggagaattggattatccatccaacttgatacaggcatacgttccagacctaaaatgaaataataatattaaaaaaaacttgattcagaattatcgatcaattttctttctgatataactaactatatctacaacaattatgttttagtgatgtgtctcaacttggctgttgcttaaaattttctgattaattatctgttttatattactcatattggtatataaaaagtgattatcaccattaaattattttttttttctagtggatacagtgctataccgtgtatctggtattgctttaatttttatagtcgtatatcttgtatcgttatatctcattatattgcgccgagtaattaattaacttagccagagaatatttatatattataaatgagattcctcgaatttgatcaaagcttgattagtcttgtatgtcggtataaataattcaagaaaacaaatatcaagacaggacaaaatcataaaataacaatattgtcactcttttcggattttttttagtgattgaacaaaaaaaattcaaacaaaaacatttcgttccttttaaattatgaacactttaaatttggagtttggatagtaaaatattttaaaacgaattttcactccgtataataaaggactcattttacaacatcaaacaaacaaatatttaaattgaatttttattagagtttaatacctatatatgtaatatcaaagatggtgaattatagttgaatgatcatataaatttttttacataattagtatataattttttttcttttttataaacttatattttttaataaattttatatgtaatgaatttttatcaatttaattattaaattgaaaattttcatgaaattataaacacacattatatagtaatttgacacaaatgattaatgtattaaagttaatgaaacacattacatacagagataggagataggaaggatttaatttgtattattttaataatgtaagtcaaaattatttttacacttttaaataactttttactaaacaattttattataaaaaatattagattgaaaattcctattatacagattatgtttataaaaaatttatcactttaaacatgtatatgtgcatgttggatatacatatagaagatgactaaagataagatgaggtgctcgtcaaaacttctacaaaagaattggtcaaaatattttgagtcagtgaatatgctagtcacaaccctcttaacttgattttaaaaataaaataaaataaaaaacctcttaacttgcttcaaaatgaaacctcttgcattaatccaatcgtgcattgaatgagtataaaatagtctacagtggttagcaacagtctcaactctcaaaaacttgaaccaagttgtattaattaaaaaatatatactgtattctataactgaaaatatcaattggcaataatttaggagcagccgctcccacattcatttactagacagctactattttccttcctctatatttgaatttgaattcttttaaaaaaattgtttttcttctttataagactctcttcaaatattatttcttacgttaattttcttatcaaaatatttttaattattttaaaattttttagtcaataaataataattattataaattaataaaaacaaatcttttttctctcttataaggattgagaaagatgaccagtataaactaataacagaaactaaataattattgttctttcttcatacattaattagttaaatgaacaataattaaatgaaaaaaattgagatgttgagtctcaataattttaaaagtaatttggaaaaaataatgtaaattgttaataaacttaatgttattaattcaattaattaatttttttattcttgttaattggttaaaagatttttgtgtataaagatgaaagaagtaggtattttatcatcatccaaggttatttgattatttttcacttgtgttttattttaatttaaagggtagacggacaatacgggatcgatgaaggttaattattgagttaaaaggaaaaagaaattcaagttggtggaagttggtgagctttgggggggaaagttacgaaagggacgaagaataaagttcatgagaaggaacgaatcattagaaaagtttcaagagtaaaataaacggtaaaactaaaaccagtagcgaaggagataaaaatccataagctaataatatatgcctagttgatagatgaaattagggagaaattcacaggttagaaataggtcagatgtcttgttagtggtttgcatgtttggctcgcattaaatttaataatatctaaaaaacattgatgataataatatctaaatttacactaaataagctaagttaaaattattttaaggcttatttaatgattatactacaaaggttttaaatcatttaagaaatctttgactacaaaaaattagcttatttaaatatataacataataaaatataatatatatacatatatatatttattatgttatattatattatatttttactctaatcttaaaattatatatatatgcatgcgcgtgcatgagttaattgaagtgggattaatataacttaattagtgacctcgattctagatcataaatatgcaggtatattaaatattagaataaaaaaattgttgtttataataattgtatatatgtcgctagcaagattgcttttttaaaaaaaatgcatgtaatttgctatttcaaaaatttaaaaatgacatgtgatcaatatacattattttttaaaataaaaaaacttctttttattaatgattaaattgtctaaaattatgattatacatttattatttgtatacttttattgactatgttttatggttttatgtgttaagctttggtgtatataattaaaatgagtttaatatttatgtattaatagtataaaatttatcatacatgatgaatggtgaaattttgaattatgattaaataattatataaaaaaatttacatgatgaatgaataactttttttttctcaattaaaattatgatcctttgtcgatatgttttactgtgtcgaccttttttttcgggggagaggggaccagtaggagaagtagtatttagtaaaagaagggagagagaagttgacttatcctttaattagtttagagaaaattagacgagaaggaaaaaaaataggcgaaagtcactttttctttctatctctaccaagaatgttgatgaaaaagtggggagcagaattttaaatttttattttcatatttatccttctccacatttttgttttcttccatttttttataaaatgatttattttagggcatagttaacttttcaatttttttcatttctattcgatcaaataaatagaaaaataattttacttttctttcttttaacctttttcatatttctctcataacgaacaacttattaatttacctcttttcccaccactttttgtctatccaaattctatctttgaattttcttccttttcattttgtttctcaaaccaaataaagaagatcgagtttggataaatcataaagttatatacctataaatagaagaacattaaatgatcaaaggacataaaattaattaattaaattttgacataatttaaaataaatttataaatctcaatttttttctataaatcatttaacttttttataaatacttataaacttaataaaaattaatatttttgtatatataaaattcttaacattgtaaatttataattaaaaaatctataagtgaaaagctaaaaaagagttgggcctagctaggcattataattaagataacgatttaactaataattcattcgataagagttgcttttgttatatataggtgcttttaaataagtttacattgatagattaaggtaacaaaaatgacttttggtatcgactcatataatttatttactttattttaatatcttttatatacaatttatcagaataattacacggtttttaaaatgaaataagctcaaataaattttctagaaggcttttacagacatcgatccccaagtatgtgtttggctttacatttgaaaaattccaaactatgattattggcaaatttgttttttgtacgaaacgtttgtttaaataatgatctggagattacaatgaaacactaaacatattataatttgataaattattaggtgacgtaagcagagttagatttcagttctgtatgctcctcacatgcctctaatatctcaattgtttcttatatataaattgtaagaggctgacacagaagattttctgatcagtcatcaaataattgaactctaaatatattgctcgttatcatatatgtaaaattttatcttgcctatgcttgttaattttgtactctcgaacatgaatttggaaacttaattagttcataagataataatgcatatcaacccgaatcattcacacatcaaagcaatgttcacttcaatgggaatataaattctttaaaatcatccactagtaatacacctaaatgctactagtaatatagttgtgacaccatgcatgtttgatttttagcccaatttcaatttgttggcgtagctttgaaaattcctaaacagaacagtaagatgatccatggtgcatggtactgagataagtaaaataaatctttttgagaattgatttatcttttcaaaggtttagaattttattatggggcgattaatttctaattagcacctttgactgtctcttttgcgtagacaaatctgctattacgtaataggtatatccattttattcaatcgttattatatcaataatatatattattatgtagacatcaatggatcggaatattttaagaggcattcaatggtcaatttatgtttttaatttgtttcttttttttatactaaattaggtttcctccctagctaagcatctctttgaaaaattcaaaaatagatatatattgaattaaattgattaaaagctgagtatttcagttattattatgtatgatttatcacttttctatctacccaaaaggtttattagtttatggtttctgcaataaaacatattttaatttgttacctttcagtctaacatattctataatgggtttcgccatcacacgtgaacttgcttcttacttcagaattttgctatgtctgtgaaggatcc

SEQ ID NO: 5 - DNA, 2915 bp, Glycine max
tgcatatttattttacaagatattttctctaactctcaaatatcctcctgcagttccaattataaattactcttatttcagtttccttttaccaaaattgaagttcaattaataaaccaaagagactggtatatgttcaatcacatgcgataaaaatgttccacgttcttgttccgagcagattctttgtaatttcataaagttagagaaaagaaaaaaaaaacagacatttagtcgccaatgcctaaaaccatataataactccacagtttggttctctgaatgaattcccttcattttaatccaaatctcaactacctcttcaattctaaacaaataaattagaacactaccaagtgatcctctggggtcttgacatgagccttctagttctagcttttcaatcaatgtatctacagagcatgtattccattttggattatagagaaataatgtaaaacttttaaccaaatgttaccgcaaatctaaagaaagttcattgctccatgataaattgataatactacataagatgtacaactgctgattttatatatcattttaacaagacttgccaagagatatatcccttaaagccaagagcacttatgtttcgatttgagacacctctatttattccacttacatttgaaaaataaaaatattacattcaccaaactgggaaatgggaaatatcaaaacgtatagaagtgagccgtggaaggaattgtaaacaaattatgacgaaaaccagaacctattcctttgtgcctattttaccagcttttcaagaatagtacaaaattaccaagaaaaaaaaaatgatgcaacgtattttcaccgcatttatttttctctctcatcaatgctggcttcttctgtttcgttgcataaaattgcagaaggattcggaggatctgatgctgatggctctgtctttctaacttgtaggccagtaaagcgcgctaggtcaatccattgctgcagcaactcacgaaaacaaaaaaatttaaaactcctagtcaaatttagaaatgtatgtgcagatgaataattcacataaaactaaattataccccattggttttaaaacaattgctactcttgattcttgaggttttagtctgtttcgaattatgtcactttagaacatgaagacgacattgcttttttctttgtcttttattgtttctctaactaaaaatgaaagcaataatggtggaagaggataatatagtcaaacgaacttaggtttccttgaaattaagaatatttaatgactttattaatcatagtagaaaacattaaacaattattgtgaaatggaagggaagtatattgcaatatggcaaaagcaaatgctaaaacagtgtctttacaatattggctaaaaagtcaaaagaaaaaagttattagaaaatgtcactaatacatttccattgtgattttcaatacatccctaatgtaatttctaataaaaaaaaaattctattattaattcctcaggaccttattacaaatgctaatgagtttccttaagactcttgtcaagggattaaaaaaataaaatatattaaatgaaaagtaatgtattacatgttgtacttttcataaaatgctctatatttttggcttaattatacttttgatcttcttactttttcaattttgtaaactttatccctctatttttttcccacaattttgatctctaattattttaattatccattaacttaacatcatccaatattcgataaatgtgctgacatggcagtgtagaagagtgtcatgtcaacatgaacgtgttgacatgctagcaacacgttaacaagaccctttttttattccttaacaagtaccgtaatggcactcgttagcaacacctttgtaatattaagatcacacgttataagacaagcggaagttattttctttccttaacttgtgatttattgttcctagacaaaagcacaagctgacgcaattataactcctcaaaacacatatttccatattaacacttgaatgtgaaattcaccactttaaaaagaaggaaaaattaaattagatttttgagaagaattatagtgttcaaccataaataaatgaaatccacttactgacgagattgtgaacattgagcattgaaagtgaacaaaagcagatagaagataaggaaaaaaaaactgacctgggttccccaatatgaattgactgtccgtgctacaaaagaaagcatatttacaaagaatgtttgagaagcaaactgttcagaaatccgatgctccactatcccagcaattatctgctcaacaaaaatattataatttccgtattaatacaaaataacagttccaggcattaaatggatgtatatttgttgaatatgatataaccaaagatgtagtcacagaacaatcaaaaatcaattttaagaaagaaagagccttaaaaagttattcagcaaagtgcagatgaagaagtagaaaggaggataaaattacaaaatcagatataatagatattattgaaccgagtaattttttttccacgattacttttaatagcatccagggtttactaacataatatttggttggaaaataatggagaggaaaggagggcaaagatttggaaggaaagggagaatggaggggagtaaaacacctcctctatcaattttggctcccttccaaaattgggagaatttggagaggagaaagttttacatgaattggactaaactatccttaatggttttatcctattatgaggatataataaataataaattattttattttctctcttatttccttgtgaaccaaaccaagtgttcctcccctctactccctttctttgaacaaaatagatagtatacgatgtagtccaatctttcctattatgcttgc

SEQ ID NO: 6 - DNA, 2942 bp, Glycine max
caatgacatgttttgagagatagacaaatattccctctaacataacataaaacacacacacacacatatatatatatatatatatatatatatatatatatatatatatatatatatatatatatatatttgtggtagaaagaaatattagttttagttcgacatattataagatattagtttctttatgtgaaattcatgttcttttattatgtgtttaatttaaaagataaaattatataatctaaaaataaaaaagaaatttttgaattaaaataaaatataagaggtgtaggattcatattattttttcctcaaccaaaaagacaccctaaaaaagttaccaatatactttttaacatgttattttaaaagtatttttattaacttaaatttattaaaaattatatttttttttaatttcatcttttattcaatgaattttccctataatttgattgttttcaataaattataatcattattaaaaaatatataataaagaaagtgtatcaagaaaatatatcaagaggttattcttatttttgcctctcataaaatcctagacttaaaatatacactctactttttgtgttcaataataaattcgtatatttcgtggtcgcaaaaaaattaaatgatattgataatttacatagtcacgcaaactatatatagaaggcctcaaatttagagctctacgtgaagcattggtgtccatagagttacatggtgccaattaccatatctttcattattttggaattttcatgtaagataaaatcgatccgagtcagatgtgactcaggtctgaaacacaggtgccgatccaatatccatgtatgccaataatacgaggcgtctactctaatttgattgaaaataagggggcaaaaagtaaaatatatactgccaaattccaattcaattcaactatacgacattgtctaaaagttagaccaaattgaccaactgaagtgatacctctttgttgatataaaaaactcgtgtgtgctatgaatatttttaaaacaaaataatatttatgagataaaaatatcattattgtcaaataatgatgaaatactatcatgtaatttaagaagaaaaaatatataatagaagatgactttattgttaattatgtgaattctattttattatataaaataaattaaggttatgtttaaaaaaattagttgaaagttaaaaaacaaactaattgataaccaaaaacttttaagttaatttattaaattataaatatttgatagaattgttgttgaagtagataaaaaatataatatcacaaaaatagatatatttatatgatatttatataaactttaatgttttatggacaaaagtatattgaggtattataattttattttttaattaattttaaactcttgtaaattatttttcattatatcttttgtttcaattattagattttttcatgttacatattctattattaatcttgtacaatgtcttaatatttttaaccaagtttgaaataaagttgaaaaaacatgcagtaaatattatacttttattatattaattagtataatagttaaaataaattgtataatataaaaatattcagaaaataataaattattttaacattttttactgtcaatttcgtgaagatgttgaaataattacaatggctaagacaaatagtataatataaaaaaattgtaagaggaatgagtgaaaaataaataaataataaaattatgttatatttaaaaggaataataagaatatttaataaatattttaagaattaaaaaataaaatataaaagcaaaaaattagaggctaaaaactagagttttaaaaaagttactttaaataatgtttcacaaaataataaaagttacaaaaaaatacttatttattaaataattaaacaagtttttcaactaataaaaaaataaaactaacttaaataacgtatcaaacatagcctaaagcgaattttaaaaaaaaaattaaggtggaaaagcatcaaaattcaaagttggtatcaaaattaggattaactaaatttaagcaataatatatgtatccttttcctctcggccccaaagttcatgatcaatcttatcaaacctttttaacttatcaattttgctttcatgcaaaatctagtaagagtaacttcaaattaaatccacgtttgatagtgagactcaagtttaaaatcatatctatcttgctatatgtaataatcatgtttgactgctgagtttgatgggtcaagactttcctaataaaataaaatgtgggtacggatgcttagttttgatgggtgcaaatacaattggaaaaggtatgcatcattaacaatgttttacacgtctaatttctccccctctgattctcaagaggcagggacaaacagatttcacatgcccttttctgggatacaaacatggtttctcactttctcatggtttagccttaaaacatgattcatcgcaatctgcccttaccatttgggaatgtgactgaatagtttggtaactcagaatttgctacaatctggtgataagtaatgactattagatcataattttgttgagaatacaatatttctatatattctaatactacagtgtttctatttctggtttcaaatctcaaaaacaagttatacacaaattcttttgggagaaaataaataataataaaaaggcaagctagcaatcaaactccgcaactaaagatacataacgaggtggtcacagaatagcttatccagtacaatttaagaaattggtatacaaagtatgattttcaacaacgaacctcctttacccaatatttagtcacatttatttgtaacctattaaaaacttttgcgaatagctcccctaataaaaaatgccgcatgattaatcatcaacaggaaaaggctagct

SEQ ID NO: 7 - DNA, 6231 bp, Glycine max
ggatcgaatgaagcaacttaattaatataactctctctctctatctctctaattcggttgcattcaggtgtggcttcacatttatttgtagactcttacataatgctatgttatgtactgcaattagcaaatactctttctagtggagaaataataattaaaaaagtggactgattggtacgaccattagtttaattagctccatggagaaaagcaagataaaattgctaattattggttaagaaaataattgcaccagatatattatataaaatgtcaaaaacgcattccgtacattataaataatattatatacgtcatatttacatcattttttatccttgtttatctcaaaaaagtgtaaatatagagagagtatatatcatatcatataatatgtaagtttttattagtttaaaaaaatagcttgagagtaatgtgatttgtcatgtgctaataaaatatcattttgaatgctcttttatccacatatattaattgttaatgattgaagtttattattattattataatatccttttaacgatgaaagtttgttttaaaaaaatatagatttaagatgtgtttggaggaatttatttatatcttatctgaacttattttatggcatacgtgtaagtatttaagaaaacttataaaattatagtttatgatttatttataaattgttttcaacttattttaataaaattttcaaaataacttataagaacaaattaaattttttatatgaaaataatttaaccttattttcttttcaattataaaaaacaatttacaaataaaagcttatatatatgatacacacttttaagtgtttaagtaagctatctaaaaaaggccgtacagtgtttctttaatgaactatcgatcgggaatgttatatatggaaatatatatacttgagtgaatataggctcgattactccatagtacagtccaataattattagtaaacgaattatacgtttaatttgtatctatatatcttttgttgataattgatgtaatttcaattttaatttaccaaagagagttagcaccacagcgagcatccgttgcctcattagtcattagtacttatcaccgacatctttttgtttgtaaaaggaccactgattcatttacctacatatataatatacaatatgtatgtatacaaaaatcatagtaaggtttaaatgtaatgcttcatgaataagatattctgtgttacagattaagattcgtgtatgataaaatgtttgttattattagagttaaccggcaatttgttcatattgagtctcattaattaccttcttttcacatgttttgttgacatcgagagtgacgatcctaccgagatagataaggatatatatgataacaaattgagataaaaagctctttgcacagtcaattatgattaagaaaaatatcaaatcagttttacagaccgtagctcattaggcagagataattacatgcacgtaaagaaaaaattattgagtcactaaaattgggatagcgaggaatttgagtaatttgaactaagtcataagtttaaatcgtatcgttaaaaaaaatgtagtttttgttactcttttaaatactagtatttttttttttgaaaggttttaaataccagtattattccactaataacctgcctttatttctttatataaagccttctcttaatgaaaatagaatactaattaaataatcgagagaaaaaagatacaaatggagaacaaattatcatgaaaaagttacacattagaaaatatacatgttttagcattgaaaaatacaatggtcaattataaaccaaagaggcccttagttagttagtctaatgtttaagccaccaaatttttggttgataacgtttaaaagtaatagctagatggtctctttcaaagaaatttctgtccatattattcaggtttcaaattttgtttgtaagacgaggaattttggatcttgatgataagaacaagacagggtgaataagttcatttaattaagatggaaagtgcgagtttaacttgagttacgtgtaaggtttcataatcaagtgtacatatgtatatgtattagggtagattaatgatattagctatcaaatttaataaaatgtatatttaatattatttttttatcaacagtaaattttgttaatttaacagttgaatttaaagttttcataaaataaattaaaccccacattatttcaaaaagtaattaatactttgttactacactcttaattatatgcataatgcattatattttgtaataaaaactttatatttacacacgtatgaccattggtgaacctacactgtggcaagtacaccctcattttctaacattcacaaataaaagtttttctaaacagaaaattataataaaatcttataattttatattttatttcatttatatttatatatttatgataaattcctatatttatatatttaaacccactcattttactttttataatttattcacattgattcaagttctaaatctacacccatcgagtgcataaatcaactggcatatattttaacttaatcaaaggtcttgagtttaagttttgaatataaaattaccttatatatttaaaggaagagtttgttatccatgatgattccataagactctctaacaaaattacttccaataaaatatacatgtggtttataaaaaaaaaattccatcaaaattttacaaaaacaatacaaaaagaataaaaatatttttttaaaaaattaattcatttattttgaatacattacttacttttatatatatatatcaacagggacatagtaattcaagactattaatgttgttcacccgtgacacatgtcaactcaatattacacaatcattatcaaatttaattttagaaaatttaatattttttcccattagcatatagtcatttttattggaaaatacattgatgaaacatattatactaattaaaggataaacattataatttataaaagcattcaactatatccattaattgtaaagaaaattttcaattgagaatcgaagttaataattatcaaaataattcttgcttttatttatgaaaatatattgtgtgattcttaattattttcataaatatataaaaatgaatatcatcatatattttgaagtaacttaaaatatatttaatcctaaggttctacatgcttgaacaaacgtcttcatcacaaatctttgtagaaaaagtaaataagacactaccaaaaaaaaaaaaaatcaccaccactacaaataaaaaaggtacgcaaaaagagagcttacactattaccaccctacacactgtcttttatccacatattccttctcaatcggtaaaagaaccaatagctatgatagacatccccggccggactcgatattttttcaaatgttccctcaaatcactgttagttttgatgttaaaacaatttgtttcttggttttgctagtgaaccgcttgatttcatatagcaaaataagttcctttttttttttttttgtaggctagaaaaataagttgcagtagataaaaataaagacaaagcattctgatcgctataattgtaaccaatgtgcaatattaaaggggtgtctgagagcatacaatatcattttgtagccttttatacccatttcacttaatttgcccatgttctctgtccactcgtttgatgtcttctaagtaataactatcagtttcattgaccttgtggtcataactcataactaccatccttgagctaacacaaagaataaagagatatttaggaagataaaattgtgcgaaagtaagaaacattcaattgtaatatgcttcaacaatagtatggccaacagtagtggcgaatctaagactctgactaagcagccataaattaaagaagcttatttacaactagtgttatcggagaatgaaaaattgaagaataataagttcagctataataaactcgagggaggaaaaacaaagaaattcatgataaatagatataacttattaaatttaaggggtgtatttgcacaccctgaattatagagattcttatatctttgagaaaataattaaattgggaaaaaagagataatgactgattgagatttgcctcagaattgttcgttttaatattggtacgaatctaatggttttatcctgaaagatgctcacaagtattgagggactaataaattgtttataaactactactaaatgagatgagactttaaggtgtactgaagcaatatcatttaaaaaatgactactcgtatttgtgttgagaaaatttattttcaatgaaaagaaaatatatacatataagataaagtaattaacataacgaaaggaaataaaatgcaacattataaaaactacaactatataaatgatatatacaactcctagcacatgcattggattgtgaattaattaaaatgttgtatggatggtaaaaattcaaaactaaaccccacacaatttagtgacacagaatataattagcgttgttctttttacagaaaacgacgagaacaaaggtgtcaaaggaaaggagatggatgcatgtggtatgagctcatccaattccaaacatgttgtggaccaaaagcgaagtaccatgaacatgatgatcacgacgattcttctcagattttgggaccgctatgatatgaattgcgactacactactaactcttacgaaccggggtcatcataaaaccattaccatttaccactcttttgaacgttaatgtagcctaaatcttatatccagagaaccagaccctgtttacatttcctttttaaaacgtttctgataaatttctcttgctagtgtctcagaacccagttagctccttcctcaccacgtgacacttcagtgaaacttggagatgccagcaggtttatttcagccagggtctttgtctctcagggcaattcattaatttaaaaaataacatttttttatacatattcatcagtgcacgaggaggagggatagtatgtatcacactttttaattcactttctattgttttctgttagttgaaattcaaatatccctcactaatttgagactgaaacatttcaccaaaaaaaaaaattgaggatggaactttcttttttagttgatcataaattttttcttctaaaatatataatgtggatacatattttttgagattgaaacctaacaaatgataaataagactcacttatttagtgagacatacatgaatttcagagaatattttcctatataggttattagcatttcttttaatatttttttttattgtcttgtttttaaaaagttggcattctttttaaaattgacttttttgagatattgaactattttaataataataataaaattaagttatatagtgtattaaaaagaataagataaaatgtgttttaaatttctcaagattttagtcaaaattagtttcagtctcctctattaaaaatgtgttttaattctcatatttttaaagatatggtgaatttcatttttaatcttgaacagttctttaattttgacttaattaaattcaacatatttcagaaacacgggaaccaaaaccaccatttttagaatccaagactaaagatcttaatgacgtaaaacacaatttacccgtgagaatattaaagctagtagtattgcttttcagtgtgtttcctacggcacattgttgtgtgtggaagtggaagctagaaaacaaaggcagcagaagaagtatggtcctacaaagtgtgtagtagtgaagaagaaatagccgttggtggtggagaggcgcgggtttgcaataaaagaacagcgcgccatgatcctataataaaccctgtcaacaaaaacaagtatgcttcatgaatagttactatttacaaggaaaactagccgttactcactttttcttcttttttttttttttgtaacaaattctgaaccctgcatgttcattctctctctctcacgctcgcaacccgcgcgcgcacctacacttcttttatgtcatcacgtgctccttctcactctccctctctctcactacaaaaaccattcttcaacttgcaacacacgcacacacacactcacacacactgtttttttgttccactaaatcaaaacctcttatctcttactctcattacattcattcttttg

SEQ ID NO: 8 - DNA, 9554 bp, Glycine max
cacaaattttgcattgatttttttgtgccaaatgtagtaggcctactatatatatgttctatgattctttcattcttatattatttttgcctgtttgaatgcttgaatttgtacatactcatactacaataaggtgtagttctggttaattttacctctacctcaaagctggggtgtaattctgtttcctccaaggcacataatagttgaaaatagttctcaggagcattcattgtttattctgcaagattctctttcacggctgctatcttctatgcatgccctgcccataaatgcattatgaagaattgtaacggctgtgtttttggacttcttcaaaaagtttatgttattgccaggtgtatatatcaacatgttttaaagattttcaaacaatcaggttttagatgtgggtttgcatgcatgagattggactagtgcgcttgatgtagtataaaatataaattgtccaatcagcaccctctacatgtccaaataatgggccttatgaaacttaattttttaattacaaactacagtaatctttttgaataaagatttacaaattacaacagacatgtgaagtcgtcatctttcattgccaattctttcaagtttactactattattttcctgcaagcattccacattcacatctgataactatgacagcatctcccaagataatgacttccaagttccaacactggctctgtacatttgaactaattttatatcatttatctattgtgattgaaatataaaattgaagtgatgtgaacaatacgaatcacatcttgaattaaaatatctaacaacgggaacaaataagaggcccagaaaaaagggataaataacggataacaagaaagaaagaaaaaaaaacccaacataattccaacttcaaaattcactcaataaaaagtttaacatgtaaatttacttggaaacaaaactcataagcaaagaaagtcaaagtatacataaccaataataataataaaagaaatcagctttatagcattaatttgggatgctctgcttgtatgcaaatgacacaaccttaccctcaagattgcaaaacacagatgagtaacagatgcaatgtgaatcaataaaaagtattgttgcgttgttgatgacacaaccttactcataaaaaatgcattgttgatggctagcattgttgcaaggtattcatacagtttattctgcaacatagagaaaatacaactcatcaacaccaggaaatggttctgttcaaaatcacggattataaaaagttattatctaaatgttacaagcttaagaagatctatcattgtgaaagtctcttatgcacattaatattacaagcttaagaatgctatacaaatgtttgaggttttgatatttaactttttatgatatgctttgatttaatagttgcaaattgccacatttctcatgtcagttactcgtattctcccataaataaataaggcttcttctgtctcaatttattttacttctaaagcaacaatttctttctttctcattttttttgtaccctctgatcagatcgtagtccgatcctcaagccttagcctctacacactttgtgctcgaggctcgatgattgtgtatgcttctgaccggactgtagtctaatctggtggtggatcccaatctgatctgataacctccacagtattgtgcacattataacactgacgtagggttaaccatgtaccgagatccttgggcacagcatcttgatacgctcaattggcctcgattcctgaatatcggagatgggagagtaggcaggtcgcaataaaaggtcataccattaacgtaggatggtaggttaagtaataatacccttgacttgatgctctcaactaggtcaccttgacatacgacaaagccttttctaaaacaattttatacattgaaatttggaaatatgtatgtattgaaattcacaacaatacttttctaactcatatccaaatttaagatcagcctaaaagcccaaattaggggatgcaaagaattctatcgttaaatcaaacacatcagtcaaaaaagaaaacatacataggcacaatcaactgatcaagtataacagtatttcagatattgttagcgactagaaggtttgtatttaatttgcaaaatttaatatgaatactactggttctatttattttataaactggtattagtaatgaaactttaaaatggggaatattttagcaaaaaataaaggttaaaatatgtctatgattcttaatgaatattccaattttatgtttgctttttagtataaaaaaattccgtttttgttccttgataaaaaaggaaatttgtttttggttttaaacactttttttagtccctattaaattacgaatttcctatttattctttgataattttttgtttattattagtcccttcaaaaattactaataataaatattttttaacaggaaataaatacaaaattctctaatttattaggaattaaaaaaatgccaaggacaaaaaaattattattttattaagaattcaatgcaacaaaaaatttacctaaaagcaaataaaaaatttgagtatttcttaggaattaaaaatattttaataaaaataaaataaagatccaaatgatagtgtgataaccgaagaggaatgtctttcaaccactgcctgaccgccaccactgccaacagcctagtatcaaccgaatccacatataccaacaatcttcagacaaacacttctaagttggtgctgaagagacaatatctcatgggtagatcaaattaagagtgctaccaataacaaaatcgggatcatttgactaacaaacagttatgtgcattggatgttctaccatagtacattgctttatgtgaaattcttttaattattcaatattgacatgttcttatatatatatatatatatatatatatatatatatatatacgagggattgtattatctctgaaaaaagattttatcataaaatcataatgatttctcataatgtatctttacattttaaagttagataaataaaattgattttaaattgttagatataattaaaatacataattaatatgacttttaacaaattgatatataaacacttaaaaaaaagtttcatgacgtacggtgtgtattgttggtacaaaaaaaatttatactatcaactaattaaaattattataaataataaaattaataaaaattactataataatctgtaattagattattgtaaaattgttttataatataaatatacagtcttttttctttaagaaaaattgctagaccaagcaatatggaccatgtgctttctgaaaatatataacacaaaaattccattaagtttttttgcacctataagctacatccgctacgtactgcatgtggagcctcatgagtgtgaggatcttccacaggtcactagtttgacatctgaaagctcctcgtgtaaaacgtgaaaacaaataacaagcttggactggtgtacgatttagtgttactagctatcccatgtaataaatatataaatcttgaatcacaaggaatgatgcaatatatggttcctctaatagtaagttatcccaccaaatctgaatataattaagaagttgtattcgtctgaatgttgtgtctaaaagggttgattgatgaatgatggctacatgtgagagtttgataacaacagctagctagccattagccaagccactaactagacattagttttggttggttgtcagacaaaccgttagacctgagaacgaaagcgtattaaacaaaagatgatatgtagacttttaatataaaaagagatggagaaaccaaattgagatttgataggtgaactataaatcatgacagtgcattagacaagttggtagagtttgttactaactcatcagattcttaagaaaggcaaaaatagaaactacaccacatgtcgctagcgataacgtgcaatttataaataaataatggcttcattttcatggttagttataaattaatgggtcacaattcttaatttattaggaacgtatacttcattttgagagtgtataaagttggaagaagaaaagggatatagaaagaataaaaaaatggatttatctaattcatcgtaaatgaaaatgagattaaatcattcaatcttcattgaataatagaatttaaaaaattgtcttattctgaattgtatcattaatattataactatcatatttaatgtattatctttcttatcatttatgtataaaattaaaaatttataattaaaattatattaaaatacataaatatatgtaatagaattataaaaattaaaattacagctatatataatttcctgtcacttagacttgcagtagacaattgtttgtagttaaaaaatatgaactgtagtccggctacgactattaatttcaagacttatttaacagttaataataattttttgtcatctcgtccccatttgtcttgcgtgttaatatatgttataaatagacattgatatatttttttatgtgtatttgttgatacataacaacaataggattgtaattcagattttaatattttattacatgtttatgtattttactataattttaattaacaattataaagttttaattttatacagagaaattaaaaatgatacattaaatgtaataatcataatattaatggacagaattcataattagacaaatgtttaaattgtattggttattgaagatgagattttttattttcatttttacttggggtcttagacaaacctaaaaaaaagagaaaataacacatgtaataattaagtgaagtaaatataatggaaaaagatgacgaaattaagatgagaagaaaatgctatgagattgaaaagatattaagtgtctctttatataaaactcaattaattacgagttaactggtcatgcatgaaagtgtaaaacatttttatgttatcatttaattataaattatagttaatataatttttaaaataattatcataaaaattaataaatttactgtacaagatgaattttaattaaatagtgatataatttttttttacactaacaagtgtataattctttatctctttaattattatctaacttttgattccacggcatgtcaatattttctctctgaccaaaataactatcaaggttagtaaacgaatataaagacaaatccatcatgttcttttgtgtcaaaatgaggccttcttaaagatcacgctcaatgatatgtagttttctaagtcgctaaaatgcatgttaccctcatgaagctataatagggttcagagatagctttagaagttcaatagagcatgtggacctgggagtgaggtcgtatgtcgctataatgctgtataaacttttggtgagcatgcatgaccattttactactggggcttccatagtgggtttcagtgatagtcttcataagttcattagtctttacaagttcaatagagagaatatatggggacctcggagtgaggaggttgaaagtcactataatgctgtataaactttggtgtgtatatgcaccatctgatggccatccaatgtcccctagggacaacagggtacctaattaattggtaccacaacggggagaaaatcaacacgtttgtggaatatacatacctagaattgaagggctagctcaatcaagctaaacttgaattcaactatagaaattaaattaaattgaaatttggttacacgagtcaggaccattagttataattaaaatgcgttagcacaatttctaacgctataggcatagaagcactaatggtgacacacactagtataaaaatacttttaatatcagttattttagatttttttgtttgtgtaagtcaatcaattttaaaagttacttctaaatcaactttaacaaaaactaatgtagaaatgatctagaaaactttttttttaagttctaactctttttcatcaatgttatacatatatatatatatatatatatatatatatatatatatatatatatatatatcccaaaataaccaatcaaataaactacttagtttacttatatgttaaatcatcgacctattacaagagaggaggtctaactcagatgatttattataatgttaagttattttaaatcttttaatatttttatttgatttctatgaataaaaaaaaaatcaacttacccaactttaaagtctcaagtcattggatattatcttttttaaatatatatatatatatatatatatatatatatatatatatatatataaattgaacatttgttgttatattagctaatattgaacatttatttatatcttaaataacatattatctatcaaaataaaatgttataacaaataataattcatttttatttgtatgaattttcataaatttatttattttcaaaaactttgaactcaatgattgtatgaaataatttatattcttaattaatgaattgatcgtatcctatatatgcatagtatatgaaaaatcaattctcttaaagtaacataaagaggccctttcgtttaagtaattgaattacttagacttcaaaaaaaacaattgaggggaatcaaacaattaacagataattaattctagcaaactatattctcctatatattgtcaaatatttgaaaattaaaactgtaagttatgataatgatattatagttgaacacgtggcagtatggtgacccaatgttcgtcaaaaatcaaaatgatgtgtgcctggtacagtcaagtacgtatctttgagtatgacattcttgtgcatgtaattctttgctgatcttatccatgtgtaaaataaaattagttatgttggatgacaggccacctagtttaatacttgagaaatatttttcataagtatctctaactaaactcttgttagatgcaatcaaatcactttagttacttatagtactgttattttatttgctcacgcatcatggaagtctacggtacttatagtactgttattttattagcctaattatccatgtataagaatcattgaataaatagttagttttaccagatagaaaataaaagagggtaaggaacacccaacctatcatgagagctaaagcttcacaacaagcaacgaacagcttttaaccttaaactaggctaatgccaatattaaagaagaaataattaaaattgtaaggctggtcgtgtataaattaaacaaaaggccctctattcaaaccttcatatatcatacctgtttttaattaacgcggactactttttcatataaaaaaaagatcattagaggattaatttaaagcgttttagtttttaattaccaaagagtataattattattaggcgctttgtcccacaatcaatcacctaaacaagaaaaagaaaaagaaaaaaaaagtcaaattggactaatgcaaaagtggcacaatctttgtcttgaactctttaattagcaacaaattatactcttctgcacaaatcacaagaataccttacatgaaaagaatgttaatttgacggtttacattaaattatatgcagttttctgcaggtaattaattttcaagaatttaaggtggttggtaattttcaatagctagcttgactagcaaaggaaagaataaagttaaaatgcttcttgttttggcctttttggattgttatactttttgctaaacggaaatggttatatgaatggtaaaggagataaattgttacatagtctaaaattgttatagtcttaatcaatcttcaaacaatatataatataattttattaactattttttataaattaaaattttcaattatatgtcacataaaaaggaattatgaccataaatatataatagtcttaaatcatgtaataatttgtcagcaaaataagagattaaataaagttagaaggaacacaatcagtggattaattaaaacccgtcatataaactaaattaaatatcaaatctcaaagtacgcataaaaaatctcaatagatttttgttaataattaactagtattcttaaaatattgattaaaaaattaaaagaaaaaatatatttattatataaatcagaaaaatataaaaaaattataaataatacaattttttttctcctaataacaatatttttatttgatgtccttagaatattgggctaacatttaacatataattatacttgggaaaaatttactagaaatagtaataatagattctctaacactttctcctaacatagtctatgattaatttaaatttattgaaaactatgaagttatgagagaattattatttttgtaatttttaagaaatttcaactagagaaaatatgtttaaaagagcctgctgttaaactttctcaaaatttattttcaacctctaaccgcagacttctgaaataagcattcatgcactttattaactagcgggtgcaacaactcctatgggtttggaaccaagttaagtttccctttggggtctgacctcaactaaattaacctaatctgcctaacctcaaaggactttatctctcccccacctctaatccaccctataaaagcaccctctcccactcttacttgcattgcaaccttaaccttcagcattcacactaaggtgttccttgctcgccaaaagatcatggagcctgccaaaaccattcacaacaatgtcaaatactcccccatcttcttagccatctttgttctgatcttagcttcagcattgtcttcagcaaatgccaaaattcacgagcacgagtttgttgtacattctctcactctctttctcttaatttctctggacttattttattcttgttttttttaactcttttccgttagataataattacctagctgttgattgtaatggaataggttgaagcaactccagtgaagaggctgtgcaaaacccacaacagcatcaccgtgaatggacaatacccgggcccaacgttggaaatcaacaatggagacactttggtcgtcaaagtcactaacaaagctcgttacaatgtgaccattcattggtataatatcaagctagcatcttaacttcattttagattatgaagaccctttgacttaattttacacatctgcttaccacgatttatagacatatagatattagtcttacgaaaaaattttttctcactttataattatactactcccttcactcctttttatattaaaatggaaagagtctctctctaccatgtgaaaaataatatataaataaaaacatatatactctaccatgtgaaaaataatatataaataaaaacatatatacactttttacttcttgacattctaattttttttacctttctttctttcccatgatatttatcatttattctctcctctattctcatttatctatctcccaaggtgtgtacattccattagaatgtgaaaatgaaaaacattcacaagcataatgtaaaaaaaataatatt

SEQ ID NO: 9 - DNA, 2114 bp, Glycine max
tctttgttaggatatcatttcaaaatattcgttggcccccaacaatcggagttttgcagtttttcttagtttggaagctggttgatgcttccctgcattaattttgggagggttttttgttttgcttcattgctttgttttaattgtacgttctttttctagagagagataaattgggtttggaacctaggagaggtggtgatgatgcggtttcaacgcaactataccatctgaatagtcattgcccaactagttaaattgatgttattttccccccatagctcaagttacaaacagattgcaagttttgaatatcaatatatcagctggcttttttttgggttgcaactcttggtctcttgaatataaatatgttgacatgtattaagtttcaaagtcccctaaccatttgccatcataaactaatacaaaattctcacattaattcacagtatagtgtatacagagggatgcttataatagcatcaagcattaaaatccctcaacgaatgttgtcaacatatggattatgggataaaaatataactacgcttttctgatagagggtggcttttggttttcatcaggctgaatgtgcaagctgatagaggagtattaagatagagtagactattgtaattgagaagcagaaagctactgtcgctgatcatgtgagtgggtggcaaatgtactagtccagtagtcctcttgagttttagcccaataatctcatcaaatgaatctctgggcctgcttcattaatgctaaaagttgcctcttcggcctatgctttttttttctttgttaataaagtgatgaaaataaaggatatattcataaactttatttatgtttactttgtataaatttcttaacaaatacttattgagaaaaaaaaatcaaacttctctcataacataaaattaacttgtgcatttctaactttttagaagttttcttatttaacttccctaaaaaacattacatttgctacagtaacttggtgacagatttcatgttagctctcgcatgatttcaattgtattacaacatttgtactctcagttatccttaataaaatgatatgattttccttgcctaaaaaaaacttggtggcagattttgttgcatagattaattcatctatgtgcctttaagcatttgctagtcatgatggtctataaatgcagtctttacatgtatatttctagattttaacattgttgggttctgtactatcttttggggtctttatttggattcaccttgtgcttaaacctaaaggctattgatcattttattctttttctttttgaatttttttcattaaagttttttctttttataagttgattttaatttttagaagaaacttaattcattttctctcttctttttcctcctgatgaaaaattattcaaattgacttttaaaaagattgtatcatatatatgtgacattttctttttcattattttttatgttcatgtcacataaaatgcaagtctcataaaatgtggttgtaggacaaaaagtgatactatactccacatggtatatataccaaaataaaagtaatacacggaatcatgtgaagcctactcaagtagatgtgcaaaatcttgtgaattcaaattagttgtcttgtttattcattaccttttcaatttttttaatcaccataattaaggcctttcgaatccctttaagtgataaaagaaacgtgcaattatgcaacaaataaattttcgttatgttactatttagtcaaggaggaaaaaaaagtgataagggaagaaacaagggatatttcctgttataacaaacttaaaatggcgactattttgacgacattgcaaatactcatagtacgatataaattttgaatttaatatacaatgaataggcatattcattttctaccccaaaaaagcatactcatttatgtacatttaattttctctccatagaggaattaatgtacaaccatgcataagggatgagcgaaagggacagattattgcaatccagaagcatccaaggaaagttggataaacaaatcaattaatatatataaaaaaaaaacaaaaatgctcctagtagaagattaaagg

SEQ ID NO: 10 - DNA, 1760 bp, Glycine max
tttcactcattatatatatgtttgtaccaagcaattagtgttgacaagaaaaacaaaaacctttctctcttgggttgttggtgtttgcaacaaacgcaaacacgagcactacctaagggagaacaaaagtagataggagaaaagcatatatgtgcatttttatatgctctaatcaaattggcagataaagcaattatatttaacaaaatatgagctgcgaatgtggtgttagttattacaacggttcaaaaatcaattatagcaatgataactttagccaaatgtattccgagttggagtttcgagaaattttggctaggaaagaaaattgatatgctatcactgagctaaggccagtgtgtaacgaaaaaccaagaagagtgaatttcaattttgtctctttaaggtataattttaaccaaccgtgtcacaaatatcgcacaacaaaagtttgtgtacaaatggtttaatgaagacaggtgtagtcaacgagaataactggttacaaccaacaatgacatacggaatagtggcagggatgatcggaaaaaaaatacagcaaataggcaatagtaaattttaatttgtgcgccgtttcacgataatattttcttgtagaaaaggattctccttgcaatagaggagtacaaaaagatttgtacaaactactactagcatttaataataaaaaataattttgataacaaaatataaattaaaatatgaaaattcatgcatttaaaattttaagtgttgaaatttccttaaaaaaatagtttgattgattgatataatatttctttattttgatatatatatatatatatatatatatatatatatatatatatatatatatatatatatatatatataacgataattgataataaaattttacttttacttttaaatacatatataaaaattaaggaaatagtatgtgattaattttactacttttctataatatagtttaaaataagaaataaaatcaagacacgtacttgtttgctattattatttaaattagaaaatgaaataaatattttattgaacctaaagtgtctttaatcatttagcatggagatcatttggaaaagttgttataatttaactagatgtccgtttgaaaaagtttatgtgtatcttaaagttatattattgtataaagtgtatgtttcgaagatcttattaaaataatttataatatatatctatgataaagactaacaataactaaaaaaaactttgcatcacattgttaatcttttacattaatttaaaatataattcacatattttattttttatttattatgaattttaattataatacatattcaaaattatttatttattataaattttagttaaataaaataaacatttattttgcaaattctacaagctaataaactagtattactaaatgcatagagtatgagcaaattcgtgtaggtggattttgtatattatatatgcttttcgttaaataattattactcgactttagaaggttgaaaaaggtcataaaaatttagctattatgaatctaaactttctttctatttttcttccgatccttttgttttttcatcacattagttatcgtatatgttattttttcttgatttttttcctatctctttattccttctacctaatacactaaaaatgatatatatacatttttccacaaagactcctgcccttttctatcagctaaaattatttatgtacaaataaaaaaggtacaa

SEQ ID NO: 11 - DNA, 246 bp, Glycine max
aagtcgccatattatctctatatatatccaactaatcaaattagtatttctgtgtttgtgtttgcatgcatgatgacgcaacacaaacacgtacccagggacataaaatgctgagtggagcacgcaatgatatgaaaaaacaatgtcttaattttttgttgttgagttatgaagaacaatgtctgaatgaaacaaatgaaaaaagaggaggggaggggtcttagaagctttagattaggaatcgga

SEQ ID NO: 12 - protein, 271 aa, Glycine max
MSTSSSSQSLKIGIVGFGNFGQFLAKTMIKQGHTLTATSRSDYSELCLQMGIHFFRDVSAFLTADIDVIVLCTSILSLSEVVGSMPLTSLKRPTLFVDVLSVKEHPRELLLRELPEDSDILCTHPMFGPQTAKNGWTDHTFMYDKVRIRDQATCSNFIQIFATEGCKMVQMSCEEHDRAAAKSQFITHTIGRTLGEMDIQSTPIDTKGFETLVKLKETMMRNSFDLYSGLFVYNRFARQELENLEHALHKVKETLMIQRTNGEQGHKRTES

SEQ ID NO: 13 - protein, 673 aa, Glycine max
MKPHTPASSFVTRLPHVPYFRGATAARAAPPDPPHDAPGGLEFRRVSTAKRRRVSLSVCHASRVTAASNPGGSDGDGDTRARSSRRGVLMAPFLVAGASILLSAATARAEEKAAESPLASAPKPEEPPKKKEEEEVITSRIYDATVIGEPLAIGKEKGKVWEKLMNARVVYLGEAEQVPVRDDRELELEIVKNLHRRCLEKEKLLSLALEVFPANLQEPLNQYMDKKIDGDTLKSYTLHWPPQRWQEYEPILSYCRENGIHLVACGTPLKILRTVQAEGIRGLTKDERKLYAPPAGSGFISGFTSISRRSSVDSTQNLSIPFGPSSYLSAQARVVDEYSMSQIILQNVLDGGVTGMLIVVTGASHVTYGSRGTGVPARISGKIQKKNHAVILLDPERQFIRREGEVPVADFLWYSAARPCSRNCFDRAEIARVMNAAGRRRDALPQDLQKGIDLGLVSPEVLQNFFDLEQYPLISELTHRFQGFRERLLADPKFLHRLAIEEAISITTTLLAQYEKRKENFFQEIDYVITDTVRGSVVDFFTVWLPAPTLSFLSYADEMKAPDNIGSLMGLLGSIPDNAFQKNPAGINWNLNHRIASVVFGGLKLASVGFISSIGAVASSNSLYAIRKVLNPAVVTEQRIMRSPILKTAFIYACFLGISANLRYQAVFEVDGG

SEQ ID NO: 14 - protein, 100 aa, Glycine max
MEPAKTIHNNVKYSPIFLAIFVLILASALSSANAKIHEHEFVVEATPVKRLCKTHNSITVNGQYPGPTLEINNGDTLVVKVTNKARYNVTIHWYNIKLAS

SEQ ID NO: 15 - protein, 278 aa, Glycine max
MGTMSHVRACLEKQAVLPIHNARWNSKRRLFIQHLAYGQKHINSHMKGKSTLVSSAKTAEAINTSNSDASSDNTPQGSLEKKPLQTATFPNGFEALVLEVCDETEIAELKVKVGDFEMHIKRNIGATKVPLSNISPTTPPPIPSKPMDESAPNSLPPSPPKSSPEKNNPFANVSKEKSPKLAALEASGTNTYVLVTSPTVGLFRRGRTVKGKKQPPICKEGDVIKEGQVIGYLDQFGTGLPIRSDVAGEVLKLLVEDGEPVGYGDRLIAVLPSFHDIK

SEQ ID NO: 16 - protein, 298 aa, Glycine max
MANNFLDVFCWIQNLPPISEWETSSMSLNICSSSSSCQPRLNLTAILLYGSNKNSTTFIRFPNLDSTASDNLSDVFNLSDSRQASHMIMKLLGSNLEELWMRSLNLAVQLYCHLMVMDVENSKSSPASERLQFSLRYHHVEGVLQFNHKVLIKDEWAEIMVDIDNVRCDVIELVNEFLMKQRGAGAAEKHFPSRISLQLTPTIQDQVLSLSVGKSSENPRKEIGVDKSVEASFEASNPLALKVSAGESPQPLVYGYSANLNWFLHDCVDGKEVLSSKPSKFAMLNPKSWFKNRYSSAY

SEQ ID NO: 17 - protein, 502 aa, Glycine max
MAFFSGHGVRQMRTGWADGPEFVTQCPIRPGGSYTYRFTVQGQEGTLWWHAHSSWLRATVYGALIIRPREGEPYPFPKPKHETPILLGEWWDANPIDVVRQATRTGGAPNVSDAYTINGQPGDLYKCSSKDTTIVPIHAGETNLLRVINAALNQPLFFTVANHKLTVVGADASYLKPFTTKVLILGPGQTTDVLITGDQPPSRYYMAARAYQSAQNAAFDNTTTTAILEYKSPNHHNKHSHHRAKGVKNKTKPIMPPLPAYNDTNAVTSFSKSFRSPRKVEVPTEIDQSLFFTVGLGIKKCPKNFGPKRCQGPINGTRFTASMNNVSFVLPNNVSILQAHHLGIPGVFTTDFPGKPPVKFDYTGNVSRSLWQPVPGTKAHKLKFGSRVQIVLQDTSIVTPENHPIHLHGYDFYIVAEGFGNFDPKKDTAKFNLVDPPLRNTVAVPVNGWAVIRFVADNPGAWLLHCHLDVHIGWGLATVLLVENGVGKLQSIEPPPVDLPLC

SEQ ID NO: 18 - protein, 763 aa, Glycine max
MSESSITTVFNDARTGQMIVCLKNDRTVGSLGVWMGDNPFDFVVPVTLFQIILVSLLSKALHYVLRPINTPKFICCVIAGILLGPTFLGRHEEILGALFPVRQSLFLNTLSKIGTTYCVFLTCLKMDVVTTLKSAKRCWRFGVFPFLASFLVTVTLFSLYSPNGNANQNQMSIYHFPNIFTLSSFAVVSETLMELNLVATELGQIALSSAMISEILQWTTMELLFNSKFSMRFLIVLLIGATGFAVLLLLIIRPLVNIVLERTPPGKPIKEAYVVLLLLGPLVMAAISDTFGIYFVMGPFLYGLVLPNGPPLATTIIERSELIVYEFFMPFFFLLIGTRTDLTLIHEHWEVVLVVLAILFVGCLVKVIDTEVFSVAVMSVVVMTSICIPLIKSLYRHRRVCKTQTIQEGSVKTIQNITENTPFNIVSCVHTDEHVHNMIALIEACNPTTQSPLYVYVVHLIELVGKSTPILLPMNKNKRKSLSVNYPNTNHILRAFENYSNNSSGPVTVLSYVNVAPYRSMHEAVCNLAEDNSVHLLIIPFHQNDQTLGSHLASTIRNLNTNFLANAKGTLGILVDRYSVLSGSSSKLSFDVGIFFIGGKDDREALALGIRMLERPNTRVTLFRFVLPTNEDSRFNGLVENEDENLESTLDESLIDEFIAKNDISSDSVNVVYHEAVVEDCIQVLKAIRGMEKDYDLVMVGKRHSMGNFVEEEMSNFMDNADQLGILGDMLASNEFCNGKVPVLVMQCGDEKRVKQLEKVCHI

SEQ ID NO: 19 - protein, 871 aa, Glycine max
MATSRGNGIVSSYWDSHGQWQVCVEDDRNVGSLGIFIGDRPFEFVLPASKNTQIHLQRPVSPLENSRKRHKLDRCTSPTRQCDNVVYSSKLKVVEAVPNYSSKSPMLDSTSPIVVEECLRYGGGIILGPTFLGRNKTYWQVLFPPRQTEYLVMASLTGAVYFVFLVALKMDVLMTIRAAKSTWRLGVIPFLASFVVILALLCLYYHPQQISSASLTIARVSVSCLMSLSNFPVVSDAMLELNLTATELGQIALSSSMINDIILWLFIVMHSFTSNVDVKKSIALLGNWCLLVFFNFFVLRPTMKLIAMRTPVGKPVKELYVVLILLGVLVMAGVGDLMGVTFLMGPLIFGLVVPSGPPLGTTLAEKSEVLTTEFLLPFFFVYIGINTDLSALEDWRLFLTLQGVFFAGDLAKLLACVLVSLAYNIRPKHGTLLGLMLNIKGITQLISLARFKKQKMLDEDTFSQLVFCVVLITAIVTPLVNILYKHRPRVHAESLFEGELRTIQSTPRNREFHIVCCVHNEANVRGITALLEECNPVQESPICVYAVHLIELVGKSAPILLPIKHRHGRRKFLSVNYPNTNHIMQAFENYSNNSSGPVKVLPYINVAPYKSMHDAIFNLAQDNMVPFIIIPFHENGNIDLVGHVAASIRKMNTRFQAHAPCTLGILVDRHSRLGASNNNNMYFNVGVFFIGGAHDREALALGIRMSERADTRVSLFRFVIVNKKPCGCKIILTREEREEEEEDTMLDEGLIDEFKSMKYGIGNVCWYEITVDDGVEVLEAVHSLEGNYDLVMVGRRHNDGSLNGKEMTTFMENADALGILGDMLSSVEFCMGMVPVLVTQCGGVKISSSSNNKLDRVGSVNVSQKRLSVHK

SEQ ID NO: 20 - protein, 1245 aa, Glycine max
MEASVLLGNSYGVVARVAVAGLRGGVMAFEAYLSRSWRAVELIKFESGTTTLYFVDNHHMTIKKGSFSDVRVRSRKATLSDCSFLRTGIDICVLSASQGNDNSDESSANHVWLDAKINSIQRKPHNPECSCQYYVNFYVNQGSLGTELRTLRKEVKVVGINEIAILQKLERNTCQHKYYRWESSEDCSKVPHTKLLGKFISDLSWLVVASAIRKVSFCARSVENNIVYQILGSDATTSSLYMDSEISVVNFKVNEDGMQMPVIHLVDLFETDTNTSGDKHDSHYDEVPSSYGFEGLRRSKRRNIQPERYSDCGNVSEIKVGNVRTWPYKLNKRKDDDGGGEESLPLAQENSDNSQKVNELSSCREIIVYHGRNETLELKSGEANQTQLASVPLLQEGDSLALEHHHLNDNVTRRSDAYYSTPKLKRKRLVDLEADVDFDPGREGINSNKGVSEKRHGSSWYSRSRSHAAEHSYKDRSLNATAYKEMIDSYLKDVNRTPTTEEPPVMDQRKEIGNFGQKKEAEIPEREDEEQISEIDMLWREMEMALASSYLEETEGSNSANFAKTTEESNRTCPHDYRLSEEIGIYCYKCGFVKTEIKYITPPFIEMQRSVRHQEEKQCNGKDTKEKASKDDDFHLLSTHAPTDEHNSMEHDNVWKLIPQFREKLHDHQKKAFEFLWQNIGGSMEPKLMDAESKRRGGCVISHAPGAGKTFLIIAFLVSYLKLFPGKKPLILAPKGTLYTWCKEFNKWEISMPVYLIHGRGGTQKDTEQNSIVLPGFPNPNKYVKHVLDCLQKIKLWQEKPSVLVMSYTAFLALMREGSEFAHRKYMAKALREGPGILILDEGHNPRSTKSRLRKGLMKLKTDLRILLSGTLFQNNFCEYFNTLCLARPKFISEVLDTLDPITRRKSKTVEKAGHLLESRARKLFLDKIAKKIDSGIGNERMQGLNMLRETTNGFVDVYESENFDSAPGLQIYTLLMNTTDKQREILPKLHTRVDECNGYPLELELLVTLGSIHPWLVKTTSCANKFFTADQLKQLDKYKYDMKAGSKVKFVLSLVFRVMQREKVLIFCHNLAPVKLLIELFEMFFKWKKDREILLLSGELDLFERGKVIDKFEEHGGASKVLLASITACAEGISLTAASRVIFLDSEWNPSKTKQAIARAFRPGQEKMVYVYQLLVTGTLEEDKYKRTTWKEWVSSMIFSEAFEENLSHSRAVNIEDDILREMVEEDKSKTIHMILKNEKASTN

SEQ ID NO: 21 - protein, 28 aa, Artificial Sequence
  SYNTHESIZED
CTLSRLKTLDISNNALNGNLPATLSNLS

SEQ ID NO: 22 - protein, 172 aa, Glycine max
MPNPKVFFDMTIGGQSAGRIVMELYADVTPRTAENFRALCTGEKGVGRSGKPLHYKGSSFHRVIPSFMCQGGDFTAGNGTGGESIYGAKFADENFVKKHTGPGILSMANAGPGTNGSQFFICTEKTEWLDGKHVVFGQVIEGLNVVKDIEKVGSGSGRTSKPVVIANCGQPS

SEQ ID NO: 23 - protein, 763 aa, Glycine max
MASHIVGYPRMGPKRELKFALESFWDGKSSAEDLQKVSSDLRASIWKQMADAGIKYIPSNTFSHYDQVLDATATLGAVPPRYGWTGGEIGFDTYFSMARGNATVPAMEMTKWFDTNYHFIVPELGPDVNFTYASHKAVDEYKEAKALGVDTVPVLVGPVTYLLLSKPAKGVEKSFSLLSLLPKVLAVYKEVIADLKAAGASWIQFDEPTLVLDLESHKLQAFTDAYAELAPALSGLNVLVETYFADIPAEAYKTLTSLNGVTAYGFDLVRGTNTLDLIKGGFPSGKYLFAGVVDGRNIWANDLAASLTTLQGLEGIVGKDKLVVSTSSSLLHTAVDLVNETKLDDEIKSWLAFAAQKIVEVNALAKALSGHKDEAFFSGNAAALASRKSSPRVTNEAVQKAAAALKGSDHRRATNVSARLDSQQKKLNLPILPTTTIGSFPQTVELRRVRREFKANKISEEEYVKSIKEEIRKVVELQEELDIDVLVHGEPERNDMVEYFGEQLSGFAFTVNGWVQSYGSRCVKPPIIYGDVSRPKPMTVFWSSLAQSFTKRPMKGMLTGPVTILNWSFVRNDQPRSETTYQIALSIKDEVEDLEKAGITVIQIDEAALREGLPLRKSEQAHYLDWAVHAFRITNVGVQDTTQIHTHMCYSNFNDIIHSIIDMDADVITIENSRSDEKLLSVFREGVKYGAGIGPGVYDIHSPRIPPTEEIADRINKMLAVLEKNILWVNPDCGLKTRKYTEVKPALTNMVAAAKLIRNELAK

SEQ ID NO: 24 - protein, 12 aa, Artificial Sequence
  SYNTHESIZED
RLAPGGPDPQHN

SEQ ID NO: 25 - protein, 28 aa, Artificial Sequence
  SYNTHESIZED
DLPLAPADRLAPGGPDPQHNVRAPPRKP

SEQ ID NO: 26 - protein, 12 aa, Artificial Sequence
  SYNTHESIZED
RLAPEGPDPHHN

SEQ ID NO: 27 - protein, 14 aa, Artificial Sequence
  SYNTHESIZED
AHEVPSGPNPISNR

SEQ ID NO: 28 - protein, 15 aa, Artificial Sequence
  SYNTHESIZED
SKRRVPNGPDPIHNR

SEQ ID NO: 29 - protein, 31 aa, Artificial Sequence
  SYNTHESIZED
RAELDFNYMSKRRVPNGPDPIHNRRAGNSGR

SEQ ID NO: 30 - protein, 12 aa, Artificial Sequence
  SYNTHESIZED
RTVPSGPDPLHH

SEQ ID NO: 31 - protein, 30 aa, Artificial Sequence
  SYNTHESIZED
KGLGLHEELRTVPSGPDPLHHHVNPPRQPR

SEQ ID NO: 32 - protein, 13 aa, Artificial Sequence
  SYNTHESIZED
KRLSPSGPDPHHH

SEQ ID NO: 33 - DNA, 78823 bp, Glycine max
cggctggttgcgccaagatccgcttgcggagcggtcgaacatccatgctgggacttcaagaagtcgagcagaagaagaaccagaaaggctgcaccggaaaatatgcgtccctttggagagcgcctcatggacgtgaacaaatcgcccggaccagggatgccacggatacaaaagctcgcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcaaattacaacggtatatatcctgccagtcagcatcatcacaccaaaagttaggcccgaatagtttgaaattagaaagctcgcaattgaggtctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgtttggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgatatcaagcttgttggttgcagaaaatcatttcgatgagtttttttaacggacaactttataatataattattttgaagaaaaagtgagtttttctttctaatttatgaaaaaaatatcaaattatatattttttaaatttgttttgctaattttcgctaaaacttgcaatttttaaactatattttttcttaaaagtcttctgtctttttttttatttttatttggtggggaagacactttttgtctttaaaagagaataaaaagtagaaaggccatcaaatagacgctgtaccaaaaggcaaacctattgttgacttgtttcataaataattgacatctttaataatttcatatcatatgtctttttcctaaaaacgcttctctttttcatgactacatatcttgttttaattatgtcaaatcttttgcaaagtaaatttattaaaacaagacatgtgacatgttcattgtagtcaaaattaagaagaaatcaaacctttgctagagcacttgtaaagatcaccaggttgaccattgatagtgtatgcatcagacacgtttggagctcccccagttcgcgtggcctgcctcacaacatcaatagggtttgcgtcccaccattcccctaaatattcaccatcatcactttcaccgtttaatctttgtccaaaagcaaattaaacaacaaaaaacaataatttctctctctctctctctctctctctctctctctctctctatatatatatatatatatatatatatatatatatatatatatatagtctctttgcttcaaaagtagtattaattaattccaactatcacaatcactttttagtttttaattaatcttaattcttttttagattgttgcctccatgtttttcatcaatgttataattacaaatagtgcatgcatgggtaaattaattaacaaggaaaaattaccaagaagaatgggtgtttcgtgcttaggcttggggaaagggtagggttctccttccctaggacgaatgattaaagcaccgtaaacagtggcccttaaccatgagctatgagcgtgccaccaaagtgtgccttcttgtccttgaacggtaaaacggtaggtgtaacttcctccaggacgaatcgggcactgagtcacaaattctggtccatctgcccatcctgttctcatttgcctaacaccgtgcctaagaacatatattataattaagaaagaatacgagcaataaaaatcactttcaattatattaattcactcctaaaatgaagtcttaatttggactctttcttgcttttaaaactttcttgatggtatcacatacgtacgtacgtacgtatcatgtggtatgtattaggattatgagaaattattagttttacatatgtggttgtcaatgcttttcattttcacattctaatggaacgcattcaccctttgggagatagaaaaatgagaatagaagagagaatacatgataaatatcatgagaaggaaagtaaggtaaaaaattagaatgccaaaaagtaaaaagtgtatatatatttttatattattcttcacgtggcgtagagagagactctttccattttaatataaaaaggagtgtaaggagtagtatatgtatatataacgtacgtgagaaaaatgtttttcgtaagagtgtaatgtctatgtctataaatcgtggtaaggactaaggagacgtgcaaaattaagtaaaagtgtcttcataatgtaaaatgaaattaagatgctagcttgatattataccaatgaatggtcacattgtaacgagctttgttagtgactttgacaaccaaagtgtctccattgttgatttccaacgttgggcccgggtattgcccattcacggtgatgctgttgtgggttttgcacagcctcttcactggagttgcttcaacctattccattacaatcagctaggtaattattttctaacgaaaaagagtaaaaaattaaacaagaaaaaaataagccccgagaaaataattgagaaagagaaagagggtgagagaacgtacaacaaactcgtgctcgtgaattttggcatttgctgaagctaagatcagaacaaagatcatggctaataagaagatggaggagtgtttggcattcatgttaatgaacttgacagactccatttttttatgagcaattaacaccttagtgtgaatgctgaaggttaaggttgcaatgcatgtaagagtgggagagggtgcttttatagggtggattagaggtgagggagagagataaagtcctttgaggttggcagattaggttaatttaaggtcaaactcaaaagggaaacttaacttggttcaagcatataagagttgttgcacccgctagttaattaagtgcatgaatgcttacttcagaagtctttagttaggggttgaaattgaatttgtcccaagaataattatcttgagactttgtttagtttatatgacgggttttaattaatatgctggttttacactttctagctctcattcaagaggcacgtaacaaggttaattaatcccttattttaccaagaccattatatatattaatcccttatatgttcctgattcatttttatttgatatataactgatatttttaatttatactaaaaactagttaataaaactagattatatatatcatgtggagattgactgtgaataataattttagattatgtgataatttctctcctttaccacttatatatatccattcctgttttaacaaaagtataacgtacaatccaaaagcaaaaagactaattaaaacagaaagcattttaactttattctttccttagcaagtcaagctcgccattgcaaattaccaacagccttaaattcttgaaaattaattacgtgcagaaaactgcatataataatttagtgtaaaccgtcaacttaacattcttttcatgtaaggaattcttgtgatttgtggaaaagggtatattttgatgctaaagagttcaagccaaagatcgtgccacttttgcattagtccaatttgacttctttttttttctttttcttatttaggtgattgattgtgggacaaagcgcccaataatatttattctctttggtcattaaaaaaaagctttaaatcctaacctctaatgataatttttttaatgaaaaagtaatccgcgttattcaattattctttatacccggataattaattaggctaatagttttgattgattatacctagataagtaccgtagacttccatgatgcgtgcagcaaataaaataaagtactataattaactaaagtgatttgattgcatctaacaagaatttagttagagatacttatgaaaaatatttctcaagtattaaattaggtggtctgtcatccaacataacttaactaattttattttacgcatggatgagatcagcaaagaattacacgcacacgaatgtcatactcaaagatacgtacttggatgtaccaggcacacatcatttttatttttgactaacattaggtcaccatactgccacgtgttcagctataataccattaccatggcttaaaatttttcaaatgttagacaatatataggagaatatggtttttgctatattttcaatgatctgttaattgtttgattcccctcaattgttttttttaacacagctcgaaagctaagtaatgatcaattacttaattgaaaggacctctttatgttacattaagaattaatttttcatttggtaaaaaaatgttttttcataccatacatatataggatacgatcaaatcatattagatgtgaaactttcacactttcattacttaagaatataaattaattcatataataatcattgagttgaaagtttttacaaaataaaaaatttatgaaaattcatacaaattaaaaagtaataattatttattatattatttttattttgatagataatcttatttagaatataaataaatgcttgttgatggctcataatataacaaatgttcaatttatataaaaaaaataacatgcataattaaaaaagataatatcaaattacttgagactttcactggggtaggttgatgaattgtttttattcgtaggaatcagaaaaatgatttataataattcacttaatatgttgaatcaattctactagatccttctttataagttgcgatttaacataggtaaactaactgcaatttaattttatttgcttggtctattttgggaggatgagttgtattttattttgatatatggtgggtggaatttattaagagagggactcttatagttactcttggaataacttgatcaaaataaatagttaacatatatatttaaatgattgtattaacgtgatgattattatgatttggtagaaaaaatatcagaaaatgataatgatttctcataatataatgtatttatatattttaaagttaggtagataaaattgaatttaaattgttagatataattaaaatacattaacatgacttttaacaaattgatatataaacacttaaaaaattttcaattaatgactatttacatatattgttattatgaaaataacaccaattactaattagaaatcatcattgacattgcttttaaaataattttataaaaatcgataaatttattataatataaaaattgtttaataacgtaagtgtacatcttattttctaaaatataaataaaaattactagaccaagcaatatggaccttgtgctttctgaacatatataacacgaaaattgcataattatttttccaccttcaagctaaatccgcaacgtactgtggaccctcatgagtgtgaggatcttccacaggtcactcacttgctttgacatctgaaagatccttctcgagtaaagcgtggaaacaagaacaagcttggactggtctacgatttagtgttcttattccaagtaatataaaaatcttaaatctcaaggaatgatgcaatgcttcctctaattgtcagatatccctcccacgcacctaatctgaatgttgtgtctaaattggcataaaaggtttgattgatgaatgatgtctacatgtgagtttgataacaacagctaccccttagccaagccactaactaggacattagttttggttggttgtcagacaaaccgttagaccctgagaacgaaagcgtgtcaaacaaaagataatagacttcaattaatataaaaagagatgatcagaaaccaaattgagatttgataggtgaactataaatcatgacagtgcattagacaagttggtagagtttgttactgactcatctgattcttaagaaaggcaaaaataggaactacaccagatgtcgctagctagcgatcgataacgtgcaaattataaataaatggctttatttgaaagttcatcttcatatggttagttataattgggtcaaattcttaatttattagaaacgtgcacttcattttgagtgtataaagttggaagaagaaaaggaatatagaaattaaaaaaatagagaaaaatcaatagatgtaataattaagttatgtttatgtgatgggaaaagaagacaaaataaaaatgagaagaaattcttatgagattgaaaagatatgaagtatatttttttaagatgatatttgtatacaataatttttgtcgaagagaagattaatctttagtaactaactaaaaatacacataagataattatttttcttgtaacataatttattttataaaaaatattaatcagaatttaaatatggatcatttgattaaaaaacataaattgttaactagtgtatatttatatacaaaaatccaattaactatctaacttttgattccaccacacatcaatatatattctctctgaccgaaaattatgatggttagtaaacgaaattaaagacaaatacaccatgttcttttgtttcaaaatggggccttcttaagggtcacgcccaatgatacgttgttgtttaagttgttaaaatgcatgttaccctcctcatgaagctataataggtttcagagatagtctttagaagttcgatagaacatgtggatctgggagtgagagtgaggtcgtatgtcgctataatgctgtataaacttttggtgagcatgcattgccattttgtgggtttcggtgatagtcttctcaagttcattagtctttacaagttcaatatagatagatagaatttgtggaccttggagtgagagaggtcgaaagtcaccataatgctgtataaacttctgatgggcatccaatgtcccctaggggcaacagggtacctaattaattggtaccacaacggggagaaaatcgatcaacacgtttgtggaatatacatatatacctagaattgaagggcaagctcaattaatcaagttaaacatgaattcaactaatagaaattaaattgaaattttgttactcgggaccattagttataataaaatgcgttgcacaacttcagttattttggattttttgttgagtaactcaatcaattttaaatgttgctttctaaatcaactattaaaaaaactaatgcagaaatacatctacatcgtggctagaatcgatctagaaaactttttttaatgtaattaatttctaactttttgcatcaatattatgaatagagtcaatctaaaaaaaactatatatattggttctagtctttttggcatagctacaactgatttaaaaagtgactttcattgattacgattatacaaatgtggaaagactatttaaaatttatatatatggaagagaattcagatattttaagagtaagtataaaaaattacattaaaaaaaacttcattgatatattaaaaaaaatcataatattataaaaaccgaataattaaagacgtgcacattagtaatgtactatggtagaacatccaatgcacataattgtttgttggtcaaatgatattggcagtactctttgatctacccatgagatggtgtctcttcagcaccaacttagaagtgttagtctgaagattgtttgtatatgtggttttggttgatactaggttgttggcagtggttgaaagacatttcctcttggattatcacacaatcatttgtgggattttttatgttttgcttaaatattcttcatttaaagtttcattactaattatcagtttaagaaataaatagaacaagtagtattgatattaaattttgcaaattatatccaaacctagtcgctaacaatgtctgaaatattgttacacttgattgtgtgatactgtgatctatttatgttttcttttttgactgataagtttgatttaacgataggattatttccatcctctttttaggctgacattatatttggataaaagttagaaaagtattgttgtgatttgtgaatttcaatatgcatatttctaaatttcgatgtacaaaatgagaaagaaagaaattgttgttttagaagtaaaactctctcaactgaagtacaaacatgaactgaatctcttgtgaggtttccacatgacatgggggaaacaatgggtgaataaagattgggttttgtaagacttaatttgagaattgagacagaagaagccttatttgtttgtgggagaatacgagtaactgacatgagaaatgtggcaatctgcaactattaaaccaaagaagattataaaaagatatatatcaaaacctcaaacatttggataacattcttaagcttgtaatattaatgtgcataagagactttcacaatgacagatctgcttaagcttgtaacatttggataaacattcttttttgtttctttttgtcattggttatgaattagtgtatgctttctttgtttatgggttttgtttccaagtaaatttgaatgttaaactttttattgagtaaattttgaagttggaattatgtcgggtggttttttctttcttctttttttgccataataggttctaacttccttaactatccgttatttatcccttttttctgggtctcttatttattccagttagagatatttaattcaagatgtgattcatattgttcacatcagttcaattttatatttctatctccaatagataaattatataaaattagttcaaacgtacagagccagtagtgttggaacttggaagtcattatctttgaagttgttgtcatagttatcagatgtgaatgtggaatgcttgcaggaaaataatattagtaaacttgaaagaatttacaatgaaagatgatgacttcacatgtatagaaggtagcagctgagtgaaagagaatctcgaagaataaataatggatgctcctgagtgagcaactatttcaactattatgtgccttggaggaaacagaattacacaccagctttgggggtagaggcaaaattaaacagaaactacactttgttgtagtatgtagtatgtacaatttcaagcattcaaacaggcaaaattaatataagaatgaaagaatcatagaacatatatatagtactaatagtagtagcctgttcggatacaaaaggtagaaggtgctgcttttaagagcttttcaaaacatctcaactggaaaaaagctctatatttccaggagataagttttcagaagcacttatgacttgtatcaaaacaactacgtttggcacaaaaaaaaaaatcaatgcaaaaattgtgctatatggtatcgtccccaggactggctgtgactgatctctctggtctaatctcttctagctgctggagcacttgatgaacttccgggcgtactgatggagaaggatcaacacagtgcaaagcgagcttcaacgtgttcagcaactcatcgccaactgtcgatgcatctctcatcatgtctgcatcaaaaacctcatttgtccactcctctttgacaatagaggcaacccactgaggcaaatctagtccattcatagacaccccaggtgacttcctcgttaggagttctaacaagataacaccaagactgtatatatcagttttagtgtttgctttcttgagctttgagagctctggtgcccggtatcccaatgctccagcagtagctatcacgttggagttagcagcagttgacatcaaccgagaaagaccaaaatctgcaattttagcatttgtgttctcatcaagcaacacattgctggatgtgaggttcccatgtataatgttctccagggaatgaagacaaaacaagccacgagtcatgtcctgtgctattttcatccttgttggccaatcaatgaaggtttcagttccaccacctgcatcaagatgaacaagaaaatacttcacttattgataagctatatgaacaaaacaaatatgattaaaactaaggtattgttccttaggtgtttttgtttgtactgttctccaattagaattatacatattactaaggtaagtaaaactccaattctaattatggaacagcacacacaacacttaaagaactatgtatgtttaagttttctcctatttgtaatgtaatcaaacttaaagaactatgtatatctaagttttctcctatttgtaatgtaatcaaacttaaagaacagcacaccaaacttaccatgtaggaaagaagcaagacctcctttaggcatgtaatcaaaaaccagaagcttttcccctttgggtcccaagtaataggccctcagagccaagacattggggtgtctaacttttcctagaacactgacttctgattcaaattctctgtgacctttagtgatcttttccctcaatctcttcactgcaacttggcttccatcctccaaaatagccttatacactgttccataggtgctctttcccatgatctcagcagttgcacacaagagatcatcggctgtaaaagccaatggtccatcaaaatggactagtttccctccagcctccccacctgcttcaacatcaccagcagaaactggagggactcctttttctgtccttccagtggctgctctccccgtggcttgtccgttctcagccttcgatgttgatctctttctgatcaagcagaaaagcaggatgcaacaaagtataatcaggactactaggagaactcctgctactatgagaattatgtctttggtacttaggttcctacgatggtgctgttctgacagtacttctggagttggggcaatgactccttgtgatggagcttgtgaaagacatggggttgaagggctatacccacatagttgaatatttcccacaaatgagcttgagttaaatttcttggcaagtagaggtggaacagaacctgaaaggctattgtaagaaacattgaagaaatcaagactacgttgactttcaaaggagactggaatttctccactgagattattcagtgacaaatcaagctgcctaagcatggaaatgtttgcaatgcttgaaggaatatgtccactaaattggttcctactcaaaatcagaacagaaagattacgcaatgtacctaaactttcagggatttgattttcaaggaggttgttctctgcattcagcaatgtaagtgaggataaattagagagggtaacaggcaagctcccattgaaggcattattagaaatgtctagtgtcttaagcctagaaagagttcctatttcatttggaatagctccactaaacttattatgactaagggaaatctcactgagctctcttaagctacccaaagaagcaggaacattaccagtgaaaaaattatgatctaggatcaaattttggagcctaaagaagccactcttgggactcccaccccaagagttaggaaggttgccagaaagattattattttgaagagaaaggaaagtgagagaaaatgagtgagttaggctagttggtaaagtaccagagaaggagttgaaactcaagttaagccaataaagcttggtggaattggcaaggctataagggattgctccggtgagcaagttgttgctgaggtcaagagactgaagcaaaggacagaaacctaaagaagaagggatggaaccagttaacctattgttgaataactgaacccctctaaggttgggaagaagtcccaaagttgaagggattgaaccaccaatttggttatcatgaagactaagcttcctaaggccttgaagttggccaattttgtcagtgattcgacccttcaaacccttccaaggaagctggatcacgataacctgtccctgagcacacttgattccaacccaacctcctgaacaagcaccatagccactgtcgttccagctccgcaagaacccttctgggtccaccaactcttgcttgaaagcttgaagtgctaagaggtttgatgctgtcacaaccactccgtcccaactttcatcttcacacaaagctggtctcacgcatgaagggagcacaacaagactccacatacataagagaaacaaaacacgacatgggttgttgttgtgtgtcttccatctttctttcttcttatcagaaatacggttgaagcattgtgaagtgaggttggttttctccactagtaacataacgaaaaaatcaaaagaataaaggtaatgagaatgagagattttgatttagtggaacaaaaaaacactgtgtgttgtaagttgaagaatggtttttgtagtgaggagagagaggaagagtgaggaggagcacgtgatgacataaagaagtgtaggtgcgcgcgcgggttgagagcgtgagagagagaaaatgaacatgcggggttcagaagtgaggctctcttcgttgttaaagaaacagtgagtaacggctagttttccttgtaaattgtgactgtagtgtgtgtatagttgtttttttctttattcaaacaaatatagatctagtcaagaatggaactcatatctcaagaaatgagttcaattttcattatgaaagaatattattggtaaataatctgatttttatgaacggtatttaagttttgaggtatactttaattttagtaaactctgatatttaatttatctaattttttcttaatacaaaagagaaaaacaattataaattgaccaagataaaaagttttataacattattcaatttgttaactttataataattactttaaaattatattaatagtaaattaagattaatcccatacatgaccattaaatccattaaaaaaattgtttttgttggcaggatttattatagattatggtaagctctttttttttttttcttcattgaaacggggttagatccaagaaaaggattacataagaatagttctagaggattcaatcaccagagatgcctaaaaaaatgaggttgttgctgggaaaccatagtgcaaccctccgtattggcgccctcttcttttattgcaacacccgcgcctctccatcaccaacggctattttcttcttcactacttcacacttagtaggaccatacttcttcttccttgttttcttgcttccacttccgcacacaacaaacactgaaaagcaatactactagctttaatattctcacgggtaaattgtgttttccgtcattaagatcttgtcaaaattattcttagtctctccattctaaaaatggtggttttggttaccatgtgttctgaaacatcctgaatttaattgagataaaatgaagtataagatttggaacaaaattcaatatattttgaaaatacgaatatcaaaaccactttttcagatagaggagactaaaactaattaattttaactaattaaacctcgagaaatctaaaacacattttatttttattttttatttttttacaaataaaataactaattatctaaaaaaattcatttttttaaaaaagaaattcactttgtaacaacaagacaataataaacacatgttaaaagaaattaatcttagcaatccatcctaacacgtttttttttacacactgcgaaaattatgtatgtctcacactctcactgagtaagtaagtagaaaatacatgtaggcttaaataaataaaatcactttcaaacttttattgtaatgttttcattctaaaattagtgtggcttatttgaatttcaattgatgaaagaaaaatgtgctagaaagtgagttaaaaagaatataatactaaccagactatcactcctcgagtctccgtgcattaatgattatgtatgaaaaaaatgttatttttttaattgatgaattgctctgactctgaggccttggctggcatctccaagtttgactgaagtgtcacgtggtggaaggaaggaactaactgggttctgtcaaagacacttgttgcaagagaaatttatcagaaacaggcctggttctggatataagacacaggctacattaacgtccaaaagagaggtaaatgtaattgtttgatgatgatgaccccggttcctaagagttggtagagtagtcgcaattcatatcatagcggctatgaagcacggacacttcagccccttatcgtgtttggtgttcgataacattcatgtcagtgtccgacaccaacacgacacttatactacgttctatattttgaacattacaaatgtacacgtgtgtctgtgtccgtgtcgtgttcgatgtccgtgtcggtgttggtgcttcatagcataggggtcccaaaatctgagaagaattgtcgtgatcatcatgttcatggtacttcgttattggtccacaacatgtttgaaattggaagagctcatagcattagctcatccatctcctttcctttgacacctttgttcgcgtcgttttctgtaagaagaacaacgctcattatattctgtgtgacactaaattttgtggggttttagttttgaattttgacctcccatacaagattttaattcataatccaatgcttgtgctaggagttgtatttatttatatactagtagtttttataatgttgtagtttatttcccttcgttatgttcattattttatcttatatgtatatattttcttttcattgaaaatgaaatttctcaatgcaaatactagtaaaaagcgttattttctcaatgatatacaccttaaagtctcacgttattttctcaattctcataagcgcctttcaagaacaaagccggccattagactcataccaatgtcaaaagcgaacaattgtgatgcaaattccacacagccattatctcttctttcccaatagaatttttccctcaaagatataaaaatctccatatctttagagtccattggccatactattgttaaagcatatcacacaattgaatgtttcttactttcacacaattttatctccccatataactctttttattctttgtgtcgggccaaggatggtagttatgagttatgaccacaagctaggttaatgaaactgatagttatcacttagaagacattaaacgagtggacaaatgacttgggcaaattaagtgacatgggtaaaggctgtcaccatagaaggttgaggtgtgtacactagaaataacccaaatttaaagactgataattagggacgaaacaaaatgacgaatgcattagataaatggttactatcacgatattaatcaagtttcgtctgctggaaaataatattcttcactaggccaatttcaaactcgtgggatgattaggatcacaatgaaggaaaaaccaactaaaggaattgtttacgcctagaggtactcctcaccataatgtaagtaaaaaattattaattcacactagttgataatattagttaatcttatttaaaaaaataaactttattttctaatgtatctttcatgttaattaaaagaaaattttaattttatagaatcttattttttaaatggaaatacttttgttttgaaagtcatgttgatatcaggataatataaaaaaactctaaattgaataaaaaatattttaagaataatttcattaaataaagtgaaatcaatttggtaatgtgtggagtgtggagggactctgccttatctgcatggttgggcttggatgattggtttgattactactttacattattctgttggcttaaaggaaaaacacttttttttataacatttttcacttatttttaaacaaacaagttactataattgaaaaatacatccaatatttaaggtatatataaataatttatatttttaaaaattaaaaaaagataaaatatacataataagtaatgtgctcaaaataaatgaatagctagctaatatcacttatcatcaaaccttatacatagtcagcaaaaaaatcaatcaacccttacatatgcatatgtgtgtacgcatgattataaaaccttacacgtaactcaaattaaacttgcatgttccatatatcttaacaattaatatgaacttattcaccctgtcttattcttaaccagatctcaaattcccttagaaacaaaatttgaaacctgaataatatggacagaaatttctttgaaagagatcatctattacttttaaacgttatcaactaaaaatttggtggcgtaaacattagactaactaactaattacgggctctttggtttatcattgagatttgtatttttcaatgctaaaacatgaattctaatgtaatttttttcaaaacaatttgttctcacttggtagaattctgtttttaatattggacttagatttaattttgctaggtctctttcttttttctcgtttatttcatcggtattctatttttagtaagagatggctttatgtgaaaaaaataaaggcaattaagttataagtggaatattactggtatttaaaaaagtaacaaaaactacattttgttaacgagaattaatgaatagataaggtttaaacttatgactttgttcaaattactcaaattcctcgctatcccaattttagtgatttcataactttctctttacgtgcatgtaattatctctgcctaatgagctacggtctgtcaaactgatttgatatttttcttactaataattgcctgtgcaaagatctttctatctcaatttgttatctatctcggtagggtcgtcactctcaatgccaacaaacatgtgaaaagaagttaattaatgagatccaataaacaacttaccagttaattagctctaataataacaaacatttatcatacacgaatcttcatctgtaacagaatatcttattcatgaagcattacatttatcttattactatgattttcatatacatatggtatattatgtacgtaaatgaatcagtggtccttttacaatcaaaaaggtgacagtgacaagtactaatgaggcaacggatgctcactgtagtgataactctctttggtaaattaaaattgaaattacataaactaccaacaataaaagatatatttttttattttaacattatgatccgtcacgaaaaagtaattctgagtaatttaaaattagattgaaaaaataaaaagaaaattaattaagaataattttcatcaataatcaaactcaaataatatttaaacaatttaattataattttaacacattaatcacatcacatatttactaataattaaacgtataatttgtttactaataatgtaatatggagaaatcaagcctatattcactcaagattatttccttattatagaaattaaatactgtaaggcctttatagatcttaattaaacacttaaatgtgtatcatataagtttttatttgtaagttgtttttataattgaaaagcaaataaggttaaattattttcatataaaattttaattttttttataagttattttgaaaattttattaaaatatattgaaaaaaatttataataaatcataaactataattttttaagttttcttaaatacttaaaggtcacgacatagaataatttcaaataagatataaataaattcctccaagcacatcttaaatctacattttttttaaacaaactttcatcgttaaaagcaaataataataataataataataataataataataataataataataataatatatttatatatgctttaacaatgataactaacatttatcagaaaaaaaaaattaatatactctcaagctattttttaaaacaaataaaaattacatattataattatataatttctctatatttacactttttttagataaacaaattataaaaatgatgtaaataagacgtataatattattattttttacaacaacgtataatattatttataacgtagataatgcgtttttgatattttatatatatgtggtgtaattattttcttatccaataattagcaatcttatcttgcttttatccatgggagctaattaactaatggtaccaatcttttcattcttttaataattattctttccccactagaagactagaagagtatttgctaactacatatatcattgtgtaagagtctacaaatatatgtgaagtgaagctccacctgaatgcacccgaattagagagaagagagagagagagagagagttataataattagttgcttcaattcgatatatataaacatggcttacaacttccctgatgtgttttgctggattcagagtcttccaccaatctcagaatgggaaacaagttccatgtccttaaacatatgttcttcttcaagctcatcatgccaaccacgtcttaatctcaccgtatccaaaaacaatagcaataatcattcctcatcaaacctctattttgttattattgcagactgcaacattcccatccatctttggacctctaaacctttcaagccaagcagcaccactattactaataaaacccacaataataataagttaatagatgatgaagaaaccatttccaatctctttgttaatttcattcaagccatccttctttatggctccaacaaaaacagcactcccttcctcagatttccaaaccttgactccatcacttccaacaacttttcagatgttttcaatctttctttcttcaccctcttgttcctagtttgcatatatgaagctcctgctgcagattttcgttctgggtgtattagcaacttgaaagaccacttgacaggttttcagtcaagacaagcatcacataagattatgaaactcctagggtctaacttggaagagcattggatgcgttcattgaacctcgcagttactaactgggttggggaacttgaagcacataacaaccccttcagaacaccgtgccctttgttttcttatgcattttcaacaattgggttgtggaaggtgcagctttattgtcccctcttggtcatggatgttgagaattcaaaaagcaatccagctagcgagaggcttcaattctccctcagatatcaccatgtggaaggtgttcttcagttcaatcacaaagtcttgatcaaagaggagtgggctgaaatcatggtggacattgacaacataaggtaaccactaattaaccaccaatatattatcccattaagagataatacatgtggagccaataaataagcatcttaacaagacaaataaattagtcattaatcatgcccatatgttgttattagaagtttaggaaaaaattctcatcattagtacaatctttttttttttaatattttatacaatctttctcattagcttgaattctttaaaaagaggtactttttttttttacttttcttttctctaaattgttcaacttaactaaatttctaatagttcaagattttggtttagatttgaaataagtgaaattcgcttaaattttatattagaaaaaaaaacaacattgttccatttctttttataaaaattgacgatcaaaattacaaggttcttttatgaatcacattcatccctatatatttatttcgatatattgaagtgattaaccgaattaaataaggttatttgatccatatagccaacgttatattataggttttgttattgctgtatatatattgagcatttttttcttatacatgtttaaattaatttcctttatgaatggcaatcatctttatgtggttacatacttcgctatattaatcaagtcaaaatgatgttatgtgatccatctaaattactgttattattatataatgagataagattttgttattattatatattaagtgattgtttttcttaatcctttctaatttaatttcatatatatttaatttacattaatattaattaactccttgtggacaggtgtgatgttataaaattggtgaatgagtcccttatgagccaacgaggggtaggtgcagctgaaaaacattttccttcgagaatatcattgcaacttacaccaacactccaagaccaagtgttgagcctatcagttggcaagtcctcagagaaccctcgaaaagaaattggtgttgacaaaagcgtggaagcttcatttgaaccatccaatcccttggcacttaaggtgtcagctggagaatcctcaaccgttagtttgaagccttggaaattcgaggaatctgtttatggatacagtgcaaatttgaattggtttcttcatgatagtgttgatggcaaagaagttttctcatcaaagccttcaaagtttgcaatgcttaacccaaagtcatggttcaaaaaccgatactctagtgcttaccgacctttcaacaaggaaggaggggttatatttgctggtgatgaatatggagaaaaagtgtggtggaaagtggacaaaggagccatagggaaaacaatggagtgggagataaggggttggatatggttaacatattggcctaacaaacgtgtaacattctacaatgaaactaggaggttggagtttcgagaaatagtccatctcgacgttgcttagccttgcctcaagatcgtagttatgtgcaaactatttgtttaaacgcgtgattagttccttaattgtaagatagaacttagaagtgatcctttaattagtttctgcaggttgatatatgatcaatggatacgagtatatattattgtcaaacagactacttttaacaatttattttaaatatgtattctttatcattggcttaattaattgtatgtccatcccattaaatagactaatagattactcctaattccaggctcaaatagtattatgaaaaaaaaaaaattcatgtgctaaaaaagctgctatctacgacatatgttcaacaacggttacttaaaaatgtcgttatttaacattcaatggcattttggtaaatatgatatcattttaaagtcgggtttcataaaatcgtttttgaataatgtaaaataaaaacggtttttacgaagtcgtttttgtttgtgtttaaatttgaaattttttaaaaaattaaaagcgatgtgtcctttcgtttcgcatcctctcctcttctcgatacgcctctctctctctctctctccttattcatgtgcgtgtttagcacatccgtcggtctccattcttcaataaacaaaaaccagagagaggaagagaagaaagccttgatgggagaaatcaaggaggagtgcacgagcctcgacaagagcgagcaggagaaacagttgcatagcaaggtctcgaaggtggcgctatactcgagcggggagagcaaggcaatcgcgagtgcgtgcgaaacggcgtcatcatttggaagcgtgggccccaaagtgtcgcatctcaagtgggggaggtggtacaagctccgagaactcgaagtcgccactaatgggttgtgcgaagagaacgtcatcgacgaaggtggctacagaattgtgtaccatggcttgtttcccgatggcaccaagattgctggagacctggatcttatttgcccaaaggtattttttatcataggcgtagatgttttatttccctattttgctatttcaagaagtgaatattcttaaagatattttgtggtattgtaatgaggtatttggagcacctggaggctcacattatgctcattatcatttggttggaagaaggacggtatgaaattattcctttggttgttgatgttcatgttcttttagattaggaaattgtcaacaaggaaagaataacatgccgaattttatattgtaattaattggttatggccttgttggttgtcccatacaatggaagacttgaaaagatattaattgttttcatgaaaatatataatataggaagatgtgtagctaatggttgaaacagggcttgatgcctatagattttcgatttcatggtcaagattgttaccaagtactataatattgaacaagctcccctgcaaaataaatttgagaactcctaccttttagtttttgtagcctcctattttttcagaagcagctctggactattctacaactgcagttaagagattggaaattacgtgtttttggacatggctaccaaaaatccacccttcaaggtttgtggcattttcctctttatacatttcaaatcatgtacctacctacctacccataaacggtgcaaagtttattgataaataaataactgaataactgtgtgaaatgaactctatttttcatatcttgtaactccttaatggatgctttattacattcttataataatttcatgttgcaatgtcttttcatgtaattgataactaccctattttgttttgcctttttttttaagtaattgaaaaagataaagcaattaatgcaatctgagtttgttatctttcaatagataattctgggctcttcttcaaaggcccgtagagaaattcttgctgagatgggatatgagttcacagtaatggtatgtgttagtgttataatgtaattgcttcagttttgttaccattattcattagtttgatggaaagattgaaagagcttggcttttagactgcagacattgatgagaaaggtattaggagggaaaagccggaagatttggtaatggcattagttgaggcaaaggtatcaatgtcttcattggtttcctattatttattatttattttttaattatgtggatacaaatcttacaatcacttcctcttaggatgctgtggaataataactatttaagaaatcttacaatcacttccctcaaccttattggataatttctccccataatacatacataaataatataaatcccattatatctattttagagatgttcacaagtcaaagctctatgcagttacattacaacattgttgatagattaagttgcaaatttggtttattgttctcctatttggtaaattgtagttgtagtaatccaatttcctaggctctccatatgattttgggcatctttttcattttttgggatgtaaaggctccaaatacaaaaattttaaaaaaaattagcatttctacatcggttctggcatgaccgatgtagagatgctcagcattatacatcggtctttcagttgtgatcgatgtagaatgtttatcattttacatcggtctttcaacgaccgtcgtagaaaacttagcattctacatcgatcctgcagttgtgaccgtcatagaatgctgatttttctacatcggtcacaactgaaggaccgatgtagaatactgagcattctaagacgattcctggcaaccgtcgtagaataagtgatcacttttaacgttgtctcctacgatgacggtcgtaaatcgatgtagatagtctcctataaccgatgtagattgtcttttttctagtagtataacttttacaatatgattactttaaaagtcatatctagtaatatatgttgtcaaaatgaatccaaatttttaaatattagcgcaagtgagtaaaaaaaattaagaagtgcattccaaaaggtaaaatgaaatgtatcctttttgttttgaaactaaaaaaaaaaatgaaatgtatattggttcaatgattcttgccttcacggagaactctaactctactaacataatgaaaaggtaggtagagagatgatataattaaaattcatcaaataatatcataaatagagttattcaccttcattataaaaggaagttcttgagatgaaaataatcatctaacctaaacaaggagataccattacccaagtgtgtgtgtgtgagacagagagagatagagaatattatgaaggaatgagtttctcatatttttctagaaaacttctaatagggtttgttttcccaatagtgatatcaaagttggttggtgcactcaagtgactagtcaaacaagtattaaagcaagatgaagctaatattggatcaatgtattgaagttttcttagcgacgtagaactcaagtggtatgtcttattagtgtaagaagaatattcatggtgagtaggtgtttgtgaagatgctaggattgatgtttggaaattttaattgagagaattgaaggggttattaggaaaatcaatataattttcaaatactaatataagcgaggagtgaaaaatcaagaggtgcattttttgaagtaatgaaatgtattttgactcttgaagaatctattactataatgaaaaagtaggtagagagataggataatagaaatccactaaataatattcatgaatgagtcattcactatcactataaaaaaaattattgagataagaatcatcatgctcaaataggaattcacaaccaaagtgagggaagaggcttgtaataaagagtttttcaattttgtctctaaaacttgtaataagatttgtttcccaacaatatacttccccttgtctcaaatataagaaaaatatggcacatgataaaccataatttgagtgatattttatgtgagtttttgtgctatttcatgtattttcttaacaaaactcatatttggacattagtttttaccttacataagtacaattgttcaatcaagtgaaaaagttgaaaaatgcataaattgatgaatatttgatactattttcacttgactacgactgtgatacattgaccacagacgtgatcaatcaagaaggtcatggtcaatcatgagggcttcttcatttcacaacatctcaagcttcaacaccaagattctgataacgaccgtggtggatatcatcatgaccataatgagcttaattaaggaaattatctttaggagtctaattaaagtaattatctttaggaatctaatcacaatatttgtctatagtgcatcgaattaaagtgattatcttttgagatcatattagaatatttatctatagtgcgtctaattaaaataaatatgtttaagagtctataaataagaagttggagtctatacttgggagatttgaaattcattataccttttatgaataacaagagcttttgagaggaaaactacaccgagaacaatagaggttcaaatctgtaagggtttcaatctcttttatttctaatggaatattgtatgttgtttagttattctatgaccataattggctaaaccccttaaaactcggattgtgatgtagttatacccatgtactctagttcctctttttaataaagttctttgatgttatttggttaattgtatctttgtgtgtttgtgatcatcttcttataattaactgaccaatagaatgatagggtttataaagcttgcatgaccaaactagcggtatgaatcccatttttacaaacttttctgttattcatccttaatcctaaattaaatcacgaatgaacaatgcatggttgatttagggggaagagtattcttagatcttgactcataagatgttactaaaataagaggtagtagacaattaattggtaactgagagttcttaattaaaataagaattaggggaaaatgatacatatgaaatcataatcagagtcacttttattcatacatatctcttctttaaaattgttatttgtctgtagttttattttctaaacacaaacaatctccaactcaatataaaaatacaaaattatttagtttatgcaaattagattatttgggaacacaactagtctctagagtatgatatccagactttcaagtccactattgctattgtatagggtatacttgcctttaatgaggacatattttatccatcaacacactaactaaaaaagtaattttttttccaacttacctttgattagaacttgatatcaaggataagaaaaatatatgaaaactaaaacctcaattaaataaaggatatttaagagatagtaacattaaataagataaaattagttaaaattttcttatattagagaccagagggagtaattttttattacttcacggtataaaaaaaattataagataaatagtcatttttaatccctaaaagtgtagggcacagacaaatttgtccttaaaagatgaaaaattaaaatttagtctctggaagtgtaaaaagtacaacaaatttattctttcattaactttccttcgttaatgttaatggatctgcctacgtggcacattaggacgaaaatatcaaaaaatgtttgctcatgtggccttcattgcttggcactgtctttgtcgagattctaatatgtgttggtgcggattgggggccttaccctccatttctcagatctggaaatcacaacctccttttcttaaaccaaaaagaacacattggtgtcatcgacactctgcgacaaccaccctccgacgacattggtgtcatcaccgaccctaatctattaaaacaataaaaaaaccaatttgagatatgtacaaacaaaatgaaaaaaaaaatacaaataaataacaagatcaaaaccccaagtcaccactgtaggtgtgaccatgatcattctcagtttgctgctctcgccgtcaacaatcaccgatgttaagcgctttttctccttgcactaagccaccacttcctcatttatcatctccaaattttgtaacccttagtcgcttggtttgtcagttatttttatcaaaacactatctacgccaatagccttaatctcctcattatccttatcacagatttgaaacctttccaacctctaccaacaccaccatagatatgaaactccctaccaatttctttaagaacaagcccatagcaaagaaaaatccaagcttcaagcactaggtgataaaaggcccgacaataatggtgattgtgacttcgttgtggtttgcacagtccggccacaccctctatgtcgcttccatccttcgcttcgactacttggatctttgattttggctttcaatttttggtctcatatttttgttgtttggattttgggtctcgaattatgtgttatcctctattttggggtttcaattttaggtctcaaatttatgttgtttggattttgattgtttgcagattcaattatggttacaggaggaaggaagagacaccgtagataacaacgatgccattttgcaaaacctaattgacgatgaagtcatatttgtgttgtttatgtgaggagggtgttgttgaagtgaatggggtaattgacgagggtgtaagagaagataggtcgaagcttcaattgacgtttaattaagtacaaactttctattgtcaatttcatcctaatagacacgccgtcaacataggtgtcacgtagccacaatacttaacattaatggggaagaattaacgatgaaatagttctatcacactttttacactttgcgactaaattttatttttctttcaaagaaaaatttgtttttactttacattttcaggaataaaaataactatttatccaaatttatattaactacgtggaaactactaaattagactttttaaaactatttattattattattatgatgatgatagtcgaagaaacctaccctgcctcgaggggagggtcccacgtaaaaagaggaggcaagcactagtcacacacaggcgcacgttagcgaggaaatgttcctagattgaaacggagaaggtaattagaggggcggaaatctcaaagtgacacacctctttcctccttggcttaactcaattcaactcagctattctctcttctctataagttcctctcctccattgtttttccctcccattcttccatttctgttttctcccatccctctcgcgcgctctctctcccatggaatcctccgctgccattcgatcttttcactgtatctctctctcttaatctgtatctgcatttttgttatcaacgtgctttgccgagtgtgatctctgctcttgtgtttgcgatttctgatctcgccgtctcgcttttctattttatttgctttaatttatgtgacatatatgaggggaaatcaaaatgattgagtatatacactgtcttgttttctgttttatttgctttaatttacgtgattttttactttcgagctagagagactgaagtaatggcgaagaagctcgctagatttgtgattttgttacaaatatttaatgtttgttgcgtttaagctctttccgcattgtgtgatttcttgaaatttcattagttatatgaaatgttataatcattttgaattgaatgctcaacaacttaggatggagtttctgcttctgctgatttatttgtacttttttacgaggaaattgaaacgtgtctactgcaaaggattgtagaattttttatatatatatagatttgcacagaacaataacattgaaacgtttcatcccgaagcaactagttgtataagcttgcttgacttgttccttatttctgaaatgtacgaagcttaatttagtctactctgtatgcaaaaagagttgaaggataacaccaaagaaattaacagaaaaatgaggtagctgtgccaagttattttatgctaatgctaagtgttgcttatttatcagaatcacgagcataacattttcatgaaggcaattgaatgtatccttcatgtttctacaaatatataaccaaaggatgctctgcaatcacccaagagcatgtaattggcattctgttgggtcatatagtaggacacaagggactgttttttttttttcaaaaaaaattattatttatcaactttggtgatacccataggctgtatactattttttattggaaacacttgtcagaaatatttgtcttctgttggttattgttttcttttcttaatctatttctgttaaataaaacaataagttctgttctttttccctgccttttttaaataagcttacttctttttggaaagattcttaatgccttcttaaagggtcaattttcataaatcataatacaatatttttgtaatacttcaatgtttttttatatttttaaaggggcaattttaccattgcaaccagatgcttactcatgactcttggatttcagatcccataggcactatgtcccatgtgcgagcctcccttgagaagcaggctgtagttcccattcataatgccggatggaactctaaaagtaggcttttcatccagcatttggcatatggtcagaagcacattaattcccacacgaaggggaaaaacacactaatttcatgtggaaaaacagctgaagctatcaacgcatccaaatctgatggtgggttatattgattgtgaaatgaattatatccttatgtttttgttctatttggctattgtttgaatgagaaatgtctgatactattatttgttcttagtgtaattatctgcttagttataaattttaagtggttgtttttattcttaagattttgagatacattaacaagttgcaaaatttttgggctgcaatttcatatttttatatacatgatcaaagataatgttcctgaaccattatctacttgttggaaagaaagaagatagaagaaaatccatgcaaacaaaagatgtcatttttattgtttatctgtttatttttaaggtattgtgttatttacgtagatacgtaatagtaggaaatcattcttctctgttgctaaaaattattatggtaattgagaaagtctgtatgaaggttatatatatatatatatatatatatatatatatatatatatatatatatatatatatatagctttgttatagttggctattagaatagcttccctttaataaattttactttaacattcgtattactttacctaacaattatttatgatggtgcagcttcttcagataacactccacaaggctcattggagaaaaagcctttgcaaactgctacttttcctaatggatttgaggtctatagctattctttccttttatttacctgcaaatcatatttcttattagtgaaaaatgatagtttggtgggaattagcaatagtatactcatgaatacaaataccttatgttacctattagaagaaataaatacaaggtatttgttacttatgatagctggtttttatccaagactgcataattgcatttgttaacagccatgtttcaatgtgaatctgataaaccattacttatgtctggtttgcttattatcaggctttggtattagaggtctgtgatgagactgaaattgctgaactgaaagtaaaggtgaacttcttctctcttgtttactgcttatgtaaaacttgaagtgcagtattttaaatataataacaaggatgaaaagaatctctgagtgtatgttttaaagcaaaatgttatgtggtagggttctttttgcaattcgtaattagtggctggatatgtctatagcctgccatatactgtgtattgaacttatgtcaaaggatgattttaattcacttaacatatactggtatttatgtttttatcaaaatggtagcacattgtaattgaaacttgaaaatgtggtgcttttgtgcctaatgtcctatctgaatatataatttttcttggaattcaaatttgatcataaaaagttgtgtcatctcagctttgtttttacaccaaattgtgcttaataactttaatataacttcaaatctgtaatttctgtctggttcatcaaaagaagcatgcttttagctggatgaccttgtacacaaacttatttggcttatatattgttataggttggagattttgaaatgcatattaagcgaaacattggagcaacaaaggttcctttgtctaacatttcaccaactacaccaccacctattccaagtaaacctatggatgaatcagcacccggaagcctgccaccttcaccaccaaaatcatctccagaaaagaacaacccatttgcaaatgtttccaaagagaaatcaccaagattggcagcattggaggcttctggtaccaatacctatgtcttagtatcatctcccacggtatgtcatccatcaataatatatgttttaacttctgctttcactcattatctgacttgagctatgtagatcactaatctttctttctcattaggttggcttattccgaagaggtagaacagtgaaagggaagaagcaacctcctatctgtaaagaggtacatgtgatatgcctgtcaatatccataattcacttgatactccctccactggattctcattatgaactccaccattggattttggtgcatgtgtttgcggttcactaagattttgcagtgctgaaactaattgtttctagtttagagcccaactatgcactgtgagctgccccatgtaatactgcataacaatagttgcagtctatgcatcagcctgattgcttgtcggcctagtctcctgatttgaccttgctgacatgcataagttggcttttgaaataaatttaagtatttaactgaaaaaactggatgctataaaaaaagaaaaagctgacaataattaacaatgatatattttggaacaaaaaggaatattttgtaaacttgtcagtataatatactgttagaccaggccagcaggttgaaattgggcttagtcaaaacaaattcccaaagccttggaaagagaagagaatcaaccctgatgagcagagagttggtttggagggaattgccaggtaggtgcttagttggtgaggattgtgttatatgggaggggatatggaggagggggtgtgcgcattctcttgggtagctcttgggtgagaaagttcagtcaatctataaggggcttggtcctgtgtagtgttgaatggtatgacacaatgactagttggcttcttcctctttagctactatagtctgccctgtgttttagtgttttaatcatactaggtagccatttcttccaaattagtgataatttattgagtctatcagatacaaattaaatgttctttggttcccaaatatagattgacctatatttttctgctggtgtagttattaatatattttgtaacatacgattgcagcattaagttgtttgaggacctcactttcattgaactgactacaaattatatattcatttcctaattgaattctagggtgatgtaatcaaagaagggcaagtcatagggtatttggatcaatttggcactggacttcctattaaggtgatctgtctcagtttcagtaattctggttccttcctaaatatttactacttctgactagtttaatttttggaatcttgcagtctgatgtggctggagaagtgttgaaactacttgttgaggatggaggtataataatgtctattacctgttacaaaaatgcctagatttattgacatggtactaaagtaatacagggcttctggggaaagagtgatgatagcatactagcagtgctgttcatccaaaactgaattagttaatgcaatagtgtgcctcagcaataaaatatggcaatcctaataattctaaattctagtagtttttatttcaagtattttgaagcttagttgcaaggaacaagcaaattactggcaagtgataagcattaaaaatgaattttaccatcttggaacacttagtactagagcattttctgacctgtacttatgatactctagtatcatggatgcctaattgctttatggctagcatgctgttatcatattaaaacatggggatgttgggtttgatttgtttagtttgagctctgaagccttgatactccaaaaatggcaaagtttcgatgttgtatttgtatgcaacaccgatacgcataagatactcgtgtagttatccaattccaaagagtatcctctatttgtttttccaaaattttattgactggtatggttgagaacggctccagtactttgaataggaataaaattcactacagctttatcttttcctatgtatctttaaagttatagtaaatactaagcattactaggcaatcatatagattcaatacttcttttgagttttgagtaggacaaatgatgcacattttgacacaagttaggggaagattttgtaacatgttggaaaataatacattctggtatgagtaatggaaacatatatcactgtttatttgtttttgttttttgttattatcataggtattgtttttgtgaattatgatcatcgttcactgagatagttatgtattagtgaacgataatccatgtctcttaataaatattatgcatttgtataaagtatgtatgtctctatctatctatttgtctgtgtgtgtgtgcatttgtgttaatgcatctgagttgcattgtatcttgaattttaagaatttcctgtatgccctgaatttgggttgtaccaagtatctaggcttcatagagtttgagaaaacattttttgctttcatttcttgttttctcttataattagaaaaatatcttaaaacagaaaacaaagtggaaaataatatgacttggttgtaattattagtgtcaagcttataagaatcttggatccatactcatttaatcatcataccagacagtgcggatcttgaatcctgtatacctttttatctgatttgctaaatgatggaaggtaacaggatattcatgatagattaattttgctcattgtaatttacagtatatcggttttctagttagtcctgactattgactgaatggaaaaatattgtttagctcttaaggtcttaatttctgctatgcctttttattgatttattgattgcgaattccgtccttttgcaatatttaaacctattttggcgcatcaatgatggcagttacactcatccatagctgtgatgaatttcttactattttttgtttgcgaattacagagcctgttggttatggagaccctcttattgctgtgttgccatcttttcatgacatcaagtgagatagccttttcctgttaatgattaatcatgcggcattttttttattaggggagctattcgcgtattcgcaaaagtttttaataggttacaaataaatgtgactaaatatttggtacggaggttccttattgaaaatcatactttgtataccaattgcttaaattgtaccggataagctattctgagaccgcctcattatgtatcttacttgctggagtttgattactagcttgtccttttattttatttattttctcccaaaactatattgcagaacttgtttttgagatttggaaccagaaatagaaacattgcactattgcagtgctagaatatatagaaatactgtattctcaacaaaattatgatctaataatcattacttatcaccagattgtagcaaattcttagagtttccaatctattcagtcacattcccaaatggtaagggcagattgtgatgaatcatgttttaaggctaaaccatgagaaagtgagaaaccattgtatcccagaaaagggcatgtgaaatctgtttgtccctgcccacattttattttataacgcaaatcttgacccatcaaactaggtagtcaaacatgattattacatgtagcaagatatttgatgtgtgatttaaaacttgagtctcactatcaaacgtggatataatttgaagtttctcttactaactttgcattaaagcaaaattcataagttgaaaaggtttgataagattgatcatgaacttggggaccgaaaggaaaatgatacatatattattgctcaaatttagttaatcctaattttgatacgaactttaaattttaaatgttgattcaaataaattatttgatgcctttttcaccttaatttttaaatttttttattcctttaggctatgtttgacacatcattttatctaatttttaatattttttatcaattgaaaaaattgtttaattattcggtaaacaagttttttagtagtttttatcattttttgaaatattatttgaagtagtattttttaaaacgttaatttctaacttttatattgtattcttttttatccataaagtatttattaaattttcttattatcctttttaaatataacatgatttcattatttattttttcactcattcctctcacaaattttttatattatactatttgttttaatcaatgtactatttataataatttcaacattatacttcacaaaattgataatgataaatataaaaaaaatattattttttgaatattttttacattaaatgatttatttaactattatgctaattcatataataaaagtataatgtctacttaattgtatgtttttttaactttattttaaacttggtttaaaatattttaagacattatataagattaatagtataatacataatatgaaaaatgtaataactgaaactaaaaatgtaatgaaaaaaataatttacctgtggataaaaaattataaaagtttaaaattattaaatataaaattataatacatcaaaatatatttttatccataaaatgacattgaaatttatataaatatattattctactcggactcgataacttttgtatttggtggtcgatcaagtcagctactgatatcctaaatatctgggagatttaaataaggcaaaatggaaaatttatcccttagtcataagataaaaatataatcctaataccaaaaaatataagattgagattagataattcaatctctccctatataagagacgattgagacttaggtaaaacattttgatttcaactcatacattattactcatatatctctctacgcctctaacttgagcatcaaagtgtctttacaggtaccttcccatcgcaattcatggagaggcttatgaaagagcaccatcgtgagcaggtcttgttgaatatggtaagaacatttggcgtccaccgtgagaccaaggaaaaattttccttcaaccacatcatcatcattaatggtgacaaacaacgacaacacctccacaggcaaccaaagaccaccactggggtaggtggccaacttgtttggcatgccagccatgagaatccttgaaaatgagagcccaacaacaatggtgaaaatcttgtgtcaggtgcctggtgaacattccttgatcgttgccttgcaaagggagatgaatggcatgtagtagcagaatgcccaagagatgcaggacctccgaagggagaacatcgcccttagagaatagtaccaaaagattcaggaagacaacaacgtgcactcggtctctgcaaccagcacccatcaaactcatctgccagagaccgatgaagttgacatgacatggagggaacaaacttcatcagtcactcaataggtgcatagggtggagaatcaactccatcctttcattgatggcatcatggagacacggttaccacccaagtggaagatgctaatggtaaatagttacgatagactctcgaacctagatgaacatgtagatgcatttgccacccagatgaatctgttcactaacgacgatgccatcatgtgccgagtattcccgacaatcctaaagggagcagcactttgttggtacactcgtttatcgagaaattcaattagactaaacttatgtttggctcattgataaaaaaatcaaaactcgcggacgtctaaactaaagaccactatgcgtagttagctcaaaccacgaatgacttaactaaaaatttggttgtgaaaaagaccaaaagatccagttacttggctaaatcaaaagattgagttacttggctaaattcaaagaccgacactagcagtcgacttagaccacaaacaacttggctaaatttgtggctgtgaaaaagaccaaaaaatcgagttacttgactaaatccaaagatcgacactagcaggcaactcaaaacagaatttttttacttttataagctcttccagctaactctagagcttaggggcatatgttgtacttgaactcgataacccttatactcggtggtgggccgagttgactactcagatcctgaatatctaagatttaaataaggtaagagagaaacattatcccttaatcataagataaagatataatcctaataccataaattatcttttattagaagaaagataaaataattaccttatattttcctattttcattatctgaaaagaaaatattaagattagataatttaatctctcctcatataagaaaccattgagactcaggttaaacattctaattttaactcacatactattgcttgcatacctctttatgcctctaacctgagcgtgaggtgtctttgcaggtactcccaccacagttaatgaagagactcacaaaggagtaccaccgtaaacatatcttgtcaaatctagtaaaaacatatatctattttttatattttacatttttcaactatttcagtagacaattctaccgaattcttataatttaatacgttaacttaaaagctttccgctatcaactaattttaccaaaaatagtgttaatttattttatataataaaataaaattcacatgattaacaataaagacatcttctattatatttttttcttgaaataaatgataatatttcatcattatttgacaataatgatatttttatctcttatatattattttgttttaaaaattctctaaatttaaaaatattcataacacttacgatttctttatatcaacaaagagaaatgtatatataaattataggtaaagaggtacccaaactcatatacatccaaagtacattaaacacccccaccaagcatgttagtagccataagttatattgtatttagctgtataaatgacaaataaatgctgcatagccagtgctactaagaaaaccagccagtgagaatgtgcacctaagaccgtaaaagtgtaaatccctacccgaccattattattttatgggaactaccgttactaacccacggaaaaattgttgtttataattaaatcattactaagttacaataaatcaaatattactttaagttaaaaatatagtcaatttatactccagatggtcaatcacttcatttggttaatctggtataactttttttttttttttttactgaatttggttttagatggtgtcgtatacttgatttgtatatattttttatttttacttttgccccttaaatataatcaaattacaatagaagccttgtataggcaattaggcatatacggatattggattggcacctgtgtcacatctgactcagatgaattttatcttacatgaaaattccaaaagaatggaagatatggtaagggataataaatccacttggcgccgtatctgtaactgtatggacacgaatgcttcacgtagagctctaaatttgagaccttatatatactttcaatattatttattttttttgcgaccacgaaaagtaggaatttattattgaacgcagaaaaggaatgttgtatatttttatattttaaatctaggattttatgagaggcaaaaatacgaagaatctctccatatgttttctgatacactttctctgttatatttttttaataataattaaaatttattaaaacaattaatttatatggaaatatcattaaataaaatatgaaaattcacaaaaaatataagtttaataaatttaaattaataaaaaaatatttttaaaataatgttaaagaattagaatatattagtagtatcttttctaaaagcacatgaattccacctaaaaaattgataacttataatatcacgaactaaaattaatacttttctttttcgaaagatatcattgtcttttttatttcttaaatatttcatcatgagttgaatctataacctatgttattcgtgttttctgaaacaaattttaatacctcaaaaagattatttatcttttgacttgggatatccttgattgggaataaaaacatgtcttctttattaatttagtatttaatcatgttcaattgaaattgtctaggaagaaaagaatatttgaaagaaaaaacttttccaaaatacacaataataattttatataaaaaaatacataatgattaatacaatcttgtaaggctgaaataaatgttacatgttttgtgtggcagaaagaaagttgaaaaagtgatctgagaaaacttaaacctttgcggttgatgacaagccctgattcaagcgaaactaatgaagccccacactacggcttcttctttcgccactagtctcccgcacgtgccttgcttccgcggcaccactgccgcacgtgccactccatcggagcctcatcatgactccgccggaggcctcgaattccgacgcgtctcgacatccaaacgccgcctcatcaacctctccgtccgccacgctagtcgcgtcaccgcggcgtcgaatcccggcggttccgacggcgatggtgacactcgagctcggagctgccggcgcggcgtgctgatgacgccgttcttagtcgccggcgcgtcaatcctgctctcggcggcgacggcgacggcgagagcggatgagaaggcggcggaatcggctccggctccggcggcgccggaggagccgccgaagaagaaagaagaggaggaagtgataacgtcgaggatttacgacgcgacggtgataggagaaccgttggcgatagggaaagagaaaggaaaaatatgggagaagttgatgaatgctcgagtggtatatttaggtgaagcagagcaagttccggttcgagacgatagggaattggagcttgagattgtgaagaatttgcataggcgctgtttggagaaggaaaaacgattgtctctggctcttgaagttttccccgctaatcttcaggaaccgctcaatcagtatatggataagaagtataacaatgcctttcaaattctgattaatgcttagcctcttgcatcaacctacttgttaaacctcttcaaattcgattttaacataaaacttttaaatgttgttgatattgcggcaaatcgcgaacattgatgatgccaatcgcggtcgcggccaattaaaaaaccttgatgttgccgcccaaatcacggtcacataccgattttaaaaccgtatgacatcaataaaacgtgattttgcccgcaacattaagtttattcaagtgtctacaaccgcaattgtggccgtagaaagtcatgttagctaaattaacaaaatttcaatcaacaattatagaagagagaactttttgttggcaaccaatttctgtctggcaatcttttttctggctcactattttctgttaaactctgcagttctattgtcttagtcagaattactttccacaattctgcattacttgtactgaaacttgactttcatggcaggatagacggagacaccttgaagtcttacacgctacattggccgcctcaaagatggcaagagtatgaacctattctgagctactgtcacgaaaatggaattcgtcttgttgcttgtggtacaccactgaaggtatgcataaatttcatccaaactaatgttgatctttcaaatgtttattttgtctctgaaaattatggttgtggctataattaggagcaaggggaaaatgttagtgtagtagatacttttggtacttgccaaattaacttacaataggtgcaaaaggtaggttctgtaaaaagttgcccttaaggattttgattgtgttaggcctagaaaaatctaatctacaaaggacagaccctgagttggaaataaactcataatcctcaaagattccctcccatattaaactaaaagttaaacaaaacatttaaaaaaaattgaggatatgattatgaaaagttgcctcaagtggtttgatcatgtcaagatatgagaaggaaatataatctgttaaaaagggtagagggcaacccgagaaactttattgcaatttttggtttttgatagagctaaaagttttcaattgatccatctagcaaactccaccttgtaggattaggctcagttcttgtattgttaggtcaagacacataaagatgacaattaatacttgaacttttctcatcaacagatcttaagaactgtccaagcagaaggaattcgtgggcttacaaaggatgaacgtaaactatatgcacctccagctggttcaggcttcatatctggctttacgtctatctcacgcagatcttcagttgatagtactcaaaatttgtctattccttttggtccaagctcatacctttctgcacaagctagagtagttgatgagtattctatgtcccagattatcttgcaaaatgtgcttgatggaggggtcactggtatgttaatagttgtgactggtgcaagccatgttacatatggatctagaggaactggagtgccagcaagaatttcaggaaaaatacaaaagaaaaaccaagcagttatattacttgaccctgaaagacaattcattcgcagagaaggagaagttcctgttgctgattttttgtggtattctgctgccagaccctgtagtagaaattgctttgaccgtgctgagattgctcgggttatgaatgctgctgggcagaggagagatgccctcccacaggtaagccaacaattacagttactaatttgcttgactgttactcttcttgctccttagaccctccttccaatttttagccctttttgtcctctcattcctagtgggataagactttgttgttgttatgctactaggtgtcttgtgtcagtacctattattttttatttttttttgtatgcaattaactttggcatttcaggagcattttagagcccacttgtttcaacctttttcatgaaaaatcacttctaaaatttcttagtgtgtttgttcattttttttaataataaatcagaatctatctatatgattattttcagaagctactttgagtattagctgaaaaaaagtcaaaattataagtacttttttataataaaaagtatattttataattattgtaatgaaataaaataactacataaaatatgtaaaggaaagtactttttttaaaagcttaaacaaatgggtccttatctctttaattgatatctctgttttgcattttattgattgacataaattatgcagctcaacctactgctgtgggggatttcaattgaatgtttacaattttgtagtgctgtctttgtgctatctttattttcctttaaattcatattccttttgaggtcagattcattgcacagaaatatataatttaaggacttaaaaactttccaatgggtgaattgtgataatgaggtctttatttctgttttctttctcctaaaataaagtacctctattctataatttggcatgcattagttgcatacttgcatttgatgattaggtagttagtgtgtttctactttctagttctttttcctaggcatctgtttactcttttatgtgcatgccctagacaggatactgtgtgccgatattggttgtaaacataggaaatattttcctttctatttcaggatcttcaaaagggaattgatcttggtttagtatcaccagaggtattgcagaacttctttgatctagagcagtatcctctgatttcagaactcactcaccgtttccaggtaattttttatttgaaaacgaaacctttattctgcttttaatcgagataatttcatcactttgttcccttcatattcttacctatccatctggttcttttaatacagggattcagggaaagattgttggcagatcccaaattcttgcacagattagccatagaagaagctatatcgataacaactacattattggcacagtatgaaaagaggaaagaaaactttttccaagagattgactatgttattacggacaccgtcagaggatcagttgttgatttctttacagtgtggcttcctgcaccaactttgtcattcctttcatatgctgatgagatgaaagcacccgacaacattggttctctaatgggacttcttggctccatcccggacaatgcatttcaaaagaatccggcagggataaactggaatctcaatcataggattgcatcagttgtatttggtggtttaaaacttgctagtgtgggatttatttcaagcataggagctgtcgcttcatcaaattctctatatgcaattcgtaaagtctttaatccagcagttgtcactgaacaacggattatgaggtcaccaatactcaaaactgcagttatatatgcatgctttcttggaatatcagcaaatctccgttatcaggtatgtagagaacttgtctgttgcataaagttttctttgtaactaatatgtaacaatagacattatggaagattgaacctagtttctgtaggaaatgaaggcttctatattgtcaggttgatggtttatgtttcaatactcaatagttgattatgtgatgtcttgaattttttcctatatatattaaaaccggacagagaaatacaatttggaaattcatgtagacattttgaaagcaggacttgaccagctgtgtcttgaataactagttgtatactatgtttttcttaccacttaggcagtgcttgaagtagatggtgggtagcaagcataatagaaaagattggacagtaatgtggtatgcttttggatcctctacaatctattttgttcaaataaagggagtagaggggaggaacacctggtgtggttcacagggaaataaaagagaaaatatatatatatatatatatatatatatatatatatatattatttttttttattatatcctcataataggataaatcattaaggatagtttagtctaattagtgtaaaactttctcctctcccaaattctcctctccaaattgatagggggattttactcccctcctttctccctttccttccaaatctttgccctccttcccctccattattttccaaccaaatattatgttaataaaccctggatgccattaaaagtaatagtgaaaaaaaatactcagttcaataatatctattatatctgattttgtaattttcttgccctttctacttcatctgcacttcgctgaataattatttgaggctctttcttttttaaaattggattttgattcttctgtgactacgtctttggttatatcatattcaataaatatgcatccataatgtctgttcttttgtattaatacggaaattataatattttgttgagcagataattgctggggtagtggagcatcggctttctgaacagtttgcttctcaaacattctttgtaaatatgctttcttttgtagcacggacagtaaattcgtattggggaacccaggtcagtttttttattttttattatcttctatctgcttttgttcagtttcaatgctcaatgttcacaatctcttcagtaagcggatttcatttacttatgattgaacactacaatcggttatcttaacatctaatatttaacatgtggcttttaatgcacggccatgtcagcacatttattgaacattggatgttaagttaattgatagaccaaaattgcatgattaaaataattagagatcaaaattgtggaaaaaacaatagagggacaaaatttgtgatactgaaaaagtaagaagaccaaaagtataattaagccaaaaaatatagagcattttatgaaaagtacaacatgtaatgcattacttttcatttaatacattttatttttttcgcttaacaagagtcttaaggaatctcattagcatttctaataaggtcttgaggaattaataatagagttttttttattagaaatttgactatattatcctcttccaccgttattgctctttcattttagttagtgaaacaccaaaagacaaaaaagcaatgtcgtctctatgttctaaagtgacataatttgaaacagactaaaacctcaagaatcaagagtagcaattgttttaaaaccaatggagtataattcagttttatgtgaattattcatctgcacataaatttcaaaatttgactagtataattctgttttaaaatttgttattttcgtgaattgctgcagcaatggattgacctagcgcgctttactggcctacaagttagaaagacagagtcaccaacatcagatactccgaatcctgctgcaattttgtgcaatgaaacagaagaagccagcattgatgagatagaaaaataaatgcggtgaaattaggttgcaacattttttttttcttggtaattttgtattattcttgaaaagctggtaaaataggcacaaaggaataggttctggttttcatcacaatctggttttcttggtaattttatactattcttgtatacgtttttgatatttcccatttcatagtttggtgaatgtaatatttttatttttaaatgtaagtggaataaatagaggtgtctcaaatcgaaaccaacgcaagtactcttggctttaagggatatatatctcttggtaagtcttgttaaaaatgatataagaaatcagcagttgtacatcttatatagtattagcaatttatcatggaacaatgaactttctatagatttgcagtaacatttggttaaaaggtttacattatttctctataatccaaaagatgctctgtagatacattgattgaaaagaggttcatgtcaagaccccacaggatcacttagtacttggtagtgtactgatttatttatttggaaatgaagaggtagttgagatttggcttgaaattaagggaatccattcagagaaccaaactggacttaaatggttttaggcattggcggctaaatatatatatatatatatatatatatatatatatatatatatatattaagggaatacgaaaaaaaaaaaaaaaaatatatatatataactttgtgaaattacaaataatatgctcgggataagaacccgggaacatttttatcgcaagcgattgaacataaaccagtctctttggtttgttgtttgaacttcaatttggtactagaaaaccgaaatgtgagtaatttatgattggaactacaggaggaatttgataattaaagaaaatatcttataaaatatatatgcatcaactttcggttcttttatgaccctgctccccattcgacctttgtatcatcagcgtttctttgactttgtaaaaggcatgttcaaggttttccaactgcatatttttcacaattcacaattcatgagtctatcttgaggtacttggtttaatgctttaaaaaggaatttaaaagtaaataagcatttcgctggtagttccattacctcttgtctggcgaatctgttatacacgaataatccactgtacaaatcaaaactatttctcatcatagtctccttctgcaagtaactttccatatgttacataaccacatataaaggaaaacttttagaggaactgggggaaatgttgacaactctttaccaatttaacaagtgtctcaaagcccttagtgtcaataggtgtggattgaatatccatttctcccaatgtcctgatcaagaagcagccaaaagttgcatctagtgagcatttacattagtgcttttctattcgtttattagatattaaggaggctgcatacctgccaattgtgtgtgtgataaattggctcttagcagccgctctgtcatgttcctcacaggacatctgtaccatcttgcaaccctgcttaatacaaaagtaacaaaacacagtgaaatagatctaccagggatgagtcataaatgtaaggcattcataactgaattttgtgattcttttgtaaacccaagcaacaatggtctaaacttatgaagaatctattcgagctcgctccaggggagaatttaagattgaattgtatactaaacatttattttcagtaatgaccctgaggttctataaccttggctgtaggatcgtgtggttagattctttcaatttggactttaacctaatttaatctcacttgacttaggatatgagatttgctccccaagtatatacactactttagtcttacctatagtcgataatacaccccttcacgactagtattattggatttggtagtaagataagtgatctgttgataagccttgatacaatcttagatttgaatttaggcataatttaatctcataaaccaactttaaggtaaggtgatggttgtttctatttatatacactattttgaccattttgtctctaatagatgtgagactgatacttatatttttcccaatatagatatcatatatcttgatttatcaaatgcaaactcaacgtctaacgttacttagcttccaccctctctgtttcttccgtcttgattaacacaatttacctggcccaatgaaaaaaatgtttctttcttctttcgtcattgacgggtattgacaatattggacctgtttggatataggctaaaagttctttttgacaacttctaaaaatttctctatccgaaaaaattctatattttcagtaagaaaattcttaaaaacacttacaccttattcaaacaggcctaaaggcatttaacctacctcagtagcaaagatttggatgaaactagagcaagtagcttcgtctcttatccgaactttgtcatacatgaaagtgtgatctgcccatccattattggcagtctgaggaccaaacattgggtgcgtgcagagtatgtctgaatcctctggcaactctcgtagtagaagctctcttgggtgctctttcacagaaagaacatcaacaaagagcgttggtcgcttcagggaagtgagtggcattgaccctacaacctcggatagcgataatatcgatgtgcacaacactataacatctatgtctgcggcgaggaatgcgctgacatccctgtgcaatgtttgaaatatggatcatggaatgtctaagaatttgcctttagacataacttcacaagatagacttgttctttacttatatacattttttttattatattactggtcgattgtatgtgatggatcaagtatatatgtattcatgcatggtatggtttggtttgatttacctgaaaaaatggatccccatttggagacaaagttgagagtaatcagatcgagaagttgctgtgagagtgtggccttgtttaatcattgtcttggccagaaactggccaaagttgccgaatccaacaatgccaattttgaggctttgggaagaggatgaggttgacatggttgtcatagtttcaggaaaacaacagtatcagaggattttcgctgctcaattaggttgaaacttgaaagtaacgaggcctatttaatgctgctctgtccattgccatacacgcttgcgtcaggttcaaggcttaggttactagtaatttaatagtttgtctattcgtttttttttttttatttgtactttaaagctccaactcaggaactttaaatatagcatcttaattcttatgattcctcctcacacttttcttgttttagaaaaagaaaagggaaaaaaatacaattatcttctacgatatttacatgaacagatacggaagcctaataatactcagcaaaataaatgaacagatacggaagcctaataatactcagcaaaataaatgaacagaaataattgccgtaaaattttcactaaagtatgtataaaaaatgtactttaaaaataaaaatggtaattagagaagaagtaaatacgttttggaaagagtaaattactactccctctgtacaaaatgattgaacacaacaggaatattttgattgttgaaagtgtaacgcattagtaacttttacacaatatttctttggtctcattacaatttgagaaaaaaaaaatattattaaggccagataataaaatctataaatacattaataataatataagattaattttataaaattgatatttttttttcatttatttattatttttttagtctctataaaataatttaagaagataatcattttagtagttcccaacaatggtttcttaatctctaacttcttctaaaagctaatcaaagtgatgcgtaacttaaatttgttcttaaagagtaatgaacgaaaggtaacgaggataggacgatattaattacaagttaattattaaaacgggattacaagttttatattaatataagttatttattatgaatataaatataatttatatatttaaaaatatttatttattataaattaattttttaaatctatctattatatagtgggttaaaacttaaaatactatttttcttaaaaaaatagattgatggccacgtaacattgcgttagtgaaggtggtactttttaaattgtgaagttggtttagcgtgtcccaaaaaaacactttttaattgtcatttattttagttaaggaaggtacacgtacacgtctcagacacgacatgtatggtcggtcgacaacaaatattgcttcaagactttctcggccttctgaactttctttcgtcagaataaaaattagagtcaacacctaatttgattagatgctacaaaaatcaccggtctttggctcctataataattatattaaattttataatatagcaaagacatttaaaagattccttaaaaaagatctgatattatattaaattttatagtgtaataaagatatttaaaaaattctcttaaaaatcctcttaaaaattaatttataagaagtggtctacccaactatataagcatttatcatatatttatgaatcatccgatgggagactattcttaataaattatcttataacaataaaaaaaaatctacaaaaatcaccccacttaatttactctttgaacaaaaatatatcaaataattttttgaattaaagttataaatgtaaaacattttgattcttttttcattttaatttagatttttaagaattaatttgagtcactgatttttttttaagaattagactgatgcatatttttttacgactaatggtgcactaaaaaaaaaataataacaaagattaaaaaaatttaagatttttttaaactaaattaaatacttactataaaagaactaatataataaaaaagtgttaattaatgttcaataaacataagttaagaaattaaaaggaaaatctttattaaagaatattaggcgttgagggtggcgcgacatcagctcatgcatgatatttatatggtttgttctttctttaagagaaaaaaaaaatggttgtgtacgccatgctgcatgccactaactaagttttgacagacagagccactaactaagttgatgatttagaaagtgctactttgattaatgcatcccatgtgacattgtatggttttctgcccaattttttatctaaaaaattgtggcattaaatattaccgatttatgtaacaattttaggataacgagacaaaaactttctaatttgataaaatattttagaataactcctttctaataattaatttgcagatgtgtactaaactattaatttaattaaagttaaattattcatttgattcctttagtttcataatttttagttttttagtttctataatttgaaagtgatttttttaattcttatagtttacattttaattttcttttagtctatgtaggtttaaaagcgatttttttagttcttatagtttcatgattcttactttttaatctctagaattttcaaattatagggactaaaaaagaattaaaatacaaattataaggactaaaaaaagagaattaaaatgtaaactatagaaactaaaaaatcactaagaatcattaaattatagggatgaaatgagtaatttaacacttaattaatcaacttaatagaaaataagtgtatataatttttaaggtgagttttaagatgaggggtccaaaaagctcatgtgaagttaaaaataactgttttttaaggaatttaaaaataattgtttggtcatgatatacaggaaatgattcaattttagccatttatttatttatttatttaataatatatgcaatcgttatttttaatcccacataggtgagggacatttttggctcctcacctaataattcaccattttttaaaaagtttatttaacttagcactacacaacaccaaagtgtaagctagtttcctaaacaacttgattgtttcggctcaaaaagtgtgtttacgattcggtaagaaagaaatagctagatgaagcagatagaaacaaaaaagagagagaataaagtaattaataaaaataggtgagaaaatagagacagttttcattgattttcattgaaatgttttaatactatatatattacttattttaagacttaataacttgtatataagttttgatttattaatttatttagagacaattatttaactactaatgtaaaaggttatattattatgtattttaaagagacttttaagtcagacttttgaaagtagtctatatcaactatataatttaagcaagaaccttacttcttaaaaaaaaaatcaaactataaagtctaacctaatctgaccctctaataaataataataaaaaaactgattaagttagaatgcataagaagagtgacttttaattaagtcagaattttaaaactagtctatatcaactaaataaattaagctcgaacattgagtttttttaataaaaaaaatcaaattcatcatgttttataaagtctaacctaatctaacttaaccaatttccatccttcatcttcacgtagtttcgcacttcctcaacttttttttttgtataccttattattctcactaaaccctatgtgtatatttttatatcattatataaacttccatcttttccattatttaattaaggttttaggattattaacaacagttttcaaagtgattataataattttcactttaacaattagtacagtaaatatatatatatatatatatatatatatatatatatatatatatatatggagcgagtaaattaaagtaagaaataatttggaactaaagtgtaattttttttttttgccaatataatttattcattttttttatttggcgaatcggtattattcgacgagaacgatttaggttctcttcttgtgtacccctcaattacagcatgaaatggtacaaagtggaatcacattaagattttttcttcttcttttatttgacatgaatacgcaccataaattgacacataaaatagagatgctgcttcgtgacgccactatcttagtttacataatttcttgctttaatttaccaagaaattcaggtactttccttccaagtatagccttccatgatcattgctttatcgaaaaattggcttgcttcagcagcttagcatcttctaaaagactttttagtttacatattaaggaatccaccttgtgaagacgtgcagtttgaatctttctgtggaagagaattgataaattattttatgatttgagatttgagattattatgtatatataatttctgtaaatttacaaaacgtgtagttggattttttaatttacgaaaaaattaatattacgtgttacaaagagattaattgggattgtaaacatttgatgattctggatcattttttttttctgattagacttgtagcttaaggattttatctatattttcttttttctccttgttttgagttttattttagtagaattaaggggatttcctcgttacatgctcgtaaggttgaataccaagcaaagttggttacgcgtgtattttccaaaatgaaatcacattttcccatatctatgcctcttcgaacctttaaggaagttattagtaccatttccgtcttttttgtaattcaaaattattaagtgaggacaacgtattagtattaatcgtgaaatttgtgtttgttttcattaataaattattaaattgtaaaattgtatatattttttgaaaaaaaaaatatttttaagatagtaattttttttaatgaattgttctattaagaaataaatgtttaaaagtgagacataaattttttattatcagtcttttttagtcaaacaaataaatttttagtttttaatttaatggagaattatttaatatttaatgtaaattacgataataatttagataaatatattcattttagttataataaggttataattcataagataatagaataacttttgataaaaagtattttcaatttagtaattaatttgtaagtaattttgacaataataaaaaataaggattaaaaaagtaaaattttgagaagaaataaaaattaaaaaaagttaagggtgtagaaatgtacagtaaaactgacatgaattcataaaattagaaaactgaatacatacatgataatatattgaacatgtgctaattttgatgtaggcgtgttgatgttttgagaaattaaataattgtgtatcaattctctttattacaacatacaagttctatatttataaagtataaattataactactcataaccgctataaatttccattaaatcttcactagcaatgactttaatctttgatctctaccattgatgactttgtcttcaatttttaccgttgatgacttgggtattcgatctttacccttgataacttgtatcttcaatctccatcgttgatatcttccagtgtcttgatgctttttaaatcttcatttcttctatcaccacacaattagccaaaagattatattttgacacttagtctaaatagtctattaaaagactatttctcaatctttcgacgaagataatttttcagtaataaaaaaaatctcaatgttaacacaagcataatctcataggtatattcaaggaaggagacaattcttcctctaaaggccttttatcctcctggtaggctcttcgtcatgagttaatgttaaatttttatcaatatcaaagatggatcgttgaaacaaattataaataaaagagcgacgaggttcaaaaagaaatagatattgaaaaaaagtaaagtttcgagaaataacaaagaatgagaaaaaagtaaagggtttagaaaaaaataagaattggaatttaaatattggttgagaatattgttctaacatgtcaatccataaatgttgcgtaaacttatgagcgtcgcgtgaacttaaaaagatacgaggatgatacatggttcaacatttattacaatgatacaaattggtatttacagacaataattcataattactcataacttctcaaataccttatagtaacatcatcttcaccattaatgatttttgtttttgatcttcacccttgataacttgtatcttaaatctttattgttgatatcttttattgtcttgttgtttttcaaatcttcattttttctatcaccatataatttgtcaaaaaattatattttgacacttactcaaaatagtttattaaaaaaataatttttaaatattttatcgaagaatatttttggtatgttaataattagtattaagtatcatccatatgtgatcaattttcttttttcattgctttgtcgaaagtaacattgtggatttaatatatctacactctgtcatacttgtccactgtatcatttggaatatcatatacgagctgatctctgtgattaggcactggattcgcattgacattgtgtttaacaagcgtccctcttattctctgttgttgcacaaccaacaacgatgccttggtcacaaaatctggtgatgctagcatgtccccaattacccctagttcttggcactcactccattgatcatgtccctgaaaaataacactgtctggatgctcccttcccaccatcactagatcaaaataatctatcaatcttctaatacatgttgacatctctatcccatttttcaccacttcattcataagctcaaacctttgatttcccgcattgtagtatctatactcatctataagatcactgtcacgttttctatctttagaattttcatgtccaaataaaaggaaccttaccacagttacatacacacattcatgcctaaccattctggaagcataggctaatgtctcggcgtcatcttggccgccgatgaagaacacagcgacatagaatgctgctctagccatcaagagtgaaggagaaggactcaagatgcccctatcaactagaattccaacagaacatggtgccctttcgaggacttcaatgttcatggtttggatggtcctatgagaaatctcaacggtggcatcaatttcccacctcttgtggaatggcaagatcaaaatgttggatccagtgtctagtgagattttacaaatatcatcatacatggtttcaaaggtggagattgaagtgaaggattgaacagatacatatccttcgttctgttgtgcgtattgcctcaatgcattgtcaatgtgacttgcattgcaagacattgagcgcatctcatcatggggttgttcttgattagcaaaaaggataggcctggctcttccttgaagctccactaggactaatgctgttacctcaatcctgctctctctgcttgcataggatgcttctaagaggttcaggatcatggggagattttcgttgttgtgaatgcacaccatcactcggagctccaaatctcgcctagtatgctgaattgtgcatcttcttccggtctgatatagttctgaaggatcatatatgtatcttatgagaggtactaggatacaatttactactataatcgaggccaccatcaaagcgaactcttgttccgatattaactgccatagacaacaagttttagactacgttagttagttatttagttaatcaaccaagggtaaaatgtccctcaattaacattggttttaatttattaaatttatagtgcagcataggcatttgaaaggtccctttaaaaaccagtttataaggggatggcctacccagctatataagcactcatcatgtttgttaaacatccgatgtgagattattctcgacataatccatatccatacactttaaaaatgataattttggtttttcgtattgaaattaagttcactattctcgatctcatatgcacatgcatacactaaaagggtctaactcagttaattgatctgagtgtataagttattgtaaaactcctaacactatctttaatttccatggataaaagaaaaagatgcacatacacaccttgcctcttttccacatattgtaagtggcaagctcagctataccccttccatttagaaagaggccaatgacaaaacattgtttcaagggtagattgtaatagtatcctggcaacatgacagcaccaatctttacaaagaaagcgacaatcagaatgacacacacaatccacagagactgcttattaattttgaagaaatcagtttgcagtccattaacagcgagaaagattggataaagaaatgccgtacagattgtctccaatttactcatcaaagctgttcctatgggtggcccttctggaacagccaagcctaaaagtataggtcccattataaaatgctgcccaattaactcactgacaaatgcagataagagaactaagagaaagatgcaaacaaggcagatttcattaaccgaacctcgacctgggtgctttaccatccaaattattgttggcctcattacaaatataacaagaagccagactccaactatggacagaaggatgcatgctagtctcacaaagctaccactctgatcctgcagtattgcaaacagaatcactgtcaagataaagccagctacatcagcaaacattgctgctgacatcgttaagcgtcctatatcagtgttaaggactttgagatcggtcaagagcactgctatgctaatgaacacagttaaggtttgcgacatagctagaaatggcagtgcctttgcgaggctcccgtccatcgcaatgtatttcatcatcaggaaggccagtccagtagggattatcaatgtaaatgcgaatacagaaataccaagagtgatggccaccttttcagtcttcattaatgtggcaatatccattttaacacaccatatgaagaagaagaacataagaccaaacaatgaaactgtgtcaagcactacagcgcccttcataggaaataaggctagccccagaatctttttgttccccaacattgatggcccaaacagcactccaccctgcaatgttaccatgcatcatttcttaacactggttcttgtagcaagcaatgaactttattttcaattgatacacgaataacatttttttcccattattggtggtaggagtggcagcaacacacactatcacaataattattacatggtcctgtcaatcttcatgtgcctttattgtaaattggaaatttcgattcagtgtcttgtgaagatggactgaaccatggtcccatggtgaccgaggaccatgtaatattttctcttctaacacactttttattattggttaaatttattagatactacaaaagcatgagtcaagctcattgaataatatgcatgcaattttttgttgcttttgtaagtgtcattaatcactatatttagaattagatggttatagagcattgattatagtgaccatcattgattaaacaccttgtaaaatttatgagcttaagagaaagcttaccataatctgggatacaatggaggattgacctagtgacttgagacatgcgtccataaactgtgtgaccatggtcatcaaagagacctgcatatagagaagacaactgggggattctaatgggttgcctcctttccagatatcaaggtgaccaaaactgtggggatcttggcataccattgacttgtttccaatttcagatgcagcggccatatttgtgaaccaatatcaagggagaaaaaacaaaccttagttgttattgtagttattgttaaataaaagtacttttccttgagaaaaaaaaaaaaaacaagtatacacgagagaataagaggttgaccttgtattttgcattttacatatttaacggtttgccttcaagtaaggtggttatagcagccgtttactcttgcaaagtcatggaatatatatatctataattatacaacttgattttggaggatacaaatgatattaactagagaccttttagcaaatccacagaactatgtaaaatgaaaaagctattgcacatttgccatagaaatgaatcgtgattattagttaatattgtcaatttacattgcttagaaatctagagtccacgcttacttgagctagattatctgaaaaggaaattaagggacgtgtaagtgtaaccttgtttgcagcatggtcatagatggatccaacgtctcaaatccaacatttctagatagcaatcagagactgcgacttttaccttcttaatatcatccattttcattttcgtgtggtgcagatcatgttactttttgcttctttcatttttatccccacttgtatgcataataatccgaatataacattaaataaattttaattttcatgtgctgttaattatttatattatcaattaattaaaaattagttgataagtagaactctaaattaaatttctcaaataaaagacaagacttaattatatgcagtttagtgttgtttttcattaaaattaaaatagagatttcatgtcagtaatttacataataaaagctcacattaaaagtaagcagtgaaacttcagaacagctaaacaacggcataatcaatcatccaatggttcaaatatatatatatatcacaatatatagttcaactattcacaatatatagattatagtatacaaggataggtttcgaggggagaaaaagtgtactattctaaacaagttcacttatataagggggttaattcaggaacttgagcagggtaggtgtgcgagtgttgtaaaacgtgttgtaaaacatctgatacagtgttcaattcccctacggacaaaaaaaaaattcacttattggtttgagcatcttcccaggcagttgcattcttgccaatcttttgaaggactctgatcacttgttctgttgtgacgtttcccgtaaccatgaccttgttcagctgtgtgtccacgttatacgtttcaatatctgcaaagaacacaagttttaaactacttaacacagtcaaggagtctaattcaacttattgaacaaggtgaattattgtaagctgttgacactatcttcaatacccagttaccacaataaaaagaaaaaactaattaacacaaaaatatgattgtgtttttggtgatgggaaaattaagaaccttcaattttcttgatggccttgaggattttcttgatacaatcttcacagtgcaaaccgactttcacttccacaacctgatggtaacatgcaacaagaaaaaccttctcagtctcatagttgcaaacagaaaacatgaagttagtaaaagccttacatttgccatgaggagaagcagaagaggtgtatgtaaaactccaaggcttggacaaagatgagaagtggaagagaaagagacaaaagaaaaaaagagaagtggggttctaatgatttatggaaccgcgtgaaatggagcaaatacaagatggaattggattcttaacgtgaattatggaagcttttactttctgtaagaaatatttcgaacaaaaggtgcaactttattcctattttaaggtttgggacttgggagtgtctggattcccctaattagcaattgaggtcggcaagcatcatgcttcgcctcaaaacaggataggaatatgtaatgaaacaataaggaaatctttatcttcaattatcataaaatccagcgtgattactattacattattattatgcgtgtatgcaccagcatctttaattcataattgattaaataagttatttatcttaattttatgatattttaatttcaagagtagctttaagtatttaatataaaaaatattttaatgttaaaattgttaaaataagtgtttaatttaattttacgggtcttataatacttaaaaatatatattaattttaaacaactcatgtaaacatcttcaatagagatgttttaagtgtgtgcatagctaaaacgcacaactctattcaatgattttaatattttgtaattgtgaaaatctttcattaattttttttaagtttgcttttgtactattttatttagttattaattgagtttcacataatagattataaaatttatcagaacagtaaaatgatctttttatttgttttatcttaaatgaatgaattttaagcataattttttagttattttctttattgatggggttagtttagtttaggaaaaaattatgccctttagtttattaatcatttgaattagataagtttcctttttgggtttatatattatatataccgttactataaacatttgtaatattgactattttcatgaagacaagttttcttcatttatctgatatttgagttagcgacacccattcacaaaaattacccaagtcaattattaatatgaattcttctacagtgtcatgtccacatgtgtgatatttattacttacaaagcaccctttacctaataagagataaaaaagcaaattaagaatttaggaaaaaaggcaatagcacaacgtaaaatgccatcacagcctagacttctcttaattccattagttggtcctgattgtcaacaaccatgatagcatgcaagctttctatttgtaaaacctaaaccagaaagtgattatgcatttgttatcaagagcaattatatgtcacagtttttatggtaactaaatacagtgtgatcctattaattgactcaaagacacaagacacaacactgaatgcacactatcaattgtacttggcaacttaatatactatatactgattgcagcaacacccaaatacactagataatagtacgggcttgtttggttaaaaatcttaattaagcacttattaattaagtgtttatcatacaagcacttttgtacaagttatttctaaaattgaagagaaaataatgctgaattgttttaatataagttgtacggtattttcataagctattcaggacttacagaaataagttaaaagcaacttatgaactgatcataaactaattttataagctttcacaaacatttacacaagaaattatatcatgagataattccaaataagttgtaaataagttcatccaaatgcatccaaagtgttaattgattttgacacgagatacacaaaatagatgtcagtgcccagctgcctgaccttttaagccttcagattaatggaatcatatcacactaaatttaaggttaaatagttcaagtattattcgagtttgattcttaacgaaaataatttttgaacagactttatttatctttcgactaaactttgaattaatcagtctctttttcctaatgaatcagaggattaagacaaaaaaaatatctaagatactggtaatacttctagactgctgctagttagtgaatgtttagcaaatggttatttttgggaaatgttcgcttttcttttataaaaactctcttaggaagctgataaaaaaaagtgattattttttcaaaatgaacttaatcgaacacatttggtttcaggcagcttatcatactaattgcagtgatgccaaagtaaactagatgttagttagtaactattgaactacaagtgtgaggtgaaaactcatattatgtaggaataggaaggttgaacaccatataagtgaaggaaagacccataaacttgagttttaaggttttgggttaaagtgtgatatcaagttcacttacatgattactcatgcctcattggtataaatctcctcgttgtttatcctcctcgattgcacaaaattgatatcgatttaggcagtgcaagtaactagagcaagaggggaagggttatcatgtggaagtaactcacatctgagggaaagatggctggaaaagattgccggaatataagtgtgatgttaaatcagacatcagactattagaaaataacatctgtagctatgatctcacatagaatagaggagaatggattaaagcctctgtgcaagagttactctcgaagtcctagatgcagagtggtcaatgctaggagcatatcaatttgaagttaaagggggaaaggaagtacagaacacaatacttacctaggcataataaataaatcatacaataaggcacacagttgatgtttttgttagaaaataaaaccaaaatgaaggaaaataaatatgaagaagatgcatagtcttgaattaaataacaaaataacaataacaaattaaatttaattgatagcacataaataatgcattatatcatacaatgagcacaaggaaaaattaaatcaagggaaagaattaaatagcctgggttgaagcgcgtaaacgctatccttagagagaaaacgcccccactgcacgggtaagaaattctgattgcgctcctctcccaagatacgacaacccgttggttccgatcgtgtgcagcgaaaggatccccgaaccttatgaacaccaatgctcttgctctcacaaaaattaagttttgtataggaaaagaaagagataaatttttggcagaaagaaaccagagctctcagtctgtcatttctagaaaagaggagatagatatatataggcattttgcaacaacaaaatatgaccgttagaaatatgaccgttggaaaaccaacatacagttgtcacaacaaatataacaaactagtttgactcattaaaacaaaatcttaagagaatctaaaataaatattattttataaaatagaattaatatatatttataaattttaaattaaaacacaaatatttatcaacattcccccacataatttaaaattttaaagtatattttctaaaagataatttgtataaaaacataagagaaagagcatgtgatattgtatttcggtataaggaaccttccgggtttgagccttatacctagtgtttatgaacttccatccgagaaaaatgtagtgacttgattgtcttgaactacatattctttaaccggactttagtacacaacccctacaatattggtgttcaattaggttctaatcagtggtagcgcgttacacggccttgcgcttgtatcttgtttcgtgagtgtcacttagagattagcccatatctcacagtggcggccccaccaccacactcactaggtgaatcctcaaagagtgagttgtgaccctcacccctacaataattgtcatcggattcattaagaggtattttgttttttttttataccaaaaatacacatatataaatacctcaaccttaatattgccacaatttataatacacctcgtcataggaatgagaaacggaacaactccgccaaatttcattttggtgtactttagtcaacaaacaatttcgttgttacccgttgaactccattcatgggatcaccaatcacacggagacgggtgtccattgttgtaactaaataatggactttaatctcattcccctcgacgactcaaatacttgttgacatgtcaacctttttgtaagcggatccgcaatattattttttgacctgacaaagtcaagagaaatgacatcatgagaaatcaaatttcttatagacttatgtctcactcttaagtgtcttcttttttcattaaaattttgctagtccctttagatatagcaatttgattatcacaatgcattggaattggaggtataggcttattcaacaatggtagatcacatgatactgcaccactagtagcagtatctaaatcaataatttctacttccatagtagaacgtgaaataatagtttatttagcagatttccatgatactcacaaccagctaaagcaaaaacataaccacttgtcaattttatttcatcaaaattagaacttcaagtttgtatcactaaatctctcaattattttccaatctaccaattgcatgtgcaatatcagacaggcctagaaaagtttatcaaatgcaacaaagaaccgatactttgagaatatttatgtgaagaaattcctttactcaaatttttctttaacttgatggatgagtcataaggagtaaaaacatgtttcgcatcaaaataattaaacttcttcaatagcttttcaacataataagattgggtaaaaaatcataccatcatttttctctataagcttaatacccaaaatcacatctacacaaccaaggtctttcatatcaaaatttctagacaataaaaacttcacatcatttatgaaattcatttactaccaactatcaatatgtcatctagatatataaaatgacgcatccattatcatcaaattgtttcacatacacacatttatcactaacattaatttgaaaatcatacaaaagaataacttaatcaaacttttgtgtcaatgctttggagcttgtttcaaaccatacaaagatttaacaatttatttttcaagaaaaaatattttcaaagaaagtcacatctctagactccataatagtaccattagaaatttcagatacttctgaattaataactaagaatctataagtagtattatgtaaaaaatatccaacaaaatataattaatattttttttaattttccttttcttattaatagggatattaacctttactagacacccccacactttaagatatttcggatttggttctctttttctccatagctcataaagatttttcttttgtttataaggtactcttttaagaatacgacatgcaaaatataaagtttcaccaaagtgtttattttattatttttgtgtgttatattttcacaggcttatcttttatcaatgagttataaataaagagacaatcagtcaacatgtaacaacaaaatacttgcagtagaaataataacattaaacaataaaaattaaaaaccaaacaacaaatgtcctaattttaaagacttgtgttcacaggatcatttgaccaagtaaaagatagttttctaatcatataggaatgaaattagaagtatgcttttagtttttcacataaactaattctaaaagcattttctcttcaaaaccatcattaatgaagaaaatatattttaaatttcaaattataagaaaataattttcaacaatctctcacttatgtaaaatttaaggaaatgaaataatataataaaacatttttaataaaacataatacattgtgtcttcatccattaattttcgagacttactaaaaagggagtcaatcatattcatgacagatattttggtaaaatagaatgctactgtaaaaaagagactatgcaagaagaaagtgataacgattttctctctaagactgttggaaaataaaaccaaaatgaaggaaaataaatatgaagaagatgcatagtcttgaattaaataacaaaataacaataacaaattaaatttaattgatagcacataaataatgcattatatcatacaatgagcacaaggaaaaattaaatcaagggaaagaattaaatagcctgggttgaagcgcgtaaacgctatccttagagagaaaacgcccccactgcacgggtaagaaattctgattgcgctcctctcccaagatacgacaacccgttggttccgatcgtgtgcagcgaaaggatccccgaaccttatgaacaccaatgctcttgctctcacaaaaattaagttttgtataggaaaagaaagagataaatttttggcagaaagaaaccagagctctcagtctgtcatttctagaaaagaggagatagatatatataggcattttgcaacaacaaaatatgaccgttagaaatatgaccgttgaaaaaccaacatacagttgtcacaacaaatataacaaactagtttgactcattaaaacaaaatcttaagagaatctaaaataaatattattttataaaatagaattaatatatatttataaattttaaattaaaacacaaatatttatcaacagttttaagttattatatcataacatcttcctgaacaatgaacaacacaataacaactaattcaaaaatagagacatcattgttgaacaattgtcagttaacaaaatttgacttatgctttggatgttacaataagttcacatatactatactctagaattgaaattgaaatttaatctctttgaagaaacacgagttatacttgcaaattaaggaaaagattcatcttagttctccaattcatgaatcagaatcgcttgctctgtatcctgaatgtcacaaaatagagatcaatcaggacacataaaaccgcaattctttagtcaaacaaaaggtagagcttacctggccgtttaccgcacactttccaaactgaatttcgatttcttgatgctttactaatccaaacatgcccctataaacaaagtttgttacagtatagacagtaacctgaagttgagaagcagctaatagcataccaattacataattcaaattaactgcagtcatgggtcttgatgccctaagagtcccatccaccaatgcaatctaaggttttgtttggataaacttctccataagcatttataggaataaggaaaaatgaaataagtttcttccataatacaaaagaacttctacaagttagcaaattagctcatgtataaaagaagcttatttcatttgcctatgaagaaatttatccacacaagtcctaaggaaaaatagaatcacaattaaaaattcaaaacaggcaatggaattcgcccatatatttttttttaagtaaatatcaacaatctgataaaagttataatatatatgagtctattttttttatatcggcacacgttcgtttgttagtcttgttagaattatcattcgaacccacgacctctttttctcccttctcctttaaccattcctcaatcactaaaccaaacttatatcttcacatgagtctatgaccataccctttagaaggggtgtgcaatcaattatcaattataatattatacatagattgaaaaaaaagaagaagaaaggaaagaccttatgaggatcccttggaacaccatatccagagtaatacatttggccaaccaaaacctgcatggcagagtccccagccttggcctccttgagggtgtcctggaaccagcgcttggagcaatctgcgacaaccttggcgaggggtgtccgacattccaaaggttccatctttttcgcaggttcgaccttaacaccaattgggttcgcaattcgaggagtggcaacacggttgggcgatctgggtagcgttccctttgacctattgggcttcgcagcagatgacacaatccgcgatagctcttccaacctcgccgcagattcaagatattttcccatcaaatcaaacctcacacacaacaaataaaagtgtggcttttaagttcagatatttcagggcttttttatttttaagtggacgatgttgttggcgaagacaaagatcactatcctcagacctcaatctagcttgaaagtcgaaatggttagagttagttagaccttagaacaaaggtgtgtgtaattgtgcatggtatgtgttaagtgttacctgtggtaagaggaagatgatcaattgatacaaacaacacacttaggtcaaaaattgaaaggcctaggataattgttggcaatgacaaagattgcaaatctaccaataaaatggtgtatttttttttttcaagaaaagtaaaaggtgtatgcagtcacaggtagttacactattatttaaataagttattttcataataattacttacggttatatcttattatactttttattaattgttgatgtaacttttttatatatattaacgacgtatgaaaattaattttattttctatatttttttacagaatttattttttcttcctactttatctaaatatttgctttataaaatgttgcgctatttaaaaggtagataaacattattaaaatctactgggttataaaaaataggaaaaataaataatatttttaatgtaaatatcataaatataatacataaaaataattaatgaaaaattttaaacataaatatattcatttattgaattatcattcaaatcatcactaatcaaaattattaaattaagtaattaatcatatataaaaattgatttgattcgaacaaagtcattaacttttataatttatttaaaaagttagaaaacaaaaggttaagaaatatgtacatgaaaaataaaagtaattaaataaagtaataaaacaaaaataattctaaaactgttgtgatacttttagggaaaatatttccatactacatatttcacatttttcttcctcttaaaaaaatacgtttggaccgcagcaccctctatagaaaaatcaatgcatattcatatatttttaatcattataaataagattataattctataatgaaacttaaaatatctttacaatctttggtcagttagattgattatgggactaattaatagtccaaaaaatcacatttaaaatctttttttttctttttaaacatgtattctacaaaaattaaacatagattattcttcgtaaatttggcatatgttatcaagtaaattatacaatacatattcgtatcaataacaatacaagtctcctgtttcgatcaagcatcttttccatgttcaattaaaagtctttttctagttcagccattatcctttgatatcctttggagttatgagttcctgaattaccagtgctaccattcattccttcgtcatgtagctcagaattattttttgaccatctaacaaaccatttggtataataatatgcagatatgcgcttggttggcctatctatttttttacatagattccttctctattttggtttatcagtagctgaatacaaagattccttctctattttaacaatacaagtcaagcttgactttttacaatttgattttttttataaatttaataagggttaagaatcttgacttctgtactttgattaatggatttatttacggttcatgtgtctgcgttttgattcattttaagaatcttgtccttcgtacttttgattttgataagtcattccaaaaggaatcctttacgtttacgtttgttttttcgggaatgcaatttgaaatgtcaatcatcatgagatgagattttacgtagaaattgctgtgtatgaaacttggtttataaaatggtgcttgggctctgatttttttcacatgctctcggccatttcaccccatcttccactctcggtagaaagatatttgacagcgtgagagatatttttcctgcaaaacaaacctggaaaatgaaagtgagcgttgttgataattgataattgttgcatacgtccatttcaattataaataacacagtaattatttatttttttctaaagttttattttagtttagtctcaatttaattagaaaatgtaattttatagaatcttaggctgtaaataaaattataattcatttctcctaatttttacttttcttttcaaagatgttagaacatcaattgaagatgtcatacacaatgtttggcccaacgagttatgttggtccaaccagattaattgtgcaaacaaaatcacatagcaattcaagagtaaagaaaaatgtcaagtgatcaggagaacttgtccaatgagaaaaaattcaactagtgagtaaagatcacaaagtgaagaaaaaatattcaaaatatcaattacaaagaatttgcctagccactggaatcacatttgaagtgtcttttataattcttaaaaaaacttgctcattgagtagattatactttgaagtgacaagataaagattttgaatcccatataaaaggacattatcttaggattcccaagggaggcataaacctagaattgtgtgtggtgaatggaactctcatctactttcatcttccattttcatccattccccttcattttctcctttccatcttgtaattttgaggcttctcatgacaatgagatactagttcatcaattattgggggattggtatattaagttactcccatgtaattaatctttatatttattcagtgtcatttcctatgttattgcttttctaccattttgattgcttgttttatggtttgattatcttagcttcaattctttatttttattgttactggaaaatgcttgaaaattgggttttgaacaaaacatctaatgaatgtcttatctagggatggagggatgcttgttagtatcattactaatcaaccatatgaaaatgttttatagttaaactccctttggaaaatgttttttgattaagatttgaaagtggtatctaatgggtgttatgtctagggatggaataacatttgttggccttgataaacttttgtttttgaattcaaatggtttaatttgacttgtaaaggaattaggagttgagttaaataacttaaattattaacttagtgtagtcaaattcccaaaactttatttaatttgtatttggttccttttatccaccaactgtttatgtttttgctcaccttttatttgttgtatttttattgtttttaatcgtcttataccaaaccccaattatcctttgcctaattgtgtaatttacaccaatagtcaaatacacacaaaatcatatgaatacgatactcaaactttttattttatactatttacaagatactttgatagcttaccaaagattctaacatttatccatttgtggaagatgattgatgtcaattagtcaaacaaatctttagcggcagagatggttctcattgattcggcagaaaagtttctttctttgccttttcccaatttttgcatatatttgcgtttcttattgtgttggatattaatttgacgtgtaggcctctaaaattcaagccactatcagaaaatcgatgctcagaaaactttcgagttcaattaaggaaggtgaagtttgaggatattgagaacagtaatgggttctattttttacatgataatattattatttattaaagttatcattaatttggttttagttttggtattttccttttattctgataccaaaggagctcatcccaataataaatatgcatgcatattcctatattaacttaataacttcaccgtcatctttgtaagaaaaaaataaactttacaatcataagctgatttcattcaacccctcgtgtaccacaatttttcaatatcgagcactacaccagtttgatgatttcattttcaaaccacactcagtaaaaggaaaatccttatcacgtaaaggagaattttgtgatatttatttgtttaatattttatttaattaataatgattatcattacttttttagttgtcaatggctcatcacgccttttattttgttttttttttcgaggggaaattacagttgaacaagtcattagcaacatcatcaacctaactttttcctattcccccttaatggtcatttgttgggctaattataaaaagaactatttacttctcatggtcatggatgggcaattgcttcctggaccctaatccagaaatgtaatttacattcaggattaaccatttcataacgtaaaaaaattatattctggattacatttcggaaaggttaatccataatgtaaaattacattcggattaggctttccaaaagataaataaatgtgcaggaagtatgttcggaggtgcaggaatcaattgcctcataacaccaattacaaacccattttggaaacttctattcttaccaatagttgttcaaataaggcccaaatgttgagaatattgggtctttccactgaaaccgcacaaatttgtggatcagaaacgaaactgcccaaaatttaaaatacaaatggacatttttaaaattccatctttcaatgcccactctagtttgagtaatagatttcattttcgtgaaaagtgaaggatgctaatgcagttccacgaatggttctcagaaattcttaacgtcaagtttcttgcattgtgcggagatattaatgaaaactcaatgtctaaaatctgatgagatcttagcaaaaattctggagacggtcacttcaaaattgacagcaaataatgtagtaagctaccatattgaaagttgcaagtaatgcacccagcatgtgtttgatgccgtagaaagtggatagaatagcctcaagttgttgagagataagaaagccaaggtccaaaaatctgatcgaccaaccattttcatttagatgtcattatacatgctgacatggcatatatatatatatatatatatatatatatatatatatatatatatatatatatatatatatgcttttattgctttatactattataacaacttcaagttggatatacaacaaataacaactaacgaataattgattcaagttacattaaatttatttatttcacctaagattttaggttaaatctcgtgaatagaaaaaatatggatgtaaaagaagaatttcagtaaagatagtcagatctcttaacataaattaatcattgacaaatatttcatattaataacatgattataaaaaaaatataatgataattgaggtgaagagaatttcaagtcaagttggatggacaaatttgactatttcaaatttaatctaaaacctagctcaatttattaaatgagacatacaaattgtttgagaaaaaaaacggactaacgtactaatctatttcattacaagccggcctacgtggatcaagttaaatcaaaattaaattgagttcaaaattttcaaattcaaccaacacattttagcttattaaattaaacggatctacttattccaacaaccttagtttaagtaacctatagactatagttaagaaattgtcacactttttataatattttacattatttttggtgatatatttttccattattattattacacaaatattcaaaaaaaaatcgcaccctaaaatttacatctattttatatcattttctatttctctttattttttattatatcatctatcagattcacaatcattccctttcttttatttctctcttaaattccaaaatatatacaacatttagagtgtaaaataaatgtttttcatagaaattagcccataatagagtttgtacatggctagatactgaactaatgcatataatttcttaccttgccaacataatatgaaaagcagaaagtatttttttaaaagaaaaagtagaaagtattttgtacattaccaatacgtacttttaaatataaggtatcacaaatataaatataaatacttagaaaagattgatttaactttagagaagatttttatgataaaacataaaattacatacgtttaaaatttaaaattttaaaatataaaaaagcgtttaactaataaaaataatattttaatagtaataatttttatttaataatattttaatagtaatgctcaaaattttaaaatatctgcttattttaatatttcttgaattaagaaagagaaaatactaatttaacatttatcatcattcatcaagtaagaaagatacatgaatacatcaaaagataagtacttttacagatgatattatttaaatagcctaattttttttaaaaaaatgtatcttatctaatttcaatttaaaaaaaaaatgagctatattaattactaaagcatcttaggcccgccgtaaaattggccaaattattcattttaggccattattccaaaaactttgagcaaccactgactattataaagaatatatcatattccaactgatgtacgaatattcttaaaacctagcacgtttgtgaattttattccacctctatctttatttcttgtgctattttcttcttcttttgtttaaccaaaaagggtagccaaggaataagataccgatccgatgccatttcagaatcttgcttgttctcaactttcaactctcagagttaaaaataatattccactttgatataagaatatgactatgacatctattgtataatttagatccgagaatatgactatgacattacgccaattaatgtcttgaggagtagtatgtttgccttcatcccttaaaatagttcctatatatgctaatttacttccacaattcaacattgaaattggaagtctaagatatagaaatggaaatatttaagcatgtgaaaactaagcccaattggaattcattttgaatcagtcttaattaatatttcttccccacagttttgatttagacaactcatttttttaagcttgaaagttgaaattaagtagtacttatcctttgcagcgatgtaatgtattattactcgttaacaccatatttcagtttctaatatagataacacaaacgtagggttatatgtaaatttatgataaggacgcaacacaggcttaaaaattcatatatcagtggtggtatatgataatgtgatatgaatagactgcctcttacttatcttatctcataagctagagaaaaagaaaaagaaaaagaaaaaagtaaaggtcatggaaaaatgtagacagtgatgtggaaaccggagaaaaaacaaaggaaatgacttgacacaagaatggaactgactaagacgcgaaaatagtataacttccctcaattatatatatatatataaaaatacatatatttggcaattgggatgagtttgtgttatgttattaaacgtaaatatcaaataacttcaattttgagtcgtgtggttaactaatggcgtcattggtatagagccatcaaatttaagttgcttactgtcttcaaaattaacttgaatgcttagattaggttgtatctaaatgttccacactaagataaatcttagctttttttagcagctaaggctaatggattttaaccaaaatgattcataagtggggggtgtgtactgataatagagggggaagtggcatgttagtggtggattctgttattttctccttattatcttaatttgtcggtgtagctactaagcttcttcctctgctggttatccaatatttgtatttcatcatatacaaatatattatattgtgtaacttcgtttatgtattgctgattggtacttctggtacctcttatataaattgcctttttttctgattgaaaaaaacaaacaaacataagtgacgggaactggtgtttgatagatgataacaacacctaaaataaattaaagtcatggacaatttcggtgactatatagattcctccaaccactaactatgtatttggaaaccacttccatatcaagatgtggaaaacttccttattaatgcgtgaccaatcacacccttaactaataactaattcaattctaagataaaaacttgctgtagcttgatctacttgtattaatagttatagcaataacgataattttttattctacgaaccaactgcacaagttaaaataataaaaataaaaatattacattaatacagaagaccttctttttcatgttttgactctttgatttaagtacttaatgggtgtacaacaataataacagcaatgaactgaataaataagcaaagaaaaactgagtggctattgacttattaaagtttagggcacttctttttttacaacagaatattcggtgaagtggtccccattctaaattatggtacaaacttttgcgcaaatatctaaaataatattttttttataatagggtttaatttttgtgttacacacttaaccttttcctaattgaactttattctacttatgagattatgataatagtatacgttaaaaagcctacgttgataaaggcctttttcaggagtttataagttttttttattagtttataagtatttttaattaaaataagtttatttgatatatgaaattacttttttgataatttattttttataagctacttcaagtattacttgaagtaatttataaaaaataagttaacttataagttaatgattttttttttcaattttatttttattattttatttgaaattttattttatcattttagtcaatttaaaattctcttatttttttattcattgtataaaaaaatcatctatctaattttatattctatatatacacaataaacacaaagtcaatttatcaattgtcattattacattatttcttaaggttataaaatctttcaattatattaactttataattatttcttgaattattttgtttttcttttccctaaaattatgacataatttgtttctttatatatatttttattttatcaattcaattcataaaaacaaaaattcttctttttattaactaaacttcacataaaattaaatgttcttttatattatttgacatttattagttaacttatacgttaatcttattaaatacttttaattaaatcagttaatttctaatcttttagctcttaacttataaattataaatttccaattaacttataagttatttataaaaaaattgtaaaaccacttatctttcaaacgatcgctttcctagcttaaaatttctcagcacttcacaattagcatcagatcactaatcagtgattttacatattatcacatattgtggcataataaataattcatattgataagactattcaggtttgttagtggaatccaatgatgaaatattccttaactgtgcaattgggaagcctgggccaaaacaccacctaaatggaaaaatggagcacataataaaaaagaaatgaaatttggatcctgtgtattttatttttaataaaagaatttggattctgttatcagtgttatgtgaatacttgtaataatccaagttcagggaaactcctataaagagaaaatcaaaattgtgtttatgcgatattctccaaagatgccaatgttatcatcatcaaacgatcactataccccgacagtatgtcctgttattggatttattctcttccttagttgaaaacaattacagaaaacaaatcgatcgggcttgaaaaaatgggaggaaaagtagaatggcatattgaaaacgaagtaggtgacgtatttgtgcacttaattaacttatcgatatccccactcaacgtgataacttctctctcctctccactgaatcaaattgtcaatacacacgaaattagggtatacaaataataatgtgtttttggtacaccacacataaaaaaaaaattcatttcagttttgtatataagattattattcctcctccagtatttaaattttaacataattgtatattcaattcttgaattcaaaatttcatttgtttttcaataaatacttaagatcacatattaaatcaaaataattctatattagttaatgcacatggtcggttgtatagtgtatttgaattggtggtaaaaaaatttaaatcaatataaaagtcatttccaaaaaatcacaatgagttgaactcacatgttaccaaacgcaccaagtaaaactatttatatcaagttcttcataattttttattaaatatttatttaaatgatgatatgtgaatatttataataataaataacttttattaaatatataatgtgtaaaataaaaaacggtctaacttctcatgtgtgtcaaaagcaacaccaacgtcgcccaaacatcccaaatcttaatccatggaaaatgttctttcatctctttcaaatcgaccattagttaaaacaacctaaaattcgatcaaggttaattgcccaacctcgaactcattagtactagtgtaaactatttgagcaattagtttaatggttagagtcgtatttcatattgatgtacatagacaaaaccaaaatcttaattaaaactaaattaattatcaaagtaaatttaagataaatttgcaacggagaacgatataaaaaaagttatatataatttgtgaaatcaaggttagtctaacgataaagtaggatctaaactgacttttgagagaattttttttttttacttaatttaaagagagatatcttctttcatttctctggtgggaaagaaaatctcctcaaatgggagaaaagtcctcctatgaaggtagaagctctccattgagggagaagcccccttaaaggaggctttgttgtgctctaatactagggttgttaaggtgtcattgttgttagggttgttgaggaagtacacttataatttttttaattatgttattgggtcaaagtatttatcagttttgtcgagattaatgtgcattcaagaattccattgatc

SEQ ID NO: 34 - protein, 179 aa, Artificial Sequence
  SYNTHESIZED
MESVKFINMNAKHSSIFLLAMIFVLILASANAKIHEHEFVVEATPVKRLCKTHNSITVNGQYPGPTLEINNGDTLVVKVTNKARYNVTIHWHGVRQMRTGWADGPEFVTQCPIRPGGSYTYRFTVQGQEGTLWWHAHSSWLRATVYGALIIRPREGEPYPFPKPKHETPILLGNNLKKN

SEQ ID NO: 35 - protein, 849 aa, Artificial Sequence
  SYNTHESIZED
MLLVEKTNLTSQCFNRISDKKKERWKTHNNNPCRVLFLLCMWSLVVLPSCVRPALCEDESWDGVVVTASNLLALQAFKQELVDPEGFLRSWNDSGYGACSGGWVGIKCAQGQVIVIQLPWKGLKGRITDKIGQLQGLRKLSLHDNQIGGSIPSTLGLLPNLRGVQLFNNRLTGSIPSSLGFCPLLQSLDLSNNLLTGAIPYSLANSTKLYWLNLSFNSFSGTLPTSLTHSFSLTFLSLQNNNLSGNLPNSWGGSPKSGFFRLQNLILDHNFFTGNVPASLGSLRELSEISLSHNKFSGAIPNEIGTLSRLKTLDISNNAFNGSLPVTLSNLSSLTLLNAENNLLENQIPESLGTLRNLSVLILSRNQFSGHIPSSIANISMLRQLDLSLNNLSGEIPVSFESQRSLDFFNVSYNSLSGSVPPLLAKKFNSSSFVGNIQLCGYSPSTPCLSQAPSQGVIAPTPEVLSEQHHRRNLSTKDIILIVAGVLLVVLIILCCILLFCLIRKRSTSKAENGQATGRAATGRTEKGVPPVSAGDVEAGGEAGGKLVHFDGPLAFTADDLLCATAEIMGKSTYGTVYKAILEDGSQVAVKRLREKITKGHREFESEVSVLGKVRHPNVLALRAYYLGPKGEKLLVFDYMPKGGLASFLHGGGTETFIDWPTRMKIAQDMTRGLFCLHSLENIIHGNLTSSNVLLDENTNAKIADFGLSRLMSTAANSNVIATAGALGYRAPELSKLKKANTKTDIYSLGVILLELLTRKSPGVSMNGLDLPQWVASIVKEEWTNEVFDADMMRDASTVGDELLNTLKLALHCVDPSPSVRPEVHQVLQQLEEIRPERSVTASPGDDTI

SEQ ID NO: 36 - protein, 512 aa, Artificial Sequence
  SYNTHESIZED
MAYNFPDVFCWIQSLPPISEWETSSMSLNICSSSSSSCQPRLNLTVSKNNSNNHSSSNLYFVIIADCNIPIHLWTSKPFKPSSTTITNKTHNNNKLIDDEETISNLFVNFIQAILLYGSNKNSTPFLRFPNLDSITSNNFSDVFNLSFFTLLFLVCIYEAPAADFRSGCISNLKDHLTGFQSRQASHKIMKLLGSNLEEHWMRSLNLAVTNWVGELEAHNNPFRTPCPLFSYAFSTIGLWKVQLYCPLLVMDVENSKSNPASERLQFSLRYHHVEGVLQFNHKVLIKEEWAEIMVDIDNIRCDVIKLVNESLMSQRGVGAAEKHFPSRISLQLTPTLQDQVLSLSVGKSSENPRKEIGVDKSVEASFEPSNPLALKVSAGESSTVSLKPWKFEESVYGYSANLNWFLHDSVDGKEVFSSKPSKFAMLNPKSWFKNRYSSAYRPFNKEGGVIFAGDEYGEKVWWKVDKGAIGKTMEWEIRGWIWLTYWPNKRVTFYNETRRLEFREIVHLDVA

SEQ ID NO: 37 - protein, 226 aa, Artificial Sequence
  SYNTHESIZED
MGEIKEECTSLDKSEQEKQLHSKVSKVALYSSGESKAIASACETASSFGSVGPKVSHLKWGRWYKLRELEVATNGLCEENVIDEGGYRIVYHGLFPDGTKIAGDLDLICPKVFGAPGGSHYAHYHLVGRRTIILGSSSKARREILAEMGYEFTVMTADIDEKGIRREKPEDLVMALVEAKRCSALYIGLSVVIDVECLSFYIGLSTTVVENLAFYIDPAVVTVIEC

SEQ ID NO: 38 - protein, 305 aa, Artificial Sequence
  SYNTHESIZED
MAKKLARFGQFYHCNQMLTHDSWISDPIGTMSHVRASLEKQAVVPIHNAGWNSKSRLFIQHLAYGQKHINSHTKGKNTLISCGKTAEAINASKSDASSDNTPQGSLEKKPLQTATFPNGFEALVLEVCDETEIAELKVKVGDFEMHIKRNIGATKVPLSNISPTTPPPIPSKPMDESAPGSLPPSPPKSSPEKNNPFANVSKEKSPRLAALEASGTNTYVLVSSPTVGLFRRGRTVKGKKQPPICKEGDVIKEGQVIGYLDQFGTGLPIKSDVAGEVLKLLVEDGEPVGYGDPLIAVLPSFHDIK

SEQ ID NO: 39 - protein, 113 aa, Artificial Sequence
  SYNTHESIZED
MKIGKYKDLSSQLGPPPSIRVIEFKILMAGMPNKLATYPSGGLWLPVEVLSLFVTINDDDVVEGKFFLGLTVDAKCSYHIQQDLLTMVLFHKPLHELRWEGTCKDTLMLKLEA

SEQ ID NO: 40 - protein, 674 aa, Artificial Sequence
  SYNTHESIZED
MKPHTTASSFATSLPHVPCFRGTTAARATPSEPHHDSAGGLEFRRVSTSKRRLINLSVRHASRVTAASNPGGSDGDGDTRARSCRRGVLMTPFLVAGASILLSAATATARADEKAAESAPAPAAPEEPPKKKEEEEVITSRIYDATVIGEPLAIGKEKGKIWEKLMNARVVYLGEAEQVPVRDDRELELEIVKNLHRRCLEKEKRLSLALEVFPANLQEPLNQYMDKKIDGDTLKSYTLHWPPQRWQEYEPILSYCHENGIRLVACGTPLKILRTVQAEGIRGLTKDERKLYAPPAGSGFISGFTSISRRSSVDSTQNLSIPFGPSSYLSAQARVVDEYSMSQIILQNVLDGGVTGMLIVVTGASHVTYGSRGTGVPARISGKIQKKNQAVILLDPERQFIRREGEVPVADFLWYSAARPCSRNCFDRAEIARVMNAAGQRRDALPQDLQKGIDLGLVSPEVLQNFFDLEQYPLISELTHRFQGFRERLLADPKFLHRLAIEEAISITTTLLAQYEKRKENFFQEIDYVITDTVRGSVVDFFTVWLPAPTLSFLSYADEMKAPDNIGSLMGLLGSIPDNAFQKNPAGINWNLNHRIASVVFGGLKLASVGFISSIGAVASSNSLYAIRKVFNPAVVTEQRIMRSPILKTAVIYACFLGISANLRYQAVLEVDGG

SEQ ID NO: 41 - protein, 271 aa, Artificial Sequence
  SYNTHESIZED
MSTSSSSQSLKIGIVGFGNFGQFLAKTMIKQGHTLTATSRSDYSQLCLQMGIHFFRDVSAFLAADIDVIVLCTSILSLSEVVGSMPLTSLKRPTLFVDVLSVKEHPRELLLRELPEDSDILCTHPMFGPQTANNGWADHTFMYDKVRIRDEATCSSFIQIFATEGCKMVQMSCEEHDRAAAKSQFITHTIGRTLGEMDIQSTPIDTKGFETLVKLKETMMRNSFDLYSGLFVYNRFARQELENLEHAFYKVKETLMIQRSNGEQGHKRTES

SEQ ID NO: 42 - protein, 776 aa, Artificial Sequence
  SYNTHESIZED
MAAASEIGNKSMVSLMTMVTQFMDACLKSLGQSSIVSQIMGGVLFGPSMLGNKKILGLALFPMKGAVVLDTVSLFGLMFFFFIWCVKMDIATLMKTEKVAITLGISVFAFTLIIPTGLAFLMMKYIAMDGSLAKALPFLAMSQTLTVFISIAVLLTDLKVLNTDIGRLTMSAAMFADVAGFILTVILFAILQDQSGSFVRLACILLSIVGVWLLVIFVMRPTIIWMVKHPGRGSVNEICLVCIFLLVLLSAFVSELIGQHFIMGPILLGLAVPEGPPIGTALMSKLETICTAFLYPIFLAVNGLQTDFFKINKQSLWIVCVILIVAFFVKIGAVMLPGYYYNLPLKQCFVIGLFLNGRGIAELATYNMWKRGKLISEQEFALMVASIIVVNCILVPLIRYIYDPSELYQTGRRCTIQHTRRDLELRVMVCIHNNENLPMILNLLEASYASRESRIEVTALVLVELQGRARPILFANQEQPHDEMRSMSCNASHIDNALRQYAQQNEGYVSVQSFTSISTFETMYDDICKISLDTGSNILILPFHKRWEIDATVEISHRTIQTMNIEVLERAPCSVGILVDRGILSPSPSLLMARAAFYVAVFFIGGQDDAETLAYASRMVRHECVYVTVVRFLLFGHENSKDRKRDSDLIDEYRYYNAGNQRFELMNEVVKNGIEMSTCIRRLIDYFDLVMVGREHPDSVIFQGHDQWSECQELGVIGDMLASPDFVTKASLLVVQQQRIRGTLVKHNVNANPVPNHRDQLVYDIPNDTVDKYDRV

SEQ ID NO: 43 - protein, 74 aa, Artificial Sequence
  SYNTHESIZED
MANVVEVKVGLHCEDCIKKILKAIKKIEDIETYNVDTQLNKVMVTGNVTTEQVIRVLQKIGKNATAWEDAQTNK

SEQ ID NO: 44 - protein, 152 aa, Artificial Sequence
  SYNTHESIZED
MGKYLESAARLEELSRIVSSAAKPNRSKGTLPRSPNRVATPRIANPIGVKVEPAKKMEPLECRTPLAKVVADCSKRWFQDTLKEAKAGDSAMQVLVGQMYYSGYGVPRDPHKVTVYTVTNFVYRGMFGLVKHQEIEIQFGKCAVNGQVKGAL

SEQ ID NO: 45 - protein, 35 aa, Artificial Sequence
  SYNTHESIZED
MRVPFTTHNSRKNISHAVKYLSTESGRWGEMAESM

SEQ ID NO: 46 - DNA, 22 bp, Artificial Sequence
  SYNTHESIZED
cagtgaaacttggagatgccag

SEQ ID NO: 47 - DNA, 23 bp, Artificial Sequence
  SYNTHESIZED
tcagtctcaaattagtgagggat

SEQ ID NO: 48 - DNA, 31 bp, Artificial Sequence
  SYNTHESIZED
aagtgtgatacacactatccctcctcctcgt

SEQ ID NO: 49 - DNA, 33 bp, Artificial Sequence
  SYNTHESIZED
aagtgtgatacatactatccctcctcctcgtgc

SEQ ID NO: 50 - DNA, 22 bp, Artificial Sequence
  SYNTHESIZED
ggttgtgacagcatcaaacctc

SEQ ID NO: 51 - DNA, 20 bp, Artificial Sequence
  SYNTHESIZED
ccttccaaggaagctggatc

SEQ ID NO: 52 - DNA, 32 bp, Artificial Sequence
  SYNTHESIZED
taacctgtccctgagcacacttgattccaacc

SEQ ID NO: 53 - DNA, 37 bp, Artificial Sequence
  SYNTHESIZED
cacaataacctgtcccttagcacacttgattccaacc

SEQ ID NO: 54 - DNA, 36 bp, Artificial Sequence
  SYNTHESIZED
gctttcaagcaagagttggctgatccagaagggttc

SEQ ID NO: 55 - DNA, 38 bp, Artificial Sequence
  SYNTHESIZED
agctttcaagcaagagttggttgatccagaagggttct

SEQ ID NO: 56 - DNA, 21 bp, Artificial Sequence
  SYNTHESIZED
ccatcataggaagctaagcac

SEQ ID NO: 57 - DNA, 20 bp, Artificial Sequence
  SYNTHESIZED
gcacacaagagatcatcagc

SEQ ID NO: 58 - DNA, 40 bp, Artificial Sequence
  SYNTHESIZED
ggagttctcctcgtagtcctgattatactttgttgtgtcc

SEQ ID NO: 59 - DNA, 40 bp, Artificial Sequence
  SYNTHESIZED
ggagttctcctcgtagttctgattatactttgttgtgtcc

SEQ ID NO: 60 - DNA, 24 bp, Artificial Sequence
  SYNTHESIZED
cagcatcaaacctcttagcacttg

SEQ ID NO: 61 - DNA, 20 bp, Artificial Sequence
  SYNTHESIZED
cattccagctccgcaagaac

SEQ ID NO: 62 - DNA, 16 bp, Artificial Sequence
  SYNTHESIZED
ctggatcagccaactc

SEQ ID NO: 63 - DNA, 17 bp, Artificial Sequence
  SYNTHESIZED
tctggatcaaccaactc

SEQ ID NO: 64 - DNA, 25 bp, Artificial Sequence
  SYNTHESIZED
ccagttttgtgtgaagatgaaggtt

SEQ ID NO: 65 - DNA, 25 bp, Artificial Sequence
  SYNTHESIZED
gcttcaagtgctaagaggtttgatg

SEQ ID NO: 66 - DNA, 16 bp, Artificial Sequence
  SYNTHESIZED
ctgtcacaaccactcc

SEQ ID NO: 67 - DNA, 16 bp, Artificial Sequence
  SYNTHESIZED
ctgtcacagccactcc

SEQ ID NO: 68 - DNA, 21 bp, Artificial Sequence
  SYNTHESIZED
cggaggttgggttggaatcaa

SEQ ID NO: 69 - DNA, 21 bp, Artificial Sequence
  SYNTHESIZED
ccctcaaacccttccaaggaa

SEQ ID NO: 70 - DNA, 16 bp, Artificial Sequence
  SYNTHESIZED
ctgtccctgagcacac

SEQ ID NO: 71 - DNA, 17 bp, Artificial Sequence
  SYNTHESIZED
cctgtcccttagcacac

SEQ ID NO: 72 - DNA, 23 bp, Artificial Sequence
  SYNTHESIZED
tgggaattccaagaatggcttct

SEQ ID NO: 73 - DNA, 21 bp, Artificial Sequence
  SYNTHESIZED
gcaggaacgtcaccagtgaaa

SEQ ID NO: 74 - DNA, 20 bp, Artificial Sequence
  SYNTHESIZED
ttgatcctagatcataactt

SEQ ID NO: 75 - DNA, 20 bp, Artificial Sequence
  SYNTHESIZED
ttgatcctagatcataactt

SEQ ID NO: 76 - DNA, 22 bp, Artificial Sequence
  SYNTHESIZED
cggccactatgaggacagaaaa

SEQ ID NO: 77 - DNA, 19 bp, Artificial Sequence
  SYNTHESIZED
cctccccacctgcttcaac

SEQ ID NO: 78 - DNA, 17 bp, Artificial Sequence
  SYNTHESIZED
cagttgctggtggtgat

SEQ ID NO: 79 - DNA, 17 bp, Artificial Sequence
  SYNTHESIZED
cagttgctgttggtgat

SEQ ID NO: 80 - DNA, 30 bp, Artificial Sequence
  SYNTHESIZED
ctacagtcttggtgttatcttgttagaact

SEQ ID NO: 81 - DNA, 22 bp, Artificial Sequence
  SYNTHESIZED
gcaacccactgaggcaaatcta

SEQ ID NO: 82 - DNA, 17 bp, Artificial Sequence
  SYNTHESIZED
ccattcatagacacccc

SEQ ID NO: 83 - DNA, 17 bp, Artificial Sequence
  SYNTHESIZED
ccattcataggcacccc

SEQ ID NO: 84 - DNA, 27 bp, Artificial Sequence
  SYNTHESIZED
tgtccctaaataaattaataaatccaa

SEQ ID NO: 85 - DNA, 20 bp, Artificial Sequence
  SYNTHESIZED
aaatgaccctctctctctct

SEQ ID NO: 86 - DNA, 20 bp, Artificial Sequence
  SYNTHESIZED
agacaaagcattctgatcgc

SEQ ID NO: 87 - DNA, 20 bp, Artificial Sequence
  SYNTHESIZED
agattcgccactactgttgg

SEQ ID NO: 88 - DNA, 21 bp, Artificial Sequence
  SYNTHESIZED
cccaacataattccaacttca

SEQ ID NO: 89 - DNA, 20 bp, Artificial Sequence
  SYNTHESIZED
gcaacaatgctagccatcaa

SEQ ID NO: 90 - DNA, 23 bp, Artificial Sequence
  SYNTHESIZED
ttccaacttcaaaattcactcaa

SEQ ID NO: 91 - DNA, 20 bp, Artificial Sequence
  SYNTHESIZED
gtgtcatcaacaacgcaaca

SEQ ID NO: 92 - DNA, 20 bp, Artificial Sequence
  SYNTHESIZED
gcactaatggtgacacacac

SEQ ID NO: 93 - DNA, 22 bp, Artificial Sequence
  SYNTHESIZED
tgagttagacctcctctcttgt

SEQ ID NO: 94 - DNA, 24 bp, Artificial Sequence
  SYNTHESIZED
ctttaaagtctcaagtcattggat

SEQ ID NO: 95 - DNA, 20 bp, Artificial Sequence
  SYNTHESIZED
cgaaagggcctctttatgtt

SEQ ID NO: 96 - DNA, 21 bp, Artificial Sequence
  SYNTHESIZED
ccttgcaatagaggagtacaa

SEQ ID NO: 97 - DNA, 25 bp, Artificial Sequence
  SYNTHESIZED
agtacgtgtcttgattttatttctt

SEQ ID NO: 98 - DNA, 20 bp, Artificial Sequence
  SYNTHESIZED
gttttggcaagcaagagtcc

SEQ ID NO: 99 - DNA, 25 bp, Artificial Sequence
  SYNTHESIZED
aactagtatggtataaagtaaggct

SEQ ID NO: 100 - DNA, 21 bp, Artificial Sequence
  SYNTHESIZED
gcatgtttggctcgcattaaa

SEQ ID NO: 101 - DNA, 22 bp, Artificial Sequence
  SYNTHESIZED
tcccacttcaattaactcatgc

SEQ ID NO: 102 - DNA, 20 bp, Artificial Sequence
  SYNTHESIZED
cacctgcatcaagatgaaca

SEQ ID NO: 103 - DNA, 20 bp, Artificial Sequence
  SYNTHESIZED
gcctattacttgggacccaa

SEQ ID NO: 104 - DNA, 41 bp, Artificial Sequence
  SYNTHESIZED
ccaattccatggcgatcgaaggtcgtcttcaaggcctcagg

SEQ ID NO: 105 - DNA, 42 bp, Artificial Sequence
  SYNTHESIZED
cggtttctcgagctattagagaattatgtctttggtgcttag

SEQ ID NO: 106 - DNA, 36 bp, Artificial Sequence
  SYNTHESIZED
caccatcatcatcatcatcttcaaggcctcaggaag

SEQ ID NO: 107 - DNA, 36 bp, Artificial Sequence
  SYNTHESIZED
cttcctgaggccttgaagatgatgatgatgatggtg

SEQ ID NO: 108 - protein, 28 aa, Artificial Sequence
  SYNTHESIZED
CTLSRLKTLDISNNALNGNLPATLSNLS